(19) United States
(12) Patent Application Publication
(10) Pub. No.: US 2006/0234344 A1
(43) Pub. Date: Oct. 19, 2006

(54) Protein kinases

(75) Inventors: Gregory D. Plowman, San Carlos, CA (US); Ricardo Martinez, Foster City, CA (US); David Whyte, Belmont, CA (US); Sucha Sudarsanam, Greenbrae, CA (US); Gerard Manning, La Jolla, CA (US)

Correspondence Address: FOLEY AND LARDNER LLP;SUITE 500, 3000 K STREET NW, WASHINGTON, DC, 20007, US

(73) Assignee: SUGEN, INC.

(21) Appl. No.: 11/377,316

(22) Filed: Mar. 17, 2006

Related U.S. Application Data
(62) Division of application No. 09/979,167, filed on Jul. 26, 2002.
(60) Provisional application No. 60/136,503, filed on May 28, 1999.

(51) Int. Cl. C07H 21/04; C12P 21/06; C12N 9/12

(52) U.S. Cl. 435/69.1; 435/194; 435/320.1; 435/325; 536/23.2

(57) ABSTRACT

The present invention relates to novel kinase polypetides, nucleotide sequences encoding the novel kinase polypetides, as well as various products and methods useful for the diagnosis and treatment of various kinase-related disease and conditions.

### FIELD OF THE INVENTION

The present invention relates to novel kinase polypeptides, nucleotide sequences encoding the novel kinase polypeptides, as well as various products and methods useful for the diagnosis and treatment of various kinase-related diseases and conditions.

### BACKGROUND OF THE INVENTION

The following description of the background of the invention is provided to aid in understanding the invention, but is not admitted to be or to describe prior art to the invention.

Cellular signal transduction is a fundamental mechanism whereby external stimuli that regulate diverse cellular processes are relayed to the interior of cells. One of the key biochemical mechanisms of signal transduction involves the reversible phosphorylation of proteins, which enables regulation of the activity of mature proteins by altering their structure and function.

Protein phosphorylation plays a pivotal role in biological signal transduction. Among the biological functions controlled by protein phosphorylation are the following: cell division; differentiation and death (apoptosis); cell motility and cytoskeletal structure; control of DNA replication, transcription, splicing and translation; protein translocation events from the endoplasmic reticulum and Golgi apparatus to the membrane and extracellular space; protein nuclear import and export; regulation of metabolic reactions, etc. Abnormal protein phosphorylation is widely recognized to be causally linked to the etiology of many diseases including cancer as well as immunologic, neuronal and metabolic disorders.

The most common phospho-acceptor amino acid residues are serine, threonine and tyrosine. Phosphorylation in histidine has also been observed in bacteria. The presence of a phosphate moeity modulates protein function in multiple ways. A common mechanism includes changes in the catalytic properties (Vₘₐₓ and Kₘ) of an enzyme leading to its activation or inactivation. A second widely recognized mechanism involves promoting protein-protein interactions. An example of this is the tyrosine autophosphorylation of the ligand-activated EGF receptor tyrosine kinase. This event triggers the high-affinity binding to the phosphotyrosine residue on the receptor's C-terminal intracellular domain to the SH2 motif of the adaptor molecule Grb2. Grb2 in turn binds through its SH3 motif to a second adaptor molecule, such as SHC. The formation of this ternary complex acivates the signaling events that are responsible for the biological effects of EGF. Serine and threonine phosphorylation events have also being recently recognized to exert their biological function through protein-protein interaction events mediated by the high-affinity binding of phosphoserine and phosphothreonine to WW motifs present in a large variety of proteins (Lu, P. J. et al. (1999) Science 283:1325-1328). A third important outcome of protein phosphorylation is changes in the subcellular localization of the substrate. As an example, nuclear import and export events in a large diversity of proteins are regulated by protein phosphorylation (Drier E. A. et al. (1999) Genes Dev 13: 556-568).

Protein kinases are one of the largest families of eukaryotic proteins with several hundred known members. These proteins share a 250-300 amino acid domain that can be subdivided into 12 distinct subdomains that comprise the common catalytic core structure. These conserved protein motifs have recently been exploited using PCR-based and bioinformatic strategies leading to a significant expansion of the known kinases. Multiple alignment of the sequences in the catalytic domain of protein kinases and subsequent parsimony analysis permits their segregation into a dendrogram reflecting the relatedness of their catalytic domains (FIG. 1). In this manner, related kinases are clustered into distinct branches or subfamilies including: tyrosine kinases, cyclic-nucleotide-dependent kinases, calcium/calmodulin kinases, cyclin-dependent kinases and MAP-kinases, serine-threonine kinase receptors, and several other less defined subfamilies.

We have recently completed a systematic analysis of the protein kinases present in C. elegans, the multicellular organism whose entire DNA sequence has been determined. We identified 473 unique kinase profiles including 398 full-length conventional kinases, and 20 additional proteins that may function as atypical protein kinases. (Plowman G. D. et al. (1999), Proc. Natl. Acad. Sci. 96:13603-13610).

Using parsimony analysis, the protein kinases may be divided into 4 major groups: AGC, CAMK, CMGC and tyrosine kinases. In addition, there are a number of minor yet distinct families, including the STE and casein kinase 1, families related to worm- or fungal-specific kinases, and a family designated “other” to represent several smaller families. In addition, we designate an “atypical” family to represent protein kinases whose catalytic domain has little or no primary sequence homology to conventional kinases, including the A6 kinases and PI3 kinases.

The AGC kinases are basic amino acid-directed enzymes that phosphorylate residues found proximal to Arg and Lys. Examples of this group are the cyclic nucleotide-dependent kinases, G protein kinases, NDR or DBF2 and the ribosomal S6 kinases.

The CAMK group kinases are also basic amino acid-directed kinases. They include the Ca2+/calmodulin-regulated and AMP-dependent protein kinases, myosin light chain kinases, checkpoint 2 kinases (CHK2) and EMK-related protein kinases. The EMK family of STK are involved in the control of cell polarity, micotubule stability and cancer. One member of the EMK family, C-TAK1 has been reported to control entry into mitosis by activating Cdc25C which in turn dephosphorylates Cdc2.

CMGC group kinases are “proline-directed” enzymes phosphorylating residues that exist in a proline-rich context. They include the cyclin-dependent kinases (CDKs), mitogen-activated kinases (MAPKs), GSK3s and CLKs. Most CMGC kinases have larger-than-average kinase domains owing to the presence of insertions within subdomains X and XI.

The tyrosine kinase group encompass both cytoplasmic (i.e. src) as well as transmembrane receptor tyrosine kinases (i.e. EGF receptor). These kinases play a pivotal role in the signal transduction processes that mediate cell proliferation, differentiation and apoptotis.

Group members that define smaller, yet distinct phylogenetic branches of conventional kinases include the elongation factor 2 kinases (EIFKs); homologues of the yeast sterile family kinases (STE) which refers to 3 classes of kinases which lie sequentially upstream of the MAPKs; mixed lineage kinases (MLKs); Lim-domain containing kinases (LIMKs); Calcium-calmodulin kinase kinases (CAMKK), dual-specific tyrosine kinases (DYRK), integrin receptor associated kinase (IRAK); testis-specific kinases (TSK); UNC-51 related kinases (UNC); several families that are close homologues to worm (C26C2.1, YQ09, ZC581.9, YFL033c, C24A 1.3), Drosophila (SLOB), or yeast (YDOD_sp, YGR262_sc) kinases, and others that are “unique” and don't cluster into any obvious family.

### SUMMARY OF THE INVENTION

Through a search of the EST database for homologies to the conserved catalytic kinase domain of protein kinases, hundreds of mammalian members of known and previously unidentified protein kinase families and groups have been identified as part of the present invention. Multiple alignment and parsimony analysis of the catalytic domain reveals that approximately half of these protein kinases cluster into 10 known groups, with the other half perhaps defining novel groups. Classification in this manner has proven highly accurate not only in predicting motifs present in the remaining non-catalytic portion of each protein, but also in their regulation, substrates, and signaling pathways. The present invention includes the partial or complete sequence of new protein kinases, their classification, predicted or deduced protein structure, and a strategy for elucidating their biologic and therapeutic relevance.

Thus, a first aspect of the invention features an isolated, enriched, or purified nucleic acid molecule encoding a kinase polypeptide selected from the group consisting SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242.

By “isolated” in reference to nucleic acid is meant a polymer of nucleotides conjugated to each other, including DNA and RNA, that is isolated from a natural source or that is synthesized. The isolated nucleic acid of the present invention is unique in the sense that it is not found in a pure or separated state in nature. Use of the term “isolated” indicates that a naturally occurring sequence has been removed from its normal cellular (ie., chromosomal) environment. Thus, the sequence may be in a cell-free solution or placed in a different cellular environment. The term does not imply that the sequence is the only nucleotide chain present, but that it is essentially free (about 90-95% pure at least) of non-nucleotide material naturally associated with it, and thus is distinguished from isolated chromosomes.

By the use of the term “enriched” in reference to nucleic acid is meant that the specific DNA or RNA sequence constitutes a significantly higher fraction (2-5 fold) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA sequences present, just that the relative amount of the sequence of interest has been significantly increased. The term “significant” is used to indicate that the level of increase is useful to the person making such an increase, and generally means an increase relative to other nucleic acids of about at least 2 fold, more preferably at least 5 to 10 fold or even more. The term also does not imply that there is no DNA or RNA from other sources. The other source DNA may, for example, comprise DNA from a yeast or bacterial genome, or a cloning vector such as pUC19. This term distinguishes from naturally occurring events, such as viral infection, or tumor type growths, in which the level of one mRNA may be naturally increased relative to other species of mRNA. That is, the term is meant to cover only those situations in which a person has intervened to elevate the proportion of the desired nucleic acid.

It is also advantageous for some purposes that a nucleotide sequence be in purified form The term “purified” in reference to nucleic acid does not require absolute purity (such as a homogeneous preparation). Instead, it represents an indication that the sequence is relatively more pure than in the natural environment (compared to the natural level this level should be at least 2-5 fold greater, e.g., in terms of mg/mL). Individual clones isolated from a cDNA library may be purified to electrophoretic homogeneity. The claimed DNA molecules obtained from these clones could be obtained directly from total DNA or from total RNA. The cDNA clones are not naturally occurring, but rather are preferably obtained via manipulation of a partially purified naturally occurring substance (messenger RNA). The construction of a cDNA library from mRNA involves the creation of a synthetic substance (cDNA) and pure individual cDNA clones can be isolated from the synthetic library by clonal selection of the cells carrying the cDNA library. Thus, the process which includes the construction of a cDNA library from mRNA and isolation of distinct cDNA clones yields an approximately 10⁶-fold purification of the native message. Thus, purification of at least one order of magnitude, preferably two or three orders, and more preferably four or five orders of magnitude is expressly contemplated.

By a “kinase polypeptide” is meant 10 (preferably 20, more preferably 40, most preferably 75) or more contiguous amino acids set forth in an amino acid sequence selected from the group consisting of those set forth in SEQ ID NO:122,SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or functional derivatives thereof as described herein. For sequences for which the full-length sequence is not given, the remaining sequences can be determined using methods well-known to those in the art and are intended to be included in the invention. In certain aspects, polypeptides of 100, 200, 300 or more amino acids are preferred. The kinase polypeptide can be encoded by a full-length nucleic acid sequence or any portion of the full-length nucleic acid sequence, so long as a functional activity of the polypeptide is retained. By “functional” domain is meant any region of the polypeptide that may play a regulatory or catalytic role as predicted from amino acid sequence homology to other proteins or by the presence of amino acid sequences that may give rise to specific structural conformations (i.e., coiled-coils). For some purposes, polypeptide domains are preferred, including, but not limited to, N-terminal, catalytic/kinase and C-terminal.

The amino acid sequence will be substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to a sequence selected from the group consisting of those set forth in SEQ ID NO:122, SEQ ID NO:123, SEQ ID NO:124, SEQ ID NO: 125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 or portions of or the entire corresponding full-length amino acid sequences.

By “identity” is meant a property of sequences that measures their similarity or relationship. Identity is measured by dividing the number of identical residues between two sequences (either full-length or a defined domain) by the total number of residues in the known sequence, or the domain of the known sequence, and multiplying the product by 100. Thus, two copies of exactly the same sequence have 100% identity, but sequences that are less highly conserved, and have replacements and substitutions, have a lower degree of identity. “Gaps” are spaces in an alignment that can result from aligning a novel sequence with a known sequence when the novel sequence has additions or deletions of amino acids in comparison with the known sequence. These gaps do not factor into the assessment of % identity using the sbove calculation.

Those skilled in the art will recognize that several computer programs are also available for determining sequence identity using standard parameters, for example, Blast (Altschul, et al. (1997) Nucleic Acids Res. 25:3389-3402), Blast2 (Altschul, et al. (1990) J. Mol. Biol. 215:403-410), and Smith-Waterman (Smith, et al. (1981) J. Mol. Biol. 147:195-197).

In preferred embodiments, the invention features isolated, enriched, or purified nucleic acid molecules encoding a kinase polypeptide comprising a nucleotide sequence that: (a) encodes a polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to the sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; () is the complement of the nucleotide sequence of (a); (c) hybridizes under highly stringent conditions to the nucleotide molecule of (a) and encodes a naturally occurring kinase polypeptide; (d) encodes a kinase polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof. A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ D) NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to the sequence of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, except that it lacks one or more, but not all, of a domain selected from the group consisting of an N-terminal domain, a catalytic domain, a C-terminal domain, a coiled-coil structure region, a proline-rich region, a spacer region, an insert, and a C-terminal tail; (e) is the complement of the nucleotide sequence of (d); (f) encodes a polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof (The domain demarcations of the polypeptides of the invention are indicated in Table 2 by reference to the kinase domain.) A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to the sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; (b) is the complement of the nucleotide sequence of (a); (c) hybridizes under highly stringent conditions to the nucleotide molecule of (a) and encodes a naturally occurring kinase polypeptide; (d) encodes a kinase polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof. A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to a domain of a polypeptide selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ D) NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, where the domain is selected from the group consisting of an N-terminal domain, a catalytic domain, a C-terminal domain, a coiled-coil structure region, a proline-rich region, a spacer region, an insert, and a C-terminal tail; (g) is the complement of the nucleotide sequence of (f); (h) encodes a polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to the sequence selected from-the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; (b) is the complement of the nucleotide sequence of (a); (c) hybridizes under highly stringent conditions to the nucleotide molecule of (a) and encodes a naturally occurring kinase polypeptide; (d) encodes a kinase polypeptide having an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or fragments thereof A sequence that is substantially similar to a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 will have at least 75% identity (preferably 90%, more preferably at least 95% and most preferably 99-100%) to the sequence of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, except that it lacks one or more of the domains selected from the group consisting of a N-terminal domain, a catalytic domain, a C-terminal domain, a coiled-coil structure region, a proline-rich region, a spacer region, an insert, and a C-terminal tail; or (i) is the complement of the nucleotide sequence of (h). The domain demarcations of the polypeptides of the invention are indicated in Table 2 by reference to the kinase domain.

The term “complement” refers to two nucleotides that can form multiple favorable interactions with one another. For example, adenine is complementary to thymine as they can form two hydrogen bonds. Similarly, guanine and cytosine are complementary since they can form three hydrogen bonds. A nucleotide sequence is the complement of another nucleotide sequence if all of the nucleotides of the first sequence are complementary to all of the nucleotides of the second sequence.

The term “domain” refers to a region of a polypeptide that contains a particular function. For instance, N-terminal or C-terminal domains of signal transduction proteins can serve functions including, but not limited to, binding molecules that localize the signal transduction molecule to different regions of the cell or binding other signaling molecules directly responsible for propagating a particular cellular signal. Some domains can be expressed separately from the rest of the protein and function by themselves, while others must remain part of the intact protein to retain function. The latter are termed functional regions of proteins and also relate to domains.

The term “N-terminal domain” refers to the extracatalytic region located between the initiator methionine and the catalytic domain of the protein kinase. The N-terminal domain can be identified following a Smith-Waterman alignment of the protein sequence against the non-redundant protein database to define the N-terminal boundary of the catalytic domain. Depending on its length, the N-terminal domain may or may not play a regulatory role in kinase function. An example of a protein kinase whose N-terminal domain has been shown to play a regulatory role is PAK65, which contains a CRIB motif used for Cdc42 and rac binding (Burbelo, P. D. et al. (1995) J. Biol. Chem. 270, 29071-29074). The N-terminal domain of a protein kinase of the invention is that portion of the protein kinase to the amino-terminal side of the kinase domain where the kinase domain is identified in Table 2, herein. Further, in some cases, portions of the N-terminal domains of the protein kinases of the invention have not been identified since the entire sequence is not available. However, with the methods described herein, the full-length sequences of the kinases of the invention can be determined and using the approaches described herein the N-terminal domain can be identified.

The term “catalytic domain” or “kinase domain” refers to a region of the protein kinase that is typically 25-300 amino acids long and is responsible for carrying out the phosphate transfer reaction from a high-energy phosphate donor molecule such as ATP or GTP to itself (autophosphorylation) or to other proteins (exogenous phosphorylation). The catalytic domain of protein kinases is made up of 12 subdomains that contain highly conserved amino acid residues, and are responsible for proper polypeptide folding and for catalysis. The catalytic domain can be identified following a Smith-Waterman alignment of the protein sequence against the non-redundant protein database. The catalytic/kinase domains of the protein kinases of the invention are identified in Table 2, herein. Further, in some cases, the complete sequence of the catalytic/kinase domains of the protein kinases of the invention may not have been provided since the entire sequence is not available. However, with the methods described herein, the full-length sequences of the kinases of the invention can be determined, and using the approaches described herein, the catalytic/kinase domain can be identified.

The term “catalytic activity”, as used herein, defines the rate at which a kinase catalytic domain phosphorylates a substrate. Catalytic activity can be measured, for example, by determining the amount of a substrate converted to a phosphorylated product as a function of time. Catalytic activity can be measured by methods of the invention by holding time constant and determining the concentration of a phosphorylated substrate after a fixed period of time. Phosphorylation of a substrate occurs at the active-site of a protein kinase. The active-site is normally a cavity in which the substrate binds to the protein kinase and is phosphorylated.

The term “substrate” as used herein refers to a molecule phosphorylated by a kinase of the invention. Kinases phosphorylate substrates on serine/threonine or tyrosine amino acids. The molecule may be another protein or a polypeptide.

The term “C-terminal domain” refers to the region located between the catalytic domain and the carboxy-terminal amino acid residue of the protein kinase. The C-terminal domain can be identified by using a Smith-Waterman alignment of the protein sequence against the non-redundant protein database to define the C-terminal boundary of the catalytic domain or of any functional C-terminal extracatalytic domain. Depending on its length and amino acid composition, the C-terminal domain may or may not play a regulatory role in kinase function. An example of a protein kinase whose C-terminal domain may play a regulatory role is PAK3 which contains a heterotrimeric G_{b} subunit-binding site near its C-terminus (Leeuw, T. el al. (1998) Nature, 391, 191-195). The C-terminal domain of a protein kinase of the invention is that portion of the protein kinase to the carboxy-terminal side of the kinase domain where the kinase domain is identified in Table 2, herein. In some cases, the C-terminal domains of the protein kinases of the invention have not been provided since the entire sequence is not available. However, with the methods described herein, the full-length sequences of the kinases of the invention can be determined, and using the approaches described herein, the C-terminal domain can be identified.

The term “signal transduction pathway” refers to the molecules that propagate an extracellular signal through the cell membrane to become an intracellular signal. This signal can then stimulate a cellular response. The polypeptide molecules involved in signal transduction processes are typically receptor and non-receptor protein tyrosine kinases, receptor and non-receptor protein phosphatases, SRC homology 2 and 3 domains, phosphotyrosine binding proteins (SRC homology 2 (SH2) and phosphotyrosine binding (PTB and PH) domain containing proteins), proline-rich binding proteins (SH3 domain containing proteins), nucleotide exchange factors, and transcription factors.

The term “coiled-coil structure region” as used herein, refers to a polypeptide sequence that has a high probability of adopting a coiled-coil structure as predicted by computer algorithms such as COILS (Lupas, A. (1996) Meth. Enzymology 266:513-525). Coiled-coils are formed by two or three amphipathic α-helices in parallel. Coiled-coils can bind to coiled-coil domains of other polypeptides resulting in homo- or heterodimers (Lupas, A. (1991) Science 252:1162-1164). Coiled-coil-dependent oligomerization has been shown to be necessary for protein function including catalytic activity of serine/threonine kinases (Roe, J. et al. (1997) J. Biol. Chem. 272:5838-5845). Coiled-coil regions in the proteins of the invention can be identified using these methods. They may be present as sub-domains of the N-terminal, kinase, or C-terminal domains of the polypeptides of the invention.

The term “proline-rich region” as used herein, refers to a region of a protein kinase whose proline content over a given amino acid length is higher than the average content of this amino acid found in proteins (i.e., >10%). Proline-rich regions are easily discernable by visual inspection of amino acid sequences and quantitated by standard computer sequence analysis programs such as the DNAStar program EditSeq. Proline-rich regions have been demonstrated to participate in regulatory protein -protein interactions. Among these interactions, those that are most relevant to this invention involve the “PxxP” proline rich motif found in certain protein kinases (i.e., human PAK1) and the SH3 domain of the adaptor molecule Nck (Galisteo, M. L. et al. (1996) J. Biol. Chem. 271:20997-21000). Other regulatory interactions involving “PxxP” proline-rich motifs include the WW domain (Sudol, M. (1996) Prog. Biophys. Mol. Bio. 65:113-132). Proline rich regions in the proteins of the invention can be identified using these methods. They may be present as sub-domains of the N-terminal, kinase, or C-terminal domains of the polypeptides of the invention.

The term “spacer region” as used herein, refers to a region of the protein kinase located between predicted functional domains. The spacer region has no detectable homology to any amino acid sequence in the database, and can be identified by using a Smith-Waterman alignment of the protein sequence against the non-redundant protein database to define the C— and N-terminal boundaries of the flanking functional domains. Spacer regions may or may not play a fundamental role in protein kinase function. Precedence for the regulatory role of spacer regions in kinase function is provided by the role of the src kinase spacer in inter-domain interactions (Xu, W. et al. (1997) Nature 385:595-602). Spacer regions in the proteins of the invention can be identified using these methods. They may be present as sub-domains of the N-terminal, kinase, or C-terminal domains of the polypeptides of the invention.

The term “insert” as used herein refers to a portion of a protein kinase that is absent from a close homolog. Inserts may or may not by the product alternative splicing of exons. Inserts can be identified by using a Smith-Waterman sequence alignment of the protein sequence against the non-redundant protein database, or by means of a multiple sequence alignment of homologous sequences using the DNAStar program Megalign. Inserts may play a functional role by presenting a new interface for protein-protein interactions, or by interfering with such interactions. Insert regions in the proteins of the invention can be identified using these methods. They may be present as sub-domains of the N-terminal, kinase, or C-terminal domains of the polypeptides of the invention.

The term “C-terminal tail” as used herein, refers to a C-terminal domain of a protein kinase, that by homology extends or protrudes past the C-terminal amino acid of its closest homolog. C-terminal tails can be identified by using a Smith-Waterman sequence alignment of the protein sequence against the non-redundant protein database, or by means of a multiple sequence alignment of homologous sequences using the DNAStar program Megalign. Depending on its length, a C-terminal tail may or may not play a regulatory role in kinase function. C-terminal tail regions in the proteins of the invention can be identified using these methods. They may be present as sub-domains of the N-terminal, kinase, or C-terminal domains of the polypeptides of the invention.

Various low or high stringency hybridization conditions may be used depending upon the specificity and selectivity desired. These conditions are well-known to those skilled in the art. Under stringent hybridization conditions only highly complementary nucleic acid sequences hybridize. Preferably, such conditions prevent hybridization of nucleic acids having more than 1 or 2 mismatches out of 20 contiguous nucleotides, more preferably, such conditions prevent hybridization of nucleic acids having more than 1 or 2 mismatches out of 50 contiguous nucleotides, most preferably, such conditions prevent hybridization of nucleic acids having more than 1 or 2 mismatches out of 100 contiguous nucleotides. In some instances, the conditions may prevent hybridization of nucleic acids having more than 5 mismatches in the fall-length sequence.

By stringent hybridization assay conditions is meant hybridization assay conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart solution at 42° C. overnight; washing with 2×SSC, 0.1% SDS at 45° C.; and washing with 0.2×SSC, 0. 1% SDS at 45° C. Under some of the most stringent hybridization assay conditions, the second wash can be done with 0.1×SSC at a temperature up to 70° C. (pg. 421, Berger et al. (1987) Guide to Molecular Cloning Techniques, Meth. Enzym. vol. 152, hereby incorporated by reference herein including any figures, tables, or drawings.). However, other applications may require the use of conditions falling between these sets of conditions. Methods of determining the conditions required to achieve desired hybridizations are well-known to those with ordinary skill in the art, and are based on several factors, including but not limited to, the sequences to be hybridized and the samples to be tested.

In other preferred embodiments, the invention features isolated, enriched, or purified nucleic acid molecules encoding kinase polypeptides, further comprising a vector or promoter effective to initiate transcription in a host cell. The invention also features recombinant nucleic acid, preferably in a cell or an organism. The recombinant nucleic acid may contain a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121, or a functional derivative thereof and a vector or a promoter effective to initiate transcription in a host cell. The recombinant nucleic acid can alternatively contain a transcriptional initiation region functional in a cell, a sequence complementary to an RNA sequence encoding a kinase polypeptide and a transcriptional termination region functional in a cell. Specific vectors and host cell combinations are discussed herein. The recombinant nucleic acid can also contain the full-length sequence encoding the protein kinase, or a domain, for example.

The term “vector” relates to a single or double-stranded circular nucleic acid molecule that can be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a kinase can be inserted into a vector by cutting the vector with restriction enzymes and ligating the two pieces together.

The term “transfecting” defines a number of methods to insert a nucleic acid vector or other nucleic acid molecules into a cellular organism. These methods involve a variety of techniques, such as treating the cells with high concentrations of salt, an electric field, detergent, or DMSO to render the outer membrane or wall of the cells permeable to nucleic acid molecules of interest or use of various viral transduction strategies.

The term “promoter” as used herein, refers to nucleic acid sequence needed for gene sequence expression. Promoter regions vary from organism to organism, but are well known to persons skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

In preferred embodiments, the isolated nucleic acid comprises, consists essentially of, or consists of a nucleic acid sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121, or the corresponding full-length sequence, encodes an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ 1ID NO:241, and SEQ ID NO:242, or the corresponding full-length amino acid sequence, a functional derivative thereof, or at least 10, 20, 40, 50, 75, 100, 200, 300 or 500 contiguous amino acids of a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length sequences or derivatives thereof. The nucleic acid may be isolated from a natural source by cDNA cloning or by subtractive hybridization. The natural source may be mammalian, preferably human, blood, semen, or tissue, and the nucleic acid may be synthesized by the triester method or by using an automated DNA synthesizer.

The term “mammal” refers preferably to such organisms as mice, rats, rabbits, guinea pigs, sheep, and goats, more preferably to cats, dogs, monkeys, and apes, and most preferably to humans.

In yet other preferred embodiments, the nucleic acid is a conserved or unique region, for example those useful for: the design of hybridization probes to facilitate identification and cloning of additional polypeptides, the design of PCR probes to facilitate cloning of additional polypeptides, obtaining antibodies to polypeptide regions, and designing antisense oligonucleotides.

By “conserved nucleic acid regions”, are meant regions present on two or more nucleic acids encoding a kinase polypeptide, to which a particular nucleic acid sequence can hybridize under lower stringency conditions. Examples of lower stringency conditions suitable for screening for nucleic acid encoding kinase polypeptides are provided in Berger et al. (1987) Guide to Molecular Cloning Techniques, Meth. Enzym. vol. 152, hereby incorporated by reference herein in its entirety, including any drawings, figures, or tables. Preferably, conserved regions differ by no more than 5 out of 20 nucleotides, even more preferably 2 out of 20 nucleotides or most preferably 1 out of 20 nucleotides.

By “unique nucleic acid region” is meant a sequence present in a nucleic acid coding for a kinase polypeptide that is not present in a sequence coding for any other naturally occurring polypeptide. Such regions preferably encode 10 (preferably 25, more preferably 50, most preferably 75) or more contiguous amino acids selected from the group consisting of those set forth in SEQ ID NO:122, SEQ ID NO:123, SEQ ID NO:124, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. Preferably, the nucleic acid probe encodes a kinase polypeptide that is a fragment of the protein encoded by an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequences. The nucleic acid probe contains a nucleotide base sequence that will hybridize to a sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ D) NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121, or the corresponding full-length sequence, or a functional derivative thereof

In preferred embodiments, the nucleic acid probe hybridizes to nucleic acid encoding at least 6, 12, 75, 90, 105, 120, 150, 200, 250, 300 or 350 contiguous amino acids of a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137,.SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or functional derivatives thereof.

Methods for using the probes include detecting the presence or amount of kinase RNA in a sample by contacting the sample with a nucleic acid probe under conditions such that hybridization occurs and detecting the presence or amount of the probe bound to kinase RNA. The nucleic acid duplex formed between the probe and a nucleic acid sequence coding for a kinase polypeptide may be used in the identification of the sequence of the nucleic acid detected (Nelson et al., in Nonisotopic DNA Probe Techniques, Academic Press, San Diego, Kricka, ed., p. 275, 1992, hereby incorporated by reference herein in its entirety, including any drawings, figures, or tables). Kits for performing such methods may be constructed to include a container means having disposed therein a nucleic acid probe.

In a third aspect, the invention describes a recombinant cell or tissue comprising a nucleic acid molecule encoding a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. In such cells, the nucleic acid may be under the control of the genomic regulatory elements, or may be under the control of exogenous regulatory elements including an exogenous promoter. By “exogenous” it is meant a promoter that is not normally coupled in vivo transcriptionally to the coding sequence for the kinase polypeptides.

The polypeptide is preferably a fragment of the protein encoded by an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence. By “fragment,” is meant an amino acid sequence present in a kinase polypeptide. Preferably, such a sequence comprises at least 10, 20, 40, 50, 75, 100, 200, or 300 contiguous amino acids a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO;170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or of the corresponding full-length amino acid sequence, or a functional derivative thereof.

In a fourth aspect, the invention features an isolated, enriched, or purified kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242.

By “isolated” in reference to a polypeptide is meant a polymer of amino acids (2 or more amino acids) conjugated to each other, including polypeptides that are isolated from a natural source or that are synthesized. The isolated polypeptides of the present invention are unique in the sense that they are not found in a pure or separated state in nature. Use of the term “isolated” indicates that a naturally occurring sequence has been removed from its normal cellular environment. Thus, the sequence may be in a cell-free solution or placed in a different cellular environment. The term does not imply that the sequence is the only amino acid chain present, but that it is essentially free (about 90-95% pure at least) of non-amino acid material naturally associated with it.

By the use of the term “enriched” in reference to a polypeptide is meant that the specific amino acid sequence constitutes a significantly higher fraction (2-5 fold) of the total amino acid sequences present in the cells or solution of interest than in normal or diseased cells or in the cells from which the sequence was taken. This could be caused by a person by preferential reduction in the amount of other amino acid sequences present, or by a preferential increase in the amount of the specific amino acid sequence of interest, or by a combination of the two. However, it should be noted that enriched does not imply that there are no other amino acid sequences present, just that the relative amount of the sequence of interest has been significantly increased. The term significant here is used to indicate that the level of increase is useful to the person making such an increase, and generally means an increase relative to other amino acid sequences of about at least 2-fold, more preferably at least 5- to 10-fold or even more. The term also does not imply that there is no amino acid sequence from other sources. The other source of amino acid sequences may, for example, comprise amino acid sequence encoded by a yeast or bacterial genome, or a cloning vector such as pUC19. The term is meant to cover only those situations in which man has intervened to increase the proportion of the desired amino acid sequence.

It is also advantageous for some purposes that an amino acid sequence be in purified form. The term “purified” in reference to a polypeptide does not require absolute purity (such as a homogeneous preparation); instead, it represents an indication that the sequence is relatively purer than in the natural environment. Compared to the natural level this level should be at least 2-5 fold greater (e.g., in terms of mg/mL). Purification of at least one order of magnitude, preferably two or three orders, and more preferably four or five orders of magnitude is expressly contemplated. The substance is preferably free of contamination at a functionally significant level, for example 90%, 95%, or 99% pure.

In preferred embodiments, the kinase polypeptide is a fragment of the protein encoded by an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequences. Preferably, the kinase polypeptide contains at least 10, 20, 40, 50, 75, 100, 200, or 300 contiguous amino acids a sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or a functional derivative thereof.

In preferred embodiments, the kinase polypeptide comprises an amino acid sequence having (a) an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; () an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, except that it lacks one or more, but not all, of a domain selected from the group consisting of an N-terminal domain, a catalytic domain, a C-terminal domain, a coiled-coil structure region, a proline-rich region, a spacer region, an insert, and a C-terminal tail; (c) an amino acid sequence of a domain of a polypeptide selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 where the domain is selected from the group consisting of an N-terminal domain, a catalytic domain, a C-terminal domain, a coiled-coil structure region, a proline-rich region, a spacer region, an insert, and a C-terminal tail; or (d) an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ II NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, except that it lacks one or more, but not all, of the domains selected from the group consisting of a C-terminal domain, a catalytic domain, an N-terminal domain, a spacer region, a proline-rich region, a coiled-coil structure region, an insert, and a C-terminal tail. (The domain demarcations of the polypeptides of the invention are indicated in Table 2 by reference to the kinase domain.)

The polypeptide can be isolated from a natural source by methods well-known in the art. The natural source may be mammalian, preferably human, blood, semen, or tissue, and the polypeptide may be synthesized using an automated polypeptide synthesizer. The isolated, enriched, or purified kinase polypeptide is preferably selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242A.

In some embodiments the invention includes a recombinant kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. By “recombinant kinase polypeptide” is meant a polypeptide produced by recombinant DNA techniques such that it is distinct from a naturally occurring polypeptide either in its location (e.g., present in a different cell or tissue than found in nature), purity or structure. Generally, such a recombinant polypeptide will be present in a cell in an amount different from that normally observed in nature.

In a fifth aspect, the invention features an antibody (e.g., a monoclonal or polyclonal antibody) having specific binding affinity to a kinase polypeptide or a kinase polypeptide domain or fragment where the polypeptide is selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ I) NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. In preferred embodiments, the antibody binds specifically to domains of kinase polypeptides, that are defined supra.

By “specific binding affinity” is meant that the antibody binds to the target kinase polypeptide with greater affinity than it binds to other polypeptides under specified conditions. Antibodies or antibody fragments are polypeptides that contain regions that can bind other polypeptides. The term “specific binding affinity” describes an antibody that binds to a kinase polypeptide with greater affinity than it binds to other polypeptides under specified conditions.

The term “polyclonal” refers to antibodies that are heterogenous populations of antibody molecules derived from the sera of animals immunized with an antigen or an antigenic functional derivative thereof For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art (Kohler et al., Nature 256:495-497, 1975, and U.S. Pat. No. 4,376,110, both of which are hereby incorporated by reference herein in their entirety including any figures, tables, or drawings).

The term “antibody fragment” refers to a portion of an antibody, often the hyper variable region and portions of the surrounding heavy and light chains, that displays specific binding affinity for a particular molecule. A hyper variable region is a portion of an antibody that physically binds to the polypeptide target.

Antibodies or antibody fragments having specific binding affinity to a kinase polypeptide or domains of a kinase polypeptide of the invention may be used in methods for detecting the presence and/or amount of kinase polypeptide in a sample by probing the sample with the antibody under conditions suitable for kinase-antibody immunocomplex formation and detecting the presence and/or amount of the antibody conjugated to the kinase polypeptide. Diagnostic kits for performing such methods may be constructed to include antibodies or antibody fragments specific for the kinase as well as a conjugate of a binding partner of the antibodies or the antibodies themselves.

An antibody or antibody fragment with specific binding affinity to a kinase polypeptide of the invention can be isolated, enriched, or purified from a prokaryotic or eukaryotic organism. Routine methods known to those skilled in the art enable production of antibodies or antibody fragments, in both prokaryotic and eukaryotic organisms. Purification, enrichment, and isolation of antibodies, which are polypeptide molecules, are described above.

Antibodies having specific binding affinity to a kinase polypeptide of the invention may be used in methods for detecting the presence and/or amount of kinase polypeptide in a sample by contacting the sample with the antibody under conditions such that an immunocomplex forms and detecting the presence and/or amount of the antibody conjugated to the kinase polypeptide. Diagnostic kits for performing such methods may be constructed to include a first container containing the antibody and a second container having a conjugate of a binding partner of the antibody and a label, such as, for example, a radioisotope. The diagnostic kit may also include notification of an FDA approved use and instructions therefor.

In a sixth aspect, the invention features a hybridoma which produces an antibody having specific binding affinity to a kinase polypeptide or a kinase polypeptide domain, where the polypeptide is selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; and where the domains are defined as above. By “hybridoma” is meant an immortalized cell line that is capable of secreting an antibody, for example an antibody to a kinase of the invention. In preferred embodiments, the antibody to the kinase comprises a sequence of amino acids that is able to specifically bind a kinase polypeptide of the invention.

In a seventh aspect, the invention features a kinase polypeptide binding agent able to bind to a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. The binding agent is preferably a purified antibody that recognizes an epitope present on a kinase polypeptide of the invention. Other binding agents include molecules that bind to kinase polypeptides and analogous molecules that bind to a kinase polypeptide. Such binding agents may be identified by using assays that measure kinase binding partner activity, such as those that measure PDGFR activity.

The invention also features a method for screening for human cells containing a kinase polypeptide of the invention or an equivalent sequence. The method involves identifying the novel polypeptide in human cells using techniques that are routine and standard in the art, such as those described herein for identifying the kinases of the invention (e.g., cloning, Southern or Northern blot analysis, in situ hybridization, PCR amplification, etc.).

In an eighth aspect, the invention features methods for identifying a substance that modulates kinase activity comprising the steps of: (a) contacting a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 with a test substance; (b) measuring the activity of said polypeptide; and (c) determining whether said substance modulates the activity of said polypeptide.

The term “modulates” refers to the ability of a compound to alter the function of a kinase of the invention. A modulator preferably activates or inhibits the activity of a kinase of the invention.

The term “activates” refers to increasing the cellular activity of the kinase. The term inhibit refers to decreasing the cellular activity of the kinase. Kinase activity is preferably the interaction with a natural binding partner.

The term “modulates” also refers to altering the function of kinases of the invention by increasing or decreasing the probability that a complex forms between the kinase and a natural binding partner. A modulator preferably increases the probability that such a complex forms between the kinase and the natural binding partner, more preferably increases or decreases the probability that a complex forms between the kinase and the natural binding partner depending on the concentration of the compound exposed to the kinase, and most preferably decreases the probability that a complex forms between the kinase and the natural binding partner.

The term “complex” refers to an assembly of at least two molecules bound to one another. Signal transduction complexes often contain at least two protein molecules bound to one another. For instance, a protein tyrosine receptor protein kinase, GRB2, SOS, RAF, and RAS assemble to form a signal transduction complex in response to a mitogenic ligand.

The term “natural binding partner” refers to polypeptides, lipids, small molecules, or nucleic acids that bind to kinases in cells. A change in the interaction between a kinase and a natural binding partner can manifest itself as an increased or decreased probability that the interaction forms, or an increased or decreased concentration of kinase/natural binding partner complex.

The term “contacting” as used herein refers to mixing a solution comprising the test compound with a liquid medium bathing the cells of the methods. The solution comprising the compound may also comprise another component, such as dimethyl sulfoxide (DMSO), which facilitates the uptake of the test compound or compounds into the cells of the methods. The solution comprising the test compound may be added to the medium bathing the cells by utilizing a delivery apparatus, such as a pipet-based device or syringe-based device.

In a ninth aspect, the invention features methods for identifying a substance that modulates kinase activity in a cell comprising the steps of: (a) expressing a kinase polypeptide in a cell, wherein said polypeptide is selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242; (b) adding a test substance to said cell; and (c) monitoring a change in cell phenotype or the interaction between said polypeptide and a natural binding partner.

The term “expressing” as used herein refers to the production of kinases of the invention from a nucleic acid vector containing kinase genes within a cell. The nucleic acid vector is transfected into cells using well known techniques in the art as described herein.

In a tenth aspect, the invention provides methods for treating a disease or abnormal condition by administering to a patient in need of such treatment a substance that modulates the activity of a polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242. Preferably, the disease is selected from the group consisting of immune-related diseases and disorders, cardiovascular disease, neurodegenerative disorders, and cancer. Also included are metabolic disorders, such as diabetes mellitus, and reproductive disorders, such as infertility.

Preferably, the disease or disorder is selected from the group consisting of rheumatoid arthritis, artherosclerosis, autoimmune disorders, and organ transplantation. Preferably the disease or disorder is selected from the group consisting of immune-related diseases and disorders, myocardial infarction, cardiomyopathies, stroke, renal failure, and oxidative stress-related neurodegenerative disorders. Most preferably, the immune-related diseases and disorders are selected from the group consisting of rheumatoid arthritis, chronic inflammatory bowel disease, chronic inflammatory pelvic disease, multiple sclerosis, asthma, osteoarthritis, psoriasis, atherosclerosis, rhinitis, autoimmunity, and organ transplantation.

Substances useful for treatment of disorders or diseases preferably show positive results in one or more in vitro assays for an activity corresponding to treatment of the disease or disorder in question Substances that modulate the activity of the polypeptides preferably include, but are not limited to, antisense oligonucleotides and inhibitors of protein kinases.

The term “preventing” refers to decreasing the probability that an organism contracts or develops an abnormal condition.

The term “treating” refers to having a therapeutic effect and at least partially alleviating or abrogating an abnormal condition in the organism.

The term “therapeutic effect” refers to the inhibition or activation factors causing or contributing to the abnormal condition. A therapeutic effect relieves to some extent one or more of the symptoms of the abnormal condition. In reference to the treatment of abnormal conditions, a therapeutic effect can refer to one or more of the following: (a) an increase in the proliferation, growth, and/or differentiation of cells; (b) inhibition (i.e., slowing or stopping) of cell death; (c) inhibition of degeneration; (d) relieving to some extent one or more of the symptoms associated with the abnormal condition; and (e) enhancing the function of the affected population of cells. Compounds demonstrating efficacy against abnormal conditions can be identified as described herein.

The term “abnormal condition” refers to a function in the cells or tissues of an organism that deviates from their normal functions in that organism. An abnormal condition can relate to cell proliferation, cell differentiation or cell survival. An abnormal condition may also include irregularities in cell cycle progression, i.e., irregularities in normal cell cycle progression through mitosis and meiosis.

Abnormal cell proliferative conditions include cancers such as fibrotic and mesangial disorders, abnormal angiogenesis and vasculogenesis, wound healing, psoriasis, diabetes mellitus, and inflammation.

Abnormal differentiation conditions include, but are not limited to neurodegenerative disorders, slow wound healing rates, and slow tissue grafting healing rates.

Abnormal cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

The tern “aberration”, in conjunction with the function of a kinase in a signal transduction process, refers to a kinase that is over- or under-expressed in an organism, mutated such that its catalytic activity is lower or higher than wild-type protein kinase activity, mutated such that it can no longer interact with a natural binding partner, is no longer modified by another protein kinase or protein phosphatase, or no longer interacts with a natural binding partner.

The term “administering” relates to a method of incorporating a compound into cells or tissues of an organism. The abnormal condition can be prevented or treated when the cells or tissues of the organism exist within the organism or outside of the organism. Cells existing outside the organism can be maintained or grown in cell culture dishes. For cells harbored within the organism, many techniques exist in the art to administer compounds, including (but not limited to) oral parenteral, dermal, injection, and aerosol applications. For cells outside of the organism, multiple techniques exist in the art to administer the compounds, including (but not limited to) cell microinjection techniques, transformation techniques, and carrier techniques.

The abnormal condition can also be prevented or treated by administering a compound to a group of cells having an aberration in a signal transduction pathway to an organism. The effect of administering a compound on organism function can then-be monitored. The organism is preferably a mouse, rat, rabbit, guinea pig, or goat, more preferably a monkey or ape, and most preferably a human.

In an eleventh aspect, the invention features methods for detection the expression of a polypeptide in a sample as a diagnostic tool for diseases or disorders, wherein the method comprises the steps of: (a) contacting the sample with a nucleic acid probe which hybridizes under hybridization assay conditions to a nucleic acid target region of a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, said probe comprising the nucleic acid sequence encoding the polypeptide, fragments thereof, and the complements of the sequences and fragments; and (b) detecting the presence or amount of the probe:target region hybrid as an indication of the disease.

In preferred embodiments of the invention, the disease or disorder is selected from the group consisting of rheumatoid arthritis, artherosclerosis, autoimmune disorders, organ transplantation, myocardial infarction, cardiomyopathies, stroke, renal failure, oxidative stress-related neurodegenerative disorders, metabolic disorder including diabetes, reproductive disorders including infertility, and cancer.

The kinase “target region” is a nucleotide base sequence selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:l 1, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:l09, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121, or the corresponding full-length sequences, a functional derivative thereof, or a fragment thereof to which the nucleic acid probe will specifically hybridize. Specific hybridization indicates that in the presence of other nucleic acids the probe only hybridizes detectably with the kinase of the invention's target region. Putative target regions can be identified by methods well known in the art consisting of alignment and comparison of the most closely related sequences in the database.

In preferred embodiments the nucleic acid probe hybridizes to a kinase target region encoding at least 6, 12, 75, 90, 105, 120, 150, 200, 250, 300 or 350 contiguous amino acids of the sequence set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or the corresponding full-length amino acid sequence, or a functional derivative thereof. Hybridization conditions should be such that hybridization occurs only with the kinase genes in the presence of other nucleic acid molecules. Under stringent hybridization conditions only highly complementary nucleic acid sequences hybridize. Preferably, such conditions prevent hybridization of nucleic acids having more than 1 or 2 mismatches out of 20 contiguous nucleotides. Such conditions are defined supra.

Hybridization conditions should be such that hybridization occurs only with the genes in the presence of other nucleic acid molecules. Under stringent hybridization conditions only highly complementary nucleic acid sequences hybridize. Preferably, such conditions prevent hybridization of nucleic acids having 1 or 2 mismatches out of 20 contiguous nucleotides. Such conditions are defined supra.

The diseases for which detection of kinase genes in a sample could be diagnostic include diseases in which kinase nucleic acid (DNA and/or RNA) is amplified in comparison to normal cells. By “amplification” is meant increased numbers of kinase DNA or RNA in a cell compared with normal cells. In normal cells, kinases are typically found as single copy genes. In selected diseases, the chromosomal location of the kinase genes may be amplified, resulting in multiple copies of the gene, or amplification. Gene amplification can lead to amplification of kinase RNA, or kinase RNA can be amplified in the absence of kinase DNA amplification.

“Amplification” as it refers to RNA can be the detectable presence of kinase RNA in cells, since in some normal cells there is no basal expression of kinase RNA. In other normal cells, a basal level of expression of kinase exists, therefore in these cases amplification is the detection of at least 1-2-fold, and preferably more, kinase RNA, compared to the basal level.

The diseases that could be diagnosed by detection of kinase nucleic acid in a sample preferably include cancers. The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

Another aspect of the invention involves a method of agonizing (stimulating) or antagonizing a target of the invention and a natural binding partner associated activity in a mammal comprising administering to said mammal an agonist or antagonist to one of the above disclosed polypeptides in an amount sufficient to effect said agonism or antagonism. A method of treating diseases in a mammal with an agonist or antagonist of the protein of the present invention activity comprising administering the agonist or antagonist to a mammal in an amount sufficient to agonize or antagonize associated functions is also encompassed in the present application.

In an effort to discover novel treatments for diseases, biomedical researchers and chemists have designed, synthesized, and tested molecules that inhibit the function of protein polypeptides. Some small organic molecules form a class of compounds that modulate the function of protein polypeptides. Examples of molecules that have been reported to inhibit the function of protein kinases include, but are not limited to, bis monocyclic, bicyclic or heterocyclic aryl compounds (PCT WO 92/20642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP application No. 0,566,266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al, all of which are incorporated by reference herein, including any drawings.

Compounds that can traverse cell membranes and are resistant to acid hydrolysis are potentially advantageous as therapeutics as they can become highly bioavailable after being administered orally to patients. However, many of these protein inhibitors only weakly inhibit function. In addition, many inhibit a variety of protein kinases and will therefore cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar groups including hydroxylated alkyl, phosphate, and ether substituents. U.S. patent application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187) and Ser. No. 08/485,323, filed Jun. 7,1995, entitled “Benzylidene-Z-Indoline Compounds for the Treatment of Disease” by Tang et al. (Lyon & Lyon Docket No. 223/298) and International Patent Publication WO 96/22976, published Aug. 1, 1996 by Ballinari et al., all of which are incorporated herein by reference in their entirety, including any drawings, describe indolinone chemical libraries of indolinone compounds harboring other bicyclic moieties as well as monocyclic moieties fused to the oxindole ring. Applications Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187), Ser. No. 08/485,323, filed Jun. 7, 1995, entitled “Benzylidene-Z-Indoline Compounds for the Treatment of Disease” by Tang et al. (Lyon & Lyon Docket No. 223/298), and WO 96/22976, published Aug. 1, 1996 by Ballinari et al. teach methods of indolinone synthesis, methods of testing the biological activity of indolinone compounds in cells, and inhibition patterns of indolinone derivatives, both of which are incorporated by reference herein, including any drawings.

Other examples of substances capable of modulating kinase activity include, but are not limited to, tyrphostins, quinazolines, quinoxolines, and quinolines. The quinazolines, tyrphostins, quinolines, and quinoxolines referred to above include well known compounds such as those described in the literature. For example, representative publications describing quinazolines include Barker et al., EPO Publication No. 0 520 722 Al; Jones et al., U.S. Pat. No. 4,447,608; Kabbe et al., U.S. Pat. No. 4,757,072; Kaul and Vougioukas, U.S. Pat. No. 5,316,553; Kreighbaum and Comer, U.S. Pat. No. 4,343,940; Pegg and Wardleworth, EPO Publication No. 0 562 734 Al; Barker et al., Proc. of Am. Assoc. for Cancer Research 32:327 (1991); Bertino, J. R., Cancer Research 3:293-304 (1979); Bertino, J. R, Cancer Research 9(2 part 1):293-304 (1979); Curtin et al., Br. J. Cancer 53:361-368 (1986); Fernandes et al., Cancer Research 43:1117-1123 (1983); Ferris et al. J. Org. Chem. 44(2):173-178; Fry et al., Science 265:1093-1095 (1994); Jackman et al., Cancer Research 51:5579-5586 (1981); Jones et al. J. Med. Chem. 29(6):1114-1118; Lee and Skibo, Biochemistry 26(23):7355-7362 (1987); Lemus et al., J. Org. Chem. 54:3511-3518 (1989); Ley and Seng, Synthesis 1975:415-522 (1975); Maxwell et al., Magnetic Resonance in Medicine 17:189-196 (1991); Mini et al., Cancer Research 45:325-330 (1985); Phillips and Castle, J. Heterocyclic Chem. 17(19):1489-1596 (1980); Reece et al., Cancer Research 47(11):2996-2999 (1977); Sculier et al., Cancer Immunol. and Immunother. 23:A65 (1986); Sikora et al., Cancer Letters 23:289-295 (1984); and Sikora et al., Analytical Biochem. 172:344-355 (1988), all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., J. Med. Chem. 37:2627-2629 (1994); MaGuire, J. Med. Chem. 37:2129-2131 (1994); Burke et al., J. Med. Chem. 36:425-432 (1993); and Burke et al. BioOrganic Med. Chem. Letters 2:1771-1774 (1992), all of which are incorporated by reference in their entirety, including any drawings.

Tyrphostins are described in Allen et al., Clin. Exp. Immunol. 91:141-156 (1993); Anafi et al., Blood 82:12:3524-3529 (1993); Baker et al., J. Cell Sci. 102:543-555 (1992); Bilderet al., Amer. Physiol. Soc. pp. 6363-6143:C721-C730 (1991); Brunton et al., Proceedings of Amer. Assoc. Cancer Rsch. 33:558 (1992); Bryckaert et al., Experimental Cell Research 199:255-261 (1992); Dong et al., J. Leukocyte Biology 53:53-60 (1993); Dong et al., J. Immunol. 151(5):2717-2724 (1993); Gazit et al., J. Med. Chem. 32:2344-2352 (1989); Gazit et al., “J. Med. Chem. 36:3556-3564 (1993); Kaur et al., Anti-Cancer Drugs 5:213-222 (1994); Kaur et al., King et al., Biochem. J. 275:413418 (1991); Kuo et al., Cancer Letters 74:197-202 (1993); Levitzki, A., The FASEB J. 6:3275-3282 (1992); Lyall et al., J. Biol. Chem. 264:14503-14509 (1989); Peterson et al., The Prostate 22:335-345 (1993); Pillemer et al., Int. J. Cancer 50:80-85 (1992); Posner et al., Molecular Pharmacology 45:673-683 (1993); Rendu et al., Biol. Pharmacology 44(5):881-888 (1992); Sauro and Thomas, Life Sciences 53:371-376 (1993); Sauro and Thomas, J. Pharm. and Experimental Therapeutics 267(3):119-1125 (1993); Wolbring et al., J. Biol. Chem. 269(36):22470-22472 (1994); and Yoneda et al., Cancer Research 51:4430-4435 (1991); all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

Methods of Treating a Disease (Enablement—i.e., Dosing)

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures or tables. Those skilled in the art will appreciate that such descriptions are applicable to the present invention and can be easily adapted to it.

The proper dosage depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be deter-mined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, Journal of American Veterinary Medical Assoc., 202:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

In a final aspect, the invention features a method for detection of a kinase polypeptide in a sample as a diagnostic tool for a disease or disorder, wherein the method comprises: (a) comparing a nucleic acid target region encoding the kinase polypeptide in a sample, where the kinase polypeptide is selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or one or more fragments thereof, with a control nucleic acid target region encoding the kinase polypeptide, or one or more fragments thereof; and (b) detecting differences in sequence or amount between the target region and the control target region, as an indication of the disease or disorder. Preferably, the disease or disorder is selected from the group consisting of immune-related diseases and disorders, organ transplantation, myocardial infarction, cardiovascular disease, stroke, renal failure, oxidative stress-related neurodegenerative disorders, and cancer. Immune-related diseases and disorders include, but are not limited to, those discussed previously.

The term “comparing” as used herein refers to identifying discrepancies between the nucleic acid target region isolated from a sample, and the control nucleic acid target region. The discrepancies can be in the nucleotide sequences, e.g. insertions, deletions, or point mutations, or in the amount of a given nucleotide sequence. Methods to determine these discrepancies in sequences are well-known to one of ordinary skill in the art. The “control” nucleic acid target region refers to the sequence or amount of the sequence found in normal cells, e.g. cells that are not diseased as discussed previously.

The term also includes anti-sense molecules drawn thereto.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. For example, in some instances the nucleotide sequence of particular kinase polypeptides may not be part of a preferred embodiment.

The summary of the invention described above is not limiting and other features and advantages of the invention will be apparent from the following detailed description of the invention, and from the claims.

### BRIEF DESCRIPTION OF THE FIGURES

FIGS. 1A to 1BB shows the amino acid sequences of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242.

FIGS. 2A to 2MMMM shows the nucleic acid sequences of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121.

### DETAILED DESCRIPTION OF THE INVENTION

The present invention relates in part to kinase polypeptides, nucleic acids encoding such polypeptides, cells containing such nucleic acids, antibodies to such polypeptides, assays utilizing such polypeptides, and methods relating to all of the foregoing. The present invention is based upon the isolation and characterization of new kinase polypeptides. The polypeptides and nucleic acids may be produced using well-known and standard synthesis techniques when given the sequences presented herein.

I. The Nucleic Acids of the Invention

Included within the scope of this invention are the functional equivalents of the herein-described isolated nucleic acid molecules. The degeneracy of the genetic code permits substitution of certain codons by other codons that specify the same amino acid and hence would give rise to the same protein. The nucleic acid sequence can vary substantially since, with the exception of methionine and tryptophan, the known amino acids can be coded for by more than one codon. Thus, portions or all of the kinase genes of the invention could be synthesized to give a nucleic acid sequence significantly different from one selected from the group consisting of those set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:119, SEQ ID NO:120, and SEQ ID NO:121. The encoded amino acid sequence thereof would, however, be preserved.

In addition, the nucleic acid sequence may comprise a nucleotide sequence which results from the addition, deletion or substitution of at least one nucleotide to the 5′-end and/or the 3′-end of the nucleic acid sequence shown in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ D NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, SEQ ID NO:67, SEQ ID NO:68, SEQ ID NO:69, SEQ ID NO:70, SEQ ID NO:71, SEQ ID NO:72, SEQ ID NO:73, SEQ ID NO:74, SEQ ID NO:75, SEQ ID NO:76, SEQ ID NO:77, SEQ ID NO:78, SEQ ID NO:79, SEQ ID NO:80, SEQ ID NO:81, SEQ ID NO:82, SEQ ID NO:83, SEQ ID NO:84, SEQ ID NO:85, SEQ ID NO:86, SEQ ID NO:87, SEQ ID NO:88, SEQ ID NO:89, SEQ ID NO:90, SEQ ID NO:91, SEQ ID NO:92, SEQ ID NO:93, SEQ ID NO:94, SEQ ID NO:95, SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, SEQ ID NO:107, SEQ ID NO:108, SEQ ID NO:109, SEQ ID NO:110, SEQ ID NO:111, SEQ ID NO:112, SEQ ID NO:113, SEQ ID NO:114, SEQ ID NO:115, SEQ ID NO:116, SEQ ID NO:117, SEQ ID NO:118, SEQ ID NO:1 19, SEQ ID NO:120, and SEQ ID NO:121, or a derivative thereof. Any nucleotide or polynucleotide may be used in this regard, provided that its addition, deletion or substitution does not alter the amino acid sequence of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, that is encoded by the nucleotide sequence. For example, the present invention is intended to include any nucleic acid sequence resulting from the addition of ATG as an initiation codon at the 5′-end of the inventive nucleic acid sequence or its derivative, or from the addition of TTA, TAG or TGA as a termination codon at the 3′-end of the inventive nucleotide sequence or its derivative. Moreover, the nucleic acid molecule of the present invention may, as necessary, have restriction endonuclease recognition sites added to its 5′-end and/or 3′-end.

Such functional alterations of a given nucleic acid sequence afford an opportunity to promote secretion and/or processing of heterologous proteins encoded by foreign nucleic acid sequences fused thereto, for example. All variations of the nucleotide sequence of the kinase genes of the invention and fragments thereof permitted by the genetic code are, therefore, included in this invention.

Further, it is possible to delete codons or to substitute one or more codons with codons other than degenerate codons to produce a structurally modified polypeptide, but one which has substantially the same utility or activity as the polypeptide produced by the unmodified nucleic acid molecule. As recognized in the art, the two polypeptides are functionally equivalent, as are the two nucleic acid molecules that give rise to their production, even though the differences between the nucleic acid molecules are not related to the degeneracy of the genetic code. This is discussed further in the “Functional Derivatives” section, herein.

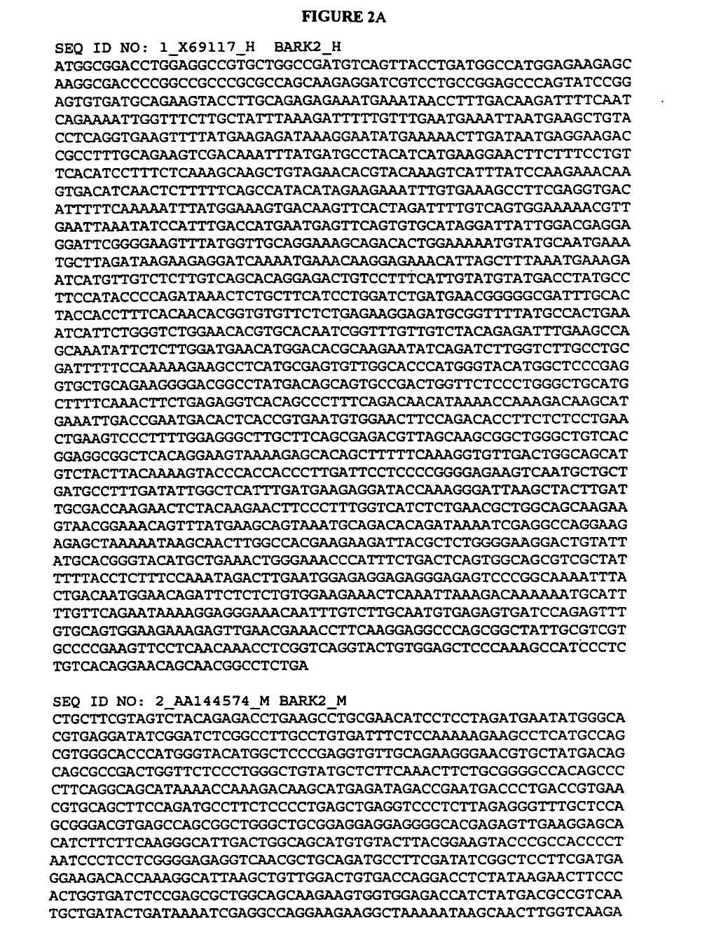

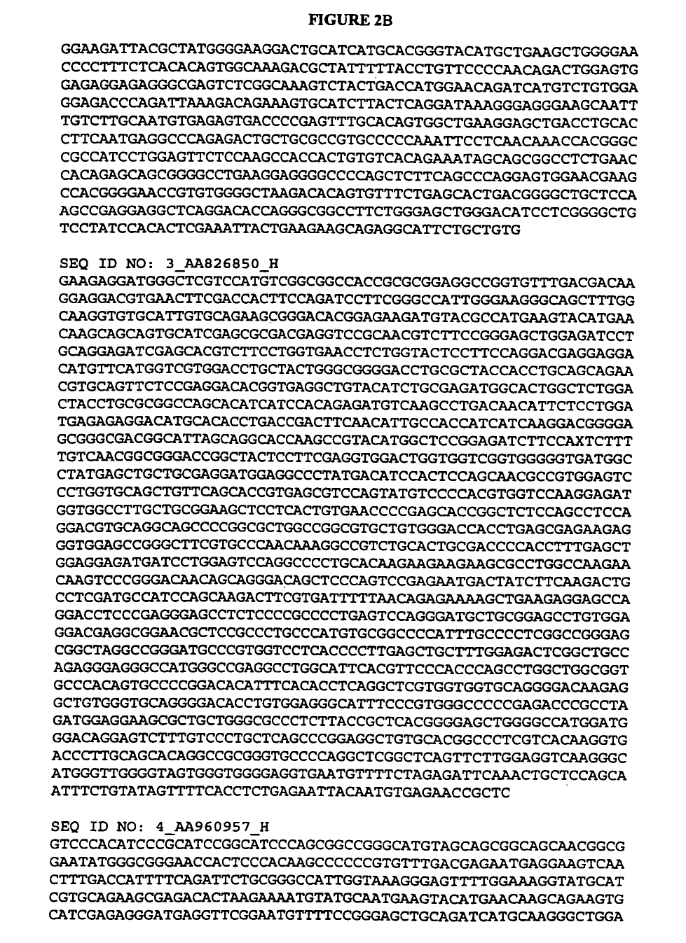

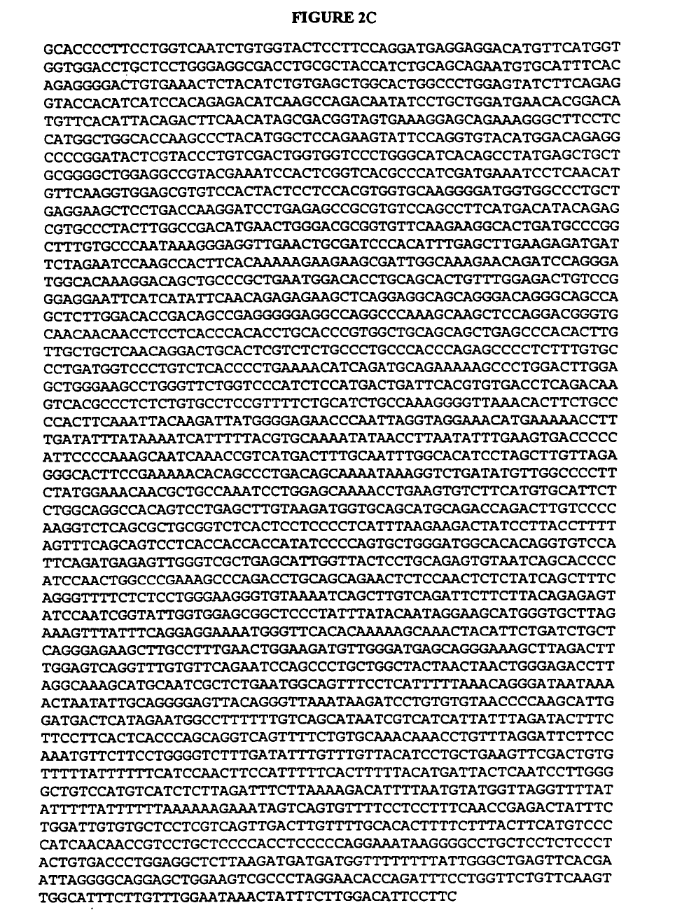

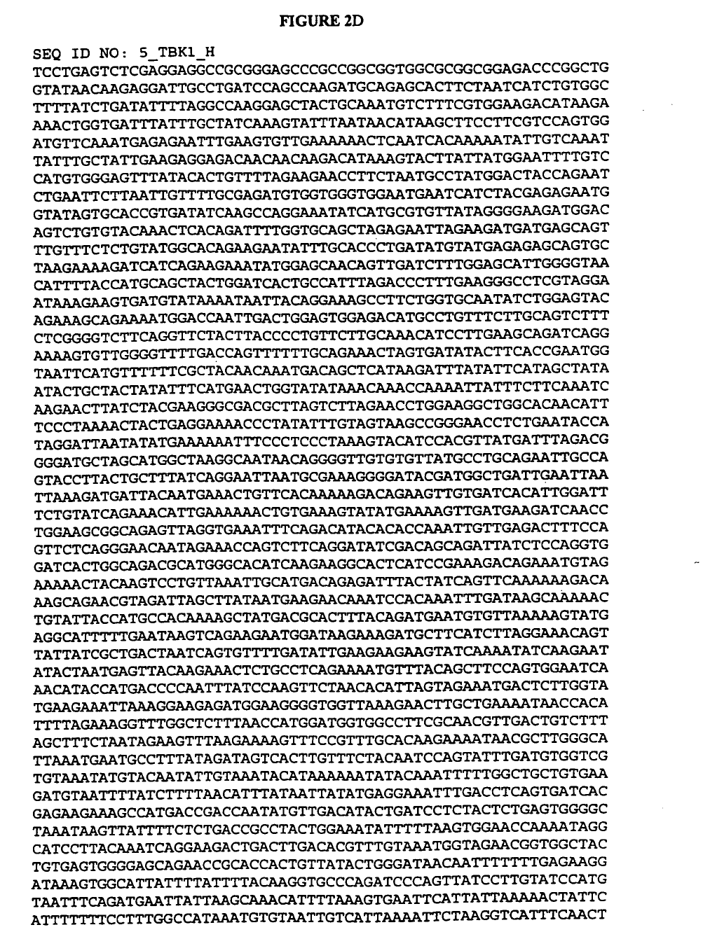

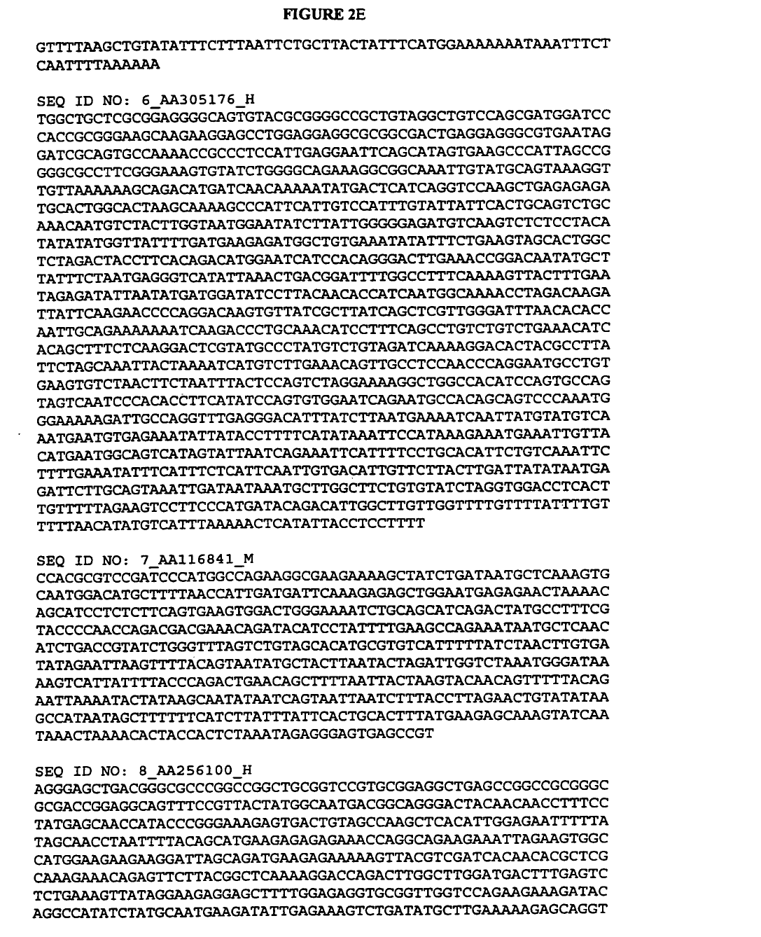

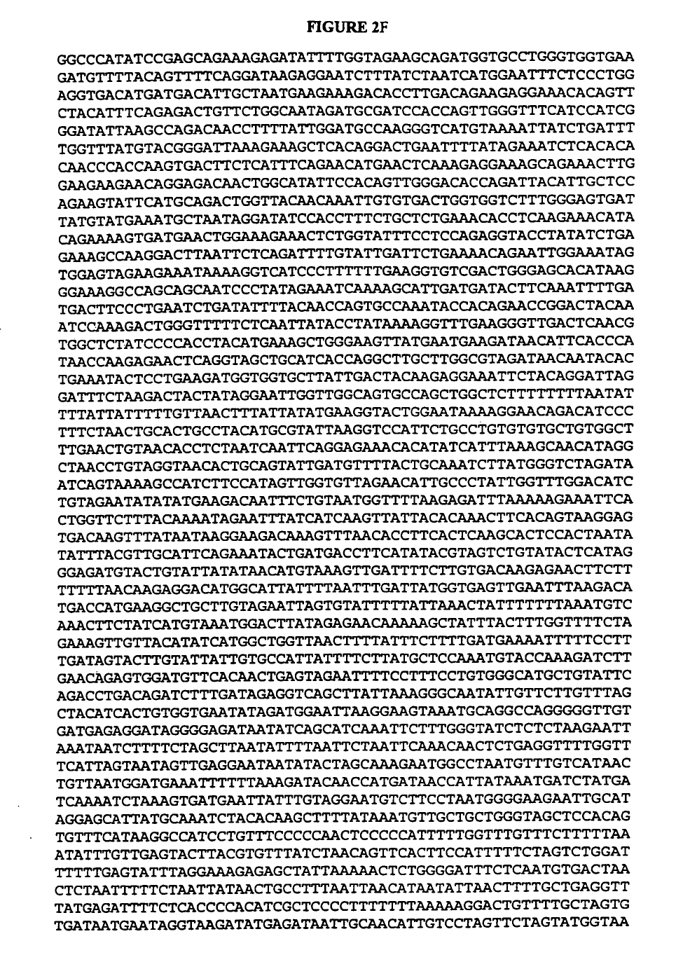

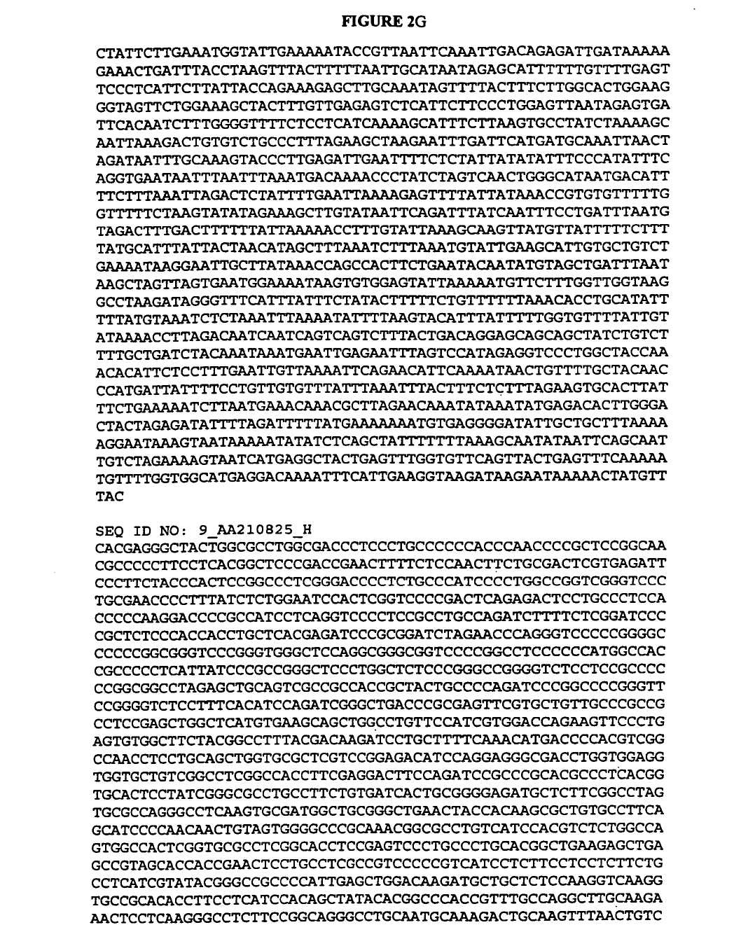

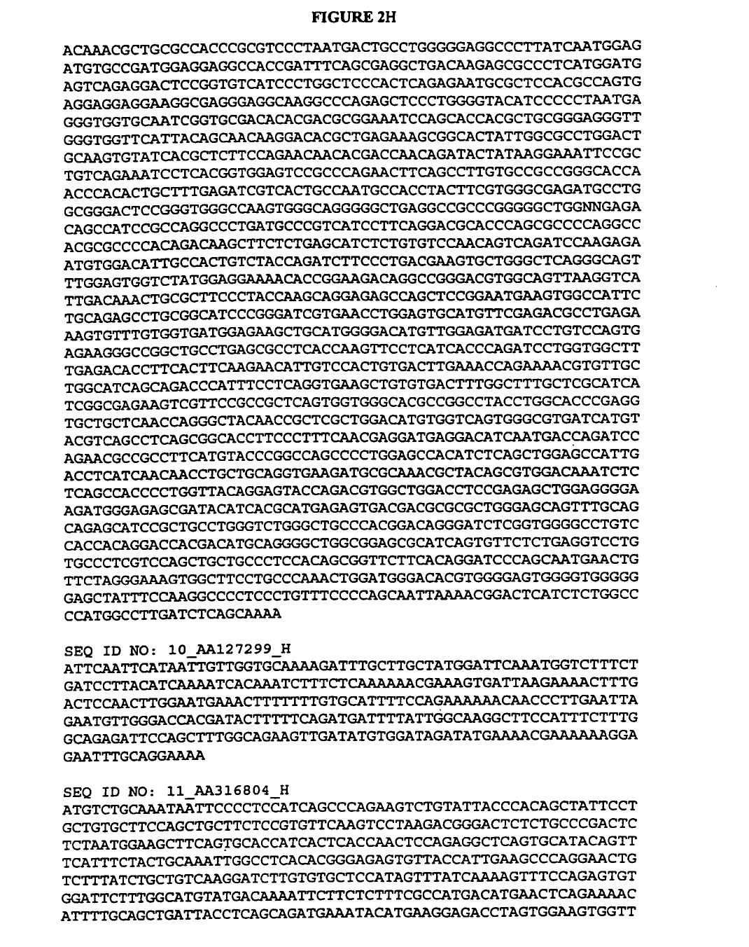

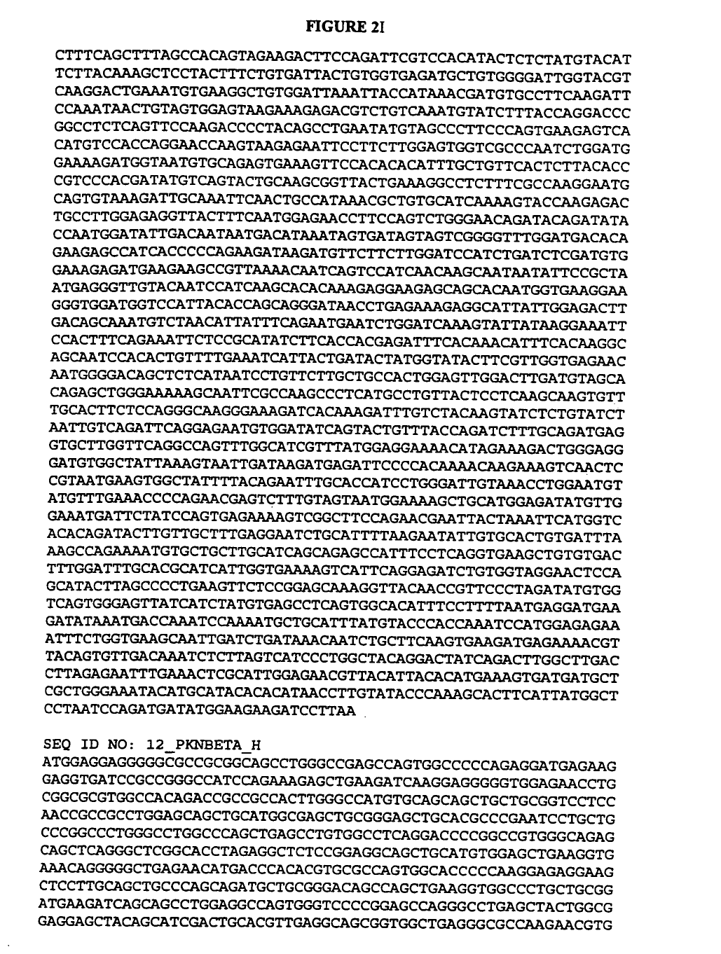

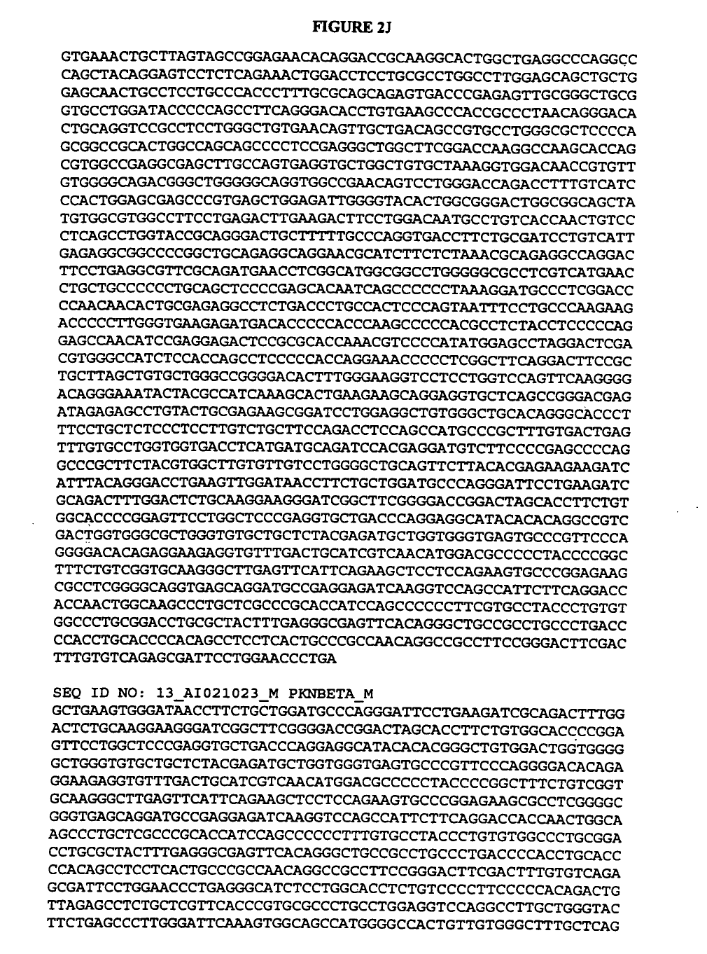

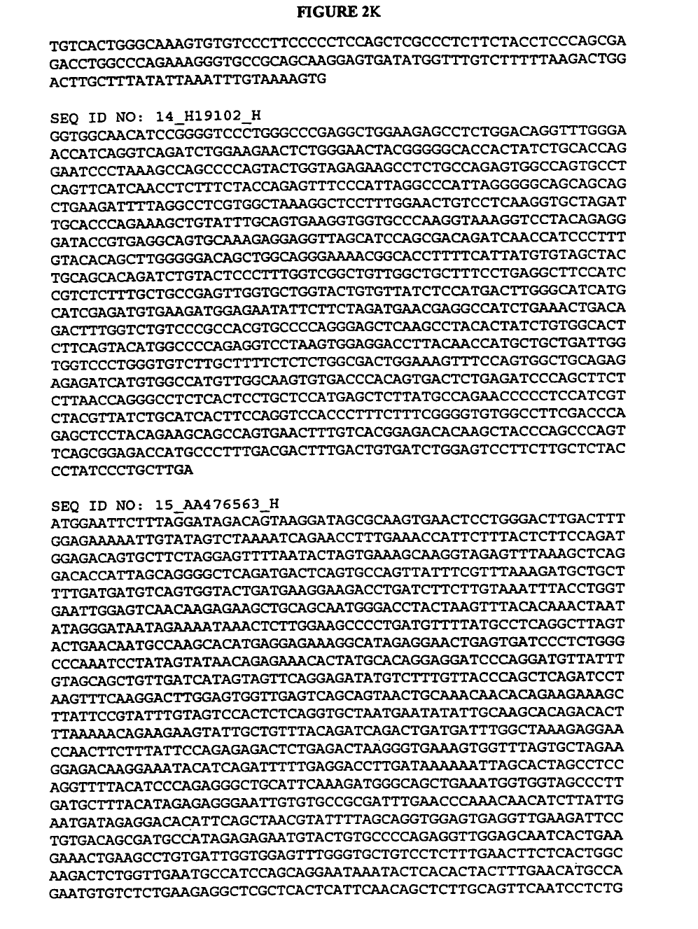

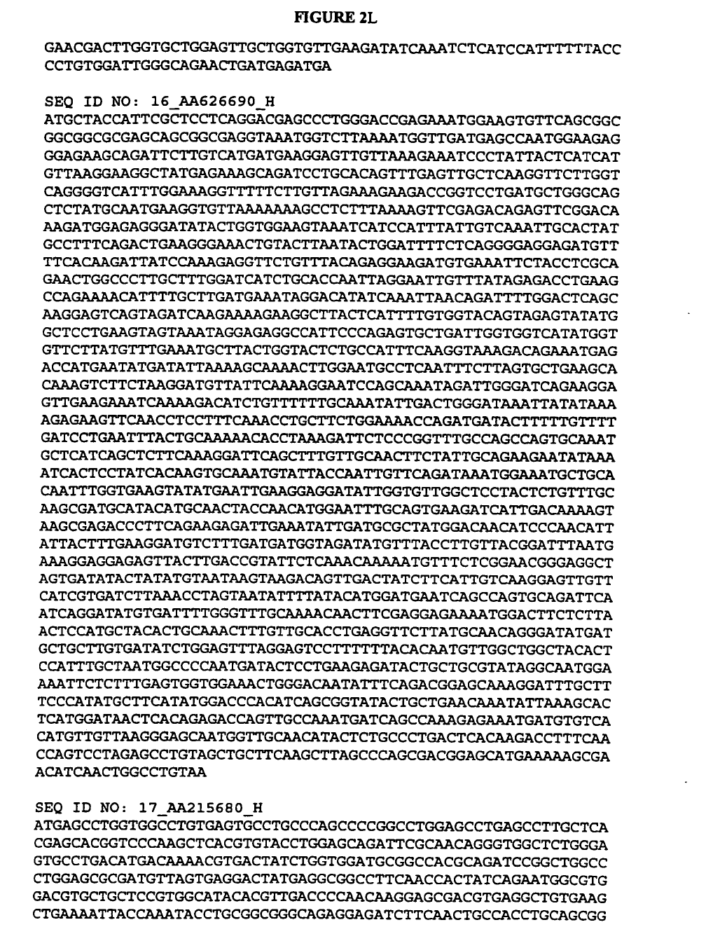

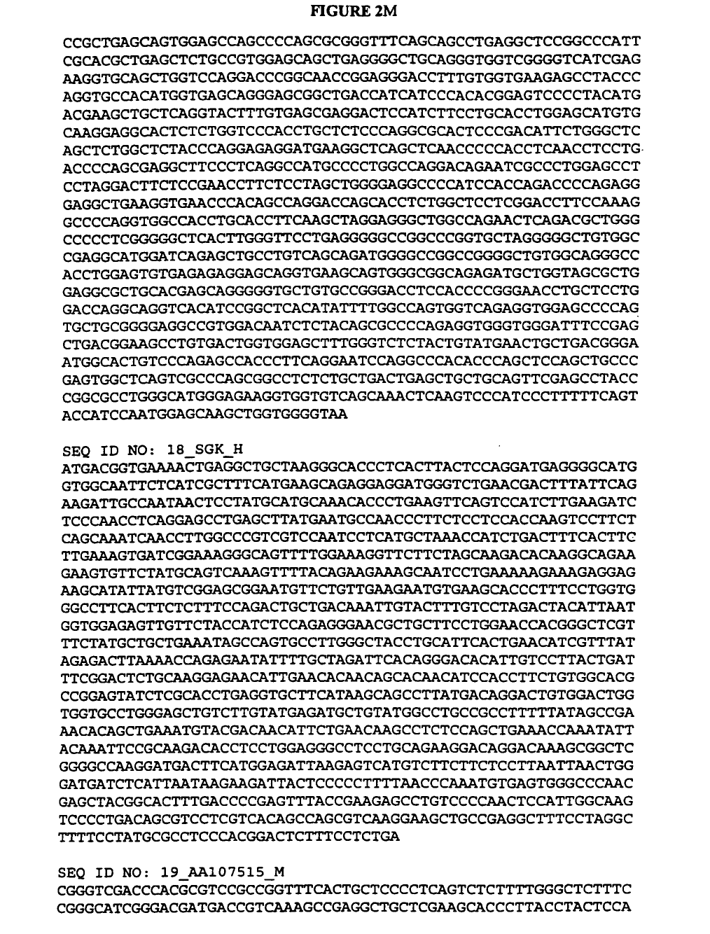

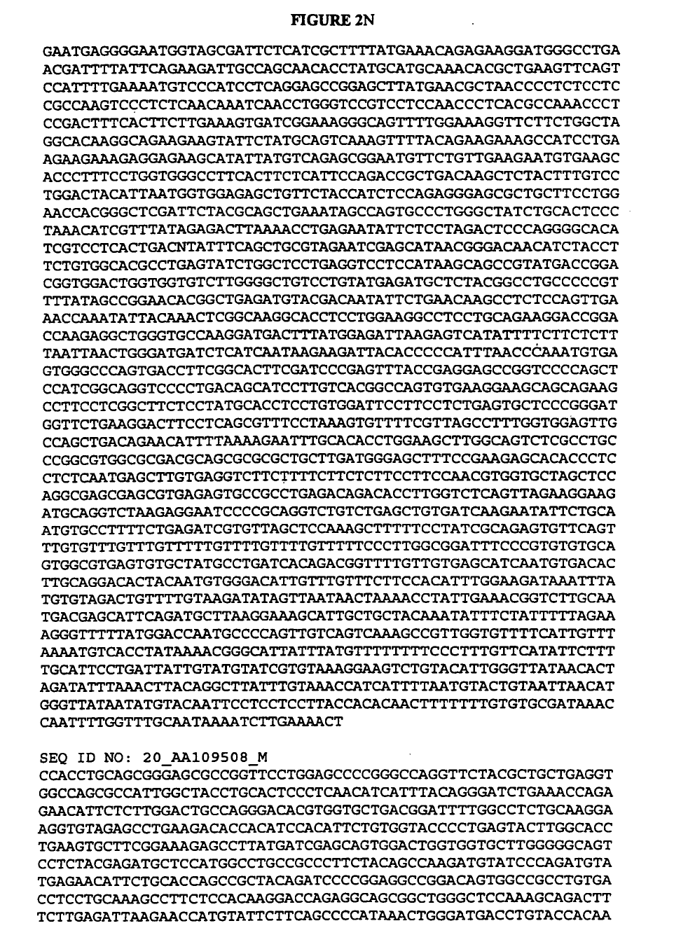

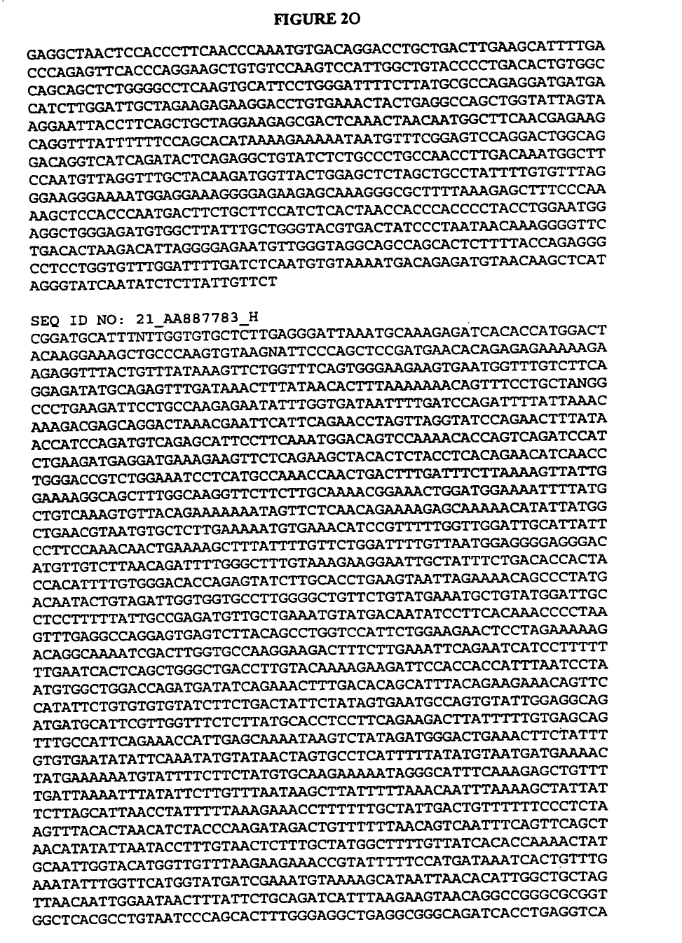

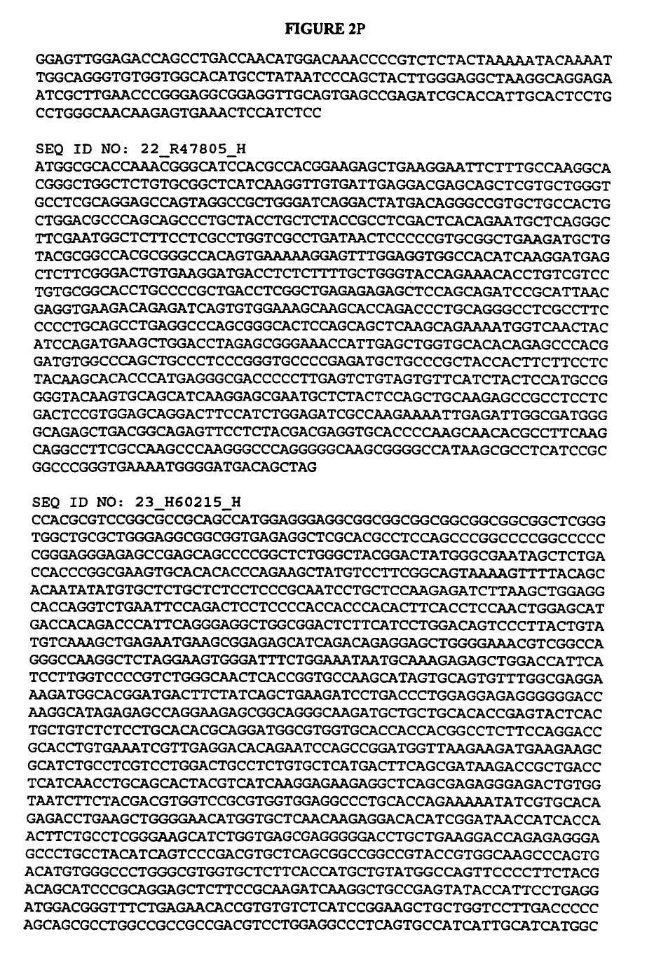

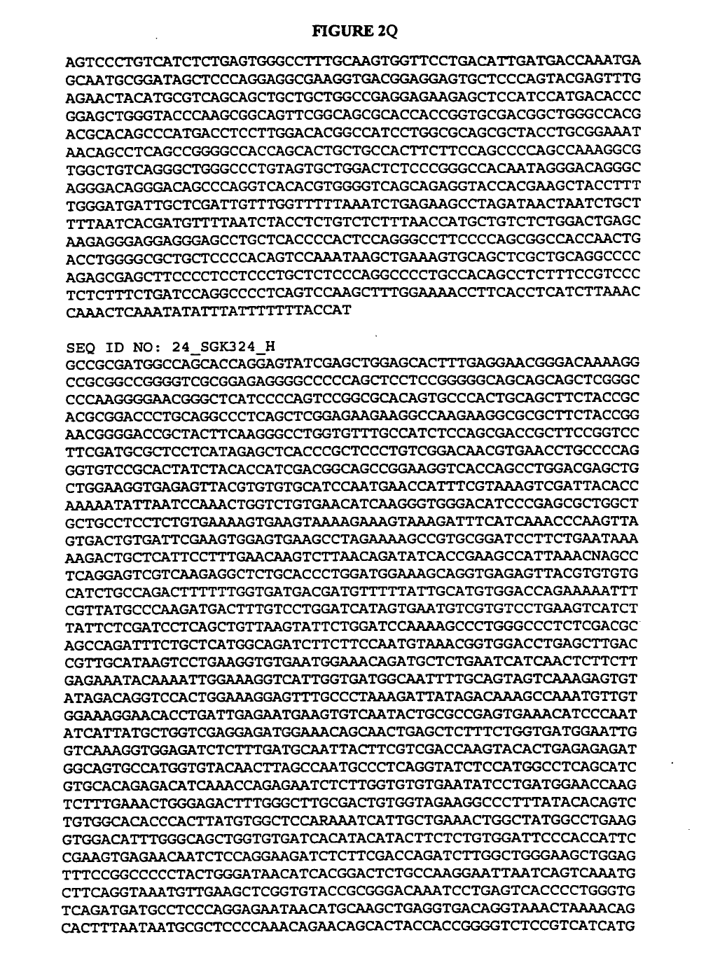

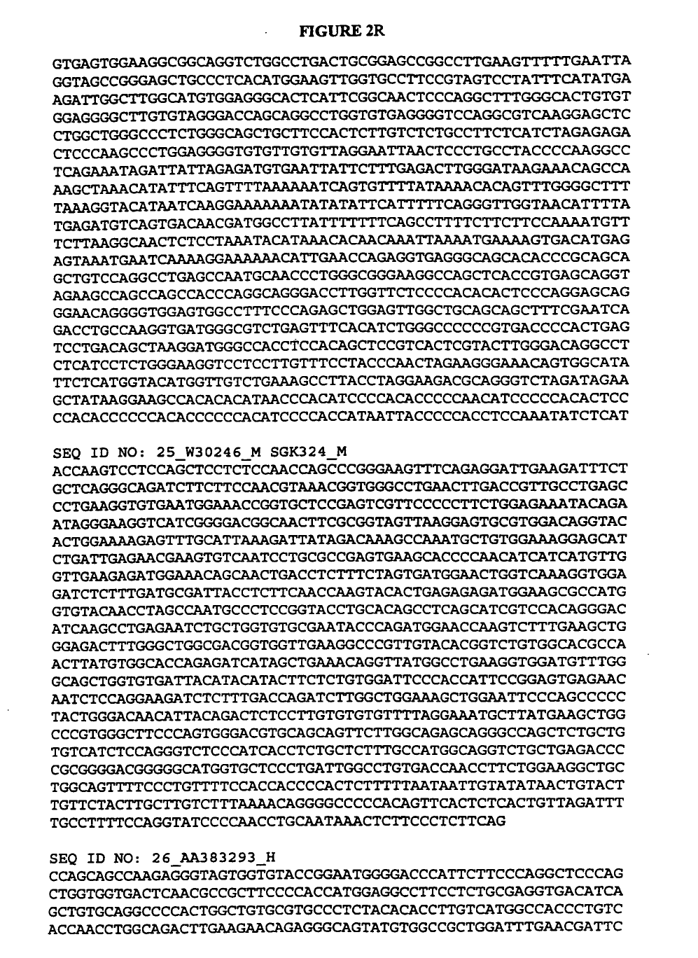

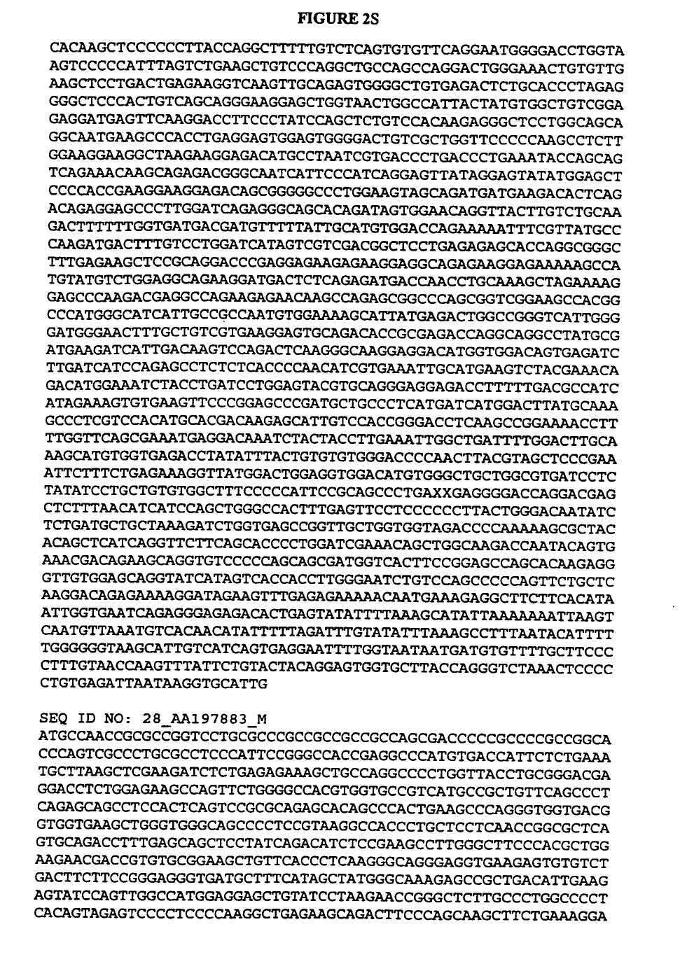

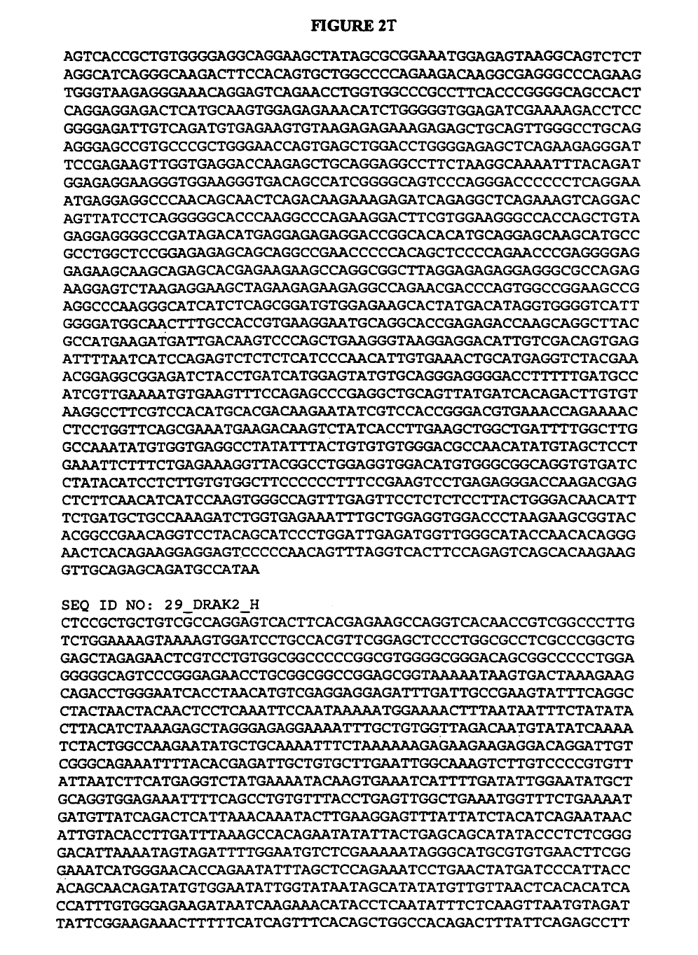

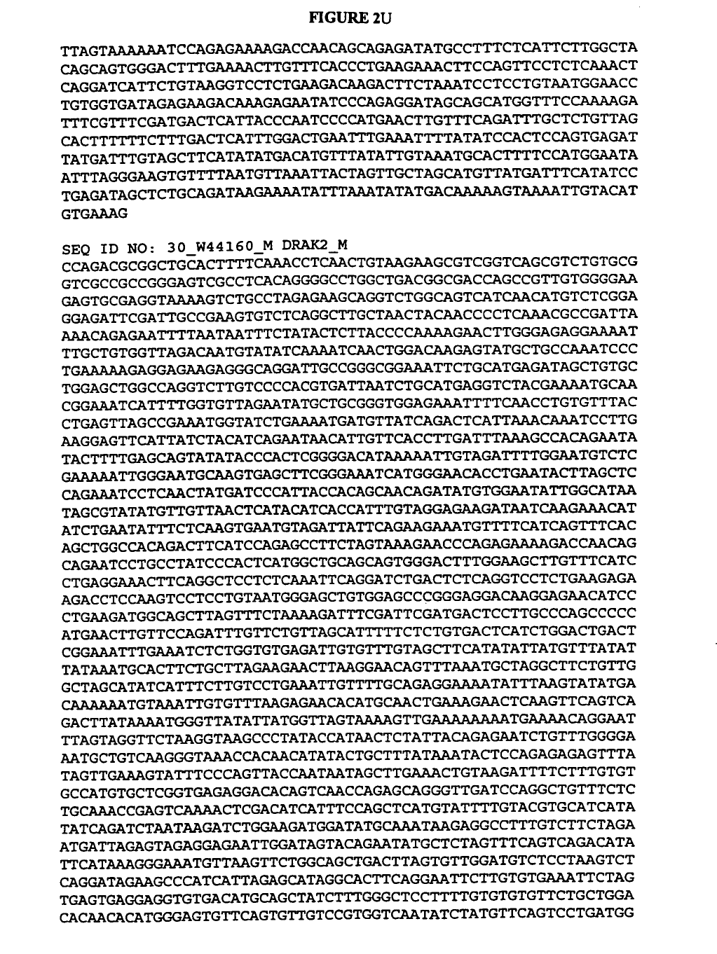

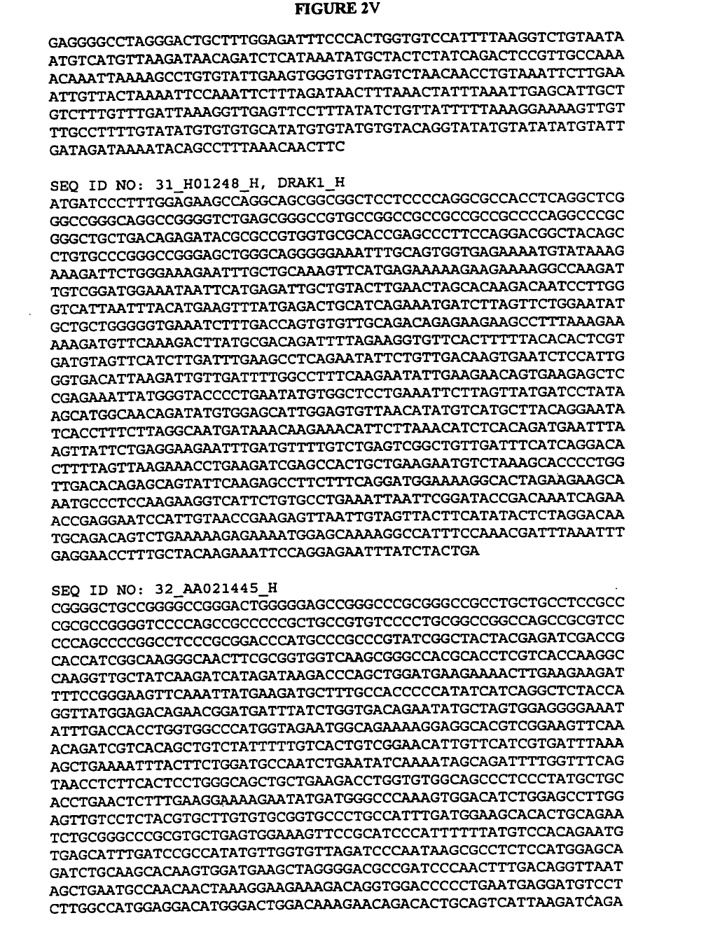

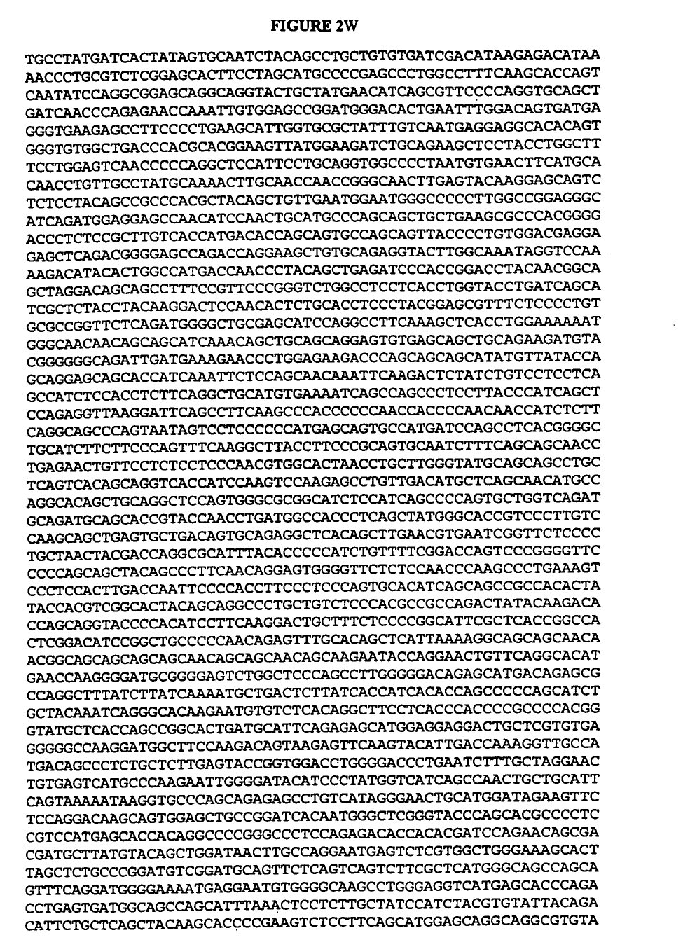

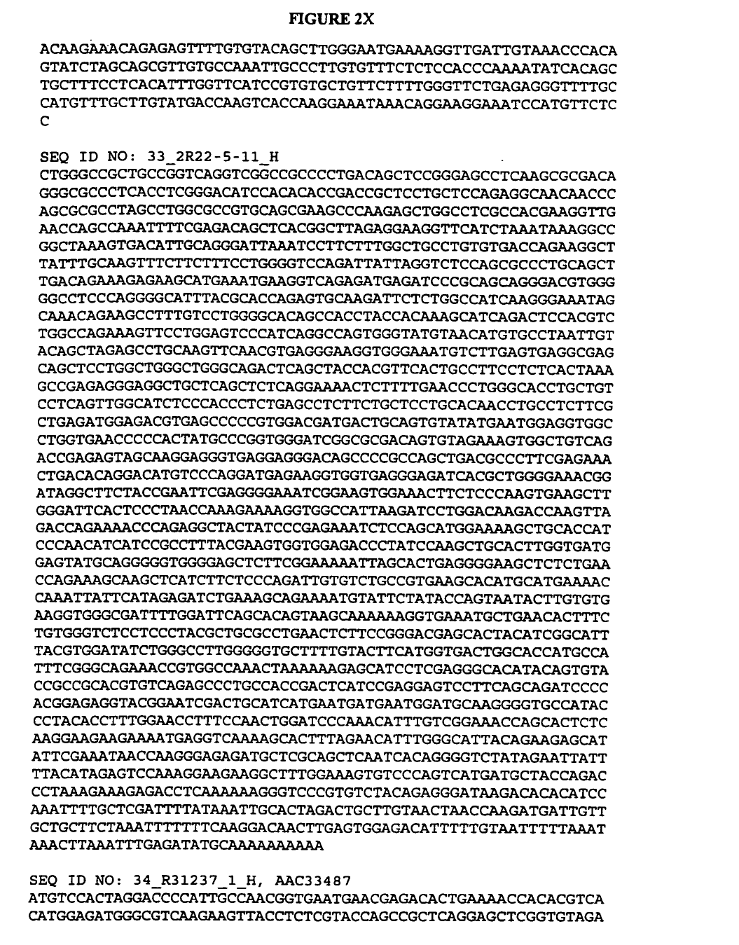

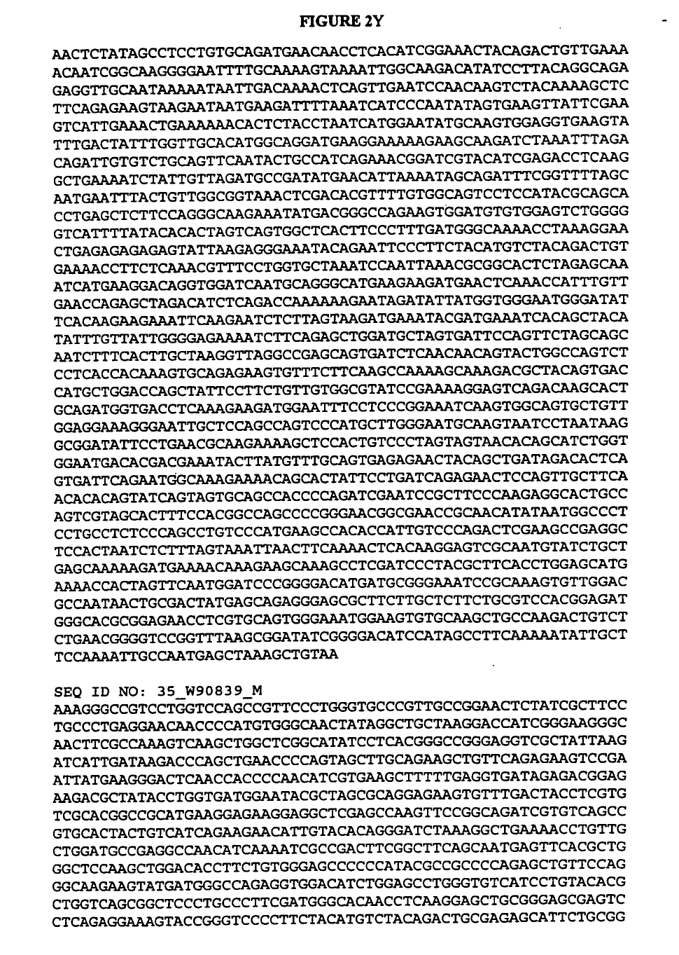

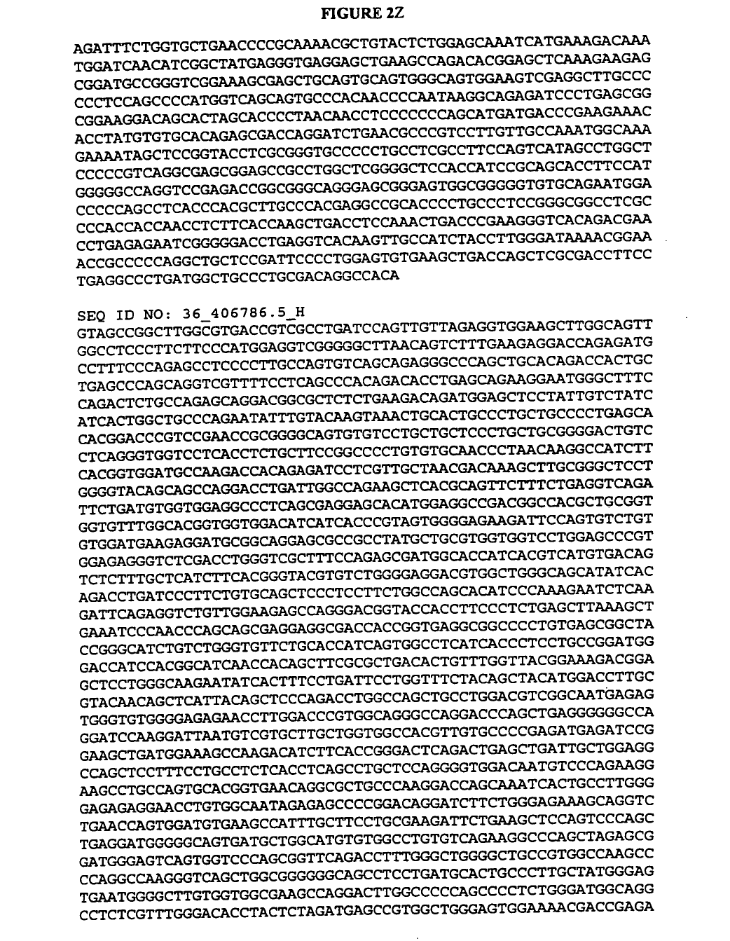

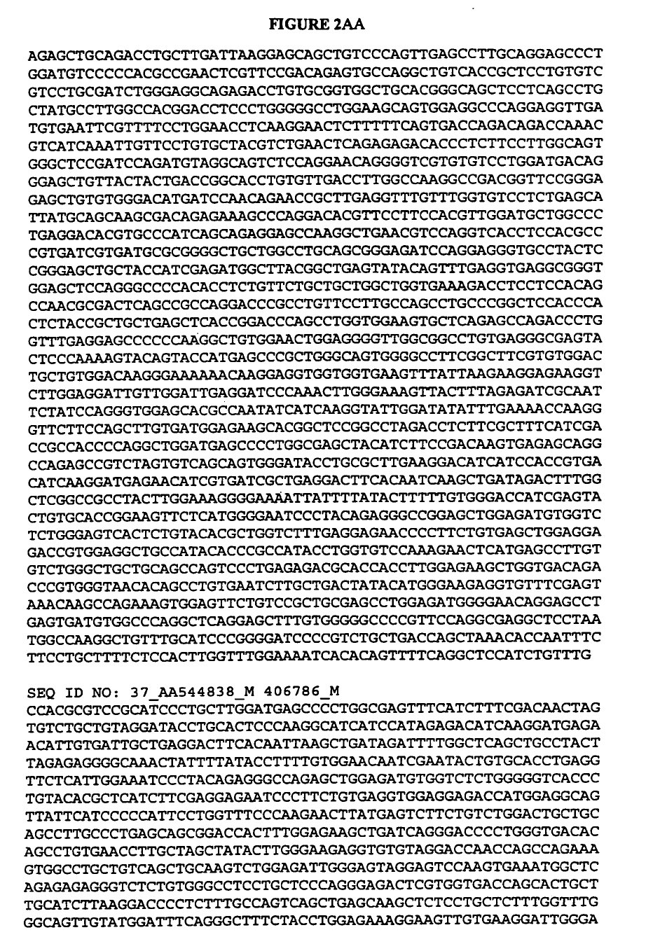

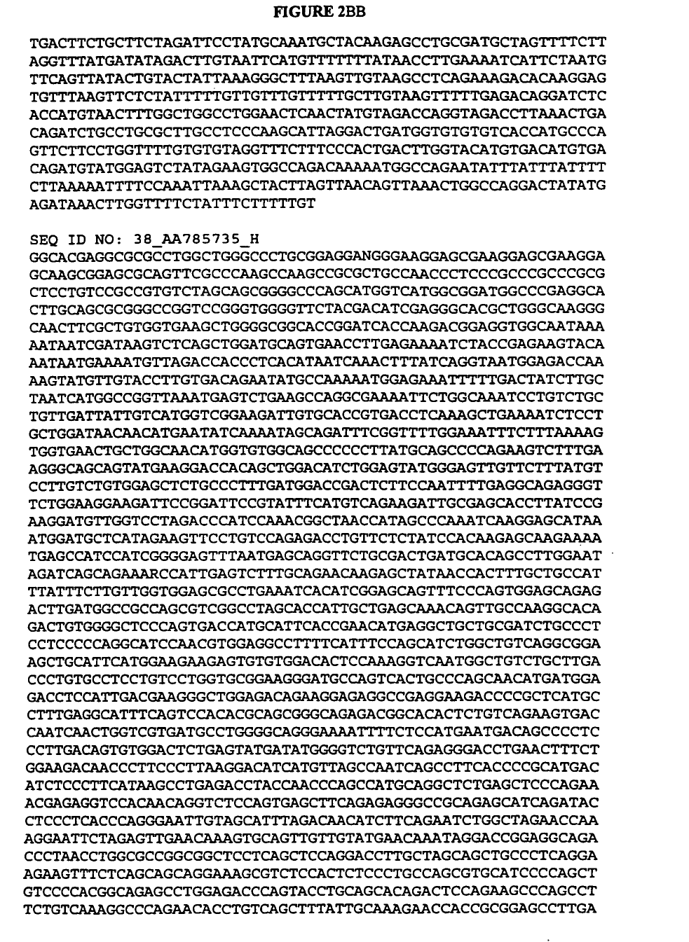

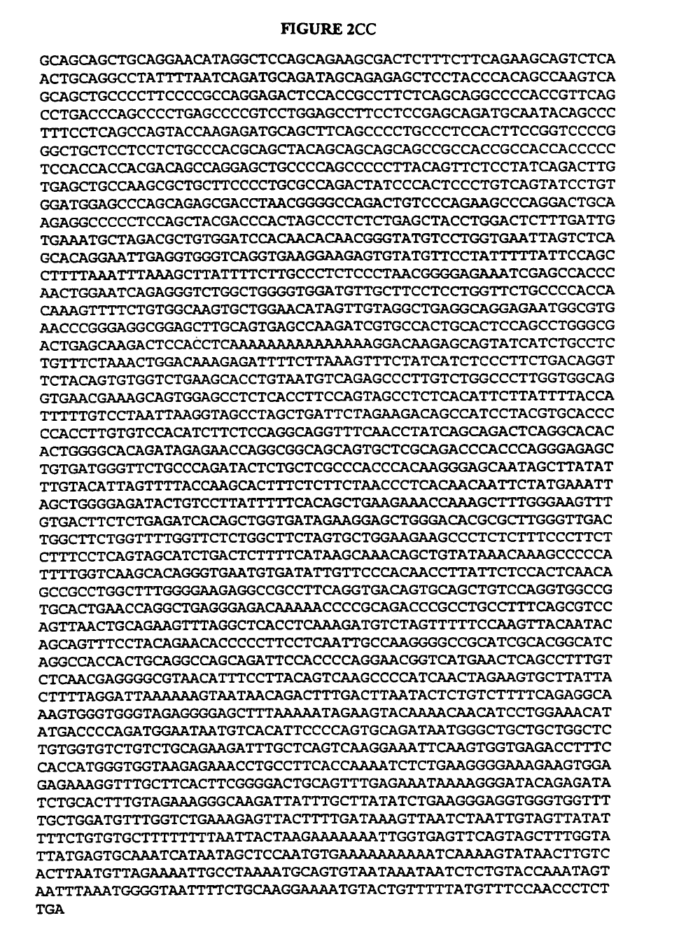

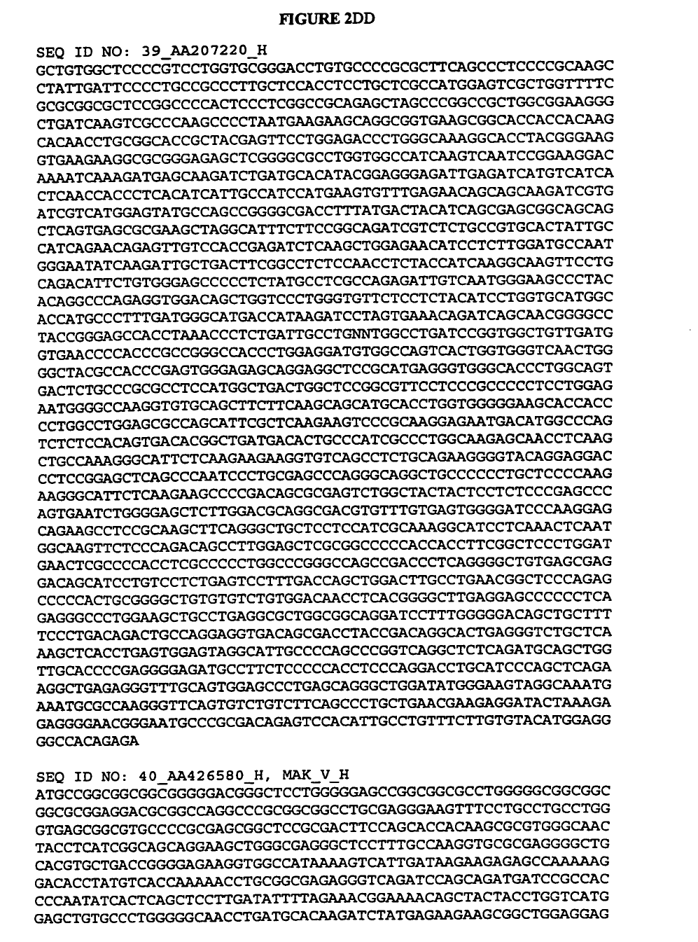

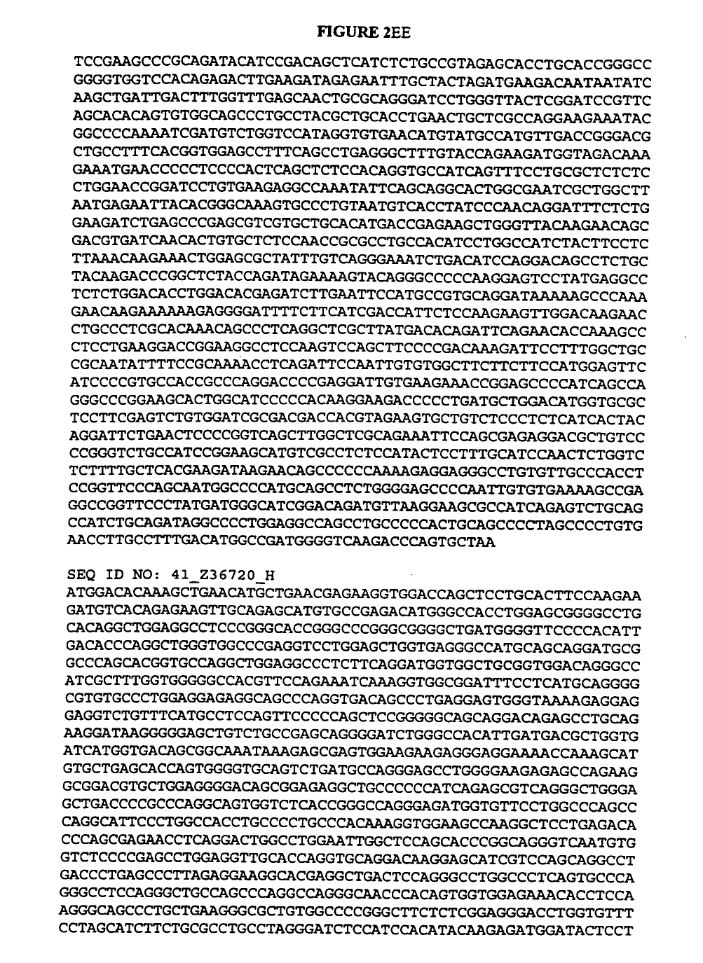

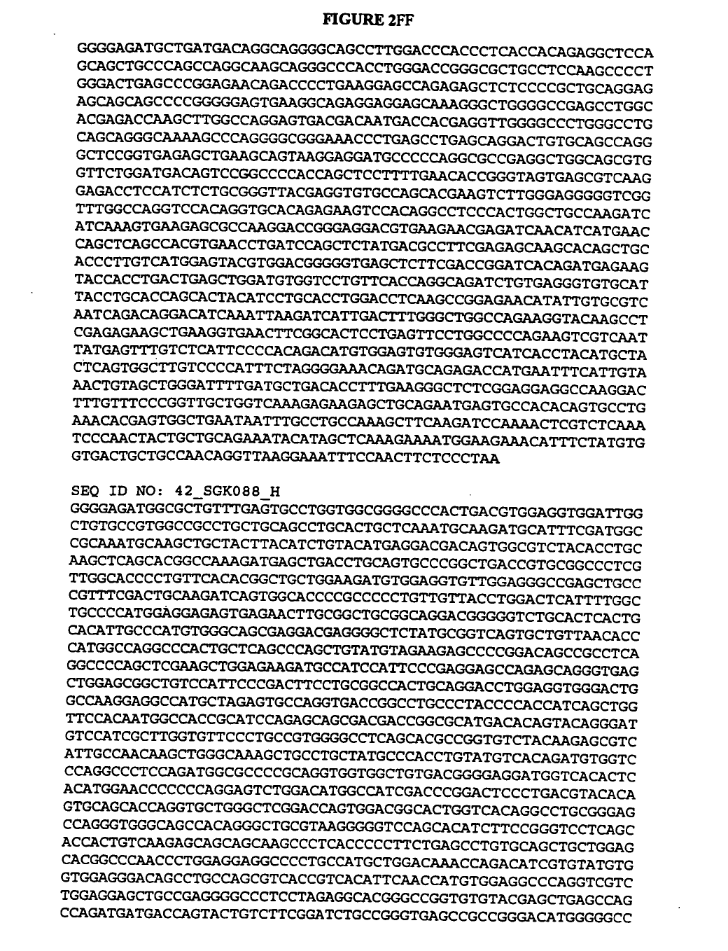

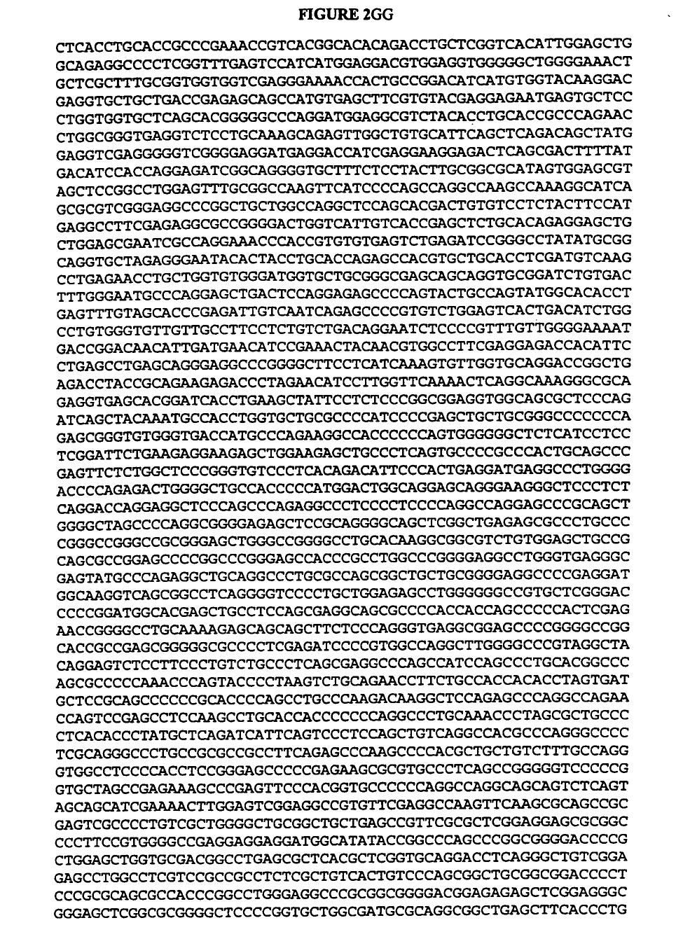

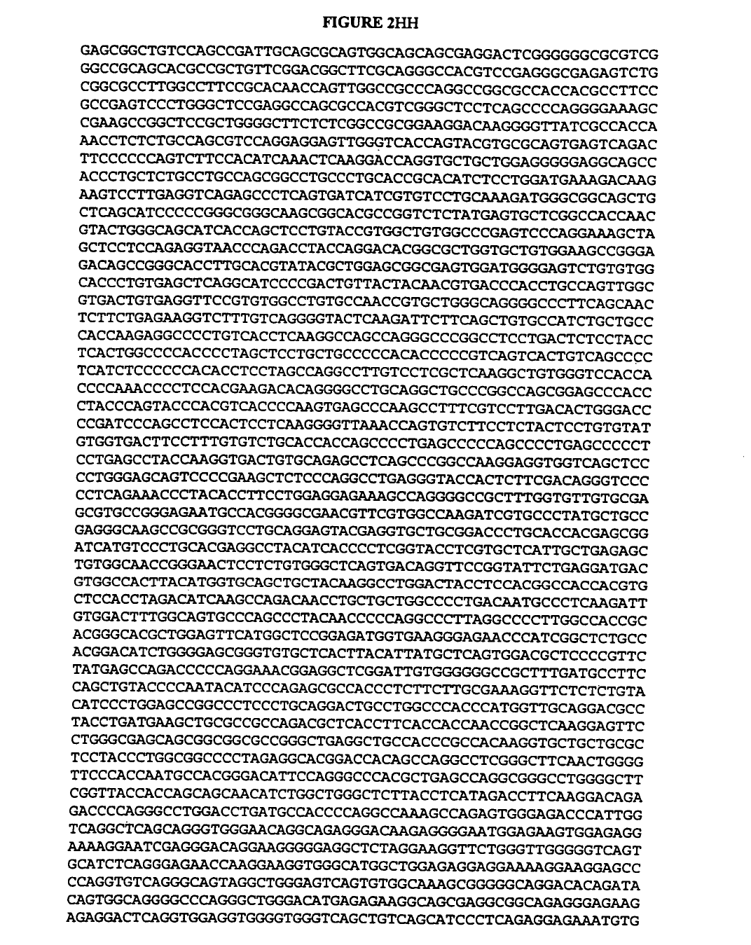

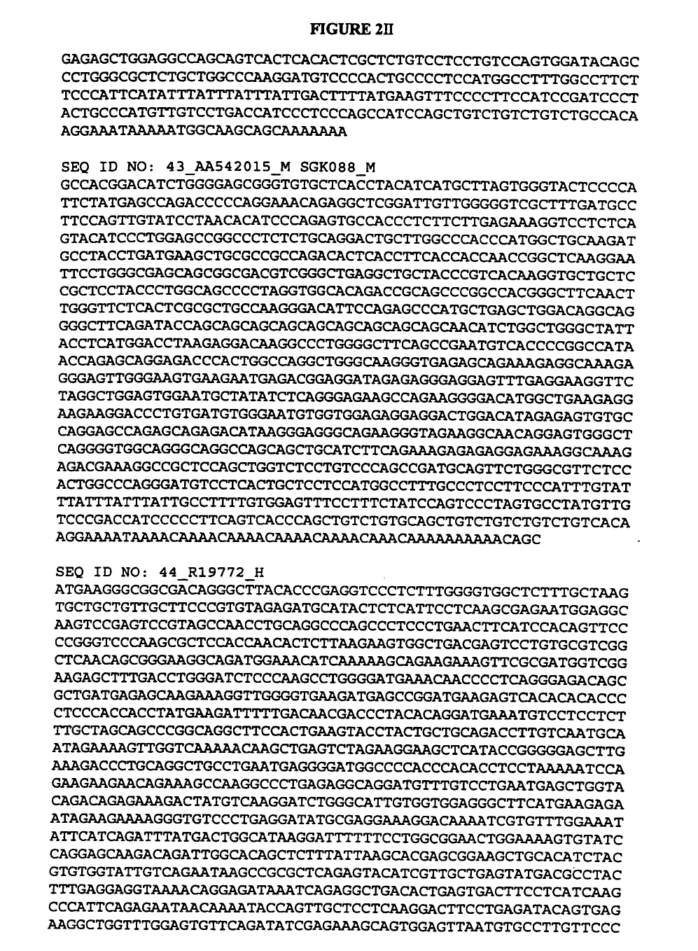

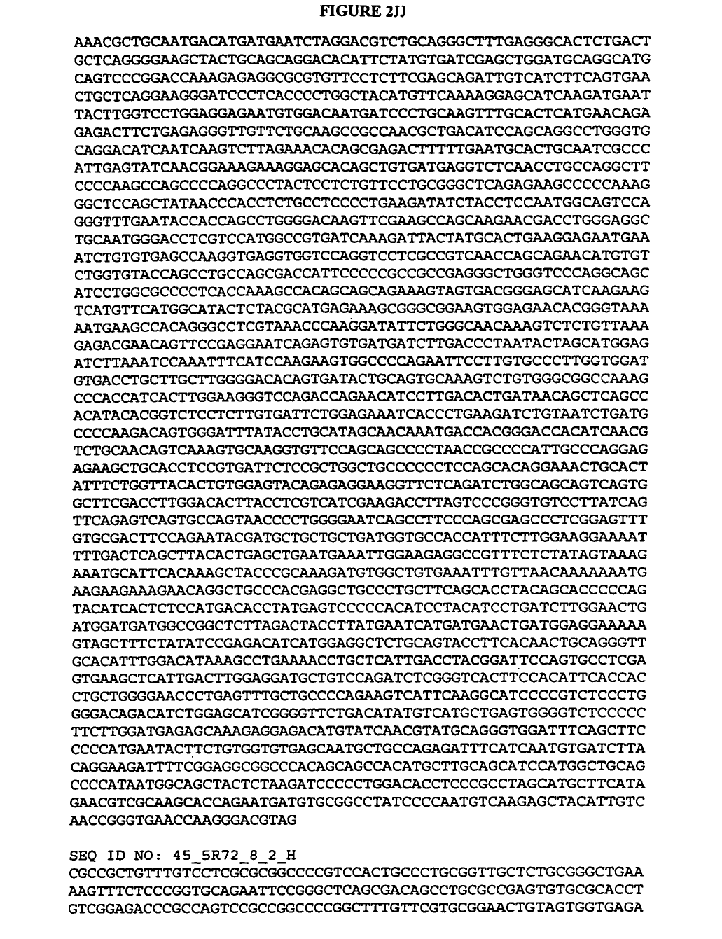

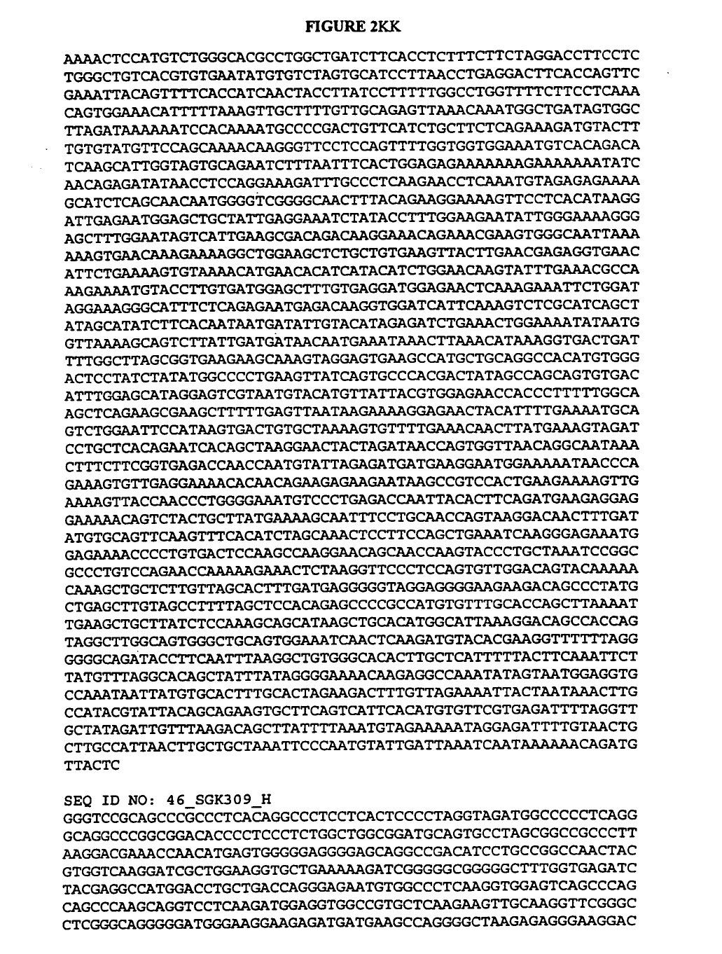

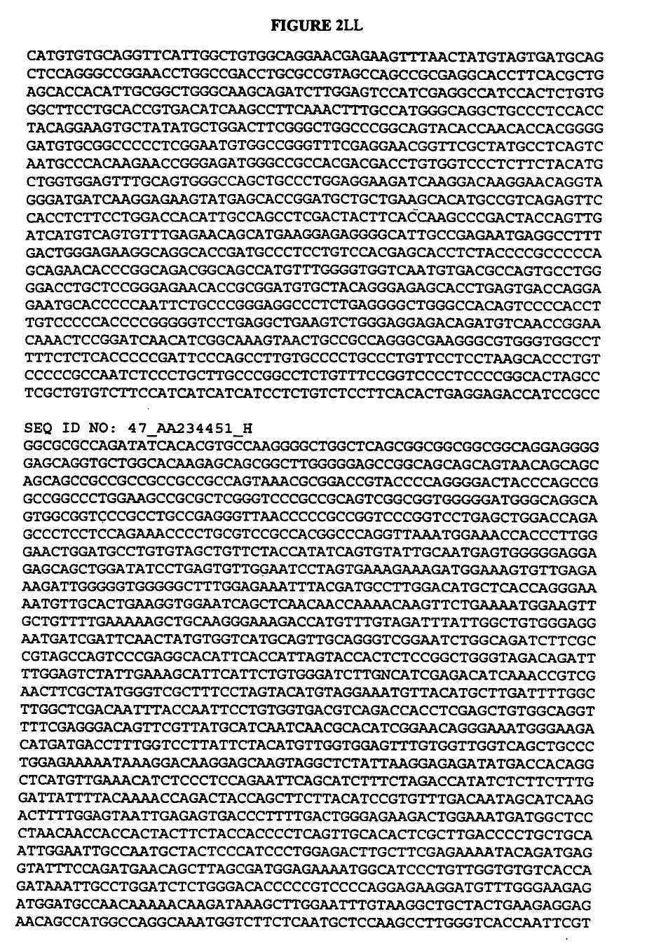

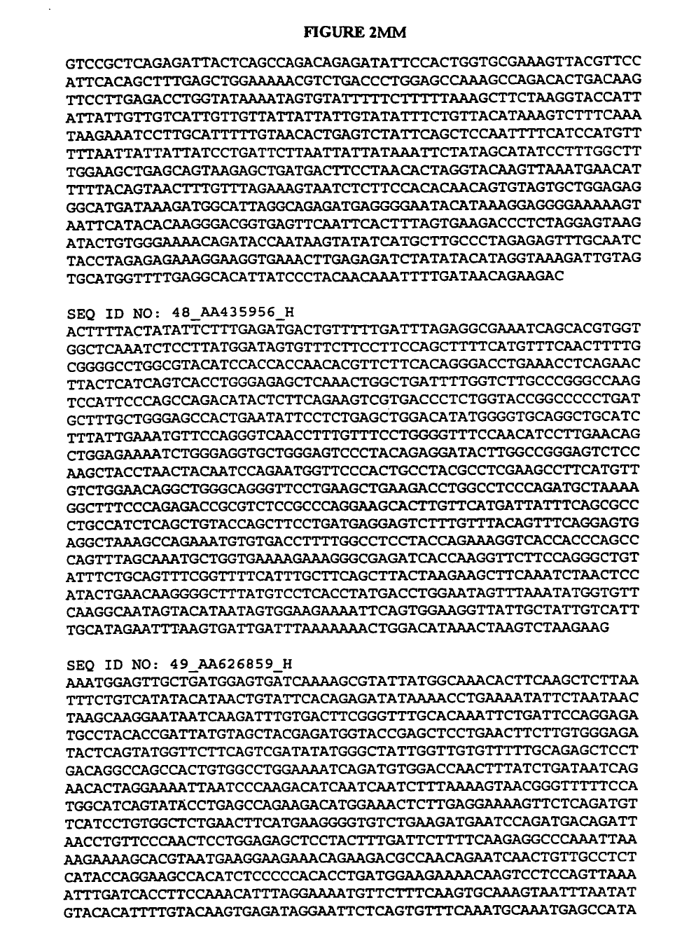

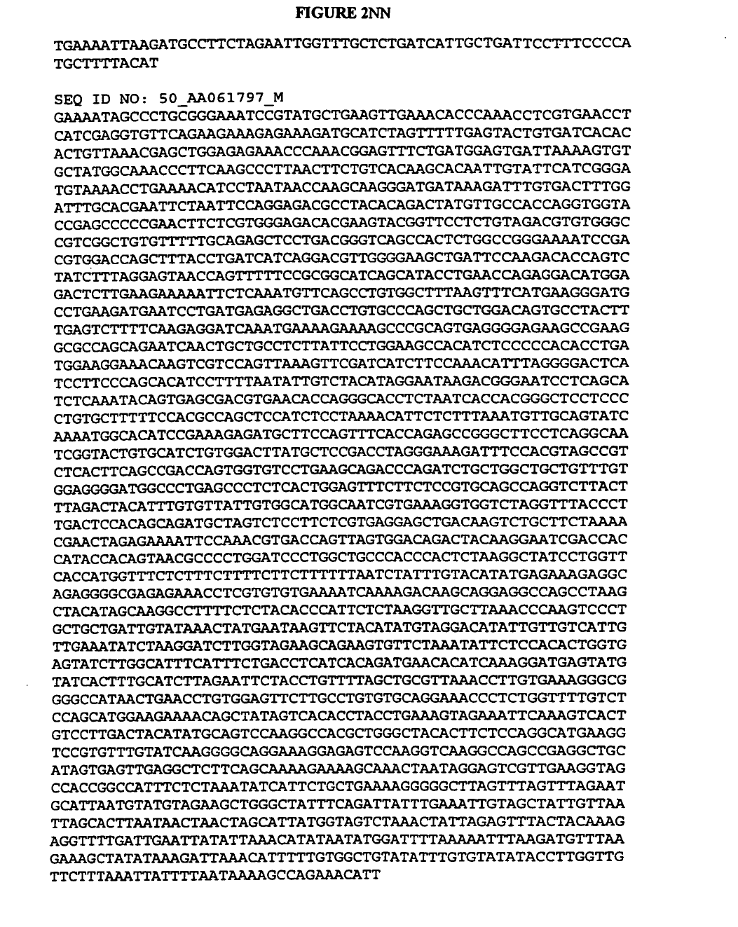

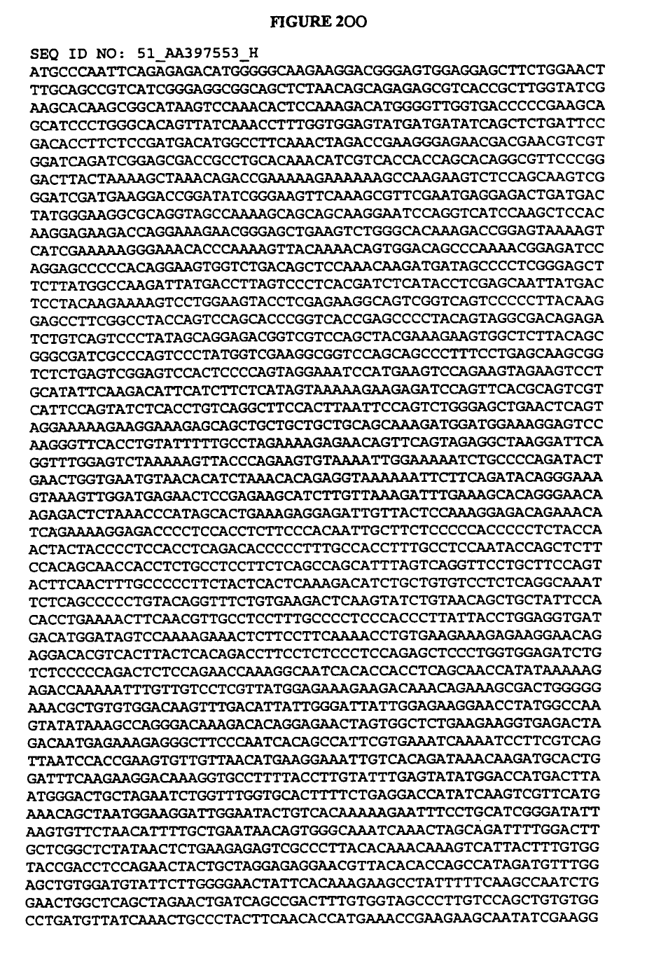

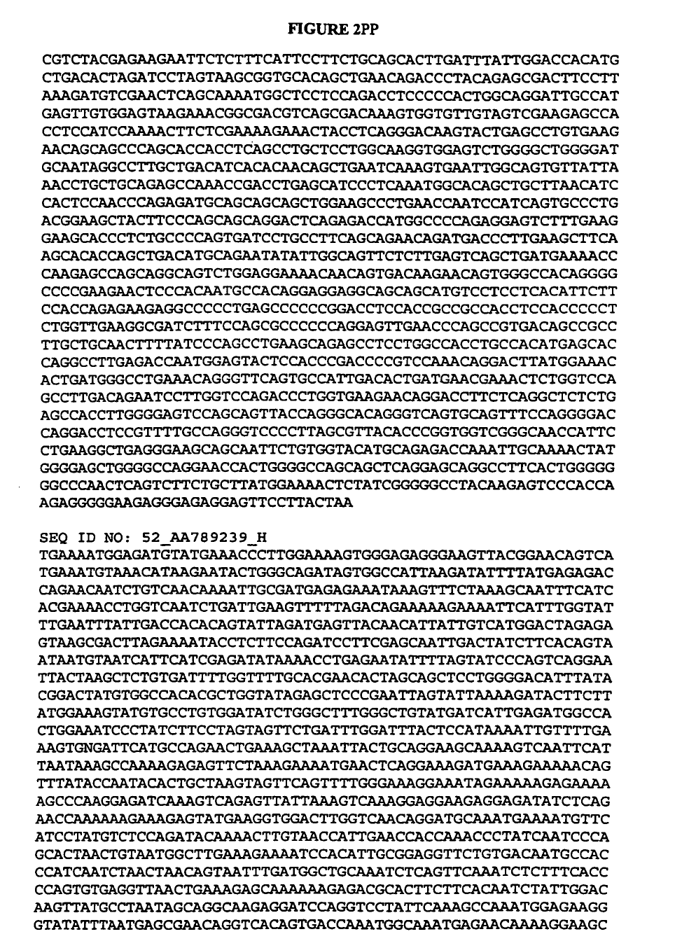

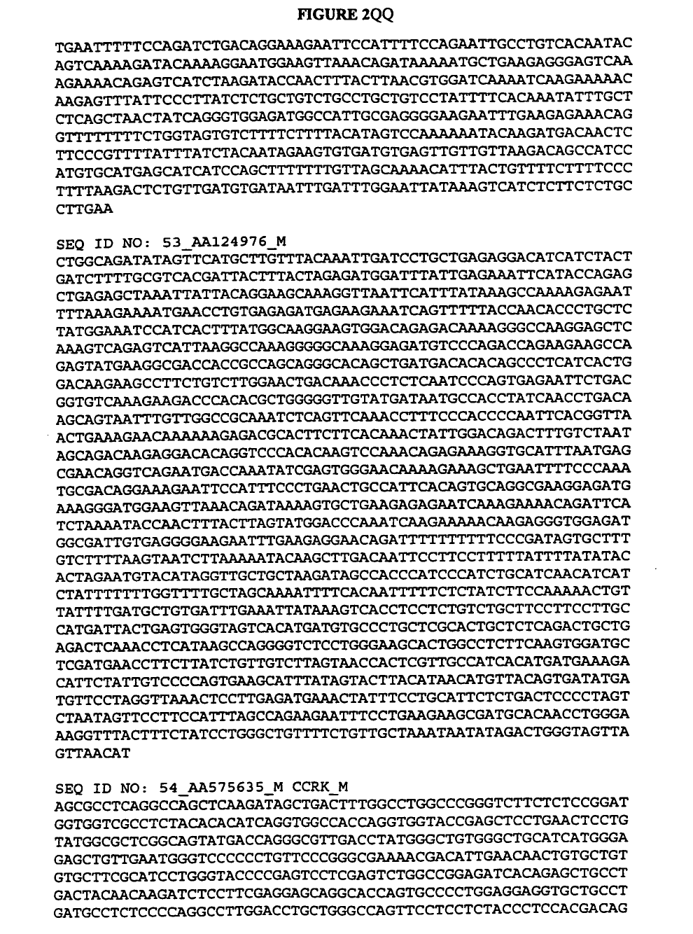

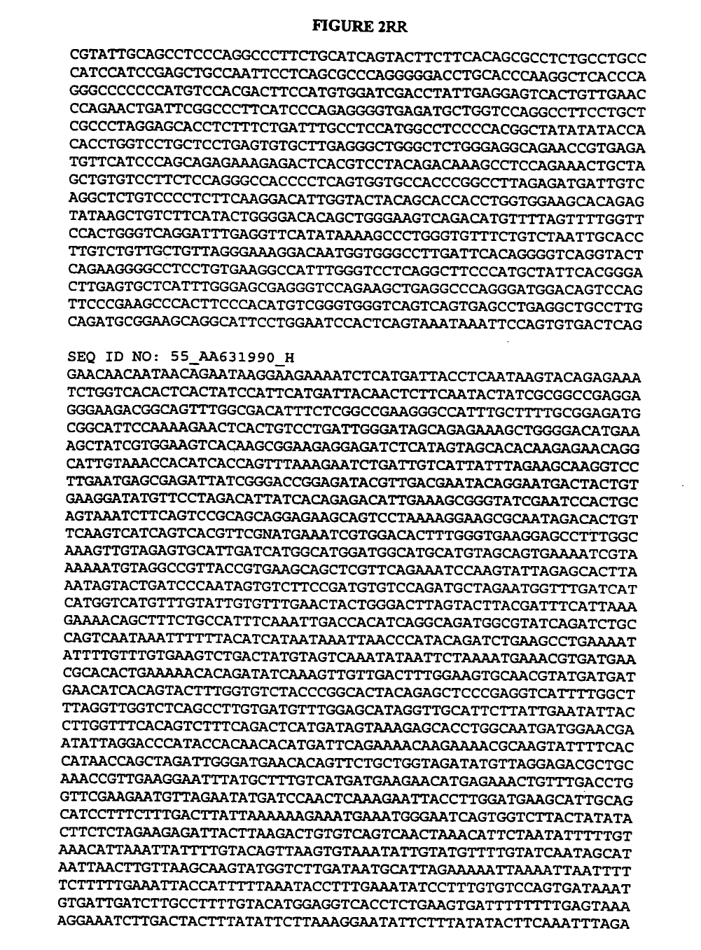

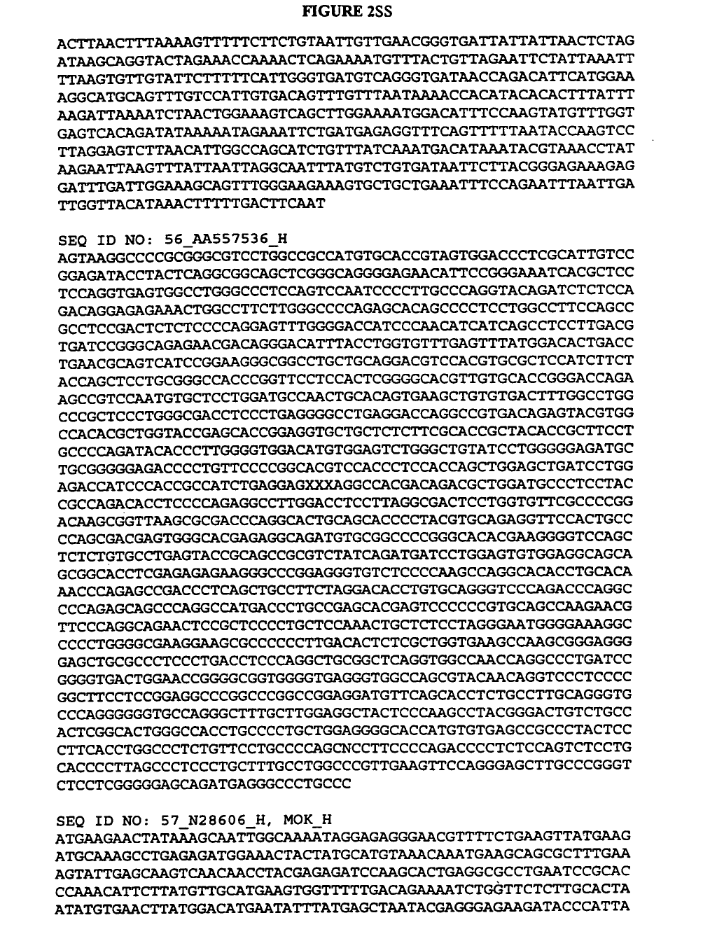

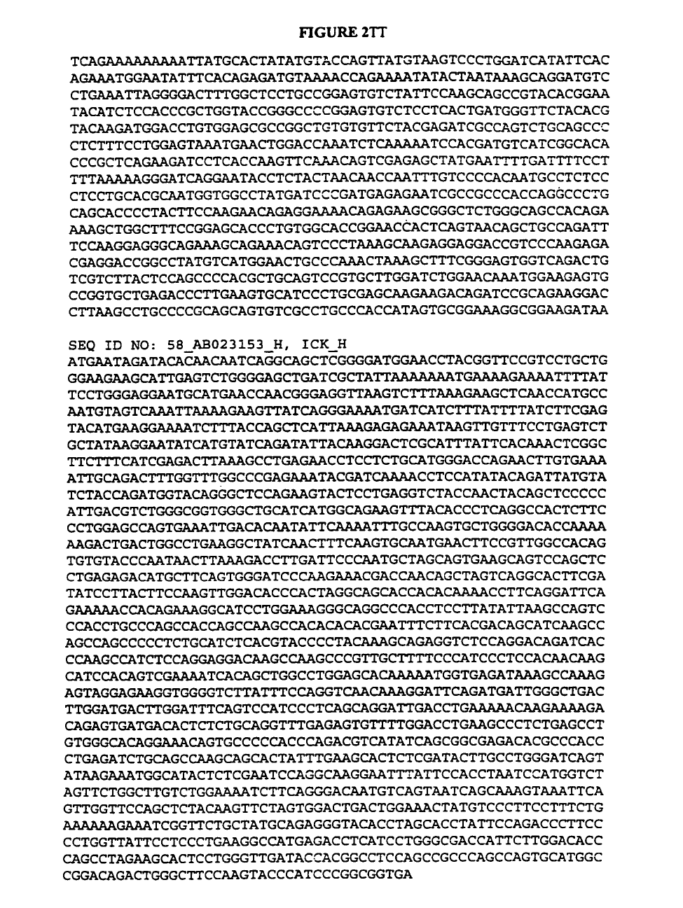

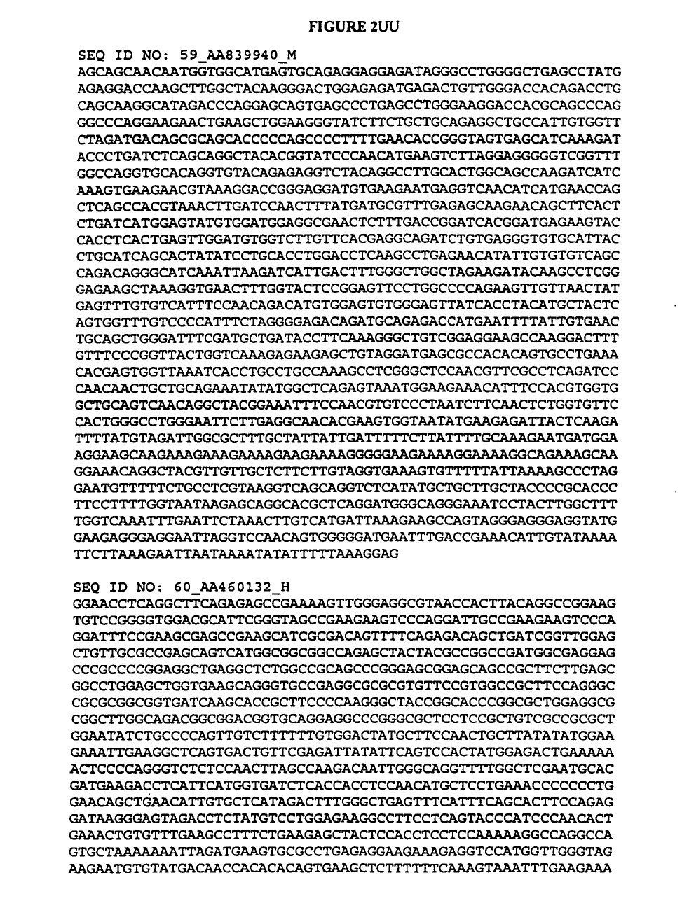

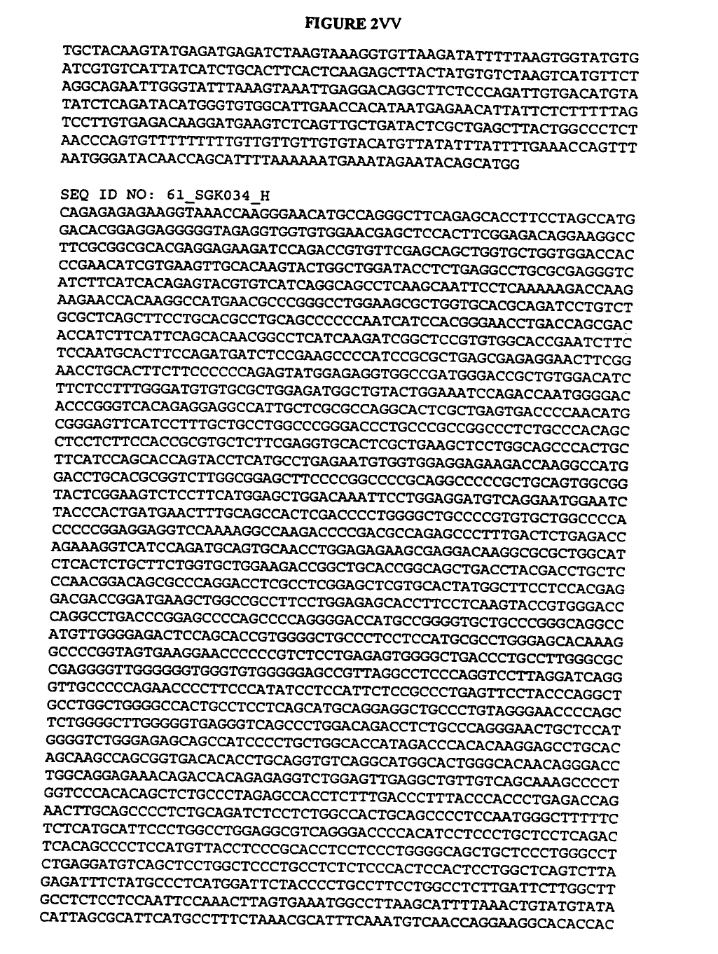

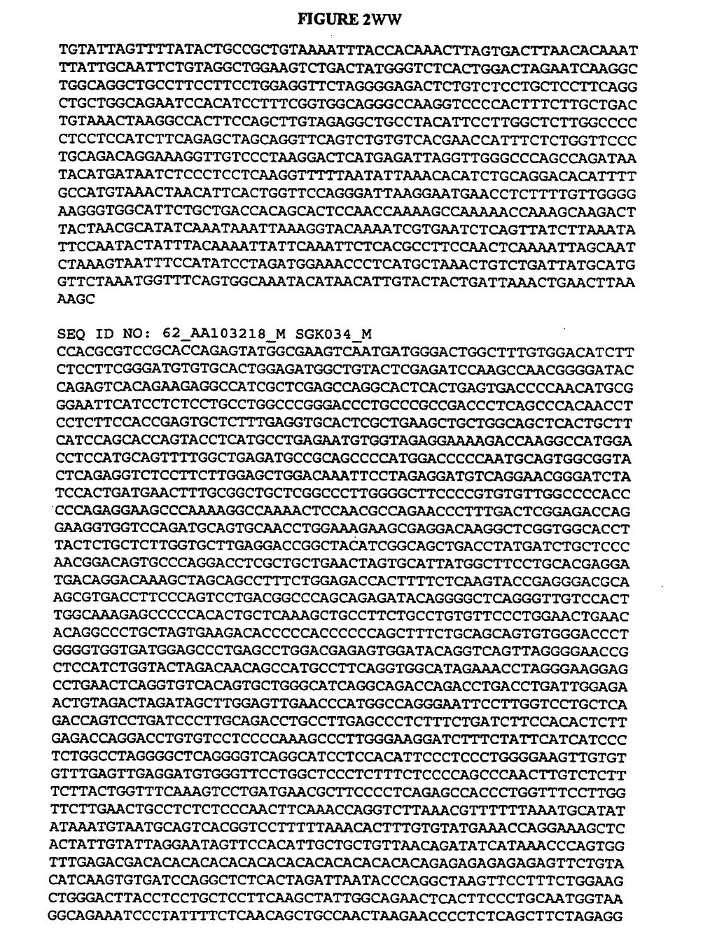

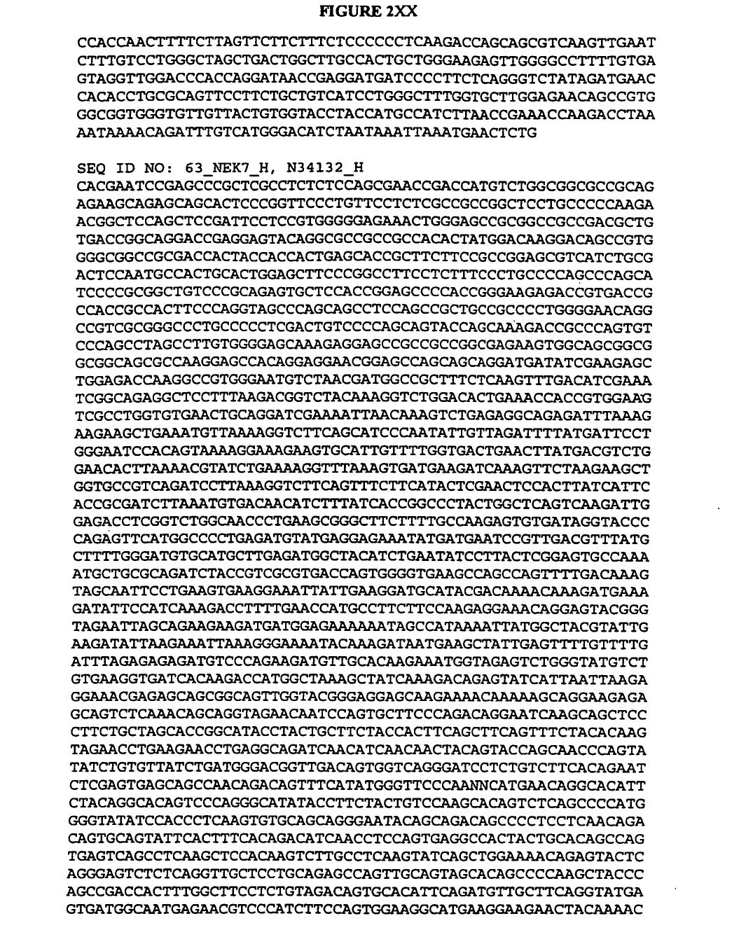

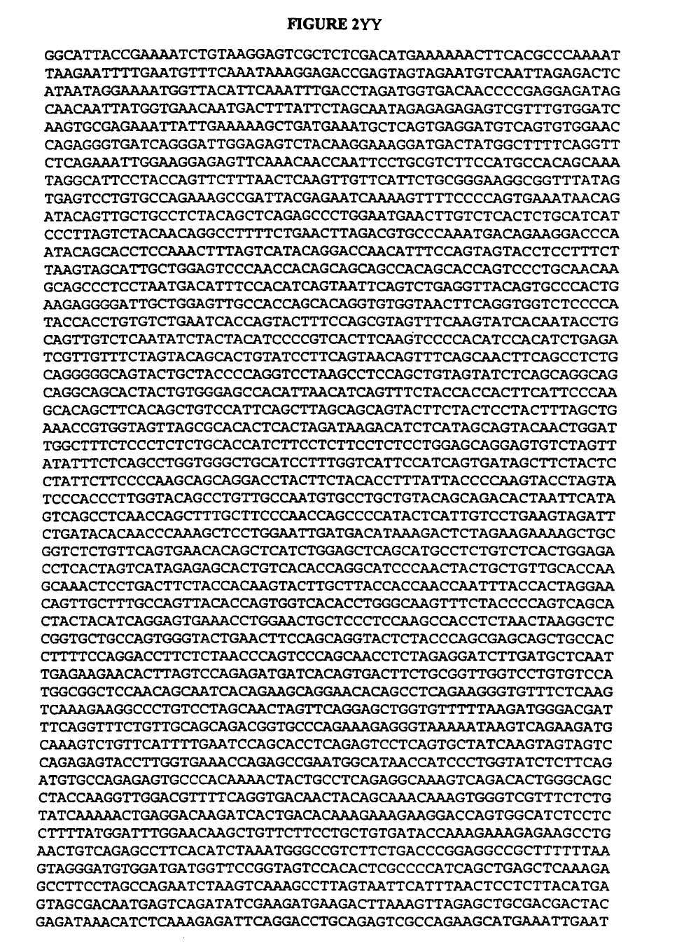

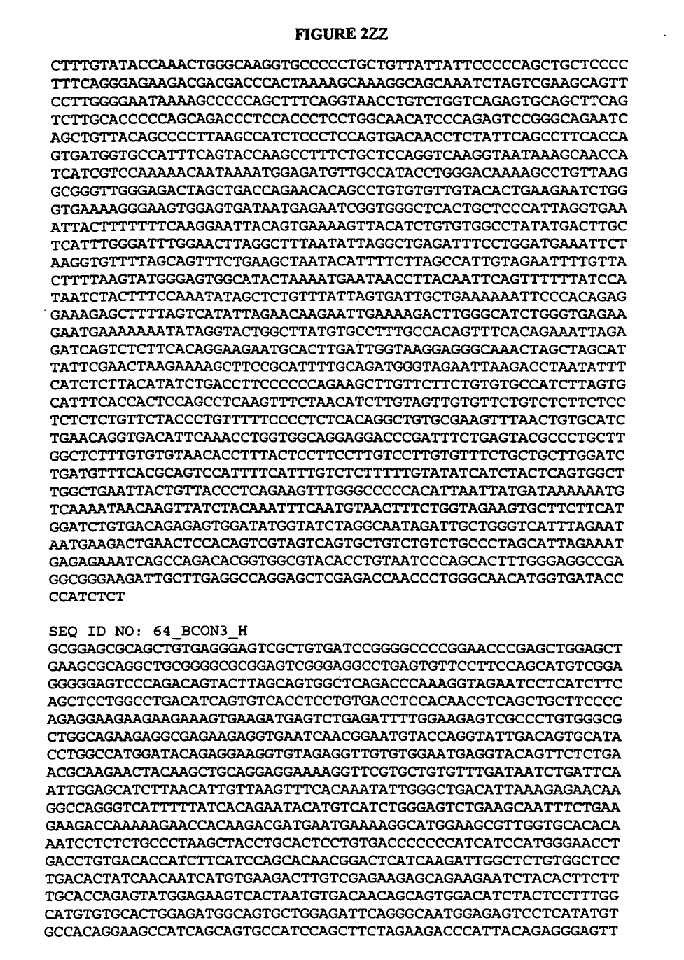

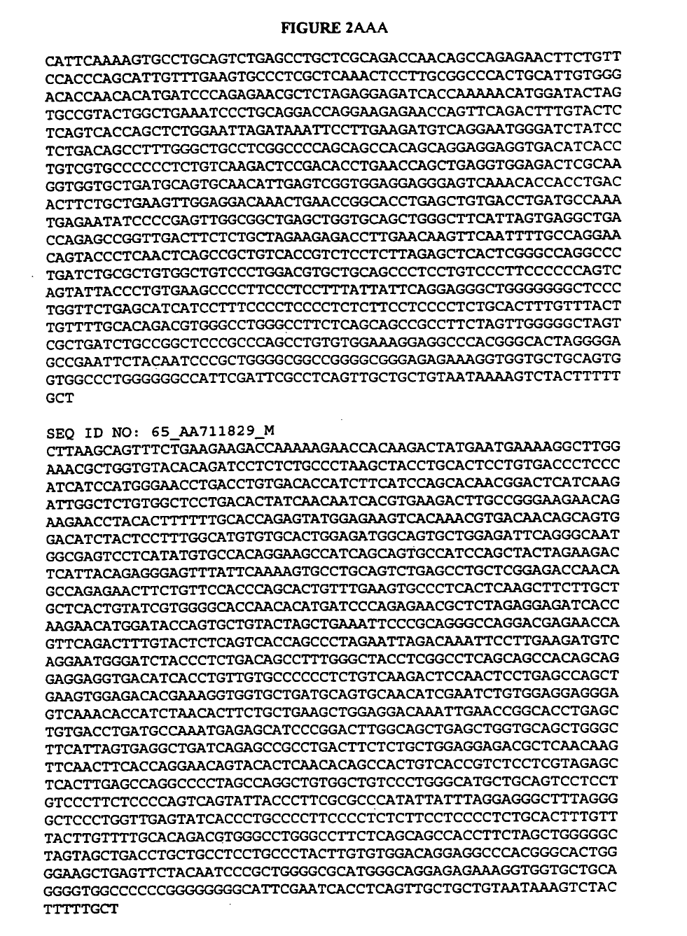

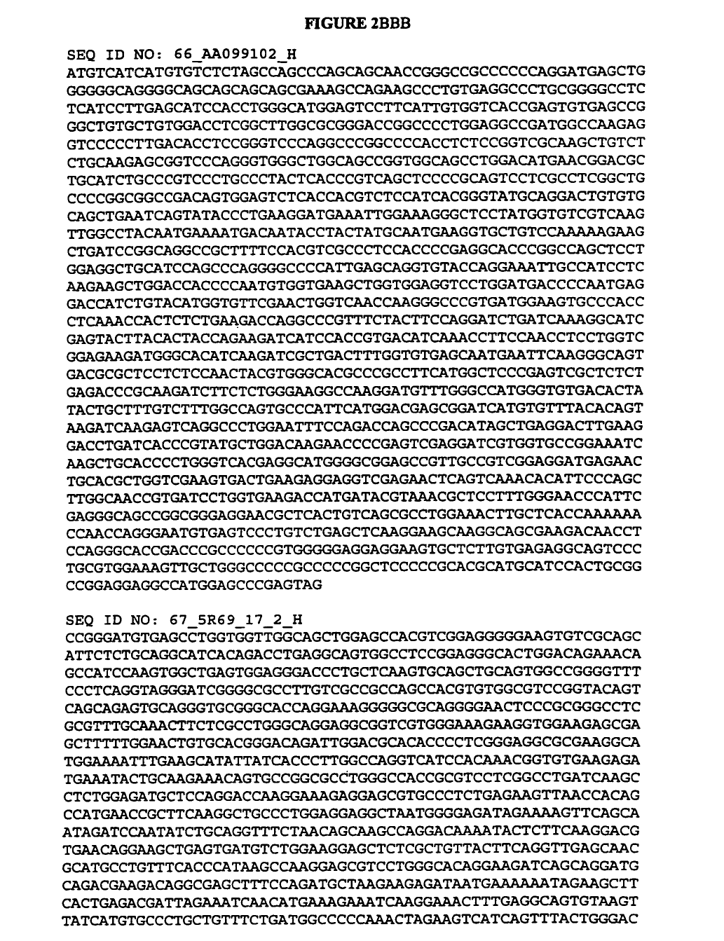

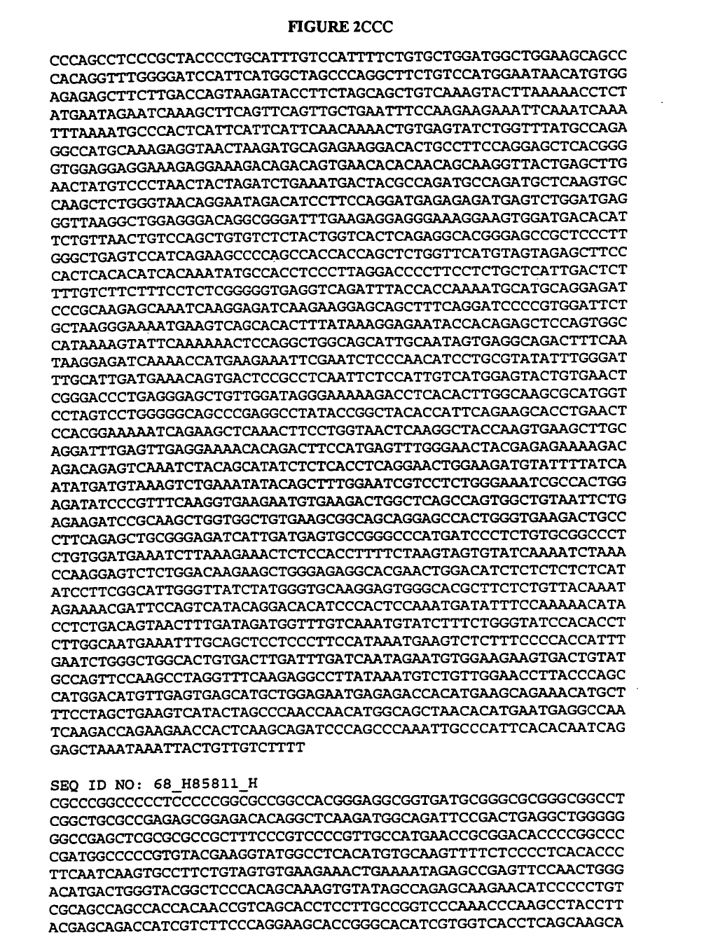

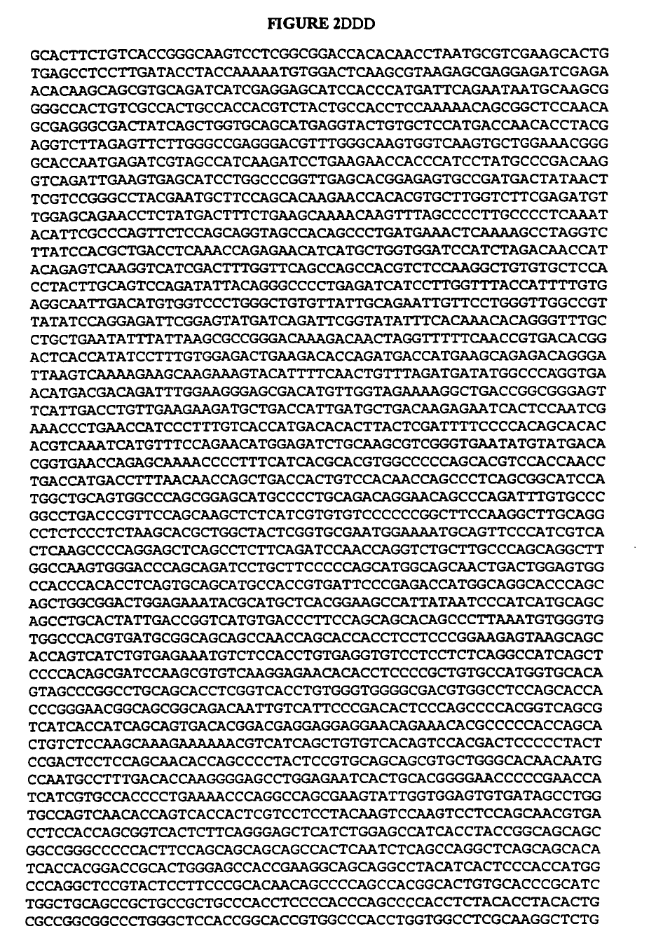

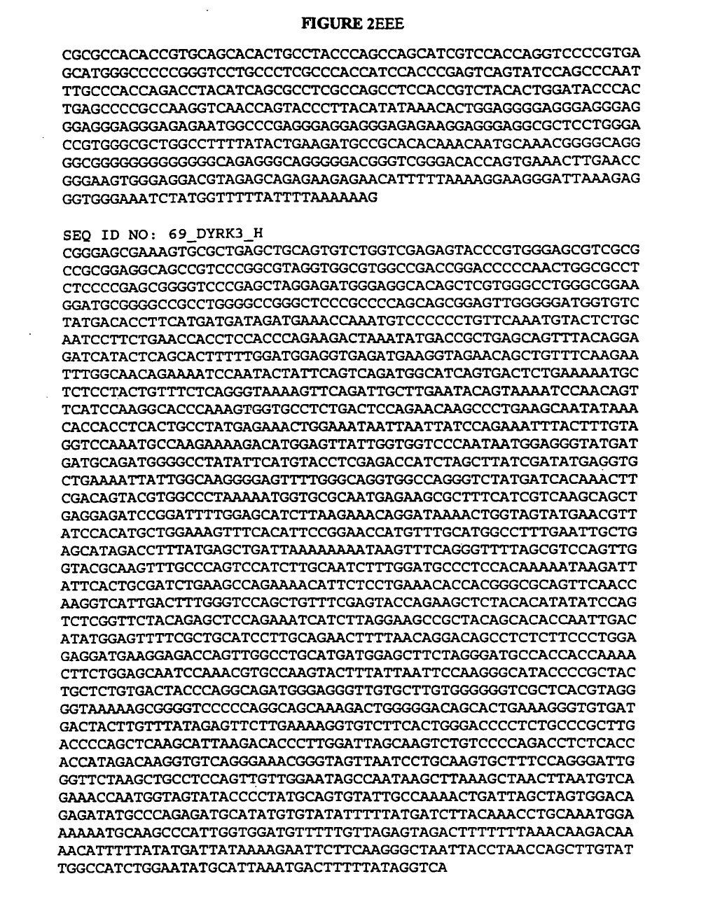

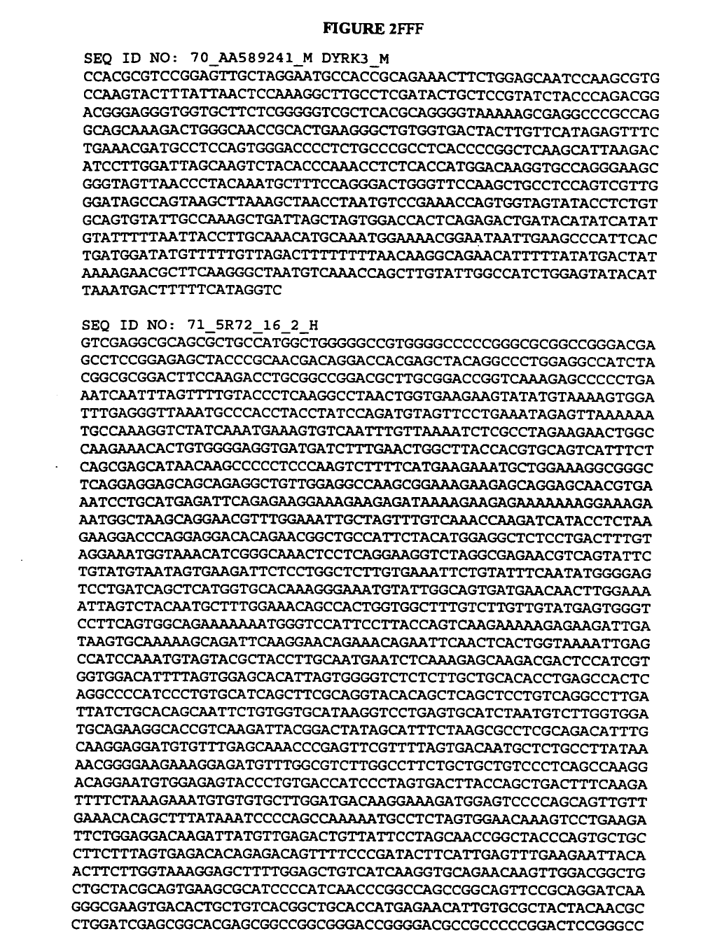

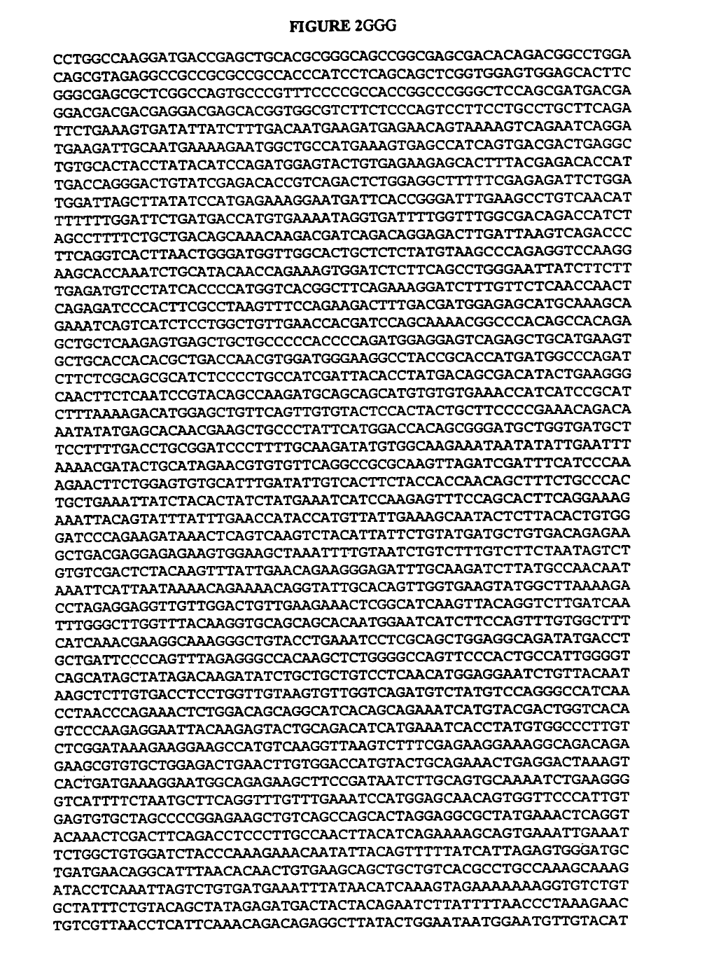

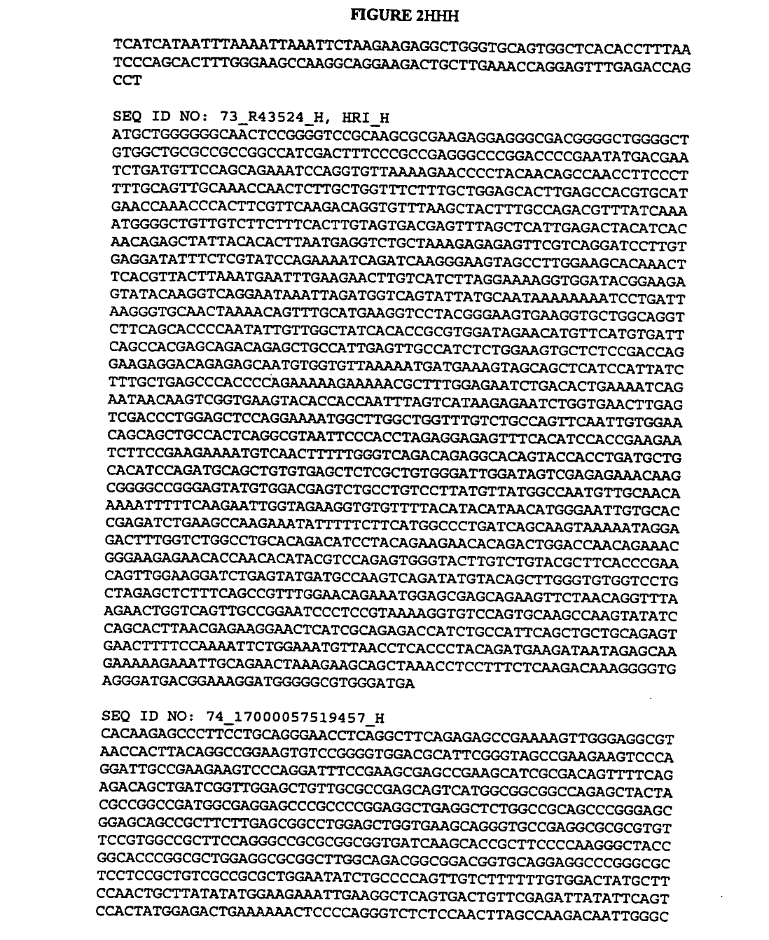

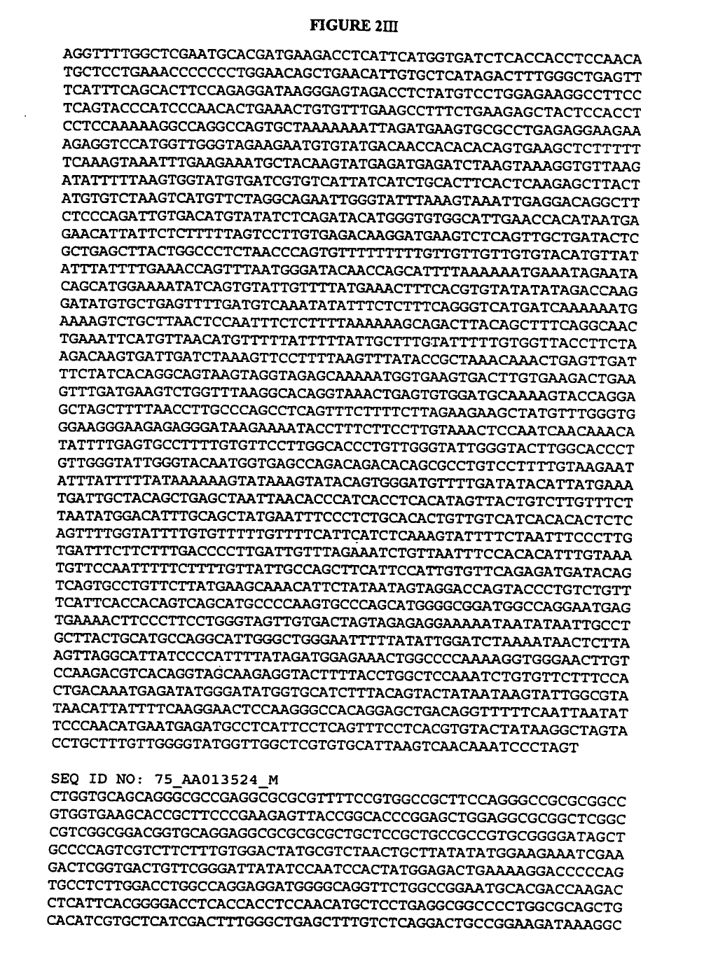

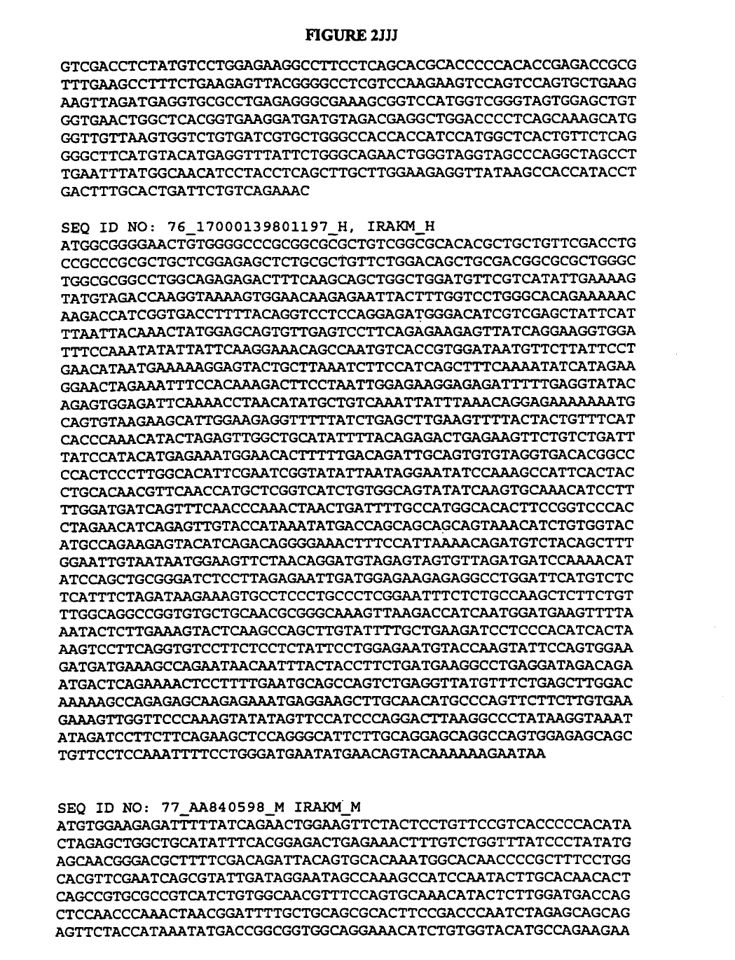

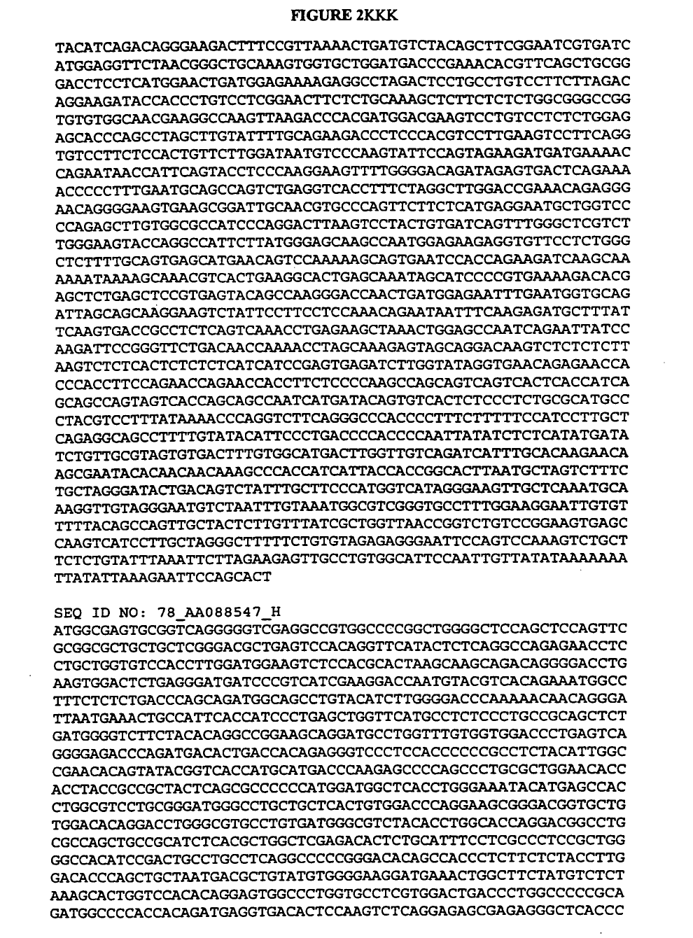

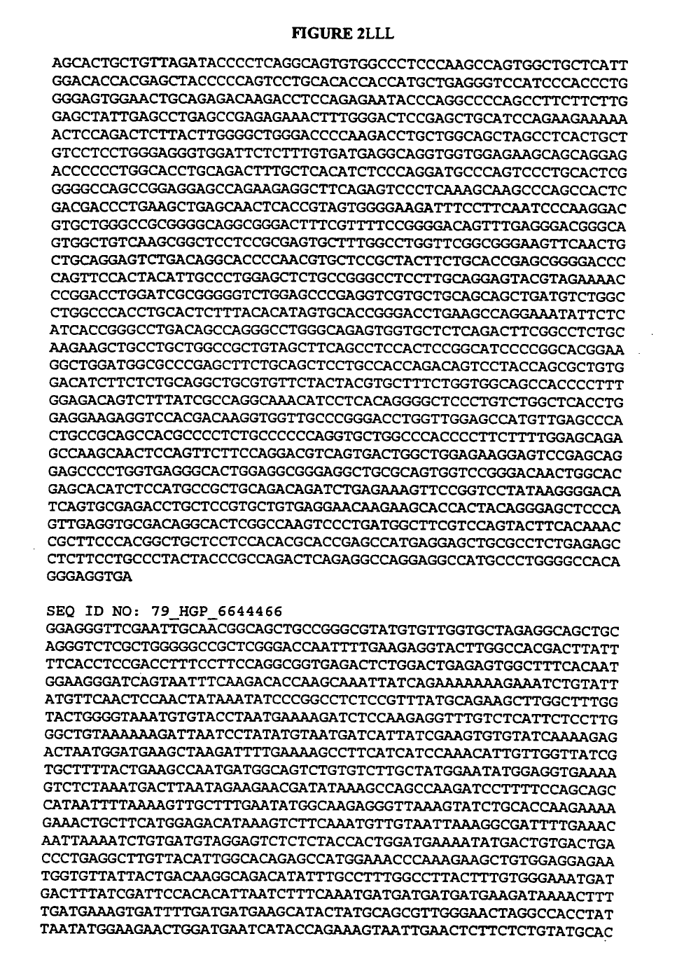

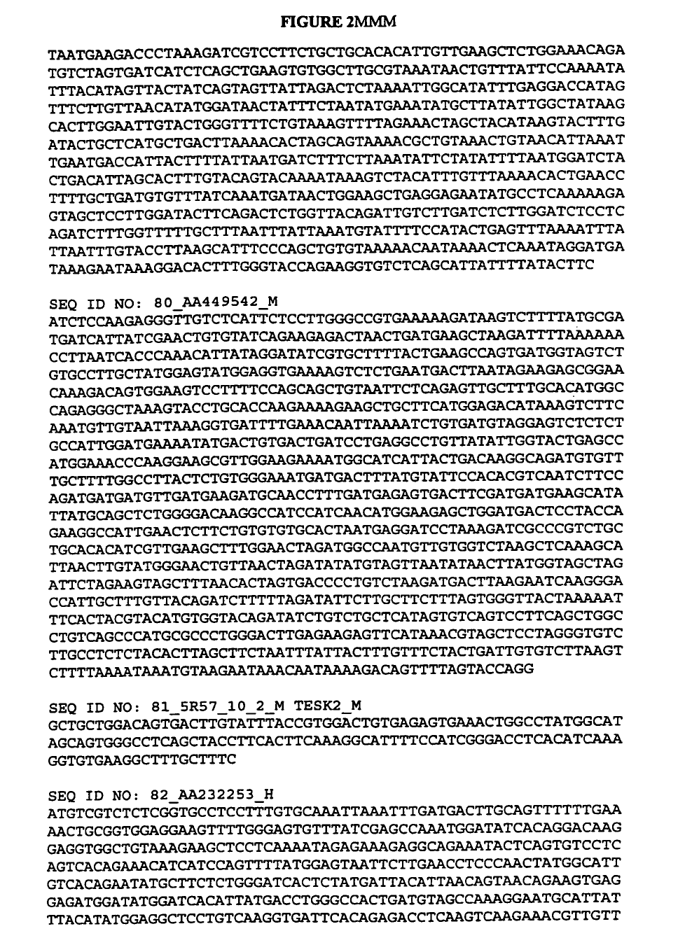

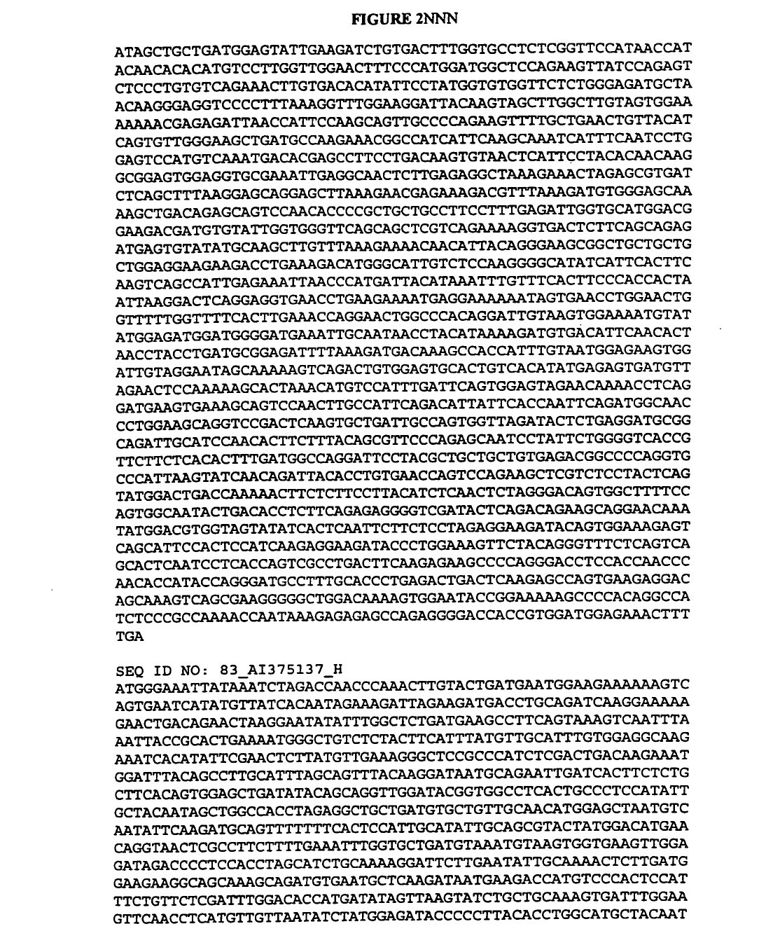

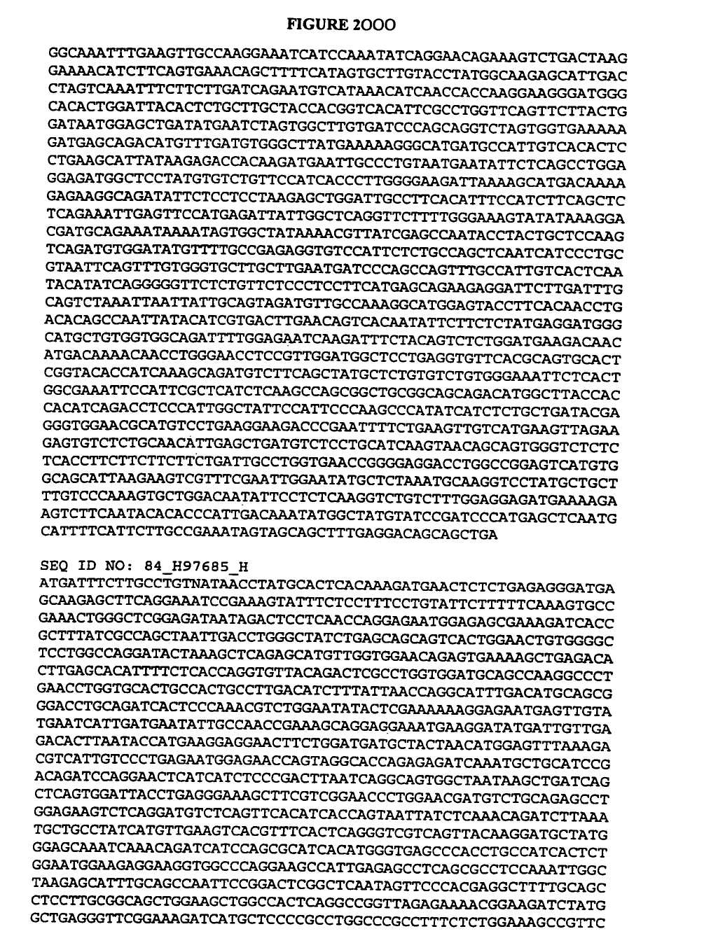

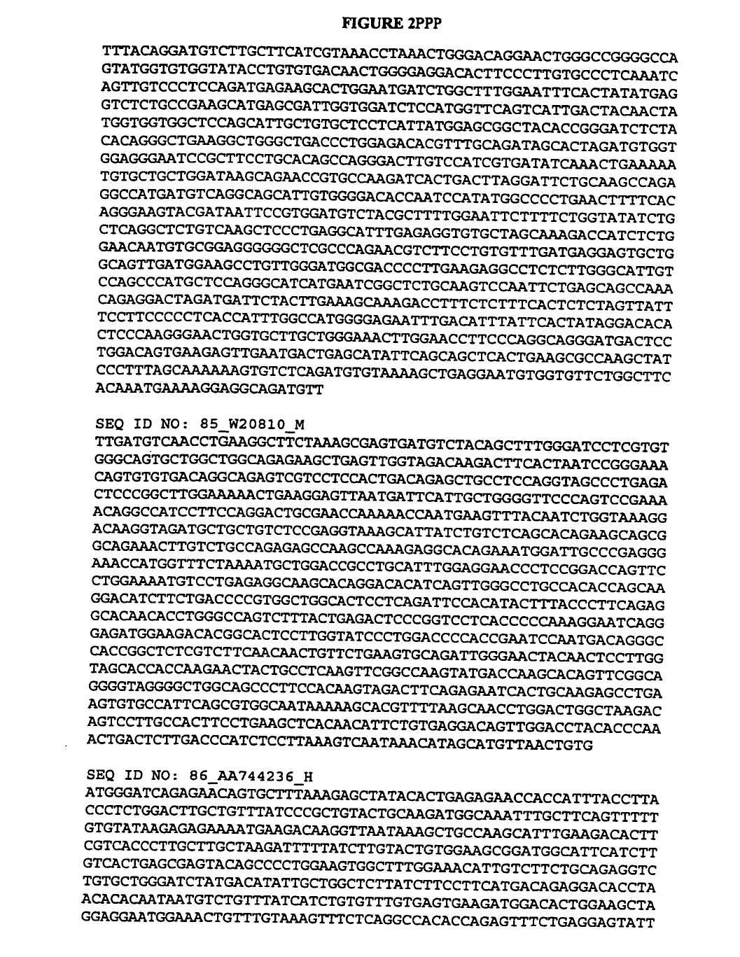

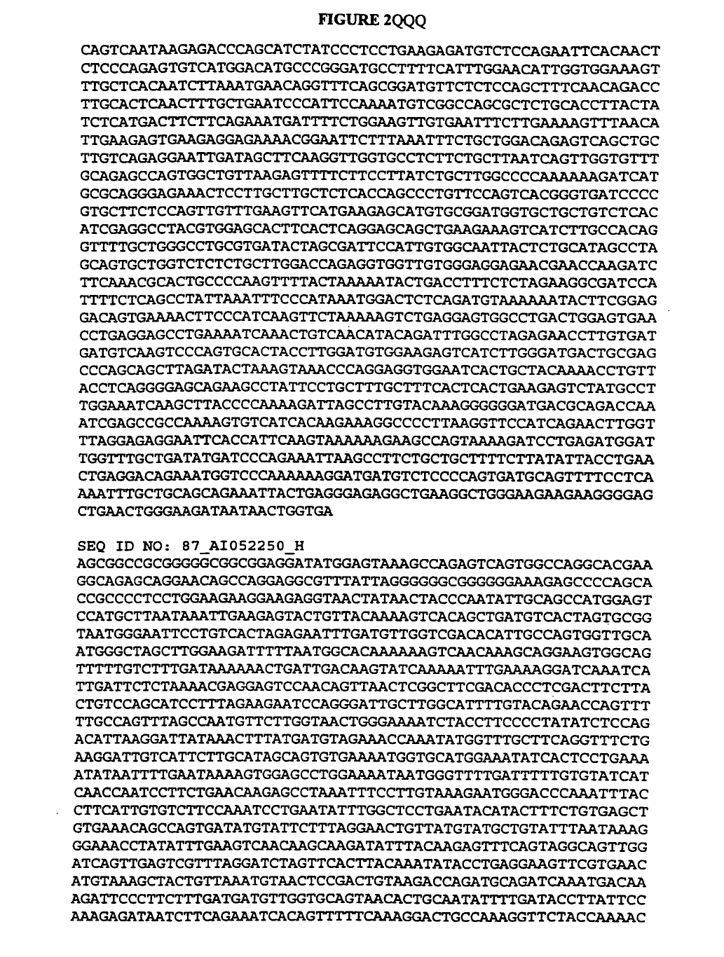

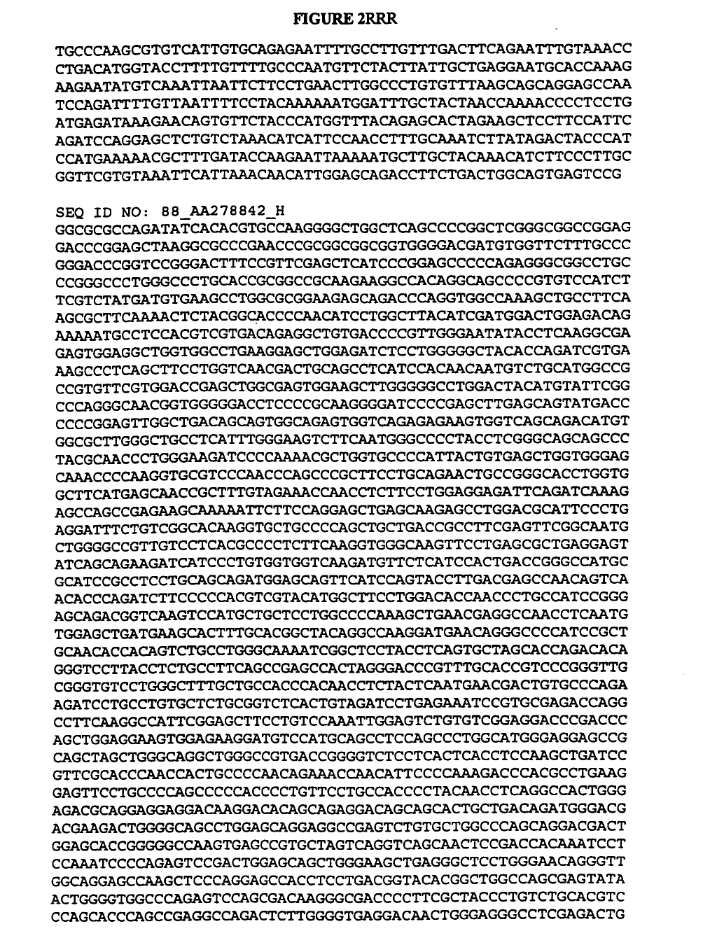

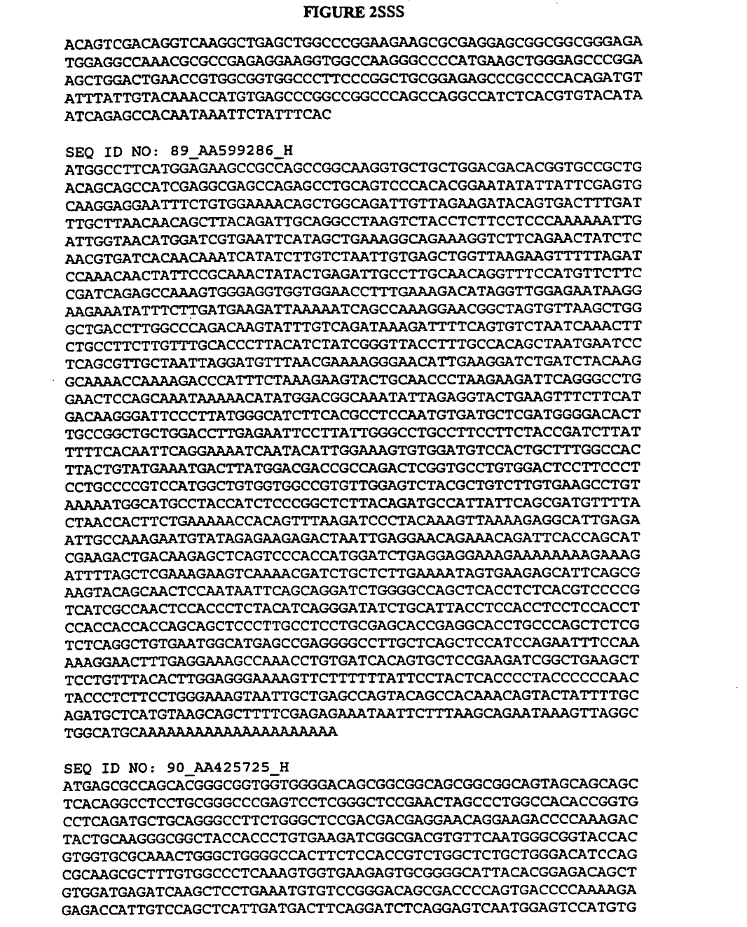

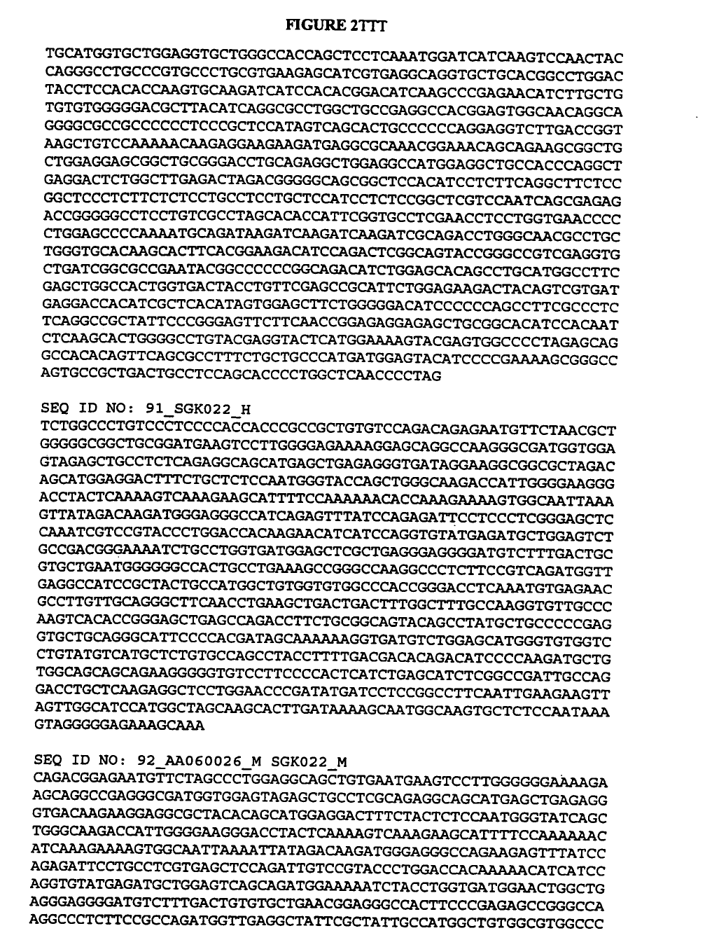

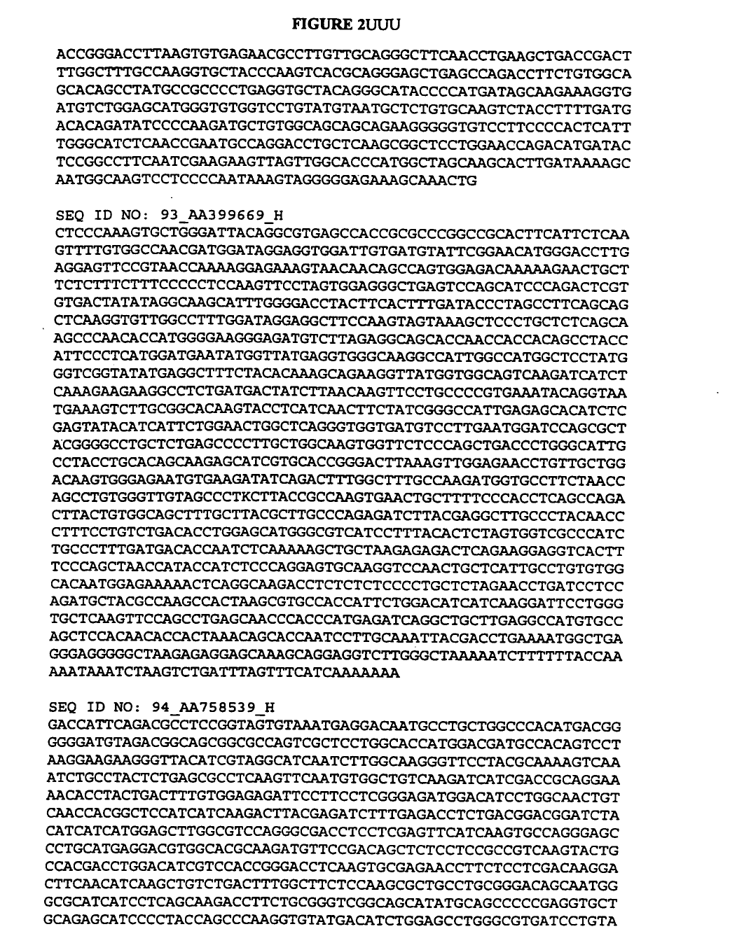

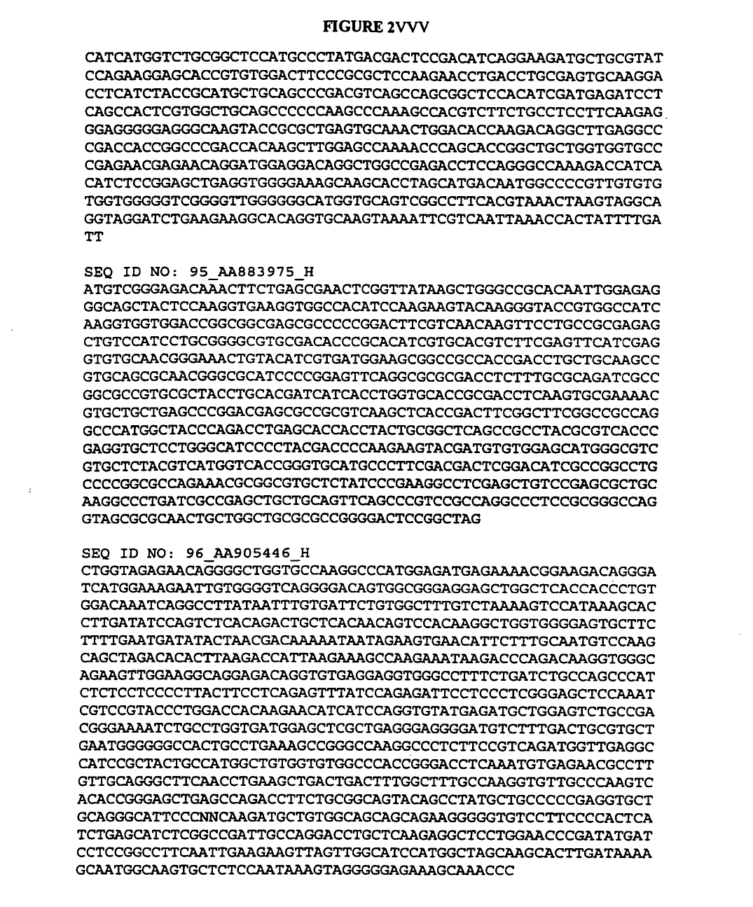

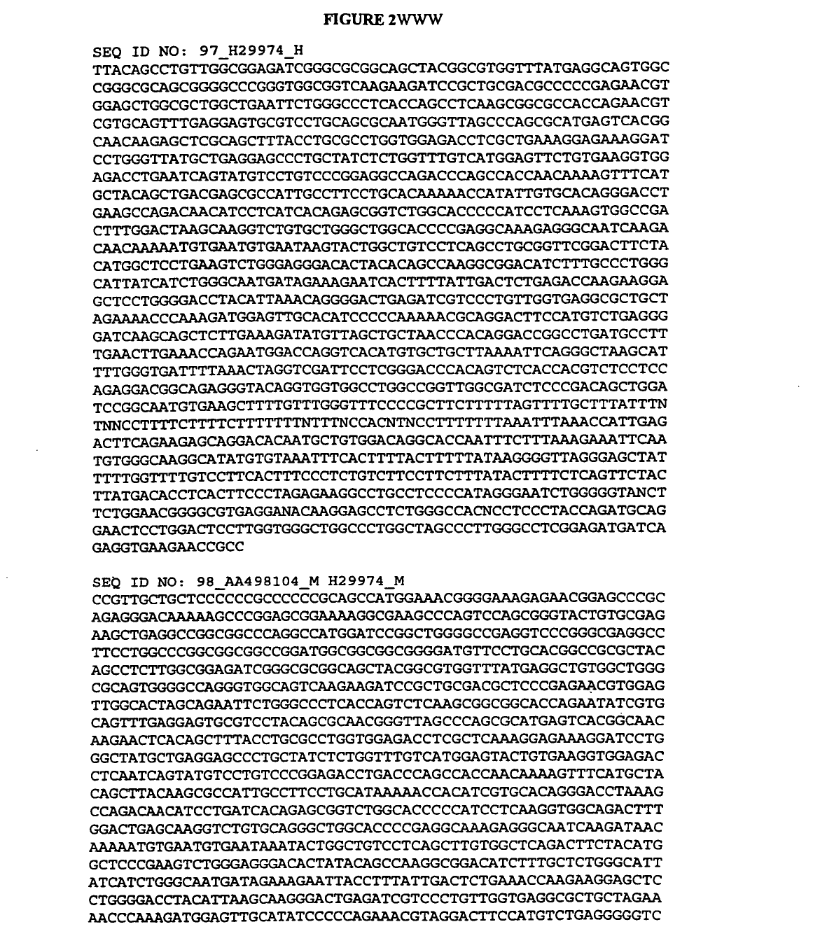

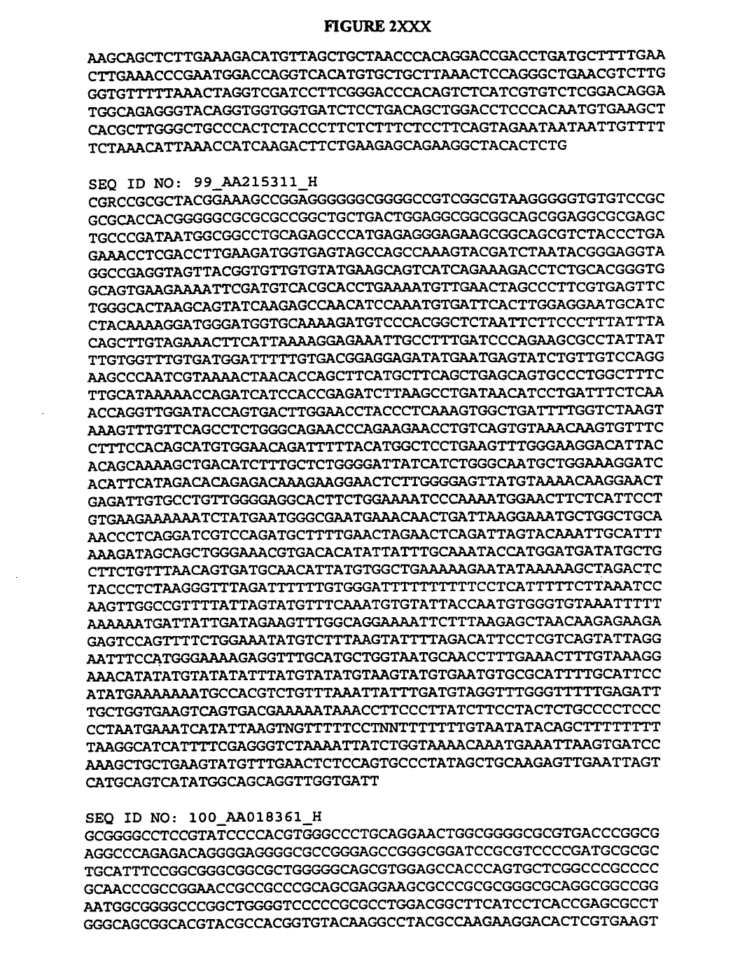

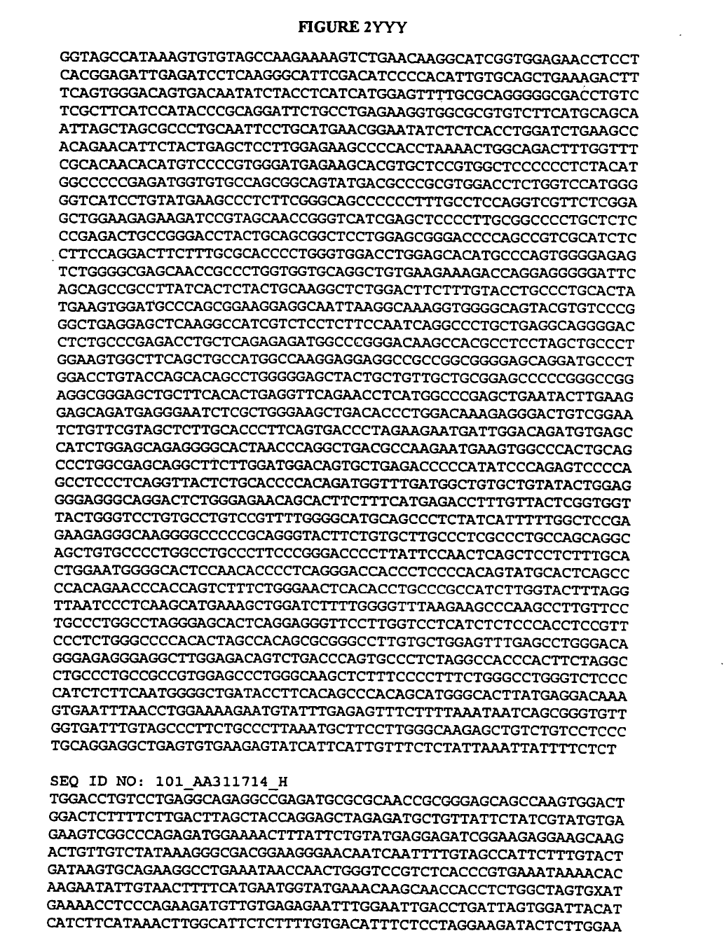

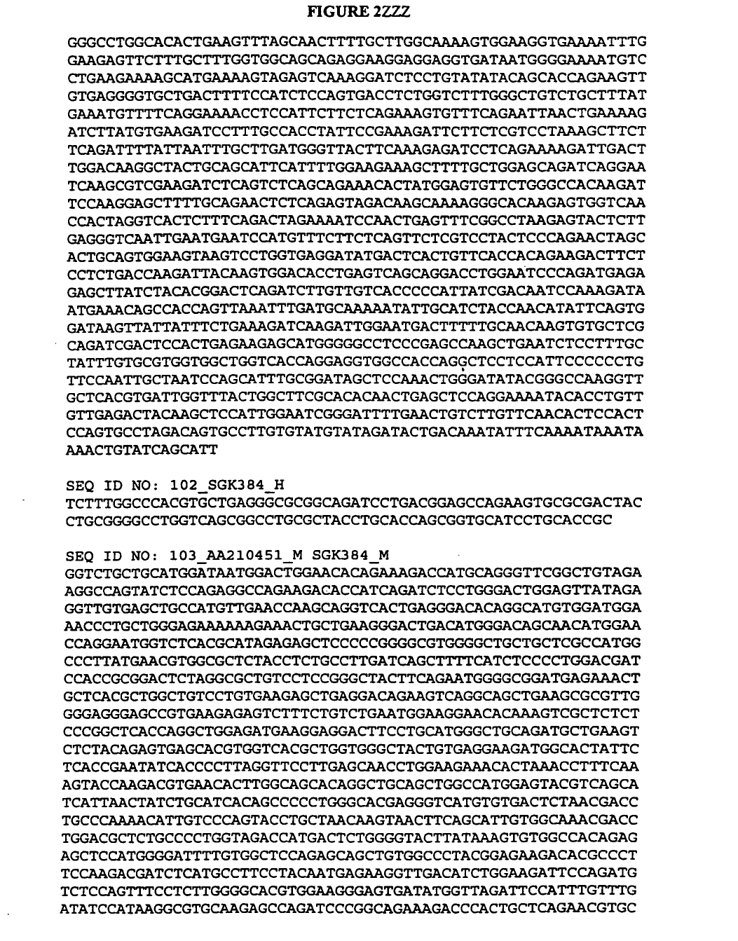

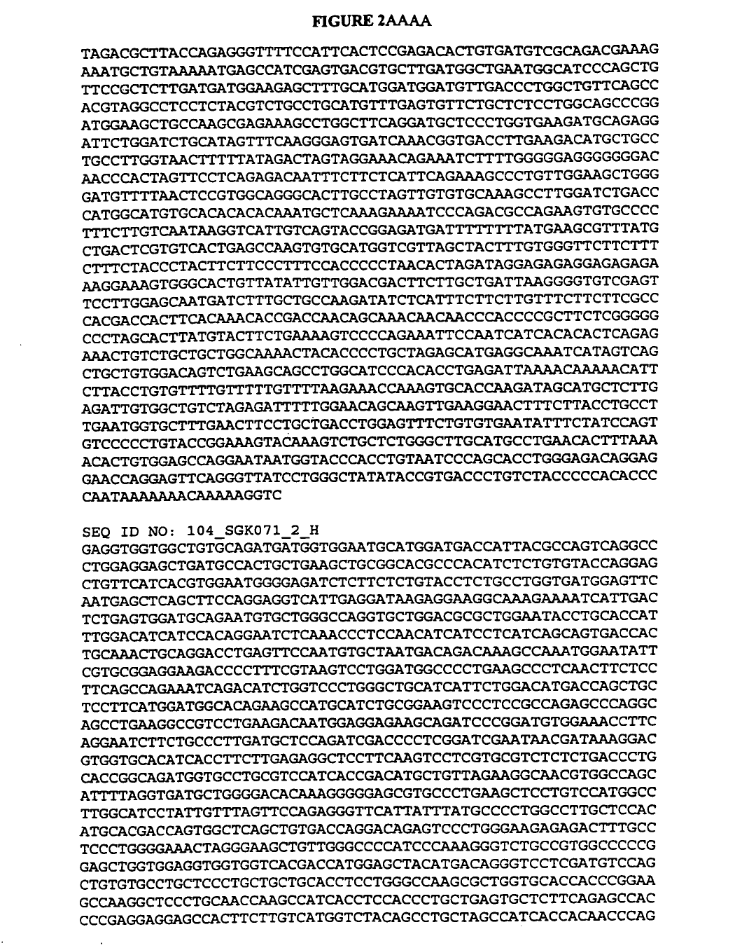

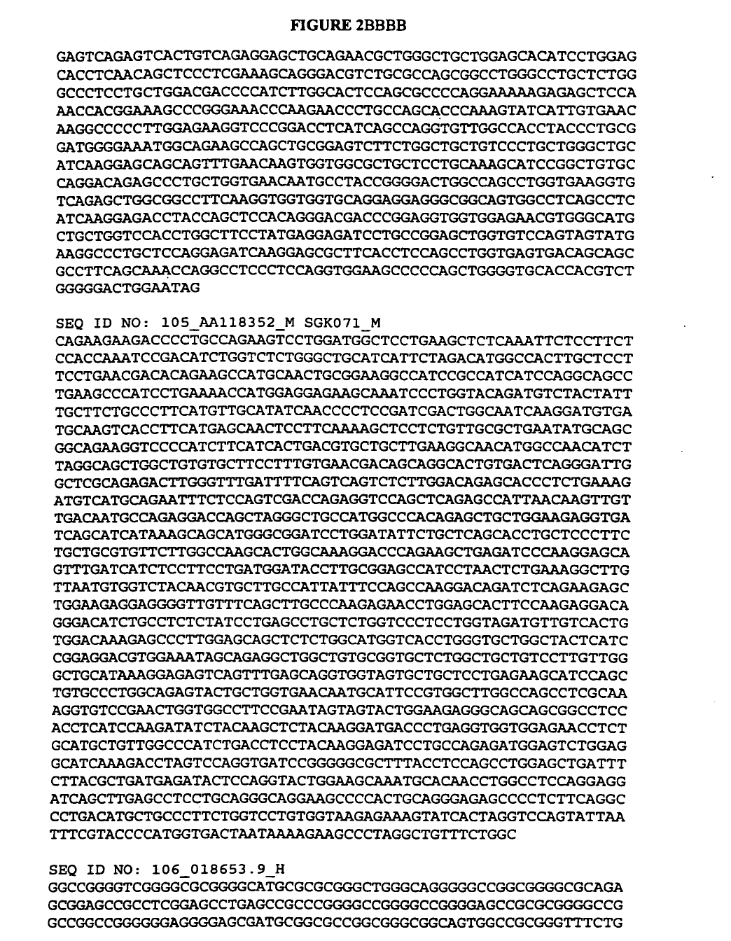

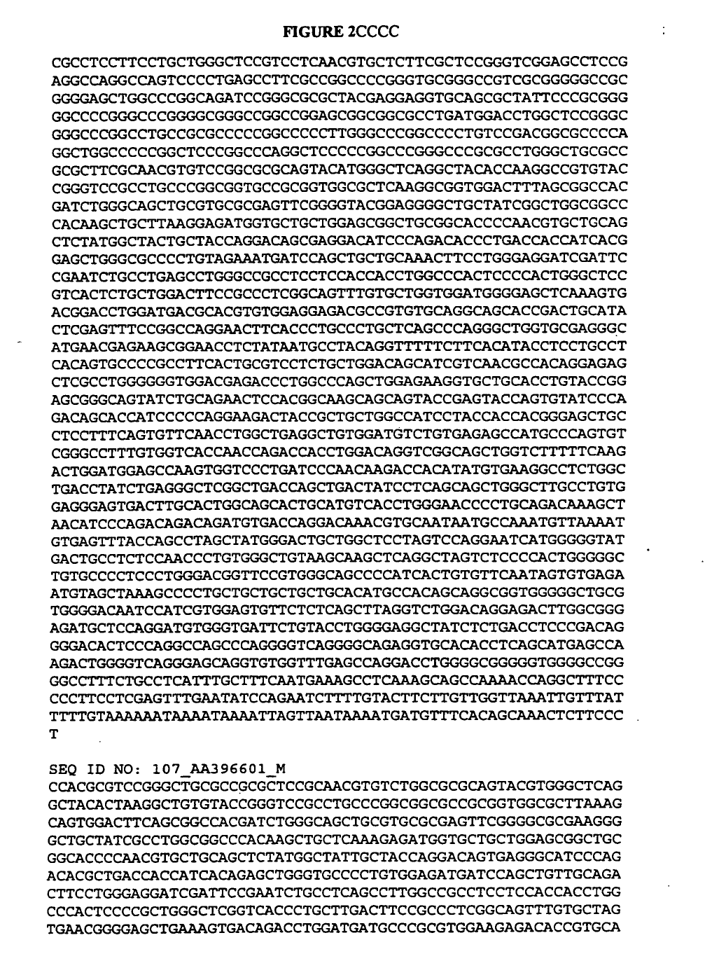

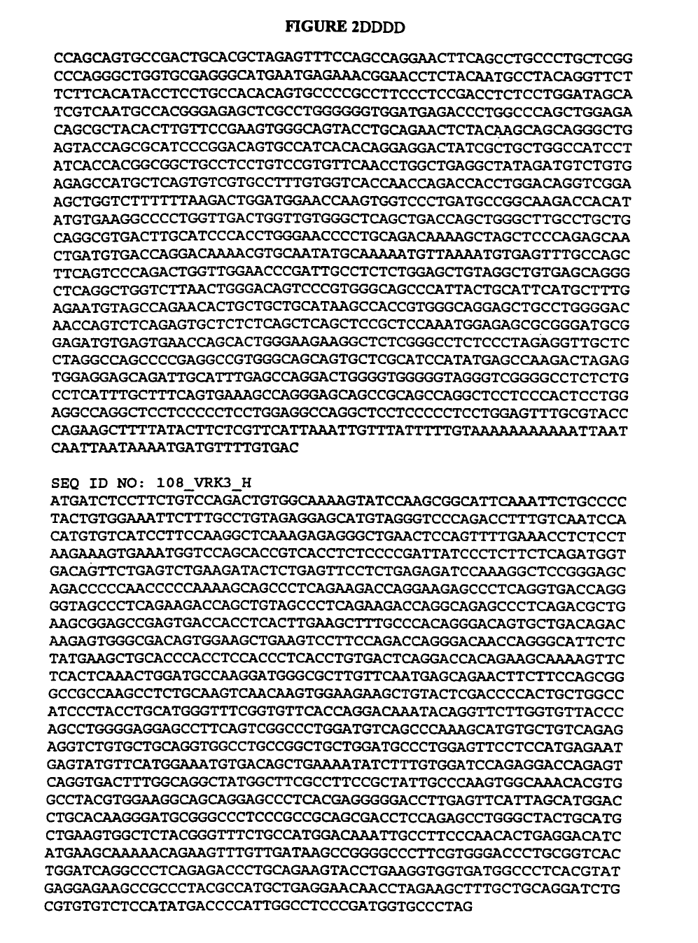

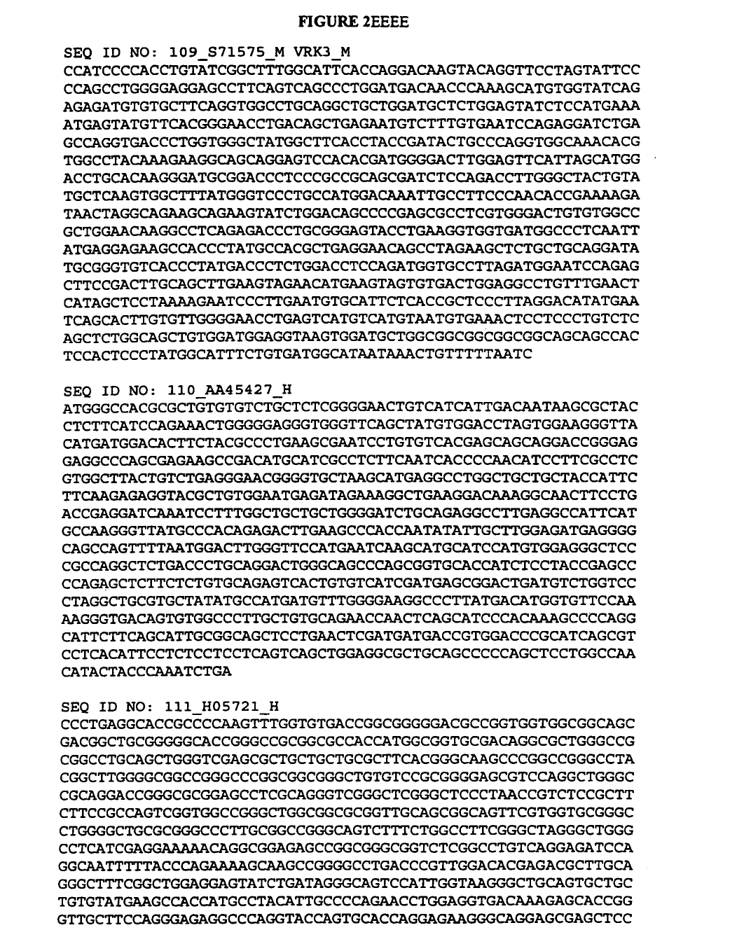

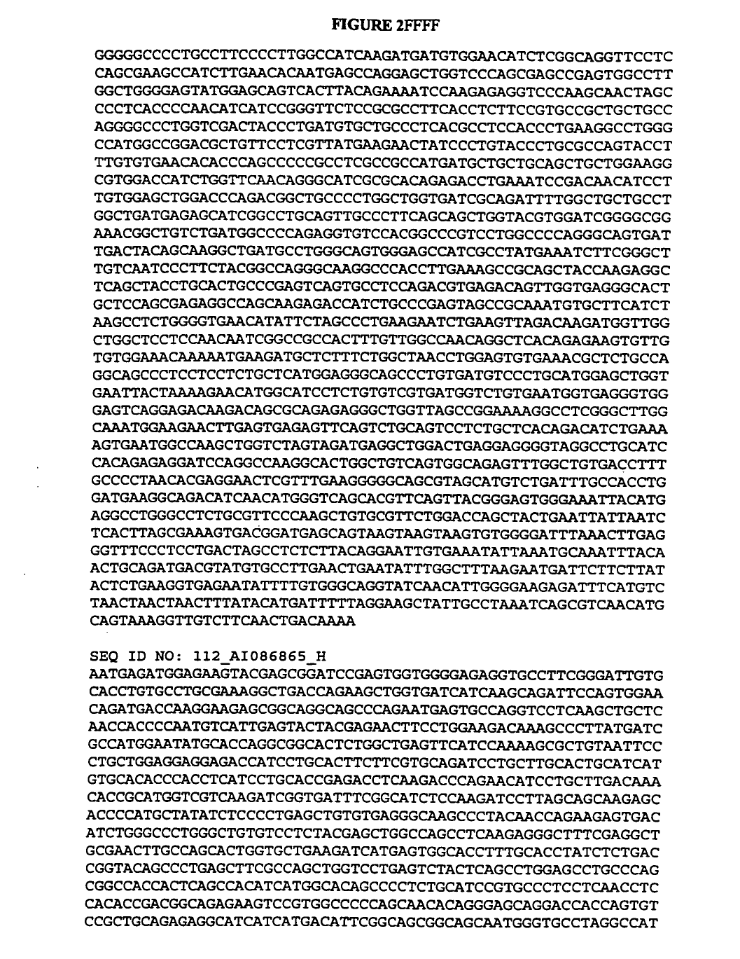

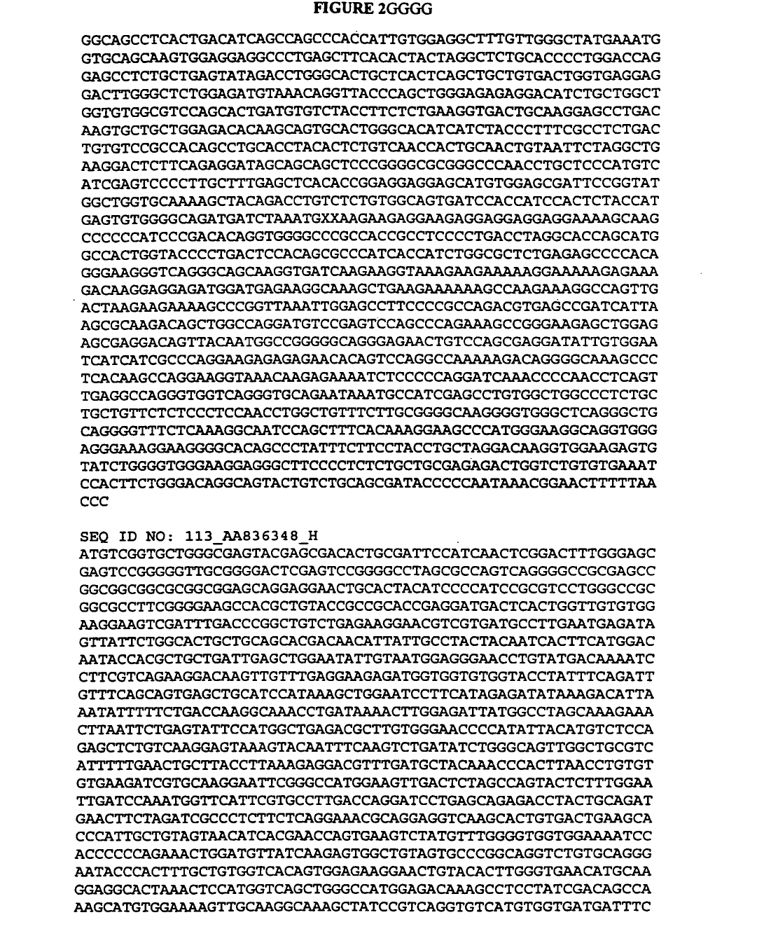

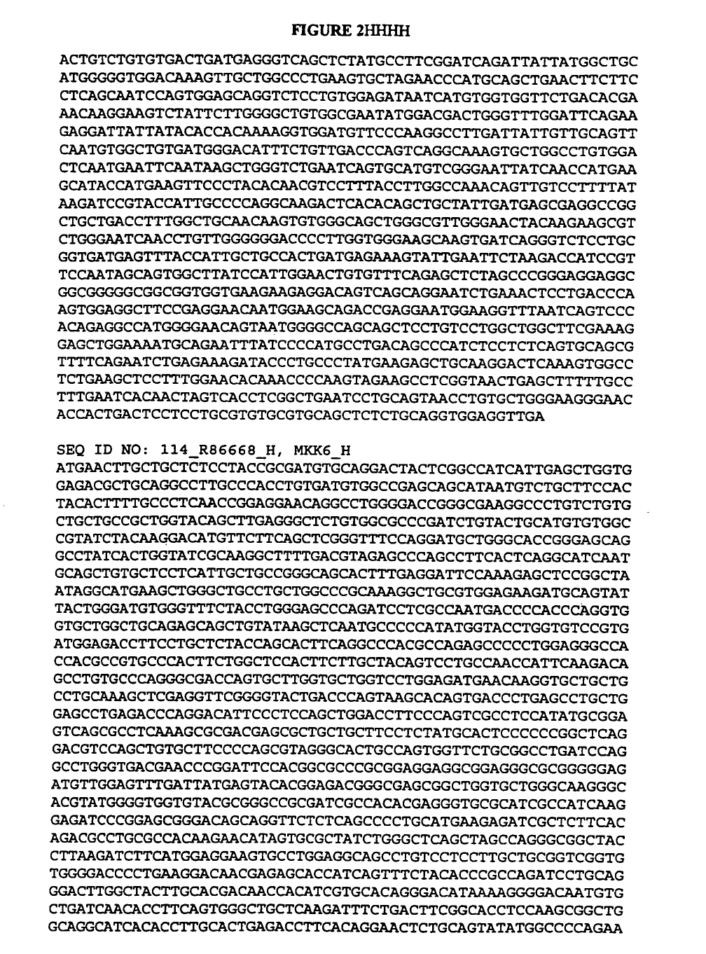

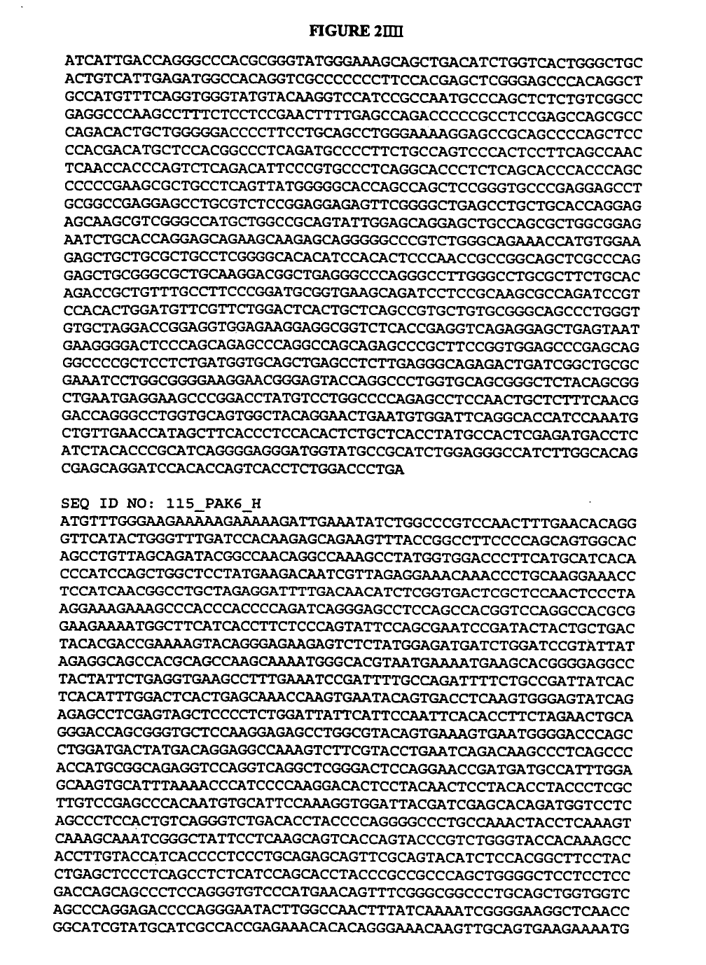

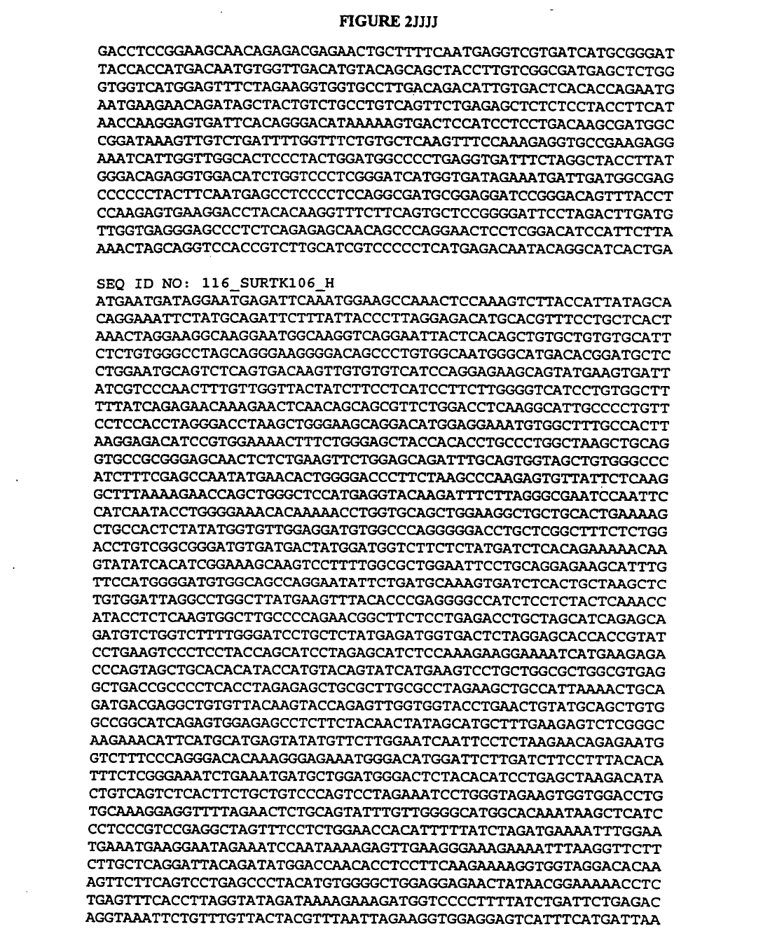

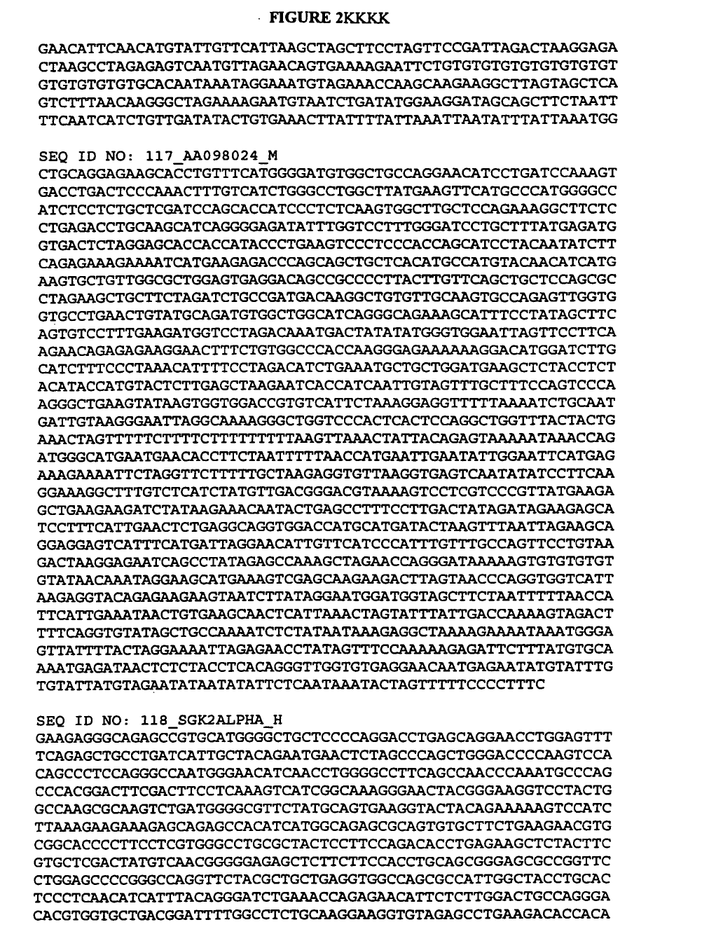

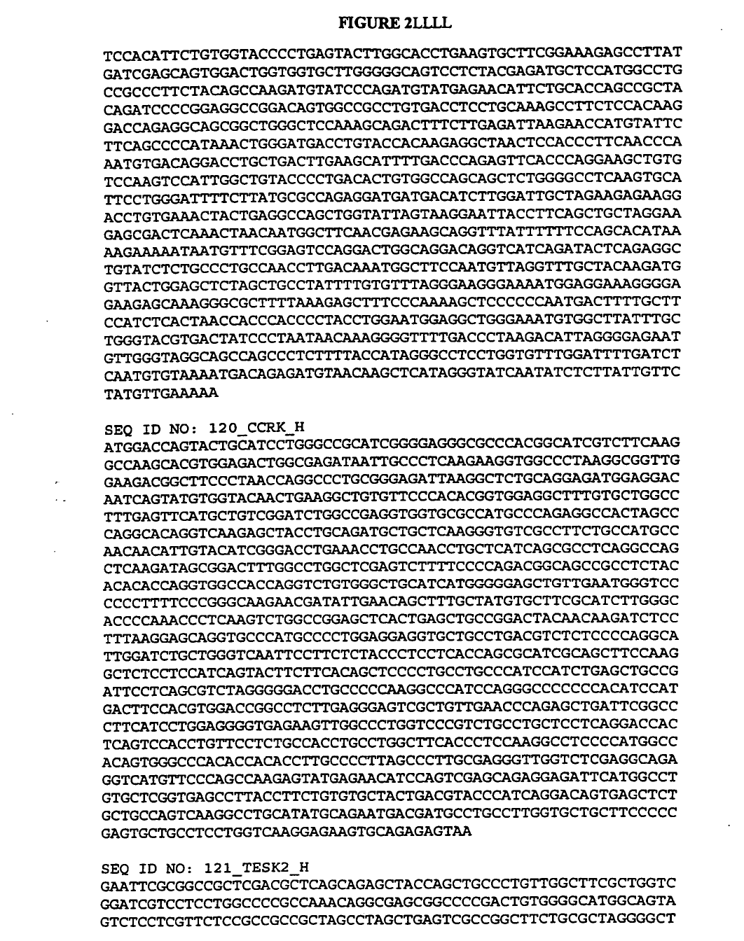

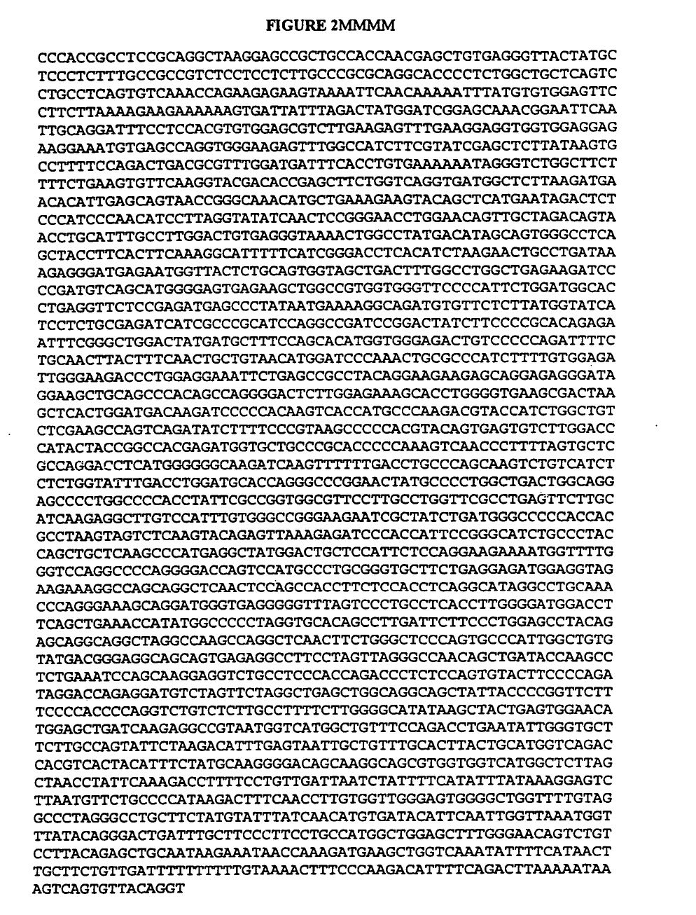

Finally, many of the nucleic acid molecules of the invention are provided as a partial sequence only (FIG. 2A through 2QQ). However, it is standard for one of ordinary skill in the art to obtain a full-length sequence when provided with a partial sequence. Similarly, when provided with a partial or full-length sequence it is standard for one of ordinary skill in the art to obtain nucleic acid sequence coding for homologous proteins. Therefore, these nucleic acid molecules are also part of the invention.

The characteristics of the protein kinase nucleic acid sequences of the invention are provided in Table 1. The protein kinases fall into 10 known groups: AGC, CAMK, CKI, CMGC, dsPK, EIFK, LIMK, MLK, STE and TK. In addition, there are a significant number of protein kinases that do not belong to any of the known groups, and therefore presumably define new protein kinase groups.

Additional characteristics may be found, inter alia, in the tables, namely Table 1, Table 2, Table 3 and Table 4, shown below.

II. Nucleic Acid Probes. Methods. and Kits for Detection of Protein Kinases

A nucleic acid probe of the present invention may be used to probe an appropriate chromosomal or cDNA library by usual hybridization methods to obtain other nucleic acid molecules of the present invention. A chromosomal DNA or cDNA library may be prepared from appropriate cells according to recognized methods in the art (cf. “Molecular Cloning: A Laboratory Manual”, second edition, Cold Spring Harbor Laboratory, Sambrook, Fritsch, & Maniatis, eds., 1989).

In the alternative, chemical synthesis can be carried out in order to obtain nucleic acid probes having nucleotide sequences that correspond to N-terminal, kinase or C-terminal portions, for example, of the amino acid sequence of the polypeptide of interest. The synthesized nucleic acid probes may be used as primers in a polymerase chain reaction (PCR) carried out in accordance with recognized PCR techniques, essentially according to PCR Protocols, “A Guide to Methods and Applications”, Academic Press, Michael, et al., eds., 1990, utilizing the appropriate chromosomal or cDNA library to obtain the fragment of the present invention.

One skilled in the art can readily design such probes based on the sequence disclosed herein using methods of computer alignment and sequence analysis known in the art (“Molecular Cloning: A Laboratory Manual”, 1989, supra). The hybridization probes of the present invention can be labeled by standard labeling techniques such as with a radiolabel, enzyme label, fluorescent label, biotin-avidin label, chemiluminescence, and the like. After hybridization, the probes may be visualized using known methods.

The nucleic acid probes of the present invention include RNA, as well as DNA probes, such probes being generated using techniques known in the art. The nucleic acid probe may be immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

The test samples suitable for nucleic acid probing methods of the present invention include, for example, cells or nucleic acid extracts of cells, or biological fluids. The samples used in the above-described methods will vary based on the assay format, the detection method and the nature of the tissues, cells or extracts to be assayed. Methods for preparing nucleic acid extracts of cells are well known in the art and can be readily adapted in order to obtain a sample that is compatible with the method utilized.

One method of detecting the presence of nucleic acids of the invention in a sample comprises (a) contacting said sample with the above-described nucleic acid probe under conditions such that hybridization occurs, and (b) detecting the presence of said probe bound to said nucleic acid molecule. One skilled in the art would select the nucleic acid probe according to techniques known in the art as described above. Samples to be tested include but should not be limited to RNA samples of human tissue.

A kit for detecting the presence of nucleic acids of the invention in a sample comprises at least one container means having disposed therein the above-described nucleic acid probe. The kit may further comprise other containers comprising one or more of the following: wash reagents and reagents capable of detecting the presence of bound nucleic acid probe. Examples of detection reagents include, but are not limited to radiolabelled probes, enzymatic labeled probes (horseradish peroxidase, alkaline phosphatase), and affinity labeled probes (biotin, avidin, or steptavidin).

In detail, a compartmentalized kit includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample, a container which contains the probe or primers used in the assay, containers which contain wash reagents (such as phosphate buffered saline, Tris-buffers, and the like), and containers which contain the reagents used to detect the hybridized probe, bound antibody, amplified product, or the like. One skilled in the art will readily recognize that the nucleic acid probes described in the present invention can readily be incorporated into one of the established kit formats that are well known in the art.

III. DNA Constructs Comprising a Protein Kinase Nucleic Acid Molecule and Cells Containing These Constructs.

The present invention also relates to a recombinant DNA molecule comprising, 5′ to 3′, a promoter effective to initiate transcription in a host cell and the above-described nucleic acid molecules. In addition, the present invention relates to a recombinant DNA molecule comprising a vector and an above-described nucleic acid molecule. The present invention also relates to a nucleic acid molecule comprising a transcriptional region functional in a cell, a sequence complementary to an RNA sequence encoding an amino acid sequence corresponding to the above-described polypeptide, and a transcriptional termination region functional in said cell. The above-described molecules may be isolated and/or purified DNA molecules.

The present invention also relates to a cell or organism that contains an above-described nucleic acid molecule and thereby is capable of expressing a polypeptide. The polypeptide may be purified from cells that have been altered to express the polypeptide. A cell is said to be “altered to express a desired polypeptide” when the cell, through genetic manipulation, is made to produce a protein which it normally does not produce or which the cell normally produces at lower levels. One skilled in the art can readily adapt procedures for introducing and expressing either genomic, cDNA, or synthetic sequences into either eukaryotic or prokaryotic cells.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene sequence expression. The precise nature of the regulatory regions needed for gene sequence expression may vary from organism to organism, but shall in general include a promoter region which, in prokaryotes, contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like.

If desired, the non-coding region 3′ to the sequence encoding a kinase of the invention may be obtained by the above-described methods. This region may be retained for its transcriptional termination regulatory sequences, such as termination and polyadenylation. Thus, by retaining the 3′-region naturally contiguous to the DNA sequence encoding a kinase of the invention, the transcriptional termination signals may be provided. Where the transcriptional termination signals are not satisfactorily functional in the expression host cell, then a 3′ region functional in the host cell may be substituted.

Two DNA sequences (such as a promoter region sequence and a sequence encoding a kinase of the invention) are said to be operably linked if the nature of the linkage between the two DNA sequences does not (1) result in the introduction of a frame-shift mutation, (2) interfere with the ability of the promoter region sequence to direct the transcription of a gene sequence encoding a kinase of the invention, or (3) interfere with the ability of the gene sequence of a kinase of the invention to be transcribed by the promoter region sequence. Thus, a promoter region would be operably linked to a DNA sequence if the promoter were capable of effecting transcription of that DNA sequence. Thus, to express a gene encoding a kinase of the invention, transcriptional and translational signals recognized by an appropriate host are necessary.

The present invention encompasses the expression of a gene encoding a kinase of the invention (or a functional derivative thereof) in either prokaryotic or eukaryotic cells. Prokaryotic hosts are, generally, very efficient and convenient for the production of recombinant proteins and are, therefore, one type of preferred expression system for kinases of the invention. Prokaryotes most frequently are represented by various strains of E. coli. However, other microbial strains may also be used, including other bacterial strains.

In prokaryotic systems, plasmid vectors that contain replication sites and control sequences derived from a species compatible with the host may be used. Examples of suitable plasmid vectors may include pBR322, pUC118, pUC 119 and the like; suitable phage or bacteriophage vectors may include γgt10, γgt11 and the like; and suitable virus vectors may include pMAM-neo, pKRC and the like. Preferably, the selected vector of the present invention has the capacity to replicate in the selected host cell.

Recognized prokaryotic hosts include bacteria such as E. coli, Bacillus, Streptomyces, Pseudomonas, Salmonella, Serratia, and the like. However, under such conditions, the polypeptide will not be glycosylated. The prokaryotic host must be compatible with the replicon and control sequences in the expression plasmid.

To express a kinase of the invention (or a functional derivative thereof) in a prokaryotic cell, it is necessary to operably link the sequence encoding the kinase of the invention to a functional prokaryotic promoter. Such promoters may be either constitutive or, more preferably, regulatable (ie., inducible or derepressible). Examples of constitutive promoters include the int promoter of bacteriophage λ, the bla promoter of the β-lactamase gene sequence of pBR322, and the cat promoter of the chloramphenicol acetyl transferase gene sequence of pPR325, and the like. Examples of inducible prokaryotic promoters include the major right and left promoters of bacteriophage λ (P_{L} and P_{R}), the trp, recA, λacZ, λacI, and gal promoters of E. coli, the λ-amylase (Ulmanen et al., J. Bacteriol. 162:176-182, 1985) and the ç-28-specific promoters of B. subtilis (Gilman et al., Gene Sequence 32:11-20, 1984), the promoters of the bacteriophages of Bacillus (Gryczan, In: The Molecular Biology of the Bacilli, Academic Press, Inc., NY, 1982), and Streptomyces promoters (Ward et al., Mol. Gen. Genet. 203:468-478, 1986). Prokaryotic promoters are reviewed by Glick (Ind. Microbiot. 1:277-282, 1987), Cenatiempo (Biochimie 68:505-516, 1986), and Gottesman (Ann. Rev. Genet. 18:415-442, 1984).

Proper expression in a prokaryotic cell also requires the presence of a ribosome-binding site upstream of the gene sequence-encoding sequence. Such ribosome-binding sites are disclosed, for example, by Gold et al. (Ann. Rev. Microbiol. 35:365-404, 1981). The selection of control sequences, expression vectors, transformation methods, and the like, are dependent on the type of host cell used to express the gene. As used herein, “cell”, “cell line”, and “cell culture” may be used interchangeably and all such designations include progeny. Thus, the words “transformants” or “transformed cells” include the primary subject cell and cultures derived therefrom, without regard to the number of transfers. It is also understood that all progeny may not be precisely identical in DNA content, due to deliberate or inadvertent mutations. However, as defined, mutant progeny have the same functionality as that of the originally transformed cell.

Host cells which may be used in the expression systems of the present invention are not strictly limited, provided that they are suitable for use in the expression of the kinase polypeptide of interest. Suitable hosts may often include eukaryotic cells. Preferred eukaryotic hosts include, for example, yeast, fungi, insect cells, mammalian cells either in vivo, or in tissue culture. Mammalian cells which may be useful as hosts include HeLa cells, cells of fibroblast origin such as VERO or CHO-K1, or cells of lymphoid origin and their derivatives. Preferred mammalian host cells include SP2/0 and J558L, as well as neuroblastoma cell lines such as IMR 332, which may provide better capacities for correct post-translational processing.

In addition, plant cells are also available as hosts, and control sequences compatible with plant cells are available, such as the cauliflower mosaic virus 35S and 19S, and nopaline synthase promoter and polyadenylation signal sequences. Another preferred host is an insect cell, for example the Drosophila larvae. Using insect cells as hosts, the Drosophila alcohol dehydrogenase promoter can be used (Rubin, Science 240:1453-1459, 1988). Alternatively, baculovirus vectors can be engineered to express large amounts of kinases of the invention in insect cells (Jasny, Science 238:1653, 1987; Miller et al., In: Genetic Engineering, Vol. 8, Plenum, Setlow et al., eds., pp. 277-297, 1986).

Any of a series of yeast expression systems can be utilized which incorporate promoter and termination elements from the actively expressed sequences coding for glycolytic enzymes that are produced in large quantities when yeast are grown in mediums rich in glucose. Known glycolytic gene sequences can also provide very efficient transcriptional control signals. Yeast provides substantial advantages in that it can also carry out post-translational modifications. A number of recombinant DNA strategies exist utilizing strong promoter sequences and high copy number plasmids which can be utilized for production of the desired proteins in yeast. Yeast recognizes leader sequences on cloned mammalian genes and secretes peptides bearing leader sequences (i.e., pre-peptides). Several possible vector systems are available for the expression of kinases of the invention in a mammalian host.

A wide variety of transcriptional and translational regulatory sequences may be employed, depending upon the nature of the host. The transcriptional and translational regulatory signals may be derived from viral sources, such as adenovirus, bovine papilloma virus, cytomegalovirus, simian virus, or the like, where the regulatory signals are associated with a particular gene sequence which has a high level of expression. Alternatively, promoters from mammalian expression products, such as actin, collagen, myosin, and the like, may be employed. Transcriptional initiation regulatory signals may be selected which allow for repression or activation, so that expression of the gene sequences can be modulated. Of interest are regulatory signals which are temperature-sensitive so that by varying the temperature, expression can be repressed or initiated, or are subject to chemical (such as metabolite) regulation.

Expression of kinases of the invention in eukaryotic hosts requires the use of eukaryotic regulatory regions. Such regions will, in general, include a promoter region sufficient to direct the initiation of RNA synthesis. Preferred eukaryotic promoters include, for example, the promoter of the mouse metallothionein I gene sequence (Hamer et al., J. Mol. Appl. Gen. 1:273-288, 1982); the TK promoter of Herpes virus (McKnight, Cell 31:355-365, 1982); the SV40 early promoter (Benoist et al., Nature (London) 290:304-31, 1981); and the yeast gal4 gene sequence promoter (Johnston et al., Proc. Natl. Acad. Sci. (USA) 79:6971-6975, 1982; Silver et al., Proc. Natl. Acad. Sci. (USA) 81:5951-5955, 1984).

Translation of eukaryotic mRNA is initiated at the codon that encodes the first methionine. For this reason, it is preferable to ensure that the linkage between a eukaryotic promoter and a DNA sequence which encodes a kinase of the invention (or a functional derivative thereof) does not contain any intervening codons which are capable of encoding a methionine (i.e., AUG). The presence of such codons results either in the formation of a fusion protein (if the AUG codon is in the same reading frame as the kinase of the invention coding sequence) or a frame-shift mutation (if the AUG codon is not in the same reading frame as the kinase of the invention coding sequence).

A nucleic acid molecule encoding a kinase of the invention and an operably linked promoter may be introduced into a recipient prokaryotic or eukaryotic cell either as a nonreplicating DNA or RNA molecule, which may either be a linear molecule or, more preferably, a closed covalent circular molecule. Since such molecules are incapable of autonomous replication, the expression of the gene may occur through the transient expression of the introduced sequence. Alternatively, permanent expression may occur through the integration of the introduced DNA sequence into the host chromosome.

A vector may be employed which is capable of integrating the desired gene sequences into the host cell chromosome. Cells which have stably integrated the introduced DNA into their chromosomes can be selected by also introducing one or more markers which allow for selection of host cells which contain the expression vector. The marker may provide for prototrophy to an auxotrophic host, biocide resistance, e.g., antibiotics, or heavy metals, such as copper, or the like. The selectable marker gene sequence can either be directly linked to the DNA gene sequences to be expressed, or introduced into the same cell by co-transfection. Additional elements may also be needed for optimal synthesis of mRNA. These elements may include splice signals, as well as transcription promoters, enhancers, and termination signals. cDNA expression vectors incorporating such elements include those described by Okayama (Mol. Cell. Biol. 3:280-, 1983).

The introduced nucleic acid molecule can be incorporated into a plasmid or viral vector capable of autonomous replication in the recipient host. Any of a wide variety of vectors may be employed for this purpose. Factors of importance in selecting a particular plasmid or viral vector include: the ease with which recipient cells that contain the vector may be recognized and selected from those recipient cells which do not contain the vector, the number of copies of the vector which are desired in a particular host; and whether it is desirable to be able to “shuttle” the vector between host cells of different species.

Preferred prokaryotic vectors include plasmids such as those capable of replication in E. coli (such as, for example, pBR322, ColEl, pSC101, pACYC 184, πVX; “Molecular Cloning: A Laboratory Manual”, 1989, supra). Bacillus plasmids include pC194, pC221, pT127, and the like (Gryczan, In: The Molecular Biology of the Bacilli, Academic Press, NY, pp. 307-329, 1982). Suitable Streptomyces plasmids include p1J101 (Kendall et al., J. Bacteriol. 169:41774183, 1987), and streptomyces bacteriophages such as φC31 (Chater et al., In: Sixth International Symposium on Actinomycetales Biology, Akademiai Kaido, Budapest, Hungary, pp. 45-54, 1986). Pseudomonas plasmids are reviewed by John et al. (Rev. Infect. Dis. 8:693-704, 1986), and Izaki (Jpn. J. Bacteriol. 33:729-742, 1978).

Preferred eukaryotic plasmids include, for example, BPV, vaccinia, SV40, 2-micron circle, and the like, or their derivatives. Such plasmids are well known in the art (Botstein et al., Miarni Wntr. Symp. 19:265-274, 1982; Broach, In: “The Molecular Biology of the Yeast Saccharomyces: Life Cycle and Inheritance”, Cold Spring Harbor Laboratory, Cold Spring Harbor, N.Y., p. 445-470, 1981; Broach, Cell 28:203-204, 1982; Bollon et al., J. Clin. Hematol. Oncol. 10:39-48, 1980; Maniatis, In: Cell Biology: A Comprehensive Treatise, Vol. 3, Gene Sequence Expression, Academic Press, N.Y., pp. 563-608, 1980).

Once the vector or nucleic acid molecule containing the construct(s) has been prepared for expression, the DNA construct(s) may be introduced into an appropriate host cell by any of a variety of suitable means, i.e., transformation, transfection, conjugation, protoplast fusion, electroporation, particle gun technology, calcium phosphate-precipitation, direct microinjection, and the like. After the introduction of the vector, recipient cells are grown in a selective medium, which selects for the growth of vector-containing cells. Expression of the cloned gene(s) results in the production of a kinase of the invention, or fragments thereof. This can take place in the transformed cells as such, or following the induction of these cells to differentiate (for example, by administration of bromodeoxyuracil to neuroblastoma cells or the like). A variety of incubation conditions can be used to form the peptide of the present invention. The most preferred conditions are those which mimic physiological conditions.

IV. The Proteins of the Invention

A variety of methodologies known in the art can be utilized to obtain the polypeptides of the present invention. The polypeptides may be purified from tissues or cells that naturally produce the polypeptides. Alternatively, the above-described isolated nucleic acid fragments could be used to express the kinases of the invention in any organism. The samples of the present invention include cells, protein extracts or membrane extracts of cells, or biological fluids. The samples will vary based on the assay format, the detection method, and the nature of the tissues, cells or extracts used as the sample.

Any eukaryotic organism can be used as a source for the polypeptides of the invention, as long as the source organism naturally contains such polypeptides. As used herein, “source organism” refers to the original organism from which the amino acid sequence of the subunit is derived, regardless of the organism the subunit is expressed in and ultimately isolated from.

One skilled in the art can readily follow known methods for isolating proteins in order to obtain the polypeptides free of natural contaminants. These include, but are not limited to: size-exclusion chromatography, HPLC, ion-exchange chromatography, and immuno-affinity chromatography.

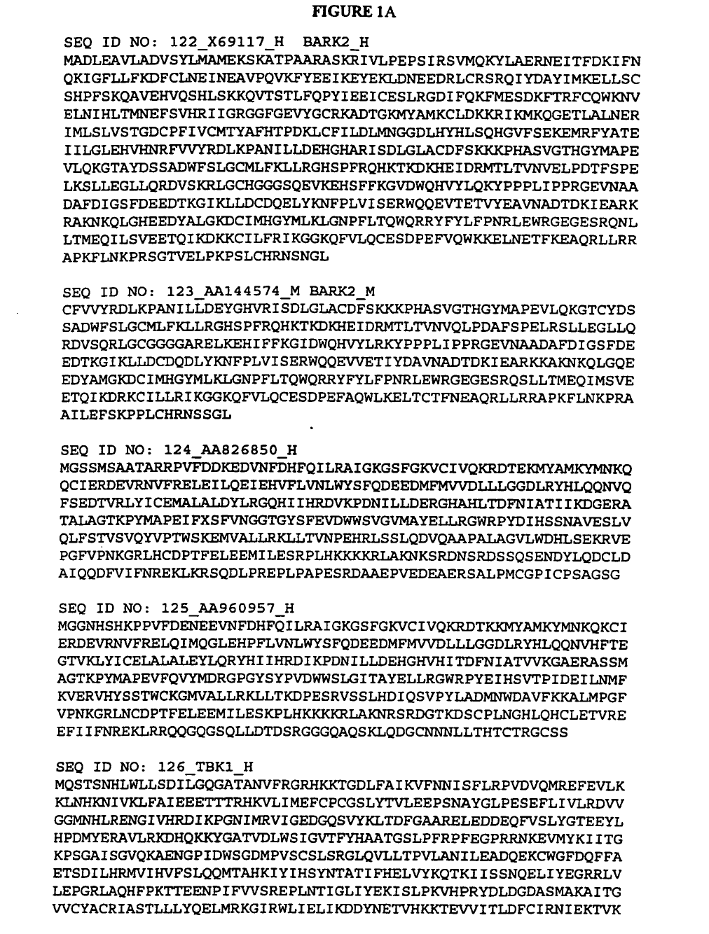

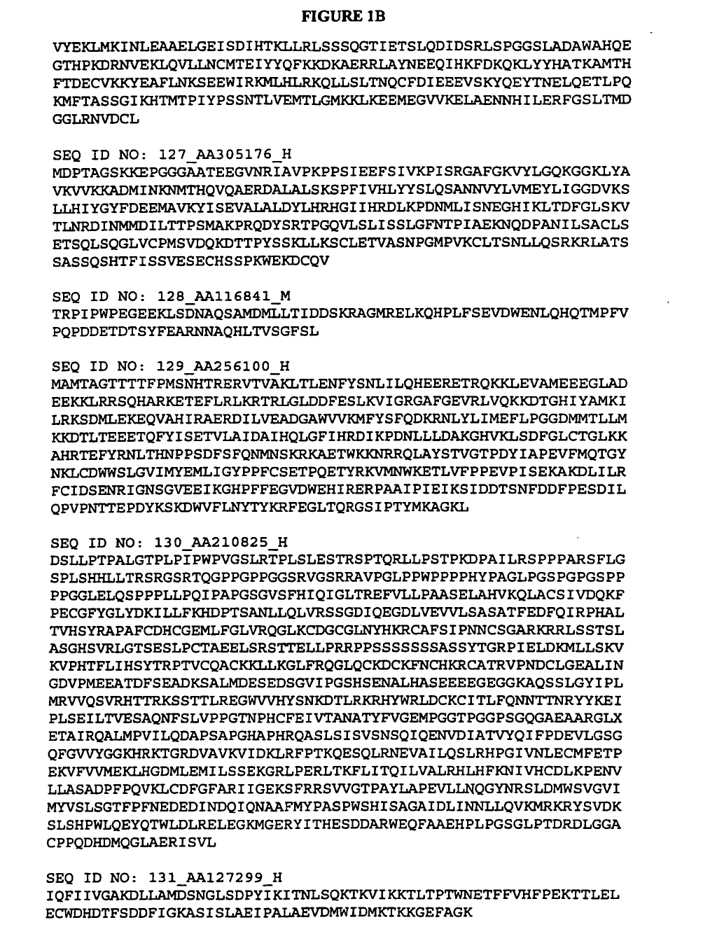

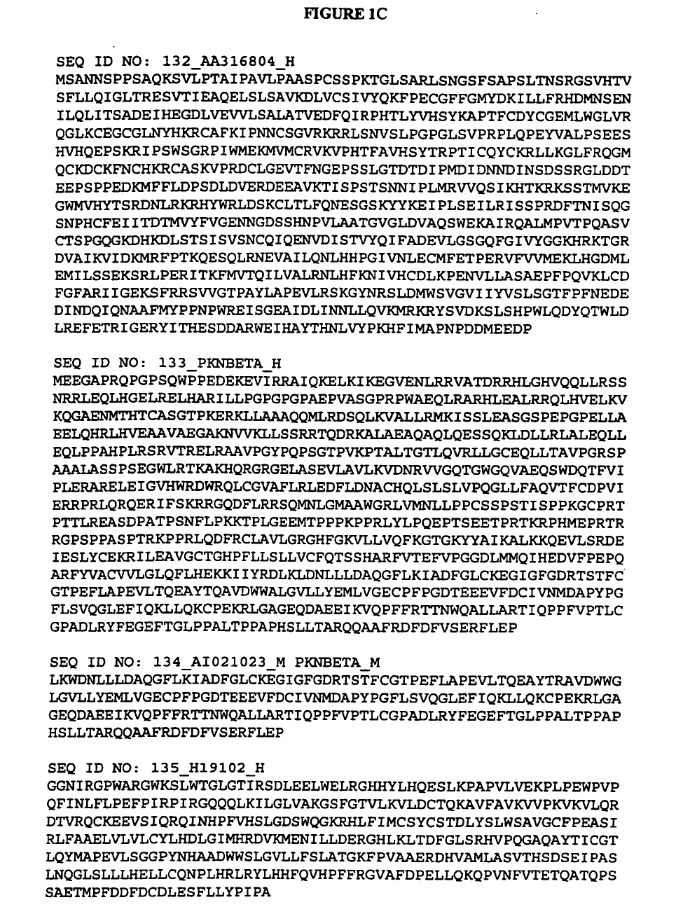

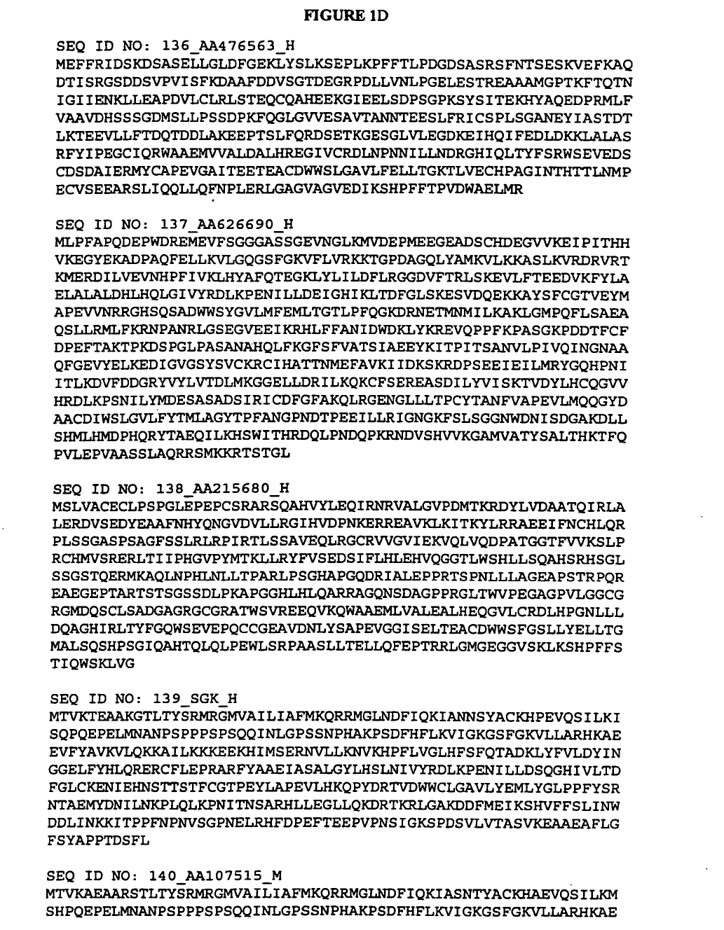

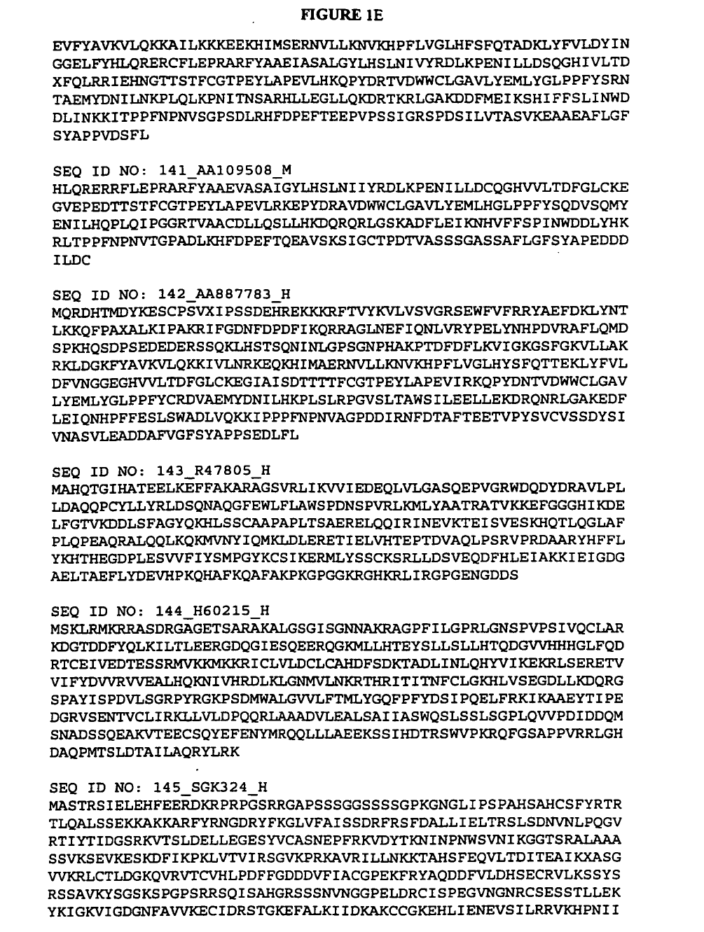

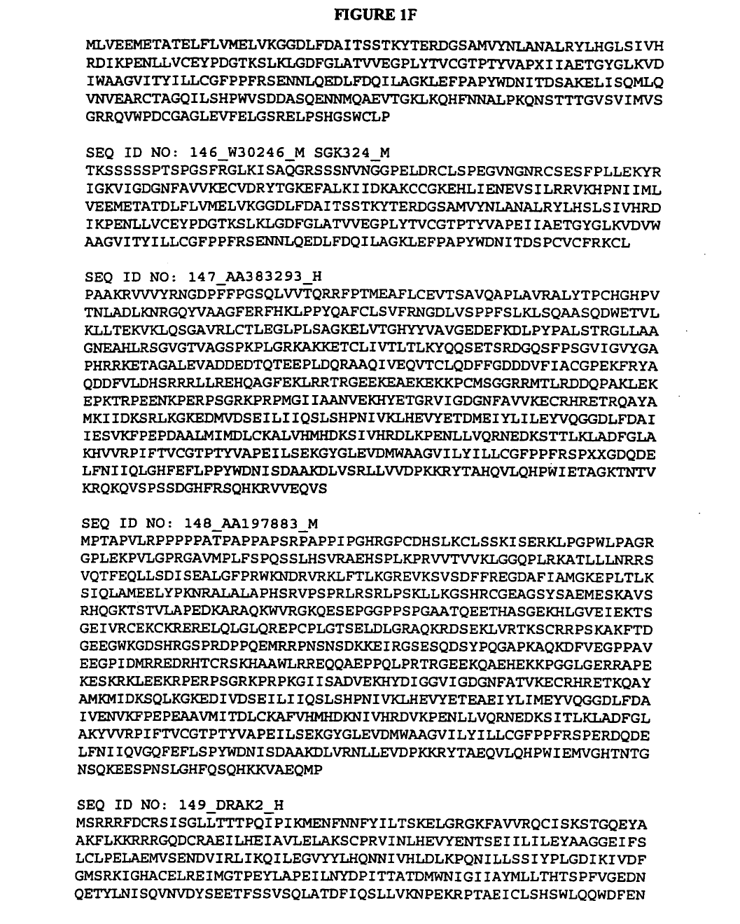

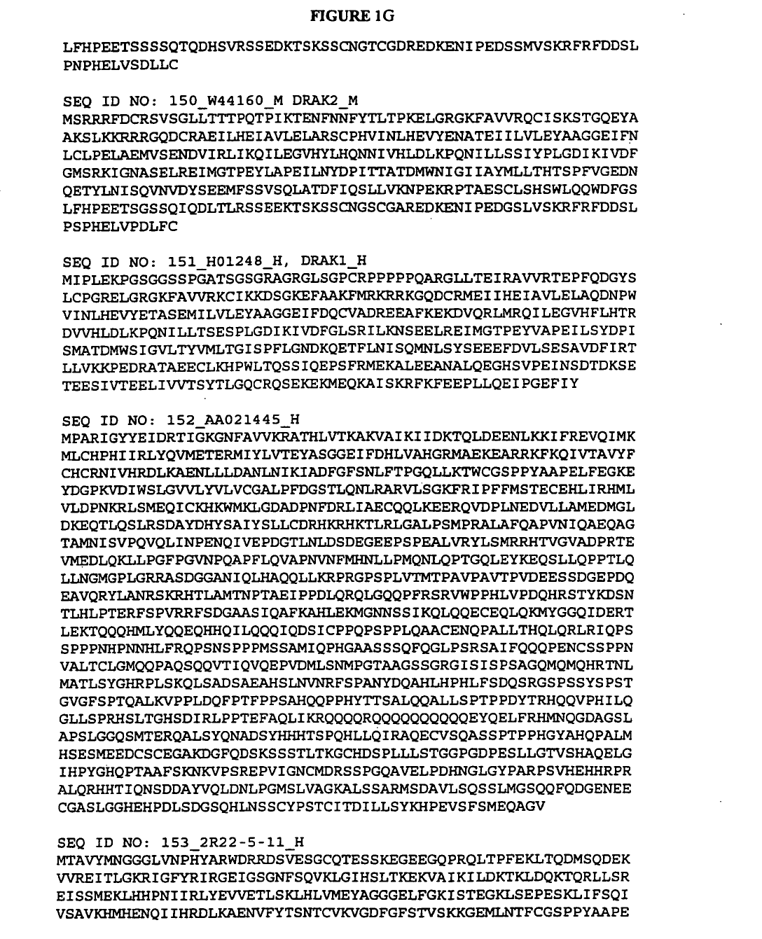

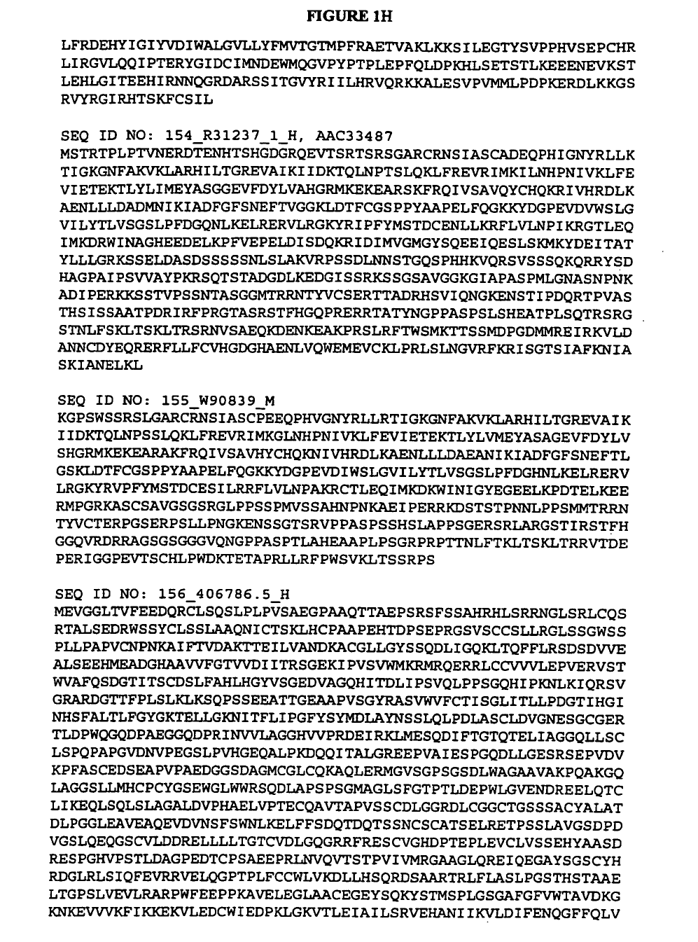

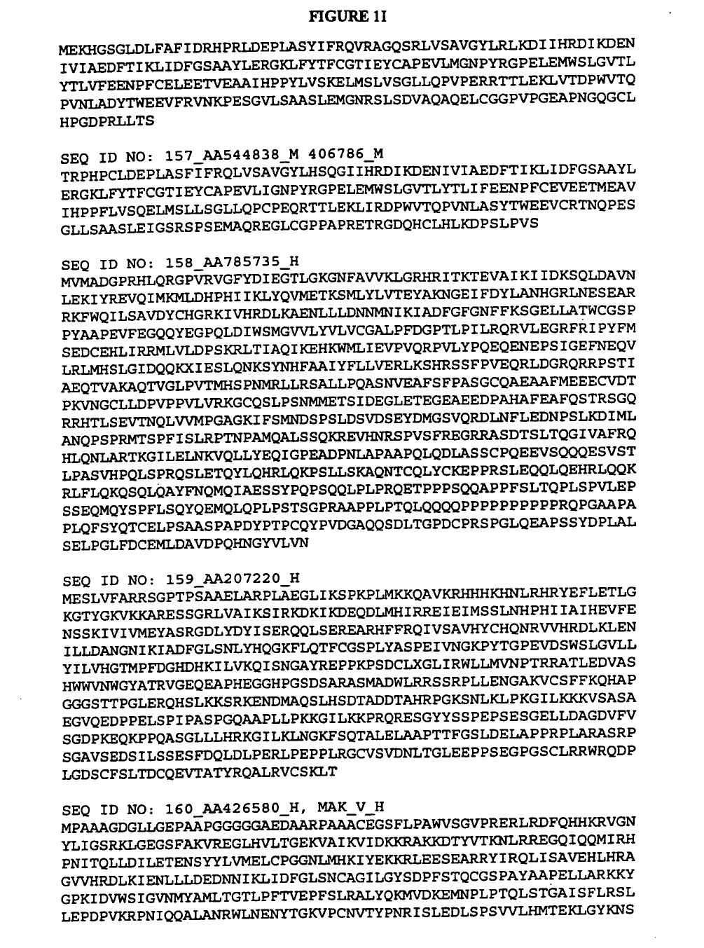

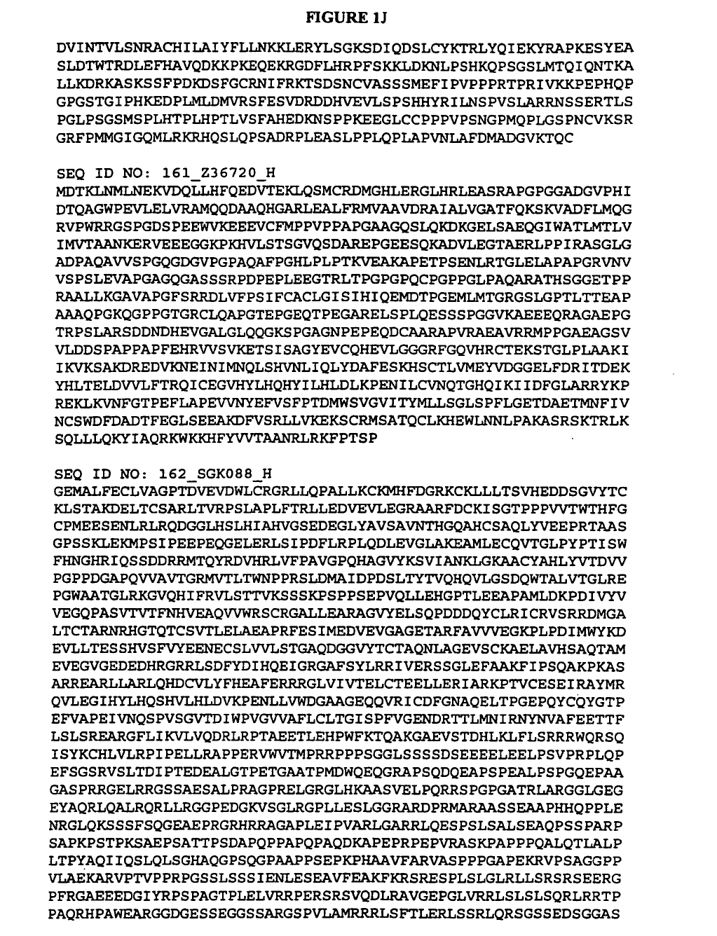

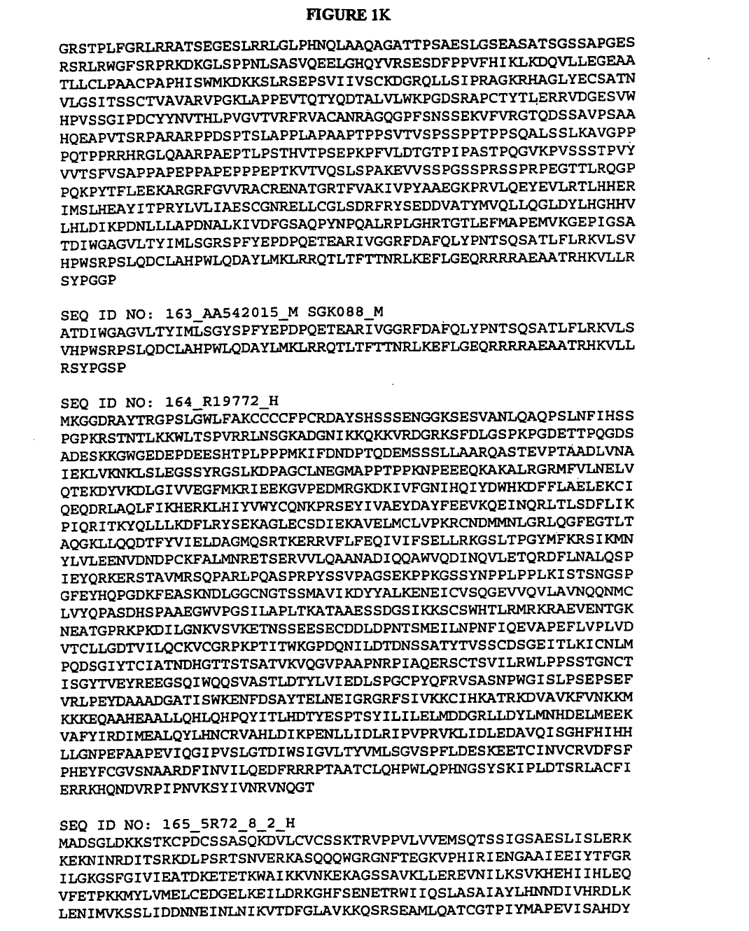

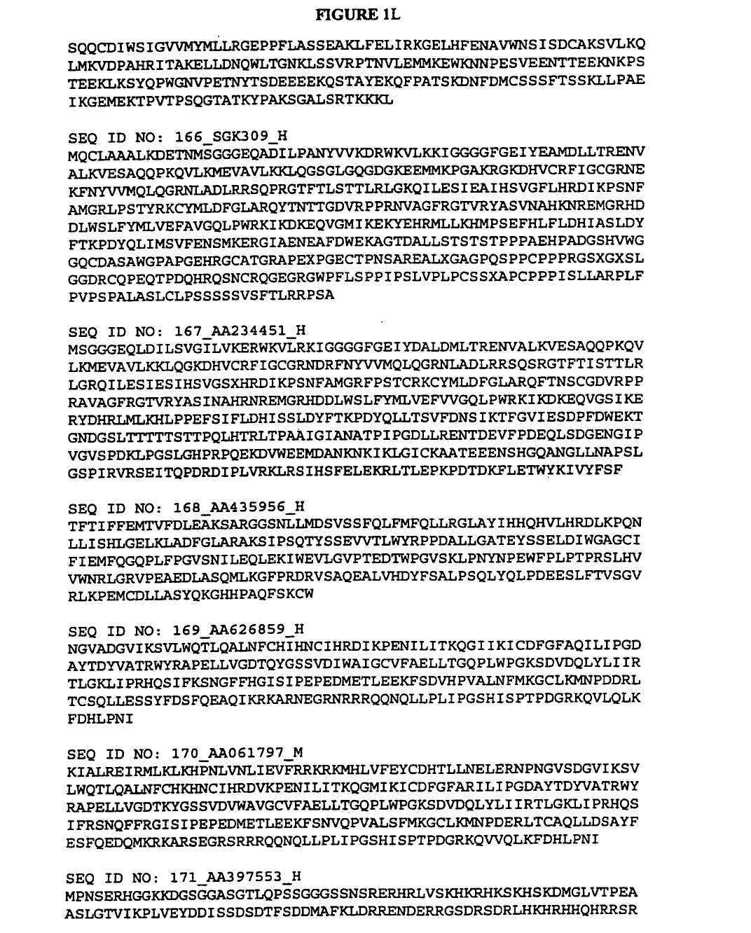

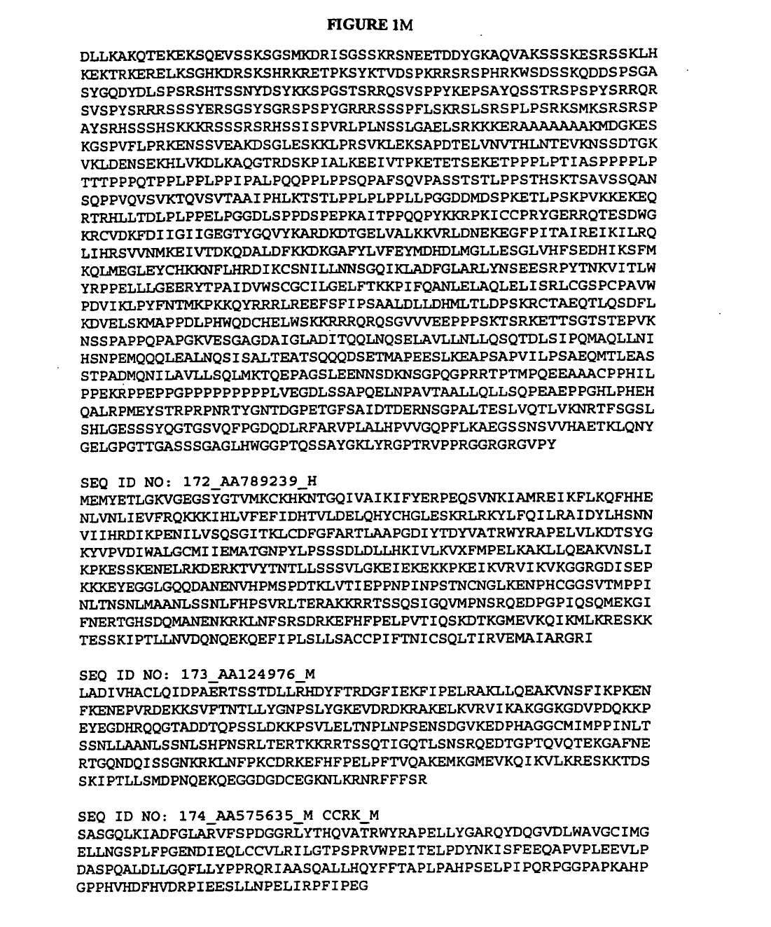

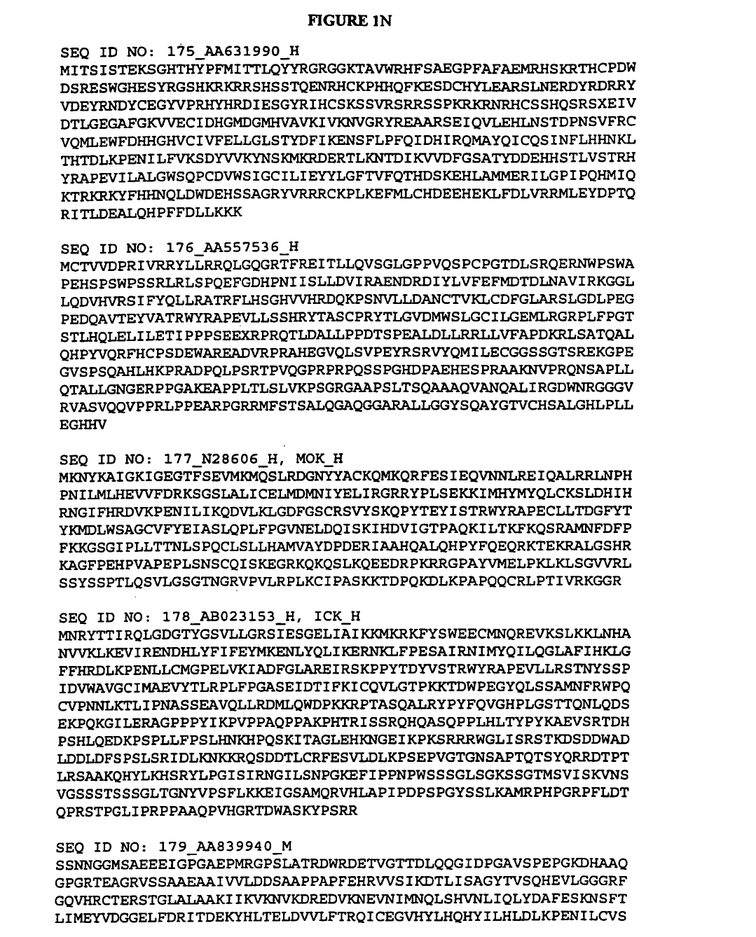

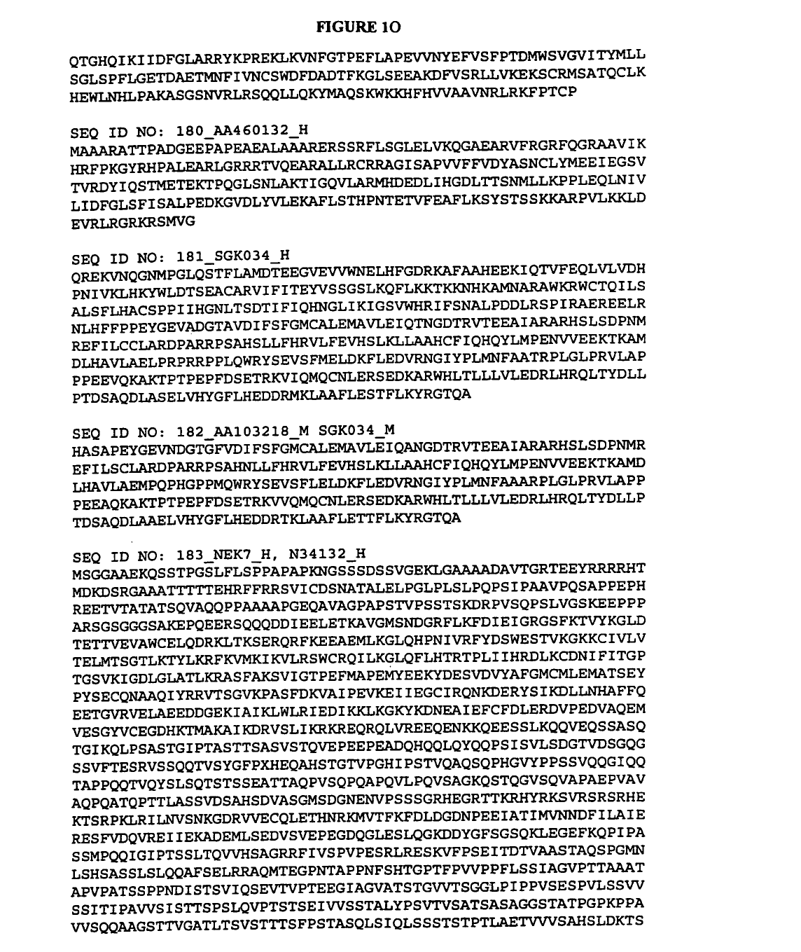

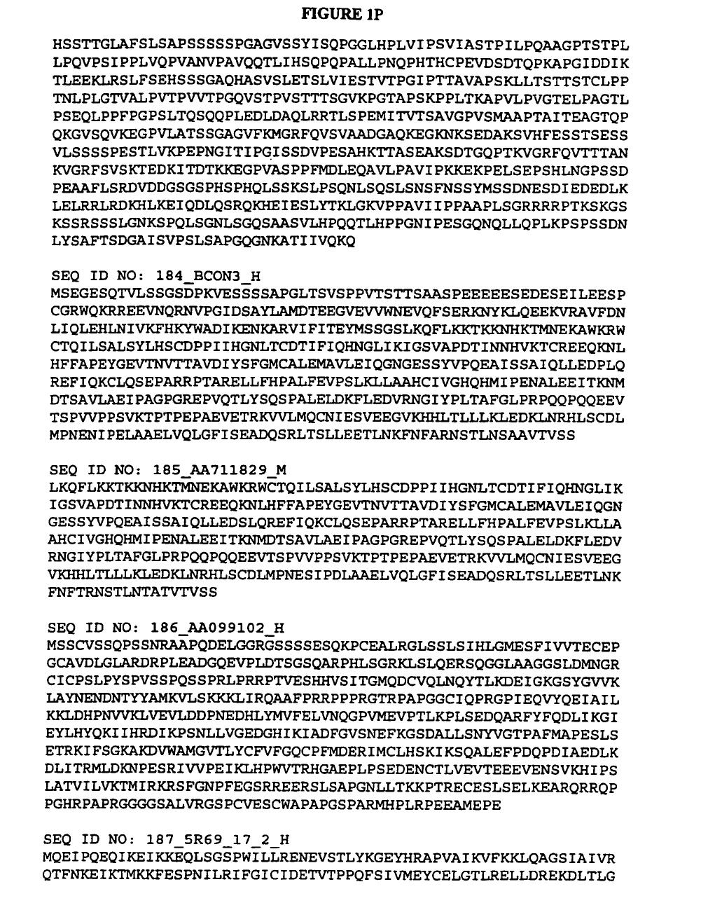

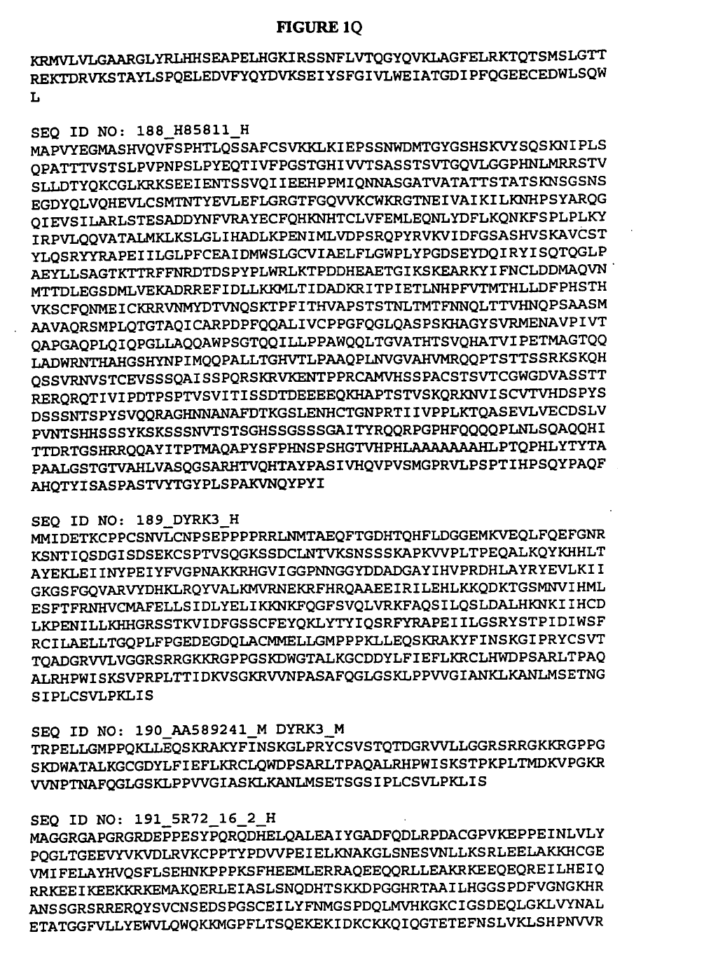

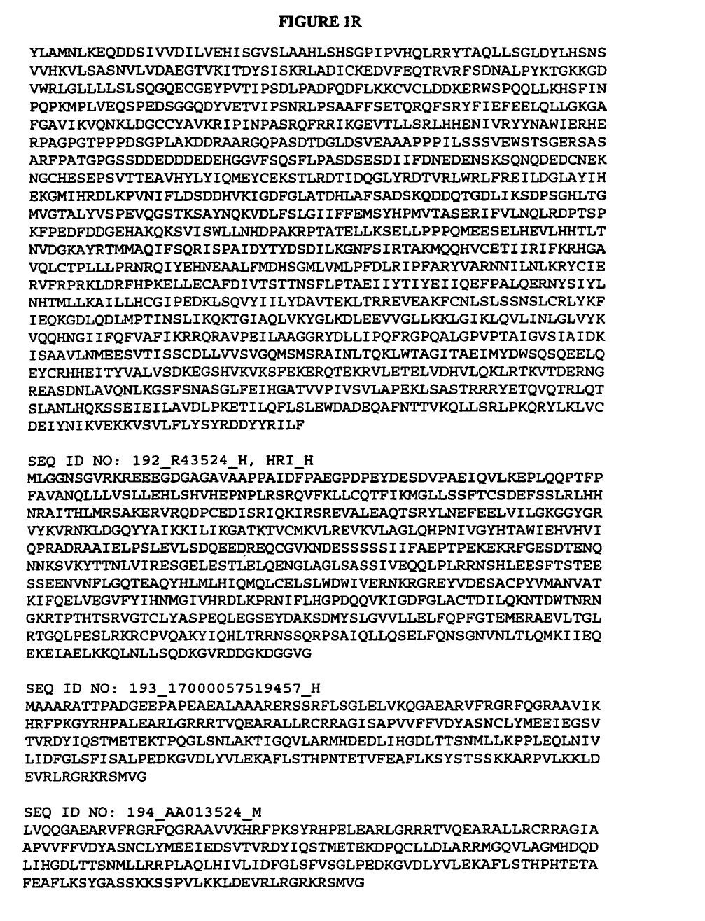

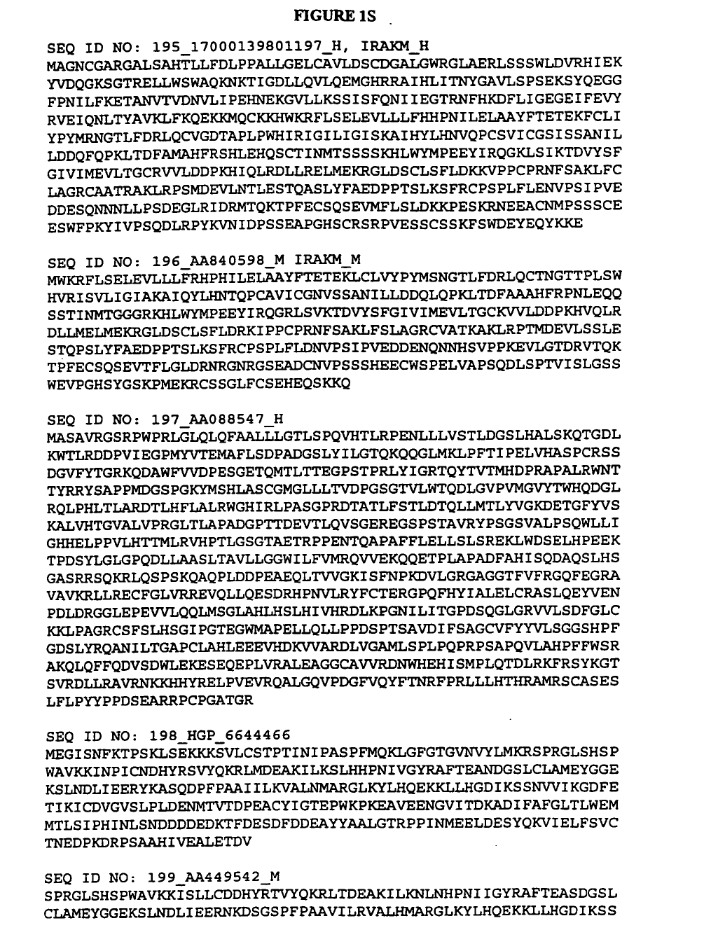

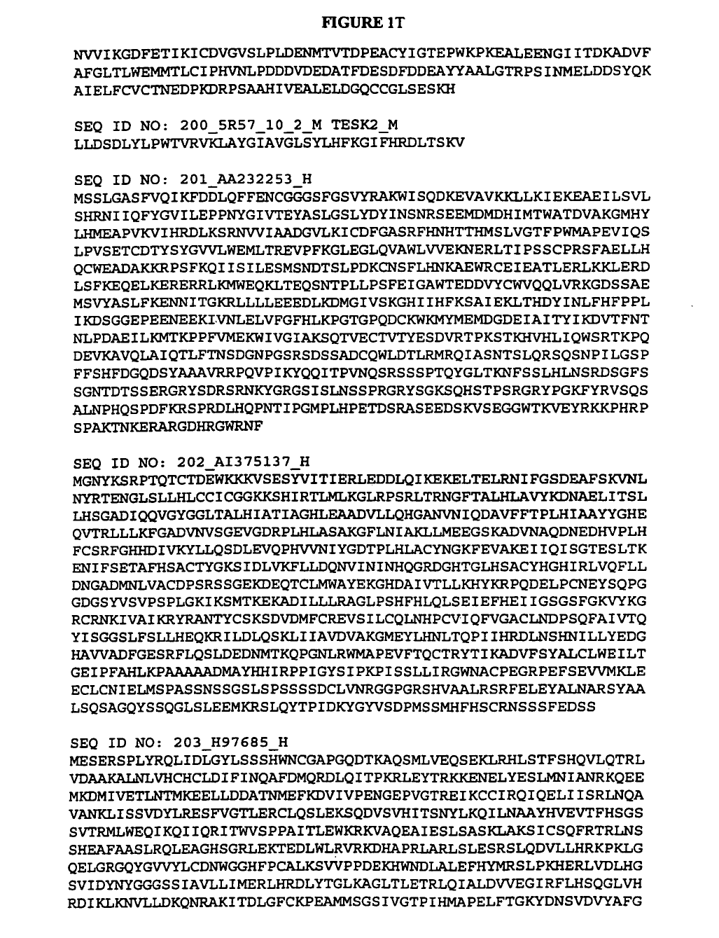

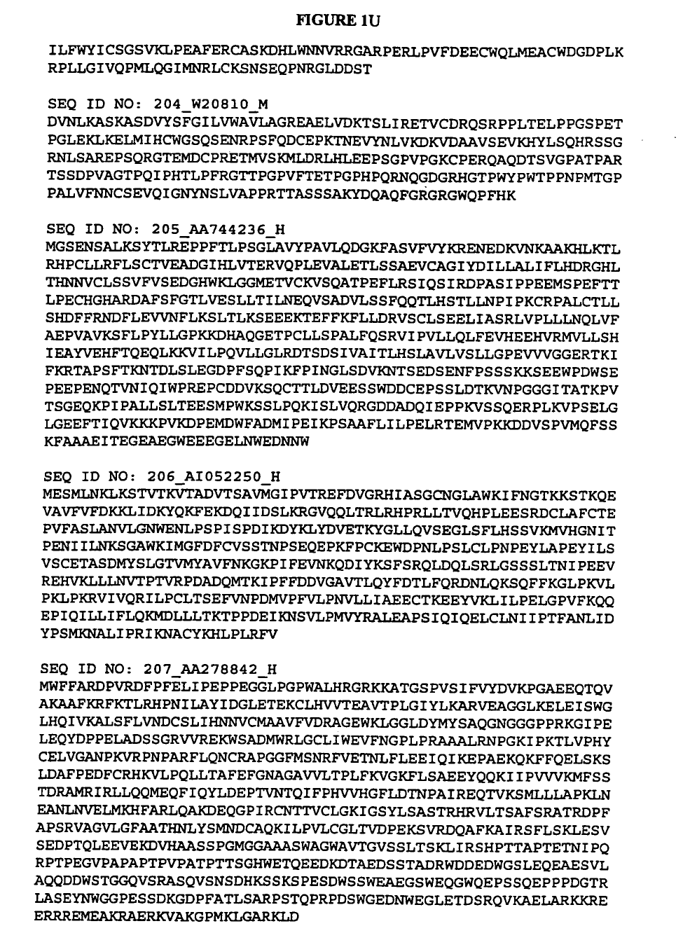

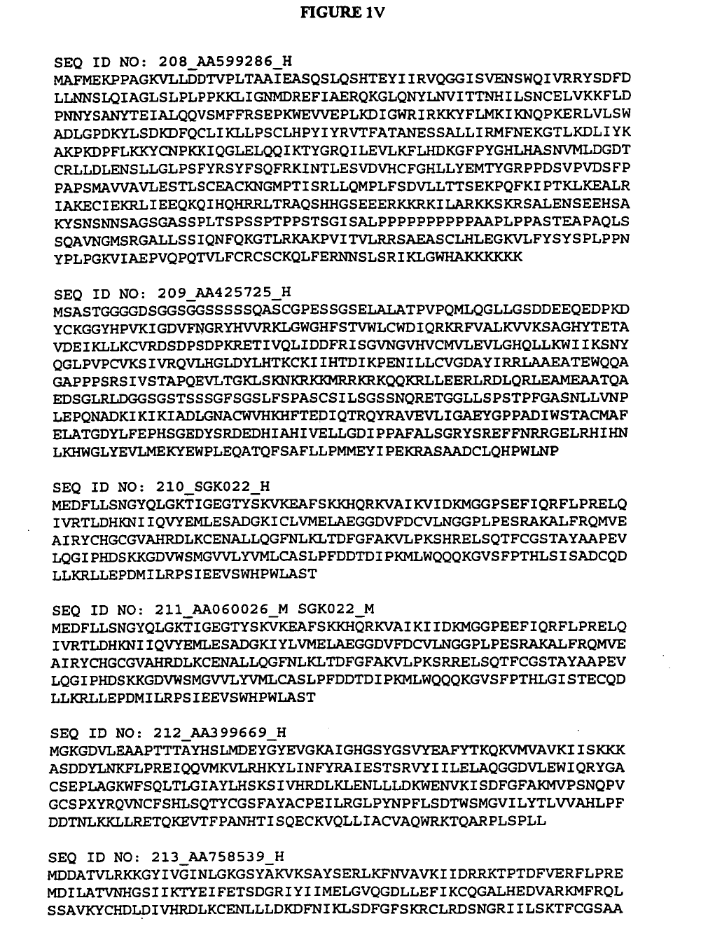

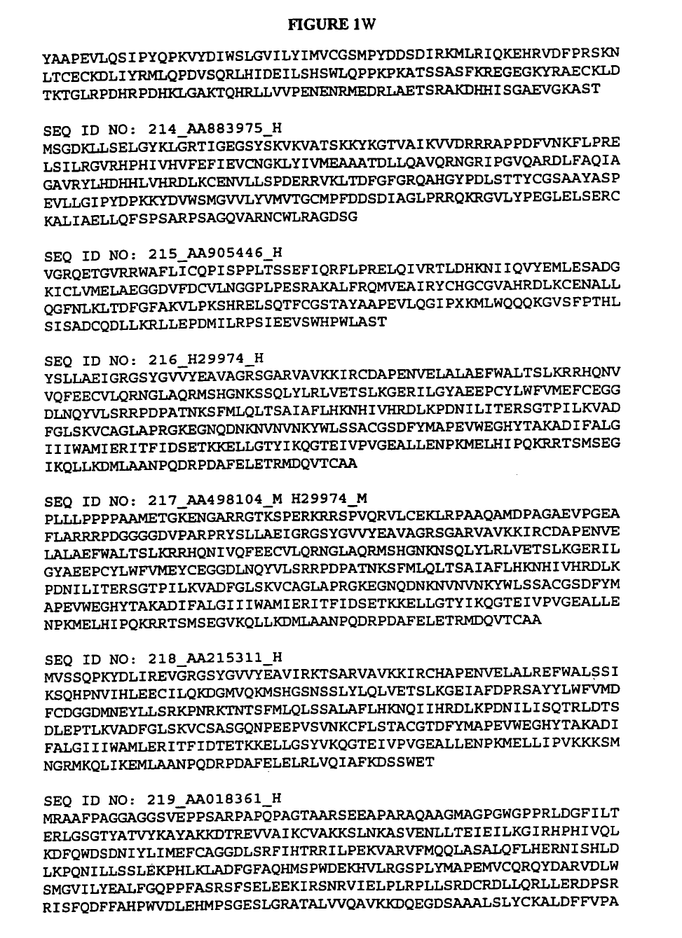

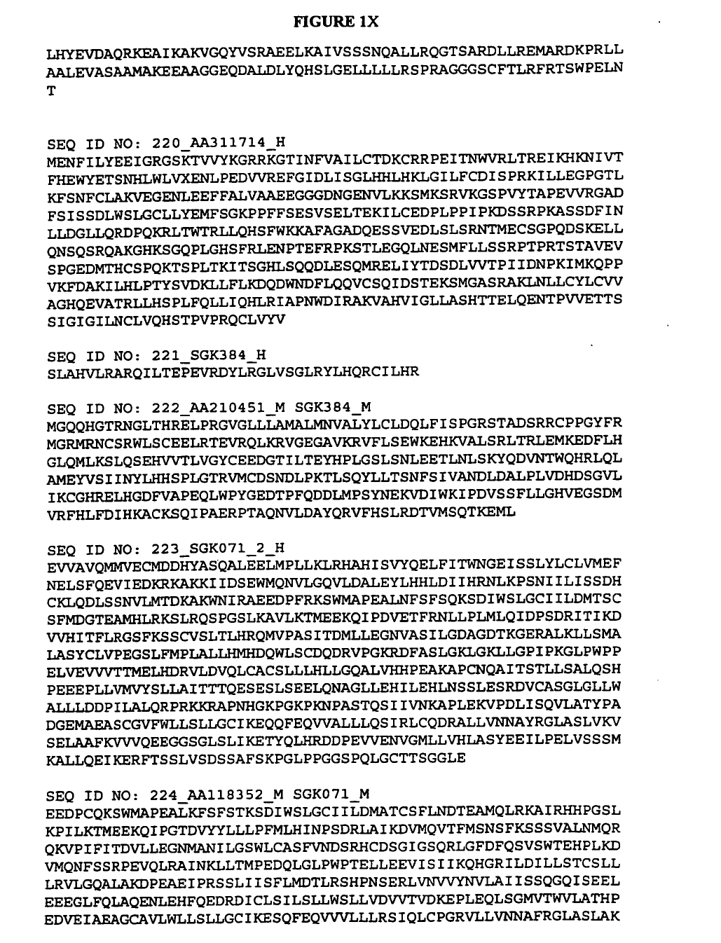

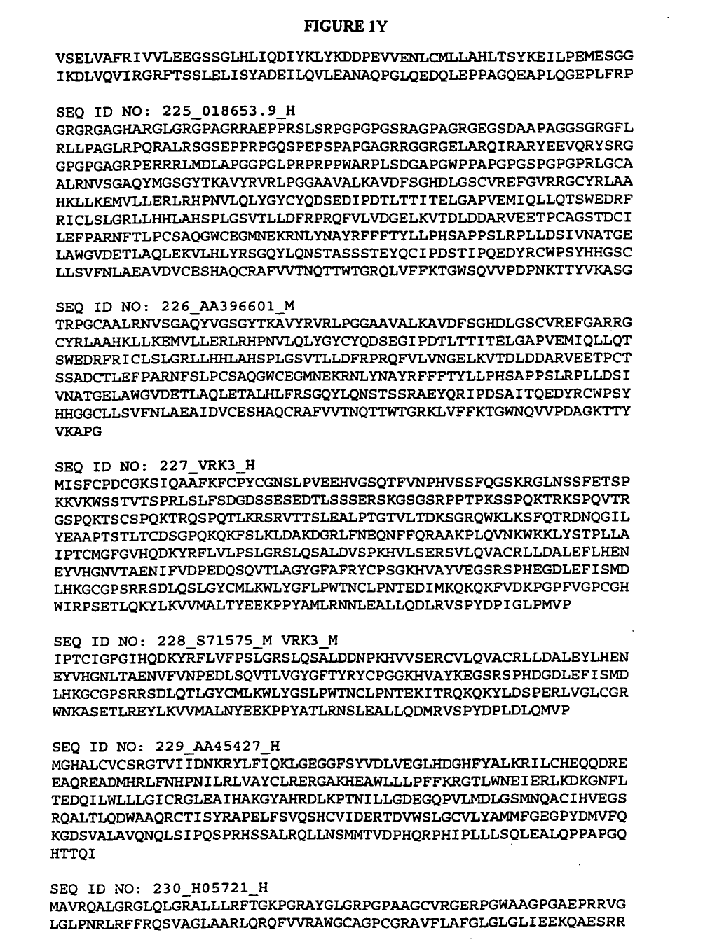

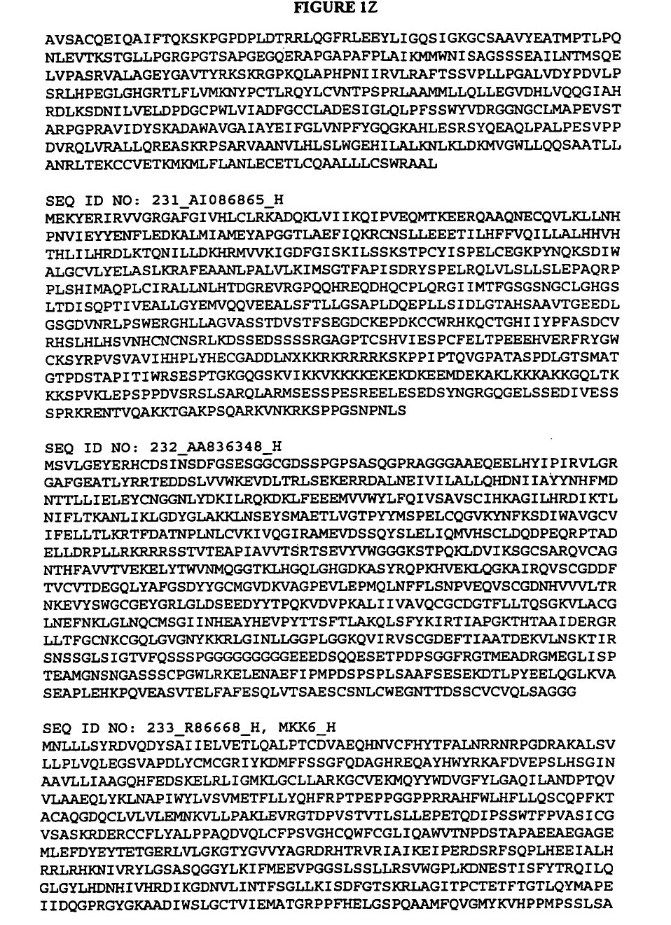

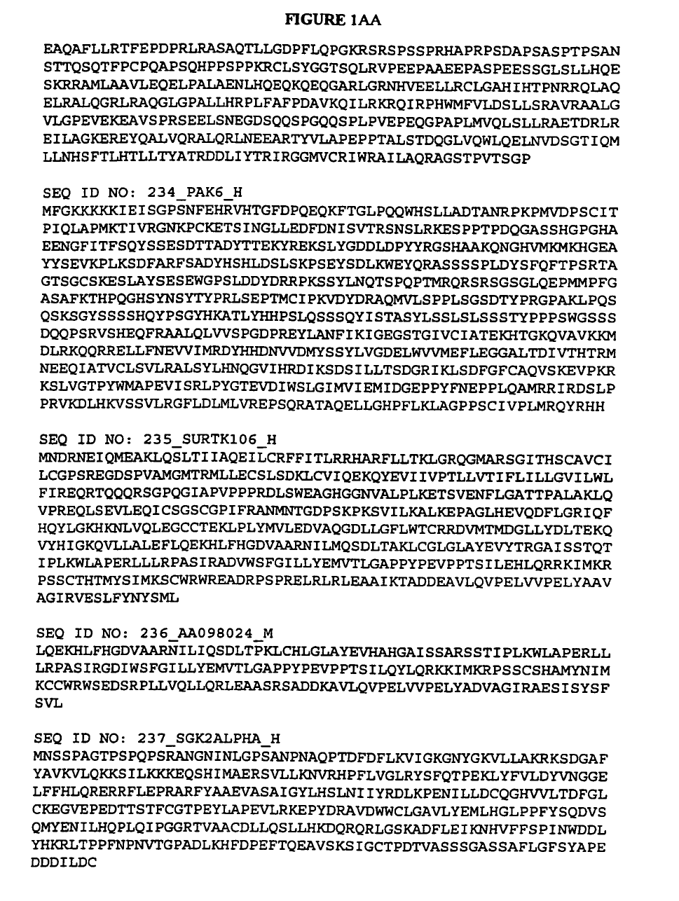

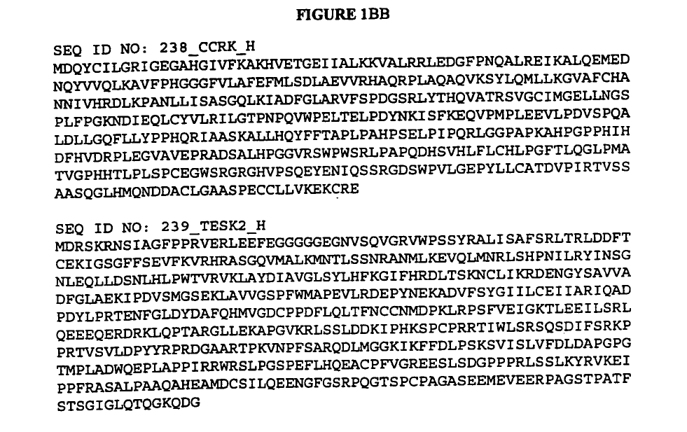

Further, the polypeptides of the invention include the full-length polypeptides that can be identified from the full-length or partial sequences encoded by SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 (FIG. 1). In addition, the polypeptides of the invention include the domains of these polypeptides, including, but not limited to, the N-terminal, kinase/catalytic, and C-terminal domains.

The characteristics of the protein kinase nucleic acid sequences of the invention are provided in Table 1. The protein kinases fall into 10 known groups: AGC, CAMK, CKI, CMGC, dsPK, EIFK, LIMK, MLK, STE and TY, In addition, there are a significant number of protein kinases that do not belong to any of the known groups, and therefore presumably define new protein kinase groups.

Additional characteristics are shown in, inter alia, the tables, namely Table 1, Table 2, Table 3 and Table 4, provided below.

V. Antibodies. Hybridomas, Methods of Use and Kits for Detection of Protein Kinases

The present invention relates to an antibody having binding affinity to a kinase of the invention. The polypeptide may have an amino acid sequence selected from the group consisting of those set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165. SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:188, SEQ ID NO:189, SEQ ID NO:190, SEQ ID NO:191, SEQ ID NO:199, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, or a functional derivative thereof, or at least 9 contiguous amino acids thereof (preferably, at least 20, 30, 35, or 40 or more contiguous amino acids thereof). Alternatively, the antibody may bind to a part of the polypeptide not provided in the sequences above, but that is present in the full-length sequence of the polypeptide and that is easily obtained using methods standard in the art. Further, the antibody may bind specifically to particular domains of one or more of the kinases of the invention, including, but not limited to, the N-terminal, kinase/catalytic, or C-terminal domains.

The present invention also relates to an antibody having specific binding affinity to a kinase or kinase domain of the invention. Such an antibody may be isolated by comparing its binding affinity to a kinase of the invention with its binding affinity to other polypeptides. Those that bind selectively to a kinase of the invention would be chosen for use in methods requiring a distinction between a kinase of the invention and other polypeptides. Such methods could include, but should not be limited to, the analysis of altered kinase expression in tissue containing other polypeptides.

The kinases of the present invention can be used in a variety of procedures and methods, such as for the generation of antibodies, for use in identifying pharmaceutical compositions, and for studying DNA/protein interaction.

The kinases of the present invention can be used to produce antibodies or hybridomas. One skilled in the art will recognize that if an antibody is desired, such a peptide could be generated as described herein and used as an immunogen. The antibodies of the present invention include monoclonal and polyclonal antibodies, as well fragments of these antibodies, and humanized forms. Humanized forms of the antibodies of the present invention may be generated using one of the procedures known in the art such as chimerization or CDR grafting.

The present invention also relates to a hybridoma that produces the above-described monoclonal antibody, or binding fragment thereof A hybridoma is an immortalized cell line that is capable of secreting a specific monoclonal antibody.

In general, techniques for preparing monoclonal antibodies and hybridomas are well known in the art (Campbell, “Monoclonal Antibody Technology: Laboratory Techniques in Biochemistry and Molecular Biology,” Elsevier Science Publishers, Amsterdam, The Netherlands, 1984; St. Groth et al., J. Immunol. Methods 35:1-21, 1980). Any animal (mouse, rabbit, and the like) which is known to produce antibodies can be immunized with the selected polypeptide. Methods for immunization are well known in the art. Such methods include subcutaneous or intraperitoneal injection of the polypeptide. One skilled in the art will recognize that the amount of polypeptide used for immunization will vary based on the animal that is immunized, the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal antibodies, spleen cells from the immunized animals are removed, fused with myeloma cells, such as SP2/0-Ag14 myeloma cells, and allowed to become monoclonal antibody producing hybridoma cells. Any one of a number of methods well known in the art can be used to identify the hybridoma cell that produces an antibody with the desired characteristics. These include screening the hybridomas with an ELISA assay, western blot analysis, or radioimmunoassay (Lutz et al., Exp. Cell Res. 175:109-124, 1988). Hybridomas secreting the desired antibodies are cloned and the class and subclass are determined using procedures known in the art (Campbell, “Monoclonal Antibody Technology: Laboratory Techniques in Biochemistry and Molecular Biology”, supra, 1984).

For polyclonal antibodies, antibody-containing antisera is isolated from the immunized animal and is screened for the presence of antibodies with the desired specificity using one of the above-described procedures. The above-described antibodies may be detectably labeled. Antibodies can be detectably labeled through the use of radioisotopes, affinity labels (such as biotin, avidin, and the like), enzymatic labels (such as horse radish peroxidase, alkaline phosphatase, and the like) fluorescent labels (such as FITC or rhodamine, and the like), paramagnetic atoms, and the like. Procedures for accomplishing such labeling are well-known in the art, for example, see Stemberger et al., J. Histochem. Cytochem. 18:315, 1970; Bayer et al., Meth. Enzym. 62:308-, 1979; Engval et al., Immunol. 109:129-, 1972; Goding, J. Immunol. Meth. 13:215-, 1976. The labeled antibodies of the present invention can be used for in vitro, in vivo, and in situ assays to identify cells or tissues that express a specific peptide.

The above-described antibodies may also be immobilized on a solid support. Examples of such solid supports include plastics such as polycarbonate, complex carbohydrates such as agarose and sepharose, acrylic resins and such as polyacrylamide and latex beads. Techniques for coupling antibodies to such solid supports are well known in the art (Weir et al., “Handbook of Experimental Immunology” 4th Ed., Blackwell Scientific Publications, Oxford, England, Chapter 10, 1986; Jacoby et al., Meth. Enzym. 34, Academic Press, N.Y., 1974). The immobilized antibodies of the present invention can be used for in vitro, in vivo, and in situ assays as well as in immunochromotography.

Furthermore, one skilled in the art can readily adapt currently available procedures, as well as the techniques, methods and kits disclosed herein with regard to antibodies, to generate peptides capable of binding to a specific peptide sequence in order to generate rationally designed antipeptide peptides (Hurby et al., “Application of Synthetic Peptides: Antisense Peptides”, In Synthetic Peptides, A User's Guide, W. H. Freeman, NY, pp. 289-307, 1992; Kaspczak et al., Biochemistry 28:9230-9238, 1989).

Anti-peptide peptides can be generated by replacing the basic amino acid residues found in the peptide sequences of the kinases of the invention with acidic residues, while maintaining hydrophobic and uncharged polar groups. For example, lysine, arginine, and/or histidine residues are replaced with aspartic acid or glutarnic acid and glutamic acid residues are replaced by lysine, arginine or histidine.

The present invention also encompasses a method of detecting a kinase polypeptide in a sample, comprising: (a) contacting the sample with an above-described antibody, under conditions such that immunocomplexes form, and (b) detecting the presence of said antibody bound to the polypeptide. In detail, the methods comprise incubating a test sample with one or more of the antibodies of the present invention and assaying whether the antibody binds to the test sample. Altered levels of a kinase of the invention in a sample as compared to normal levels may indicate disease.

Conditions for incubating an antibody with a test sample vary. Incubation conditions depend on the format employed in the assay, the detection methods employed, and the type and nature of the antibody used in the assay. One skilled in the art will recognize that any one of the commonly available immunological assay formats (such as radioimmunoassays, enzyme-linked immunosorbent assays, diffusion based Ouchterlony, or rocket immunofluorescent assays) can readily be adapted to employ the antibodies of the present invention. Examples of such assays can be found in Chard (“An Introduction to Radioimmunoassay and Related Techniques” Elsevier Science Publishers, Amsterdam, The Netherlands, 1986), Bullock et al. (“Techniques in Immunocytochemistry,” Academic Press, Orlando, Fla. Vol. 1, 1982; Vol. 2, 1983; Vol. 3, 1985), Tijssen (“Practice and Theory of Enzyme Immunoassays: Laboratory Techniques in Biochemistry and Molecular Biology,” Elsevier Science Publishers, Amsterdam, The Netherlands, 1985).

The immunological assay test samples of the present invention include cells, protein or membrane extracts of cells, or biological fluids such as blood, serum, plasma, or urine. The test samples used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is testable with the system utilized.

A kit contains all the necessary reagents to carry out the previously described methods of detection. The kit may comprise: (i) a first container means containing an above-described antibody, and (ii) second container means containing a conjugate comprising a binding partner of the antibody and a label. In another preferred embodiment, the kit further comprises one or more other containers comprising one or more of the following: wash reagents and reagents capable of detecting the presence of bound antibodies.

Examples of detection reagents include, but are not limited to, labeled secondary antibodies, or in the alternative, if the primary antibody is labeled, the chromophoric, enzymatic, or antibody binding reagents that are capable of reacting with the labeled antibody. The compartmentalized kit may be as described above for nucleic acid probe kits. One skilled in the art will readily recognize that the antibodies described in the present invention can readily be incorporated into one of the established kit formats that are well known in the art.

VI. Isolation of Compounds That Interact With Protein Kinases

The present invention also relates to a method of detecting a compound capable of binding to a protein kinase of the invention, comprising incubating the compound with a kinase of the invention and detecting the presence of the compound bound to the kinase. The compound may be present within a complex mixture, for example, serum, body fluid, or cell extracts.

The present invention also relates to a method of detecting an agonist or antagonist of kinase activity or kinase binding partner activity comprising incubating cells that produce a kinase of the invention in the presence of a compound and detecting changes in the level of kinase activity or kinase binding partner activity. The compounds thus identified would produce a change in activity indicative of the presence of the compound. The compound may be present within a complex mixture, for example, serum, body fluid, or cell extracts. Once the compound is identified it can be isolated using techniques well known in the art.

The present invention also encompasses a method of agonizing (stimulating) or antagonizing kinase associated activity in a mammal comprising administering to said mammal an agonist or antagonist to a kinase of the invention in an amount sufficient to effect said agonism or antagonism. A method of treating diseases in a mammal with an agonist or antagonist of kinase activity comprising administering the agonist or antagonist to a mammal in an amount sufficient to agonize or antagonize kinase associated functions is also encompassed in the present application.

In an effort to discover novel treatments for diseases, biomedical researchers and chemists have designed, synthesized, and tested molecules that inhibit the function of protein kinases. Some small organic molecules form a class of compounds that modulate the function of protein kinases. Examples of molecules that have been reported to inhibit the function of protein kinases include, but are not limited to, bis monocyclic, bicyclic or heterocyclic aryl compounds (PCT WO 92120642, published Nov. 26, 1992 by Maguire et al.), vinylene-azaindole derivatives (PCT WO 94/14808, published Jul. 7, 1994 by Ballinari et al.), 1-cyclopropyl-4-pyridyl-quinolones (U.S. Pat. No. 5,330,992), styryl compounds (U.S. Pat. No. 5,217,999), styryl-substituted pyridyl compounds (U.S. Pat. No. 5,302,606), certain quinazoline derivatives (EP Application No. 0 566 266 A1), seleoindoles and selenides (PCT WO 94/03427, published Feb. 17, 1994 by Denny et al.), tricyclic polyhydroxylic compounds (PCT WO 92/21660, published Dec. 10, 1992 by Dow), and benzylphosphonic acid compounds (PCT WO 91/15495, published Oct. 17, 1991 by Dow et al).

Compounds that can traverse cell membranes and are resistant to acid hydrolysis are potentially advantageous as therapeutics as they can become highly bioavailable after being administered orally to patients. However, many of these protein kinase inhibitors only weakly inhibit the function of protein kinases. In addition, many inhibit a variety of protein kinases and will cause multiple side-effects as therapeutics for diseases.

Some indolinone compounds, however, form classes of acid resistant and membrane permeable organic molecules. WO 96/22976 (published Aug. 1, 1996 by Ballinari et al.) describes hydrosoluble indolinone compounds that harbor tetralin, naphthalene, quinoline, and indole substituents fused to the oxindole ring. These bicyclic substituents are in turn substituted with polar moieties including hydroxylated alkyl, phosphate, and ether moieties. U.S. patent application Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187) and Ser. No. 08/485,323, filed Jun. 7, 1995, entitled “Benzylidene-Z-Indoline Compounds for the Treatment of Disease” by Tang et al. (Lyon & Lyon Docket No. 223/298) and International Patent Publication WO 96/22976, published Aug. 1, 1996 by Ballinari et al., all of which are incorporated herein by reference in their entirety, including any drawings, describe indolinone chemical libraries of indolinone compounds harboring other bicyclic moieties as well as monocyclic moieties fused to the oxindole ring. Applications Ser. No. 08/702,232, filed Aug. 23, 1996, entitled “Indolinone Combinatorial Libraries and Related Products and Methods for the Treatment of Disease” by Tang et al. (Lyon & Lyon Docket No. 221/187), Ser. No. 08/485,323, filed Jun. 7, 1995, entitled “Benzylidene-Z-Indoline Compounds for the Treatment of Disease” by Tang et al. (Lyon & Lyon Docket No. 223/298), and WO 96/22976, published Aug. 1, 1996 by Ballinari et al teach methods of indolinone synthesis, methods of testing the biological activity of indolinone compounds in cells, and inhibition patterns of indolinone derivatives.

Other examples of substances capable of modulating kinase activity include, but are not limited to, tyrphostins, quinazolines, quinoxolines, and quinolines. The quinazolines, tyrphostins, quinolines, and quinoxolines referred to above include well known compounds such as those described in the literature. For example, representative publications describing quinazolines include Barker et al., EPO Publication No. 0 520 722 A1; Jones et al., U.S. Pat. No.4,447,608; Kabbe et al., U.S. Pat. No. 4,757,072; Kaul and Vougioukas, U.S. Pat. No. 5, 316,553; Kreighbaum and Comer, U.S. Pat. No. 4,343,940; Pegg and Wardleworth, EPO Publication No. 0 562 734 A1; Barker et al., Proc. of Am. Assoc. for Cancer Research 32:327 (1991); Bertino, J. R, Cancer Research 3:293-304 (1979); Bertino, J. R., Cancer Research 9(2 part 1):293-304 (1979); Curtin te al., Br. J. Cancer 53:361-368 (1986); Fernandes et al., Cancer Research 43:1117-1123 (1983); Ferris et al. J. Org. Chem. 44(2):173-178; Fry et al., Science 265:1093-1095 (1994); Jackman et al., Cancer Research 51:5579-5586 (1981); Jones et al. J. Med. Chem. 29(6):1114-1118; Lee and Skibo, Biochemistry 26(23):7355-7362 (1987); Lemus et al., J. Org. Chem. 54:3511-3518 (1989); Ley and Seng, Synthesis 1975:415-522 (1975); Maxwell et al., Magnetic Resonance in Medicine 17:189-196 (1991); Mini et al., Cancer Research 45:325-330 (1985); Phillips and Castle, J. Heterocyclic Chem. 17(19):1489-1596 (1980); Reece et al., Cancer Research 47(11):2996-2999 (1977); Sculier et al., Cancer Immunol. and Immunother. 23:A65 (1986); Sikora et al., Cancer Letters 23:289-295 (1984); Sikora et al., Analytical Biochem. 172:344-355 (1988); all of which are incorporated herein by reference in their entirety, including any drawings.

Quinoxaline is described in Kaul and Vougioukas, U.S. Pat. No. 5,316,553, incorporated herein by reference in its entirety, including any drawings.

Quinolines are described in Dolle et al., J. Med. Chem. 37:2627-2629 (1994); MaGuire, J. Med. Chem. 37:2129-2131 (1994); Burke et al., J. Med. Chem. 36:425-432 (1993); and Burke et al. BioOrganic Med. Chem. Letters 2:1771-1774 (1992), all of which are incorporated by reference in their entirety, including any drawings.

Tyrphostins are described in Allen et al., Clin. Exp. Immunol. 91:141-156 (1993); Anafi et al., Blood 82:12:3524-3529 (1993); Baker et al., J. Cell Sci. 102:543-555 (1992); Bilder et al., Amer. Physiol. Soc. pp. 6363-6143:C721-C730 (1991); Brunton et al., Proceedings of Amer. Assoc. Cancer Rsch. 33:558 (1992); Bryckaert et al., Experimental Cell Research 199:255-261 (1992); Dong et al., J. Leukocyte Biology 53:53-60 (1993); Dong et al, J. Immunol. 151(5):2717-2724 (1993); Gazit et al., J. Med. Chem. 32:2344-2352 (1989); Gazit et al., “J. Med. Chem. 36:3556-3564 (1993); Kaur et al., Anti-Cancer Drugs 5:213-222 (1994); Kaur et al., King et al., Biochem. J. 275:413-418 (1991); Kuo et al., Cancer Letters 74:197-202 (1993); Levitzki, A., The FASEB J. 6:3275-3282 (1992); Lyall et al., J. Biol. Chem. 264:14503-14509 (1989); Peterson et al., The Prostate 22:335-345 (1993); Pillemer et al., Int. J. Cancer 50:80-85 (1992); Posner et al., Molecular Pharmacology 45:673-683 (1993); Rendu et al., Biol. Pharmacology 44(5):881-888 (1992); Sauro and Thomas, Life Sciences 53:371-376 (1993); Sauro and Thomas, J. Pharm. and Experimental Therapeutics 267(3):119-1125 (1993); Wolbring et al., J. Biol. Chem. 269(36):22470-22472 (1994); and Yoneda et al., Cancer Research 51:4430-4435 (1991); all of which are incorporated herein by reference in their entirety, including any drawings.

Other compounds that could be used as modulators include oxindolinones such as those described in U.S. patent application Ser. No. 08/702,232 filed Aug. 23, 1996, incorporated herein by reference in its entirety, including any drawings.

VII. Biological Significance, Applications and Clinical Relevance of Novel Protein Kinases

For each protein kinase in this application, we provide a classification of the protein class and family to which it belongs, a summary of non-cataltyic protein motifs, a profile of its expression in several hundred tissue and cell sources, and a chromosomal location. This information can be used to suggest potential function, regulation or therapeutic utility for each of the proteins.

The kinase classification and protein domains often reflect pathways, cellular roles, or mechanisms of up- or down-stream regulation. Also disease-relevant genes often occur in families of related genes. For example if one member of a kinase family functions as an oncogene, a tumor suppressor, or has been found to be disrupted in an immune, neurologic, cardiovascular, or metabolic disorder, frequently other family members may play a related role.

The expression analysis organizes kinases into groups that are transcriptionally upregulated in tumors and those that are more restricted to specific tumor types such as melanoma or prostate. This analysis also identifies genes that are regulated in a cell cycle dependent manner, and are therefore likely to be involved in maintaining cell cycle checkpoints, entry, progression, or exit from mitosis, oversee DNA repair, or are involved in cell proliferation and genome stability. Expression data also can identify genes expressed in endothelial sources or other tissues that suggest a role in angiogenesis, thereby implicating them as targets for control of diseases that have an angiogenic component, such as cancer, endometriosis, retinopathy and macular degeneration, and various ischemic or vascular pathologies. A proteins' role in cell survival can also be suggested based on restricted expression in cells subjected to external stress such as oxidative damage, hypoxia, drugs such as cisplatinum, or irradiation. Metastases-associated genes can be implicated when expression is restricted to invading regions of a tumor, or is only seen in local or distant metastases compared to the primary tumor, or when a gene is upregulated during cell culture models of invasion, migration, or motility.

Chromosomal location can identify candidate targets for a tumor amplicon or a tumor-suppressor locus. Summaries of prevelant tumor amplicons are available in the literature, and can identify tumor types to experimentally be confirmed to contain amplified copies of a kinase gene which localizes to an adjacent region.

Based on these criteria several kinases immediately stand out as being of potential therapeutic relevance. The protein kinases can be divided into the following disease-relevant categories (nucleotide Seq ID #s in parentheses):

Tumor associated: Mok (SEQ ID NO:NO:57), EPK2, AA316804 (SEQ ID NO:11), AA435956 (SEQ ID NO:NO:48), AA278842 (SEQ ID NO:88), AA599286 (SEQ ID NO:89), AA826850 (SEQ ID NO:3), HRI (SEQ ID NO:73), MLK4 AA232253 (SEQ ID NO:82), AA883975 SGK 235 (SEQ ID NO:95), AA311714 (SEQ ID NO:101), MPSK1 (SEQ ID NO:110), R19609 (Seq ID111), AA383293 (SEQ ID NO:26).

Prostate-specific: AA234451 (SEQ ID NO:47), TSK4 (SEQ ID NO:93), RIP4 (SEQ ID NO:84), KIAA0965 (SEQ ID NO:8).

Oncogenic or proliferation associated: KIAA0781 (SEQ ID NO:38), AA789239 (SEQ ID NO:52), CCRK (SEQ ID NO:54), CLK4 (SEQ ID NO:55), H85389 (SEQ ID NO:97).

Neuronal restricted: CAMKKB (SEQ ID NO:66)

Hematopoietic expressed: PTK9L (SEQ ID NO:22), DRAK2 (SEQ ID NO:29), AI025291 (SEQ ID NO:94)

Angiogenic or endothelial expressed: DRAK1 (SEQ ID NO:31), MAK-V (SEQ ID NO:40), TRAD (SEQ ID NO:44), MOK (SEQ ID NO:57), AA08847 (SEQ ID NO:78), HGP_{—}66444466 (SEQ ID NO:79), RSK4 (SEQ ID NO:16).

Cell cycle regulated: AA454060 (SEQ ID NO:45), KIAA0999 (Mitotic—SEQ ID NO:32), AA579641 (Mitotic—SEQ ID NO:60), AA305176 (Mitotic—SEQ ID NO:6), AA018361 (S1 phase—SEQ ID NO:100).

VIII. Transgenic Animals.

A variety of methods are available for the production of transgenic animals associated with this invention. DNA can be injected into the pronucleus of a fertilized egg before fusion of the male and female pronuclei, or injected into the nucleus of an embryonic cell (e.g., the nucleus of a two-cell embryo) following the initiation of cell division (Brinster et al., Proc. Nat. Acad. Sci. USA 82: 4438-4442, 1985) Embryos can be infected with viruses, especially retroviruses, modified to carry inorganic-ion receptor nucleotide sequences of the invention.

Pluripotent stem cells derived from the inner cell mass of the embryo and stabilized in culture can be manipulated in culture to incorporate nucleotide sequences of the invention. A transgenic animal can be produced from such cells through implantation into a blastocyst that is implanted into a foster mother and allowed to come to term. Animals suitable for tansgenic experiments can be obtained from standard commercial sources such as Charles River (Wilmington, Mass.), Taconic (Germantown, N.Y.), Harlan Sprague Dawley (Indianapolis, Ind.), etc.

The procedures for manipulation of the rodent embryo and for microinjection of DNA into the pronucleus of the zygote are well known to those of ordinary skill in the art (Hogan et al., supra). Microinjection procedures for fish, amphibian eggs and birds are detailed in Houdebine and Chourrout (Experientia 47: 897-905, 1991). Other procedures for introduction of DNA into tissues of animals are described in U.S. Pat. No. 4,945,050 (Sanford el al., Jul. 30, 1990).

By way of example only, to prepare a transgenic mouse, female mice are induced to superovulate. Females are placed with males, and the mated females are sacrificed by CO₂ asphyxiation or cervical dislocation and embryos are recovered from excised oviducts. Surrounding cumulus cells are removed. Pronuclear embryos are then washed and stored until the time of injection. Randomly cycling adult female mice are paired with vasectomized males. Recipient females are mated at the same time as donor females. Embryos then are transferred surgically. The procedure for generating transgenic rats is similar to that of mice (Hammer et al., Cell 63:1099-1112, 1990).

Methods for the culturing of embryonic stem (ES) cells and the subsequent production of transgenic animals by the introduction of DNA into ES cells using methods such as electroporation, calcium phosphate/DNA precipitation and direct injection also are well known to those of ordinary skill in the art (Teratocarcinomas and Embryonic Stem Cells, A Practical Approach, E.J. Robertson, ed., JRL Press, 1987).

In cases involving random gene integration, a clone containing the sequence(s) of the invention is co-transfected with a gene encoding resistance. Alternatively, the gene encoding neomycin resistance is physically linked to the sequence(s) of the invention. Transfection and isolation of desired clones are carried out by any one of several methods well known to those of ordinary skill in the art (E. J. Robertson, supra).

DNA molecules introduced into ES cells can also be integrated into the chromosome through the process of homologous recombination (Capecchi, Science 244: 1288-1292, 1989). Methods for positive selection of the recombination event (i.e., neo resistance) and dual positive-negative selection (i.e., neo resistance and gancyclovir resistance) and the subsequent identification of the desired clones by PCR have been described by Capecchi, supra and Joyner et al. (Nature 338: 153-156, 1989), the teachings of which are incorporated herein in their entirety including any drawings. The final phase of the procedure is to inject targeted ES cells into blastocysts and to transfer the blastocysts into pseudopregnant females. The resulting chimeric animals are bred and the offspring are analyzed by Southern blotting to identify individuals that carry the transgene. Procedures for the production of non-rodent mammals and other animals have been discussed by others (Houdebine and Chourrout, supra; Pursel et al., Science 244:1281 -1288, 1989; and Simms et al., Bio/Technology 6:179-183, 1988).

Thus, the invention provides transgenic, nonhuman mammals containing a transgene encoding a kinase of the invention or a gene effecting the expression of the kinase. Such transgenic nonhuman mammals are particularly useful as an in vivo test system for studying the effects of introduction of a kinase, or regulating the expression of a kinase (ie., through the introduction of additional genes, antisense nucleic acids, or ribozymes).

A “Transgenic animal” is an animal having cells that contain DNA which has been artificially inserted into a cell, which DNA becomes part of the genome of the animal which develops from that cell. Preferred transgenic animals are primates, mice, rats, cows, pigs, horses, goats, sheep, dogs and cats. The transgenic DNA may encode human STE20-related kinases. Native expression in an animal may be reduced by providing an amount of anti-sense RNA or DNA effective to reduce expression of the receptor.

IX. Gene Therapy

Protein kinases of the invention, or their genetic sequences will also be useful in gene therapy (reviewed in Miller, Nature 357:455-460, 1992). Miller states that advances have resulted in practical approaches to human gene therapy that have demonstrated positive initial results. The basic science of gene therapy is described in Mulligan (Science 260:926-931, 1993).

In one preferred embodiment, an expression vector containing protein kinase coding sequence is inserted into cells, the cells are grown in vitro, and then are infused in large numbers into patients. In another preferred embodiment, a DNA segment containing a promoter of choice (for example a strong promoter) is transferred into cells containing an endogenous gene encoding kinases of the invention in such a manner that the promoter segment enhances expression of the endogenous kinase gene (for example, the promoter segment is transferred to the cell such that it becomes directly linked to the endogenous kinase gene).

The gene therapy may involve the use of an adenovirus containing kinase cDNA targeted to a tumor, systemic kinase increase by implantation of engineered cells, injection with kinase-encoding virus, or injection of naked kinase DNA into appropriate tissues.

Target cell populations may be modified by introducing altered forms of one or more components of the protein complexes in order to modulate the activity of such complexes. For example, by reducing or inhibiting a complex component activity within target cells, an abnormal signal transduction event(s) leading to a condition may be decreased, inhibited, or reversed. Deletion or missense mutants of a component, that retain the ability to interact with other components of the protein complexes but cannot function in signal transduction may be used to inhibit an abnormal, deleterious signal transduction event.

Expression vectors derived from viruses such as retroviruses, vaccinia virus, adenovirus, adeno-associated virus, herpes viruses, several RNA viruses, or bovine papilloma virus, may be used for delivery of nucleotide sequences (e.g., cDNA) encoding recombinant kinase of the invention protein into the targeted cell population (e.g. tumor cells). Methods which are well known to those skilled in the art can be used to construct recombinant viral vectors containing coding sequences (Maniatis et al., Molecular Cloning: A Laboratory Manual, Cold Spring Harbor Laboratory, N.Y., 1989; Ausubel et al., Current Protocols in Molecular Biology, Greene Publishing Associates and Wiley Interscience, N.Y., 1989). Alternatively, recombinant nucleic acid molecules encoding protein sequences can be used as naked DNA or in a reconstituted system e.g., liposomes or other lipid systems for delivery to target cells (e.g., Feigner et al., Nature 337:387-8, 1989). Several other methods for the direct transfer of plasmid DNA into cells exist for use in human gene therapy and involve targeting the DNA to receptors on cells by complexing the plasmid DNA to proteins (Miller, supra).

In its simplest form, gene transfer can be performed by simply injecting minute amounts of DNA into the nucleus of a cell, through a process of microinjection (Capecchi, Cell 22:479-88, 1980). Once recombinant genes are introduced into a cell, they can be recognized by the cell's normal mechanisms for transcription and translation, and a gene product will be expressed. Other methods have also been attempted for introducing DNA into larger numbers of cells. These methods include: transfection, wherein DNA is precipitated with CaPO₄ and taken into cells by pinocytosis (Chen et al., Mol. Cell Biol. 7:2745-52, 1987); electroporation, wherein cells are exposed to large voltage pulses to introduce holes into the membrane (Chu et al., Nucleic Acids Res. 15:1311-26, 1987); lipofection/liposome fusion, wherein DNA is packaged into lipophilic vesicles which fuse with a target cell (Felgner et al., Proc. Natl. Acad. Sci. USA. 84:7413-7417, 1987); and particle bombardment using DNA bound to small projectiles (Yang et al., Proc. Natl. Acad. Sci. 87:9568-9572, 1990). Another method for introducing DNA into cells is to couple the DNA to chemically modified proteins.

It has also been shown that adenovirus proteins are capable of destabilizing endosomes and enhancing the uptake of DNA into cells. The admixture of adenovirus to solutions containing DNA complexes, or the binding of DNA to polylysine covalently attached to adenovirus using protein crosslinking agents substantially improves the uptake and expression of the recombinant gene (Curiel et al., Am. J. Respir. Cell. Mol. Biol., 6:247-52, 1992).

As used herein “gene transfer” means the process of introducing a foreign nucleic acid molecule into a cell. Gene transfer is commonly performed to enable the expression of a particular product encoded by the gene. The product may include a protein, polypeptide, anti-sense DNA or RNA, or enzymatically active RNA. Gene transfer can be performed in cultured cells or by direct administration into animals. Generally gene transfer involves the process of nucleic acid contact with a target cell by non-specific or receptor mediated interactions, uptake of nucleic acid into the cell through the membrane or by endocytosis, and release of nucleic acid into the cytoplasm from the plasma membrane or endosome. Expression may require, in addition, movement of the nucleic acid into the nucleus of the cell and binding to appropriate nuclear factors for transcription.

As used herein “gene therapy” is a form of gene transfer and is included within the definition of gene transfer as used herein and specifically refers to gene transfer to express a therapeutic product from a cell in vivo or in vitro. Gene transfer can be performed ex vivo on cells which are then transplanted into a patient, or can be performed by direct administration of the nucleic acid or nucleic acid-protein complex into the patient.

In another preferred embodiment, a vector having nucleic acid sequences encoding a protein Iinase polypeptide of the invention is provided in which the nucleic acid sequence is expressed only in specific tissue. Methods of achieving tissue-specific gene expression are set forth in International Publication No. WO 93/09236, filed Nov. 3, 1992 and published May 13, 1993.

In all of the preceding vectors set forth above, a further aspect of the invention is that the nucleic acid sequence contained in the vector may include additions, deletions or modifications to some or all of the sequence of the nucleic acid, as defined above.

In another preferred embodiment, a method of gene replacement is set forth. “Gene replacement” as used herein means supplying a nucleic acid sequence which is capable of being expressed in vivo in an animal and thereby providing or augmenting the function of an endogenous gene that is missing or defective in the animal.

X. Administration of Substances

Methods of determining the dosages of compounds to be administered to a patient and modes of administering compounds to an organism are disclosed in U.S. application Ser. No. 08/702,282, filed Aug. 23, 1996 and International patent publication number WO 96/22976, published Aug. 1, 1996, both of which are incorporated herein by reference in their entirety, including any drawings, figures, or tables. Those skilled in the art will appreciate that such descriptions are applicable to the present invention and can be easily adapted to it.

The proper dosage depends on various factors such as the type of disease being treated, the particular composition being used, and the size and physiological condition of the patient. Therapeutically effective doses for the compounds described herein can be estimated initially from cell culture and animal models. For example, a dose can be formulated in animal models to achieve a circulating concentration range that initially takes into account the IC₅₀ as determined in cell culture assays. The animal model data can be used to more accurately determine useful doses in humans.

Plasma half-life and biodistribution of the drug and metabolites in the plasma, tumors, and major organs can be also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan, and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition, and the percent of lymphocytes versus polyrnorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, Journal of American Veterinary Medical Assoc., 202:229-249, 1993). Representative animals from each treatment group can then be examined by gross necropsy for immediate evidence of metastasis, unusual illness, or toxicity. Gross abnormalities in tissue are noted, and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

### EXAMPLES

The examples below are not limiting and are merely representative of various aspects and features of the present invention. The examples below demonstrate the isolation and characterization of the protein kinases of the invention.

### Example 1

### Isolation of cDNA Clones Encoding Novel Mammalian Protein Kinases Materials and Methods Identification from cDNA Databases and Isolation of Clones Encoding Novel Protein Kinases

Novel kinases were identified from the public EST databases using a Hidden Markov model, abbreviated HMM (Krogh, A., Brown, M., Mian, I. S., Sjolander, K., and Haussler, D. 1994. Hidden Markov models in computational biology: Applications to protein modeling. J. Mol Biol., 235:1501-1531). The model was built with 70 mammalian and yeast kinase catalytic domain sequences. These sequences were chosen from a comprehensive collection of kinases such that no two sequences had more than 50% sequence identity. ESTs were tnanslated in six open reading frames and were searched against the model. ESTs that had a score of at least 10 against the HMM were then masked for repetitive sequences and vectors and were clustered using MSA. The resulting contigs were searched against known kinases to identify EST clones that encode novel kinases.

Approximately 40% of the ESTs encoding potentially novel kinases did not correspond to the correct EST upon sequence analysis. Most of these discrepancies were resolved by ordering additional clones, however, 14 remained unavailable. These 14 ESTs were amplified from a variety of single-stranded cDNA sources with primers derived from the corresponding EST entry as shown on Table 5. The PCR product was subcloned into a bluescript vector, digested to confirm the presence of a correct size insert and sequenced. Full sequencing of EST and PCR was carried out using a cycle sequencing Big-dye kit with AmpliTaq DNA Polymerase, FS (ABI, Foster City, Calif.). Sequencing reaction products were run on an ABI Prism 377 DNA Sequencer.

TABLE 5

Primers used to clone PCR products corresponding to novel
kinases

| | | | | | |
|---|---|---|---|---|---|
| | ID# | ID# | Parent | 5′ primer | 3′ primer |
| sp | na | aa | Sequence | Sequence* | Sequence* |

| | | | | | |
|---|---|---|---|---|---|
| H | 33 | 153 | 2R22-5-11 | GAGATCGRNTTYAARGA | TGTCACNCCNAGNSWCCAN |
| | | | | RTTYGA | AYRTT |
| M | 81 | 200 | 5R57_10_2_{—} | GCTGCTGGACAGTGACT | GAAAGCAAAGCCTTCACAC |
| | | | m TESK2_m | TGTATTT | CTT |
| H | 67 | 187 | SR69_17_2_h | CTCTCACCTCAGGAACT | GCTTGCGGATCTTCTCA |
| | | | | GG | |
| H | 46 | 166 | SGK309_h | GACATCCTGCCGGCCAA | CGGCCCTGGAGCTGCATCA |
| | | | | CTACG | CTA |
| M | 67 | 228 | 5R72_16_2_h | TGCGCGACACCATTGAC | CTCAGGGCTTACATACAGA |
| | | | | CAG | G |
| H | 45 | 165 | 5R72_8_2_h | AAAGGAGAACTACATTT | CTTCATCATCTCTAATACAT |
| | | | | TGAAAAT | TGGTTGG |
| H | 41 | 161 | Z36720 | CAAATTAAGATCATTGA | GGAAACAAAGTCCTTGGCC |
| | | | | CTTTGGG | TC |
| H | 115 | 234 | AL031652- | GTGGACATCTGGTCCCT | GTAGGTCCTTCACTCTTGG |
| | | | Pak6 | CG | AG |

*degenerate oligonucleatide residue designation:
N = A,C,G ot T
R = A or G
Y = C or T
S = C or G
W = A or T

Full-length Sequence Extension of Protein Kinases Using cDNA and Genomic Databases

Extension of partial cDNA sequences to encompass the full-length open-reading frame was carried out by iterative blastn searching of the cDNA databases listed in Table 6. All blastn searches were conducted using a blosum62 matrix, a penalty for a nucleotide mismatch of −3 and reward for a nucleotide match of 1. The gapped blast algortihm is described in: (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402).

TABLE 6
Databases used for cDNA-based sequence extensions

| Database | Database Date |
|---|---|
| LifeGold templates | February 2000 |
| LifeGold compseqs | February 2000 |
| LifeGold compseqs | March 2000 |
| LifeGold compseqs | April 2000 |
| LifeGold fl | February 2000 |
| LifeGold flft | April 2000 |
| NCBI human Ests | May 2000 |
| NCBI murine Ests | May 2000 |
| NCBI nonredundant | May 2000 |

Extension of partial cDNA sequences to encompass the full-length open-reading frame was also carried out by iterative searches of genomic databases. Three methods were used. The first method made use of the Smith-Waterman algorithm to carry out protein-protein searches of the closest homologue or orthologue to the partial kinase. The target databases consisted of Genescan and open-reading frame (ORF) predictions of all human genomic sequence derived from the human genome project (HGP) as well as from Celera. The complete set of genomic databases searched is shown in Table 7 below. Genomic sequences encoding potential extensions were further assessed by blastp analysis against the NCBI nonredundant to confirm the novelty of the hit. The extending genomic sequences were incorporated into the cDNA sequence after removal of potential introns using the Seqman program from DNAStar. The default parameters used for Smith-Waterman searches were as shown next. Matrix: blosum 62; gap-opening penalty:12; gap extension penalty:2. Genescan predictions were made using the Genescan program as detailed in (Chris Burge and Sam Karlin “Prediction of Complete Gene Structures in Human Genomic DNA”, JMB (1997) 268(1):78-94). ORF predictions from genomic DNA were made using a standard 6-frame translation.

The second method for genomic sequence-based extensions made use of tBlastn searches of the homologue or orthologue to the partial kinase against the cDNA databases listed in Table 7. The recognition of significant hits in these databases made possible to identify bridging partial cDNA clones. The iterative application of the two methods made possible the assemblage of the virtual full-length sequence for a large number of the kinases presented in this application. All tblastn searches were conducted using a blosum62 matrix, a penalty for a nucleotide mismatch of −3 and reward for a nucleotide match of 1.

The last method for defining cDNA extensions from genomic sequence used iterative searches of genomic databases through the Genescan program to predict exon splicing and the Genewise program (http://www.sanger.ac.uk/SoftwarefWise2/) to predict potential ORFs based on homology to the closest orthologue/homologue. Table 7. Databases used for genomic-based sequence extensions

TABLE 7
Databases used for genomic-based sequence extensions

| Database | Number of entries | Database Date |
|---|---|---|
| Celera v. 1-5 | 5,306,158 | Jan 19/00 |
| Celera v. 6-10 | 4,209,980 | Mar 24/00 |
| Celera v. 11-14 | 7,222,425 | Apr 24/00 |
| Celera v. 15 | 243,044 | May 14/00 |
| HGP all Genescan | 25,885 | Apr 04/00 |
| HGP; Phase 0 | 4,944 | May 04/00 |
| HGP; Phase 1 | 28,478 | May 05/00 |
| HGP; Phase 2 | 1,508 | May 04/00 |
| HGP; Phase 3 | 9,971 | May 05/00 |

Virtual Extensions

Human AA826850 (SEQ ID NO: 3, SEQ ID NO:124)

Blastn analysis of the partial AA826850 sequence revealed an extension to encompass the complete ORF in the Incyte EST 238299.1. A frame-shift correction at position 595 of this EST (marked by X in NA sequence) generated an uninterrupted ORF.

Human AA960957 (SEQ ID NO: 4, SEQ ID NO:125)

Since the initial filing of this application, the partial AA960957 sequence appeared in the public database as the full-length gene for a protein kinase encoded by a gene that maps adjacent to the evc (AJ250839) (ellis-van creveld syndrome and weyers acrodental dysostosis) gene from 4p 16.1.

Human 5R79-46-1_h (SEQ ID NO:5, SEQ ID NO:126)

Blastn analysis of the partial 5R79-46-1 sequence revealed an extension to encompass the complete ORF in the Incyte EST 463894.6. Since the initial filing of this application, the full-length virtual 5R79-46-1 appeared in the public database as the full-length gene for the TANK-binding kinase (TBK1) (Pomerantz, J. L. and Baltimore, D. (1999) EMBO J. 18 (23), 6694-6704). TBK1 participates in NF-kB activation through the formation of a signaling complex with TRAF2 and TANK.

Human AA305176 (SEQ ID NO: 6, SEQ ID NO:127)

Blastn analysis of the partial AA305176 sequence revealed an extension to encompass the complete ORF in the Incyte EST 220937.1.

Human AA256100 (SEQ ID NO: 8, SEQ ID NO:129)

Blastn analysis of the partial AA256100 sequence revealed an extension to encompass the complete ORF through the assembly of three partial clones: Incyte EST 480815.6, KIAA0965 (BAA76809) and AA256100.

Human AA210825 (SEQ ID NO: 9, SEQ ID NO: 130)

Blastn analysis of the partial AA210825 sequence revealed an extension to encompass the nearly complete ORF through the assembly of three partial clones: Incyte EST 014721.7, and the NCBI EST's AW01158 and AA210825. An insertion of two “N's” at positions 1915 and 1916 generated an uninterrupted ORF. Blastx analysis indicated the possibility of a start Met in the range of 400-450 nucleotides (i.e. compared to the closest homolog, human PKCmu (CAA53384.1). However, no Met was found in this region; rather ORF ends in an in-frame stop preceeded by the sequence “RGLLAPGDPPCPPPNPAPATPPSSRLPTELFSNFCDS”. It is possible that part of the sequence covered by nucleotide positions 1-400 derived from AW01158 comes from an intron, explaining the absence of a start Met.

Human AA127299 (SEQ ID NO:10, SEQ ID NO:131)

No entries in the database extended this sequence. The 1684 bp insert of this EST contains a 1369 bp intron at the 3′ end. Blastx and SW analysis of the 315 bp coding region revealed homology to the extracatalytic C2 domain of PKC. This EST, may or may not encode a kinase.

Human AA316804 (SEQ ID NO:11, SEQ ID NO:132)

Since the initial filing of this application, the partial AA316804 sequence appeared in the public database as the full-length gene for the PKC family protein kinase EPK2 or PKCnu (AB015982).

Human H19102 (SEQ ID NO:14, SEQ ID NO:135)

Genewise and Genescan analyses of the partial H19102 sequence revealed an extension from the HGP phase 3 contig 3810672 to encompass the complete catalytic domain of this EST. Blastn analysis against the non-redundant database revealed that this gene is found in the cosmid AC005726 from chromosome 17.H19102 may encode a dual catalytic kinase given the homology to S6 kinase. Analysis of genomic sequence upstream of the 5′ end of H19102 revealed a non-kinase gene oriented in the same polarity as H19102 suggestive of the start Met for H19102 being close to the 5′ end of the H19102 sequence. From this analysis it is deduced that the second catalytic domain of H19102, if present, is most likely located within the 47334-185,215 bp region of the genomic sequence of AC005726.

Human AA476563 (SEQ ID NO:15, SEQ ID NO:136) Since the initial filing of this application, the partial AA476563 sequence appeared in the public database as the full-length gene for the protein kinase RPS6KC1 (NM_{—}012424)(Zhang, H. et al Genomics (1999) 61, 314-318), which is an S6 kinase mapping to 12q12-q13.1.

Human AA626690 (SEQ ID NO:16, SEQ ID NO:137)

Since the initial filing of this application, the partial AA626690 sequence appeared in the public database as the full-length gene for the protein kinase RPS6KA6 (AF184965) (Yntema H. G et al (1999) Genomics 62, 332-343), an S6 kinase commonly deleted in patients with complex X-linked (Xq21.1) mental retardation.

Human AI215680 (SEQ ID NO: 17, SEQ ID NO:138)

Since the initial filing of this application, the partial AI215680 sequence appeared in the public database as the full-length gene encoding a hypothetical protein (AAD30182) from the locus AC006530.4 from chromosome 14.

Human AA887783 (SEQ ID NO:21, SEQ ID NO:142)

Blastn analysis of the partial AA887783 sequence revealed an extension to encompass the nearly complete ORF through the assembly of three partial clones: Incyte 415390R6 and the NCBI EST's AA887783 and N94726. Since the initial filing of this application, the nearly full-length virtual AA887783 sequence appeared in the public database as the fill-length gene encoding SGK3 (AF169035), a serum- and glucocorticoid-induced protein kinase (Kobayashi, T. et al (1999) Biochemical J. 344, 189-197.

Human R47805 (SEQ ID NO:22, SEQ ID NO:143)

A cDNA clone encoding the full-length ORF of R47805 was isolated using R47805 as a screening probe. A full-length form for R47805 has also appeared in the public database as

PTK9L (NM_{—}007284), an A6-related protein kinase.

Human H60215 (SEQ ID NO:23, SEQ ID NO:144)

Blastn analysis of the partial H60215 sequence revealed an extension to encompass the complete ORF in the public EST AI275726. This was confirmed through the full insert sequencing of this EST (2,310 bp) which corresponds to the sequence under SEQ ID NO:144.

A different stop codon was predicted for AI275726 compared to H60215 due to a single nucleotide insertion at position 1586 in AI1275726. Evidence for the extra nucleotide comes from EST AI191922.

SGK324_{n—}h orthologue of W30246_m (SEQ ID NO:24, SEQ ID NO:145)

Blastn, blastx and Smith-Waterman analyses of genomic databases revealed an extension to encompass the complete ORF corresponding to the human orthologue of murine W30246. Exons predicted from the following sequences were used for contig construction: Celera 17000189645083, 17000057549105 and 11000501939981; Incytel42404.1, HGP_{—}7249119, Incyte 7196489H1, Celera 11000501939981, 17000028165594; Incyte 7249119_{—}3, Celera 17000035772368, 11000502081575 and 17000140274329. The latter Celera sequence provides the N-terminus.

Human AA383293 (SEQ ID NO:26, SEQ ID NO:147)

Blastn, blastx and Smith-Waterman analyses of genomic databases revealed an extension to encompass the complete ORF corresponding for AA383293. Exons predicted from the following sequences were used for contig construction: (numbers in parenthesis refer to the aa sequence of the closest homolog (RU2S, NP_{—}057440) used for the Smith-Waterman query): N-term from Incyte 6010175_{—}2 (14-97), Incyte 6981981 (134-184) 7596749 (186-232) Celera 17000020789545 (243-301) CAB75619.1 (310-341)-(56-145 DCX homology) 6010175_{—}2, Celera 17000030058129 (241-262 DCX homology).

Human AA021445 (SEQ ID NO:32, SEQ ID NO:152)

Blastn analysis revealed an extension to encompass the nearly complete ORF corresponding for AA021445. Contig reconstruction was as follows: nucleotides 1-802 from KIAA0999 (AB023216); nucleotides 803-4321 from full-insert sequence of AA021445. A pairwise alignment between the AA021445 and KIAA0999 revealed three inserts in the extracatalytic C-terminus of 48, 48 and 161 aminoacids. In addition, both AA021445 and KIAA0999 have 15 copies of a CAG repeat. Trinucleotide repeats are often found in genes that linked to neurodegenerative diseases.

Human 2R22-55-1 (SEQ ID NO:33, SEQ ID NO:153)

Blastn analysis revealed an extension in the Incyte EST clone 321074.1 to encompass the complete ORF corresponding to 2R22-55-1.

Human orthologue of AA544838_m (SEQ ID NO:36, SEQ ID NO:156)

tBlastn analysis identified the partial human KIAA0135 (U79240) clone as the human orthologue of murine AA544838. Blastn revealed an extension KIAA0135_h (U79240) to encompass the complete ORF. The full ORF was reconstructed from Incyte406786.5, KFZp430051 and KIAA0135 (U79240).

Human orthologue of AI785735_m (SEQ ID NO:38, SEQ ID NO:158)

tBlastn analysis identified the partial human KIAA0781 (AB018324) clone as the human orthologue of murine AI785735. Blastn revealed an extension KIAA0135_h (U79240) to encompass the complete ORF. The full ORF was reconstructed from Incyte 986123.37 KIAA0781 (AB018324).

Human AA207220 (SEQ ID NO:39, SEQ ID NO:159)

Blastn analysis revealed an extension to encompass the nearly complete ORF corresponding for AA021445. The full ORF was reconstructed from Incyte 402740.1 and AA207220. Frame corrections: deletion of 441 and 595 over Inc402740.1 seq based on blastx to keep frame open; two n insertions 940, 941 over AA207220 to keep frame open.

Human AA426580 (SEQ ID NO:40, SEQ ID NO:160)

Since the initial filing of this application, the partial AA426580 sequence appeared in the public database as the full-length gene encoding MAK-V (AJ271722) from chromosome 21q22.1.

Human 5R79-54-1 (SEQ ID NO: 41, SEQ ID NO:161)

Genewise and Genescan analyses of the partial 5R79-54-1 sequence revealed an extension from genomic sequence to encode the full ORF for SR79-54-1.

Human orthologue of AA542015_m (SEQ ID NO: 42, SEQ ID NO:162)

tblastn analysis identified KIAA1297 (AB037718). Blastn extended the KIAA1297 sequence to provide the C-terminus through the Incyte 224074.1 EST. The partial ORF consists of a dual catalytic domain flanked by 6 Ig domains and 2 fibronectin repeats. Based on homology-to the bt drosophila protein (AAF59316.1), the human form of AA542015 is expected to be missing 16 Ig domains.

Human R19772 (SEQ ID NO:44, SEQ ID NO:164)

The full-length ORF for R19772 was isolated by screening a cDNA library using a probe derived from RI 9772. Since the initial filing of this application, the R19772 sequence appeared in the public database as the full-length gene encoding Trio (Duet) (AB011422). CDNA library screening revealed multiple isoforms for this gene which are summarized in the Table below.

TABLE 8
Isoforms for R19772

| | Kestrl | | | |
|---|---|---|---|---|
| Kestrl | AA | Isoform | | |
| Name | Acc # | type | Source | Description* |
| Trad | R19772 | B | Skeletal | Deletion of K at 124 |
| (Duet) | | | muscle | |
| | | | | Deletion of Q at 616 |
| | | | | Substitution of E for G at |
| | | | | 762 |
| | | C | Skeletal | Deletion of K at 124 |
| | | | muscle | |
| | | | | Deletion of Q at 616 |
| | | | | Substitution of E for G at |
| | | | | 762 |
| | | | | Deletion of 32 aa (160-191) |
| | | D | Lung tumor | Deletion of Q at 616 |
| | | | | Deletion of 32 aa (160-191) |
| | | E | Lung tumor | Deletion of Q at 616 |
| | | | | Deletion of 32 aa (160-191) |

*reference amino acid position are with respect to sequence of Trad (AB011422)

Human AA435956 (SEQ ID NO:48, SEQ ID NO:168)

Blastn analysis revealed an extension to encompass the nearly complete catalytic region of AA435956. 5′ end sequence extension was provided by genomic locus AC007242.3_h (range 44880-43801). Based on blastx analysis, the extended sequence encodes is full-length at the C-terminus.

Human AA397553 (SEQ ID NO: 51, SEQ ID NO:171)

Since the initial filing of this application, the partial AA397553 sequence appeared in the public database as the full-length gene encoding CRK7 (AF227198), a novel CDC2-related protein kinase that colocalizes with interchromatin granule clusters.

Human AA789239 (SEQ ID NO: 52, SEQ ID NO:172)

Since the initial filing of this application, the partial AA789239 sequence appeared in the public database as the full-length gene encoding NKIAMRE (AF130372), a novel kinase deleted in human leukemia

Human AA631990 (SEQ ID NO:55, SEQ ID NO:175)

Blastn analysis revealed an extension to encompass the full-length ORF for AA631990. The full ORF was reconstructed from 253847.5 and AA631990 and AA207220. Frame corrections: delete 1 C at 1380, delete 2N's at 2033/2034.

Human AA557536 (SEQ ID NO:56, SEQ HD NO:176)

Blastn analysis revealed an extension to encompass full-length ORF for AA557536. The full ORF was reconstructed from AA557536, celera 11000504061899 and the Incyte 097089.1 EST. An 85 bp intron was removed from AA557536.

Human N34132 (SEQ D) NO: 63, SEQ ID NO:183)

Full sequencing of EST N34132 (1.3 kb) confirmed that this cDNA encodes a novel NEK-subfamily kinase. Blast analysis against the EST database showed that four EST sequences (AA283140, AA283140, AA282911 and N53011) extended the sequence of N34132 at the 3′ end to form a 2.31 kb contig. Blast analysis of the new contig against the nonredunat public database showed that the N34132 extended contig overlapped (100% identity) over 228 bp at its 3′ end with human KIAA0344 (AB002342), a 5,787 bp cDNA encoding a 1246 aa polypeptide. The 5′790 bp of the KIAA0344 cDNA (encoding the 58 N-terminal protein sequence) were found to be divergent with respect to the extended 2.32 kb N34132 contig. Evidence that the extended N34132 contig (2.31 kb) and KIAA0344 (AB002342) belong to the same gene is the following. First, blast analysis of the nucleotide sequences for N34132 and KIAA0344 against the NRN database confirmed that these cDNA's are transcribed from the same genomic locus defined by two overlapping BACs (AC004765 and AC004803) from chromosome 12p 13.3. Second, full sequence determination of a PCR fragment amplified from single-stranded cDNA confirmed the junction between the extended N34132 contig and KIAA0344_h (AB002342). The 462 PCR product was amplified with primers CTCCTCAACAGACAGTGCAG (5′ primer) and GACATTCTACTACTCGGTCTC (3′ primer) designed from the N34132 extended contig and KIAA0344 sequences, respectively. The region of N34132 containing the start Met was isolated by PCR from a testis cDNA library (Clontech).

Human 5R69-17-2 (SEQ ID NO:67, SEQ ID NO:187)

The full-length ORF for 5R69-17-2 was isolated by screening a cDNA library using a probe derived from 5R69-17-2.

Human H85811 (SEQ ID NO:68, SEQ ID NO:188)

Tblastn, Smith-Waterman and blastn analyses using cDNA databases revealed an extension to encompass full-length ORF for H85811. The full ORF was reconstructed from Incyte ESTs 202971.8, 034583.3 and 034583.1 and public ESTs H85811 and AI570599.

Human R43524 (SEQ ID NO:73, SEQ ID NO:192)

Blastn analysis revealed an extension to encompass the complete catalytic region and the C-terminus of R43524. Since the initial Sling of this application, the partial R43524 sequence appeared in the public database as the full-length gene encoding the heme-regulated initiation factor 2-alpha kinase (HRI) (AF1 81071).

Human AA088547 (SEQ ID NO:78, SEQ ID NO:197)

Genewise and Genescan analyses of genornic databases revealed an extension to encompass the complete ORF for AA088547.

Human orthologue of AA139478_m (SEQ ID NO:80, SEQ ID NO:199) Tblastn identified the Incyte 211475.1 as the potential full-length human orthologue of murine AA139478

Human AA232253 (SEQ ID NO:82, SEQ ID NO:201)

The full-length ORF for AA232253 was isolated by screening a cDNA library using a probe derived from AA232253. Since the initial filing of this application, the AA232253 sequence appeared in the public database as the full-length gene encoding SLK (ABO11422). SLK is a stress-regulated mixed lineage kinase-like protein that activation of Rac and induction of apoptosis. cDNA library screening revealed multiple isoforms for this gene which are summarized in the Table below.

TABLE 9
Isoforms for AA232253

| Kestrl | Kestrl AA | Isoform | |
|---|---|---|---|
| Name | Acc # | type | Description* |
| MLK4 | AA232253 | MLK4 | Substitution of C for W at 346 |
| | | MLK4B | Different Cterm (332-800); seq in |
| | | | MLK4B is as shown in* |

*C-terminus specific to MLK4B

LPLAARMSEESYFESKTEESNSAEMSCQITATSNGEGHGMNPSLQAMMLMGFGDI
FSMNKAGAVMHSGMQINMQAKQNSS
KTTSKRRGKKVNMALGFSDFDLSEGDDDDDDDGEEEDNDMDNSE

Human H97685 (SEQ ID NO:84, SEQ ID NO:203)

Blastn analysis revealed an extension to encompass the full-length ORF for H97685. The full ORF was reconstructed from Incyte 474824.1 and the public ESTs H97685 and M62021.

Human AI052250 (SEQ ID NO:87, SEQ ID NO:206)

Blastn analysis revealed an extension to encompass the full-length ORF for AI052250. The fall ORF was reconstructed from Incyte 396868.1, the public partial cDNA FLJ10074 (minus intron) and the public ESTs and the public ESTs AI052250 and H97685, AI499220 and M62021.

Human AA278842 (SEQ ID NO:88, SEQ ID NO:206)

A nearly full-length cDNA (FL4F12) for AA278842 was isolated by screening a cDNA library using a probe derived from AA278842. A full-length virtual ORF was generated using FL4AF12 and AA278842.

Human AA599286 (SEQ ID NO:89, SEQ ID NO:208)

Since the initial filing of this application, the partial AA599286 sequence appeared in the public database as a full-length ORF (AK000342).

Human AA425725 (SEQ ID NO:90, SEQ ID NO:209)

Since the initial filing of this application, the partial AA425725 sequence appeared in the public database as MSSK1, a serine kinase gene located from human chromosome Xq28.

Human SGK022 orthologue of AA060026_m (SEQ ID NO:91, SEQ ID NO:210)

Tblastn, Smith-Waterman and blastn analyses of cDNA and genomic databases databases revealed a potential human orthologue for murine AA060026. The full-length ORF for SGK022 was reconstructed from genomic locus AC022307.

Human AA399669 (SEQ ID NO:93, SEQ ID NO:212)

Blastn analysis revealed an extension to encompass the full-length ORF for AA399669. The full ORF was reconstructed as follows: sequence 1-1007 from AL136295.2; sequence1008-2319 from AA399669 and Incyte 428177.1.

Human AA883975 (SEQ ID NO:95, SEQ ID NO:214)

Genescan and Genewise analyses of the genomic databases revealed an extension for AA883975 to encompass the full-length ORF

Human AA905446 (SEQ ID NO:96, SEQ ID NO:215)

Tblastn, Smith-Waterman and blastn analyses of cDNA and genomic databases databases revealed an extension for AA905446 to encompass the full-length ORF. For the Smith-Waterman analysis murine STK22 ( NP_{—}033462) was used as the closest orthologue. Contig formation: range 162133-163687 from HGP_h 6921333_{—}9; removed intron (146-893) predicted from blastx analysis.

Human H29974 (SEQ ID NO: 97 SEQ ID NO:216)

Blastn analysis revealed an extension to encompass a complete catalytic ORF for AA399669. The nearly full-length ORF was reconstructed using Incyte 213829.1 and H29974.

Human AA215311 (SEQ ID NO:99, SEQ ID NO:218)

Blastn analysis revealed an extension to encompass the full-length ORF for AA21531. The full ORF was reconstructed from Incyte 067584.1, 022456.1, AA215311 and the reverse complement of CPG_{—}043208.

Human AA018361 (SEQ ID NO:100, SEQ ID NO:219)

The full-length ORF for AA018361 was isolated by screening a cDNA library using a probe derived from AA018361. This yielded clone Sug4-30. Clone Sug4-30, like multiple, independent cDNA clones contained a 181 bp intron. The existence of intron-less RNA's was confirmed by a PCR reaction that generated a product that upon sequence analysis skipped the intron region. The full-length virtual ORF for AA018361 was generated through a contig between AL117482 (seq 1-367) and the sequence for clone Sug4-30.

Human orthologue of AA396601_m (SEQ ID NO:106, SEQ ID NO:225)

tBlastn and Smith-Waterman analyses of genomic sequence revealed an extension to encompass the full catalytic region for the human orthologue of AA396601. The ORF was reconstructed from Incyte 018653.9 (7261449H1, 6891740J1) and genomic sequence CPG_{—}040010.

Human orthologue of AA671275_m (SEQ ID NO:108, SEQ ID NO:227)

Since the initial filing of this application, a potential human orthologue for murine AA671275 appeared in the public database as the full-length ORF for vaccinia related kinase 3 (BAA90769).

Human H05721 (SEQ ID NO:111, SEQ ID NO:230)

Genescan and Genewise analyses of genomic sequence revealed an extension to encompass the full-length ORF for H05721.

Human AI086865 (SEQ ID NO:112, SEQ ID NO:231)

Genescan and Genewise analyses of genomic sequence revealed an extension to encompass the full-length ORF for AI086865. The full-length ORF was reconstructed from Celera 17000102901516, Incyte 243269.1 and public AL1377531.

Human AA836348 (SEQ ID NO:113, SEQ ID NO:232)

Genescan and Genewise analyses of genomic sequence revealed an extension to encompass the full-length ORF for AA836348.

Human R86668 (SEQ ID NO:14, SEQ ID NO:233)

The full-length ORF for R86668 was isolated by screening a cDNA library using a probe derived from R86668. Since the initial filing of this application, the R8668 sequence appeared in the public database as the full-length gene mitogen-activated protein kinase kinase kinase 6 (MAP3K6)(NM_{—}00467).

Human 2R41-9-4 (SEQ ID NO:16, SEQ ID NO:235)

The full-length virtual ORF for 2R41-9-4 was generated using genomic sequence to provide the Nterminus for the partial ORF predicted from clone 2R41-9-4

TABLE 10
Sequences deleted from the provisional patent due
to duplication with other genes in the patent

| Prov. SEQ ID NO: (na) | Prov. SEQ ID NO: (aa) |
|---|---|
| 160 | 196 |
| 213 | 214 |
| 215 | 216 |
| 122 | 126 |
| 119 | 123 |
| 148 | 184 |
| 4 | 20 |
| 7 | 23 |
| 205 | 206 |
| 14 | 30 |
| 15 | 31 |
| 35 | 56 |
| 42 | 63 |
| 51 | 72 |
| 44 | 65 |
| 77 | 91 |
| 78 | 92 |
| 79 | 93 |
| 80 | 94 |
| 157 | 193 |

Results

Table 1 documents the results from the analysis of the nucleic acid sequence data. From left to right the data presented is as follows. “Gene name” refers to the EST or PCR fragment that defined the novel kinase. “Species” refers to the organism the sequence was derived from. “ID#” refers to the nucleic acid and amino acid sequence ID number designation from this patent. “Kinase family” and “Kinase group” refers to the protein kinase classification defined by sequence homology and based on previously established phylogenetic analysis [Hardie, G. and Hanks S. The Protein Kinase Book, Academic Press (1995) and Hunter T. and Plowman, G. Trends in Biochemical Sciences (1977) 22:18-22 and Plowman G. D. et al. (1999) Proc. Natl. Acad. Sci. 96:13603-13610)]. “ORF Start”, “ORF End”, “ORF Length” refer to the open reading frame range and length as calculated by standard nucleic acid translation programs such as MapDraw (DNAStar). “DNA Repeats” refers to regions of low complexity sequence or repetitive elements such as Alu, LINE, SINE, and LTR sequences. The chromosomal location (CHR localization) for 37 of the 110 novel protein kinases is shown on Table 1 (NA, not available). The methods for determining chromosomal position are outlined below, in Example 2.

Table 2 documents the results from the analysis of the amino acid sequence data. From left to right the data presented is as follows. “Gene name” refers to the EST or PCR fragment that defined the novel kinase. “Species” refers to the organism the sequence was derived from. “ID#” refers to the nucleic acid and amino acid sequence ID number designation from this patent. “Kinase family” and “Kinase group” refers to the protein kinase classification defined by sequence homology and based on previously established phylogenetic analysis [Hardie, G. and Hanks S. The Protein Kinase Book, Academic Press (1995) and Hunter T. and Plowman, G. Trends in Biochemical Sciences (1977) 22:18-22 and Plowman G. D. et al. (1999) Proc. Natl. Acad. Sci. 96:13603-13610)]. “nraa Score”, “ID match aa”, “Identity”, “Similar”, “nraa Match Acc#”, Description” refer to the data obtained using a Smith-Waterman search of the amino acid sequence against the non-redundant protein database (Matrix: Pam100; gap open/extension penalties 14/1). “Kinase Domain Start”, “Kinase Domain End”, “Profile Start” and “Profile End” refer to data obtained using a Hidden-Markov Model to define catalytic range boundaries. The profile has a length of 261 amino acids, corresponding to the complete protein kinase catalytic domain Proteins in which the profile recognizes a full length catalytic domain have a “Profile Start” of 1 and a “Profile End” of 261. The boundaries of the catalytic domain within the overall protein are noted in the “Kinase Domain Start” and “Kinase Domain End” columns.

The following abbreviations were used for kinases:
ASK Apoptosis signal-regulating kinase
[symbol] K Ca2+/calmodulin-dependent protein kinase
[symbol] RK Cell cycle-related kinase
CDK Cyclin-dependent kinase
CK Casein kinase
DAPK Death-associated protein kinase
DM myotonic dystrophy kinase
Dyrk dual-specificity-tyrosine phosphorylating-regulated kinase
GAK Cyclin G-associated kinase
GRK G-protein coupled receptor
GuC Guanylate cyclase
HEPK Homeodomain-interacting protein
IRAK Interleukin-1 receptor-associated kin
MAPK Mitogen activated protein kinase
MAST Micotubule-associated STK
MLCK Myosin-light chain kinase
MLK Mixed lineage kinase
NIMA NimA-related protein kinase
PKA cAMP-dependent protein kinase
RSK Ribosomal protein S6 kinase
RTK Receptor tyrosine kinase
SGK Serum and glucocorticoid-regulated kinase
STK serine threonine kinase
ULK UNC-51-like kinase

The following abbreviations were used for species
H Human
M Murine
R Rat
FV Fowlpox virus
MT M. thermoautotrophicum
CE Caenorhabditis elegans
DM Drosophila melanogaster
OS Oryza sativa
SP Schizosaccharomyces pombe
TP Tetrahymena pyriformis
PI Petunia inflata
NC Neurospora crassa
MSV Medicago sativa
MSV Moloney murine sarcoma virus
SA Squalus acanthias
CS Cucumis sativus
GM Glycine max
LL Lilium longiflorum
TV Trichomonas vaginalis
MP Mycoplasma pneumoniae
DD Dictyostelium discoideum
SC Saccharomyces cerevisiae
MT Methanobacterium thermoautotrophicum
   Domain and Motif Identification

A Hidden Markov model (HMM) (Krogh, A., Brown, M., Mian, I. S., Sjolander, K, and Haussler, D. (1994). Hidden Markov models in computational biology: Applications to protein modeling. J. Mol. Biol., 235:1501-1531) was used to identify, both catalytic and extracatalytic domains. Table 4 shows extra-catalytic domains that were identified using the HMM program. Other domains such as coiled-coil and pest motifs were identified as described next.

Potential coiled-coil domains were identified using the COILS program (www.ch.embnet.org/software/COILS_form.html). The matrix used was MTIDK with windows of 14, 21, 28 amino acids. Only regions scoring 0.5 or higher were considered to have potential coiled-coil domain region.

Protein sequences containing potential pest motifs were identified using the program PESTfind (www.at.embnet.org/embnet/tools/bio/PESTfind/). PEST regions in proteins are by definition sequences that tend to be rich in proline, glutamic or aspartic acid, argininine and histidine; they have been associated with increased protein turnover rates (Rogers S. et al. (1986) Science 234, 364-368. The algorithm defines PEST sequences as hydrophilic stretches of amino acids greater than or equal to 12 residues in length. Such regions contain at least one P, one E or D and one S or T. They are flanked by lysine (K), arginine (R) or histidine (H) residues, but positively charged residues are disallowed within the PEST sequence. PESTfind produces a score ranging form about −50 to +50. By definition, a score above zero denotes a possible PEST region; a value greater than +5 defines a high probability that there is a PEST domain.

Identification of Potential Coiled-Coil Domains and PEST Domains in N34132

Potential coiled-coil domains were identified in N34132 (SEQ ID NO:183) using the COILS program. Only regions scoring 0.5 or higher were considered to have potential coiled-coil domain region. The amino acid positions within N34231 scoring for potential coil-coil regions are shown below.

TABLE 11
coiled-coil domains predicted for N34132

| Coiled-coil Region | Amino acid range | Length (aa) |
|---|---|---|
| 1 | 124-147 | 24 |
| 2 | 437-451 | 15 |
| 3 | 495-526 | 32 |
| 4 | 1,723-1,749 | 27 |

Potential PEST domains were identified in N34132 using PESTfind, a value greater than +5 defines a high probability that there is a PEST domain. The amino acid positions within N34132 scoring for potential PEST regions are shown below.

TABLE 12
Potential Pest domains identified in N34132

| PEST Region | Score | Amino acid range | Amino Acid Length |
|---|---|---|---|
| 1 | +4.91 | 54-95 | 42 |
| 2 | +11.4 | 537-570 | 34 |
| 3 | +31.08 | 1293-1304 | 12 |
| 4 | +10.15 | 1543-1565 | 23 |
| 5 | +6.17 | 1698-1732 | 35 |

### Example 2

### Chromosomal Localization of Novel Mammalian Protein Kinases Materials and Methods

Several sources were used to find information about the chromosomal localization of each of the genes described in this patent. First, the accession number for the nucleic acid sequence was used to query the Unigene database. The site containing the Unigene search engine is: http://www.ncbi.nlm.nih.gov/UniGene/Hs.Home.html. Information on map position within the Unigene database is imported from several sources, including the Online Mendelian Inheritance in Man (OMIM, http://www.ncbi.nlm.nih.gov/Omnim/searchomim.html), The Genome Database (http://gdb.infobiogen.fr/gdb/simpleSearch.html), and the Whitehead Institute human physical map (http://carbon.wi.mit.edu:8000/cgi-bin/contig/sts_info?database=release). For example, searching Unigene with W56561, an EST for a MAK-like kinase, the following information is retrieved: Chr.14, D14S65-qTEL. The location of this gene on an “ideogram” of the cytogenetic map of chromosome 14 is also provided, showing that W56561 maps to the bottom of chromosome 14, between 14q31 and 14qTel. If Unigene has not mapped the EST, then the nucleic acid for the gene of interest is used as a query against databases, such as dbsts and htgs (described at http://www.ncbi.nhn.nih.gov/BLAST/blast_databases.html) containing sequences that have been mapped already. The nucleic acid sequence is searched using BLAST-2 at NCBI (http://www.ncbi.nlm.nih.gov/cgi-bin/BLAST/nph-newblast) and is used to query either dbsts or htgs. In addition to the Whitehead and GDB sites mentioned above, Stanford University maintains a useful site for chromosomal mapping from STS data (http://www-shgc.stanford.edu/RH/rhserverformnew.html). Matches in htgs are often resolved immediately because the genomic region hit is annotated in the htgs entry. If an [symbol] match is found (defined roughly as 99% identity over a region of about 100 [symbol] pairs or longer, excluding any repetitive sequence), then the mapped position of the entry in the database is assigned to the original kinase query. Once a cytogenetic region has been identified by one of these approaches, disease association is established by searching OMIM (see above for URL) with the cytogenetic location. OMIM maintains a searchable catalog of cytogenetic map locations organized by disease. A thorough search of available literature for the cytogenetic region is alo made using Medline (http://www.ncbi.nlm.nih.gov/PubMed/medline.html). References for association of the mapped sites with chromosomal abnormalities found in human cancer can be found in: Knuutila, et al., Am J Pathol, 1998, 152:1107-1123.

Results

The chromosomal location for 37 of the 110 novel protein kinases is shown on Table 1. Three of the novel protein kinases were mapped to regions associated with cancer amplicons, as shown on this table. The regions were also cross-checked with the Mendelian Inheritance in Man database, which tracks genetic information for many human diseases, including cancer. References for association of the mapped sites with chromosomal abnormalities found in human cancer can be found in: Knuutila, et al., Am J Pathol, 1998, 152:1107-1123. Association of these mapped regions with other diseases is documented in the Online Mendelian Inheritance in Man (OMIM) (http://www.ncbi.nlm.nih.gov/htbin-post/Omim).

### Example 3

### Generation of Specific Immunoreagents

Materials and Methods

Peptide sequences to extra-catalytic regions of novel kinases are chosen which are not homologous to other known kinases based on a Smith Waterman homology search against the non-redundant protein database and predicted to be antigenic based on the DNAStar Protean program. These peptides are conjugated to KLH using Glutaraldehyde.

Rabbits are immunized with the KLH-peptide conjugates by four injections three weeks apart. The rabbits are bled ten and fourteen days following the third injection and bled out ten days after the fourth. The serum is checked against the peptide by ELISA.

TABLE 13

Peptides to be used as immununogens for raising
antibodies

| Clone | SEQ ID | | Amino |
|---|---|---|---|
| Name | NO (aa) | Peptide Sequence | Location |
| AA8256850 | 124 | KSRDNSRDSSQSEND | 339-353 |
| | | TEKLKRSQDLPREPLP | 372-386 |
| | | RGWRPYDIHS | 223-232 |
| 5R79-46-1 | 126 | FEGPRRNKEVMYK | 224-236 |
| | | KDDYNETVHKKTE | 451-463 |
| | | GTHPKDRNVEKLQ | 541-553 |
| | | EVSKYQEYTNELQET | 643-657 |
| AA256100 | 129 | IDDTSNFDDFPESDI | 405-419 |
| | | TEPDYKSKDWVFL | 427-439 |
| | | EEKKLRRSQHARKET | 61-75 |
| AA210825 | 130 | SNKDTLRKRHYWRLD | 507-521 |
| | | RHTTRKSSTTLRE | 488-500 |
| | | FQNNTTNRYYKEIPL | 528-542 |
| | | GKHRKTGRDVAVK | 668-680 |
| | | FPTKQESQLRNE | 687-698 |
| AA316804 | 132 | ESHVHQEPSKRIPS | 239-252 |
| | | HTKRKSSTMVKEGW | 409-422 |
| | | PSDLDVERDEEAVK | 375-388 |
| | | SPGQGKDHKDLSTSI | 543-557 |
| R47805 | 143 | EPVGRWDQDYDRAVL | 44-58 |
| | | KPKGPGGKRGHKRLI | 325-339 |
| | | PTDVAQLPSRVPRDA | 219-233 |
| AA234451 | 167 | DPFDWEKTGNDGSLT | 293-307 |
| | | HPRPQEKDVWEE | 374-385 |
| | | RENTDEVFPDEQLSD | 340-354 |
| | | RSEITQPDRDIPLVR | 427-441 |
| AA460132 | 180 | LKSYSTSSKKARPVL | 222-236 |
| | | KKLDEVRLRGRKRSM | 237-251 |
| | | ETEKTAQGLSNLAKT | 131-145 |
| N34132 | 183 | SGRRRRPTKSKGSKS | 1848-1862 |
| | | PGTAPSKPPLTKAPV | 1474-1488 |
| | | VDSDTQPKAPGIDD | 1365-1378 |
| | | AHSLDKTSHSSTTGL | 1253-1267 |
| 5R69-17-2 | 187 | GTTREKTDRVKST | 178-190 |
| | | HSEAPELHGKIRSSN | 138-152 |
| | | DETVTPPQFSIV | 87-98 |
| | | QYDVKSEIYS | 204-213 |
| AA278842 | 206 | TVDPEKSVRDQAFKA | 515-529 |
| | | DSSTADRWDDEDWGS | 637-651 |
| | | SVSEDPTQLEEVEKD | 539-553 |
| AA836348 | 232 | NAPTKRPRSSTVTEA | 323-337 |
| | | LDSEEDYYTPQKVDV | 514-528 |
| | | GDKASYRQPKHVEKL | 409-423 |

### Example 4

### Expression analysis of Novel Mammalian Protein Kinases

Gene Expression Analysis

Tissue Arrays

“cDNA libraries” derived from a variety of sources were immobilized onto nylon membranes and probed with 32P-labeled cDNA fragments derived from the gene(s) of interest.

Total RNA or mRNA was used as template in a reverse transcription reaction to generate single-stranded cDNAs (ss cDNA) that were tagged with specific sequences at each end. An oligo dT primer containing a specific sequence (CDS: AAGCAGTGGTAACAACGCAGAGTACT30VN (V=A,G,C N=A,G,C,T)) anneals at the polyA track at the 3′ end of the mRNA and the reverse transcriptase (MMLV RnaseH-) transcribes the antisense strand until it reaches the end of the RNA strand when it adds additional C residues. If a primer (SMII: AAGCAGTGGTAACAACGCAGAGTACGCGGG or ML2G: AAGTGGCAACAGAGATAACGCGTACGCGGG) ending with 3 Gs is added, it anneals to the added Cs and the MMLV recognizes the rest of the primer sequence as template and continues transcription. As a result, the synthesized cDNAs contain specific sequence tags at both the 5′ and the 3′ end. When the 5′ and the 3′ ends are tagged with the same sequence (CDS and SMII) it is referred to as “symmetric.” When the 5′ end is tagged with a different sequence than the 3′ end (CDS and ML2G) is referred to as “asymmetric” A double-stranded “cDNA library” is then generated by PCR amplification using the 3′PCR and ML2 primers (3′ PCR: AAGCAGTGGTAACAACGCAGAGT and ML2:AAGTGGCAACAGAGATAACGCGT) that anneal to the added sequence tags.

The amplified “cDNA libraries” were manually arrayed onto nylon membranes with a 384 pin replicator. The DNA was denatured by alkali treatment, neutralized and cross-inked by WV light. The arrays were pre-hybridized with Express Hyb (Clontech) and hybridized with 32P labeled probes generated by random hexamer priming of cDNA fragments corresponding to the genes of interest. After washing, the blots were exposed to phosphorimaging cassettes and the intensity of the signal was quantified. The amount of the DNA on the arrays was also quantified by treating non-denatured or denatured arrays with Syber Green I or Syber Green II respectively (1:100,000 in 50 mM Tris, pH8.0) for 2 minutes. After washing with 50 mM Tris, pH8.0, the fluorescent emission was detected with a phosphorimager (Molecular Dynamics) and quantified. The amount of the arrayed DNA was used to normalize the hybridization signal and the corrected values are tabulated in Table 3.

Results

The results of the microarray expression analysis of the protein kinases presented in this application is shown in Table 3. Data presentation from left to right is as follows: “Tissue”: tissue type of the cDNA; “Tumor sym”, indicates that the tissue is derived from a tumor, “sym” refers to the fact that the 5′ and 3′ primers used to make the sample are the same; “Normal Sym”, indicates normal tissue was used to make the sample, with symmetric primers as described above; “Tumor lo”, indicates that primary tumor tissue was used to make the cDNA; “Tumor cells”, indicates that these cDNA samples were made from cultured tumor cells; “Normal”, indicates that these samples are derived from normal tissue or cell lines; “Endos”, indicates that these samples are derived from endothelium-related tissue sources; “p53” refers to the status, mutant or wild-type, of the p53 gene in the source samples. Normalized expression values are presented for each gene referred to by its SEQ ID# on the subsequent columns. Genes represented in expression Table 3 are: SEQ ID NO:3 (AA826850), SEQ ID NO:5 (TBK1), SEQ ID NO:6 (AA305176), SEQ ID NO:8 (AA256100), SEQ ID NO:9 (CAB43292), SEQ ID NO:11 (EPK2), SEQ ID NO:12 (PKNbeta), SEQ ID NO:14 (H19102), SEQ ID NO:16 (RSK4), SEQ ID NO:17 (AAD30182), SEQ ID NO:20 (SGK2), SEQ ID NO:22 (PTK9L), SEQ ID NO:26 (AA383293), SEQ ID NO:29 (DRAK2), SEQ ID NO:31 (DRAK1), SEQ ID NO:032 (AA015726), SEQ ID NO:40 (MAK-V), SEQ ID NO:044 (TRAD), SEQ ID NO:044 (TRAD), SEQ ID NO:45 (AA454060), SEQ ID NO:47 (AA234451), SEQ ID NO:48 (AA436054), SEQ ID NO:49 (AA626859), SEQ ID NO:51 (KIAA0904), SEQ ID NO:52 (AA789239), SEQ ID NO:54 (CCRK), SEQ ID NO:55 (CLK4), SEQ ID NO:56 (AA557536), SEQ ID NO:57 (W56561), SEQ ID NO:60 (AA579641), SEQ ID NO:63 (NEK7), SEQ ID NO:66 (CAMKKB), SEQ ID NO:68 (HIPK2), SEQ ID NO:72 (R19609), SEQ ID NO:73 (HRI), SEQ ID NO:78 (AA088547), SEQ ID NO:79 (AA449542), SEQ ID NO:082a (MLK4), SEQ ID NO:82 (MLK4b), SEQ ID NO:84 (RIP4), SEQ ID NO:88 (AA278842), SEQ ID NO:89 (AA195964), SEQ ID NO:90 (MSSK1), SEQ ID NO:93 (TSK4), SEQ ID NO:94 (AI025291), SEQ ID NO:95 (AA948538), SEQ ID NO:96 (AA905446), SEQ ID NO:97 (H85389), SEQ ID NO:100 (AA018361), SEQ ID NO:101 (AA311714), SEQ ID NO:110 (AA452647), SEQ ID NO:111 (AA310219), SEQ ID NO:1 12 (AI086865), SEQ ID NO:114 (MEKK6), and SEQ ID NO:116 (SuRTK106).

### Example 5

### Kinase Assays for Erk, JNK1 and D38 MAP Kinases

293T cells were transiently transfected with HA-p38 or co-transfected with Flag-tagged wt MIX4A, kinase-dead MIX4A, wild-type MLK4B or kinase-dead MLK4B using Lipofectarmine 2000 (Lifetech). Cells were lysed 36 hr post-transfection. Cell lysates normalized to contain equivalent amounts of HA-p38 were immunoprecipitated with anti-HA antibody (Mab HA-11, Babco). Immunoprecipitates were split in two portions, one portion was Western-blotted with anti-HA antibody and the other with a [symbol] o-specific p38 antibody (Promega) to detect activated levels of p38. Activation of [symbol] Jnk1 was measured similarly. (This example applies to AA232253 (SEQ ID NO:82, SEQ ID NO:201).)

Results:

In transient assays wild-type MLK4A and MLK4B (but not kinase-inactive MLK4A(K45M) or MLK4B(K45M)) activate Erk, JNK1 and p38 MAP kinases.

### Example 6

### RAC1 Guanine-Exchange Factor Assay

293T cells were transiently transfected with HA-Rac1 or co-transfected with Flag-tagged Duet C, Duet E, Dbl and HA-Tiam-1. Cells were lysed 36 hour post-transfection. Cell lysates normalized to contain equivalent amounts of Rac1 were affinity precipitated with immobilized GST-PBD (p21-binding domain of Pak3). Bound proteins were Western blotted and probed with anti-HA antibody to detect levels of activated Rac1. ((This example applies to R199772 (Trad/Duet)(SEQ ID NO:44, SEQ ID NO:164).)

Results:

Duet C and Duet E both act as guanine nucleotide exchange factors on Rac1.

### CONCLUSION

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments are exemplary and are not intended as limitations on the scope of the invention. Changes therein and other uses will occur to those skilled in the art which are encompassed within the spirit of the invention are defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed.

In particular, although some formulations described herein have been identified by the excipients added to the formulations, the invention is meant to also cover the final formulation formed by the combination of these excipients. Specifically, the invention includes formulations in which one to all of the added excipients undergo a reaction during formulation and are no longer present in the final formulation, or are present in modified forms.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully describe

Other embodiments are within the following claims.

TABLE 1

| Gene Name | 5P | Prov_Seq_ID_NA | Prov_Seq_ID_AA | Seq_ID_ [symbol] _na | Seq_ID_ [symbol] _aa | Family | Group |
|---|---|---|---|---|---|---|---|
| X69117_h_BARK2_h | H | x | x | 1 | 122 | AGC | GRK |
| AA144574_m BARK2_m | M | 1 | 17 | 2 | 123 | AGC | GRK |
| AA826550_h | H | 140 | 176 | 3 | 124 | AGC | Mo3C11.1_ca |
| AA950957_h | H | 11 | 27 | 4 | 125 | AGC | Mo3C11.1_ca |
| 5R79-45-1_h, TBK1_h | H | 207 | 206 | 5 | 126 | AGC | Mo3C11.1_ca |
| AA305176_h | H | 4, 2 | 18, 20 | 6 | 127 | AGC | NDR |
| AA116841_m | M | 4, 2 | 18, 20 | 7 | 128 | AGC | NDR |
| AA256100_h | H | 3 | 19 | 8 | 129 | AGC | NDR |
| AA210825_h | H | 5 | 21 | 9 | 130 | AGC | PKC |
| AA127299_h | H | 203 | 204 | 10 | 131 | AGC | PKC |
| AA316604_h, EPK2 | H | 6 | 22 | 11 | 132 | AGC | PKC |
| N42050_h PKNbeta | H | [symbol] | 24 | 12 | 133 | AGC | PKC |
| AI021023_m PKNbeta_m | M | x | [symbol] | 13 | 134 | AGC | PKC |
| H19102_h | H | 12 | 28 | 14 | 135 | AGC | S6K |
| AA476563_h RPS6KC1 | H | 9 | 25 | 15 | 136 | AGC | S6K |
| AA626690_h RSK4 | H | 10 | 26 | 16 | 137 | AGC | S6K |
| AA215680_h | H | 227 | 226 | 17 | 138 | AGC | S6K |
| SGK_h | H | x | x | 18 | 139 | AGC | SGK |
| AA107515_m | M | x | x | 19 | 140 | AGC | SGK |
| AA109506_m | M | 13 | 29 | 20 | 141 | AGC | SGK |
| AA557783_h SGK3, SGKL | H | 10 | 32 | 21 | 142 | AGC | SGK |
| R47805_h PTK9L | H | 131 | 167 | 22 | 143 | Atypical | A6 |
| H60215_h | H | 33 | 54 | 23 | 144 | CAMK | AMPK |
| SGK324_h | H | x | x | 24 | 145 | CAMK | CAMK |
| W30246_m SGK324_m | M | 36 | 57 | 25 | 146 | CAMK | CAMK |
| AA383293_h | H | 38 | 59 | 26 | 147 | CAMK | CAMK |
| AA197883_m | M | 34 | 55 | 28 | 148 | CAMK | CAMK |
| AA172300_h DRAK2 | H | 37 | 58 | 29 | 149 | CAMK | DAPK |
| W44150_m DRAK2_m | M | x | x | 30 | 150 | CAMK | DAPK |
| H01248_h, DRAK1_h | H | 40 | 61 | 31 | 151 | CAMK | DAPK |
| AA021445_h | H | 45 | 66 | 32 | 152 | CAMK | EMK |
| 2R22-5-11_h | H | 43 | 64 | 33 | 153 | CAMK | EMK |
| R31237_1_h, AAC33487 | H | x | x | 34 | 154 | CAMK | EMK |
| W90839_m | M | 49 | 70 | 35 | 155 | CAMK | EMK |
| 406785, 5_h | H | x | [symbol] | 36 | 156 | CAMK | EMK |
| AA544838_m 406786_m | M | 48 | 69 | 37 | 157 | CAMK | EMK |
| AA785735_h | H | x | [symbol] | 38 | 158 | CAMK | EMK |
| AA207220_h | H | 46 | 67 | 39 | 159 | CAMK | EMK |
| AA426580_h, MAK_V_h | H | 47 | 66 | 40 | 160 | CAMK | EMK |
| Z36720_h | H | 50 | 71 | 41 | 161 | CAMK | MLCK |
| SGK068_h | H | x | [symbol] | 42 | 162 | CAMK | Trio |
| AA542015_m SGK088_m | M | 39 | 80 | 43 | 163 | CAMK | Trio |
| R19772_h | H | 52 | 73 | 44 | 164 | CAMK | Trio |
| 5R72_8_2_h | H | 53 | 74 | 45 | 165 | CAMK | Unique |
| SGK309_h | H | 159 | 195 | 46 | 166 | CKI | CKI |
| AA234451_h | H | 75 | 75 | 47 | 167 | CKI | CKI |
| AA435956_h | H | 82 | 95 | 48 | 168 | CMGC | CDK |
| AA625859_h | H | 84 | 98 | 49 | 169 | CMGC | CDK |
| AA061797_m | M | x | [symbol] | 50 | 170 | CMGC | CDK |
| AA397553_h CRK7 | H | 61 | 95 | 51 | 171 | CMGC | CDK |
| AA769239_h | H | 85 | 99 | 52 | 172 | CMGC | CDK |
| AA124976_m | M | x | x | 53 | 173 | CMGC | CDK |
| AA575635_m CCRK_m | M | x | x | 54 | 174 | CMGC | CDK |
| AA631990_h CLK4 | H | 105 | 107 | 55 | 175 | CMGC | CLK |
| AA557536_h | H | 69 | 103 | 56 | 176 | CMGC | RCK |
| N28806_h, MOK | H | 90 | 104 | 57 | 177 | CMGC | RCK |
| AB023153_h, ICK | H | [symbol] | x | 58 | 178 | CMGC | RCK |
| AA839940_m | M | [symbol] | x | 59 | 179 | CMGC | RCK |
| AA460132_h | H | 165 | 201 | 60 | 180 | Microbial PK | YGR262_ [symbol] c |
| SGK034_h | H | [symbol] | x | 61 | 181 | Other | C26C2_ce |
| AA103218_m SGK034_m | M | 147 | 183 | 62 | 182 | Other | C26C2_ce |
| NEK7_h, N34132_h | H | 122 | 125 | 63 | 183 | Other | C26C2_ce |
| BCON3_h | H | x | x | 64 | 184 | Other | C26C2_ce |
| AA711629_m | M | 148 | 184 | 65 | 185 | Other | C26C2_ce |
| AA099102_h_CaMKKB | H | 132 | 168 | 66 | 186 | Other | CAMKK |
| 5R69_17_2_h | H | 158 | 194 | 67 | 187 | Other | CTR1 |
| H85811_h | H | 134 | 170 | 68 | 188 | Other | DYRK |
| AA02193_h DYRK3 | H | x | x | 69 | 189 | Other | DYRK |
| AA589241_m DYRK3_m | M | 133 | 169 | 70 | 190 | Other | DYRK |
| 5R72_16_2_h, R19927_h | H | 100 | 112 | 71 | 191 | Other | EIFK |
| R43524_h, HR1_h, R19609 | H | 111 | 114 | 73 | 192 | Other | EIFK |
| 17000057519457_h | H | x | x | 74 | 193 | Other | Endop |
| AA013524_m | M | 135 | 171 | 75 | 194 | Other | Endop |
| 17000139801197_h, IRAKM | H | x | x | 76 | 195 | Other | IRAK |
| AA840598_m IRAKM_m | M | 137 | 173 | 77 | 196 | Other | IRAK |
| AA088547_h | H | 138 | 174 | 78 | 197 | Other | IRE |
| HGP_5644468 | H | x | x | 79 | 198 | Other | KYK2_dd |
| AA449542_m | M | 139 | 175 | 80 | 199 | Other | KYK2_dd |
| 5R57_10_2_m TESK2_m | M | 115 | 116 | 81 | 200 | Other | LIMK |
| AA232253_h | H | 117 | 118 | 82 | 201 | Other | MLK |
| AI375137_h | H | 209 | 210 | 83 | 202 | Other | MLK |
| H97865_h | H | 143 | 179 | 84 | 203 | Other | RIP |
| W20810_m | M | 144 | 180 | 85 | 204 | Other | RIP |
| AA744236_h | H | 211 | 212 | 86 | 205 | Other | SCY1_sc |
| AI052250_h | H | 225 | 226 | 87 | 206 | Other | SCY1_sc |
| AA276842_h | H | 164 | 200 | 88 | 207 | Other | SCY1_sc |
| AA599288_h | H | 145 | 181 | 89 | 208 | Other | SLO87 |
| AA425725_h | H | 148 | 182 | 90 | 209 | Other | SRPK |
| SGK022_1_h | H | x | x | 91 | 210 | Other | STK22A |
| AA050025_m SGK022_m | M | 149 | 185 | 92 | 211 | Other | STK22A |
| AA399669_h | H | 150 | 186 | 93 | 212 | Other | STK22A |
| AA758539_h | H | 151 | 187 | 94 | 213 | Other | STK22A |
| AA853975_h | H | 153 | 189 | 95 | 214 | Other | TSK |
| AA905445_h | H | 222 | 224 | 96 | 215 | Other | TSK |
| H29974_h | H | 58 | 102 | 97 | 216 | Other | UNC |
| AA498104_m H29974_m | M | x | x | 98 | 217 | Other | UNC |
| AA215311_h | H | 155 | 191 | 99 | 218 | Other | UNC |
| AA018361_h | H | 154 | 190 | 100 | 219 | Other | UNC |
| AA311714_h | H | 156 | 192 | 101 | 220 | Other | UNC |
| SGK384_h | H | x | x | 102 | 221 | Other | Unique |
| AA210451_m SGK384_m | M | 106 | 108 | 103 | 222 | Other | Unique |
| SGK071_2_h | H | x | x | 104 | 223 | Other | Unique |
| AA115352_m SGK071_m | M | 161 | 197 | 105 | 224 | Other | Unique |
| O18653.9_h | H | 162 | 198 | 106 | 225 | Other | Unique |
| AA396601_m | M | x | x | 107 | 226 | Other | Unique |
| AA671275_h VRK3 | H | 153 | 199 | 108 | 227 | Other | VRK |
| S71575_m VRK3_m | M | x | x | 109 | 228 | Other | VRK |
| AA452647_h MPSK1 | H | 138 | 172 | 110 | 229 | Other | YPL238_sc |
| H05721_h | H | 100 | 202 | 111 | 230 | Other | YC09_ce |
| AI086865_h | H | 86 | 100 | 112 | 231 | STE | NEK |
| AA638348_h | H | 120 | 124 | 113 | 232 | STE | NEK |
| R86668_h, MKK8 | H | 121 | 125 | 114 | 233 | STE | STE11 |
| PAK6_h 5R95-20-11 | H | 219 | 220 | 115 | 234 | STE | STE20-02 |
| SuRTK108_h 2R41-9-4_h | H | 128, 127 | 129, 130 | 116 | 235 | TK | RTK-20 |
| AA096024_m | M | 128, 127 | 130 | 117 | 236 | TK | RTK-20 |
| SGK2alpha_h | H | x | x | 118 | 237 | AGC | SGK |
| H06950_h_CCRK | H | 67 | 101 | 120 | 238 | CMGC | CDK |
| NM_007170_h TESK2 | H | x | x | 121 | 239 | Other | LIMK |

| Gene Name | Length_NA | Length_AA | ORF_Start | ORF_end | ORF_Length | DNA Repeats | CHR localization |
|---|---|---|---|---|---|---|---|
| X69117_h_BARK2_h | 2067 | 658 | 1 | 2064 | 2064 | x | 22q11 |
| AA144574_m BARK2_m | 1365 | 378 | 2 | 1135 | 1134 | x | NA |
| AA826550_h | 1785 | 419 | 8 | 1264 | 1257 | 285-304 | NA |
| AA950957_h | 3224 | 414 | 55 | 1306 | 1242 | x | 4p16.1 |
| 5R79-45-1_h, TBK1_h | 3013 | 729 | 93 | 2279 | 2167 | x | NA |
| AA305176_h | 1421 | 329 | 53 | 1039 | [symbol] 67 | x | 10p11.2 |
| AA116841_m | 562 | 88 | 3 | 260 | 264 | x | NA |
| AA256100_h | 4983 | 454 | 56 | 1477 | 1392 | x | 12q11 |
| AA210825_h | 3263 | 978 | 117 | 3050 | 2934 | x | 19q13-q13.3 |
| AA127299_h | 315 | 105 | 1 | 315 | 315 | x | NA |
| AA316604_h, EPK2 | 2673 | 890 | 1 | 2670 | 2670 | x | 2p21 |
| N42050_h PKNbeta | 2670 | 889 | 1 | 2667 | 2667 | 2221-2280 | NA |
| AI021023_m PKNbeta_m | 929 | 205 | 2 | 615 | 815 | x | NA |
| H19102_h | 1155 | 384 | 1 | 1152 | 1152 | 599-635 | CHR17 |
| AA476563_h RPS6KC1 | 1410 | 459 | 1 | 1407 | 1407 | x | 12q12-q13.1 |
| AA626690_h RSK4 | 2238 | 745 | 1 | 2235 | 2235 | x | xq21.1 |
| AA215680_h | 1650 | 549 | 1 | 1547 | 1547 | 757-786 | 14q24.3 |
| SGK_h | 1296 | 431 | 1 | 1293 | 1293 | 856-883 | 5q21-q22 |
| AA107515_m | 2432 | 430 | 75 | 1364 | 1290 | 1804-1830 | NA |
| AA109506_m | 1346 | 244 | 2 | 733 | 732 | x | NA |
| AA557783_h SGK3, SGKL | 2250 | 445 | 36 | 1373 | 1338 | x | NA |
| R47805_h PTK9L | 1050 | 349 | 1 | 1047 | 1047 | x | 3p14.3 |
| H60215_h | 2310 | 440 | 420 | 1738 | 1320 | x | 1p31.1-1p32.3 |
| SGK324_h | 3240 | 692 | 7 | 2062 | 2076 | 208-227 | NA |
| W30246_m SGK324_m | 1248 | 297 | 1 | 891 | 591 | x | NA |
| AA383293_h | 2424 | 685 | 1 | 2058 | 2058 | 439-458 | NA |
| AA197883_m | 2421 | 805 | 1 | 2418 | 2418 | x | NA |
| AA172300_h DRAK2 | 1626 | 373 | 262 | 1380 | 1119 | x | 2q31-2q24.3 |
| W44150_m DRAK2_m | 2671 | 372 | 171 | 1285 | 1116 | x | NA |
| H01248_h, DRAK1_h | 1245 | 414 | 1 | 1242 | 1242 | 91-110 | 7p11-q11 |
| AA021445_h | 4321 | 1311 | 146 | 4078 | 3933 | x | 11q22.1-11q22.3 |
| 2R22-5-11_h | 2311 | 436 | 871 | 2178 | 1306 | 81-2251, 1256-12 | NA |
| R31237_1_h, AAC33487 | 2190 | 729 | 1 | 2157 | 2157 | x | NA |
| W90839_m | 1594 | 520 | 1 | 1580 | 1560 | x | NA |
| 406785, 5_h | 4139 | 1330 | 77 | 4085 | 3990 | x | 2q34-q37 |
| AA544838_m 406786_m | 1350 | 230 | 3 | 692 | [symbol] | 1002-1022 | NA |
| AA785735_h | 5183 | 926 | 155 | 2932 | 2776 | x | NA |
| AA207220_h | 2291 | 629 | 103 | 1959 | 1587 | x | NA |
| AA426580_h, MAK_V_h | 2145 | 714 | 1 | 2142 | 2142 | x | 21q11 |
| Z36720_h | 2625 | 874 | 1 | 2622 | 2622 | x | NA |
| SGK068_h | 7710 | 2285 | 1 | 5858 | 8858 | x | NA |
| AA542015_m SGK088_m | 1251 | 127 | 1 | 381 | 381 | x | NA |
| R19772_h | 3854 | 1287 | 1 | 3061 | 3861 | x | 3q13.3-q21 |
| 5R72_8_2_h | 2588 | 514 | 405 | 1947 | 1542 | x | 11p15.1-11p15.2 |
| SGK309_h | 1820 | 506 | 97 | 1620 | 1524 | 843-852 | NA |
| AA234451_h | 2452 | 478 | 405 | 1639 | 1434 | x | NA |
| AA435956_h | 1077 | 266 | 1 | 796 | 795 | x | NA |
| AA625859_h | 911 | 247 | 2 | 742 | 741 | 54-119 | NA |
| AA061797_m | 2615 | 296 | 2 | 689 | 885 | x | NA |
| AA397553_h CRK7 | 4473 | 1490 | 1 | 4470 | 4470 | 1210-1238 | NA |
| AA769239_h | 1 [symbol] | 534 | 6 | 1807 | 1802 | x | 5q23-q23.3 |
| AA124976_m | 1 [symbol] | 337 | 1 | 1011 | 1011 | x | NA |
| AA575635_m CCRK_m | 1380 | 211 | 1 | 633 | 633 | 163-191 | NA |
| AA631990_h CLK4 | 2488 | 499 | 34 | 1530 | 1497 | x | NA |
| AA557536_h | 1831 | 545 | 30 | 1664 | 1635 | 516-538 | NA |
| N28806_h, MOK | 1260 | 419 | 1 | 1257 | 1257 | x | 14q32 |
| AB023153_h, ICK | 1599 | 632 | 1 | 1896 | 1896 | x | NA |
| AA839940_m | 1776 | 413 | 1 | 1239 | 1239 | x | NA |
| AA460132_h | 1428 | 253 | 199 | 957 | 759 | x | 20q12 Amplicon |
| SGK034_h | 3304 | 462 | 1 | 1366 | 1386 | x | NA |
| AA103218_m SGK034_m | 2328 | 251 | 2 | 544 | 843 | x | NA |
| NEK7_h, N34132_h | 7328 | 1952 | 42 | 5897 | [symbol] | x | 12p13.33 |
| BCON3_h | 2164 | 538 | 113 | 1717 | 1608 | 246-267 | NA |
| AA711629_m | 1568 | 378 | 1 | 1134 | 1134 | x | NA |
| AA099102_h_CaMKKB | 1767 | 588 | 1 | 1764 | 1764 | 65-84 | 12q23-q14 |
| 5R69_17_2_h | 3387 | 241 | 1650 | 2572 | 723 | 499-521 | NA |
| H85811_h | 3993 | 1171 | 163 | 3695 | 3513 | 1326-1348 | CHR7 |
| AA02193_h DYRK3 | 2141 | 553 | 253 | 1911 | 1859 | x | NA |
| AA589241_m DYRK3_m | 741 | 168 | 3 | 500 | 504 | x | NA |
| 5R72_16_2_h, R19927_h | 5163 | 1849 | 20 | 4958 | 4947 | x | NA |
| R43524_h, HR1_h, R19609 | 1693 | 530 | 1 | 1890 | 1890 | x | 7p22-p22.3 |
| 17000057519457_h | 3055 | 253 | 219 | 977 | 759 | 2263-2365 | NA |
| AA013524_m | 928 | 218 | 1 | 648 | 648 | x | NA |
| 17000139801197_h, IRAKM | 1791 | 595 | 1 | 1788 | 1758 | x | NA |
| AA840598_m IRAKM_m | 2243 | 392 | 1 | 1175 | 1175 | x | NA |
| AA088547_h | 2759 | 922 | 1 | 2756 | 2766 | x | NA |
| HGP_5644468 | 1857 | 322 | 179 | 1144 | 906 | x | NA |
| AA449542_m | 1251 | 280 | 2 | 641 | 840 | 602-621 | NA |
| 5R57_10_2_m TESK2_140 | 41 | 2 | 124 | 123 | x | NA | m |
| AA232253_h | 2403 | 800 | 1 | 2400 | 2400 | x | NA |
| AI375137_h | 2508 | 835 | 1 | 2505 | 2505 | 2219-2238 | NA |
| H97865_h | 2384 | 834 | 161 | 2062 | 1002 | x | 1q31 |
| W20810_m | 1073 | 289 | 3 | 859 | 567 | x | NA |
| AA744236_h | 2067 | 685 | 1 | 2084 | 2064 | x | 1q23 |
| AI052250_h | 1739 | 505 | 174 | 1635 | 1515 | x | NA |
| AA276842_h | 2658 | 508 | 105 | 2528 | 2424 | 1764-1783 | 11q12-q13 Amplicon |
| AA599288_h | 1949 | 549 | 1 | 1949 | 1949 | x | NA |
| AA425725_h | 1602 | 533 | 1 | 1599 | 1599 | x | Xq28 |
| SGK022_1_h | 1038 | 268 | 154 | 987 | 804 | x | NA |
| AA050025_m SGK022_m | 1004 | 268 | 147 | 950 | 804 | x | NA |
| AA399669_h | 1537 | 292 | 372 | 1244 | 808 | x | 14p11-q11 |
| AA758539_h | 1322 | 358 | 101 | 1174 | 1074 | x | NA |
| AA853975_h | 822 | 273 | 1 | 819 | 819 | x | NA |
| AA905445_h | 1065 | 216 | 365 | 1012 | 645 | x | NA |
| H29974_h | 1638 | 333 | 2 | 1000 | 999 | x | NA |
| AA498104_m H29974_m | 1490 | 412 | 1 | 1238 | 1238 | 701-729 | NA |
| AA215311_h | 2011 | 341 | 199 | 1221 | 1023 | x | NA |
| AA018361_h | 2769 | 481 | 113 | 1555 | 1443 | x | 15q23 |
| AA311714_h | 1875 | 565 | 138 | 1833 | 1595 | x | NA |
| SGK384_h | 117 | 39 | 1 | 117 | 117 | x | NA |
| AA210451_m SGK384_m | 2721 | 349 | 222 | 1268 | 1047 | 2251-2288 | NA |
| SGK071_2_h | 2115 | 704 | 1 | 2112 | 2112 | 57-76, 318-337 | NA |
| AA115352_m SGK071_m | 1729 | 540 | 3 | 1522 | 1820 | x | NA |
| O18653.9_h | 2461 | 540 | 1 | 1620 | 1620 | 240-259 | NA |
| AA396601_m | 1886 | 385 | 3 | 1097 | 1095 | x | NA |
| AA671275_h VRK3 | 1425 | 474 | 1 | 1422 | 1422 | x | 19q13 |
| S71575_m VRK3_m | 1009 | 234 | 3 | 704 | 702 | 937-957 | NA |
| AA452647_h MPSK1 | 915 | 306 | 1 | 915 | 915 | x | 3cen-3q21 |
| H05721_h | 2638 | 581 | 95 | 1537 | 1743 | x | 1p32.3-31 Amplicon |
| AI086865_h | 2463 | 598 | 7 | 2100 | 2094 | x | NA |
| AA638348_h | 2511 | 838 | 1 | 2508 | 2508 | x | NA |
| R86668_h, MKK8 | 3036 | 1011 | 1 | 3033 | 3033 | x | 1p32.3-p31. [symbol] |
| PAK6_h 5R95-20-11 | 2180 | 719 | 1 | 2157 | 2157 | x | 20p12 |
| SuRTK108_h 2R41-9-4_h | 2480 | 495 | 1 | 1485 | 1485 | x | 12p12.3 |
| AA096024_m | 1793 | 153 | 1 | 549 | 549 | 1352-1362 | NA |
| SGK2alpha_h | 1812 | 387 | 88 | 1185 | 1101 | x | NA |
| H06950_h_CCRK | 1359 | 452 | 1 | 1358 | 1358 | x | 9q21.1-q21.3 |
| NM_007170_h TESK2 | 3016 | 555 | 396 | 2060 | 1585 | x | NA |

TABLE 2

| | Patent | Patent | | | | | ID | | | nraa |
|---|---|---|---|---|---|---|---|---|---|---|
| | Seq | Seq | | | nraa | Length | match | % | % | Match |
| SP | ID# na | ID# aa | Family | Group | Pscore | aa | aa | Identity | Similar | ACC# |
| H | 1 | 122 | AGC | GRK | 2.7e−314 | 688 | 687 | 100 | 100 | CAB45857.1 |
| M | 2 | 123 | AGC | GRK | 1.30E−190 | 378 | 371 | 98 | 99 | NP_037029.1 |
| H | 3 | 124 | AGC | o3C11.1_ce | 5.80E−106 | 419 | 262 | 71 | 86 | CAB76471.1 |
| H | 4 | 125 | AGC | o3C11.1_ce | 1.40E−137 | 414 | 414 | 100 | 100 | CAB76471.1 |
| H | 5 | 126 | AGC | o3C11.1_ce | 0 | 729 | 729 | 100 | 100 | \|NP_037386.1 |
| H | 6 | 127 | AGC | NDR | 1.20E−09 | 329 | 73 | 46 | 66 | BAA76817.1 |
| M | 7 | 128 | AGC | NDR | 1.30E−19 | 88 | 42 | 49 | 71 | AAF55594.1 |
| H | 8 | 129 | AGC | NDR | 6.10E−181 | 464 | 463 | 100 | 100 | BAA76809.1 |
| H | 9 | 130 | AGC | PKC | 8.60E−160 | 978 | 615 | 67 | 80 | NP_002733.1 |
| H | 10 | 131 | AGC | PKC | 1.10E−10 | 105 | 42 | 42 | 57 | P05127 |
| H | 11 | 132 | AGC | PKC | 0 | 890 | 590 | 100 | 100 | NP_005804.1 |
| H | 12 | 133 | AGC | PKC | 9.4e−319 | 589 | 589 | 100 | 100 | NP_037487.1 |
| M | 13 | 134 | AGC | PKC | 1.20E−106 | 205 | 204 | 100 | 100 | JC7083 |
| H | 14 | 135 | AGC | S6K | 3.60E−12 | 384 | 94 | 38 | 55 | AAC62495.1 |
| H | 15 | 136 | AGC | S6K | 2.90E−257 | 469 | 469 | 100 | 100 | NP_036556.1 |
| H | 16 | 137 | AGC | S6K | 7.00E−176 | 745 | 745 | 100 | 100 | NP_055311.1 |
| H | 17 | 138 | AGC | S6K | 9.60E−222 | 549 | 649 | 100 | 100 | AAD30182.1 |
| H | 18 | 139 | AGC | SGK | 9.20E−103 | 431 | 430 | 100 | 100 | AAD41091.1 |
| M | 19 | 140 | AGC | SGK | 2.90E−157 | 430 | 426 | 99 | 99 | NP_035491.1 |
| M | 20 | 141 | AGC | SGK | 2.00E−76 | 244 | 244 | 100 | 100 | AAF12757.2 |
| H | 21 | 142 | AGC | SGK | 4.10E−211 | 446 | 375 | 88 | 88 | AAF27051.1 |
| H | 22 | 143 | Atypical | A6 | 5.60E−216 | 349 | 349 | 100 | 100 | NP_009215.1 |
| H | 23 | 144 | CAMK | AMPK | 1.40E−19 | 440 | 68 | 39 | 61 | CAA04119.1 |
| H | 24 | 145 | CAMK | CAMK | 1.50E−165 | 699 | 466 | 65 | 77 | O15075 |
| M | 25 | 146 | CAMK | CAMK | 1.60E−62 | 297 | 199 | 67 | 83 | AAF26675.1 |
| H | 26 | 147 | CAMK | CAMK | 2.60E−48 | 708 | 181 | 44 | 60 | O15075 |
| M | 28 | 148 | CAMK | CAMK | 2.60E−31 | 806 | 147 | 55 | 73 | AAF26675.1 |
| H | 29 | 149 | CAMK | DAPK | 3.10E−121 | 372 | 372 | 100 | 100 | NP_004217.1 |
| M | 30 | 150 | CAMK | DAPK | 7.90E−93 | 372 | 340 | 91 | 95 | NP_004217.1 |
| H | 31 | 151 | CAMK | DAPK | 1.20E−113 | 414 | 414 | 100 | 100 | NP_004751.1 |
| H | 32 | 152 | CAMK | EMK | 5.90E−165 | 1311 | 1053 | 80 | 80 | BAA76843.1 |
| H | 33 | 153 | CAMK | EMK | 1.20E−45 | 436 | 153 | 51 | 70 | T22427 |
| H | 33 | 153 | CAMK | EMK | 1.40E−32 | 436 | 122 | 46 | 65 | AAC15093.1 |
| H | 34 | 154 | CAMK | EMK | 1.30E−184 | 729 | 729 | 100 | 100 | AAC15093.1 |
| M | 35 | 155 | CAMK | EMK | 3.50E−126 | 462 | 462 | 100 | 100 | AAC33487.1 |
| H | 36 | 156 | CAMK | EMK | 0 | 1330 | 1235 | 100 | 100 | BAA09484.1 |
| M | 37 | 157 | CAMK | EMK | 5.10E−59 | 230 | 183 | 79 | 85 | BAA09484.1 |
| H | 38 | 158 | CAMK | EMK | 3.00E−111 | 926 | 636 | 100 | 100 | BAA34501.1 |
| H | 39 | 159 | CAMK | EMK | 7.30E−80 | 629 | 367 | 57 | 69 | NP_055655.1 |
| H | 40 | 160 | CAMK | EMK | 1.40E−244 | 714 | 714 | 100 | 100 | NP_055401.1 |
| H | 41 | 161 | CAMK | MLCK | 8.20E−76 | 874 | 211 | 63 | 80 | AAA73168.1 |
| H | 42 | 162 | CAMK | Trio | 0 | 2286 | 2227 | 100 | 100 | BAA92535.1 |
| M | 43 | 163 | CAMK | Trio | 7.60E−37 | 127 | 67 | 99 | 99 | BAA92535.1 |
| H | 44 | 164 | CAMK | Trio | 0 | 1287 | 1264 | 100 | 100 | NP_008995.1 |
| H | 45 | 165 | CAMK | Unique | 5.00E−20 | 514 | 114 | 41 | 63 | P25323 |
| H | 46 | 166 | CKI | CKI | 3.30E−89 | 508 | 181 | 53 | 65 | AAF59340.1 |
| H | 47 | 167 | CKI | CKI | 8.60E−98 | 478 | 188 | 57 | 68 | AAF59340.1 |
| H | 48 | 168 | CMGC | CDK | 9.60E−39 | 266 | 138 | 62 | 79 | NP_036527.1 |
| H | 49 | 169 | CMGC | CDK | 7.10E−48 | 247 | 146 | 59 | 75 | NP_004187.1 |
| M | 50 | 170 | CMGC | CDK | 2.90E−64 | 296 | 193 | 65 | 78 | NP_004187.1 |
| H | 51 | 171 | CMGC | CDK | 1.10E−264 | 1490 | 1490 | 100 | 100 | AAF36401.1 |
| H | 52 | 172 | CMGC | CDK | 9.20E−101 | 534 | 377 | 82 | 82 | AAF36509.1 |
| M | 53 | 173 | CMGC | CDK | 1.40E−128 | 337 | 225 | 92 | 96 | AAF34871.1 |
| M | 54 | 174 | CMGC | CDK | 3.00E−68 | 211 | 159 | 79 | 84 | NP_036261.1 |
| H | 55 | 175 | CMGC | CLK | 1.50E−242 | 499 | 436 | 91 | 93 | NP_031740.1 |
| H | 56 | 176 | CMGC | RCK | 9.10E−89 | 544 | 343 | 57 | 64 | AAD12719.1 |
| H | 57 | 177 | CMGC | RCK | 2.30E−189 | 419 | 419 | 100 | 100 | NP_055041.1 |
| H | 58 | 178 | CMGC | RCK | 1.50E−180 | 632 | 632 | 100 | 100 | AAF37278.1 |
| M | 59 | 179 | CMGC | RCK | 1.60E−79 | 413 | 198 | 60 | 77 | P20689 |
| H | 60 | 180 | Microbial PK | YGR262_sc | 2.50E−45 | 253 | 102 | 46 | 67 | AAF50799.1 |
| H | 61 | 181 | Other | C26C2_ce | 2.30E−158 | 509 | 258 | 100 | 100 | CAB70864.1 |
| M | 62 | 182 | Other | C26C2_ce | 1.80E−152 | 281 | 243 | 94 | 98 | CAB70864.1 |
| H | 63 | 183 | Other | C26C2_ce | 8.70E−300 | 1952 | 1193 | 99 | 99 | NP_055638.1 |
| H | 64 | 184 | Other | C26C2_ce | 1.10E−254 | 535 | 535 | 100 | 100 | NP_037524.1 |
| M | 65 | 185 | Other | C26C2_ce | 2.50E−208 | 378 | 372 | 98 | 100 | NP_037624.1 |
| H | 66 | 186 | Other | CAMKK | 3.80E−148 | 588 | 588 | 100 | 100 | AAD31507.1 |
| H | 67 | 187 | Other | CTR1 | 9.90E−24 | 287 | 87 | 33 | 52 | JQ1743 |
| H | 68 | 188 | Other | DYRK | 0 | 1171 | 1137 | 97 | 99 | AAD52566.1 |
| H | 69 | 189 | Other | DYRK | 2.10E−280 | 553 | 553 | 100 | 100 | NP_003573.1 |
| M | 70 | 190 | Other | DYRK | 2.30E−95 | 168 | 149 | 90 | 96 | NP_003573.1 |
| H | 71 | 191 | Other | EIFK | 0 | 1649 | 1493 | 90 | 98 | NP_038747.1 |
| H | 73 | 192 | Other | EIFK | 1.50E−220 | 630 | 630 | 100 | 100 | NP_055228.1 |
| H | 74 | 193 | Other | Endop | 2.50E−45 | 253 | 102 | 46 | 67 | AAF50799.1 |
| M | 75 | 194 | Other | Endop | 3.70E−45 | 216 | 100 | 45 | 64 | AAF50799.1 |
| H | 76 | 195 | Other | IRAK | 0 | 596 | 596 | 100 | 100 | NP_009130.1 |
| M | 77 | 196 | Other | IRAK | 1.20E−170 | 392 | 293 | 75 | 85 | NP_009130.1 |
| H | 78 | 197 | Other | IRE | 1.5e−323 | 922 | 746 | 82 | 69 | NP_036146.1 |
| H | 79 | 198 | Other | KYK2_dd | 8.70E−40 | 225 | 102 | 45 | 62 | AAF48758.1 |
| M | 80 | 199 | Other | KYK2_dd | 5.90E−32 | 280 | 109 | 32 | 50 | AAF48758.1 |
| M | 81 | 200 | Other | LIMK | 2.60E−17 | 41 | 37 | 92 | 95 | NP_009101.1 |
| H | 82 | 201 | Other | MLK | 2.50E−282 | 800 | 799 | 100 | 100 | AAF63490.1 |
| H | 83 | 202 | Other | MLK | 8.60E−251 | 835 | 835 | 100 | 100 | AAD29832.1 |
| H | 84 | 203 | Other | RIP | 2.20E−158 | 634 | 365 | 100 | 100 | BAA32317.1 |
| M | 85 | 204 | Other | RIP | 5.30E−158 | 289 | 288 | 100 | 100 | AAF03133.1 |
| H | 86 | 205 | Other | SCY1_sc | 0 | 688 | 688 | 100 | 100 | CAB55300.1 |
| H | 87 | 206 | Other | SCY1_sc | 1.70E−209 | 505 | 354 | 98 | 98 | BAA92598.1 |
| H | 88 | 207 | Other | SCY1_sc | 2.20E−157 | 808 | 396 | 45 | 61 | AAF56933.1 |
| H | 89 | 208 | Other | SLOB? | 7.40E−196 | 649 | 649 | 100 | 100 | BAA91097.1 |
| H | 90 | 209 | Other | SRPK | 5.80E−252 | 533 | 533 | 100 | 100 | NP_0.55185.1 |
| H | 91 | 210 | Other | STK22A | 3.80E−53 | 268 | 122 | 46 | 70 | NP_033461.1 |
| M | 92 | 211 | Other | STK22 | 2.70E−52 | 268 | 127 | 48 | 68 | NP_033462.1 |
| H | 93 | 212 | Other | STK22A | 4.60E−16 | 292 | 112 | 45 | 64 | NP_033461.1 |
| H | 94 | 213 | Other | STK22A | 5.10E−123 | 358 | 322 | 90 | 96 | NP_033462.1 |
| H | 95 | 214 | Other | TSK | 2.10E−33 | 273 | 122 | 46 | 62 | NP_033461.1 |
| H | 96 | 215 | Other | TSK | 2.50E−32 | 216 | 93 | 41 | 58 | NP_033462.1 |
| H | 97 | 216 | Other | UNC | 0.000062 | 333 | 57 | 36 | 56 | AAD32767.1 |
| M | 98 | 217 | Other | UNC | 0.002492 | 412 | 53 | 37 | 52 | BAA77341.1 |
| H | 99 | 218 | Other | UNC | 0.001096 | 341 | 50 | 36 | 56 | BAA77341.1 |
| H | 100 | 219 | Other | UNC | 1.90E−68 | 480 | 247 | 100 | 100 | T17265 |
| H | 101 | 220 | Other | UNC | 1.60E−208 | 585 | 468 | 96 | 96 | BAA91270.1 |
| H | 102 | 221 | Other | Unique | 6.70E−10 | 39 | 27 | 69 | 90 | AAD00575.1 |
| M | 103 | 222 | Other | Unique | 0.000022 | 349 | 38 | 30 | 50 | CAA18116.1 |
| H | 104 | 223 | Other | Unique | 0.000126 | 704 | 54 | 30 | 45 | BAA86576.1 |
| M | 105 | 224 | Other | Unique | 0.007365 | 540 | 25 | 42 | 61 | AAF47916.1 |
| H | 106 | 225 | Other | Unique | 0.31334 | 540 | 52 | 30 | 42 | P10162 |
| M | 107 | 226 | Other | Unique | 0.022948 | 365 | 25 | 34 | 57 | NP_006276.1 |
| H | 108 | 227 | Other | VRK | 3.10E−263 | 474 | 474 | 100 | 100 | BAA90769.1 |
| M | 109 | 228 | Other | VRK | 1.20E−111 | 234 | 191 | 82 | 90 | BAA90769.1 |
| H | 110 | 229 | Other | YPL236_sc | 7.40E−144 | 305 | 304 | 100 | 100 | AAC28337.1 |
| H | 111 | 230 | Other | YQ09_ce | 6.10E−49 | 561 | 135 | 43 | 63 | AAF46188.1 |
| H | 112 | 231 | STE | NEK | 3.30E−30 | 698 | 122 | 48 | 67 | P51954 |
| H | 113 | 232 | STE | NEK | 2.70E−119 | 836 | 357 | 86 | 86 | AAD31939.1 |
| H | 114 | 233 | STE | STE11 | 1.10E−291 | 1011 | 1011 | 100 | 100 | NP_004663.1 |
| H | 115 | 234 | STE | STE20-02 | 7.70E−177 | 719 | 719 | 100 | 100 | BAA94194.1 |
| H | 116 | 235 | TK | RTK-20 | 4.90E−24 | 495 | 77 | 36 | 56 | AAA98465.1 |
| M | 117 | 236 | TK | RTK-20 | 5.30E−18 | 183 | 53 | 39 | 57 | NP_032036.1 |
| H | 118 | 237 | AGC | SGK | 6.30E−112 | 367 | 367 | 100 | 100 | AAF12757.2 |
| H | 120 | 238 | CMGC | CDK | 2.80E−137 | 452 | 452 | 100 | 100 | NP_036251.1 |
| H | 121 | 239 | Other | LIMK | 6.50E−233 | 555 | 555 | 100 | 100 | NP_009101.1 |

| | | Kinase | Kinase | | |
|---|---|---|---|---|---|
| | | Domain(s) | Domain (s) | Profile | Profile |
| SP | Description | start | end | start | end |
| H | BARK2 [Homo sapiens] | 191 | 453 | 1 | 261 |
| M | Adrenergic receptor kinase, beta 2 (G-protein-linked receptor kk [symbol] | 3 | 143 | 121 | 261 |
| H | Serine/threonine protein kinase [Homo sapiens] | 26 | 286 | 1 | 261 |
| H | Serine/threonine protein kinase [Homo sapiens] | 23 | 283 | 1 | 261 |
| H | TANK-binding kinase 1 [Homo sapiens] | 9 | 304 | 1 | 261 |
| H | KIAA0973 protein [Homo sapiens] | 35 | 310 | 1 | 261 |
| M | CG7719 gene product [Drosophila melanogaster] | 24 | 44 | 242 | 261 |
| H | KIAA0965 protein [Homo sapiens] | 90 | 383 | 1 | 261 |
| H | Protein kinase C, mu [Homo sapiens] | 651 | 907 | 1 | 261 |
| H | Protein kinase C, BETA-II TYPE (PKC-BETA-2) [Homo sapiens] | 19 | 24 | 256 | 261 |
| H | Protein kinase C, nu [Homo sapiens] | 576 | 832 | 1 | 261 |
| H | PKNbeta [Homo sapiens] | 559 | 816 | 1 | 261 |
| M | Protein kinase N beta [Homo sapiens] | 1 | 134 | 126 | 261 |
| H | Ribosomal protein S6 kinase 3 [Homo sapiens] | 81 | 333 | 1 | 261 |
| H | Ribosomal protein S6 kinase, 52 kD, polypeptide 1 [Homo sapiens] | 225 | 459 | 1 | 261 |
| H | Ribosomal protein S6 kinase, 90 kD, polypeptide 6 [Homo sapiens] | 73 & 428 | 330 & 883 | 1 | 261 |
| H | Unknown [Homo sapiens] | 153 | 539 | 1 | 261 |
| H | SGK [Homo sapiens] | 98 | 355 | 1 | 261 |
| M | Serum/glucocorticoid regulated kinase [Mus musculus] | 98 | 354 | 1 | 261 |
| M | Protain kinase [Homo sapiens] | 1 | 169 | 24 | 261 |
| H | SGK-like protein SGKL [Homo sapiens] | 182 | 369 | 1 | 261 |
| H | Protein tyrosine kinase 9-like (A6-related protein) [Homo sapiens] | 10 | 17 | 253 | 261 |
| H | Phosphoprotein [Homo sapiens] | 40 | 333 | 1 | 261 |
| H | DCAMKL1 (DOUBLECORTIN-LIKE AND CAM KINASE-LIKE 1) | 368 | 625 | 1 | 261 |
| M | CPG 16 [Mus musculus] | 59 | 297 | 1 | 261 |
| H | DCAMKL1 (DOUBLECORTIN-LIKE AND CAM KINASE-LIKE 1) | 415 | 673 | 1 | 261 |
| M | CPG 16 [Mus musculus] | 514 | 771 | 1 | 261 |
| H | Death-associated protein kinase-related 2 | 33 | 293 | 1 | 261 |
| M | Death-associated protein kinase-related 2 | 32 | 293 | 1 | 261 |
| H | Death-associated protein kinase-related 1 | 61 | 321 | 1 | 261 |
| H | KIAA0999 protein [Homo sapiens] | 8 | 259 | 1 | 261 |
| H | Hypothetical protein F49C.4 - [Caenorhabditis elegans] | 74 | 325 | 1 | 261 |
| H | Cdc25C associated protein kinace C_TAK1 [Homo sapiens] | | | | |
| H | Cdc25C associated protein kinace C_TAK1 [Homo sapiens] | 56 | 307 | 1 | 261 |
| M | R31237_1, partial CDS [Homo sapiens] | 59 | 340 | 1 | 261 |
| H | KIAA0135 gene is related to plm-1 oncogene. [Homo sapiens] | 999 | 1258 | 1 | 261 |
| M | KIAA0135 gene, related to plm-1 oncogene. [Homo sapiens] | 1 | 158 | 23 | 261 |
| H | KIAA0781 protein [Homo sapiens] | 20 | 271 | 1 | 261 |
| H | KIAA0537 gene product [Homo sapiens] | 53 | 304 | 1 | 261 |
| H | Hormonally upregulated neu tumor-associated kinase [Homo sa [symbol] | 61 | 320 | 1 | 261 |
| H | Skeletal muscle myosin light chain kinase [Galus gallus] | 570 | 625 | 1 | 261 |
| H | KIAA1297 [Homo sapiens] | 620 & 1086 | 873 & 1356 | 1 | 261 |
| M | KIAA1297 [Homo sapiens] | 3 | 78 | 186 | 261 |
| H | STK with Dbl- and pleckstrin homology domains [Homo sapiens] | 985 | 1239 | 1 | 261 |
| H | MLCK [Dictyostelium discoideum] | 116 | 381 | 1 | 261 |
| H | CG11533 gene product [Drosophila melanogaster] | 34 | 313 | 1 | 261 |
| H | CG11533 gene product [Drosophila melanogaster] | 21 | 471 | 1 | 261 |
| H | PFTAIRE protein kinase 1 [Homo sapiens] | 1 | 218 | 23 | 261 |
| H | Cyclin-dependent kinase-like 1 (CDC2-related kinase) [Homo sa [symbol] | 1 | 191 | 23 | 261 |
| M | Cyclin-dependent kinase-like 1 (CDC2-related kinase) [Homo sa [symbol] | 1 | 240 | 24 | 261 |
| H | CDC2-related protein kinase 7 [Homo sapiens] | 21 | 1020 | 1 | 261 |
| H | NKIAMRE [Homo sapiens] | 4 | 385 | 1 | 261 |
| M | NKIATRE alpha [Rattus norvegicus] | 1 | 28 | 235 | 261 |
| M | Call cycle related kinase [Homo sapiens] | 1 | 153 | 134 | 261 |
| H | Cyclin-dependent kinase-like 1 (CDC2-related kinase) [Homo sa [symbol] | 177 | 493 | 1 | 261 |
| H | Extracellular signal-regulated kinase 7; ERK7 [Rattus norvegicus | 13 | 305 | 1 | 261 |
| H | Renal tumor antigen [Homo sapiens] | 4 | 285 | 1 | 261 |
| H | Intestinal cell kinase [Homo sapiens] | 4 | 284 | 1 | 261 |
| M | MLCK [Rattus norvegicus] | 109 | 364 | 1 | 261 |
| H | CG10673 gene product [Drosophila melanogaster] | 101 | 187 | 65 | 147 |
| H | Hypothetical protein [Homo sapiens] | 2 | 267 | 1 | 261 |
| M | Hypothetical protein [Homo sapiens] | 59 | 86 | 235 | 261 |
| H | KIAA0344 gene product [Homo sapiens] | 221 | 479 | 1 | 261 |
| H | Nuclear receptor binding protein [Homo sapiens] | 73 | 327 | 1 | 261 |
| M | Nuclear receptor binding protein [Homo sapiens] | 1 | 170 | 85 | 261 |
| H | Ca2+/calmodulin-dependent protein kinase beta [Homo s [symbol] | 165 | 448 | 1 | 261 |
| H | Hypothetical 33.6K protein - rabbit fibroma virus | 24 | 285 | 1 | 261 |
| H | Nuclear body associated kinase 1a [Mus musculus] | 199 | 527 | 1 | 261 |
| H | Dual-specificity tyrosine-(Y)-phosphorylation regulated kinase 3 | 174 | 487 | 1 | 261 |
| M | Dual-specificity tyrosine-(Y)-phosphorylation regulated kinase 3 | 76 | 103 | 235 | 261 |
| H | GCN2 eIF2alpha kinase [Mus musculus] | 280 & 590 | 639 & 1001 | 1 | 261 |
| H | Homo-regulated initiation factor 2-alpha kinase [Homo sapiens] | 167 | 583 | 1 | 261 |
| H | CG10673 gene product [Drosophila melanogaster] | 101 | 187 | 65 | 147 |
| M | (AE003567) CG10673 gene product [Drosophila melanogaster] | 116 | 150 | 116 | 147 |
| H | Interleukin-1 receptor-associated kinase M [Homo sapiens] | 165 | 443 | 1 | 261 |
| M | Interleukin-1 receptor-associated kinase M [Homo sapiens] | 1 | 239 | 19 | 261 |
| H | Ira1, inositol-requiring 1 gene [Mus musculus] | 516 | 777 | 1 | 261 |
| H | CG8173 gene product [Drosophila melanogaster] | 32 | 318 | 1 | 261 |
| M | CG8173 gene product [Drosophila melanogaster] | 12 | 266 | 1 | 261 |
| M | testis-specific kinase 2 [Homo sapiens] | 12 | 39 | 101 | 128 |
| H | Mixed lineage kinase [Homo sapiens] | 16 | 259 | 1 | 261 |
| H | Putative protein-tyrosine kinase [Homo sapiens] | 463 | 723 | 1 | 261 |
| H | KIAA0472 protein [Homo sapiens] | 357 | 620 | 1 | 261 |
| M | Receptor interacting protein 3 [Mus musculus] | 7 | 27 | 161 | 202 |
| H | Hypothetical protein [Homo sapiens] | 57 | 63 | 50 | 78 |
| H | KIAA1360 protein [Homo sapiens] | 32 | 327 | 1 | 261 |
| H | CG1973 gene product [Drosophila melanogaster] | 65 | 131 | 47 | 116 |
| H | Unnamed protein product [Homo sapiens] | 230 | 305 | 81 | 143 |
| H | Serine/threonine kinase 23 [Homo sapiens] | 79 | 531 | 1 | 261 |
| H | Serine/threonine kinase 22A (spermiogenesis associated) [Mus [symbol] | 10 | 265 | 1 | 261 |
| M | Serine/threonine kinase 22B (spermiogenesis associated) | 10 | 265 | 1 | 261 |
| H | Serine/threonine kinase 22A (spermiogenesis associated) | 25 | 280 | 1 | 261 |
| H | Serine/threonine kinase 22B (spermiogenesis associated) [Mus [symbol] | 12 | 272 | 1 | 261 |
| H | Serine/threonine kinase 22A (spermiogenesis associated) | 12 | 267 | 1 | 261 |
| H | Serine/threonine kinase 22B (spermiogenesis associated) | 1 | 213 | 7 | 261 |
| H | Putative protein kinase [Arabidopsis thaliana] | 1 | 329 | 1 | 261 |
| M | UNC-51-like kinase (ULK) 2 [Mus musculus] | 80 | 408 | 1 | 261 |
| H | UNC-51-like kinase (ULK) 2 [Mus musculus] | 8 | 340 | 1 | 261 |
| H | Hypothetical protein DKFZp434C131.1 - human (fragment) | 57 | 313 | 1 | 261 |
| H | Unnamed protein product [Homo sapiens] | 4 | 265 | 1 | 261 |
| H | Serum-Inducible kinase [Homo sapiens] | 1 | 39 | 84 | 124 |
| M | Serine/threonine protein kinase like protein [Arabidopsis thaliane | 80 | 159 | 1 | 86 |
| H | KIAA1264 protein [Homo sapiens] | 1 | 246 | 25 | 261 |
| M | Tie gene product [Drosphila melanogaster] | 9 | 104 | 168 | 261 |
| H | SALIVARY PROLINE-RICH PROTEIN PO(ALLELE K)[HOME [symbol] | 1 | 272 | 16 | 73 |
| M | testis-specific kinase 1 [Homo sapiens] | 68 | 96 | 42 | 71 |
| H | Vaccinia related kinase 3 [Homo sapiens] | 247 | 318 | 63 | 136 |
| M | (AB031052) vaccinia related kinase 3 [Homo sapiens] | 7 | 78 | 63 | 136 |
| H | MPSK [Homo sapiens] | 20 | 290 | 1 | 261 |
| H | CG4523 gene product [Drosphila melanogaster] | 156 | 507 | 1 | 261 |
| H | NEK1 (NIMA-RELATED PROTEIN KINASE 1) [Mus musculus] | 4 | 251 | 1 | 261 |
| H | (AC007055) unknown [Homo sapiens] | 52 | 305 | 1 | 261 |
| H | mitogen-activated protein kinase kinase kinase 6 [Homo sapiens] | 376 | 629 | 6 | 261 |
| H | (AB040812) protein kinase PAK5 [Homo sapiens] | 449 | 700 | 1 | 261 |
| H | (U40827) protein lyrosine kinase [Mus musculus] | 167 | 453 | 1 | 261 |
| M | fibroblast growth factor receptor 3 [Mus musculus] | 8 | 143 | 123 | 261 |
| H | SGK2alpha protein kinase [Homo sapiens] | 35 | 292 | 1 | 261 |
| H | Cell cycle related kinase [Homo sapiens] | 4 | 267 | 1 | 261 |
| H | Testis-specific kinase 2 [Homo sapiens] | 62 | 293 | 5 | 261 |

TABLE 3

| T [symbol] | T [symbol] | 3 [symbol] | T [symbol] | T [symbol] | [symbol] | E [symbol] | [symbol] 3 | [symbol] | [symbol] | [symbol] | [symbol] | [symbol] | [symbol] | [symbol] | [symbol] | [symbol] |
|---|---|---|---|---|---|---|---|---|---|---|---|---|---|---|---|---|
| [symbol] -h | | 1 | | | | | | [symbol] | 137 | [symbol] 721 | [symbol] | 24705 | [symbol] | [symbol] | [symbol] | 13163 |
| [symbol] -h | | 2 | | | | | | 8131 | 32 [symbol] | 12 [symbol] | 114237 | [symbol] 18 | [symbol] | [symbol] | [symbol] | [symbol] |
| [symbol] -h | | 3 | | | | | | [symbol] | [symbol] | 3477 [symbol] | [symbol] 267 | 32237 | 33 [symbol] | [symbol] | [symbol] | [symbol] |
| [symbol] -h | | 4 | | | | | | 984 | 276 | 105547 | 42231 | 4318 | 1234 | [symbol] | [symbol] | 2293 |
| [symbol] -h | | 5 | | | | | | [symbol] | [symbol] 23 | 239783 | [symbol] 23 | [symbol] | [symbol] | 7734 | [symbol] | [symbol] |
| [symbol] -h | | 6 | | | | | | [symbol] 73 | 2674 | [symbol] | [symbol] | [symbol] | [symbol] | 11 [symbol] | 71 [symbol] | 12 [symbol] |
| [symbol] -h | | 7 | | | | | | 8358 | [symbol] | [symbol] 23 | [symbol] | [symbol] | [symbol] | [symbol] | [symbol] | [symbol] |
| [symbol] -h | | 8 | | | | | | 8542 | [symbol] | [symbol] | [symbol] | [symbol] 73 | 11 [symbol] | 29 [symbol] | 83 [symbol] | 12 [symbol] |
| [symbol] -h | | 9 | | | | | | 15 [symbol] | [symbol] | [symbol] | [symbol] | [symbol] | [symbol] | [symbol] 81 | [symbol] | [symbol] |
| [symbol] -h | | 10 | | | | | | 6783 | 13 [symbol] | 12 [symbol] | [symbol] | [symbol] | [symbol] | [symbol] | 80242 | [symbol] |
| [symbol] -h | | 11 | | | | | | 4797 | [symbol] | 837527 | [symbol] | [symbol] | 31171 | [symbol] | [symbol] | [symbol] |
| [symbol] -h | | 12 | | | | | | 3477 | 1794 | [symbol] | [symbol] | [symbol] | [symbol] | [symbol] | [symbol] 27 | [symbol] |
| [symbol] -h | | 13 | | | | | | 3397 | [symbol] | [symbol] 11 | [symbol] | 24129 | [symbol] | [symbol] | [symbol] 27 | [symbol] |
| [symbol] -h | | 14 | | | | | | 2725 | [symbol] | 429781 | 37 [symbol] | 277 [symbol] | 11433 | [symbol] | [symbol] | [symbol] |
| [symbol] -h | | 15 | | | | | | [symbol] | [symbol] | [symbol] | [symbol] | [symbol] | 31811 | [symbol] | [symbol] | [symbol] |
| [symbol] -h | | 16 | | | | | | 8493 | [symbol] | [symbol] | [symbol] | 12319 | [symbol] | [symbol] | [symbol] | [symbol] |
| [symbol] -h | | 17 | | | | | | 3912 | 1915 | [symbol] | [symbol] | [symbol] | [symbol] | 11717 | [symbol] | [symbol] |
| [symbol] -h | | 18 | | | | | | [symbol] | [symbol] | [symbol] 318 | [symbol] | [symbol] | 11720 | 12229 | [symbol] | [symbol] |
| [symbol] -h | | 19 | | | | | | 2439 | [symbol] | [symbol] | [symbol] | [symbol] | 7829 | [symbol] | [symbol] | [symbol] |
| [symbol] -h | | 20 | | | | | | 5184 | [symbol] | [symbol] | [symbol] | [symbol] | [symbol] | [symbol] | [symbol] | [symbol] |
| [symbol] -h | | 21 | | | | | | 2783 | 4479 | [symbol] | [symbol] | [symbol] | 11589 | [symbol] | [symbol] | [symbol] |
| [symbol] -h | | 22 | | | | | | [symbol] | [symbol] | [symbol] | 49111 | [symbol] | [symbol] | [symbol] | [symbol] | [symbol] |
| [symbol] -h | | 23 | | | | | | 4121 | 1921 | [symbol] | 25413 | 25247 | 10839 | 4418 | [symbol] | 7084 |
| [symbol] -h | | 24 | | | | | | [symbol] | 419 | [symbol] | [symbol] | [symbol] | 3479 | [symbol] | [symbol] | [symbol] |
| [symbol] -h | | 25 | | | | | | 4642 | [symbol] | [symbol] | [symbol] 41 | [symbol] | 117943 | [symbol] | [symbol] | [symbol] |
| [symbol] -h | | 27 | | | | | | [symbol] | [symbol] | [symbol] | [symbol] | [symbol] | [symbol] | 21139 | [symbol] | [symbol] |
| [symbol] LC | | 28 | | | | 74 | | [symbol] | 0 | [symbol] | 34171 | [symbol] | [symbol] | [symbol] | [symbol] | [symbol] |
| [symbol] -h | | 29 | | | | | | 2479 | 1073 | 194311 | [symbol] | [symbol] | [symbol] | [symbol] | [symbol] | [symbol] |
| [symbol] LC | | 30 | | | | 29 | | 24571 | 1923 | [symbol] | [symbol] | 3237 | [symbol] | [symbol] | 42340 | [symbol] |
| [symbol] -h | | 31 | | | | | | 8274 | 3082 | [symbol] | [symbol] | [symbol] | [symbol] | [symbol] | [symbol] | [symbol] |
| [symbol] EC | | 32 | | | | | | 7791 | [symbol] | [symbol] | [symbol] | [symbol] | 4775 | 11713 | [symbol] | [symbol] |
| [symbol] -h | | 33 | | | | | | [symbol] | [symbol] | [symbol] | [symbol] | [symbol] | 34729 | [symbol] | [symbol] | [symbol] |
| HCAEC | | 34 | | | | | | [symbol] | 0 | [symbol] | [symbol] | [symbol] | [symbol] | [symbol] | [symbol] | [symbol] |
| [symbol] -h | | 35 | | | | | | [symbol] | 913 | 41543 | [symbol] | [symbol] | 8149 | [symbol] | [symbol] | [symbol] |
| [symbol] -h | | 36 | | | | | | 8123 | 731 | 47839 | 19781 | 20729 | 9429 | [symbol] | [symbol] | [symbol] |
| [symbol] -h | | 37 | | | | | | [symbol] | 77 | 0 | [symbol] | 9411 | [symbol] | [symbol] | [symbol] | [symbol] |
| [symbol] -h | | 38 | | | | | | [symbol] | 2171 | [symbol] | [symbol] | [symbol] | [symbol] | 2447 | 34121 | [symbol] |
| [symbol] -h | | 39 | | | | | | [symbol] | 2472 | [symbol] | [symbol] | [symbol] | 2515 | [symbol] | [symbol] | [symbol] |
| [symbol] -h | | 40 | | | | | | [symbol] | 2472 | [symbol] | [symbol] | [symbol] | [symbol] | 1592 | 79722 | [symbol] |
| [symbol] -h | | 41 | | | | | | 2979 | 0 | [symbol] | [symbol] | [symbol] | [symbol] | [symbol] | 42181 | [symbol] |
| [symbol] -h | | 42 | | | | | | [symbol] | [symbol] | 21473 | [symbol] | 18311 | 13419 | [symbol] | [symbol] | [symbol] |
| [symbol] -h | | 43 | | | | | | [symbol] | 0 | 9314 | 11344 | 12177 | 4129 | 2283 | [symbol] | [symbol] |
| [symbol] -h | | 44 | | | | | | [symbol] | [symbol] | [symbol] | [symbol] | [symbol] | 17833 | [symbol] | [symbol] | [symbol] |
| WT2 [symbol] | | | | | 243 | | | 115 | 357 | 9191 | [symbol] | [symbol] | [symbol] | 134 | [symbol] | [symbol] |
| WT2 [symbol] | | | | | 343 | | | 0 | 427 | 9712 | [symbol] | 0 | 144 | [symbol] | [symbol] | [symbol] |
| WT1 [symbol] | | | | | 391 | | | 0 | 15 | 26479 | 2724 | 725 | [symbol] | [symbol] | [symbol] | [symbol] |
| [symbol] -13 | | | | | 356 | 354 | | 23 | 1684 | 543114 | [symbol] | [symbol] | [symbol] | [symbol] | [symbol] | [symbol] |
| [symbol] -12 | | | | | 354 | 354 | | 11259 | 1283 | [symbol] | [symbol] | 9438 | [symbol] | 2467 | [symbol] | [symbol] |
| [symbol] -h | | | | | 284 | | | 0 | 242 | [symbol] | [symbol] | 1746 | [symbol] | [symbol] | [symbol] | [symbol] |
| [symbol] -h | | | | | 342 | | | 0 | 543 | 20541 | [symbol] | 0 | 2454 | 2223 | [symbol] | [symbol] |
| R*TEC | | | | | 334 | 334 | | 1977 | 0 | [symbol] | 24733 | [symbol] | 323 | 6057 | [symbol] | [symbol] |
| [symbol] -h | | | | | 322 | | | 704 | 0 | 1241 | 7347 | 7053 | [symbol] | 1180 | [symbol] | [symbol] |
| [symbol] | | | | | 330 | | | 905 | 0 | 9314 | 2447 | 1944 | 0 | 140 | [symbol] | [symbol] |
| [symbol] -h | | | | | 328 | | | 7120 | 318 | 191277 | 49029 | 19433 | [symbol] | 2972 | [symbol] | [symbol] |
| HT3 [symbol] | | | | | 327 | | | [symbol] | 0 | [symbol] | 8223 | 320 | [symbol] | 832 | [symbol] | [symbol] |
| [symbol] -h | | | | | 324 | | | 6734 | 80 | [symbol] | [symbol] | 10538 | 7917 | [symbol] | [symbol] | [symbol] |
| HT1 [symbol] | | | | | 321 | | | 187 | 61 | [symbol] | 2927 | 0 | 139 | 1743 | [symbol] | [symbol] |
| HE [symbol] | | | | | [symbol] | | | 3252 | 0 | [symbol] | 12132 | [symbol] | 4353 | 2348 | 61918 | [symbol] |
| [symbol] -h | | | | | 318 | | | 4313 | [symbol] | [symbol] | [symbol] | [symbol] | 31221 | [symbol] | [symbol] | [symbol] |
| [symbol] -h | | | | | 316 | | | 4443 | 254 | [symbol] | [symbol] | [symbol] | [symbol] | 4790 | [symbol] | [symbol] |
| [symbol] -h | | | | | 334 | | | 3520 | 841 | [symbol] | [symbol] | [symbol] | [symbol] | [symbol] | [symbol] | [symbol] |
| [symbol] -h | | | | | 311 | | | [symbol] | 447 | [symbol] | [symbol] | [symbol] | [symbol] | [symbol] | [symbol] | 11249 |
| [symbol] -h | | | | | 309 | | | 45 | [symbol] | [symbol] | [symbol] | 1434 | [symbol] | [symbol] | [symbol] | [symbol] |
| [symbol] -h | | | | | 307 | | | 1276 | 0 | [symbol] | [symbol] | [symbol] | [symbol] | [symbol] | [symbol] | [symbol] |
| [symbol] -h | | | | | 393 | | | 183 | 907 | [symbol] | [symbol] | [symbol] | [symbol] | [symbol] | 44917 | [symbol] |
| [symbol] -h | | | | | 303 | | | 1134 | 700 | [symbol] | 24295 | 8124 | [symbol] | [symbol] | [symbol] | [symbol] |
| [symbol] -h | | | | | 292 | | | [symbol] | 0 | [symbol] | [symbol] | [symbol] | [symbol] | [symbol] | [symbol] | [symbol] |
| [symbol] -h | | | | | 294 | | | 4247 | 11127 | [symbol] | [symbol] | [symbol] | [symbol] | [symbol] | [symbol] | [symbol] |
| [symbol] -h | | | | | 287 | | | 2938 | 1244 | [symbol] | [symbol] | [symbol] | [symbol] | [symbol] | [symbol] | [symbol] |
| [symbol] -h | | | | | 298 | | | 94 | 1547 | [symbol] | [symbol] | 9794 | [symbol] | [symbol] | [symbol] | [symbol] |
| [symbol] -h | | | | | 234 | | | 2252 | [symbol] | [symbol] | 25447 | 14737 | [symbol] | 3701 | [symbol] | [symbol] |
| [symbol] -h | | | | | 282 | | | 195 | 115 | [symbol] | 12005 | 3217 | [symbol] | [symbol] | [symbol] | [symbol] |
| [symbol] -h | | | | | 299 | | | 1375 | 1452 | 417579 | 27541 | 12419 | [symbol] | [symbol] | [symbol] | [symbol] |
| [symbol] -h | | | | | 273 | | | 0 | [symbol] | 34547 | 2711 | [symbol] | 2421 | [symbol] | [symbol] | [symbol] |
| [symbol] -h | | | | | 277 | | | 0 | 2174 | [symbol] | 4719 | 2539 | [symbol] | [symbol] | [symbol] | [symbol] |
| HPALC | | | | | 275 | 276 | | 991 | 123 | 4987 | [symbol] | [symbol] | 532 | 1082 | [symbol] | 7713 |
| HT2 [symbol] | | | | | [symbol] | | | 0 | 59 | [symbol] | 4487 | 0 | [symbol] | [symbol] | [symbol] | [symbol] |
| HT2 [symbol] | | | | | [symbol] | | | 253 | [symbol] | [symbol] | [symbol] | [symbol] | [symbol] | [symbol] | [symbol] | [symbol] |
| [symbol] -11 | | | | | 230 | 239 | | 1719 | 4284 | [symbol] | [symbol] | [symbol] | [symbol] | [symbol] | [symbol] | [symbol] |
| [symbol] - | | | | | 224 | 231 | | 41 | [symbol] | [symbol] | 8231 | [symbol] | [symbol] | [symbol] | [symbol] | [symbol] |
| HT373- [symbol] | | | | | 234 | | | 757 | 0 | [symbol] | [symbol] | [symbol] | 8133 | [symbol] | [symbol] | [symbol] |
| [symbol] -7 | | | | | 233 | 233 | | 0 | [symbol] | [symbol] | 7274 | 2074 | [symbol] | [symbol] | [symbol] | [symbol] |
| [symbol] -4 | | | | | 231 | 231 | | 291 | 2454 | 49149 | 7943 | 8703 | 8477 | 7340 | [symbol] | [symbol] |
| [symbol] -2 | | | | | 229 | 229 | | [symbol] | [symbol] | 74913 | [symbol] | [symbol] | 7022 | 3423 | [symbol] | [symbol] |
| [symbol] -1 | | | | | 227 | 227 | | [symbol] | 4934 | [symbol] | [symbol] | 11203 | 4547 | [symbol] | [symbol] | [symbol] |
| [symbol] -h | | | | | 222 | | | 328 | 459 | 5047 | [symbol] | [symbol] | 3423 | 171 | [symbol] | [symbol] |
| [symbol] -h | | | | | 218 | | | 942 | 0 | [symbol] | [symbol] | [symbol] | [symbol] | [symbol] | [symbol] | [symbol] |
| [symbol] -h | | | | | 214 | | | [symbol] | 1512 | [symbol] | [symbol] | 3792 | [symbol] | 17311 | [symbol] | [symbol] |
| [symbol] -h | | | | | 212 | | | 854 | 647 | [symbol] | [symbol] | [symbol] | [symbol] | [symbol] | [symbol] | 6130 |
| [symbol] -h | | | | | 212 | | | 522 | 1972 | [symbol] | 5781 | 2184 | [symbol] | 1232 | [symbol] | [symbol] |
| HCAEC | | | | | 211 | 211 | | 143 | 0 | 8183 | [symbol] | 184 | [symbol] | 1143 | 24737 | [symbol] |
| [symbol] -h | | | | | 210 | | | [symbol] | 3127 | [symbol] | [symbol] | [symbol] | [symbol] | [symbol] | [symbol] | [symbol] |
| HMEC | | | | | 209 | | | [symbol] | [symbol] | 8034 | 8400 | 0 | 214 | [symbol] | [symbol] | [symbol] |
| [symbol] -h | | | | | 206 | | | 1 | 0 | [symbol] | [symbol] | [symbol] | 1141 | [symbol] | [symbol] | [symbol] |
| [symbol] -h | | | | | 203 | | | 423 | 16 | [symbol] | 1437 | 3994 | 4309 | 991 | [symbol] | [symbol] |
| [symbol] -h | | | | | 291 | | | 415 | [symbol] | [symbol] | [symbol] | [symbol] | [symbol] | [symbol] | [symbol] | [symbol] |
| [symbol] -h | | | | | 183 | | | 130 | 134 | 1142 | [symbol] | 2327 | [symbol] | 247 | [symbol] | [symbol] |
| [symbol] -h | | | | | 187 | | | 438 | 16 | [symbol] | 4851 | 2304 | 1218 | 742 | [symbol] | 4117 |
| [symbol] | | | | | 196 | | | 82 | 0 | 4377 | [symbol] | [symbol] | 1231 | 374 | 21318 | [symbol] |
| [symbol] -h | | | | | 193 | | | [symbol] | 0 | [symbol] | [symbol] | [symbol] | [symbol] | [symbol] | [symbol] | [symbol] |
| [symbol] | | | | | 174 | | | 0 | 0 | [symbol] | 2183 | 0 | [symbol] | 0 | 21244 | 1771 |
| [symbol] -h | | | | | 91 | | | 314 | 273 | [symbol] | [symbol] | [symbol] | 4234 | [symbol] | [symbol] | [symbol] |
| [symbol] -h | | | | | [symbol] | | | 737 | [symbol] | [symbol] | [symbol] | [symbol] | [symbol] | [symbol] | [symbol] | [symbol] |
| [symbol] -h | | | | | 57 | | | 0 | 234 | [symbol] | [symbol] | 2152 | [symbol] | 4 | [symbol] | [symbol] |
| [symbol] -h | | | | | 54 | | | 0 | 1134 | [symbol] | [symbol] | 3702 | 4377 | 442 | 37903 | [symbol] |
| [symbol] -h | | | | | 53 | | | 0 | 1323 | [symbol] | [symbol] | 732 | 1175 | [symbol] | [symbol] | [symbol] |
| [symbol] -h | | | | | 51 | | | 277 | 841 | [symbol] | [symbol] | [symbol] | [symbol] | [symbol] | [symbol] | 4737 |
| [symbol] -h | | | | | [symbol] | | | 1379 | 744 | [symbol] | [symbol] | 2313 | [symbol] | 1472 | [symbol] | [symbol] |
| HELA- [symbol] -031 [symbol] | | | | 79 | | | | [symbol] | 2343 | [symbol] | 4142 | [symbol] | [symbol] | [symbol] | [symbol] | [symbol] |
| HELA- [symbol] -031 [symbol] | | | | 81 | | | | [symbol] | [symbol] | [symbol] | 3757 | [symbol] | 4922 | [symbol] | [symbol] | [symbol] |
| HELA- [symbol] -031 [symbol] | | | | 82 | | | | 935 | [symbol] | [symbol] | 3429 | [symbol] | [symbol] | [symbol] | [symbol] | [symbol] |
| HELA- [symbol] -031 [symbol] | | | | 83 | | | | 944 | [symbol] | [symbol] | [symbol] | [symbol] | [symbol] | [symbol] | [symbol] | [symbol] |
| HELA- [symbol] -031 [symbol] | | | | [symbol] | | | | 1271 | [symbol] | [symbol] | [symbol] | 4751 | [symbol] | [symbol] | [symbol] | [symbol] |
| HELA- [symbol] -031 [symbol] | | | | [symbol] 3 | | | | 323 | 416 | [symbol] | [symbol] | [symbol] | [symbol] | [symbol] | [symbol] | [symbol] |
| HELA-10h-031 [symbol] | | | | [symbol] 2 | | | | 419 | [symbol] | [symbol] | [symbol] | [symbol] | [symbol] | 5137 | [symbol] | [symbol] |
| HELA-11h-031 [symbol] | | | | [symbol] 4 | | | | 0 | 0 | [symbol] | [symbol] | 312 | [symbol] | [symbol] | [symbol] | [symbol] |
| HELA-12h-031 [symbol] | | | | [symbol] | | | | 0 | [symbol] | [symbol] | 3207 | [symbol] | [symbol] | [symbol] | [symbol] | [symbol] |
| MC1- [symbol] | | | | 148 | | | | 1454 | 244 | [symbol] | [symbol] | [symbol] | [symbol] | [symbol] | [symbol] | [symbol] |
| MC2- [symbol] | | | | 144 | | | | 1834 | 164 | [symbol] | [symbol] | [symbol] | [symbol] | [symbol] | [symbol] | [symbol] |
| MC3- [symbol] | | | | 150 | | | | 913 | 254 | [symbol] | [symbol] | [symbol] | [symbol] | [symbol] | [symbol] | [symbol] |
| [symbol] -19 | | | | 152 | | | | 1113 | 0 | [symbol] | 1124 | [symbol] | [symbol] | 0 | [symbol] | [symbol] |
| [symbol] -75 | | | | 154 | | | | 1194 | 0 | [symbol] | 3794 | [symbol] | [symbol] | [symbol] | [symbol] | [symbol] |
| [symbol] -288 | | | | 156 | | | | 231 | 0 | [symbol] | [symbol] | [symbol] | [symbol] | 447 | [symbol] | [symbol] |
| [symbol] -295 | | | | 158 | | | | 0 | 342 | [symbol] | [symbol] | [symbol] | 892 | 378 | [symbol] | [symbol] |
| CCRY-CRM | | | | 160 | | | | 1991 | 274 | [symbol] | 317 | [symbol] | [symbol] | 418 | [symbol] | [symbol] |
| DU-145 | | | | 162 | | | | 209 | 425 | 7542 | [symbol] | [symbol] | [symbol] | 118 | [symbol] | [symbol] |
| MCT 115 | | | | 154 | | | | 121 | 0 | 21002 | [symbol] | [symbol] | [symbol] | [symbol] | [symbol] | 1953 |
| mo SAIT | 156 | | | | | | | 13873 | 0 | [symbol] 24 | [symbol] 41 | 16731 | 12712 | [symbol] | [symbol] | 6334 |
| mo SAVT | 153 | | | | | | | 13427 | 1232 | 37438 | [symbol] | 8797 | 5704 | [symbol] | [symbol] | 51 [symbol] |
| MCP [symbol] | 151 | | | | | | | 15337 | 1085 | [symbol] 7 | 12914 | 21779 | 1 [symbol] | 10792 | 87 [symbol] 0 | 9415 |
| M14 | 143 | | | | | | | 8482 | 145 | 2 [symbol] 14 | 17174 | 16734 | 183 [symbol] 4 | 2844 | 63303 | 4 [symbol] 20 |
| UACC-257 | 142 | | | | | | | 11438 | 0 | 1888 [symbol] | [symbol] 7 | 10437 | 12037 | 41 [symbol] | 2 [symbol] | 2737 |
| UACC-42 | 143 | | | | | | | 3456 | 123 | 14725 | 21877 | 11114 | 27 [symbol] 4 | 1820 | 27441 | 4941 |
| [symbol] MEL-28 | 144 | | | | | | | 4479 | 0 | 390827 | 13781 | 7222 | 2 [symbol] 47 | 2124 | 2 [symbol] 1 | [symbol] 24 |
| UP-31 | 143 | | | | | | | 8347 | 0 | 242414 | 19130 | [symbol] 7 | 23333 | 41 [symbol] | 1 [symbol] 230 | 8343 |
| S [symbol] -MEL-4 | 142 | | | | | | | 5444 | 680 | 43 [symbol] | [symbol] | [symbol] 34 | 3 [symbol] 782 | 70 [symbol] | 2 [symbol] 01 | 12 [symbol] 2 |
| MCM-12 | 141 | | | | | | | 6633 | 377 | 43670 | 12 [symbol] | [symbol] | 4823 | 71 [symbol] | 3 [symbol] 2 | 3132 |
| SOC-MEL-2 | 140 | | | | | | | 29 [symbol] | 331 | 391883 | [symbol] 6 | 13817 | 13 [symbol] 741 | 1004 | 20577 | 5700 |
| MCT- [symbol] | 139 | | | | | | | 1187 | 0 | 362064 | 14372 | [symbol] 230 | 11433 | [symbol] 78 | 14730 | 18341 |
| M [symbol] -2 [symbol] | 138 | | | | | | | [symbol] | 0 | 403464 | 1 [symbol] 20 | [symbol] 70 | 21371 | 5334 | 45000 | 7827 |
| COLO [symbol] | 137 | | | | | | | 6249 | 0 | 384338 | 1 [symbol] | 8560 | 11 [symbol] 1 | 4 [symbol] | 877 [symbol] | [symbol] |
| LO [symbol] | 136 | | | | | | | 4339 | 294 | 1470004 | 237 [symbol] 3 | 18725 | 23414 | [symbol] | 200000 | 1815 |
| S [symbol] -4 [symbol] | 135 | | | | | | | 11829 | 0 | 822854 | 1 [symbol] 64 | [symbol] 62 | [symbol] 4 | 7213 | 3 [symbol] | 1702 |
| TW-10 | 134 | | | | | | | 3634 | 1142 | 232633 | 21183 | 1832 | [symbol] 2 | [symbol] 041 | 4 [symbol] 7 | [symbol] |
| MCT 110 | 133 | | | | | | | 3482 | 0 | 160614 | 13 [symbol] | 73 [symbol] | [symbol] | 40 [symbol] | 37000 | 8317 |
| 70 [symbol] -0 | 132 | | | | | | | 3284 | 0 | 3 [symbol] 64 | 17729 | 4 [symbol] 2 | 13 [symbol] 4 | 2041 | 34171 | [symbol] 7 |
| HCC-23 [symbol] | 131 | | | | | | | [symbol] 70 | 2737 | 3 [symbol] 8 | 179 [symbol] | 17 [symbol] | 1 [symbol] | 3371 | [symbol] | [symbol] |
| AC [symbol] | 130 | | | | | | | 6662 | 117 | 84811 | [symbol] | 10054 | 6438 | [symbol] | 3 [symbol] 0 | 5706 |
| PC-2 | 129 | | | | | | | 13 [symbol] | 0 | 4 [symbol] 24 | 14104 | 4879 | [symbol] 473 | 3508 | 343 [symbol] 2 | [symbol] 3 |
| [symbol] 3 [symbol] | 128 | | | | | | | [symbol] 33 | 278 | 4 [symbol] 33 | [symbol] | 7110 | 13004 | 3324 | 43444 | 1004 |
| OL [symbol] | 127 | | | | | | | 14823 | 134 | 30 [symbol] | 12131 | 3150 | 3 [symbol] 0 | 3261 | 37777 | 3307 |
| C [symbol] -1 | 126 | | | | | | | 3646 | 722 | 71294 [symbol] | 127140 | [symbol] | 13345 | 4217 | 38247 | [symbol] 7 |
| [symbol] | 125 | | | | | | | 35 [symbol] | 257 | 420391 | 13347 | [symbol] 20 | 10111 | 7543 | 378 [symbol] | 4327 |
| AC [symbol] | 124 | | | | | | | 8123 | 0 | 64309 | 140 [symbol] 0 | 41874 | 17 [symbol] | 4314 | [symbol] 104 | 8283 |
| [symbol] | 123 | | | | | | | 2276 | 0 | 372834 | 18300 | 5419 | 297 [symbol] | 1191 | [symbol] 0043 | [symbol] |
| [symbol] N [symbol] C | 122 | | | | | | | 6283 | 217 | 200477 | 17102 | 14334 | 29787 | 67 [symbol] | 32473 | 1267 |
| ML-40 | 121 | | | | | | | 11442 | 0 | 137022 | [symbol] | 63201 | 224 [symbol] 3 | 4184 | 384440 | 4200 |
| MCL T-4 | 120 | | | | | | | 4384 | 0 | 440060 | 127 [symbol] | 10231 | 18 [symbol] | 2 [symbol] 4 | 26300 | 4478 |
| OVCAR-1 | 119 | | | | | | | 3830 | 364 | [symbol] 25 | 14534 | 5452 | 41 [symbol] | 4117 | 25736 | [symbol] 34 |
| X- [symbol] | 118 | | | | | | | 2180 | 794 | 804729 | 14781 | [symbol] 5 | 20028 | 2300 | 54213 | [symbol] 030 |
| OVCAR-4 | 117 | | | | | | | 2854 | 3367 | 43 [symbol] 335 | 10520 | 8040 | 117 [symbol] 7 | [symbol] 1 | 42100 | 2303 |
| CC [symbol] -CDM | 116 | | | | | | | 4370 | 0 | 3 [symbol] 707 | 217 [symbol] 7 | 9518 | 81543 | [symbol] | 38120 | 4575 |
| OVCAR-3 | 115 | | | | | | | 3371 | 9 | 14 [symbol] 64 | 10 [symbol] 74 | 1044 | 21026 | 5119 | 52017 | 5413 |
| S [symbol] -638 | 114 | | | | | | | 4819 | 0 | [symbol] 2870 | 13841 | 4716 | 1 [symbol] 3 | 2834 | 3 [symbol] 5 | 5113 |
| HC [symbol] - [symbol] | 113 | | | | | | | 2174 | 384 | 345970 | 13734 | [symbol] 54 | 11834 | 3200 | 19676 | 4413 |
| SF-295 | 112 | | | | | | | 11905 | 218 | 98183 | 19707 | [symbol] 0 | 7154 | 2946 | 40830 | 1 [symbol] 2 |
| A [symbol] TCC | 111 | | | | | | | 17217 | [symbol] | 376850 | 27212 | 12354 | 1 [symbol] 17 | 6358 | 47430 | 8324 |
| SF-288 | 110 | | | | | | | 3084 | 1353 | 233341 | [symbol] | [symbol] 41 | 11 [symbol] 24 | 2370 | 30243 | 3718 |
| MCAH [symbol] | 109 | | | | | | | 4147 | 0 | 1128384 | 19211 | 19185 | 32502 | 318 [symbol] 3 | 44011 | [symbol] 45 |
| w291 | 108 | | | | | | | 9538 | 373 | 230300 | 107 [symbol] | [symbol] | 120 [symbol] | 11 [symbol] | 54075 | 8200 |
| MC- [symbol] | 107 | | | | | | | 6777 | 0 | 457383 | 13000 | [symbol] | 1 [symbol] 3 | 4430 | 2 [symbol] | 5244 |
| SN [symbol] -75 | 106 | | | | | | | 7578 | 630 | 27880 | 8030 | 323 | 9216 | 2822 | 41104 | 4 [symbol] 18 |
| MC- [symbol] | 105 | | | | | | | 6471 | 821 | 188027 | 19449 | 14479 | 18523 | 2322 | 42032 | 6532 |
| [symbol] M-19 | 104 | | | | | | | 7360 | 0 | 210001 | 17 [symbol] 44 | 7 [symbol] 4 | 12434 | 3100 | 50664 | 1744 |
| MC-M224 | 103 | | | | | | | 32964 | 310 | 106338 | [symbol] 4 | 10726 | 24181 | 3034 | 61587 | [symbol] 3 |
| SM-CM-3 | 102 | | | | | | | 8457 | 0 | 302788 | 16547 | 1470 | 11077 | 2287 | 33178 | 2004 |
| NCH [symbol] 23 | 101 | | | | | | | 5414 | 371 | 108502 | 12284 | 8342 | 15724 | [symbol] | 3 [symbol] | 3418 |
| [symbol] ROV1 | 100 | | | | | | | 13803 | 708 | 313545 | 14137 | [symbol] 515 | [symbol] 679 | 2 [symbol] 41 | 41000 | 3314 |
| [symbol] V [symbol] | 99 | | | | | | | 8228 | 1110 | 3 [symbol] 16 | 10012 | 6432 | 217 [symbol] | 3010 | 25017 | 3824 |
| OVCAR-6 | 98 | | | | | | | 12781 | 814 | 16774 | 10381 | 4170 | [symbol] 4 | 3120 | 31129 | 4019 |
| MCP- [symbol] 2 | 97 | | | | | | | 7808 | 0 | 397 [symbol] | 22816 | 4200 | 3 [symbol] 00 | 3 [symbol] 2 | 33300 | 3000 |
| [symbol] 3831492 [symbol] 12 | 48 | | | | | | | 130 [symbol] | 0 | 177631 | 14183 | [symbol] | [symbol] 047 | 3428 | 3200 | 4127 |
| in [symbol] C 102142 017 | 47 | | | | | | | 4478 | 137 | 8972 | 14410 | 1 [symbol] 0 | 3 [symbol] 4 | 5 [symbol] | 20037 | 3335 |
| in [symbol] 2025792 [symbol] | 44 | | | | | | | 13802 | 230 | 153425 | 321 [symbol] 4 | [symbol] | 14419 | 3871 | 29129 | 1 [symbol] 2 |
| TCCP | 25 | | | | | | | 6808 | 0 | [symbol] 454 | 15245 | 19200 | 19187 | 3430 | 22053 | 8471 |
| ASCS-1 | | | | | | | wt | 1421 | 5421 | 44209 | 11710 | 0 | 0 | 0 | 0 | 0 |
| ASCS-3 | | | | | | | wt | 130 | 4212 | 28334 | 6707 | 0 | 0 | 0 | 0 | 0 |
| ASCS-4 | | | | | | | wt | 278 | 8223 | 38473 | 1 [symbol] | 0 | 0 | 0 | 0 | 0 |
| ASCS-6 | | | | | | | wt | 440 | 2672 | 28000 | 10727 | 0 | 0 | 0 | 0 | 0 |
| ASCS-7 | | | | | | | wt | 148 | 182 | 18733 | 6765 | 0 | 0 | 0 | 0 | 0 |
| EKVX-1 | | | | | | | [symbol] | 844 | 1243 | [symbol] 4 | 16734 | 0 | 0 | 0 | 0 | 0 |
| EKVX-4 | | | | | | | [symbol] | 1480 | 43 [symbol] | 7 [symbol] | 22314 | 0 | 0 | 0 | 0 | 0 |
| EKVX-3 | | | | | | | [symbol] | 836 | 834 | 32907 | 12511 | 0 | 0 | 0 | 0 | 0 |
| EKVX-6 | | | | | | | [symbol] | 0 | 0 | 99902 | 1740 | 0 | 0 | 0 | 0 | 0 |
| EKVX-7 | | | | | | | [symbol] | 732 | 1374 | [symbol] 2 | 11042 | 0 | 0 | 0 | 0 | 0 |
| MCP-7-1 | | | | | | | wt | 240 | 718 | 88119 | 10 [symbol] | 0 | 0 | 0 | 0 | 0 |
| MCP-7-3 | | | | | | | wt | 407 | 6248 | [symbol] 380 | 7 [symbol] 3 | 0 | 0 | 0 | 0 | 0 |
| MCP-7-4 | | | | | | | wt | 438 | 1154 | 140711 | 14017 | 0 | 0 | 0 | 0 | 0 |
| MCP-7-6 | | | | | | | wt | 224 | 1050 | 81148 | 11019 | 0 | 0 | 0 | 0 | 0 |
| MCP-7-7 | | | | | | | wt | 0 | 1337 | 10847 | 8720 | 0 | 0 | 0 | 0 | 0 |
| AD [symbol] -RE [symbol] -1 | | | | | | | [symbol] | 0 | 3883 | 51125 | 1 [symbol] 444 | 0 | 0 | 0 | 0 | 0 |
| AD [symbol] -RE [symbol] -3 | | | | | | | [symbol] | 123 | 823 | 27134 | 8042 | 0 | 0 | 0 | 0 | 0 |
| AD [symbol] -RE [symbol] -4 | | | | | | | [symbol] | 0 | 941 | 15870 | 4440 | 0 | 0 | 0 | 0 | 0 |
| AD [symbol] -RE [symbol] -6 | | | | | | | [symbol] | 204 | 472 | 33538 | 7540 | 0 | 0 | 0 | 0 | 0 |
| AD [symbol] -RE [symbol] -7 | | | | | | | [symbol] | 980 | 7302 | 12023 | 13042 | 0 | 0 | 0 | 0 | 0 |
| WI 3 [symbol] -1 | | | | | | | wt | 819 | 0 | 41827 | 24007 | 0 | 0 | 0 | 0 | 0 |
| WI 3 [symbol] -3 | | | | | | | wt | 508 | 217 | 40570 | 15233 | 0 | 0 | 0 | 0 | 0 |
| WI 3 [symbol] -4 | | | | | | | wt | 642 | 751 | 32719 | 5317 | 0 | 0 | 0 | 0 | 0 |
| WI 3 [symbol] -6 | | | | | | | wt | 1161 | 3721 | 17081 | 11307 | 0 | 0 | 0 | 0 | 0 |
| WI 3 [symbol] -7 | | | | | | | wt | 218 | 4817 | 84181 | 12200 | 0 | 0 | 0 | 0 | 0 |
| H [symbol] L [symbol] -1 | | | | | | | HPY E [symbol] | 523 | 12821 | 18493 | 13319 | 0 | 0 | 0 | 0 | 0 |
| H [symbol] L [symbol] -3 | | | | | | | HPY E [symbol] | 480 | 643 | 51637 | 12033 | 0 | 0 | 0 | 0 | 0 |
| H [symbol] L [symbol] -4 | | | | | | | HPY E [symbol] | 502 | 3311 | 46288 | 7005 | 0 | 0 | 0 | 0 | 0 |
| H [symbol] L [symbol] -6 | | | | | | | HPY E [symbol] | 213 | 1104 | 40234 | 10027 | 0 | 0 | 0 | 0 | 0 |
| H [symbol] L [symbol] -7 | | | | | | | HPY E [symbol] | 0 | 1049 | 37504 | [symbol] | 0 | 0 | 0 | 0 | 0 |
| WI 2 [symbol] -1 | | | | | | | [symbol] | 944 | 0 | 88087 | 14773 | 0 | 0 | 0 | 0 | 0 |
| WI 2 [symbol] -3 | | | | | | | [symbol] | 304 | 0 | 80294 | 14722 | 0 | 0 | 0 | 0 | 0 |
| WI 2 [symbol] -4 | | | | | | | [symbol] | 304 | 2070 | 78198 | 29063 | 0 | 0 | 0 | 0 | 0 |
| WI 2 [symbol] -5 | | | | | | | [symbol] | 204 | 1404 | 33234 | 10427 | 0 | 0 | 0 | 0 | 0 |
| WI 2 [symbol] -7 | | | | | | | [symbol] | 440 | 0 | 41806 | 10304 | 0 | 0 | 0 | 0 | 0 |
| AS [symbol] -2 | | | | | | | wt | 194 | 4270 | 37307 | 1002 | 0 | 0 | 0 | 0 | 0 |
| EKVX-2 | | | | | | | [symbol] | 538 | 2003 | 23382 | 15428 | 0 | 0 | 0 | 0 | 0 |
| MCT-116-1 | | | | | | | wt | 428 | 8497 | 38339 | 9130 | 0 | 0 | 0 | 0 | 0 |
| MCT-116-2 | | | | | | | wt | 1123 | 3794 | 480330 | 21130 | 0 | 0 | 0 | 0 | 0 |
| MT [symbol] -2 | | | | | | | [symbol] | [symbol] 1 | 1380 | 62391 | 13827 | 0 | 0 | 0 | 0 | 0 |
| SP [symbol] -1 | | | | | | | wt | 0 | 1849 | 10007 | 13003 | 0 | 0 | 0 | 0 | 0 |
| SP [symbol] -2 | | | | | | | wt | 112 | 2474 | 46281 | 12141 | 0 | 0 | 0 | 0 | 0 |
| SP-268-1 | | | | | | | [symbol] | 962 | 317 | 42852 | 13334 | 0 | 0 | 0 | 0 | 0 |
| SP-268-2 | | | | | | | [symbol] | 818 | 850 | 74505 | 12530 | 0 | 0 | 0 | 0 | 0 |
| OVCAR- [symbol] -1 | | | | | | | wt | 332 | 98 | 70236 | 13104 | 0 | 0 | 0 | 0 | 0 |
| OVCAR- [symbol] -2 | | | | | | | wt | 342 | 1843 | 101777 | 10427 | 0 | 0 | 0 | 0 | 0 |
| OVCAR- [symbol] -1 | | | | | | | [symbol] | 220 | 941 | 12838 | 14287 | 0 | 0 | 0 | 0 | 0 |
| OVCAR- [symbol] -2 | | | | | | | [symbol] | 10 | 10 | 1110 | 6476 | 0 | 0 | 0 | 0 | 0 |
| MCF-7-2 | | | | | | | wt | 291 | 1423 | 11082 | 7 [symbol] 2 | 0 | 0 | 0 | 0 | 0 |
| AD [symbol] - [symbol] -2 | | | | | | | [symbol] | 0 | 731 | 31787 | [symbol] 442 | 0 | 0 | 0 | 0 | 0 |
| M [symbol] -2 | | | | | | | HPV E4 | 518 | 0 | 112434 | 14374 | 0 | 0 | 0 | 0 | 0 |
| SW [symbol] -1 | | | | | | | [symbol] | 328 | 0 | 54775 | 8 [symbol] | 0 | 0 | 0 | 0 | 0 |
| SW [symbol] -2 | | | | | | | [symbol] | 1061 | 7828 | 88139 | 12351 | 0 | 0 | 0 | 0 | 0 |
| WI2 [symbol] -2 | | | | | | | [symbol] | [symbol] 23 | 2573 | 57384 | 18061 | 0 | 0 | 0 | 0 | 0 |
| C33A-1 | | | | | | | [symbol] | [symbol] 02 | 18707 | 9547 | 18 [symbol] | 0 | 0 | 0 | 0 | 0 |
| C33A-2 | | | | | | | [symbol] | [symbol] 4 | 1154 | 43004 | 11722 | 0 | 0 | 0 | 0 | 0 |
| U2C [symbol] -1 | | | | | | | [symbol] | 110 | 86 | 151805 | 10077 | 0 | 0 | 0 | 0 | 0 |
| U2C [symbol] -2 | | | | | | | [symbol] | 121 | 0 | 140671 | 22177 | 0 | 0 | 0 | 0 | 0 |
| M [symbol] -1 | | | | | | | wt | 287 | 582 | 23 [symbol] 4 | 10011 | 0 | 0 | 0 | 0 | 0 |
| M [symbol] -2 | | | | | | | wt | [symbol] 0 | 985 | 34277 | 11870 | 0 | 0 | 0 | 0 | 0 |
| WI [symbol] -2 | | | | | | | wt | 415 | 2476 | 38744 | 25529 | 0 | 0 | 0 | 0 | 0 |
| M [symbol] -1 | | | | | | | | 574 | 283 | 1294 | 110430 | 0 | 0 | 0 | 0 | 0 |
| M [symbol] -2 | | | | | | | | 1123 | 3287 | 36129 | 1 [symbol] 56 | 0 | 0 | 0 | 0 | 0 |
| M [symbol] -3 | | | | | | | | [symbol] 4 | 894 | 34711 | 31829 | 0 | 0 | 0 | 0 | 0 |
| M [symbol] -4 | | | | | | | | 1168 | 0 | 12352 | 7 [symbol] | 0 | 0 | 0 | 0 | 0 |
| M [symbol] - [symbol] | | | | | | | | 730 | 0 | 8477 | 277577 | 0 | 0 | 0 | 0 | 0 |
| M [symbol] - [symbol] | | | | | | | | 872 | 8439 | 13002 | 31582 | 0 | 0 | 0 | 0 | 0 |
| M [symbol] - [symbol] | | | | | | | | 1350 | 1097 | 14384 | 24700 | 0 | 0 | 0 | 0 | 0 |
| M [symbol] - [symbol] | | | | | | | | 834 | 0 | 15325 | 11034 | 0 | 0 | 0 | 0 | 0 |
| [symbol] -7 | | | | | | | | 4281 | 4123 | [symbol] 781 | [symbol] 7 | 0 | 0 | 0 | 0 | 0 |
| [symbol] -4 | | | | | | | | 1603 | 0 | 1,28,244 | 31,888 | 0 | 0 | 0 | 0 | 0 |
| [symbol] -0 | | | | | | | | 1704 | 362 | 23,137 | 22, [symbol] | 0 | 0 | 0 | 0 | 0 |
| [symbol] -11 | | | | | | | | 2 [symbol] | 2718 | [symbol] | 36,367 | 0 | 0 | 0 | 0 | 0 |
| [symbol] -12 | | | | | | | | 1421 | 0 | 14,3018 | 14,764 | 0 | 0 | 0 | 0 | 0 |
| [symbol] -10 | | | | | | | | 1137 | 11437 | [symbol] ,778 | 2, [symbol] 13 | 0 | 0 | 0 | 0 | 0 |
| [symbol] -1 | | | | | | | | [symbol] | 0 | [symbol] | 87,121 | 0 | 0 | 0 | 0 | 0 |
| [symbol] -2 | | | | | | | | 778 | 282 | 11,3575 | 44,171 | 0 | 0 | 0 | 0 | 0 |
| [symbol] -3 | | | | | | | | [symbol] | [symbol] | 89,117 | [symbol] | 0 | 0 | 0 | 0 | 0 |
| [symbol] -4 | | | | | | | | 64 | 3575 | [symbol] | 11,740 | 0 | 0 | 0 | 0 | 0 |
| [symbol] -5 | | | | | | | | 647 | 217 | [symbol] | 34,175 | 0 | 0 | 0 | 0 | 0 |
| [symbol] -6 | | | | | | | | 1734 | 0 | 48,374 | 16,000 | 0 | 0 | 0 | 0 | 0 |
| A [symbol] -0 | | | | | | | wt | 147 | 1853 | 35,454 | 11,740 | 0 | 0 | 0 | 0 | 0 |
| EXVX-8 | | | | | | | [symbol] | 261 | [symbol] | 74,732 | 34,175 | 0 | 0 | 0 | 0 | 0 |
| HGT-118-7 | | | | | | | wt | 438 | 0 | 48,374 | 16,076 | 0 | 0 | 0 | 0 | 0 |
| HGT-118-8 | | | | | | | wt | 368 | 674 | [symbol] | [symbol] | 0 | 0 | 0 | 0 | 0 |
| HT25-1 | | | | | | | [symbol] | 273 | 3281 | 23,164 | 16,610 | 0 | 0 | 0 | 0 | 0 |
| XT25-1 | | | | | | | [symbol] | 78 | 3147 | [symbol] | 6481 | 0 | 0 | 0 | 0 | 0 |
| HT25-6 | | | | | | | [symbol] | 181 | 457 | 63,732 | 14,319 | 0 | 0 | 0 | 0 | 0 |
| SF [symbol] -7 | | | | | | | wt | 107 | 446 | 33,067 | 13,17 | 0 | 0 | 0 | 0 | 0 |
| SF538-8 | | | | | | | wt | 223 | 0 | 29,182 | 14,319 | 0 | 0 | 0 | 0 | 0 |
| SF266-7 | | | | | | | [symbol] | 881 | 1301 | 72,107 | 6447 | 0 | 0 | 0 | 0 | 0 |
| SF-366-6 | | | | | | | [symbol] | 1384 | 627 | 10,500 | 8738 | 0 | 0 | 0 | 0 | 0 |
| OVCAR-4-7 | | | | | | | wt | 0 | 320 | 12,000 | 13,175 | 0 | 0 | 0 | 0 | 0 |
| OVCAR-5-8 | | | | | | | [symbol] | 154 | 0 | 17,814 | 0041 | 0 | 0 | 0 | 0 | 0 |
| OVCAR-6-7 | | | | | | | [symbol] | 545 | 740 | 24,120 | 14,422 | 0 | 0 | 0 | 0 | 0 |
| OVCAR-6-8 | | | | | | | [symbol] | 422 | 3013 | 12,600 | 21,781 | 0 | 0 | 0 | 0 | 0 |
| MCF-7-8 | | | | | | | [symbol] | 0 | 2343 | 23,063 | 10,770 | 0 | 0 | 0 | 0 | 0 |
| [symbol] | | | | | | | [symbol] | 737 | 1041 | 24,571 | 10,657 | 0 | 0 | 0 | 0 | 0 |
| [symbol] | | | | | | | [symbol] | 224 | 7324 | 43,102 | 11,163 | 0 | 0 | 0 | 0 | 0 |
| SW489-7 | | | | | | | [symbol] | 102 | 2330 | [symbol] | 17,722 | 0 | 0 | 0 | 0 | 0 |
| SW489-8 | | | | | | | [symbol] | 372 | 2172 | 11,400 | 6104 | 0 | 0 | 0 | 0 | 0 |
| H1238-8 | | | | | | | [symbol] | 0 | 1613 | 28,317 | 12,001 | 0 | 0 | 0 | 0 | 0 |
| C33A-7 | | | | | | | [symbol] | 70 | 2184 | 72,532 | [symbol] | 0 | 0 | 0 | 0 | 0 |
| C33A-8 | | | | | | | [symbol] | 67 | 0 | 15,0,067 | 10,781 | 0 | 0 | 0 | 0 | 0 |
| U203-7 | | | | | | | wt | 538 | 4034 | 38,274 | 8774 | 0 | 0 | 0 | 0 | 0 |
| U308-8 | | | | | | | wt | 502 | 4457 | 8255 | 13,570 | 0 | 0 | 0 | 0 | 0 |
| [symbol] -7 | | | | | | | wt | 732 | 47 | [symbol] | 14,777 | 0 | 0 | 0 | 0 | 0 |
| [symbol] -8 | | | | | | | | 6548 | 0 | [symbol] | 10,004 | 0 | 0 | 0 | 0 | 0 |
| WI38-8 | | | | | | | | 110 | 0 | 8735 | 7518 | 0 | 0 | 0 | 0 | 0 |
| [symbol] | | | | | | 64 | | 341 | 1575 | [symbol] | 7421 | 15,778 | 21,839 | 3228 | 37 [symbol] | 7006 |
| CF2,1572, [symbol] | | | | | | | | 1340 | 405 | 87 [symbol] | 2767 | 887 | 2381 | 1237 | [symbol] | 2084 |
| [symbol] | | | | | | | | 457 | 516 | 1 [symbol] | [symbol] | 4834 | 12,083 | 23,343 | 21,752 | 2324 |
| HT388 | | | | | | | | 3175 | 0 | [symbol] | 24,204 | 4834 | 5347 | 715 | [symbol] | 7005 |
| HT345 | | | | | | | | 0 | 380 | [symbol] 198 | [symbol] 34 | 7363 | 15,311 | 725 | [symbol] | 5768 |
| HT675 | | | | | | | | 40 | 303 | 2532 | 2582 | 704 | 7809 | 2635 | [symbol] | 7837 |
| HT628 | | | | | | | | 01 | 420 | 8381 | 4229 | 183 | 2123 | 2338 | [symbol] | 2764 |
| [symbol] -3 | | | | | | 173 | | 325 | 0 | 14,405 | 12,819 | 1029 | 153 | 723 | 45,163 | [symbol] |
| [symbol] -6 | | | | | | 175 | | 1003 | 0 | [symbol] | 5200 | 859 | 1780 | 121 | [symbol] | 4314 |
| [symbol] -8 | | | | | | 177 | | 591 | 0 | 83 [symbol] | 5200 | 1536 | 1118 | 115 | [symbol] | [symbol] |
| [symbol] | | | | | | | | 138 | 0 | 14,405 | 12,819 | 1542 | 3275 | 1342 | [symbol] | 1011 |
| [symbol] 10 | | | | | | 237 | | 515 | 0 | [symbol] | [symbol] | 1666 | 1,8010 | 2231 | [symbol] | 14,731 |
| HT10 | | | | | | | | 384 | 37 | 2723 | 5219 | 263 | 3078 | 1114 | [symbol] | 8700 |
| [symbol] | | | | | | | | 405 | 0 | 2,00,879 | 11,083 | 1553 | 367 | 74 | 44,374 | 8400 |
| [symbol] | | | | | | | | 1201 | 0 | 29,774 | 11,028 | 2188 | 10, [symbol] | 1479 | 24,578 | 4500 |
| [symbol] | | | | | | | | 1883 | 354 | 75,223 | 17,508 | 3422 | 1$ | 1384 | 27,087 | 4355 |
| [symbol] | | | | | | | wt | 420 | 3054 | [symbol] | [symbol] | 3824 | 3415 | 2424 | 32,124 | 7705 |
| [symbol] | | | | | | | wt | 857 | 0 | 29,779 | [symbol] | 0 | 0 | 0 | 0 | 0 |
| HCT-176-3 | | | | | | | wt | 350 | 377 | 30,555 | 13,023 | 0 | 0 | 0 | 0 | 0 |
| HCT-116-4 | | | | | | | wt | 0 | 271 | [symbol] | [symbol] | 0 | 0 | 0 | 0 | 0 |
| HCT-116-5 | | | | | | | wt | 0 | 248 | 2330 | 7224 | 0 | 0 | 0 | 0 | 0 |
| HCT-116-6 | | | | | | | [symbol] | [symbol] | 4538 | 3330 | 7224 | 0 | 0 | 0 | 0 | 0 |
| AS [symbol] | | | | | | | [symbol] | 238 | 3771 | 3915 | 0 | 0 | 0 | 0 | 0 | 0 |
| NT29-3 | | | | | | | [symbol] | 27 | 0 | 62,803 | 8937 | 0 | 0 | 0 | 0 | 0 |
| NT29-3 | | | | | | | [symbol] | 448 | 0 | [symbol] | 8240 | 0 | 0 | 0 | 0 | 0 |
| NT29-4 | | | | | | | [symbol] | 329 | 1308 | 23,073 | 18,179 | 0 | 0 | 0 | 0 | 0 |
| NT29-5 | | | | | | | wt | 661 | 457 | 63,479 | [symbol] | 0 | 0 | 0 | 0 | 0 |
| NT29-6 | | | | | | | wt | 329 | 3832 | 64,009 | 44,924 | 0 | 0 | 0 | 0 | 0 |
| OVCAR-4-3 | | | | | | | wt | 0 | 522 | [symbol] | 8741 | 0 | 0 | 0 | 0 | 0 |
| OVCAR-4-4 | | | | | | | wt | 391 | 0 | 53,852 | 13,001 | 0 | 0 | 0 | 0 | 0 |
| OVCAR-4-5 | | | | | | | wt | 291 | 0 | 83 [symbol] | [symbol] | 0 | 0 | 0 | 0 | 0 |
| OVCAR-4-6 | | | | | | | wt | 404 | 617 | 27,709 | 14 [symbol] | 0 | 0 | 0 | 0 | 0 |
| SF [symbol] -3 | | | | | | | wt | 259 | 2513 | 53,162 | 9722 | 0 | 0 | 0 | 0 | 0 |
| SF [symbol] -4 | | | | | | | wt | 0 | 3084 | 44,663 | [symbol] | 0 | 0 | 0 | 0 | 0 |
| SF [symbol] -5 | | | | | | | [symbol] | 648 | 444 | 8763 | 7202 | 0 | 0 | 0 | 0 | 0 |
| SF [symbol] -6 | | | | | | | wt | 0 | 617 | 27,709 | 14 [symbol] | 0 | 0 | 0 | 0 | 0 |
| OVCAR-4-3 | | | | | | | [symbol] | 0 | 892 | 15,534 | 7377 | 0 | 0 | 0 | 0 | 0 |
| OVCAR-4-4 | | | | | | | [symbol] | 512 | 2112 | 90,711 | 12,219 | 0 | 0 | 0 | 0 | 0 |
| OVCAR-4-5 | | | | | | | [symbol] | [symbol] | [symbol] | 82,509 | [symbol] | 0 | 0 | 0 | 0 | 0 |
| OVCAR-5-6 | | | | | | | [symbol] | 215 | 0 | 82 [symbol] | [symbol] | 0 | 0 | 0 | 0 | 0 |
| WCF-7-0 | | | | | | | [symbol] | 711 | 254 | 21,094 | 11,567 | 0 | 0 | 0 | 0 | 0 |
| [symbol] -6 | | | | | | | [symbol] | 0 | 226 | 50,552 | 8767 | 0 | 0 | 0 | 0 | 0 |
| H1230-1 | | | | | | | [symbol] | 313 | 3044 | [symbol] | 17,055 | 0 | 0 | 0 | 0 | 0 |
| SW480-3 | | | | | | | [symbol] | 824 | 0 | [symbol] | 25,234 | 0 | 0 | 0 | 0 | 0 |
| SW480-4 | | | | | | | [symbol] | 84 | 240 | [symbol] | [symbol] | 0 | 0 | 0 | 0 | 0 |
| SW480-5 | | | | | | | [symbol] | 822 | 0 | 40 [symbol] | 21,184 | 0 | 0 | 0 | 0 | 0 |
| SW480-6 | | | | | | | [symbol] | 0 | 0 | 42,319 | 25,174 | 0 | 0 | 0 | 0 | 0 |
| C33A-3 | | | | | | | [symbol] | 858 | 211 | 77,746 | 11,233 | 0 | 0 | 0 | 0 | 0 |
| C33A-4 | | | | | | | [symbol] | 254 | 875 | [symbol] | [symbol] | 0 | 0 | 0 | 0 | 0 |
| C33A-5 | | | | | | | [symbol] | 428 | 0 | 9205 | 7527 | 0 | 0 | 0 | 0 | 0 |
| C33A-6 | | | | | | | [symbol] | 144 | 85 | 2,17,524 | 30,812 | 0 | 0 | 0 | 0 | 0 |
| [symbol] | | | | | | | [symbol] | 353 | 2141 | 1,45,783 | 12,529 | 0 | 0 | 0 | 0 | 0 |
| UQO3-3 | | | | | | | [symbol] | 367 | 1378 | 11,555 | 17,142 | 0 | 0 | 0 | 0 | 0 |
| UQO3-4 | | | | | | | [symbol] | 817 | 128 | 11,177 | 12,775 | 0 | 0 | 0 | 0 | 0 |
| UQO3-5 | | | | | | | [symbol] | 825 | 404 | [symbol] | [symbol] | 0 | 0 | 0 | 0 | 0 |
| UQO3-6 | | | | | | | [symbol] | 434 | 4041 | 57,189 | 18,944 | 0 | 0 | 0 | 0 | 0 |
| [symbol] | | | | | | | [symbol] | 25 | 4744 | [symbol] | [symbol] | 0 | 0 | 0 | 0 | 0 |
| [symbol] -3 | | | | | | | wt | 232 | 1334 | 57,073 | 17,844 | 0 | 0 | 0 | 0 | 0 |
| [symbol] -6 | | | | | | | wt | 1334 | 140 | [symbol] | 27,837 | 0 | 0 | 0 | 0 | 0 |
| SF-366-3 | | | | | | | wt | 1761 | 6402 | 18,667 | 21,304 | 0 | 0 | 0 | 0 | 0 |
| SF-366-4 | | | | | | | [symbol] | 1145 | 1850 | 30,421 | 12,423 | 0 | 0 | 0 | 0 | 0 |
| SF-366-5 | | | | | | | [symbol] | 821 | 1858 | 40,854 | 20,121 | 0 | 0 | 0 | 0 | 0 |
| SF-366-6 | | | | | | | [symbol] | 574 | 1530 | 1,27,423 | 12,423 | 0 | 0 | 0 | 0 | 0 |
| [symbol] -13 | | | | | | | [symbol] | 1771 | 1629 | 2,35,833 | [symbol] | 0 | 0 | 0 | 0 | 0 |
| [symbol] -29 | | | | | | | | 1817 | [symbol] | [symbol] | [symbol] | 0 | 0 | 0 | 0 | 0 |
| [symbol] -21 | | | | | | | | 1349 | 87 | 34,349 | 63,737 | 0 | 0 | 0 | 0 | 0 |
| [symbol] -22 | | | | | | | | 1779 | 2514 | 1,20,280 | 25,282 | 0 | 0 | 0 | 0 | 0 |
| OVCAR-4-6 | | | | | | | [symbol] | 1023 | 0 | 41,280 | 27,874 | 0 | 0 | 0 | 0 | 0 |
| [symbol] -10 | | | | | | | | 1347 | 15 | 79,144 | 73,812 | 0 | 0 | 0 | 0 | 0 |
| [symbol] -11 | | | | | | | | 613 | 0 | 14 [symbol] | 87,514 | 0 | 0 | 0 | 0 | 0 |
| [symbol] -12 | | | | | | | | 1081 | 0 | 1 [symbol] | 5 [symbol] | 0 | 0 | 0 | 0 | 0 |
| [symbol] -13 | | | | | | | | 2111 | 0 | 24 [symbol] | [symbol] | 0 | 0 | 0 | 0 | 0 |
| [symbol] -14 | | | | | | | | 2004 | 0 | 58,283 | [symbol] | 0 | 0 | 0 | 0 | 0 |
| [symbol] -15 | | | | | | | | 739 | 0 | 1,04,457 | 14,747 | 0 | 0 | 0 | 0 | 0 |
| [symbol] -16 | | | | | | | | 818 | 0 | 96,744 | 13,838 | 0 | 0 | 0 | 0 | 0 |
| [symbol] -17 | | | | | | | | 675 | 0 | 40,277 | 10,300 | 0 | 0 | 0 | 0 | 0 |
| [symbol] -18 | | | | | | | | 480 | 557 | 1,44,750 | 18,832 | 0 | 0 | 0 | 0 | 0 |
| [symbol] -19 | | | | | | | | 844 | 4 | 3,00,623 | [symbol] | 0 | 0 | 0 | 0 | 0 |
| adrenal gland | | 1 | | | | | | [symbol] | 1342 | 187782 | 8831 | [symbol] | [symbol] | [symbol] | [symbol] | 4309 |
| [symbol] | | 2 | | | | | | [symbol] | 1034 | [symbol] | 14301 | [symbol] | [symbol] | [symbol] | [symbol] | 2871 |
| [symbol] | | 3 | | | | | | [symbol] | 345 | 117700 | 11387 | 21823 | [symbol] | [symbol] | 0 | [symbol] |
| [symbol] | | 4 | | | | | | 34733 | 0 | [symbol] | [symbol] | 723 | [symbol] | [symbol] | [symbol] | 344 |
| [symbol] | | 5 | | | | | | [symbol] | [symbol] | [symbol] | 1470 | 829 | [symbol] | [symbol] | [symbol] | 2311 |
| [symbol] | | 6 | | | | | | [symbol] | 2034 | [symbol] | 1439 | 7237 | [symbol] | [symbol] | [symbol] | 1244 |
| [symbol] | | 7 | | | | | | [symbol] | 134 | [symbol] | 10723 | 2768 | [symbol] | [symbol] | [symbol] | 274 |
| [symbol] | | 8 | | | | | | [symbol] | 232 | [symbol] | [symbol] | 7314 | [symbol] | [symbol] | [symbol] | 3472 |
| [symbol] | | 9 | | | | | | 103271 | 0 | [symbol] | 22934 | 3834 | [symbol] | [symbol] | [symbol] | [symbol] |
| [symbol] | | 10 | | | | | | [symbol] | [symbol] | [symbol] | 1973 | 8434 | [symbol] | [symbol] | [symbol] | [symbol] |
| [symbol] | | 11 | | | | | | [symbol] | [symbol] | [symbol] | [symbol] | 12334 | [symbol] | [symbol] | [symbol] | 4617 |
| [symbol] | | 12 | | | | | | [symbol] | 1446 | [symbol] | [symbol] | 17732 | 1143 | [symbol] | 870 | [symbol] |
| [symbol] | | 13 | | | | | | [symbol] | 3183 | [symbol] | 11342 | 18732 | 3479 | [symbol] | 270 | 2730 |
| [symbol] | | 14 | | | | | | [symbol] | 0 | [symbol] | [symbol] | 18787 | 1000 | [symbol] | 0 | 233 |
| [symbol] | | 15 | | | | | | [symbol] | 1470 | [symbol] | 8127 | 18343 | 4170 | [symbol] | 1544 | 3495 |
| [symbol] | | 16 | | | | | | [symbol] | 171 | [symbol] | 8744 | 1837 | [symbol] | [symbol] | 2077 | 3017 |
| [symbol] | | 17 | | | | | | [symbol] | 0 | [symbol] | [symbol] | 4454 | 2345 | [symbol] | 1102 | 3134 |
| [symbol] | | 18 | | | | | | [symbol] | 1342 | [symbol] | 3431 | 14330 | 2831 | [symbol] | 532 | 1432 |
| [symbol] | | 19 | | | | | | [symbol] | [symbol] | [symbol] | [symbol] | 3332 | [symbol] | [symbol] | [symbol] | 1434 |
| [symbol] | | 20 | | | | | | [symbol] | 1315 | 53517 | 21224 | 5273 | [symbol] | [symbol] | 787 | 714 |
| [symbol] | | 21 | | | | | | [symbol] | [symbol] | [symbol] | 4474 | [symbol] | 1234 | [symbol] | 124 | 413 |
| [symbol] | | 22 | | | | | | [symbol] | 0 | [symbol] | [symbol] | 30444 | 4242 | 17845 | 0 | 822 |
| [symbol] | | 23 | | | | | | 37827 | 577 | [symbol] | [symbol] | [symbol] | [symbol] | [symbol] | 0 | [symbol] |
| [symbol] | | 24 | | | | | | [symbol] | [symbol] | [symbol] | [symbol] | [symbol] | [symbol] | 71833 | [symbol] | 331 |
| [symbol] | | 25 | | | | | | [symbol] | [symbol] | [symbol] | [symbol] | 12224 | 454 | [symbol] | [symbol] | 1333 |
| [symbol] | | 27 | | | | | | 85252 | 83 | [symbol] | [symbol] | [symbol] | 8770 | [symbol] | 1733 | 317 |
| [symbol] | | 28 | | | | 28 | | [symbol] | 0 | 3134 | [symbol] | 2437 | [symbol] | 0 | 724 | 0 |
| [symbol] | | 29 | | | | | | [symbol] | 642 | [symbol] | 1932 | 4819 | 824 | 37702 | 0 | 755 |
| [symbol] | | 30 | | | | 30 | | [symbol] | 0 | 3012 | [symbol] | 4347 | 344 | [symbol] | 0 | 0 |
| [symbol] | | 31 | | | | | | [symbol] | [symbol] | 144782 | 1222 | 30444 | 3321 | [symbol] | 402 | [symbol] |
| [symbol] | | 32 | | | | | | [symbol] | [symbol] | 21473 | 3421 | 0 | 3472 | 37045 | 387 | 0 |
| [symbol] | | 33 | | | | | | [symbol] | 354 | [symbol] | 1371 | 4733 | 1003 | [symbol] | [symbol] | 3431 |
| [symbol] | | 34 | | | | | | 81717 | 373 | [symbol] | 2920 | 113 | 540 | [symbol] | 342 | 0 |
| [symbol] | | 35 | | | | | | [symbol] | 0 | 0 | [symbol] | 13335 | 0 | [symbol] | 0 | [symbol] |
| [symbol] | | 36 | | | | | | [symbol] | 0 | 0 | [symbol] | [symbol] | 2110 | [symbol] | [symbol] | 0 |
| [symbol] | | 37 | | | | | | 30349 | 418 | [symbol] | 4134 | [symbol] | [symbol] | [symbol] | 812 | 0 |
| [symbol] | | 38 | | | | | | [symbol] | [symbol] | 0 | [symbol] | 9199 | 1004 | [symbol] | 847 | 256 |
| [symbol] | | 39 | | | | | | 47672 | 0 | 2918 | [symbol] | 1134 | 0 | [symbol] | 482 | 0 |
| [symbol] | | 40 | | | | | | 70722 | 0 | 0 | 3341 | [symbol] | [symbol] | [symbol] | 220 | 0 |
| [symbol] | | 41 | | | | | | 42181 | 143 | 0 | [symbol] | 4470 | [symbol] | [symbol] | 10 | 3041 |
| [symbol] | | 42 | | | | | | [symbol] | [symbol] | 0 | 6154 | 4109 | 205 | 1081 | [symbol] | 304 |
| [symbol] | | 43 | | | | | | 25172 | 40 | 827 | 1379 | 17131 | 0 | 1708 | 0 | 0 |
| [symbol] | | 44 | | | | | | [symbol] | 120 | 234 | [symbol] | 13874 | 402 | 3483 | 334 | 0 |
| [symbol] | | | | | [symbol] | | | [symbol] | 0 | 0 | [symbol] | 0 | 57 | 0 | 244 | 0 |
| [symbol] | | | | | [symbol] | | | 8249 | 77 | 0 | 244 | 333 | 347 | 337 | [symbol] | 0 |
| [symbol] | | | | | [symbol] | | | 16329 | 0 | 0 | 249 | 183 | 0 | [symbol] | 0 | 0 |
| [symbol] | | | | | 394 | 394 | | 82144 | [symbol] | 0 | 23733 | 6784 | 7222 | 8308 | 1400 | 12 |
| [symbol] | | | | | 354 | 354 | | [symbol] | [symbol] | 0 | [symbol] | 4027 | 1113 | 422 | 474 | 0 |
| [symbol] | | | | | 344 | | | 27729 | 0 | 0 | 238 | 839 | 90 | 0 | 0 | 71 |
| [symbol] | | | | | 342 | | | 31432 | 230 | 0 | 0 | 54 | 79 | 0 | 0 | 0 |
| [symbol] | | | | | 334 | 334 | | 71833 | 0 | 0 | [symbol] | 2500 | 61 | [symbol] | 113 | 0 |
| [symbol] | | | | | 333 | | | 30788 | 277 | 335 | [symbol] | 52857 | 253 | 347 | 0 | 0 |
| [symbol] | | | | | 330 | | | [symbol] | 237 | 0 | 700 | 41 | 0 | 223 | 54 | 0 |
| [symbol] | | | | | 329 | | | [symbol] | 0 | 8377 | 20506 | 12409 | 83 | 2748 | 1212 | 0 |
| [symbol] | | | | | 327 | | | [symbol] | 0 | [symbol] | 0 | 10838 | 364 | 302 | 1110 | 0 |
| [symbol] | | | | | [symbol] | | | [symbol] | 0 | [symbol] | 42378 | 241101 | 721 | 780 | 524 | [symbol] |
| [symbol] | | | | | 321 | | | 41371 | 416 | 0 | 0 | 0 | 43 | [symbol] | 338 | 0 |
| [symbol] | | | | | 320 | | | [symbol] | 0 | 14728 | 4706 | [symbol] | 225 | [symbol] | 232 | 0 |
| [symbol] | | | | | 319 | | | [symbol] | 644 | 2041 | [symbol] | 300533 | [symbol] | 4242 | 1207 | [symbol] |
| [symbol] | | | | | 318 | | | [symbol] | 574 | 0 | [symbol] | [symbol] | [symbol] | 2740 | 403 | 440 |
| [symbol] | | | | | 314 | | | [symbol] | 0 | [symbol] | [symbol] | [symbol] | 1208 | [symbol] | 0 | [symbol] |
| [symbol] | | | | | 311 | | | [symbol] | 0 | 0 | [symbol] | 30382 | 1948 | 547 | 0 | 0 |
| [symbol] | | | | | 309 | | | [symbol] | [symbol] | 0 | [symbol] | 25475 | [symbol] | [symbol] | 134 | 0 |
| [symbol] | | | | | 307 | | | [symbol] | 118 | 0 | [symbol] | 18211 | [symbol] | [symbol] | [symbol] | 157 |
| [symbol] | | | | | [symbol] | | | 44817 | 0 | 0 | 831 | [symbol] | 1321 | 520 | 124 | [symbol] |
| [symbol] | | | | | [symbol] | | | [symbol] | 1164 | 0 | 8442 | [symbol] | 7874 | [symbol] | 223 | 0 |
| [symbol] | | | | | [symbol] | | | 72421 | [symbol] | 3702 | 8772 | [symbol] | [symbol] | 4725 | [symbol] | [symbol] |
| [symbol] | | | | | 299 | | | [symbol] | 444 | 0 | 35622 | [symbol] | 8342 | [symbol] | 1714 | 0 |
| [symbol] | | | | | 297 | | | [symbol] | 0 | 1047 | 34177 | [symbol] | [symbol] | 3674 | [symbol] | 51 |
| [symbol] | | | | | 298 | | | [symbol] | 0 | 138 | 2172 | [symbol] | [symbol] | 504 | 0 | 0 |
| [symbol] | | | | | 294 | | | [symbol] | [symbol] | 0 | 23722 | 38430 | 3182 | 1843 | 1180 | 134 |
| [symbol] | | | | | 292 | | | [symbol] | 36 | 0 | [symbol] | [symbol] | 8303 | [symbol] | 0 | 133 |
| [symbol] | | | | | 290 | | | [symbol] | 0 | [symbol] | [symbol] | 83231 | [symbol] | 2424 | 425 | [symbol] |
| [symbol] | | | | | 279 | | | [symbol] | 27 | 0 | 0 | 27722 | [symbol] | 0 | 0 | 0 |
| [symbol] | | | | | 277 | | | [symbol] | 27 | 0 | 2305 | [symbol] | [symbol] | 0 | 0 | 365 |
| [symbol] | | | | | 273 | 273 | | [symbol] | 0 | 0 | 0 | 1157 | 0 | 432 | 0 | 0 |
| [symbol] | | | | | [symbol] | | | [symbol] | 304 | 0 | 0 | 20354 | 0 | 141 | 804 | 0 |
| [symbol] | | | | | [symbol] | | | [symbol] | 0 | 1370 | 0 | 3833 | 0 | 304 | 0 | 405 |
| [symbol] | | | | | [symbol] | [symbol] | | 73347 | 1164 | 0 | 1100 | [symbol] | [symbol] | 14 | 218 | 144 |
| [symbol] | | | | | 235 | 235 | | [symbol] | [symbol] | 828 | 100 | 2281 | 857 | 0 | 0 | 0 |
| [symbol] | | | | | 234 | | | 78287 | 0 | 1108 | 2007 | 13754 | 0 | 0 | 0 | 0 |
| [symbol] | | | | | 233 | 233 | | [symbol] | 374 | 0 | 1052 | 1379 | 784 | 0 | 501 | 0 |
| [symbol] | | | | | 231 | 231 | | 62712 | 0 | 307 | 0 | 1571 | 1227 | 0 | 73 | [symbol] |
| [symbol] | | | | | 229 | 229 | | [symbol] | 0 | 581 | 2192 | 2318 | [symbol] | 0 | 574 | 0 |
| [symbol] | | | | | 227 | 227 | | [symbol] | 0 | 0 | 783 | 1830 | 842 | 0 | 0 | 0 |
| [symbol] | | | | | 222 | | | 33431 | 305 | 0 | [symbol] | [symbol] | 87 | 0 | 54 | 0 |
| [symbol] | | | | | 215 | | | [symbol] | 0 | 228 | 744 | [symbol] | 0 | 0 | 130 | 0 |
| [symbol] | | | | | 214 | | | [symbol] | 0 | [symbol] | [symbol] | 52831 | 2007 | 323 | 679 | 0 |
| [symbol] | | | | | 213 | | | [symbol] | 483 | 0 | 2249 | 743 | 43 | 334 | 131 | 0 |
| [symbol] | | | | | 212 | | | [symbol] | 0 | 0 | 0 | 11411 | [symbol] | 0 | 347 | 0 |
| [symbol] | | | | | 211 | 211 | | 34737 | 290 | 34 | [symbol] | 0 | 0 | 0 | 0 | 43 |
| [symbol] | | | | | 210 | | | 70127 | 0 | 0 | 23 | [symbol] | [symbol] | 0 | 443 | 0 |
| [symbol] | | | | | 209 | | | [symbol] | 155 | 67 | [symbol] | 2433 | [symbol] | 1106 | 0 | 0 |
| [symbol] | | | | | 208 | | | [symbol] | 0 | 0 | [symbol] | 7304 | [symbol] | 222 | [symbol] | 120 |
| [symbol] | | | | | [symbol] | | | [symbol] | 0 | 0 | 962 | 34 | 0 | 0 | 323 | 204 |
| [symbol] | | | | | 201 | | | 33307 | 0 | [symbol] | 0 | [symbol] | 0 | 545 | 0 | 0 |
| [symbol] | | | | | 199 | | | [symbol] | 0 | 0 | 179 | [symbol] | 0 | 0 | 0 | 0 |
| [symbol] | | | | | 197 | | | [symbol] | 0 | 8020 | [symbol] | [symbol] | 0 | [symbol] | 53 | 0 |
| [symbol] | | | | | [symbol] | | | 21318 | [symbol] | [symbol] | 1721 | 379 | [symbol] | 34 | 186 | 0 |
| [symbol] | | | | | [symbol] | | | 23113 | [symbol] | 0 | 14220 | [symbol] | 8113 | 792 | 505 | 0 |
| [symbol] | | | | | [symbol] | | | [symbol] | 0 | 0 | 0 | [symbol] | 0 | [symbol] | 0 | 0 |
| [symbol] | | | | | [symbol] | | | [symbol] | 0 | 0 | 231 | [symbol] | 3067 | 218 | 83 | 0 |
| [symbol] | | | | | 19 | | | [symbol] | [symbol] | 0 | 2734 | [symbol] | 7184 | 840 | 454 | 0 |
| [symbol] | | | | | 17 | | | [symbol] | 0 | 0 | 0 | 7921 | [symbol] | 449 | 134 | 347 |
| [symbol] | | | | | 14 | | | 37903 | 0 | [symbol] | [symbol] | [symbol] | 436 | 0 | 0 | 0 |
| [symbol] | | | | | 13 | | | 12835 | 0 | 0 | 778 | 7737 | 223 | 82 | 0 | 0 |
| [symbol] | | | | | 11 | | | 21541 | [symbol] | 0 | 342 | 7518 | [symbol] | 149 | 0 | 0 |
| [symbol] | | | | | 10 | | | 31744 | 244 | 37 | 341 | [symbol] | 154 | 141 | [symbol] | [symbol] |
| [symbol] | | | | 79 | | | | [symbol] | 134 | 0 | 144 | 0 | 600 | 507 | 419 | 0 |
| [symbol] | | | | 81 | | | | 17302 | 0 | 0 | 78 | 462 | [symbol] | [symbol] | 0 | 0 |
| [symbol] | | | | 83 | | | | 63322 | 0 | 0 | 0 | 0 | [symbol] | 0 | 714 | 0 |
| [symbol] | | | | 84 | | | | 28774 | [symbol] | 0 | 1042 | 0 | 827 | [symbol] | 0 | 0 |
| [symbol] | | | | 89 | | | | [symbol] | 0 | 0 | 3037 | 804 | 231 | [symbol] | 185 | 0 |
| [symbol] | | | | 90 | | | | [symbol] | 0 | 0 | [symbol] | 1347 | 1187 | 385 | 1438 | 0 |
| [symbol] | | | | 92 | | | | 52833 | 0 | 0 | 0 | 1254 | [symbol] | 0 | 185 | 0 |
| [symbol] | | | | 94 | | | | 43442 | 0 | 0 | 0 | 836 | [symbol] | [symbol] | 0 | 0 |
| [symbol] | | | | 99 | | | | 34824 | [symbol] | 0 | 0 | [symbol] | [symbol] | 0 | 313 | 0 |
| [symbol] | | | | 100 | | | | 15800 | 147 | 0 | 0 | [symbol] | 3313 | [symbol] | [symbol] | 189 |
| [symbol] | | | | 144 | | | | 22032 | 0 | 1157 | 0 | 718 | 1753 | 401401 | 87 | 282 |
| [symbol] | | | | 150 | | | | [symbol] | 72 | 0 | 0 | 539 | 701 | 00 | 183 | 357 |
| [symbol] | | | | 152 | | | | 17275 | 0 | 211 | 241 | 384 | 3481 | 545 | 0 | 57 |
| [symbol] | | | | 154 | | | | 14225 | 0 | 795 | 0 | 11 | [symbol] | 114114 | [symbol] | 178 |
| [symbol] | | | | 154 | | | | 8334 | 0 | [symbol] | 232 | 154 | [symbol] | 00 | 0 | 0 |
| [symbol] | | | | 154 | | | | [symbol] | 114 | 0 | 107 | 110 | [symbol] | 2424 | 143 | 141 |
| [symbol] | | | | 160 | | | | [symbol] | 350 | 0 | 234 | 409 | [symbol] | 00 | 20 | 0 |
| [symbol] | | | | 162 | | | | 14306 | 123 | 0 | [symbol] | 317 | [symbol] | 201 | 0 | 778 |
| [symbol] | | | | 164 | | | | 13034 | 0 | 231 | 370 | 422 | [symbol] | 0 | 670 | 0 |
| [symbol] | | | | [symbol] | | | | [symbol] 41 | 1 | 9 | 177 | [symbol] 71 | 4172 | 0 | 0 | 0 |
| [symbol] | | | | 1 [symbol] | | | | [symbol] 23 | 1415 | 0 | [symbol] | [symbol] | 5374 | 100 | 0 | [symbol] |
| [symbol] | | | | 1 [symbol] | | | | [symbol] | [symbol] | [symbol] | 438 | 731 | 1318 | 1100 | 0 | 124 |
| [symbol] | | | | 1 [symbol] | | | | [symbol] 45 | 47 | [symbol] 22 | [symbol] | 0 | [symbol] | 0 | 0 | 0 |
| [symbol] | | | | 1 [symbol] | | | | 11 [symbol] | [symbol] | 210 | 1871 | 247 | 334 | 32 | 187 | 176 |
| [symbol] | | | | 171 | | | | [symbol] | 0 | [symbol] | 0 | 0 | 0 | [symbol] 1 | 120 | 126 |
| [symbol] | | | | 187 | | | | [symbol] | 0 | 0 | [symbol] | 3000 | 1000 | [symbol] | 0 | 0 |
| [symbol] | | | | 1 [symbol] | | | | [symbol] | 0 | 0 | 1154 | 3112 | 1 [symbol] 2 | 141 | 06 | 30 |
| [symbol] | | | | 200 | | | | [symbol] | 130 | 0 | [symbol] | 523 | 174 | 750 | 371 | 130 |
| [symbol] | | | | 202 | | | | [symbol] | 7143 | 1474 | [symbol] | [symbol] | [symbol] | 0 | [symbol] | 0 |
| [symbol] | | | | 206 | | | | [symbol] | 0 | [symbol] | 714 | [symbol] | 211 | 174 | 0 | 433 |
| [symbol] | | | | 274 | | | | [symbol] | 0 | 0 | 1234 | 572 | 143 | 0 | [symbol] | 0 |
| [symbol] | | | | 220 | | | | [symbol] | 0 | 0 | 14 | 0 | 02 | 0 | 330 | 0 |
| [symbol] | | | | 220 | | | | [symbol] | 0 | 0 | 3 [symbol] | 140 [symbol] | 411 | 0 | 411 | 0 |
| [symbol] | | | | 221 | | | | [symbol] | 0 | [symbol] 41 | 0 | 831 | 47 | 0 | [symbol] | 0 |
| [symbol] | | | | 270 | | | | [symbol] | 0 | [symbol] | 0 | 3727 | 0 | [symbol] | 174 | 134 |
| [symbol] | | | | 223 | | | | [symbol] | 0 | 0 | 0 | 0 | [symbol] | [symbol] | 0 | 0 |
| [symbol] | | | | 2 [symbol] | | | | [symbol] | 04 | 123 | [symbol] | [symbol] | 447 | 1 [symbol] 7 | 2 [symbol] 1 | 0 |
| [symbol] | | | | 221 | | | | [symbol] | 00 | 0 | 223 | 0 | 236 | 0 | 0 | 0 |
| [symbol] | | | | 2 [symbol] | | | | [symbol] | 43 | 0 | 134 | 0 | 23 | 0 | [symbol] | 0 |
| [symbol] | | | | 241 | | | | [symbol] | 0 | [symbol] | [symbol] | 724 | 140 | [symbol] | 0 | 0 |
| [symbol] | | | | 242 | | | | [symbol] | 0 | 0 | 142 | 22 | [symbol] | [symbol] | 0 | [symbol] |
| [symbol] | | | | 243 | | | | [symbol] | 367 | 13343 | 1834 | [symbol] | [symbol] | 371 | 0 | 0 |
| [symbol] | | | | 244 | | | | [symbol] | 0 | 200 | 0 | 0 | 4241 | 2403 | 0 | 0 |
| [symbol] | | | | 246 | | | | [symbol] | 100 | 0 | [symbol] | 307 | 360 | 1115 | 1154 | 1434 |
| [symbol] | | | | 248 | | | | [symbol] | 0 | 901 | 300 | [symbol] | [symbol] | 134 | 232 | [symbol] |
| [symbol] | | | | 250 | | | | [symbol] | 0 | 0 | [symbol] | 0 | 0 | 153 | [symbol] 12 | [symbol] |
| [symbol] | | | | 251 | | | | [symbol] | 0 | 0 | 0 | 354 | 723 | 0 | 007 | [symbol] |
| [symbol] | | | | 248 | | | | [symbol] | 1251 | 1251 | [symbol] | 152 | 1417 | 304 | [symbol] | 200 |
| [symbol] | | | | 240 | | | | [symbol] | 0 | 200 | 813 | 1004 | 2385 | 1074 | [symbol] | 0 |
| [symbol] | | | | 250 | | | | [symbol] | 0 | 1077 | 0 | 137 | 547 | 0 | 714 | 0 |
| [symbol] | | | | 251 | | | | [symbol] | 470 | 415 | 0 | 342 | 757 | 0 | 0 | 115 |
| [symbol] | | | | 262 | | | | [symbol] | 0 | 3782 | 1157 | 304 | [symbol] | 835 | 1034 | 271 |
| [symbol] | | | | 253 | | | | [symbol] | 0 | 3782 | 0 | 204 | 483 | 0 | 0 | 0 |
| [symbol] | | | | 254 | | | | [symbol] | 119 | [symbol] | [symbol] | 512 | 215 | 472 | 104 | 0 |
| [symbol] | | | | 256 | | | | [symbol] | [symbol] | [symbol] | 0 | 754 | [symbol] 133 | 0 | 270 | [symbol] |
| [symbol] | | | | 257 | | | | [symbol] | 120 | 573 | 740 | 373 | 1340 | 300 | 231 | [symbol] |
| [symbol] | | | | 258 | | | | [symbol] | 737 | [symbol] | 1917 | 0 | 235 | 377 | 73 | 0 |
| [symbol] | | | | 254 | | | | [symbol] | [symbol] 7 | [symbol] | 0 | 312 | [symbol] | 0 | 157 | 118 |
| [symbol] | | | | 300 | | | | [symbol] | [symbol] | [symbol] | 0 | 141 | 338 | 0 | [symbol] | 0 |
| [symbol] | | | | 301 | | | | [symbol] | 212 | 299 | 324 | [symbol] | 3 [symbol] | 0 | [symbol] | [symbol] |
| [symbol] | | | | 342 | | | | [symbol] | 0 | 0 | 617 | 1130 | [symbol] | 0 | 454 | 0 |
| [symbol] | | | | 343 | | | | [symbol] | [symbol] | 506 | [symbol] | 0 | [symbol] | [symbol] | [symbol] | [symbol] |
| [symbol] | | | | 343 | | | | [symbol] | [symbol] | 504 | [symbol] | 319 | 517 | [symbol] | 012 | 0 |
| [symbol] | | | | 3 [symbol] | | | | [symbol] | [symbol] | [symbol] | 0 | 170 | 34 | 279 | 0 | 0 |
| [symbol] | | | | 248 | | | | [symbol] | 0 | 1771 | [symbol] | 407 | 1109 | 2554 | 448 | 563 |
| [symbol] | | | | 247 | | | | [symbol] | 0 | 237 | 815 | 40 | 203 | 5 | 0 | 0 |
| [symbol] | | | | [symbol] | | | | [symbol] | 0 | 0 | [symbol] | 7537 | 0 | 418 | 3472 | 278 |
| [symbol] | | | | [symbol] | | | | [symbol] | 0 | 0 | 0 | 0 | 492 | 2743 | 424 | 424 |
| [symbol] | | | | 273 | | | | [symbol] | 727 | 2443 | 0 | 022 | [symbol] | [symbol] | [symbol] | 0 |
| Y | | | | [symbol] | | | | [symbol] | [symbol] | [symbol] | [symbol] | 8283 | 4347 | 1471 | 0 | [symbol] |
| K | | | | [symbol] | | | | [symbol] | 44 | 0 | 350 | [symbol] | 0 | 311 | 307 | 0 |
| H | | | | 273 | | | | [symbol] | 00 | 7043 | 0 | 0 | 31 | [symbol] | 42 | 0 |
| H | | | | 273 | | | | [symbol] | [symbol] | 2300 | [symbol] | 17200 | 27 [symbol] | 1120 | 103 | 0 |
| H | | | | 323 | | | | [symbol] | [symbol] | 0 | 205 | [symbol] | 0 | 1267 | 12 | 0 |
| [symbol] | | | | 324 | | | | [symbol] | 0 | 0 | [symbol] | 2011 | 0 | 640 | [symbol] | 0 |
| [symbol] | | | | [symbol] | | | | [symbol] | 0 | 0 | 757 | 0 | 334 | [symbol] | 221 | 0 |
| [symbol] | | | | [symbol] | | | | [symbol] | 0 | 0 | [symbol] | 1027 | [symbol] | 1832 | 231 | 0 |
| [symbol] | | | | [symbol] | | | | [symbol] | 0 | 767 | 45452 | 11547 | [symbol] | 1451 | [symbol] | [symbol] |
| [symbol] | | | | [symbol] | | | | [symbol] | 17 | 0 | 13847 | [symbol] | [symbol] | 10227 | 1871 | [symbol] |
| U | | | | 341 | | | | [symbol] | 134 | 154 | 0 | 0 | 1433 | 0 | 0 | [symbol] |
| [symbol] | | | | 342 | | | | [symbol] | 0 | 167 | [symbol] | [symbol] | [symbol] | [symbol] | 0 | [symbol] |
| P [symbol] | | | | 343 | | | | [symbol] | 0 | 2043 | 1731 | 174 | [symbol] | [symbol] | 29 | 422 |
| HCC [symbol] | | | | 3 [symbol] | | | | 177 [symbol] | 0 | [symbol] | 1731 | 774 | [symbol] | [symbol] 73 | 29 | 4 [symbol] |
| [symbol] | | | | 345 | | | | 16302 | 0 | [symbol] | 13 [symbol] | [symbol] | 2 [symbol] | 4 [symbol] | 1234 | [symbol] |
| HT192 | | | | 3 [symbol] | | | | 4 [symbol] | [symbol] 7 | [symbol] | 2 [symbol] 71 | 17 [symbol] | [symbol] | 14 [symbol] | 0 | [symbol] |
| COLO 205 | | | | 247 | | | | 12549 | 0 | 0 | 23 [symbol] | 0 | 109 | 0 | 757 | [symbol] |
| HT210 | | | | 345 | | | | 2 [symbol] 25 | 0 | 0 | 3 [symbol] | [symbol] 440 | [symbol] | 0 | 9 | 0 |
| [symbol] 72 | | | | 349 | | | | 21 [symbol] | 0 | 7 [symbol] | 217 [symbol] | 77 [symbol] | 2 [symbol] | 0 | [symbol] 31 | [symbol] |
| HT [symbol] 91 | | | | 350 | | | | [symbol] | [symbol] | [symbol] 70 | [symbol] 32 | [symbol] 52 | 43 | [symbol] | 511 | 27 [symbol] |
| [symbol] | | | | 351 | | | | 23 [symbol] | 0 | [symbol] | [symbol] 15 | 0 | 152 | [symbol] 50 | [symbol] | 0 |
| HT [symbol] | | | | 352 | | | | [symbol] 25 | 4 [symbol] | 0 | [symbol] | 19 [symbol] | 20 [symbol] | 0 | 0 | 0 |
| [symbol] | | | | 353 | | | | 23 [symbol] | 0 | 0 | 1917 | 237 | 205 [symbol] | 212 | 43 [symbol] | 0 |
| T [symbol] | | | | 3 [symbol] | | | | 42 [symbol] | 204 | [symbol] | 22002 | 5342 | [symbol] | 2400 | 23 | [symbol] 78 |
| [symbol] | | | | 357 | | | | 74434 | [symbol] 51 | [symbol] 7 | 7 [symbol] | 110 [symbol] | 3122 [symbol] | [symbol] 343 | 17 [symbol] | 27 [symbol] |
| [symbol] T | | | | 35 [symbol] | | | | 23715 | 71 | 2397 | 15 [symbol] | [symbol] | 141 [symbol] | 42 [symbol] | 372 | 0 |
| HT213 | | | 50 | | | | | 41730 | 0 | [symbol] | [symbol] | 12170 | 2 [symbol] | 0 | 0 | 0 |
| HT [symbol] | | | 52 | | | | | [symbol] 14 | [symbol] | 0 | [symbol] 90 | 3 [symbol] | 2 [symbol] | [symbol] | 0 | 0 |
| HT [symbol] | | | 54 | | | | | 27801 | 504 | 2 [symbol] | 1 [symbol] | 0 | 0 | 104 | 5 [symbol] | 143 |
| HT [symbol] | | | 5 [symbol] | | | | | 377 [symbol] 0 | 0 | 0 | 7 [symbol] | 12374 | 2 [symbol] | 9 | [symbol] | [symbol] |
| HT [symbol] | | | [symbol] | | | | | [symbol] 17 | 0 | 0 | 354 | [symbol] 4 | 0 | [symbol] | 141 | 0 |
| HT170 | | | [symbol] | | | | | [symbol] | 7 [symbol] | 0 | 73 [symbol] | 5 [symbol] | [symbol] 7 | [symbol] 07 | [symbol] 20 | 0 |
| HT172 | | | 62 | | | | | 313 [symbol] | [symbol] | 0 | [symbol] 71 | 3457 | 0 | 0 | [symbol] 34 | 357 |
| HT13 [symbol] | | | 63 | | | | | 41 [symbol] | 0 | 245 | 0 | 170 [symbol] | 0 | 171 | 0 | 0 |
| HT17 [symbol] | | | 64 | | | | | 37107 | 0 | 0 | 2 [symbol] | 1 [symbol] | 7 [symbol] | 0 | 443 | 45 |
| HT [symbol] | | | 65 | | | | | 32270 | 0 | 0 | 0 | 1173 [symbol] | 0 | [symbol] | 0 | 1 [symbol] |
| HT [symbol] | | | 57 | | | | | 24571 | [symbol] | 0 | [symbol] | 3 [symbol] | [symbol] | 73 | [symbol] | 27 |
| HT [symbol] | | | 57 | | | | | [symbol] | 0 | 0 | [symbol] | 1182 | [symbol] | 0 | [symbol] | 0 |
| HT [symbol] | | | 54 | | | | | [symbol] | 247 | 0 | [symbol] | 25 | [symbol] | 725 | [symbol] | 0 |
| HT143 | | | [symbol] | | | | | [symbol] | 0 | 475 | [symbol] | 0 | 0 | 0 | 0 | 247 |
| HT [symbol] | | | 70 | | | | | [symbol] | 135 | 0 | 435 | 5171 | 0 | 0 | [symbol] | 19 |
| HT [symbol] | | | 71 | | | | | 27515 | [symbol] | 0 | 7547 | [symbol] | 0 | 0 | 231 | [symbol] |
| HT [symbol] | | | 72 | | | | | [symbol] | 0 | 0 | [symbol] | [symbol] | 423 | 0 | [symbol] | 31 |
| HT302 | | | 73 | | | | | [symbol] | 0 | 0 | [symbol] | [symbol] | [symbol] | 0 | [symbol] | 0 |
| HT314 | | | 74 | | | | | [symbol] | 475 | 0 | 131 | [symbol] | [symbol] | 0 | 0 | 0 |
| HT317 | | | 76 | | | | | [symbol] | [symbol] | 0 | [symbol] | [symbol] | [symbol] | 0 | [symbol] | 0 |
| [symbol] | | | 77 | | | | | [symbol] | 0 | 0 | 142 | [symbol] | 0 | [symbol] | 0 | 0 |
| HT323 | | | 79 | | | | | [symbol] | [symbol] | 534 | 0 | 12917 | [symbol] | 0 | 0 | 0 |
| HT327 | | | 80 | | | | | [symbol] | [symbol] | 0 | 7 [symbol] | [symbol] | [symbol] | [symbol] | [symbol] | 0 |
| HT [symbol] | | | 82 | | | | | [symbol] | 0 | 017 [symbol] | 0 | [symbol] | [symbol] | [symbol] | [symbol] | [symbol] |
| HT [symbol] | | | 85 | | | | | [symbol] | 209 | 0 | 0 | [symbol] | 0 | 103 | [symbol] | [symbol] |
| HT343 | | | 87 | | | | | [symbol] | [symbol] | 0 | [symbol] | [symbol] | 0 | 126 | [symbol] | [symbol] |
| HT311 | | | 170 | | | | | [symbol] | [symbol] | 0 | 454 | [symbol] | [symbol] | 0 | 0 | 114 |
| HT [symbol] | | | 183 | | | | | [symbol] | 0 | 0 | 0 | 4527 | [symbol] | [symbol] | [symbol] | 0 |
| HT540 | | | [symbol] | | | | | [symbol] | 0 | 0 | 0 | [symbol] | 0 | 0 | [symbol] | [symbol] |
| HT251 | | | [symbol] | | | | | [symbol] | 0 | 0 | 0 | [symbol] | [symbol] | 0 | [symbol] | 0 |
| HT372 | | | 191 | | | | | 43132 | 0 | 0 | [symbol] | [symbol] | [symbol] | 0 | [symbol] | [symbol] |
| [symbol] | | | 20 [symbol] | | | | | 34117 | 0 | 154 | 1 | 0 | 0 | 0 | 0 | [symbol] |
| HT [symbol] | | | 21 [symbol] | | | | | [symbol] | 205 | 2 [symbol] | 1107 | [symbol] | 0 | 0 | 344 | 0 |
| HT207 | | | 217 | | | | | 174 [symbol] | 54 | 0 | 252 | [symbol] | [symbol] | 0 | 0 | 142 |
| HT [symbol] | | | 224 | | | | | [symbol] | 0 | 0 | [symbol] | [symbol] | [symbol] | [symbol] | 0 | [symbol] |
| HT379 | | | 225 | | | | | [symbol] | 0 | 0 | [symbol] | [symbol] | 0 | 0 | 0 | 0 |
| HT371 | | | 27 [symbol] | | | | | [symbol] | 0 | 1 | 0 | [symbol] | 254 | 2 | [symbol] | [symbol] |
| HT377 | | | 2 [symbol] | | | | | [symbol] | 423 | [symbol] | 0 | [symbol] | 0 | 0 | [symbol] | 0 |
| HT342 | | | [symbol] | | | | | [symbol] | 0 | 0 | [symbol] | [symbol] | 0 | 327 | 427 | 0 |
| [symbol] | | | [symbol] | | | | | [symbol] | [symbol] | 0 | 0 | [symbol] | 0 | 0 | 0 | 0 |
| HT334 | | | [symbol] | | | | | [symbol] | [symbol] | 0 | [symbol] | [symbol] | 0 | 0 | 0 | 0 |
| HT [symbol] | | | 301 | | | | | [symbol] | [symbol] | 0 | [symbol] | [symbol] | 145 | [symbol] | 221 | 0 |
| HT [symbol] | | | 3 [symbol] | | | | | [symbol] | 253 | 0 | [symbol] | [symbol] | 0 | [symbol] | 0 | 0 |
| HT [symbol] | | | 317 | | | | | [symbol] | 449 | 115 | [symbol] | [symbol] | 0 | [symbol] | 754 | [symbol] |
| HT312 | | | 318 | | | | | [symbol] | 0 | 0 | [symbol] | [symbol] | [symbol] | [symbol] | 379 | 0 |
| HT [symbol] | | | 224 | | | | | [symbol] | 0 | 522 | [symbol] | 0 | [symbol] | [symbol] | [symbol] | 0 |
| HT [symbol] | | | [symbol] | | | | | [symbol] | [symbol] | 0 | [symbol] | [symbol] | 0 | [symbol] | [symbol] | 0 |
| HT [symbol] | | | 3 [symbol] | | | | | 57 [symbol] | 0 | 0 | [symbol] | [symbol] | [symbol] | 233 | [symbol] | 0 |
| HT [symbol] | [symbol] | | | | | | | 28111 | 54 | [symbol] | [symbol] | [symbol] | 91 | [symbol] | [symbol] | 0 |
| HT [symbol] | 1 [symbol] 1 | | | | | | | [symbol] | 0 | [symbol] | [symbol] | [symbol] | 0 | [symbol] | [symbol] | 740 |
| HT [symbol] | [symbol] | | | | | | | [symbol] | [symbol] | [symbol] | [symbol] | [symbol] | [symbol] | 11425 | [symbol] | [symbol] |
| [symbol] | 157 | | | | | | | 47222 | 712 | [symbol] | [symbol] | [symbol] | [symbol] | [symbol] | [symbol] | 412 |
| [symbol] -1 | | | | | | | | [symbol] | 579 | 729 | 0 | 0 | 0 | 0 | 0 | 0 |
| [symbol] -4 | | | | | | | | 23439 | 748 | 374 | 0 | 0 | 0 | 0 | 0 | 0 |
| [symbol] -4 | | | | | | | | 44333 | 730 | 134 | 0 | 0 | 0 | 0 | 0 | 0 |
| [symbol] -11 | | | | | | | | [symbol] | 9 | [symbol] | 0 | 0 | 0 | 0 | 0 | 0 |
| [symbol] -12 | | | | | | | | [symbol] 743 | 8 | [symbol] | 0 | 0 | 0 | 0 | 0 | 0 |
| [symbol] -14 | | | | | | | | 20110 | 294 | 311 | 0 | 0 | 0 | 0 | 0 | 0 |
| [symbol] -1 | | | | | | | | 7 [symbol] 4 | [symbol] | 23 | 0 | 0 | 0 | 0 | 0 | 0 |
| [symbol] -2 | | | | | | | | 3130 | [symbol] 1 | 34 | 0 | 0 | 0 | 0 | 0 | 0 |
| [symbol] -3 | | | | | | | | [symbol] | 1703 | [symbol] | 0 | 0 | 0 | 0 | 0 | 0 |
| [symbol] -4 | | | | | | | | [symbol] 2 | 334 | 0 | 0 | 0 | 0 | 0 | 0 | 0 |
| [symbol] -5 | | | | | | | | [symbol] | 0 | 51 | 0 | 0 | 0 | 0 | 0 | 0 |
| [symbol] -6 | | | | | | | | 32 [symbol] | 20 | 0 | 0 | 0 | 0 | 0 | 0 | 0 |
| [symbol] -8 | | | | | | | wt | 19 [symbol] | 23 | [symbol] | 0 | 0 | 0 | 0 | 0 | 0 |
| EXVX-7 | | | | | | | [symbol] | [symbol] | 212 | 0 | 0 | 0 | 0 | 0 | 0 | 0 |
| HCT-118-7 | | | | | | | wt | 6 | 723 | 0 | 0 | 0 | 0 | 0 | 0 | 0 |
| HCT-114-6 | | | | | | | wt | 771 | 122 | [symbol] | 0 | 0 | 0 | 0 | 0 | 0 |
| [symbol] 29-1 | | | | | | | [symbol] | 310 | 0 | 144 | 0 | 0 | 0 | 0 | 0 | 0 |
| [symbol] 29-7 | | | | | | | [symbol] | 0 | 0 | [symbol] | 0 | 0 | 0 | 0 | 0 | 0 |
| [symbol] 29-8 | | | | | | | [symbol] | 0 | 0 | [symbol] | 0 | 0 | 0 | 0 | 0 | 0 |
| SF630-7 | | | | | | | wt | 1 [symbol] 7 | 837 | [symbol] | 0 | 0 | 0 | 0 | 0 | 0 |
| SF630-8 | | | | | | | wt | 3330 | 7 | 130 | 0 | 0 | 0 | 0 | 0 | 0 |
| SF- [symbol] | | | | | | | [symbol] | 17073 | [symbol] 3 | 0 | 0 | 0 | 0 | 0 | 0 | 0 |
| SF- [symbol] | | | | | | | [symbol] | 18222 | 32 | 110 | 0 | 0 | 0 | 0 | 0 | 0 |
| OVCAR-4-7 | | | | | | | wt | 1233 | 29 | 0 | 0 | 0 | 0 | 0 | 0 | 0 |
| OVCAR-4-8 | | | | | | | wt | 12784 | 4 | 171 | 0 | 0 | 0 | 0 | 0 | 0 |
| OVCAR-5-7 | | | | | | | [symbol] | 4722 | 0 | 0 | 0 | 0 | 0 | 0 | 0 | 0 |
| OVCAR-6-7 | | | | | | | [symbol] | 3132 | 0 | 4 | 0 | 0 | 0 | 0 | 0 | 0 |
| MCF-7-8 | | | | | | | wt | 3075 | 440 | 0 | 0 | 0 | 0 | 0 | 0 | 0 |
| [symbol] -8 | | | | | | | [symbol] | 630 | 0 | 0 | 0 | 0 | 0 | 0 | 0 | 0 |
| [symbol] | | | | | | | [symbol] | 1007 | [symbol] | 0 | 0 | 0 | 0 | 0 | 0 | 0 |
| SW400-7 | | | | | | | [symbol] | 1922 | 717 | 22 | 0 | 0 | 0 | 0 | 0 | 0 |
| SW400- [symbol] | | | | | | | [symbol] | 417 | 4 | 72 | 0 | 0 | 0 | 0 | 0 | 0 |
| HT200-8 | | | | | | | [symbol] | 0 | 142 | 10 | 0 | 0 | 0 | 0 | 0 | 0 |
| C32A-7 | | | | | | | [symbol] | 0 | 230 | 4 | 0 | 0 | 0 | 0 | 0 | 0 |
| C32A-8 | | | | | | | [symbol] | 444 | 0 | 0 | 0 | 0 | 0 | 0 | 0 | 0 |
| V2O3-7 | | | | | | | [symbol] | 20013 | 142 | 84 | 0 | 0 | 0 | 0 | 0 | 0 |
| V2O4-8 | | | | | | | [symbol] | 4766 | 416 | 0 | 0 | 0 | 0 | 0 | 0 | 0 |
| [symbol] -7 | | | | | | | wt | 1383 | 0 | 0 | 0 | 0 | 0 | 0 | 0 | 0 |
| [symbol] -8 | | | | | | | wt | 512 | 326 | 0 | 0 | 0 | 0 | 0 | 0 | 0 |
| WI34-8 | | | | | | | wt | 1934 | 41 | 0 | 0 | 0 | 0 | 0 | 0 | 0 |
| [symbol] | | | | | | | | 0 | 0 | 0 | 70 | 5790 | 2685 | 304 | [symbol] | [symbol] |
| [symbol] | | | | | | | | 340 | 0 | 0 | 134 | 1830 | 233 | 6 | 317 | [symbol] |
| [symbol] | | | | | | 84 | | 442 | 0 | 0 | 189 | 5674 | 0 | [symbol] | 0 | 12425 |
| WT374 | | | | | | | | 0 | 0 | 0 | 801 | 4679 | 0 | 0 | 7 | [symbol] |
| WT374 | | | | | | | | 0 | 0 | 34 | 0 | 4412 | 215 | 0 | 0 | 2034 |
| WT330 | | | | | | | | 10732 | 0 | 0 | 0 | 5687 | 5257 | 31 | 154 | [symbol] |
| WT330 | | | | | | | | 0 | 0 | 0 | 0 | 6143 | 163 | 0 | 0 | 34587 |
| [symbol] -3 | | | | | | 173 | | 53 | 0 | 7 | 0 | 3031 | 490 | 325 | 297 | 1816 |
| [symbol] -5 | | | | | | 175 | | 0 | 0 | 55 | 34 | 1424 | 643 | 204 | 0 | 53 |
| [symbol] -9 | | | | | | 177 | | 0 | 0 | 7 | 17 | 2354 | 224 | 0 | 0 | 1290 |
| [symbol] Feb. 25, 2002 [symbol] | | | | | | | | 0 | 0 | 23 | 13 | 3475 | 0 | 112 | 0 | 195 |
| [symbol] -10 | | | | | | 237 | | 0 | 0 | 10 | 123 | 4083 | 130 | 40 | 291 | 3522 |
| WTA-10 | | | | | | | | 0 | 0 | 9 | 0 | 7631 | 0 | 431 | 660 | 1424 |
| [symbol] Mar. 31, 2002 [symbol] | | | | | | | | 0 | 0 | 30 | 23 | 4848 | 0 | 0 | 204 | 603 |
| [symbol] | | | | | | | | 0 | 0 | 0 | 91 | 2003 | 0 | 0 | 0 | [symbol] |
| [symbol] | | | | | | | | 915 | 0 | 0 | 0 | 3067 | 234 | [symbol] | 0 | 2243 |
| [symbol] | | | | | | | | 0 | 0 | 0 | [symbol] | 1148 | 0 | 175 | 0 | 1566 |
| [symbol] | | | | | | | | 0 | 0 | 52 | 0 | 2629 | 0 | 257 | 0 | 3872 |
| HCT-114-3 | | | | | | | wt | 394 | 22 | 2 | 0 | 0 | 0 | 0 | 0 | 0 |
| HCT-114-4 | | | | | | | wt | 434 | 1211 | 0 | 0 | 0 | 0 | 0 | 0 | 0 |
| HCT-114-5 | | | | | | | wt | 127 | 294 | 24 | 0 | 0 | 0 | 0 | 0 | 0 |
| HCT-114-6 | | | | | | | wt | 1178 | 9 | 9 | 0 | 0 | 0 | 0 | 0 | 0 |
| [symbol] | | | | | | | wt | 0 | 0 | 4 | 0 | 0 | 0 | 0 | 0 | 0 |
| WT [symbol] -3 | | | | | | | [symbol] | [symbol] | 0 | 347 | 0 | 0 | 0 | 0 | 0 | 0 |
| EXVX-6 | | | | | | | [symbol] | [symbol] | 0 | 0 | 0 | 0 | 0 | 0 | 0 | 0 |
| HT29-4 | | | | | | | [symbol] | 0 | 17 | 24 | 0 | 0 | 0 | 0 | 0 | 0 |
| HT29-5 | | | | | | | [symbol] | 1078 | 6 | 244 | 0 | 0 | 0 | 0 | 0 | 0 |
| HT29-6 | | | | | | | [symbol] | 0 | 0 | 52 | 0 | 0 | 0 | 0 | 0 | 0 |
| OVCAR-4-2 | | | | | | | wt | 5253 | 10 [symbol] | 30 | 0 | 0 | 0 | 0 | 0 | 0 |
| OVCAR-4-4 | | | | | | | wt | 0 | 0 | 0 | 0 | 0 | 0 | 0 | 0 | 0 |
| OVCAR-4-5 | | | | | | | wt | 2000 | 271 | 0 | 0 | 0 | 0 | 0 | 0 | 0 |
| OVCAR-4-6 | | | | | | | wt | 0 | 0 | 32 | 0 | 0 | 0 | 0 | 0 | 0 |
| [symbol] -3 | | | | | | | wt | 4442 | 146 | 140 | 0 | 0 | 0 | 0 | 0 | 0 |
| [symbol] -4 | | | | | | | wt | 3290 | 291 | 32 | 0 | 0 | 0 | 0 | 0 | 0 |
| [symbol] -5 | | | | | | | wt | 591 | 0 | 30 | 0 | 0 | 0 | 0 | 0 | 0 |
| [symbol] -6 | | | | | | | wt | 1200 | 312 | 0 | 0 | 0 | 0 | 0 | 0 | 0 |
| OVCAR-6-3 | | | | | | | [symbol] | 2344 | 0 | 116 | 0 | 0 | 0 | 0 | 0 | 0 |
| OVCAR-6-4 | | | | | | | [symbol] | 9 | 0 | 31 | 0 | 0 | 0 | 0 | 0 | 0 |
| OVCAR-6-5 | | | | | | | [symbol] | 241 | 0 | 9 | 0 | 0 | 0 | 0 | 0 | 0 |
| OVCAR-6-6 | | | | | | | [symbol] | 99 | 0 | 124 | 0 | 0 | 0 | 0 | 0 | 0 |
| MCF-7- [symbol] | | | | | | | w4 | 620 | [symbol] | [symbol] | 0 | 0 | 0 | 0 | 0 | 0 |
| [symbol] - | | | | | | | MPV E4 | 1297 | 1627 | 0 | 0 | 0 | 0 | 0 | 0 | 0 |
| [symbol] -5 | | | | | | | [symbol] | 0 | 2 [symbol] | 9 | 0 | 0 | 0 | 0 | 0 | 0 |
| SW400-3 | | | | | | | [symbol] | 0 | 303 | 139 | 0 | 0 | 0 | 0 | 0 | 0 |
| SW400-4 | | | | | | | [symbol] | 1137 | [symbol] | 87 | 0 | 0 | 0 | 0 | 0 | 0 |
| SW400-5 | | | | | | | [symbol] | 3274 | 0 | 30 | 0 | 0 | 0 | 0 | 0 | 0 |
| SW400-6 | | | | | | | [symbol] | [symbol] | 726 | 0 | 0 | 0 | 0 | 0 | 0 | 0 |
| C33A-3 | | | | | | | [symbol] | [symbol] | 0 | 10 | 0 | 0 | 0 | 0 | 0 | 0 |
| C33A-4 | | | | | | | [symbol] | 4370 | 0 | 0 | 0 | 0 | 0 | 0 | 0 | 0 |
| C33A-5 | | | | | | | [symbol] | 0 | 876 | 20 | 0 | 0 | 0 | 0 | 0 | 0 |
| C33A-6 | | | | | | | [symbol] | [symbol] | 0 | 0 | 0 | 0 | 0 | 0 | 0 | 0 |
| [symbol] | | | | | | | wt | 0 | 0 | 60 | 0 | 0 | 0 | 0 | 0 | 0 |
| [symbol] -3 | | | | | | | [symbol] | 71007 | [symbol] 3 | 9 | 0 | 0 | 0 | 0 | 0 | 0 |
| [symbol] -4 | | | | | | | [symbol] | 4820 | 0 | 0 | 0 | 0 | 0 | 0 | 0 | 0 |
| [symbol] -5 | | | | | | | [symbol] | 7 [symbol] 1 | 147 | 17 | 0 | 0 | 0 | 0 | 0 | 0 |
| [symbol] -6 | | | | | | | [symbol] | 0 | 0 | 49 | 0 | 0 | 0 | 0 | 0 | 0 |
| WI24- [symbol] | | | | | | | wt | 2111 | 0 | 202 | 0 | 0 | 0 | 0 | 0 | 0 |
| [symbol] -3 | | | | | | | wt | 2002 | 0 | 57 | 0 | 0 | 0 | 0 | 0 | 0 |
| [symbol] -4 | | | | | | | wt | 93108 | 1352 | 240 | 0 | 0 | 0 | 0 | 0 | 0 |
| SF-200-3 | | | | | | | [symbol] | 12 [symbol] | 0 | 0 | 0 | 0 | 0 | 0 | 0 | 0 |
| SF-200-4 | | | | | | | [symbol] | 70341 | 1940 | 0 | 0 | 0 | 0 | 0 | 0 | 0 |
| SF-200-5 | | | | | | | [symbol] | 32200 | 292 | 0 | 0 | 0 | 0 | 0 | 0 | 0 |
| SF-200-6 | | | | | | | [symbol] | 0 | 2127 | 0 | 0 | 0 | 0 | 0 | 0 | 0 |
| [symbol] -17 | | | | | | | | 43184 | 820 | 790 | 0 | 0 | 0 | 0 | 0 | 0 |
| [symbol] -20 | | | | | | | | 5419 | 5443 | 71 | 0 | 0 | 0 | 0 | 0 | 0 |
| [symbol] -21 | | | | | | | | 6234 | 3043 | 129 | 0 | 0 | 0 | 0 | 0 | 0 |
| [symbol] -22 | | | | | | | | 70 [symbol] | 9867 | 300 | 0 | 0 | 0 | 0 | 0 | 0 |
| OVCAR- [symbol] | | | | | | | [symbol] | [symbol] 10 | 783 | 112 | 0 | 0 | 0 | 0 | 0 | 0 |
| [symbol] -10 | | | | | | | | 2794 | 1333 | 113 | 0 | 0 | 0 | 0 | 0 | 0 |
| [symbol] -11 | | | | | | | | [symbol] | 701 | 45 | 0 | 0 | 0 | 0 | 0 | 0 |
| [symbol] -12 | | | | | | | | [symbol] | 81 | 296 | 0 | 0 | 0 | 0 | 0 | 0 |
| [symbol] -13 | | | | | | | | 4834 | 133 | 1023 | 0 | 0 | 0 | 0 | 0 | 0 |
| [symbol] -14 | | | | | | | | 4337 | 0 | 891 | 0 | 0 | 0 | 0 | 0 | 0 |
| [symbol] -15 | | | | | | | | [symbol] | 0 | 1013 | 0 | 0 | 0 | 0 | 0 | 0 |
| [symbol] -16 | | | | | | | | 3477 | 2 | 0 | 0 | 0 | 0 | 0 | 0 | 0 |
| [symbol] -17 | | | | | | | | 1275 | 6 | 195 | 0 | 0 | 0 | 0 | 0 | 0 |
| [symbol] -18 | | | | | | | | 12 [symbol] | 3430 | 995 | 0 | 0 | 0 | 0 | 0 | 0 |
| [symbol] -19 | | | | | | | | 3 | 0 | 0 | 0 | 0 | 0 | 0 | 0 | 0 |
| [symbol] | | 1 | | | | | | 1179 | [symbol] | 13724 | 10216 | 11733 | 18738 | [symbol] | [symbol] | [symbol] |
| [symbol] | | 2 | | | | | | 1842 | [symbol] | [symbol] | [symbol] | [symbol] | [symbol] | [symbol] | [symbol] | [symbol] |
| [symbol] | | 3 | | | | | | [symbol] | [symbol] | [symbol] | [symbol] | [symbol] | [symbol] | [symbol] | [symbol] | [symbol] |
| [symbol] | | 4 | | | | | | 102 | [symbol] | [symbol] | [symbol] | [symbol] | [symbol] | [symbol] | [symbol] | [symbol] |
| [symbol] | | 5 | | | | | | 2734 | 23741 | [symbol] | [symbol] | 113857 | [symbol] | [symbol] | [symbol] | [symbol] |
| [symbol] | | 6 | | | | | | 2187 | [symbol] | [symbol] | [symbol] | [symbol] | [symbol] | [symbol] | [symbol] | [symbol] |
| [symbol] | | 7 | | | | | | [symbol] | [symbol] | [symbol] | [symbol] | [symbol] | [symbol] | [symbol] | [symbol] | [symbol] |
| [symbol] | | 8 | | | | | | 2102 | [symbol] | [symbol] | [symbol] | [symbol] | [symbol] | [symbol] | [symbol] | [symbol] |
| [symbol] | | 9 | | | | | | [symbol] | [symbol] | [symbol] | [symbol] | [symbol] | [symbol] | [symbol] | [symbol] | [symbol] |
| [symbol] | | 10 | | | | | | [symbol] | [symbol] | [symbol] | [symbol] | [symbol] | [symbol] | [symbol] | [symbol] | [symbol] |
| [symbol] | | 11 | | | | | | [symbol] | [symbol] | [symbol] | [symbol] | [symbol] | [symbol] | [symbol] | [symbol] | [symbol] |
| [symbol] | | 12 | | | | | | [symbol] | [symbol] | [symbol] | [symbol] | [symbol] | [symbol] | [symbol] | [symbol] | [symbol] |
| [symbol] | | 13 | | | | | | 1276 | [symbol] | [symbol] | [symbol] | [symbol] | [symbol] | [symbol] | [symbol] | [symbol] |
| [symbol] | | 14 | | | | | | [symbol] | [symbol] | [symbol] | [symbol] | [symbol] | [symbol] | [symbol] | 47817 | [symbol] |
| [symbol] | | 15 | | | | | | [symbol] | [symbol] | [symbol] | [symbol] | [symbol] | [symbol] | [symbol] | 23301 | [symbol] |
| [symbol] | | 16 | | | | | | 1234 | [symbol] | [symbol] | [symbol] | [symbol] | [symbol] | [symbol] | [symbol] | [symbol] |
| [symbol] | | 17 | | | | | | 2013 | [symbol] | [symbol] | [symbol] | [symbol] | [symbol] | [symbol] | [symbol] | [symbol] |
| [symbol] | | 18 | | | | | | [symbol] | [symbol] | [symbol] | [symbol] | [symbol] | [symbol] | [symbol] | 8412 | [symbol] |
| [symbol] | | 19 | | | | | | 2970 | [symbol] | [symbol] | [symbol] | [symbol] | [symbol] | [symbol] | [symbol] | [symbol] |
| [symbol] | | 20 | | | | | | 4795 | [symbol] | 23720 | [symbol] | [symbol] | [symbol] | [symbol] | [symbol] | [symbol] |
| [symbol] | | 21 | | | | | | [symbol] | [symbol] | 15471 | [symbol] | [symbol] | [symbol] | [symbol] | 1081 | [symbol] |
| [symbol] | | 22 | | | | | | 484 | [symbol] | 15711 | [symbol] | [symbol] | [symbol] | [symbol] | [symbol] | [symbol] |
| [symbol] | | 23 | | | | | | 1419 | [symbol] | 20778 | [symbol] | [symbol] | [symbol] | [symbol] | 4881 | [symbol] |
| [symbol] | | 24 | | | | | | [symbol] | [symbol] | 22132 | 2177 | [symbol] | [symbol] | [symbol] | [symbol] | [symbol] |
| [symbol] | | 25 | | | | | | 1876 | [symbol] | 10425 | [symbol] | [symbol] | [symbol] | [symbol] | [symbol] | [symbol] |
| [symbol] | | 26 | | | | | | 294 | [symbol] | 103115 | 6714 | [symbol] | [symbol] | [symbol] | 20107 | [symbol] |
| [symbol] | | 27 | | | | | | [symbol] | [symbol] | [symbol] | [symbol] | [symbol] | [symbol] | [symbol] | [symbol] | [symbol] |
| [symbol] | | 28 | | | | 28 | | 204 | [symbol] | 14425 | [symbol] | [symbol] | [symbol] | [symbol] | 0 | [symbol] |
| [symbol] | | 29 | | | | | | [symbol] | [symbol] | [symbol] | [symbol] | [symbol] | [symbol] | [symbol] | [symbol] | [symbol] |
| [symbol] | | 30 | | | | 30 | | 100 | [symbol] | [symbol] | [symbol] | [symbol] | [symbol] | [symbol] | 13265 | [symbol] |
| [symbol] | | 31 | | | | | | [symbol] | [symbol] | [symbol] | [symbol] | 0 | [symbol] | [symbol] | 21164 | [symbol] |
| [symbol] | | 32 | | | | | | [symbol] | [symbol] | [symbol] | 1729 | 34917 | [symbol] | [symbol] | 5423 | [symbol] |
| [symbol] | | 33 | | | | | | [symbol] | [symbol] | 41825 | $113 | 436 | [symbol] | [symbol] | [symbol] | [symbol] |
| [symbol] | | 34 | | | | | | 0 | [symbol] | [symbol] | 3353 | 290 | 18410 | [symbol] | 4218 | [symbol] |
| [symbol] | | 35 | | | | | | [symbol] | [symbol] | [symbol] | [symbol] | 434 | 17803 | [symbol] | [symbol] | [symbol] |
| [symbol] | | 36 | | | | | | 1743 | [symbol] | [symbol] | 0 | [symbol] | 5241 | [symbol] | 3205 | [symbol] |
| [symbol] | | 37 | | | | | | 621 | [symbol] | 12132 | [symbol] | 1096 | 7190 | [symbol] | [symbol] | [symbol] |
| [symbol] | | 38 | | | | | | 0 | [symbol] | [symbol] | [symbol] | 100 | 4538 | [symbol] | 30874 | [symbol] |
| [symbol] | | 39 | | | | | | [symbol] | [symbol] | [symbol] | 1071 | [symbol] | 14300 | [symbol] | 1733 | [symbol] |
| [symbol] | | 40 | | | | | | 1510 | [symbol] | 0 | [symbol] | 406 | 4578 | [symbol] | [symbol] | [symbol] |
| [symbol] | | 41 | | | | | | 927 | [symbol] | [symbol] | [symbol] | [symbol] | [symbol] | [symbol] | [symbol] | [symbol] |
| [symbol] | | 42 | | | | | | 1470 | [symbol] | 742 | 4500 | 0 | 4145 | [symbol] | [symbol] | [symbol] |
| [symbol] | | 43 | | | | | | [symbol] | [symbol] | [symbol] | 103 | 0 | [symbol] | [symbol] | 3474 | [symbol] |
| [symbol] | | 44 | | | | | | [symbol] | [symbol] | [symbol] | [symbol] | 130 | [symbol] | 2101 | 10038 | [symbol] |
| [symbol] | | | | | [symbol] | | | 0 | [symbol] | [symbol] | [symbol] | 0 | 13181 | 1814 | 501 | [symbol] |
| [symbol] | | | | | 383 | | | 0 | [symbol] | 752 | 4540 | 22 | [symbol] | 1702 | 0 | [symbol] |
| [symbol] | | | | | 361 | | | 451 | [symbol] | 0 | 70 | 5137 | 143 | [symbol] | 338 | [symbol] |
| [symbol] | | | | | 356 | 354 | | 290 | [symbol] | [symbol] | 131 | [symbol] | 171 | 14131 | 14830 | [symbol] |
| [symbol] | | | | | 354 | 354 | | [symbol] | [symbol] | 0 | 0 | [symbol] | 10447 | [symbol] | 2525 | [symbol] |
| [symbol] | | | | | 344 | | | 0 | [symbol] | [symbol] | [symbol] | 0 | [symbol] | 2304 | 1411 | [symbol] |
| [symbol] | | | | | 342 | 334 | | 0 | [symbol] | 0 | 21 | 257 | 428 | [symbol] | 443 | [symbol] |
| [symbol] | | | | | 334 | | | [symbol] | [symbol] | 1144 | [symbol] | 7 | 129 | 4204 | 274 | [symbol] |
| [symbol] | | | | | 332 | | | 153 | [symbol] | [symbol] | 175 | [symbol] | 701 | [symbol] | 0 | [symbol] |
| [symbol] | | | | | 330 | | | 0 | [symbol] | 0 | 0 | [symbol] | [symbol] | [symbol] | 0 | [symbol] |
| [symbol] | | | | | 328 | | | 254 | [symbol] | 0 | [symbol] | 1 | 34 | [symbol] | 3700 | [symbol] |
| [symbol] | | | | | 327 | | | 1213 | [symbol] | 0 | 0 | 5123 | [symbol] | [symbol] | [symbol] | [symbol] |
| [symbol] | | | | | [symbol] | | | [symbol] | [symbol] | 125 | [symbol] | 1 | 1023 | [symbol] | [symbol] | [symbol] |
| [symbol] | | | | | 321 | | | 0 | [symbol] | 0 | 87 | 214 | 4823 | [symbol] | 152 | [symbol] |
| [symbol] | | | | | 320 | | | 1317 | [symbol] | 0 | [symbol] | 1631 | 1315 | 2550 | [symbol] | [symbol] |
| [symbol] | | | | | 318 | | | 0 | [symbol] | [symbol] | 0 | [symbol] | 5813 | 4217 | 30278 | [symbol] |
| [symbol] | | | | | 318 | | | 0 | [symbol] | 0 | [symbol] | 151 | [symbol] | 11200 | 140783 | [symbol] |
| [symbol] | | | | | 314 | | | 1 | [symbol] | 72 | 1232 | 0 | 12222 | 2519 | [symbol] | [symbol] |
| [symbol] | | | | | 311 | | | 0 | [symbol] | [symbol] | [symbol] | 120 | 10332 | 2442 | 4572 | [symbol] |
| [symbol] | | | | | [symbol] | | | 0 | [symbol] | [symbol] | [symbol] | [symbol] | 2734 | [symbol] | 5044 | [symbol] |
| [symbol] | | | | | [symbol] | | | [symbol] | [symbol] | 0 | 0 | 0 | 1822 | [symbol] | 2133 | [symbol] |
| [symbol] | | | | | [symbol] | | | 0 | [symbol] | 0 | 0 | 0 | 1127 | 4341 | [symbol] | [symbol] |
| [symbol] | | | | | [symbol] | | | 0 | [symbol] | 0 | 233 | 220 | [symbol] | [symbol] | [symbol] | [symbol] |
| [symbol] | | | | | [symbol] | | | 650 | [symbol] | 0 | 0 | 0 | [symbol] | [symbol] | [symbol] | [symbol] |
| [symbol] | | | | | [symbol] | | | 0 | [symbol] | 0 | [symbol] | 0 | [symbol] | [symbol] | [symbol] | [symbol] |
| [symbol] | | | | | [symbol] | | | 104 | [symbol] | [symbol] | 114 | [symbol] | [symbol] | 4436 | 18214 | [symbol] |
| [symbol] | | | | | 302 | | | 1973 | [symbol] | 0 | 45 | 1802 | [symbol] | [symbol] | [symbol] | [symbol] |
| [symbol] | | | | | 297 | | | 1947 | [symbol] | 1050 | 487 | [symbol] | [symbol] | 6236 | 29075 | [symbol] |
| [symbol] | | | | | 296 | | | [symbol] | [symbol] | [symbol] | [symbol] | 117 | 19310 | 2770 | 13181 | [symbol] |
| [symbol] | | | | | 294 | | | 205 | [symbol] | 0 | [symbol] | $1208 | 8744 | 7973 | [symbol] | [symbol] |
| [symbol] | | | | | 292 | | | [symbol] | [symbol] | 0 | 330 | 70 | [symbol] | 2923 | 11584 | [symbol] |
| [symbol] | | | | | 290 | | | 2157 | [symbol] | 0 | 137 | [symbol] | 10727 | [symbol] | 8383 | [symbol] |
| [symbol] | | | | | 279 | | | 0 | [symbol] | 0 | 52 | 0 | 1241 | [symbol] | 23427 | [symbol] |
| [symbol] | | | | | 277 | | | 0 | [symbol] | 0 | 0 | 0 | 11134 | 2195 | [symbol] | [symbol] |
| [symbol] | | | | | 275 | 275 | | [symbol] | [symbol] | [symbol] | [symbol] | 0 | 0 | [symbol] | 675 | [symbol] |
| [symbol] | | | | | [symbol] | | | 0 | [symbol] | 0 | 0 | 247 | [symbol] | 3137 | 0 | [symbol] |
| [symbol] | | | | | [symbol] | | | 2212 | [symbol] | [symbol] | 114 | [symbol] | [symbol] | [symbol] | 407 | [symbol] |
| [symbol] | | | | | [symbol] | 230 | | [symbol] | [symbol] | 2131 | 152 | 0 | 0 | 4200 | 2147 | [symbol] |
| [symbol] | | | | | 235 | 235 | | 0 | [symbol] | 0 | 0 | 0 | 1078 | 2783 | 10003 | [symbol] |
| [symbol] | | | | | 234 | | | 0 | [symbol] | 1494 | 0 | 0 | [symbol] | 3430 | 1723 | [symbol] |
| [symbol] | | | | | 233 | 233 | | 3431 | [symbol] | [symbol] | 420 | 227 | 2750 | [symbol] | 403 | [symbol] |
| [symbol] | | | | | 231 | 231 | | [symbol] | [symbol] | 1405 | 116 | 0 | 17 | 2754 | 4357 | [symbol] |
| [symbol] | | | | | 229 | 229 | | 197 | [symbol] | 1572 | 17 | 23 | [symbol] | [symbol] | [symbol] | [symbol] |
| [symbol] | | | | | 227 | 227 | | [symbol] | [symbol] | 54 | 0 | [symbol] | 1455 | 2494 | 2380 | [symbol] |
| [symbol] | | | | | 222 | | | 250 | [symbol] | 55 | 145 | 322 | 1082 | [symbol] | [symbol] | [symbol] |
| [symbol] | | | | | 215 | | | 0 | [symbol] | 229 | 17 | 29 | 1520 | 3087 | 343 | [symbol] |
| [symbol] | | | | | 214 | | | 0 | [symbol] | 0 | 0 | 0 | 0 | [symbol] | 1770 | [symbol] |
| [symbol] | | | | | 213 | | | 0 | [symbol] | 0 | 0 | [symbol] | [symbol] | 2202 | [symbol] | [symbol] |
| [symbol] | | | | | 212 | | | 130 | [symbol] | 0 | 0 | 153 | [symbol] | [symbol] | 480 | [symbol] |
| [symbol] | | | | | 211 | 211 | | 135 | [symbol] | 412 | 0 | 117 | 454 | 2312 | 7793 | [symbol] |
| [symbol] | | | | | 210 | | | 0 | [symbol] | 40 | 0 | 220 | [symbol] | [symbol] | 1 | [symbol] |
| [symbol] | | | | | 209 | | | 170 | [symbol] | 0 | [symbol] | [symbol] | 0 | 2108 | 4178 | [symbol] |
| [symbol] | | | | | 205 | | | [symbol] | [symbol] | 0 | 4 | 0 | 483 | 1052 | 0 | [symbol] |
| [symbol] | | | | | 203 | | | [symbol] | [symbol] | 0 | 13 | [symbol] | 3 | [symbol] | 238 | [symbol] |
| [symbol] | | | | | 201 | | | 0 | [symbol] | 0 | 0 | 0 | 180 | 2429 | 124 | [symbol] |
| [symbol] | | | | | 180 | | | 385 | [symbol] | 0 | [symbol] | [symbol] | 0 | 2744 | 304 | [symbol] |
| [symbol] | | | | | 187 | | | 0 | [symbol] | 0 | [symbol] | 411 | 2172 | 2792 | 380 | [symbol] |
| [symbol] | | | | | 194 | | | [symbol] | [symbol] | 0 | 132 | 22 | 534 | 1518 | 2052 | [symbol] |
| [symbol] | | | | | 193 | | | 0 | [symbol] | 0 | 0 | 25 | 294 | 1254 | 42 | [symbol] |
| [symbol] | | | | | [symbol] | | | 0 | [symbol] | 0 | 204 | 0 | [symbol] | [symbol] | 2652 | [symbol] |
| [symbol] | | | | | [symbol] | | | 0 | [symbol] | 0 | 0 | 0 | [symbol] | 830 | 72 | [symbol] |
| [symbol] | | | | | 50 | | | 0 | [symbol] | 0 | 158 | 84 | [symbol] | 2001 | 170 | 34783 |
| [symbol] | | | | | 57 | | | 0 | [symbol] | 43 | [symbol] | 0 | 2379 | 2154 | 5507 | 175192 |
| [symbol] | | | | | [symbol] | | | 1173 | [symbol] | [symbol] | 0 | [symbol] | 1020 | 2135 | 3445 | [symbol] |
| [symbol] | | | | | 53 | | | 273 | [symbol] | [symbol] | 0 | 0 | 0 | 777 | [symbol] | [symbol] |
| [symbol] | | | | | [symbol] | | | 0 | [symbol] | 0 | [symbol] | 176 | 0 | 1157 | 822 | [symbol] |
| [symbol] | | | | | 40 | | | 380 | [symbol] | [symbol] | [symbol] | 0 | [symbol] | [symbol] | [symbol] | 23343 |
| HELA [symbol] | | | | 79 | | | | [symbol] | [symbol] | 0 | [symbol] | [symbol] | [symbol] | 2750 | 5247 | 95108 |
| HELA [symbol] | | | | 81 | | | | [symbol] | [symbol] | [symbol] | [symbol] | 0 | 781 | 2330 | 21234 | [symbol] |
| HELA [symbol] | | | | [symbol] | | | | [symbol] | [symbol] | 0 | 50 | 203 | 1276 | 2295 | [symbol] | [symbol] |
| HELA [symbol] | | | | [symbol] | | | | [symbol] | [symbol] | 0 | [symbol] | 80 | 2720 | 1297 | 4512 | [symbol] |
| HELA [symbol] | | | | [symbol] | | | | [symbol] | [symbol] | [symbol] | 0 | 81 | 0 | 1843 | 4070 | 23775 |
| HELA [symbol] | | | | [symbol] | | | | [symbol] | [symbol] | 0 | [symbol] | 82 | [symbol] | 1744 | 4304 | [symbol] |
| HELA [symbol] | | | | [symbol] | | | | [symbol] | [symbol] | 0 | [symbol] | 90 | [symbol] | 1781 | 3048 | [symbol] |
| HELA [symbol] | | | | [symbol] | | | | [symbol] | [symbol] | 0 | 195 | 192 | 0 | [symbol] | 7823 | 78133 |
| HELA [symbol] | | | | [symbol] | | | | [symbol] | [symbol] | 0 | 0 | 0 | [symbol] | 1400 | 2544 | [symbol] |
| [symbol] | | | | [symbol] | | | | [symbol] | [symbol] | 0 | [symbol] | [symbol] | [symbol] | 4158 | 1829 | [symbol] |
| [symbol] | | | | 148 | | | | [symbol] | [symbol] | 0 | 703 | [symbol] | 432 | 2184 | [symbol] | [symbol] |
| [symbol] | | | | 150 | | | | 0 | [symbol] | 0 | 275 | 0 | 378 | 1871 | 13323 | 24403 |
| [symbol] | | | | 152 | | | | [symbol] | [symbol] | 0 | [symbol] | 0 | 1114 | 2329 | [symbol] | [symbol] |
| [symbol] | | | | 154 | | | | [symbol] | [symbol] | 0 | [symbol] | 40 | 2304 | 3295 | 17173 | 20001 |
| [symbol] | | | | 154 | | | | 0 | [symbol] | 432 | 233 | 0 | 1122 | 1851 | [symbol] | 14348 |
| [symbol] | | | | [symbol] | | | | 0 | [symbol] | 0 | 87 | 434 | 732 | 254 | 2043 | [symbol] |
| CCRF [symbol] | | | | 180 | | | | [symbol] | [symbol] | 0 | 0 | 128 | 821 | [symbol] | 303 | [symbol] |
| [symbol] 145 | | | | 152 | | | | 0 | [symbol] | 0 | [symbol] | 40 | [symbol] | 1957 | 13480 | 18145 |
| MC7 [symbol] | | | | 184 | | | | 0 | [symbol] | 0 | 245 | 0 | 471 | 1324 | 19125 | 12384 |
| [symbol] 5737 | 154 | | | | | | | [symbol] | 143 | 111814 | 7208 | [symbol] | [symbol] | 8343 | 0 | [symbol] |
| MCF- [symbol] | 153 | | | | | | | [symbol] | [symbol] | 70411 | 721 | 387 | 0 | [symbol] | 0 | 838 |
| MCF 7 | 151 | | | | | | | [symbol] | [symbol] | 30804 | 11834 | 2980 | 278 | [symbol] | 0 | 0 |
| [symbol] | 148 | | | | | | | [symbol] | 371 | 22234 | 15025 | 2725 | 157 | [symbol] | 975 | 73 |
| UACC-257 | 147 | | | | | | | [symbol] | [symbol] | 72330 | 4018 | 111 | 0 | [symbol] | 0 | 311 |
| UACC-42 | 145 | | | | | | | 27441 | [symbol] | [symbol] | 202 | [symbol] | 74 | [symbol] | 346 | 271 |
| [symbol] | 144 | | | | | | | 30051 | 71 | [symbol] | 1402 | 2016 | 634 | 4067 | 175 | 274 |
| UO-31 | 143 | | | | | | | 34230 | [symbol] | [symbol] | 2316 | 644 | 1204 | 1427 | 1885 | 0 |
| [symbol] | 142 | | | | | | | [symbol] | [symbol] | 137676 | 1062 | 110 | 280 | [symbol] | 490 | 134 |
| [symbol] | 141 | | | | | | | [symbol] | 297 | [symbol] | 3025 | [symbol] | 85 | 1287 | 0 | 329 |
| [symbol] | 140 | | | | | | | 30507 | 0 | [symbol] | 2329 | 927 | 495 | [symbol] | 0 | 180 |
| MCT-15 | 139 | | | | | | | 34734 | 441 | [symbol] | 2790 | 1872 | 397 | [symbol] | 842 | 0 |
| [symbol] | 138 | | | | | | | [symbol] | [symbol] | [symbol] | 4322 | 243 | 294 | 12794 | 224 | [symbol] 1 |
| COLO [symbol] | 137 | | | | | | | 57728 | 433 | [symbol] | 2580 | 495 | 0 | [symbol] | 0 | 174 |
| LOX [symbol] | 136 | | | | | | | [symbol] | [symbol] | [symbol] | 1512 | [symbol] | 983 | [symbol] | 248 | 839 |
| [symbol] | 135 | | | | | | | [symbol] | 437 | [symbol] | 730 | [symbol] | 100 | [symbol] | 375 | [symbol] |
| [symbol] | 134 | | | | | | | 43207 | [symbol] | [symbol] | 1719 | [symbol] | 382 | 7085 | 72 | [symbol] |
| MCT 114 | 133 | | | | | | | 37406 | 345 | [symbol] | 1175 | 0 | 458 | 3813 | 721 | [symbol] |
| [symbol] | 132 | | | | | | | 34176 | [symbol] | 27523 | 0 | 534 | 757 | [symbol] | 967 | 14 |
| HCC- [symbol] | 131 | | | | | | | [symbol] | 734 | 17230 | 0 | 125 | [symbol] | [symbol] | 0 | 641 |
| [symbol] | 130 | | | | | | | [symbol] | 229 | 91730 | 1652 | 917 | 303 | [symbol] | 25 | [symbol] |
| [symbol] | 129 | | | | | | | 34742 | 33 | 90087 | 1005 | 106 | 457 | [symbol] | 819 | 0 |
| [symbol] | 128 | | | | | | | 40664 | 304 | [symbol] | 3145 | 3477 | 2041 | [symbol] | 478 | 0 |
| DU-145 | 127 | | | | | | | 37777 | [symbol] | 8182 | 5183 | 140 | 480 | [symbol] | 0 | 0 |
| [symbol] | 126 | | | | | | | 36217 | 331 | 61753 | 9431 | 94 | 827 | [symbol] | [symbol] | 110 |
| [symbol] | 125 | | | | | | | 37858 | [symbol] | [symbol] | 7367 | 1172 | 961 | [symbol] | 134 | 0 |
| [symbol] | 124 | | | | | | | 38104 | [symbol] | 106212 | 4214 | 0 | 1685 | 2875 | 423 | [symbol] |
| [symbol] | 123 | | | | | | | 23005 | 737 | 8134 | 2800 | 3182 | 625 | 13014 | 0 | 0 |
| SN12C | 122 | | | | | | | 32675 | [symbol] | 64277 | 634 | 1472 | 180 | 23636 | 0 | 637 |
| ML- [symbol] | 121 | | | | | | | 36449 | [symbol] | 2018 | 0 | 325 | 771 | 707 | [symbol] | 3 [symbol] |
| [symbol] , T-4 | 120 | | | | | | | 28305 | 783 | 17193 | 961 | 132 | 1023 | 4038 | 0 | 0 |
| OVCAR- [symbol] | 119 | | | | | | | 25375 | [symbol] | 42224 | 1995 | 2371 | 1966 | 6812 | [symbol] | 77 |
| X- [symbol] | 118 | | | | | | | 34213 | [symbol] | 18143 | 2124 | 342 | 222 | 6444 | 0 | 0 |
| OVCAR-4 | 117 | | | | | | | 42145 | 1500 | 10042 | 2320 | 2728 | 2583 | 6751 | 1276 | 1110 |
| CCRF-CEM | 116 | | | | | | | 38130 | 576 | 10022 | 2781 | 831 | 470 | 41505 | 0 | 0 |
| OVCAR-3 | 115 | | | | | | | 63017 | 548 | 47001 | 1242 | 940 | 303 | 19348 | 2443 | 0 |
| [symbol] | 114 | | | | | | | 30005 | [symbol] | 14022 | 3425 | 0 | 350 | 18283 | 0 | 252 |
| MCF- [symbol] | 113 | | | | | | | 19678 | [symbol] | 84330 | 1645 | 1196 | 406 | 1725 | 0 | 100 |
| [symbol] | 112 | | | | | | | 40230 | 257 | 17808 | 700 | 708 | 1164 | 8394 | 417 | 837 |
| [symbol] TCC | 111 | | | | | | | 47430 | 0 | 78724 | 1670 | 495 | 303 | 17019 | 1114 | 472 |
| 5F- [symbol] | 110 | | | | | | | 30345 | 64 | 64487 | 972 | 185 | 76 | 13642 | 437 | [symbol] |
| NC3- [symbol] | 109 | | | | | | | 44511 | 0 | 81076 | 585 | 813 | 276 | 4038 | 0 | 94 |
| U251 | 108 | | | | | | | 54075 | 431 | [symbol] | 573 | 134 | 713 | 17677 | 804 | 0 |
| NCL- [symbol] | 107 | | | | | | | [symbol] | [symbol] | 78179 | 436 | 405 | 603 | 17566 | 808 | 132 |
| [symbol] -75 | 106 | | | | | | | 41104 | 0 | 17012 | 403 | 416 | [symbol] | 4792 | [symbol] | 316 |
| NCI- [symbol] | 105 | | | | | | | 40037 | [symbol] | 10280 | 2064 | 4822 | 461 | 28230 | [symbol] | 0 |
| [symbol] -1 [symbol] | 104 | | | | | | | 40554 | 442 | 13825 | 1417 | 491 | 2644 | 32964 | 166 | 676 |
| NCI-H23 | 103 | | | | | | | 41587 | 633 | 6072 | 2204 | 1331 | 604 | 16328 | 743 | 60 |
| S [symbol] -OV-3 | 102 | | | | | | | 32170 | 0 | 18584 | 2641 | 522 | 200 | [symbol] | 343 | 61 |
| NCI-H23 | 101 | | | | | | | 30431 | 0 | 6416 | 4702 | 140 | 243 | [symbol] | 0 | 481 |
| [symbol] | 100 | | | | | | | 41930 | 442 | 11790 | 3091 | 229 | 303 | 11318 | 0 | 0 |
| EKVX | 99 | | | | | | | 23917 | 0 | 14173 | 1822 | 0 | 642 | 31477 | 0 | 0 |
| OVCAR-3 | 98 | | | | | | | 31129 | 0 | 7829 | 48 | 101 | 793 | 9392 | 11 | 0 |
| MCP-42 | 97 | | | | | | | 33300 | 0 | 18542 | 1074 | 384 | 465 | 12792 | 482 | 197 |
| [symbol] | 48 | | | | | | | 32440 | 0 | 9173 | 1588 | 6279 | 341 | 401 | 300 | 184 |
| [symbol] | 47 | | | | | | | 32837 | [symbol] | 0 | 2785 | 0 | 120 | 0 | 983 | 127 |
| [symbol] | 46 | | | | | | | 29124 | [symbol] | 23461 | 1117 | 0 | 92 | 10370 | 344 | 0 |
| TOGF | 24 | | | | | | | 22953 | 700 | 18106 | 7310 | [symbol] | 130 | 6277 | 1704 | 111 |
| A545-1 | | | | | | | [symbol] | 0 | 0 | 0 | 348 | 1134 | [symbol] | 415 | 571 | 0 |
| A545-3 | | | | | | | [symbol] | 0 | 0 | 0 | 871 | 134 | 4878 | 830 | 1033 | 0 |
| A545-4 | | | | | | | [symbol] | 0 | 0 | 0 | 0 | 1291 | [symbol] | 0 | [symbol] | 0 |
| A545-6 | | | | | | | [symbol] | 0 | 0 | 0 | 0 | 484 | [symbol] | 134 | 0 | 0 |
| A545-7 | | | | | | | [symbol] | 0 | 0 | 0 | 0 | 813 | [symbol] | 787 | 0 | 0 |
| EKVX-1 | | | | | | | [symbol] | 0 | 0 | 0 | 1360 | 3426 | [symbol] | 476 | 2338 | 0 |
| EKVX-4 | | | | | | | [symbol] | 0 | 0 | 0 | [symbol] | 4219 | [symbol] | 330 | 180 | 0 |
| EKVX-3 | | | | | | | [symbol] | 0 | 0 | 0 | 1806 | 0 | [symbol] | 0 | 706 | 0 |
| EKVX-6 | | | | | | | [symbol] | 0 | 0 | 0 | 284 | 1113 | [symbol] | 137 | 0 | 0 |
| EKVX-7 | | | | | | | [symbol] | 0 | 0 | 0 | 0 | 37 | 7443 | 867 | 0 | 0 |
| MCF-7-1 | | | | | | | [symbol] | 0 | 0 | 0 | 483 | 801 | 710 | 3344 | 134 | 0 |
| MCF-7-3 | | | | | | | [symbol] | 0 | 0 | 0 | 18 | 1045 | [symbol] | 0 | 482 | 0 |
| MCF-7-4 | | | | | | | [symbol] | 0 | 0 | 0 | 675 | 827 | 2087 | 218 | 13 [symbol] | 0 |
| MCF-7-5 | | | | | | | [symbol] | 0 | 0 | 0 | 1191 | 2431 | 1029 | 0 | [symbol] | 0 |
| MCF-7-7 | | | | | | | [symbol] | 0 | 0 | 0 | 0 | [symbol] | 4028 | 407 | 829 | 0 |
| ADR-RES-1 | | | | | | | [symbol] | 0 | 0 | 0 | 0 | 2734 | 433 | 4006 | [symbol] | 0 |
| ADR-RES-3 | | | | | | | [symbol] | 0 | 0 | 0 | 1145 | 3482 | 2797 | 1066 | 0 | 0 |
| ADR-RES-4 | | | | | | | [symbol] | 0 | 0 | 0 | 112 | 0 | 4038 | 637 | 801 | 0 |
| ADR-RES-5 | | | | | | | [symbol] | 0 | 0 | 0 | 403 | 2300 | 3017 | 0 | 0 | 0 |
| ADR-RES-7 | | | | | | | [symbol] | 0 | 0 | 0 | 2011 | 2904 | 842 | [symbol] | 4 [symbol] | 0 |
| WI 34-1 | | | | | | | [symbol] | 0 | 0 | 0 | 0 | 7754 | 1845 | 529 | 18311 | 0 |
| WI 34-3 | | | | | | | [symbol] | 0 | 0 | 0 | [symbol] | [symbol] | 3848 | 187 | 0 | 0 |
| WI 34-4 | | | | | | | [symbol] | 0 | 0 | 0 | 1631 | 2095 | 8250 | 0 | 1650 | 0 |
| WI 34-5 | | | | | | | [symbol] | 0 | 0 | 0 | 1300 | 7877 | 6484 | 683 | 547 | 0 |
| WI 34-7 | | | | | | | [symbol] | 0 | 0 | 0 | 1458 | 3377 | 3293 | 312 | 0 | 0 |
| [symbol] -1 | | | | | | | [symbol] E4 | 0 | 0 | 0 | [symbol] | 0 | 5828 | 0 | 80 | 0 |
| [symbol] -3 | | | | | | | [symbol] E4 | 0 | 0 | 0 | 642 | 849 | 1015 | 644 | 0 | 0 |
| [symbol] -4 | | | | | | | [symbol] E4 | 0 | 0 | 0 | 280 | 281 | 4257 | 335 | 850 | 0 |
| [symbol] -5 | | | | | | | [symbol] E4 | 0 | 0 | 0 | 1177 | 1708 | [symbol] | 1280 | 173 | 0 |
| [symbol] -7 | | | | | | | [symbol] E4 | 0 | 0 | 0 | 1543 | 274 | 4044 | 0 | 0 | 0 |
| H1 [symbol] -1 | | | | | | | [symbol] | 0 | 0 | 0 | 114 | 7085 | 4794 | 0 | 237 | 0 |
| H1 [symbol] -3 | | | | | | | [symbol] | 0 | 0 | 0 | 0 | 431 | 2347 | 542 | 906 | 0 |
| H1 [symbol] -4 | | | | | | | [symbol] | 0 | 0 | 0 | 767 | 123 | 2212 | 421 | 817 | 0 |
| H1 [symbol] -5 | | | | | | | [symbol] | 0 | 0 | 0 | 0 | 1456 | 4474 | 26 | 0 | 0 |
| H1 [symbol] -7 | | | | | | | [symbol] | 0 | 0 | 0 | 468 | 1883 | 2279 | 10 | 0 | 0 |
| [symbol] -2 | | | | | | | [symbol] | 0 | 0 | 0 | 234 | 764 | 1167 | 204 | 834 | 0 |
| EXVX-2 | | | | | | | [symbol] | 0 | 0 | 0 | 1435 | 830 | 2012 | 0 | 0 | 0 |
| HCT-114-1 | | | | | | | [symbol] | 0 | 0 | 0 | 1106 | 330 | 20773 | 1648 | 786 | 0 |
| HCT-115-2 | | | | | | | [symbol] | 0 | 0 | 0 | 2982 | 2935 | [symbol] | 148 | 811 | 0 |
| HT [symbol] -2 | | | | | | | [symbol] | 0 | 0 | 0 | 1014 | 2541 | 8264 | [symbol] | 0 | 0 |
| [symbol] -1 | | | | | | | [symbol] | 0 | 0 | 0 | 213 | 3130 | 1816 | 0 | 647 | 0 |
| [symbol] -2 | | | | | | | [symbol] | 0 | 0 | 0 | 819 | 5647 | 1019 | 793 | 179 | 0 |
| [symbol] - [symbol] -1 | | | | | | | [symbol] | 0 | 0 | 0 | 2143 | 3277 | 744 | 875 | 0 | 0 |
| [symbol] - [symbol] -2 | | | | | | | [symbol] | 0 | 0 | 0 | 326 | 5072 | 4430 | 2855 | 1204 | 0 |
| OVCAR-4-1 | | | | | | | [symbol] | 0 | 0 | 0 | 1664 | 2823 | 4847 | 25 | 867 | 0 |
| OVCAR-4-2 | | | | | | | [symbol] | 0 | 0 | 0 | 0 | 1824 | 19130 | 406 | 441 | 0 |
| OVCAR-5-1 | | | | | | | [symbol] | 0 | 0 | 0 | 1143 | 6553 | 13774 | 483 | 330 | 0 |
| OVCAR-5-2 | | | | | | | [symbol] | 0 | 0 | 0 | 1268 | 3797 | [symbol] | 875 | 0 | 0 |
| MCF-7-2 | | | | | | | [symbol] | 0 | 0 | 0 | 768 | 3082 | 1725 | 294 | 0 | 0 |
| ADA-RES-2 | | | | | | | [symbol] | 0 | 0 | 0 | 776 | 1953 | 0 | 0 | 44 | 0 |
| [symbol] -2 | | | | | | | [symbol] E4 | 0 | 0 | 0 | 841 | 1916 | 845 | 1522 | 0 | 0 |
| SW480-1 | | | | | | | [symbol] | 0 | 0 | 0 | 424 | 1870 | 4717 | [symbol] | [symbol] | 0 |
| SW480-2 | | | | | | | [symbol] | 0 | 0 | 0 | 1306 | 702 | 6413 | 0 | [symbol] | 0 |
| HT [symbol] -2 | | | | | | | [symbol] | 0 | 0 | 0 | 283 | 0 | 2907 | 1014 | 340 | 0 |
| C33A-1 | | | | | | | [symbol] | 0 | 0 | 0 | 205 | 4364 | 2016 | 131 | 440 | 0 |
| C33A-2 | | | | | | | [symbol] | 0 | 0 | 0 | 204 | 1142 | 0 | 380 | 373 | 0 |
| U [symbol] -1 | | | | | | | [symbol] | 0 | 0 | 0 | 2048 | 7970 | [symbol] | 443 | 3787 | 0 |
| U [symbol] -2 | | | | | | | [symbol] | 0 | 0 | 0 | 6100 | 11304 | 7073 | 1903 | [symbol] | 0 |
| M [symbol] -1 | | | | | | | [symbol] | 0 | 0 | 0 | 1432 | 4561 | 3320 | 0 | [symbol] | 0 |
| M [symbol] -2 | | | | | | | [symbol] | 0 | 0 | 0 | 0 | 1963 | 8724 | 283 | 0 | 0 |
| W1 [symbol] -2 | | | | | | | [symbol] | 0 | 0 | 0 | 2204 | [symbol] | 2835 | 442 | 18 | 0 |
| [symbol] -1 | | | | | | | | 0 | 0 | 0 | 1156 | 3176 | 9771 | 244 | 0 | 0 |
| [symbol] -2 | | | | | | | | 0 | 0 | 0 | 4485 | 2091 | 0 | 404 | 0 | 0 |
| [symbol] -3 | | | | | | | | 0 | 0 | 0 | 6430 | 11049 | 0 | 0 | 1371 | 0 |
| [symbol] -4 | | | | | | | | 0 | 0 | 0 | 6660 | 41955 | 13408 | 0 | 1037 | 0 |
| [symbol] -5 | | | | | | | | 0 | 0 | 0 | 4520 | 2081 | 4204 | 282 | 0 | 0 |
| [symbol] -6 | | | | | | | | 0 | 0 | 0 | 1704 | 340 | 0 | 143 | 0 | 0 |
| [symbol] -7 | | | | | | | | 0 | 0 | 0 | 3347 | 131 | 314 | 4175 | 110 | 0 |
| [symbol] -8 | | | | | | | | 0 | 0 | 0 | 48 | 21722 | 273 | 603 | 0 | 0 |
| [symbol] | | | | | | | | 0 | 0 | 0 | [symbol] | 8479 | [symbol] | 2724 | [symbol] | 0 |
| [symbol] | | | | | | | | 0 | 0 | 0 | [symbol] | [symbol] | [symbol] | [symbol] | [symbol] | 0 |
| [symbol] | | | | | | | | 0 | 0 | 0 | 8122 | 1300 | [symbol] | 1311 | [symbol] | 0 |
| [symbol] | | | | | | | | 0 | 0 | 0 | [symbol] | [symbol] | 14381 | 1851 | [symbol] | 0 |
| [symbol] | | | | | | | | 0 | 0 | 0 | 3087 | 3288 | [symbol] | 1028 | 0 | 0 |
| [symbol] | | | | | | | | 0 | 0 | 0 | [symbol] | 298 | [symbol] | 311 | 238 | 0 |
| [symbol] | | | | | | | | 0 | 0 | 0 | 3041 | 12534 | 1781 | 833 | [symbol] | 0 |
| [symbol] | | | | | | | | 0 | 0 | 0 | 4794 | 2846 | 2872 | 2303 | 4773 | 0 |
| [symbol] | | | | | | | | 0 | 0 | 0 | 1944 | 4731 | 97 | [symbol] | 933 | 0 |
| [symbol] | | | | | | | | 0 | 0 | 0 | [symbol] | [symbol] | 231 | 1117 | [symbol] | 0 |
| [symbol] | | | | | | | | 0 | 0 | 0 | 12057 | 5346 | 0 | [symbol] | [symbol] | 0 |
| [symbol] | | | | | | | [symbol] | 0 | 0 | 0 | 0 | 1444 | [symbol] | 0 | 427 | 0 |
| [symbol] | | | | | | | [symbol] | 0 | 0 | 0 | 1913 | 2753 | [symbol] | [symbol] | 17 | 0 |
| [symbol] | | | | | | | [symbol] | 0 | 0 | 0 | 742 | 2307 | 2043 | [symbol] | 819 | 0 |
| [symbol] | | | | | | | [symbol] | 0 | 0 | 0 | 614 | 8117 | 2307 | 761 | 0 | 0 |
| HC29-1 | | | | | | | [symbol] | 0 | 0 | 0 | 429 | 2044 | [symbol] | [symbol] | 0 | 0 |
| HT29-7 | | | | | | | [symbol] | 0 | 0 | 0 | 444 | 130 | 8 | 0 | 101 | 0 |
| HT29-3 | | | | | | | [symbol] | 0 | 0 | 0 | [symbol] | 2808 | 2171 | 0 | 1144 | 0 |
| [symbol] | | | | | | | [symbol] | 0 | 0 | 0 | 0 | 4143 | [symbol] | 1075 | [symbol] | 0 |
| [symbol] | | | | | | | [symbol] | 0 | 0 | 0 | [symbol] | [symbol] | 2822 | 102 | 0 | 0 |
| [symbol] | | | | | | | [symbol] | 0 | 0 | 0 | 771 | 0001 | 2712 | 1138 | 1044 | 0 |
| [symbol] | | | | | | | [symbol] | 0 | 0 | 0 | 1100 | 4034 | 0022 | 384 | 0 | 0 |
| [symbol] | | | | | | | [symbol] | 0 | 0 | 0 | 0 | 1294 | 8343 | 0 | 0 | 0 |
| [symbol] | | | | | | | [symbol] | 0 | 0 | 0 | 2050 | 2100 | 13038 | [symbol] | 3734 | 0 |
| [symbol] | | | | | | | [symbol] | 0 | 0 | 0 | 0 | 947 | 21830 | 0 | 0 | 0 |
| [symbol] | | | | | | | [symbol] | 0 | 0 | 0 | [symbol] | 2833 | 7100 | 2343 | 0 | 0 |
| [symbol] | | | | | | | [symbol] | 0 | 0 | 0 | 0 | 2474 | 6221 | 0 | 180 | 0 |
| [symbol] | | | | | | | [symbol] | 0 | 0 | 0 | [symbol] | 872 | 3321 | 0 | 0 | 0 |
| [symbol] | | | | | | | [symbol] | 0 | 0 | 0 | 1823 | 225 | 4703 | 9410 | 0 | 0 |
| [symbol] | | | | | | | [symbol] | 0 | 0 | 0 | 209 | 841 | [symbol] | 0 | 1427 | 0 |
| [symbol] | | | | | | | [symbol] | 0 | 0 | 0 | 0 | 870 | 4328 | 0 | 1188 | 0 |
| [symbol] | | | | | | | [symbol] | 0 | 0 | 0 | 1405 | 4781 | 2095 | 291 | 40 | 0 |
| [symbol] | | | | | | | [symbol] | 0 | 0 | 0 | 0 | 3029 | 2718 | 1022 | 837 | 0 |
| [symbol] | | | | | | | [symbol] | 0 | 0 | 0 | 0 | 8683 | 481 | 003 | 0 | 0 |
| [symbol] | | | | | | | [symbol] | 0 | 0 | 0 | 22351 | [symbol] | 3016 | 377 | 1215 | 0 |
| [symbol] | | | | | | | [symbol] | 0 | 0 | 0 | 31 | 4840 | 8315 | 0 | 0 | 0 |
| [symbol] | | | | | | | [symbol] | 0 | 0 | 0 | 1025 | 345 | 3156 | 144 | 0 | 0 |
| [symbol] | | | | | | | [symbol] | 0 | 0 | 0 | 1406 | 1183 | 2374 | 1002 | 0 | 0 |
| w128-6 | | | | | | | [symbol] | 0 | 0 | 0 | 1470 | 0 | 2574 | 0 | 0 | 0 |
| [symbol] | | | | | | | | 27529 | 0 | 4416 | 270 | 299 | 1447 | 6182 | 0 | 0 |
| [symbol] | | | | | | | | 25566 | 874 | 734 | 343 | 4685 | 27 | 673 | 15 | 84 |
| [symbol] | | | | | | 84 | | 34410 | 0 | 0 | 1007 | 3021 | 1562 | 0 | 228 | 0 |
| MT363 | | | | | | | | 30019 | 0 | 0 | 7007 | 21100 | 3573 | 25 | 0 | 301 |
| MT376 | | | | | | | | 37529 | 8 | 386 | 836 | 2000 | 9814 | 0 | 0 | 0 |
| MT205 | | | | | | | | 76812 | 0 | 0 | 14370 | 8054 | 1358 | 318 | 0 | 0 |
| MT206 | | | | | | | | 21786 | 831 | 0 | 0 | 32309 | 1103 | 185 | 973 | 574 |
| [symbol] | | | | | | 173 | | 38821 | 38 | 0 | 0 | [symbol] | 1010 | 75 | 175 | 0 |
| [symbol] | | | | | | 173 | | 18050 | 0 | 8 | 0 | 311 | 43 | 0 | 171 | 10 |
| [symbol] | | | | | | 177 | | 20075 | 0 | 143 | 203 | 0 | 643 | 0 | 418 | 110 |
| [symbol] | | | | | | | | 83377 | 0 | 0 | 3349 | 345 | 48 | 0 | 1700 | 0 |
| [symbol] | | | | | | 237 | | 48162 | 287 | 0 | 345 | 733 | 2244 | 400 | 105 | 0 |
| WTB-10 | | | | | | | | 40798 | 0 | 0 | 1379 | 3583 | 0 | 0 | 0 | 0 |
| [symbol] | | | | | | | | 44314 | 44 | 0 | 0 | 4810 | 70 | 0 | 261 | 0 |
| [symbol] | | | | | | | | 25725 | 7 | 8 | 0 | 8783 | 14 | 0 | 0 | 0 |
| [symbol] | | | | | | | | 24878 | 238 | 1075 | 2502 | 2701 | 1321 | 1048 | 9 | 0 |
| [symbol] | | | | | | | | 37087 | 0 | 0 | 10708 | 3881 | 674 | 0 | 0 | 0 |
| [symbol] | | | | | | | | 32134 | 0 | 281 | 17028 | 10117 | 476 | 1002 | 843 | 403 |
| MCT-115-3 | | | | | | | [symbol] | 0 | 0 | 0 | 11 | 1783 | 4484 | 207 | 24 | 0 |
| MCT-115-4 | | | | | | | [symbol] | 0 | 0 | 0 | 0 | 2318 | 7079 | 113 | 0 | 0 |
| MCT-116-6 | | | | | | | [symbol] | 0 | 0 | 0 | 1700 | 1800 | 3347 | 0 | 0 | 0 |
| MCT-116-8 | | | | | | | [symbol] | 0 | 0 | 0 | [symbol] | 2878 | 7519 | 207 | 248 | 0 |
| A545-5 | | | | | | | [symbol] | 0 | 0 | 0 | 0 | 2704 | 52 | 0 | 302 | 0 |
| MT29-3 | | | | | | | [symbol] | 0 | 0 | 0 | 148 | 4024 | 7874 | 1143 | 813 | 0 |
| [symbol] | | | | | | | [symbol] | 0 | 0 | 0 | 0 | 1848 | 2374 | 0 | 435 | 0 |
| MT29-4 | | | | | | | [symbol] | 0 | 0 | 0 | 1228 | 323 | 3223 | 181 | 0 | 0 |
| MT29-5 | | | | | | | [symbol] | 0 | 0 | 0 | 1070 | 3885 | 3007 | 0 | 383 | 0 |
| MT29-6 | | | | | | | [symbol] | 0 | 0 | 0 | 0 | 2000 | 8478 | 108 | 1056 | 0 |
| OVCAR-4-3 | | | | | | | [symbol] | 0 | 0 | 0 | 1203 | 1513 | 11854 | 079 | 800 | 0 |
| OVCAR-4-4 | | | | | | | [symbol] | 0 | 0 | 0 | 0 | 700 | 8721 | 0 | 3000 | 0 |
| OVCAR-4-5 | | | | | | | [symbol] | 0 | 0 | 0 | [symbol] | 3034 | 13754 | 1750 | 294 | 0 |
| OVCAR-4-6 | | | | | | | [symbol] | 0 | 0 | 0 | 813 | 10574 | 2375 | 211 | 786 | 0 |
| SF [symbol] -3 | | | | | | | [symbol] | 0 | 0 | 0 | 1203 | 4100 | 1572 | 158 | 108 | 0 |
| SF [symbol] -4 | | | | | | | [symbol] | 0 | 0 | 0 | 0 | 1003 | 3754 | 0 | 0 | 0 |
| SF [symbol] -5 | | | | | | | [symbol] | 0 | 0 | 0 | [symbol] | 2893 | 2303 | 414 | 504 | 0 |
| SF [symbol] -6 | | | | | | | [symbol] | 0 | 0 | 0 | [symbol] | 4180 | 0 | 540 | 111 | 0 |
| OVCAR-3 | | | | | | | [symbol] | 0 | 0 | 0 | 1547 | 6437 | 7120 | 0 | 400 | 0 |
| OVCAR-4 | | | | | | | [symbol] | 0 | 0 | 0 | 0 | 3700 | 3410 | 28 | 0 | 0 |
| OVCAR-5 | | | | | | | [symbol] | 0 | 0 | 0 | 234 | 208 | 1823 | 13 | 0 | 0 |
| OVCAR-6 | | | | | | | [symbol] | 0 | 0 | 0 | 634 | [symbol] | 2247 | 433 | 718 | 0 |
| ADR- [symbol] | | | | | | | [symbol] | 0 | 0 | 0 | 0 | 2828 | 2854 | 94 | 162 | 0 |
| MCF-7-6 | | | | | | | [symbol] | 0 | 0 | 0 | 1327 | 1664 | 4791 | 128 | 0 | 0 |
| M [symbol] | | | | | | | [symbol] | 0 | 0 | 0 | [symbol] | 1994 | 372 | 0 | [symbol] | 0 |
| SW200-5 | | | | | | | [symbol] | 0 | 0 | 0 | 0 | 0 | 2309 | 283 | 0 | 0 |
| SW400-3 | | | | | | | [symbol] | 0 | 0 | 0 | 0 | 058 | 3879 | 303 | 2519 | 0 |
| SW400-4 | | | | | | | [symbol] | 0 | 0 | 0 | [symbol] | 377 | 4245 | 300 | 1054 | 0 |
| SW400-5 | | | | | | | [symbol] | 0 | 0 | 0 | 2315 | 1275 | 4525 | 10 | 14325 | 0 |
| SW400- [symbol] | | | | | | | [symbol] | 0 | 0 | 0 | 1702 | 1026 | 1983 | [symbol] | 1577 | 0 |
| C33A-2 | | | | | | | [symbol] | 0 | 0 | 0 | 0 | 0 | 451 | 0 | 294 | 0 |
| C33A-4 | | | | | | | [symbol] | 0 | 0 | 0 | 72 | 775 | 0 | 9 | 808 | 0 |
| C33A-6 | | | | | | | [symbol] | 0 | 0 | 0 | 0 | 5457 | 21373 | 9 | 0 | 0 |
| C33A-4 | | | | | | | [symbol] | 0 | 0 | 0 | 0 | 10 | 8473 | 0 | 0 | 0 |
| [symbol] | | | | | | | [symbol] | 0 | 0 | 0 | 573 | 41 | 8821 | 2851 | 764 | 0 |
| [symbol] -3 | | | | | | | [symbol] | 0 | 0 | 0 | 1430 | 11023 | [symbol] | 10043 | 1183 | 0 |
| [symbol] -4 | | | | | | | [symbol] | 0 | 0 | 0 | 1190 | 8148 | [symbol] | 1918 | 2063 | 0 |
| [symbol] -5 | | | | | | | [symbol] | 0 | 0 | 0 | 1754 | 6154 | 2500 | 0 | 2300 | 0 |
| [symbol] -6 | | | | | | | [symbol] | 0 | 0 | 0 | [symbol] | 17007 | 318 | [symbol] | 219 | 0 |
| [symbol] -4 | | | | | | | [symbol] | 0 | 0 | 0 | 1629 | 2315 | 2548 | [symbol] | 0 | 0 |
| [symbol] -3 | | | | | | | [symbol] | 0 | 0 | 0 | [symbol] | 0007 | 7181 | 105 | 278 | 0 |
| [symbol] -4 | | | | | | | [symbol] | 0 | 0 | 0 | 27 [symbol] | 1182 | 20533 | 1017 | 0 | 0 |
| SF- [symbol] | | | | | | | [symbol] | 0 | 0 | 0 | 1124 | 6180 | 6431 | 0 | 0 | 0 |
| SF-299-4 | | | | | | | [symbol] | 0 | 0 | 0 | 1417 | 2982 | 6331 | 1674 | 22 | 0 |
| SF- [symbol] | | | | | | | [symbol] | 0 | 0 | 0 | 1834 | 3020 | 3241 | 0 | 9 | 0 |
| SF- [symbol] | | | | | | | [symbol] | 0 | 0 | 0 | 1514 | 4288 | 84 | 402 | 0 | 0 |
| [symbol] -13 | | | | | | | | 0 | 0 | 0 | 5018 | 12523 | 12085 | [symbol] | 0005 | 0 |
| [symbol] -20 | | | | | | | | 0 | 0 | 0 | 4071 | 10817 | 13500 | [symbol] 47 | 7180 | 0 |
| [symbol] -21 | | | | | | | | 0 | 0 | 0 | 2612 | 4067 | 7000 | [symbol] | 2787 | 0 |
| [symbol] -22 | | | | | | | | 0 | 0 | 0 | [symbol] | 3314 | 2833 | 7314 | 681 | 0 |
| OVCAR-5 | | | | | | | [symbol] | 0 | 0 | 0 | 4 [symbol] | 8671 | 4413 | 200 | 2240 | 0 |
| [symbol] -10 | | | | | | | | 0 | 0 | 0 | 3474 | 838 | 478 | 143 | 4303 | 0 |
| [symbol] -11 | | | | | | | | 0 | 0 | 0 | 2220 | 0 | 12 | 205 | 4983 | 0 |
| [symbol] -12 | | | | | | | | 0 | 0 | 0 | 1477 | 2702 | 1025 | 812 | 17000 | 0 |
| [symbol] -13 | | | | | | | | 0 | 0 | 0 | 1770 | 11411 | 1101 | 2922 | 251 | 0 |
| [symbol] -14 | | | | | | | | 0 | 0 | 0 | 3018 | 0 | 0 | 3030 | 61 | 0 |
| [symbol] -15 | | | | | | | | 0 | 0 | 0 | 1200 | 13215 | 645 | 747 | 4236 | 0 |
| [symbol] -16 | | | | | | | | 0 | 0 | 0 | 1737 | 902 | 4334 | 420 | [symbol] | 0 |
| [symbol] -17 | | | | | | | | 0 | 0 | 0 | 2243 | 14 | 0 | 573 | 433 | 0 |
| [symbol] -18 | | | | | | | | 0 | 0 | 0 | 3478 | [symbol] | 4281 | 1120 | 502 | 0 |
| [symbol] -19 | | | | | | | | 0 | 0 | 0 | 1934 | 12800 | 4418 | 653 | 1028 | 0 |
| [symbol] | | 1 | | | | | | 18824 | 0 | 236 | 184 | 1815 | 2243 | 1782 | [symbol] | [symbol] |
| [symbol] | | 2 | | | | | | 2314 | 0 | 262 | 384 | 2374 | 1794 | [symbol] | [symbol] | [symbol] |
| [symbol] | | 3 | | | | | | 1 [symbol] | 0 | 237 | 0 | [symbol] | [symbol] | 1381 | 1112 | 42217 |
| [symbol] | | 4 | | | | | | 441 | 0 | 0 | 0 | 2706 | 1043 | [symbol] | 0 | 2239 |
| [symbol] | | 5 | | | | | | 4 [symbol] | 0 | 100 | 234 | 1426 | [symbol] | [symbol] | 11234 | 31129 |
| [symbol] | | 6 | | | | | | 3 [symbol] | 0 | 0 | 344 | [symbol] 76 | 3342 | [symbol] | 8722 | 44330 |
| [symbol] | | 7 | | | | | | [symbol] | 0 | 30 | 0 | [symbol] | [symbol] | 3100 | 13783 | [symbol] |
| [symbol] | | 8 | | | | | | 15879 | 0 | 82 | 231 | 4629 | 8387 | 232 | 32283 | [symbol] |
| [symbol] | | 9 | | | | | | 52149 | 0 | 253 | [symbol] | 1743 | [symbol] | 2001 | [symbol] | [symbol] |
| [symbol] | | 10 | | | | | | 4434 | 0 | 51 | 87 | 1183 | 2209 | 2312 | 41000 | 33000 |
| [symbol] | | 11 | | | | | | 2337 | 0 | 223 | 0 | 1334 | [symbol] | 1334 | 30344 | [symbol] |
| [symbol] | | 12 | | | | | | 2083 | 0 | 114 | 0 | [symbol] | 3783 | 1334 | [symbol] | 21152 |
| [symbol] | | 13 | | | | | | 2878 | 0 | 103 | 100 | [symbol] | [symbol] | 334 | 4384 | 42100 |
| [symbol] | | 14 | | | | | | 0 | 0 | 138 | 0 | [symbol] | 3419 | 119 | 1431 | 20023 |
| [symbol] | | 15 | | | | | | [symbol] | 0 | 218 | 348 | 10713 | 4379 | 1129 | [symbol] | [symbol] |
| [symbol] | | 16 | | | | | | [symbol] | 0 | 0 | 0 | [symbol] | 1942 | 1183 | 2004 | 14412 |
| [symbol] | | 17 | | | | | | [symbol] 42 | 0 | [symbol] | 181 | 1123 | 472 | 1022 | 2810 | 14747 |
| [symbol] | | 18 | | | | | | 37 [symbol] | 0 | [symbol] | 144 | 2037 | [symbol] | 443 | 4172 | 22300 |
| [symbol] | | 19 | | | | | | 0 | 0 | 181 | 223 | [symbol] | [symbol] | 431 | 70 [symbol] | [symbol] |
| [symbol] | | 20 | | | | | | 0 | 0 | 134 | 0 | [symbol] | [symbol] | 272 | [symbol] | [symbol] |
| [symbol] | | 21 | | | | | | 8348 | 0 | 0 | 0 | [symbol] | 0 | 281 | 0 | [symbol] |
| [symbol] | | 22 | | | | | | 2383 | 0 | 0 | 184 | 1447 | 3300 | 831 | [symbol] | [symbol] |
| [symbol] | | 23 | | | | | | 2011 | 0 | 104 | 177 | [symbol] | 2134 | 473 | 3434 | 20017 |
| [symbol] | | 24 | | | | | | [symbol] 2 | 0 | 125 | 0 | [symbol] | 412 | 231 | 1243 | 17007 |
| [symbol] | | 25 | | | | | | [symbol] 45 | 0 | 1100 | 200 | [symbol] | [symbol] | 11004 | [symbol] | 48517 |
| [symbol] | | 27 | | | | | | 1202 | 0 | 47 | 23 | [symbol] | [symbol] | 1430 | 11400 | 85005 |
| [symbol] | | 28 | | | | 28 | | 384 | 0 | 0 | [symbol] | [symbol] | 0 | 0 | 0 | 1074 |
| [symbol] | | 29 | | | | | | [symbol] 25 | 0 | 134 | 0 | 1324 | 1000 | 234 | 3454 | 21000 |
| [symbol] | | 30 | | | | 30 | | 100 | 0 | 11 | [symbol] | 4134 | [symbol] | 84 | 0 | [symbol] |
| [symbol] | | 31 | | | | | | 3411 | 0 | 17 | 0 | 4408 | 4182 | 1387 | [symbol] | [symbol] |
| [symbol] | | 32 | | | | | | 0 | 0 | 33 | [symbol] | 1237 | 144 | 19 | 383 | [symbol] |
| [symbol] | | 33 | | | | | | 3091 | 0 | 42 | 307 | 1237 | 1343 | 1200 | 1300 | 38300 |
| [symbol] | | 34 | | | | | | 194 | 0 | 20 | 70 | 1731 | [symbol] | 278 | [symbol] | [symbol] |
| [symbol] | | 35 | | | | | | 179 | 0 | 0 | 0 | 1300 | 0 | 0 | 1 | 21419 |
| [symbol] | | 36 | | | | | | [symbol] | 0 | 114 | 0 | 1323 | [symbol] | 182 | [symbol] | [symbol] |
| [symbol] | | 37 | | | | | | [symbol] | 0 | 14 | 0 | 2413 | 0 | 31 | 0 | 2343 |
| [symbol] | | 38 | | | | | | [symbol] 223 | 0 | 8 | 0 | [symbol] | 1072 | 134 | 0 | [symbol] |
| [symbol] | | 39 | | | | | | [symbol] | 0 | 17 | 33 | [symbol] | [symbol] | 0 | 0 | 2701 |
| [symbol] | | 40 | | | | | | 100 | 0 | 0 | [symbol] | 4023 | [symbol] | 0 | 0 | [symbol] |
| [symbol] | | 41 | | | | | | 92 | 0 | 0 | 0 | 2089 | 0 | 0 | 0 | 2873 |
| [symbol] | | 42 | | | | | | 11823 | 0 | 0 | 0 | [symbol] | [symbol] | 142 | 0 | [symbol] |
| [symbol] | | 43 | | | | | | 0 | 0 | 0 | 407 | 1511 | 1427 | 0 | 0 | [symbol] |
| [symbol] | | 44 | | | | | | 1132 | 0 | 27 | 83 | [symbol] | [symbol] | 118 | [symbol] | 22783 |
| [symbol] | | | | | 343 | | | 871 | 0 | 0 | 0 | [symbol] | 0 | 0 | 0 | 0 |
| [symbol] | | | | | 343 | | | 0 | 0 | 0 | 0 | 295 | 0 | 32 | 0 | 482 |
| [symbol] | | | | | 351 | | | 2134 | 0 | 0 | 47 | 1307 | 387 | 21 | 0 | [symbol] |
| [symbol] | | | | | 3 [symbol] | 356 | | 1100 | 0 | 0 | 403 | 1430 | 2007 | [symbol] | 0 | [symbol] |
| [symbol] | | | | | 334 | 334 | | [symbol] 27 | 0 | 49 | 0 | 1405 | 384 | [symbol] | 334 | 2334 |
| [symbol] | | | | | 344 | | | 0 | 0 | 11 | 0 | 1862 | 487 | 00 | 200 | 1728 |
| [symbol] | | | | | 342 | | | 0 | 0 | 0 | 0 | 1829 | 1170 | 0 | 234 | 7342 |
| [symbol] | | | | | 334 | 334 | | 0 | 0 | 0 | 209 | [symbol] | 117 | 0 | 0 | 0 |
| [symbol] | | | | | 332 | | | 2714 | 0 | 0 | 0 | 4034 | 0 | 421 | 0 | 27819 |
| [symbol] | | | | | 3 [symbol] | | | 0 | 0 | 0 | 324 | 4482 | 0 | [symbol] | 0 | 191 |
| [symbol] | | | | | 334 | | | 3524 | 0 | 200 | 10 | [symbol] | 20 | [symbol] | 4380 | 13417 |
| [symbol] | | | | | 327 | | | 0 | 0 | 114 | 378 | 4437 | 0 | 0 | 42 | 400 |
| [symbol] | | | | | 3 [symbol] | | | 2912 | 0 | 0 | 230 | 1683 | 0 | 423 | 347 | [symbol] |
| [symbol] | | | | | 321 | | | 240 | 0 | 0 | 0 | 3430 | 0 | 0 | 0 | 1102 |
| [symbol] | | | | | 320 | | | 1442 | 0 | 0 | 0 | 3421 | 0 | 0 | 0 | 3447 |
| [symbol] | | | | | 318 | | | 3528 | 0 | 313 | 0 | [symbol] | 4543 | 0 | 0 | [symbol] |
| [symbol] | | | | | 316 | | | 1424 | 0 | 20 | 184 | 4057 | 2227 | 187 | [symbol] | [symbol] |
| [symbol] | | | | | 314 | | | 3099 | 0 | 220 | 175 | 1272 | 443 | 343 | [symbol] | [symbol] |
| [symbol] | | | | | 311 | | | 0 | 0 | 84 | 0 | [symbol] | 410 | 27 | 0 | 17721 |
| [symbol] | | | | | 309 | | | 230 | 0 | 0 | 0 | 1414 | 700 | 0 | 0 | [symbol] |
| [symbol] | | | | | 307 | | | 0 | 0 | 0 | 370 | 3424 | 0 | [symbol] | 1841 | [symbol] |
| [symbol] | | | | | 305 | | | 0 | 0 | 34 | 260 | 4133 | 2282 | [symbol] | 1000 | [symbol] |
| [symbol] | | | | | 303 | | | 3 [symbol] | 0 | 382 | 53 | 8370 | 4071 | 04 | 0 | 37185 |
| [symbol] | | | | | [symbol] | | | [symbol] | 0 | 376 | 164 | [symbol] | [symbol] | 145 | [symbol] | [symbol] |
| [symbol] | | | | | 2 [symbol] | | | [symbol] 378 | 0 | 233 | 64 | [symbol] | [symbol] | [symbol] | 2087 | [symbol] |
| [symbol] | | | | | 207 | | | 3054 | 0 | 0 | 434 | 4472 | 0 | 44 | 1583 | 30382 |
| [symbol] | | | | | 2 [symbol] | | | 248 | 0 | 134 | 0 | 3416 | 3028 | 0 | 482 | [symbol] |
| [symbol] | | | | | 234 | | | 0 | 0 | [symbol] | 0 | [symbol] | 2048 | 304 | 543 | 20075 |
| [symbol] | | | | | 242 | | | 221 | 0 | [symbol] | 0 | 2032 | [symbol] | 178 | 134 | 18371 |
| [symbol] | | | | | 2 [symbol] | | | [symbol] 03 | 0 | 245 | 0 | 12 | [symbol] | 54 | 124 | 40024 |
| [symbol] | | | | | 279 | | | 0 | 0 | [symbol] | 234 | 1440 | 114 | 0 | 303 | 1531 |
| [symbol] | | | | | 277 | | | 100 | 0 | 0 | 0 | 4734 | 1182 | 0 | 1304 | 10122 |
| [symbol] | | | | | 275 | 275 | | 0 | 0 | [symbol] 1 | 181 | 3704 | 474 | 83 | 0 | 0 |
| [symbol] | | | | | 2 [symbol] | | | 0 | 0 | 7 | 0 | [symbol] | 242 | 434 | 0 | 6225 |
| [symbol] | | | | | 2 [symbol] | | | 1297 | 0 | 0 | 116 | [symbol] | 383 | 84 | 474 | 11174 |
| [symbol] | | | | | 236 | 236 | | 0 | 0 | 100 | 0 | [symbol] | 7082 | 0 | 1824 | 13782 |
| [symbol] | | | | | 235 | 235 | | 83 | 0 | 134 | 0 | [symbol] | [symbol] | 0 | 52 | 4342 |
| [symbol] | | | | | 234 | | | 0 | 0 | 0 | 0 | [symbol] | 142 | 160 | 0 | 2417 |
| [symbol] | | | | | 233 | 233 | | [symbol] 71 | 0 | 47 | 0 | [symbol] | 0 | 0 | 0 | 1131 |
| [symbol] | | | | | 231 | 231 | | 0 | 0 | 130 | 100 | 4134 | 2231 | 110 | 417 | 1492 |
| [symbol] | | | | | 229 | 229 | | 0 | 0 | [symbol] | 100 | 3310 | 4000 | 112 | 0 | 1100 |
| [symbol] | | | | | 227 | 227 | | 0 | 0 | 187 | 8 | 4156 | 117 | 0 | 0 | 4183 |
| [symbol] | | | | | 222 | | | 0 | 0 | 14 | 3 | 5439 | 0 | 83 | 0 | 1054 |
| [symbol] | | | | | 218 | | | 18777 | 0 | 0 | 124 | 2347 | 0 | 0 | 434 | [symbol] |
| [symbol] | | | | | 214 | | | 1984 | 0 | 41 | 143 | [symbol] | 1000 | 0 | 138 | 33000 |
| [symbol] | | | | | 213 | | | 0 | 0 | [symbol] | 70 | 3333 | 0 | 132 | 0 | [symbol] |
| [symbol] | | | | | 212 | | | [symbol] | 0 | 0 | 77 | 1011 | 1004 | 134 | 17 | 18058 |
| [symbol] | | | | | 211 | 211 | | 107 | 0 | 0 | 0 | 1206 | 241 | 0 | 0 | 149 |
| [symbol] | | | | | 210 | | | [symbol] 2 | 0 | 113 | 0 | 1400 | 4313 | 100 | 001 | [symbol] |
| [symbol] | | | | | 209 | | | 30 | 0 | 11 | 0 | 2424 | 3335 | 34 | 104 | 316 |
| [symbol] | | | | | 289 | | | [symbol] | 0 | 0 | 172 | 4207 | 0 | 0 | 0 | 3834 |
| [symbol] | | | | | [symbol] | | | 1319 | 0 | 0 | 0 | 2439 | 0 | 110 | 0 | 270 |
| [symbol] | | | | | 201 | | | 182 | 0 | 29 | 0 | 2170 | [symbol] | 0 | 0 | 4131 |
| [symbol] | | | | | 1 [symbol] | | | 204 | 0 | 11 | 0 | [symbol] | 0 | 20 | 0 | [symbol] |
| [symbol] | | | | | 137 | | | 180 | 0 | 11 | 0 | 2207 | [symbol] | 12 | 4200 | [symbol] |
| [symbol] | | | | | 186 | | | 0 | 0 | [symbol] 4 | 0 | 1347 | 174 | 42 | 23 | 1813 |
| [symbol] | | | | | 183 | | | 110 | 0 | 13 | 0 | 2304 | [symbol] | 11 | 0 | 20071 |
| [symbol] | | | | | 178 | | | 0 | 0 | 0 | 0 | 1139 | 0 | 0 | 400 | 10 |
| [symbol] | | | | | 81 | | | 0 | 0 | 0 | [symbol] | [symbol] | [symbol] | 0 | 0 | [symbol] |
| [symbol] | | | | | [symbol] | | | 1428 | 0 | 0 | 0 | [symbol] | 1000 | 0 | 0 | [symbol] |
| [symbol] | | | | | [symbol] 7 | | | [symbol] | 0 | 0 | 0 | 4477 | 2000 | 24 | 384 | 23334 |
| [symbol] | | | | | [symbol] 4 | | | 242 | 0 | [symbol] 4 | 0 | [symbol] | 241 | 70 | 0 | 8333 |
| [symbol] | | | | | [symbol] 3 | | | 0 | 0 | 0 | 0 | 1711 | 142 | 0 | 137 | 410 |
| [symbol] | | | | | [symbol] 1 | | | 174 | 0 | 23 | 242 | 2021 | 2134 | 0 | 187 | [symbol] |
| [symbol] | | | | | [symbol] | | | 0 | 0 | [symbol] | 0 | 3843 | 824 | 0 | 81 | 12023 |
| [symbol] | | | | 79 | | | | 0 | 0 | 0 | 70 | 4670 | 341 | 0 | 0 | 37319 |
| [symbol] | | | | 81 | | | | 0 | 0 | 42 | 2700 | 4053 | 237 | 0 | 31 | 3014 |
| [symbol] | | | | 83 | | | | 143 | 0 | [symbol] | 0 | [symbol] | 4007 | 0 | 288 | 1371 |
| [symbol] | | | | [symbol] | | | | [symbol] | 0 | 30 | 0 | [symbol] | 0 | 0 | 131 | [symbol] |
| [symbol] | | | | [symbol] | | | | 200 | 0 | 0 | 343 | 7318 | 425 | 0 | 0 | [symbol] |
| [symbol] | | | | 90 | | | | 0 | 0 | 183 | 0 | 1780 | 701 | 443 | 0 | [symbol] |
| [symbol] | | | | 82 | | | | 53 | 0 | 70 | 12 | [symbol] 2 | 731 | 43 | 0 | [symbol] |
| [symbol] | | | | 94 | | | | 483 | 0 | 0 | 0 | [symbol] 57 | 0 | 230 | 0 | 1053 |
| [symbol] | | | | [symbol] | | | | [symbol] | 0 | [symbol] | 0 | 4334 | 4121 | 12 | 0 | 1789 |
| [symbol] | | | | 14 [symbol] | | | | [symbol] | 0 | 0 | 0 | 2707 | [symbol] | 0 | [symbol] | [symbol] |
| [symbol] | | | | 14 [symbol] | | | | 22 | 0 | 0 | 183 | 27 [symbol] | 80 | 143 | 1739 | 425 |
| [symbol] | | | | 150 | | | | 0 | 0 | 30 | 0 | 1047 | 1083 | 184 | 730 | 2441 |
| [symbol] | | | | 152 | | | | 2072 | 0 | 0 | 247 | 2284 | 0 | 343 | 3370 | 2024 |
| [symbol] | | | | 154 | | | | [symbol] | 0 | 0 | 0 | 2623 | 1100 | 0 | [symbol] | 3807 |
| [symbol] | | | | 1 [symbol] | | | | 0 | 0 | 42 | 207 | 1432 | 0 | 0 | 114 | 1632 |
| [symbol] | | | | 154 | | | | 0 | 0 | 42 | 570 | 184 | 0 | [symbol] | 1114 | 0 |
| [symbol] | | | | 109 | | | | 0 | 0 | 52 | 149 | 1729 | 1178 | 0 | 0 | 231 |
| [symbol] -143 | | | | 142 | | | | 0 | 0 | 43 | 0 | 1422 | 217 | [symbol] | 1034 | 1800 |
| [symbol] | | | | 184 | | | | 12284 | 0 | 0 | 0 | 1344 | 0 | 183 | 1004 | 4034 |
| C. [symbol] | | | | [symbol] | | | | [symbol] | 0 | 47 | [symbol] | [symbol] 12 | [symbol] | 784 | [symbol] | [symbol] |
| [symbol] | | | | [symbol] | | | | 4017 | 0 | 0 | [symbol] | [symbol] | 04 | 0 | 21342 | [symbol] |
| T-4 [symbol] | | | | 100 | | | | 0 | 0 | 70 | 212 | [symbol] | 73 | [symbol] | [symbol] 531 | 7187 |
| [symbol] 3 | | | | 171 | | | | 0 | 0 | 0 | [symbol] | [symbol] | 373 | 0 | 0 | 241 |
| c [symbol] , 1441 [symbol] 30 | | | | 181 | | | | 0 | 0 | 0 | [symbol] | 13 [symbol] | 130 | 203 | [symbol] | 0 |
| 7817 [symbol] * c [symbol] | | | | 143 | | | | 0 | 0 | 31 | 0 | 447 | 0 | 1 | 212 | 410 |
| KB [symbol] A* | | | | 1 [symbol] 4 | | | | 0 | 0 | 0 | 135 | [symbol] | 24 | 52 | 211 | [symbol] |
| [symbol] poly A* | | | | [symbol] | | | | 282 | 0 | 43 | 0 | 2437 | 0 | [symbol] | [symbol] | 3781 |
| ACI-94 | | | | 124 | | | | 0 | 0 | 0 | 0 | 2832 | 208 | 171 | 8742 | [symbol] |
| UACC-02 | | | | 209 | | | | 304 | 0 | 34 | 131 | 1204 | 0 | 0 | 1277 | 1373 |
| MCF-7 [symbol] | | | | 202 | | | | 0 | 0 | 34 | 0 | 1347 | 700 | 0 | 1434 | 1030 |
| UTOS ( [symbol] | | | | 204 | | | | 0 | 0 | 171 | 22 | 0 | 0 | 0 | 0 | 3433 |
| WISM (C [symbol] A* | | | | 206 | | | | 0 | 0 | 0 | 0 | 742 | 843 | 44 | 42 | 1191 |
| 454 [symbol] | | | | 208 | | | | [symbol] 41 | 0 | 0 | 0 | 7137 | 0 | 0 | 0 | [symbol] |
| CC1, 137 [symbol] | | | | 218 | | | | 0 | 0 | 80 | 141 | 1184 | 3 | 100 | 0 | 8.27 |
| W [symbol] 10% PBS | | | | 219 | | | | 0 | 0 | 0 | 0 | 2120 | 0 | 77 | 0 | [symbol] |
| CR [symbol] 1441 * TFA [symbol] | | | | 220 | | | | 0 | 0 | 0 | 234 | [symbol] | 03 | 0 | 0 | 0 |
| [symbol] -1 | | | | 221 | | | | [symbol] | 0 | 0 | 16 | 1672 | 0 | 0 | 0 | 1347 |
| [symbol] -2 | | | | 223 | | | | 0 | 0 | 0 | 0 | 3225 | 300 | [symbol] | 0 | [symbol] |
| K [symbol] -4 | | | | 224 | | | | 734 | 0 | 172 | 0 | [symbol] | 0 | 0 | 0 | 1331 |
| MCP-02 | | | | 241 | | | | 124 | 0 | 0 | 3 | 1134 | 372 | 0 | 0 | 1724 |
| [symbol] , T-4 | | | | 242 | | | | 1229 | 0 | 0 | 0 | 41 [symbol] | 1234 | 0 | 2 [symbol] 52 | [symbol] |
| DXYX | | | | 243 | | | | [symbol] | 0 | 0 | [symbol] | 2700 | [symbol] | 317 | [symbol] 10 | 1440 |
| L-40 | | | | 244 | | | | 0 | 0 | 0 | 0 | 3432 | [symbol] | 614 | 14 [symbol] | 10317 |
| MC-4423 | | | | 245 | | | | 237 | 0 | [symbol] | 183 | [symbol] | [symbol] | 321 | 3342 | 6330 |
| [symbol] 8226 | | | | [symbol] | | | | 0 | 0 | [symbol] | 413 | 2047 | 711 [symbol] | 182 | 0 | 736 |
| AS [symbol] A TCC | | | | 247 | | | | 0 | 0 | [symbol] | 0 | 2108 | 1 [symbol] 10 | 33 | 241 | [symbol] |
| SA | | | | 248 | | | | 0 | 0 | 0 | 0 | 2709 | 1310 | 23 | 261 | [symbol] |
| CVCAR-3 | | | | 249 | | | | 0 | 0 | 17 | 247 | 3481 | 0 | 41 | 183 | [symbol] 44 |
| MCT-14 | | | | 250 | | | | 1042 | 0 | 0 | 62 | [symbol] | 1387 | 283 | [symbol] | [symbol] |
| OVC [symbol] -4 | | | | 251 | | | | 83 | 0 | 83 | 1 [symbol] | 542 | 0 | 7 | [symbol] | [symbol] 7 |
| UO-31 | | | | 252 | | | | 347 | 0 | 0 | [symbol] | [symbol] | 0 | 0 | [symbol] | 1047 |
| OVCAR-5 | | | | 253 | | | | 0 | 0 | [symbol] | 727 | [symbol] | 0 | 362 | 4789 | [symbol] 32 |
| SM12C | | | | 254 | | | | 811 | 0 | 0 | 0 | 355 [symbol] | [symbol] | 0 | [symbol] | [symbol] |
| OVCAR-4 | | | | 256 | | | | 322 | 0 | 0 | 13 | [symbol] | 872 | 0 | [symbol] | 1742 |
| LOX [symbol] | | | | 25 [symbol] | | | | 0 | 0 | 77 | 343 | 8301 | [symbol] | [symbol] | 2950 | 8100 |
| X3 [symbol] OV1 | | | | 257 | | | | [symbol] | 0 | [symbol] | 0 | [symbol] | [symbol] | 2 | [symbol] | [symbol] 37 |
| SX [symbol] -2 | | | | 258 | | | | 0 | 0 | 176 | 0 | 23 [symbol] | 401 | 338 | 1574 | [symbol] 04 |
| SX-OV-3 | | | | 259 | | | | 0 | 0 | 0 | 0 | 1164 | 0 | 184 | 14 [symbol] | 1437 |
| SX-MEL-4 | | | | 260 | | | | 0 | 0 | 0 | 472 | 1378 | [symbol] | 0 | [symbol] | 14 [symbol] |
| SF- [symbol] | | | | 261 | | | | 1373 | 0 | [symbol] | 434 | [symbol] 73 | 3291 | 0 | 7434 | [symbol] |
| [symbol] | | | | 262 | | | | [symbol] 67 | 0 | 0 | 0 | 3780 | [symbol] 41 | 0 | [symbol] | 3063 |
| K-962 | | | | 263 | | | | 157 | 0 | 85 | 31 | 8144 | 4336 | 276 | 2537 | 1782 |
| UACC-287 | | | | 264 | | | | [symbol] | 0 | 0 | 0 | [symbol] | 0 | 42 | 0 | 1200 |
| M14 | | | | 265 | | | | 0 | 0 | 74 | 0 | [symbol] 27 | 151 | 181 | 0 | 1430 |
| MCF7 | | | | 267 | | | | 5820 | 0 | 0 | 24 | 8234 | 0 | 0 | 17404 | [symbol] |
| MOA-M-436 | | | | 269 | | | | 0 | 0 | 80 | 227 | 1834 | 0 | 0 | 744 | 1343 |
| MT279 | | | | 270 | | | | 1743 | 0 | 0 | 0 | 1534 | 2 [symbol] 3 | 87 | 0 | [symbol] |
| MOA-N | | | | 271 | | | | 0 | 0 | 17 | 0 | 4727 | [symbol] | 283 | 1282 | 3025 |
| v10 poly A* | | | | 273 | | | | [symbol] 006 | 0 | 137 | 0 | 7232 | 1800 | 43 | 854 | 18372 |
| XHOS poly A* | | | | [symbol] | | | | 370 [symbol] | 0 | 14 | [symbol] | 3301 | 32 | 384 | 579 | 4284 |
| HT2.34 24th TPA RHA [symbol] 23 | | | | 300 | | | | 0 | 0 | 19 | 0 | 2942 | [symbol] | 171 | [symbol] 4 | [symbol] |
| HELA-EXP-031000 | | | | 313 | | | | 0 | 0 | 19 | 49 | 2177 | 949 | 24 | 0 | 104 [symbol] |
| HT384 [symbol] | | | | 322 | | | | 630 | 0 | 0 | [symbol] | 4154 | [symbol] | 424 | 773 | 4214 |
| HT347 | | | | 323 | | | | 442 | 0 | 5 | 0 | 244 [symbol] | 2412 | 0 | [symbol] | 54352 |
| 456 [symbol] | | | | 324 | | | | [symbol] | 0 | 55 | 0 | 3464 | 0 | 11 | 761 | 1101 |
| NCI- [symbol] 224 | | | | 334 | | | | 0 | 0 | 0 | 34 | [symbol] | 0 | 100 | 0 | 1100 |
| [symbol] | | | | 337 | | | | 0 | 0 | 0 | 357 | 3132 | 381 | 181 | [symbol] 222 | [symbol] 479 |
| [symbol] -231 | | | | 338 | | | | 7140 | 0 | 22 | 0 | 8113 | 4366 | 0 | [symbol] | 28631 |
| U251 | | | | 339 | | | | 16344 | 0 | [symbol] | 0 | 116 [symbol] | 18310 | 202 | 141005 | 34705 |
| FT [symbol] poly A* | | | | 340 | | | | 0 | 0 | 0 | 0 | [symbol] | 0 | 0 | [symbol] | 2606 |
| FC-3 | | | | 341 | | | | 0 | 0 | 0 | 227 | 3879 | [symbol] | 63 | 584 | 419 |
| HCC-2 [symbol] | | | | 343 | | | | 31 | 0 | 0 | 0 | 2399 | 0 | 138 | 0 | [symbol] |
| SW-420 | | | | 345 | | | | 0 | 0 | 72 | 83 | 2872 | 0 | 106 | [symbol] | 0 |
| MT102 | | | | 346 | | | | 575 | 0 | 80 | 25 | 4309 | [symbol] | 0 | 3475 | 10731 |
| COLO 204 | | | | 347 | | | | 0 | 0 | 24 | 43 | 703 | [symbol] | 0 | 0 | 0 |
| MT218 | | | | 348 | | | | 4211 | 0 | 0 | 0 | 1703 | 0 | 0 | 0 | 1866 |
| [symbol] -12 | | | | 349 | | | | 0 | 0 | 44 | 137 | 1734 | 842 | 102 | 0 | 1130 |
| MT131 | | | | 350 | | | | 0 | 0 | 34 | 202 | 3787 | [symbol] | 2 [symbol] 9 | 0 | [symbol] 774 |
| A [symbol] | | | | 351 | | | | 7840 | 0 | 0 | [symbol] | [symbol] | 0 | 200 | 0 | 0 |
| MT282 | | | | 352 | | | | 250 | 0 | 0 | 0 | 8001 | 0 | 0 | 0 | 18131 |
| ROO [symbol] 3 [symbol] 3 | | | | 353 | | | | 0 | 0 | 134 | 0 | 2870 | 0 | 0 | 0 | 179 |
| TH-19 | | | | 356 | | | | [symbol] | 0 | 0 | 0 | 7868 | 2226 | 0 | 3008 | 17089 |
| [symbol] -3M | | | | 357 | | | | 2843 | 0 | 0 | 0 | 24100 | 16870 | 313 | 37531 | 7453 |
| [symbol] T | | | | 360 | | | | 0 | 0 | 0 | 0 | 1834 | 0 | 0 | [symbol] | 1100 |
| MT213 | | | 60 | | | | | 0 | 0 | 162 | 0 | 13822 | 1275 | 37 | 0 | 43 [symbol] |
| HT [symbol] | | | 62 | | | | | 195 | 0 | 107 | 0 | 5482 | 437 | 0 | 429 | 4 [symbol] 48 |
| HT [symbol] | | | 64 | | | | | 0 | 0 | 0 | 31 | 3341 | 0 | 0 | 0 | 2704 |
| HT134 | | | 54 | | | | | 0 | 0 | 114 | 0 | 3041 | 0 | 0 | 0 | 12179 |
| HT143 | | | 58 | | | | | 337 | 0 | 35 | [symbol] | [symbol] | [symbol] | 0 | 0 | 248 |
| HT170 | | | 60 | | | | | 0 | 0 | 2 [symbol] | 0 | 8332 | 0 | 0 | 0 | 12 [symbol] 50 |
| HT172 | | | 62 | | | | | 0 | 0 | 5 | 0 | 4071 | 0 | 94 | 104 | 2202 |
| HT130 | | | 63 | | | | | 1302 | 0 | 0 | 0 | 3671 | 121 | 0 | 0 | 2 [symbol] |
| HT1 [symbol] | | | 64 | | | | | 0 | 0 | 0 | 701 | 5034 | 0 | 0 | 0 | 2831 |
| HT154 | | | 65 | | | | | 1836 | 0 | 0 | 0 | 8174 | 1 [symbol] 63 | 0 | 0 | 1773 |
| HT140 | | | 66 | | | | | 0 | 0 | 73 | 0 | [symbol] | 0 | 0 | 0 | 2924 |
| HT [symbol] | | | 67 | | | | | 0 | 0 | 0 | 0 | [symbol] | 24 | [symbol] | 343 | 13 [symbol] |
| HT149 | | | 68 | | | | | 0 | 0 | 24 | 5 | 4352 | 0 | 440 | 0 | [symbol] |
| HT143 | | | 69 | | | | | 5 | 0 | 0 | 1176 | 3334 | 0 | [symbol] | 0 | [symbol] |
| HT183 | | | 70 | | | | | [symbol] | 0 | 61 | 0 | 4240 | [symbol] | [symbol] | [symbol] 34 | [symbol] |
| HT148 | | | 71 | | | | | 340 | 0 | 0 | 0 | [symbol] | 0 | 0 | 0 | 3 [symbol] 4 |
| HT227 | | | 72 | | | | | 0 | 0 | 30 | 114 | 4034 | 0 | 0 | 0 | 4727 |
| HT302 | | | 73 | | | | | 420 | 0 | 0 | 123 | 5413 | [symbol] 7 | 32 | 41 [symbol] | [symbol] |
| HT314 | | | 74 | | | | | 429 | 0 | 0 | 0 | 4709 | 6214 | 0 | 0 | [symbol] |
| HT317 | | | 76 | | | | | 243 | 0 | 27 | 0 | [symbol] | 2971 | 0 | 0 | 20750 |
| [symbol] 11 [symbol] | | | 77 | | | | | [symbol] | 0 | 33 | [symbol] | 2199 | [symbol] | 220 | [symbol] | 2212 |
| HT323 | | | 78 | | | | | 83 | 0 | 110 | 0 | [symbol] | [symbol] | 197 | 0 | 5230 |
| HT327 | | | 80 | | | | | 0 | 0 | 124 | 0 | 3771 | 177 [symbol] | [symbol] | 14 | 7320 |
| HT235 | | | 82 | | | | | 0 | 0 | 71 | 0 | [symbol] | [symbol] | 0 | 422 | 17130 |
| HT146 | | | 84 | | | | | 432 | 0 | 120 | 0 | 1141 | 0 | 419 | 0 | 364 |
| HT343 | | | 87 | | | | | 0 | 0 | 0 | 0 | 5314 | [symbol] | 0 | 304 | 21 [symbol] |
| HT311 | | | 170 | | | | | [symbol] | 0 | [symbol] | 0 | 3141 | [symbol] | 234 | 0 | [symbol] |
| HT [symbol] | | | 185 | | | | | 0 | 0 | [symbol] | 0 | 3240 | 0 | 22 [symbol] | 0 | [symbol] |
| HT140 | | | 187 | | | | | 0 | 0 | 0 | 0 | 812 | 0 | 0 | 0 | 303 |
| HT291 | | | 1 [symbol] | | | | | 0 | 0 | 244 | 0 | 2857 | 1134 | 0 | 0 | [symbol] 47 |
| HT372 | | | 191 | | | | | 0 | 0 | [symbol] | 242 | 33 [symbol] | [symbol] 23 | 247 | 0 | 431 |
| TCGP | | | 207 | | | | | 0 | 0 | 0 | 212 | [symbol] | [symbol] | 0 | 811 | [symbol] 17 |
| HT100 | | | 276 | | | | | 131 | 0 | 0 | 0 | 801 | 0 | 202 | 429 | 2730 |
| HT307 | | | 217 | | | | | 0 | 0 | 115 | 0 | 1318 | 0 | 0 | 0 | 4418 |
| HT300 | | | 234 | | | | | 0 | 0 | 45 | 15 | [symbol] | 0 | [symbol] 0 | 827 | 33 [symbol] |
| HT370 | | | 224 | | | | | 1027 | 0 | 22 | 0 | [symbol] | 44 | 134 | 644 | 11858 |
| HT371 | | | 229 | | | | | 0 | 0 | [symbol] | 27 | 4211 | 0 | 0 | 281 | 9410 |
| HT377 | | | 230 | | | | | 405 | 0 | 23 | 0 | 3712 | 779 | 292 | 0 | [symbol] |
| HT342 | | | 236 | | | | | [symbol] | 0 | [symbol] | 0 | [symbol] | 408 | 0 | 627 | 7080 |
| [symbol] | | | 281 | | | | | 0 | 0 | 0 | [symbol] 7 | 0 | 0 | 0 | 0 | 0 |
| HT334 | | | [symbol] | | | | | 0 | 0 | 32 | 0 | 4472 | 0 | 0 | 0 | 32015 |
| HT338 | | | 301 | | | | | 0 | 0 | 0 | 3 | 3010 | 0 | 202 | 0 | 6382 |
| HT342 | | | 315 | | | | | 544 | 0 | 0 | 71 | 2100 | 0 | 0 | 0 | 2019 |
| HT304 | | | 317 | | | | | 0 | 0 | 0 | 102 | 2127 | 19712 | 2 | 0 | [symbol] |
| HT312 | | | 319 | | | | | 2077 | 0 | 0 | 104 | 2504 | 65 | 0 | [symbol] | 6517 |
| HT162 | | | 325 | | | | | 0 | 0 | 10 | 0 | 1411 | 0 | 193 | 0 | 0 |
| HT [symbol] | | | 334 | | | | | 0 | 0 | 0 | 0 | 6212 | 294 | 0 | 182 | 4313 |
| HT137 | | | 3 [symbol] 0 | | | | | [symbol] | 0 | 6 | 324 | 2314 | 426 | 0 | 374 | 1 [symbol] |
| T-410 | 143 | | | | | | | [symbol] | 0 | 0 | 0 | 4107 | [symbol] | 253 | [symbol] | [symbol] |
| MDA-N | 141 | | | | | | | 22 [symbol] | 0 | 49 | 0 | [symbol] | 0 | [symbol] | 13 [symbol] | [symbol] |
| MOA- [symbol] -4.34 | 108 | | | | | | | 1148 | 0 | 68 | 130 | [symbol] | 814 | [symbol] | 7 [symbol] | [symbol] |
| MOA- [symbol] -231 | 157 | | | | | | | 3 [symbol] 1 | 0 | 0 | 103 | 4223 | 381 | 200 | 141 | [symbol] |
| Ha [symbol] | 153 | | | | | | | [symbol] | 0 | 84 | 0 | 4775 | 0 | 870 | 1534 | 13305 |
| MCF-7/AC [symbol] | 153 | | | | | | | [symbol] | 0 | 0 | [symbol] | [symbol] | 0 | 110 | [symbol] | [symbol] |
| MCF/ | 151 | | | | | | | 3033 | 0 | [symbol] | 0 | [symbol] | 0 | [symbol] 2 | 0 | [symbol] |
| M [symbol] | 149 | | | | | | | 0 | 0 | [symbol] | 0 | [symbol] | 0 | [symbol] 3 | 0 | [symbol] |
| LIACC-257 | 147 | | | | | | | 0 | 0 | 0 | 0 | [symbol] | 0 | 0 | 813 | [symbol] |
| LIACC- [symbol] | 145 | | | | | | | 0 | 0 | 47 | [symbol] | [symbol] | 0 | [symbol] | [symbol] | [symbol] |
| [symbol] | 144 | | | | | | | [symbol] | 0 | 101 | 0 | [symbol] | 0 | [symbol] | [symbol] | [symbol] |
| LO-31 | 143 | | | | | | | 756 | 0 | 0 | 0 | [symbol] | 0 | [symbol] | [symbol] | [symbol] |
| [symbol] | 142 | | | | | | | [symbol] | 0 | 14 | 0 | [symbol] | 0 | [symbol] | [symbol] | [symbol] |
| [symbol] -12 | 141 | | | | | | | [symbol] | 0 | 0 | 29 | [symbol] | 0 | 437 | [symbol] | [symbol] |
| [symbol] | 140 | | | | | | | 0 | 0 | 4 | 0 | [symbol] | 0 | 177 | [symbol] | [symbol] |
| NCT-1 [symbol] | 139 | | | | | | | 0 | 0 | 0 | 0 | [symbol] | 0 | [symbol] | [symbol] | [symbol] |
| [symbol] | 139 | | | | | | | 1 [symbol] | 0 | 0 | 0 | [symbol] | 0 | 3 [symbol] | [symbol] | [symbol] |
| C₂O [symbol] O20 [symbol] | 137 | | | | | | | [symbol] 2 | 0 | 232 | [symbol] | [symbol] | 0 | 311 | [symbol] | [symbol] |
| LOX [symbol] | 138 | | | | | | | [symbol] | 0 | 0 | 121 | [symbol] | 0 | 0 | [symbol] | [symbol] |
| [symbol] | 135 | | | | | | | [symbol] | 0 | 37 | 0 | [symbol] | 0 | [symbol] | [symbol] | [symbol] |
| [symbol] | 134 | | | | | | | 1 [symbol] | 0 | 0 | 10 | [symbol] | 0 | 304 | [symbol] | [symbol] |
| HCT [symbol] | 133 | | | | | | | [symbol] | 0 | 107 | 104 | [symbol] | 0 | 204 | [symbol] | [symbol] |
| [symbol] | 132 | | | | | | | [symbol] 791 | 0 | 7 | 110 | [symbol] | 0 | 180 | [symbol] | [symbol] |
| HCC- [symbol] | 131 | | | | | | | 1371 | 0 | 0 | 0 | [symbol] | 0 | 370 | [symbol] | [symbol] |
| AC [symbol] | 130 | | | | | | | 0 | 0 | 0 | 104 | [symbol] | 0 | 2 [symbol] | [symbol] | [symbol] |
| C-2 [symbol] | 129 | | | | | | | 0 | 0 | 0 | 0 | [symbol] | 0 | 102 | [symbol] | [symbol] |
| [symbol] | 129 | | | | | | | [symbol] | 0 | 11 | 212 | [symbol] | 0 | 220 | [symbol] | [symbol] |
| [symbol] | 127 | | | | | | | 454 | 0 | 0 | 82 | [symbol] | 0 | [symbol] | [symbol] | [symbol] |
| C [symbol] | 126 | | | | | | | 0 | 0 | 132 | 160 | [symbol] | 0 | [symbol] | [symbol] | [symbol] |
| [symbol] | 128 | | | | | | | [symbol] | 0 | 2 | 0 | [symbol] | 0 | [symbol] | [symbol] | [symbol] |
| [symbol] | 124 | | | | | | | 2 [symbol] | 0 | 124 | 245 | [symbol] | 0 | [symbol] | [symbol] | [symbol] |
| R [symbol] 8228 | 121 | | | | | | | 0 | 0 | 0 | 64 | [symbol] | 0 | 0 | [symbol] | [symbol] |
| [symbol] | 122 | | | | | | | 0 | 0 | 0 | [symbol] | [symbol] | 0 | 732 | [symbol] | [symbol] |
| HS-40 | 12 [symbol] | | | | | | | 0 | 0 | 0 | 104 | [symbol] | 0 | 102 | [symbol] | [symbol] |
| MCL, T-4 | 120 | | | | | | | [symbol] | 0 | 24 | 0 | [symbol] | 0 | [symbol] | [symbol] | [symbol] |
| OVCAR-5 | 119 | | | | | | | 576 | 0 | 14 | 30 | [symbol] | 0 | 203 | [symbol] | [symbol] |
| X-562 [symbol] | 119 | | | | | | | 0 | 0 | 0 | 0 | [symbol] | 0 | 83 | [symbol] | [symbol] |
| OVCAR [symbol] -4 | 117 | | | | | | | [symbol] | 0 | 0 | 0 | [symbol] | 0 | [symbol] | [symbol] | [symbol] |
| CC [symbol] -CEM | 118 | | | | | | | [symbol] | 0 | 70 | 0 | [symbol] | 0 | 372 | [symbol] | [symbol] |
| OVCAR-3 | 115 | | | | | | | 2334 | 0 | [symbol] | 0 | [symbol] | 0 | 302 | [symbol] | [symbol] |
| 5F-539 [symbol] | 114 | | | | | | | 941 | 0 | 2 | 0 | [symbol] | 0 | 1013 | [symbol] | [symbol] |
| HCF- [symbol] | 113 | | | | | | | 527 | 0 | 0 | [symbol] | [symbol] | 0 | 130 | [symbol] | [symbol] |
| SF- [symbol] | 112 | | | | | | | 678 | 0 | 0 | 210 | [symbol] | 0 | 0 | [symbol] | [symbol] |
| ASCMATCC | 111 | | | | | | | 1174 | 0 | 0 | 193 | [symbol] | 0 | 74 | [symbol] | [symbol] |
| SF-Z [symbol] | 110 | | | | | | | [symbol] | 0 | 0 | 112 | [symbol] | 0 | 387 | [symbol] | [symbol] |
| NC [symbol] | 100 | | | | | | | 0 | 0 | 21 | 0 | [symbol] | 0 | 854 | [symbol] | [symbol] |
| U251 | 104 | | | | | | | [symbol] | 0 | 0 | 474 | [symbol] | 0 | 102 | [symbol] | [symbol] |
| NCI- [symbol] | 107 | | | | | | | 2 [symbol] | 0 | 221 | 47 | [symbol] | 0 | [symbol] 3 | [symbol] | [symbol] |
| S [symbol] -7 [symbol] | 104 | | | | | | | [symbol] | 0 | 76 | 3 | [symbol] | 0 | 231 | 1213 | [symbol] |
| MC [symbol] | 105 | | | | | | | 0 | 0 | 0 | 20 | [symbol] | 0 | 110 | 0 | [symbol] |
| S [symbol] - [symbol] | 104 | | | | | | | 20 | 0 | 63 | 0 | [symbol] | 0 | 110 | 0 | [symbol] |
| MC3-H228 [symbol] | 103 | | | | | | | [symbol] | 0 | 19 | 0 | [symbol] | 0 | 110 | 0 | [symbol] |
| SV-OV-3 | 102 | | | | | | | [symbol] | 0 | 0 | 0 | [symbol] | 0 | 110 | 2000 | [symbol] |
| MCF-23 [symbol] | 101 | | | | | | | 200 | 0 | 142 | 0 | [symbol] | 0 | 110 | 1710 | [symbol] |
| [symbol] | 100 | | | | | | | 0 | 0 | 0 | 0 | 0 | 4775 | 0 | 0 | [symbol] |
| [symbol] | [symbol] | | | | | | | [symbol] | 0 | 14 | 14 | [symbol] | 3052 | 0 | 20 | [symbol] |
| OVCAR- [symbol] | 99 | | | | | | | 0 | 0 | 72 | 0 | [symbol] | [symbol] | 0 | 111 | 2 [symbol] |
| HCF-43 [symbol] | 87 | | | | | | | 0 | 0 | 0 | 0 | [symbol] | 0 | 110 | 446 | 4534 |
| [symbol] 12 | 44 | | | | | | | 11 [symbol] | 0 | 123 | 112 | 2977 | [symbol] | 0 | 1226 | 2044 |
| [symbol] 17 | 47 | | | | | | | 2736 | 0 | 112 | 0 | 4152 | [symbol] | 0 | 747 | 4422 |
| [symbol] 10 | 46 | | | | | | | 1043 | 0 | 0 | 329 | 3112 | [symbol] | 254 | 0 | 2 [symbol] |
| TCCP | 24 | | | | | | | 0 | 0 | 114 | 0 | 3352 | [symbol] 36 | 254 | 311570 | 311 [symbol] |
| [symbol] -1 | | | | | | | wt | 487 | 106 | 37 | 0 | 0 | 0 | 0 | 0 | 0 |
| AS [symbol] -2 | | | | | | | wt | 317 | [symbol] | 193 | 0 | 0 | 0 | 0 | 0 | 0 |
| AS [symbol] -4 | | | | | | | wt | 0 | 109 | 92 | 0 | 0 | 0 | 0 | 0 | 0 |
| [symbol] -5 | | | | | | | wt | 321 | 234 | 44 | 0 | 0 | 0 | 0 | 0 | 0 |
| [symbol] -7 | | | | | | | [symbol] | 5881 | [symbol] | 265 | 0 | 0 | 0 | 0 | 0 | 0 |
| BXVx-3 | | | | | | | [symbol] | 2147 | [symbol] | 0 | 0 | 0 | 0 | 0 | 0 | 0 |
| BXVx-4 | | | | | | | [symbol] | 2885 | 0 | [symbol] | 0 | 0 | 0 | 0 | 0 | 0 |
| BXVx-3 | | | | | | | [symbol] | 1030 | 0 | 0 | 0 | 0 | 0 | 0 | 0 | 0 |
| BXVx-5 | | | | | | | [symbol] | 0 | 1623 | 0 | 0 | 0 | 0 | 0 | 0 | 0 |
| BXVx-7 | | | | | | | wt | 0 | 0 | 0 | 0 | 0 | 0 | 0 | 0 | 0 |
| MCF-7-1 | | | | | | | wt | 403 | 0 | [symbol] | 0 | 0 | 0 | 0 | 0 | 0 |
| MCF-7-2 | | | | | | | wt | 190 | 0 | 116 | 0 | 0 | 0 | 0 | 0 | 0 |
| MCF-7-4 | | | | | | | wt | 3124 | 0 | 0 | 0 | 0 | 0 | 0 | 0 | 0 |
| MCF-7- [symbol] | | | | | | | wt | [symbol] | 0 | 1213 | 0 | 0 | 0 | 0 | 0 | 0 |
| MCF-7-7 | | | | | | | [symbol] | 0 | 0 | 135 | 0 | 0 | 0 | 0 | 0 | 0 |
| AC [symbol] -RES-1 | | | | | | | [symbol] | [symbol] 24 | 278 | 290 | 0 | 0 | 0 | 0 | 0 | 0 |
| AC [symbol] -RES-3 | | | | | | | [symbol] | 4757 | 572 | 109 | 0 | 0 | 0 | 0 | 0 | 0 |
| AC [symbol] -RES-4 | | | | | | | [symbol] | 436 | 241 | 84 | 0 | 0 | 0 | 0 | 0 | 0 |
| AC [symbol] -RES-5 | | | | | | | [symbol] | 7 [symbol] | [symbol] 1 | 11 | 0 | 0 | 0 | 0 | 0 | 0 |
| AC [symbol] -RES-7 | | | | | | | wt | 2513 | 0 | 176 | 0 | 0 | 0 | 0 | 0 | 0 |
| WI 34-1 | | | | | | | wt | 1 [symbol] 9 | 0 | [symbol] | 0 | 0 | 0 | 0 | 0 | 0 |
| WI 34-3 | | | | | | | wt | 0 | 0 | 0 | 0 | 0 | 0 | 0 | 0 | 0 |
| WI 34-4 | | | | | | | wt | 30 [symbol] | 0 | 37 | 0 | 0 | 0 | 0 | 0 | 0 |
| WI 34-6 | | | | | | | wt | [symbol] 12 | 365 | 43 | 0 | 0 | 0 | 0 | 0 | 0 |
| WI 34-7 | | | | | | | MPV [symbol] | [symbol] 12 | 0 | 7 | 0 | 0 | 0 | 0 | 0 | 0 |
| [symbol] -1 | | | | | | | MPV [symbol] | 0 | 0 | 60 | 0 | 0 | 0 | 0 | 0 | 0 |
| [symbol] -3 | | | | | | | MPV [symbol] | [symbol] 2 | [symbol] | 115 | 0 | 0 | 0 | 0 | 0 | 0 |
| [symbol] -4 | | | | | | | MPV [symbol] | [symbol] | [symbol] | 172 | 0 | 0 | 0 | 0 | 0 | 0 |
| [symbol] -6 | | | | | | | MPV [symbol] | [symbol] 47 | 0 | 7 | 0 | 0 | 0 | 0 | 0 | 0 |
| [symbol] -7 | | | | | | | [symbol] | 42 [symbol] | 0 | 27 | 0 | 0 | 0 | 0 | 0 | 0 |
| HI [symbol] -1 | | | | | | | [symbol] | 0 | 0 | 183 | 0 | 0 | 0 | 0 | 0 | 0 |
| HI [symbol] -3 | | | | | | | [symbol] | 1149 | [symbol] | 34 | 0 | 0 | 0 | 0 | 0 | 0 |
| HI [symbol] -4 | | | | | | | [symbol] | 1154 | 0 | 104 | 0 | 0 | 0 | 0 | 0 | 0 |
| HI [symbol] -5 | | | | | | | [symbol] | 819 | 341 | 71 | 0 | 0 | 0 | 0 | 0 | 0 |
| HI [symbol] -7 | | | | | | | wt | 0 | 2430 | 0 | 0 | 0 | 0 | 0 | 0 | 0 |
| [symbol] -2 | | | | | | | wt | 751 | 0 | 205 | 0 | 0 | 0 | 0 | 0 | 0 |
| B [symbol] -2 | | | | | | | [symbol] | 21741 | [symbol] | 206 | 0 | 0 | 0 | 0 | 0 | 0 |
| HCT-114-1 | | | | | | | wt | 849 | 256 | 430 | 0 | 0 | 0 | 0 | 0 | 0 |
| HCT-114-2 | | | | | | | wt | 8204 | 261 | [symbol] | 0 | 0 | 0 | 0 | 0 | 0 |
| HT29-2 | | | | | | | wt | [symbol] 7 | [symbol] 202 | 0 | 0 | 0 | 0 | 0 | 0 | 0 |
| SFC30-1 | | | | | | | wt | 1107 | 261 | 0 | 0 | 0 | 0 | 0 | 0 | 0 |
| SFC30-2 | | | | | | | wt | [symbol] 3 | 302 | 71 | 0 | 0 | 0 | 0 | 0 | 0 |
| SF- [symbol] -1 | | | | | | | wt | [symbol] 74 | [symbol] 4 | 84 | 130 | 0 | 0 | 0 | 0 | 0 |
| SF [symbol] -2 | | | | | | | [symbol] | 11314 | 4311 | 295 | 0 | 0 | 0 | 0 | 0 | 0 |
| OVC-1 | | | | | | | [symbol] | [symbol] | 701 | 0 | 0 | 0 | 0 | 0 | 0 | 0 |
| OVC-2 | | | | | | | wt | 0 | 0 | 0 | 0 | 0 | 0 | 0 | 0 | 0 |
| OVC-1 | | | | | | | wt | [symbol] 05 | 0 | 0 | 0 | 0 | 0 | 0 | 0 | 0 |
| OVC-2 | | | | | | | [symbol] | [symbol] 7 | [symbol] | 0 | 0 | 0 | 0 | 0 | 0 | 0 |
| HCF-7-2 | | | | | | | wt | 21 [symbol] | 0 | 43 | 0 | 0 | 0 | 0 | 0 | 0 |
| [symbol] - [symbol] -2 | | | | | | | [symbol] | [symbol] | 747 | [symbol] | 0 | 0 | 0 | 0 | 0 | 0 |
| [symbol] -2 | | | | | | | HPV [symbol] | 2741 | 0 | [symbol] | 0 | 0 | 0 | 0 | 0 | 0 |
| [symbol] -1 | | | | | | | [symbol] | [symbol] 3 | 1075 | [symbol] | 0 | 0 | 0 | 0 | 0 | 0 |
| [symbol] -2 | | | | | | | [symbol] | [symbol] 3 | 714 | [symbol] | 0 | 0 | 0 | 0 | 0 | 0 |
| C3 [symbol] -1 | | | | | | | [symbol] | 322 | 571 | 0 | 0 | 0 | 0 | 0 | 0 | 0 |
| C33A-2 | | | | | | | [symbol] | 315 | 0 | 57 | 0 | 0 | 0 | 0 | 0 | 0 |
| U3O [symbol] -1 | | | | | | | [symbol] | [symbol] 4 [symbol] | 0 | 75 | 0 | 0 | 0 | 0 | 0 | 0 |
| U3 [symbol] -2 | | | | | | | [symbol] | 8301 | 1141 | 0 | 0 | 0 | 0 | 0 | 0 | 0 |
| H [symbol] -1 | | | | | | | [symbol] | 3542 | 1206 | 00 | 0 | 0 | 0 | 0 | 0 | 0 |
| [symbol] -2 | | | | | | | [symbol] | 2 [symbol] | [symbol] | 46 | 0 | 0 | 0 | 0 | 0 | 0 |
| WI [symbol] -2 | | | | | | | wt | 430 [symbol] | 734 | 18 | 0 | 0 | 0 | 0 | 0 | 0 |
| [symbol] - [symbol] | | | | | | | wt | 0 | 631 | 21 | 0 | 0 | 0 | 0 | 0 | 0 |
| [symbol] -1 | | | | | | | wt | 513 | 0 | 72 | 0 | 0 | 0 | 0 | 0 | 0 |
| [symbol] -2 | | | | | | | | 3 [symbol] | 0 | [symbol] | 0 | 0 | 0 | 0 | 0 | 0 |
| [symbol] -3 | | | | | | | | [symbol] | 0 | 0 | 0 | 0 | 0 | 0 | 0 | 0 |
| [symbol] -4 | | | | | | | | [symbol] | [symbol] | 0 | 0 | 0 | 0 | 0 | 0 | 0 |
| [symbol] -5 | | | | | | | | [symbol] | 0 | 0 | 0 | 0 | 0 | 0 | 0 | 0 |
| [symbol] -6 | | | | | | | | [symbol] | 739 | 311 | 0 | 0 | 0 | 0 | 0 | 0 |
| [symbol] -7 | | | | | | | | [symbol] | 0 | 37 | 0 | 0 | 0 | 0 | 0 | 0 |
| [symbol] -8 | | | | | | | | [symbol] | 0 | 0 | 0 | 0 | 0 | 0 | 0 | 0 |
| [symbol] | | | | [symbol] | | | | [symbol] | [symbol] | 157 | 1273 | 872 | 1279 | 1930 | [symbol] | [symbol] |
| [symbol] | | | | 160 | | | | 233 | [symbol] | 227 | 273 | 272 | 8628 | [symbol] | [symbol] | 23879 |
| [symbol] | | | | 140 | | | | [symbol] | [symbol] | 0 | 1074 | 232 | 100 | 2001 | [symbol] | [symbol] |
| [symbol] | | | | 171 | | | | 9 | 1863 | 0 | 234 | 13 | 137 | 179 | 138 | [symbol] |
| [symbol] | | | | 181 | | | | 279 | 3104 | 0 | 24 | 0 | 0 | [symbol] | 0 | 13074 |
| [symbol] | | | | 183 | | | | 127 | 833 | 0 | [symbol] | 0 | 0 | 643 | 230 | [symbol] |
| [symbol] | | | | 184 | | | | 0 | [symbol] | 0 | 104 | 0 | 1428 | 1643 | [symbol] | [symbol] |
| [symbol] | | | | 186 | | | | 237 | 3283 | 8277 | 0 | 0 | 238 | 2918 | [symbol] | 17778 |
| [symbol] | | | | 188 | | | | 0 | 8583 | 0 | 259 | 0 | 1158 | 2807 | 14303 | 28547 |
| uncc.Q | | | | 200 | | | | 1432 | [symbol] | 0 | 252 | 0 | 475 | 2323 | 2875 | 17274 |
| [symbol] | | | | 202 | | | | 185 | 2223 | 245 | 0 | 11 | 281 | 1444 | [symbol] | [symbol] |
| UTCS [symbol] | | | | 204 | | | | 9 | 7812 | 0 | 704 | 9 | 8 | 1820 | 617 | [symbol] |
| [symbol] | | | | [symbol] | | | | 198 | 9 | 0 | 34 | 123 | 850 | 673 | [symbol] | [symbol] |
| [symbol] | | | | [symbol] | | | | 0 | 18176 | 0 | 423 | 43 | 0 | 2343 | 23131 | [symbol] |
| CCL 137 RAX 321 [symbol] | | | | 214 | | | | 273 | 1848 | 0 | 290 | 0 | 287 | 1447 | [symbol] | 73444 |
| WL37 73 [symbol] | | | | 219 | | | | 271 | 3187 | 1980 | 0 | 34 | 423 | 3283 | 0 | 61474 |
| [symbol] 1 | | | | 220 | | | | 0 | [symbol] | 0 | 31 | [symbol] | 0 | [symbol] | 63 | [symbol] |
| [symbol] 2 | | | | 221 | | | | 2284 | 2813 | 4 | 6 | 0 | 423 | 2710 | 154 | 46200 |
| [symbol] 4 | | | | 222 | | | | 705 | 2838 | 0 | 113 | 0 | 322 | 2128 | 41 | 41277 |
| [symbol] | | | | 223 | | | | 827 | [symbol] | 0 | 37 | 0 | 1300 | 2312 | 88 | 28345 |
| [symbol] | | | | 241 | | | | 0 | 2342 | 0 | 218 | 0 | 0 | 1773 | 13338 | [symbol] |
| [symbol] | | | | 242 | | | | [symbol] | [symbol] | 782 | 483 | 461 | 2874 | 3182 | 38418 | [symbol] |
| [symbol] | | | | 243 | | | | 0 | 13683 | 0 | 328 | 73 | 3120 | [symbol] | [symbol] | [symbol] |
| [symbol] | | | | 244 | | | | 0 | 14211 | [symbol] | 23 | 431 | 2402 | 3000 | 13000 | [symbol] |
| [symbol] | | | | 245 | | | | 150 | 13043 | 577 | 2005 | 187 | 3423 | 4323 | [symbol] | [symbol] |
| [symbol] | | | | 246 | | | | 0 | 1320 | 480 | 179 | 0 | 324 | 2372 | 18372 | [symbol] |
| [symbol] | | | | 247 | | | | [symbol] | 3460 | 0 | 819 | 384 | 831 | 1981 | 2383 | 18071 |
| [symbol] | | | | 248 | | | | 0 | 1314 | 323 | 0 | 23 | 204 | [symbol] | [symbol] | 17287 |
| [symbol] | | | | 249 | | | | 0 | 7307 | 0 | 214 | 120 | 1020 | 2434 | 7704 | [symbol] |
| [symbol] | | | | 250 | | | | 0 | 18785 | 309 | 1450 | 0 | 2338 | [symbol] | [symbol] | 27342 |
| [symbol] | | | | 251 | | | | 223 | 314 | 1399 | 0 | 189 | 473 | 804 | 1431 | 7813 |
| [symbol] | | | | 252 | | | | 372 | 3404 | 0 | 0 | 0 | 143 | 547 | 1123 | 1000 |
| [symbol] | | | | 253 | | | | 2503 | [symbol] | 1811 | 825 | 87 | 1738 | 3343 | 12200 | [symbol] |
| [symbol] | | | | 254 | | | | 0 | 5112 | 804 | 178 | 804 | 2335 | [symbol] | [symbol] | 18317 |
| [symbol] | | | | 255 | | | | 8 | 3304 | 824 | 733 | 0 | 1122 | 1380 | [symbol] | 14180 |
| [symbol] | | | | 256 | | | | 273 | 17258 | 435 | 834 | 61 | [symbol] | [symbol] | 30147 | [symbol] |
| [symbol] | | | | 257 | | | | 1134 | 8352 | 758 | 718 | 84 | 2332 | 3044 | [symbol] | 31784 |
| [symbol] | | | | 258 | | | | 8 | 2305 | 0 | 19 | 0 | 0 | [symbol] | 4002 | 13814 |
| [symbol] | | | | 259 | | | | 83 | 3327 | 0 | 200 | 0 | 1000 | 3742 | 8318 | 17044 |
| [symbol] | | | | 260 | | | | 136 | 3811 | 0 | 48 | 123 | 1305 | 1144 | 3811 | [symbol] |
| [symbol] | | | | 261 | | | | 3832 | 12225 | 0 | 834 | 152 | 7473 | [symbol] | [symbol] | [symbol] |
| [symbol] | | | | 262 | | | | 0 | 7037 | 0 | 384 | 0 | 1713 | 1981 | 8411 | [symbol] |
| [symbol] | | | | 263 | | | | 847 | 8209 | 1348 | 1885 | 30 | [symbol] | 1374 | [symbol] | [symbol] |
| [symbol] | | | | 264 | | | | 9 | 4275 | 0 | 428 | 0 | 1320 | 2304 | 3184 | [symbol] |
| [symbol] | | | | 265 | | | | 0 | 2838 | 0 | 0 | 214 | 804 | 717 | 1178 | 17725 |
| [symbol] | | | | 267 | | | | 829 | [symbol] | [symbol] | [symbol] | 834 | [symbol] | [symbol] | 78777 | 80530 |
| [symbol] | | | | 259 | | | | 144 | 8376 | 477 | 843 | 0 | [symbol] | 1857 | 1143 | 24183 |
| [symbol] | | | | 270 | | | | 14 | 4483 | 0 | 0 | 0 | 891 | 2124 | 281 | 41001 |
| [symbol] | | | | 271 | | | | 278 | 7085 | 0 | 354 | 02 | 1290 | 1838 | [symbol] | [symbol] |
| [symbol] | | | | 273 | | | | 0 | [symbol] | 0 | 378 | 0 | 3785 | 8025 | [symbol] | [symbol] |
| [symbol] | | | | [symbol] | | | | 0 | 20754 | 17233 | 317 | 234 | 8784 | 4273 | 38084 | 70087 |
| [symbol] | | | | 300 | | | | 783 | 3081 | 0 | 1454 | 0 | 2334 | 2492 | 237 | 31817 |
| [symbol] | | | | 313 | | | | 834 | 1812 | 0 | 247 | 0 | 1340 | 2042 | 4181 | 30714 |
| [symbol] | | | | 322 | | | | 1441 | [symbol] | 0 | 0 | [symbol] | 2312 | 8180 | [symbol] | [symbol] |
| [symbol] | | | | 323 | | | | 404 | [symbol] | 0 | 0 | 0 | 2030 | 3024 | [symbol] | 43021 |
| [symbol] | | | | 324 | | | | 1327 | 40673 | 0 | 0 | [symbol] | [symbol] | [symbol] | [symbol] | [symbol] |
| [symbol] | | | | [symbol] | | | | 672 | [symbol] | [symbol] | [symbol] | 0 | [symbol] | [symbol] | [symbol] | [symbol] |
| [symbol] | | | | 237 | | | | 0 | [symbol] | 0 | [symbol] | [symbol] | [symbol] | 1717 | [symbol] | [symbol] |
| [symbol] | | | | 238 | | | | 1103 | [symbol] | 1217 | [symbol] | 1217 | [symbol] | [symbol] | [symbol] | [symbol] |
| [symbol] | | | | 239 | | | | 132 | [symbol] | 1171 | [symbol] | [symbol] | [symbol] | [symbol] | [symbol] | [symbol] |
| [symbol] | | | | 240 | | | | [symbol] | [symbol] | 0 | [symbol] | 0 | [symbol] | [symbol] | [symbol] | [symbol] |
| [symbol] | | | | 241 | | | | 177 | [symbol] | [symbol] | [symbol] | 2330 | [symbol] | [symbol] | [symbol] | [symbol] |
| [symbol] | | | | 242 | | | | [symbol] | [symbol] | 0 | [symbol] | [symbol] | [symbol] | [symbol] | [symbol] | [symbol] |
| [symbol] | | | | 245 | | | | 25 | [symbol] | 0 | [symbol] | [symbol] | [symbol] | [symbol] | [symbol] | [symbol] |
| [symbol] | | | | 246 | | | | 1001 | [symbol] | [symbol] | [symbol] | 0 | 1412 | 6254 | [symbol] | [symbol] |
| [symbol] | | | | 247 | | | | [symbol] | [symbol] | 0 | [symbol] | 0 | 0 | [symbol] | [symbol] | [symbol] |
| [symbol] | | | | 248 | | | | [symbol] | [symbol] | 0 | 77 | 114 | 0 | [symbol] | [symbol] | [symbol] |
| [symbol] | | | | 249 | | | | [symbol] | 11062 | 0 | 408 | 0 | [symbol] | [symbol] | [symbol] | [symbol] |
| [symbol] | | | | 250 | | | | [symbol] | [symbol] | 0 | 0 | 0 | 723 | 2737 | [symbol] | [symbol] |
| [symbol] | | | | 251 | | | | [symbol] | [symbol] | 0 | [symbol] | [symbol] | [symbol] | [symbol] | [symbol] | [symbol] |
| [symbol] | | | | 252 | | | | 724 | [symbol] | [symbol] | 0 | 0 | [symbol] | [symbol] | [symbol] | [symbol] |
| [symbol] | | | | 253 | | | | 0 | [symbol] | 0 | 114 | 73 | [symbol] | 2720 | 7230 | [symbol] |
| [symbol] | | | | 255 | | | | [symbol] | [symbol] | [symbol] | [symbol] | [symbol] | [symbol] | [symbol] | [symbol] | [symbol] |
| [symbol] | | | | 257 | | | | 2111 | [symbol] | [symbol] | [symbol] | [symbol] | [symbol] | [symbol] | [symbol] | [symbol] |
| [symbol] | | | | 258 | | | | 0 | [symbol] | 0 | [symbol] | [symbol] | [symbol] | [symbol] | [symbol] | [symbol] |
| [symbol] | | | 50 | | | | | 0 | [symbol] | 47 | 0 | 0 | [symbol] | 1700 | 28 | [symbol] |
| [symbol] | | | 52 | | | | | [symbol] | [symbol] | 0 | 0 | 272 | 783 | 2541 | 7541 | [symbol] |
| [symbol] | | | 54 | | | | | [symbol] | [symbol] | 0 | 4 | [symbol] | 0 | [symbol] | [symbol] | [symbol] |
| [symbol] | | | 56 | | | | | 0 | [symbol] | 0 | [symbol] | [symbol] | [symbol] | 2131 | 2121 | [symbol] |
| [symbol] | | | 58 | | | | | [symbol] | [symbol] | [symbol] | 0 | [symbol] | [symbol] | [symbol] | [symbol] | [symbol] |
| [symbol] | | | 60 | | | | | 31 | [symbol] | 0 | 252 | 0 | [symbol] | 4324 | [symbol] | [symbol] |
| [symbol] | | | 62 | | | | | 0 | [symbol] | 0 | 0 | 17 | [symbol] | [symbol] | 0 | [symbol] |
| [symbol] | | | 63 | | | | | 0 | 2523 | 0 | 0 | 43 | [symbol] | 1070 | [symbol] | [symbol] |
| [symbol] | | | 64 | | | | | 31 | [symbol] | 0 | 0 | 143 | 343 | [symbol] | 800 | [symbol] |
| [symbol] | | | 65 | | | | | 0 | 1277 | [symbol] | 0 | [symbol] | [symbol] | [symbol] | 0 | [symbol] |
| [symbol] | | | 66 | | | | | [symbol] | [symbol] | 0 | [symbol] | 0 | [symbol] | [symbol] | [symbol] | [symbol] |
| [symbol] | | | 67 | | | | | 427 | [symbol] | 0 | 0 | 229 | [symbol] | 455 | [symbol] | [symbol] |
| [symbol] | | | 68 | | | | | [symbol] | [symbol] | [symbol] | 122 | 0 | [symbol] | [symbol] | [symbol] | [symbol] |
| [symbol] | | | 69 | | | | | 2067 | 246 | 0 | 0 | 0 | [symbol] | [symbol] | 42 | [symbol] |
| [symbol] | | | 70 | | | | | 0 | [symbol] | [symbol] | [symbol] | 0 | [symbol] | [symbol] | [symbol] | [symbol] |
| [symbol] | | | 71 | | | | | 1063 | 3301 | [symbol] | 0 | 74 | 3282 | 1823 | 230 | [symbol] |
| [symbol] | | | 72 | | | | | 0 | [symbol] | 0 | [symbol] | [symbol] | [symbol] | [symbol] | 257 | [symbol] |
| [symbol] | | | 73 | | | | | 0 | [symbol] | 0 | 430 | 112 | 0 | 2081 | [symbol] | [symbol] |
| [symbol] | | | 74 | | | | | 0 | [symbol] | [symbol] | 0 | 41 | 279 | 1765 | [symbol] | [symbol] |
| [symbol] | | | [symbol] | | | | | 275 | [symbol] | 0 | 2002 | [symbol] | [symbol] | [symbol] | [symbol] | [symbol] |
| [symbol] | | | 77 | | | | | 6 | 3161 | [symbol] | [symbol] | [symbol] | [symbol] | 2011 | [symbol] | [symbol] |
| [symbol] | | | [symbol] | | | | | [symbol] | [symbol] | [symbol] | 1120 | [symbol] | [symbol] | [symbol] | [symbol] | [symbol] |
| [symbol] | | | 80 | | | | | [symbol] | [symbol] | 0 | [symbol] | 41 | [symbol] | 1737 | [symbol] | [symbol] |
| [symbol] | | | 82 | | | | | 0 | 7064 | 112 | 1 | 201 | 2486 | 2254 | [symbol] | 70437 |
| [symbol] | | | 85 | | | | | [symbol] | [symbol] | [symbol] | 142 | 0 | 0 | [symbol] | [symbol] | 12152 |
| [symbol] | | | 87 | | | | | [symbol] | [symbol] | [symbol] | [symbol] | 203 | [symbol] | 1570 | 2273 | [symbol] |
| [symbol] | | | 170 | | | | | 1002 | 3027 | 0 | 0 | 0 | [symbol] | [symbol] | [symbol] | [symbol] |
| [symbol] | | | [symbol] | | | | | [symbol] | [symbol] | [symbol] | [symbol] | 0 | 0 | [symbol] | [symbol] | [symbol] |
| [symbol] | | | [symbol] | | | | | 0 | [symbol] | 0 | [symbol] | 70 | [symbol] | [symbol] | 0 | [symbol] |
| [symbol] | | | 100 | | | | | [symbol] | 7336 | 0 | 37 | 0 | 0 | [symbol] | 0 | [symbol] |
| [symbol] | | | [symbol] | | | | | [symbol] | [symbol] | 0 | 130 | 104 | [symbol] | [symbol] | [symbol] | [symbol] |
| [symbol] | | | 207 | | | | | [symbol] | [symbol] | [symbol] | [symbol] | [symbol] | [symbol] | [symbol] | [symbol] | [symbol] |
| [symbol] | | | [symbol] | | | | | 0 | [symbol] | [symbol] | [symbol] | [symbol] | [symbol] | [symbol] | [symbol] | [symbol] |
| [symbol] | | | 217 | | | | | 0 | 3175 | 0 | 0 | 210 | [symbol] | [symbol] | [symbol] | [symbol] |
| [symbol] | | | 224 | | | | | [symbol] | [symbol] | [symbol] | [symbol] | [symbol] | [symbol] | [symbol] | [symbol] | [symbol] |
| [symbol] | | | 226 | | | | | [symbol] | [symbol] | [symbol] | [symbol] | [symbol] | [symbol] | [symbol] | [symbol] | [symbol] |
| [symbol] | | | 228 | | | | | [symbol] | [symbol] | [symbol] | [symbol] | [symbol] | [symbol] | [symbol] | [symbol] | [symbol] |
| [symbol] | | | 230 | | | | | [symbol] | [symbol] | [symbol] | [symbol] | [symbol] | [symbol] | [symbol] | [symbol] | [symbol] |
| [symbol] | | | 234 | | | | | [symbol] | [symbol] | [symbol] | [symbol] | [symbol] | [symbol] | [symbol] | [symbol] | [symbol] |
| [symbol] | | | [symbol] | | | | | [symbol] | [symbol] | [symbol] | [symbol] | [symbol] | [symbol] | [symbol] | [symbol] | [symbol] |
| [symbol] | | | [symbol] | | | | | [symbol] | [symbol] | [symbol] | [symbol] | [symbol] | [symbol] | [symbol] | [symbol] | [symbol] |
| [symbol] | | | 301 | | | | | [symbol] | [symbol] | [symbol] | 0 | 0 | [symbol] | [symbol] | [symbol] | [symbol] |
| [symbol] | | | [symbol] | | | | | [symbol] | [symbol] | 0 | 0 | [symbol] | [symbol] | [symbol] | [symbol] | [symbol] |
| [symbol] | | | [symbol] | | | | | 1373 | [symbol] | [symbol] | [symbol] | 21 | 1004 | [symbol] | [symbol] | [symbol] |
| [symbol] | | | [symbol] | | | | | 270 | [symbol] | 0 | [symbol] | 0 | [symbol] | [symbol] | 0 | [symbol] |
| [symbol] | | | [symbol] | | | | | 357 | 2017 | 0 | 304 | 0 | 0 | 1241 | [symbol] | [symbol] |
| [symbol] | | | [symbol] | | | | | 553 | 5137 | 0 | 0 | 0 | [symbol] | [symbol] | [symbol] | [symbol] |
| [symbol] | | | [symbol] | | | | | 0 | [symbol] | 0 | 21 | 0 | [symbol] | [symbol] | [symbol] | [symbol] |
| [symbol] | [symbol] | | | | | | | [symbol] | [symbol] | [symbol] | [symbol] | [symbol] | [symbol] | 1534 | 13700 | [symbol] |
| [symbol] | 161 | | | | | | | [symbol] | [symbol] | [symbol] | [symbol] | [symbol] | [symbol] | [symbol] | [symbol] | [symbol] |
| [symbol] | 150 | | | | | | | [symbol] | [symbol] | [symbol] | [symbol] | [symbol] | [symbol] | [symbol] | [symbol] | [symbol] |
| [symbol] | 157 | | | | | | | [symbol] | [symbol] | [symbol] | [symbol] | 1127 | [symbol] | [symbol] | [symbol] | [symbol] |
| hr5737 | 155 | | | | | | | 9 | [symbol] | 867 | [symbol] | 3447 | [symbol] | [symbol] | [symbol] | [symbol] |
| [symbol] | 153 | | | | | | | 2234 | [symbol] | [symbol] | [symbol] | [symbol] | [symbol] | [symbol] | [symbol] | [symbol] |
| MCF7 | 151 | | | | | | | [symbol] | [symbol] | [symbol] | [symbol] | [symbol] | [symbol] | [symbol] | [symbol] | [symbol] |
| [symbol] | [symbol] | | | | | | | 779 | [symbol] | 0 | 704 | 437 | 6271 | [symbol] | [symbol] | [symbol] |
| UACC257 | 147 | | | | | | | 169 | 14475 | 1430 | 740 | 183 | [symbol] | 2185 | 1031 | 2809 |
| UACC12 | 145 | | | | | | | [symbol] | [symbol] | $40 | 2105 | 4 | [symbol] | 1300 | 4231 | 41725 |
| EX142 | 144 | | | | | | | 40 | 12812 | 4444 | 577 | 779 | 8179 | 1030 | 2800 | 50031 |
| UC-31 | 143 | | | | | | | 445 | [symbol] | 1704 | [symbol] | [symbol] | 14004 | 2732 | 2034 | [symbol] |
| [symbol] | 142 | | | | | | | 1182 | 2782 | [symbol] | 7044 | 2900 | 13105 | 1215 | 9005 | 42911 |
| UCM-12 | 141 | | | | | | | 325 | 5737 | 2512 | 104 | 0 | [symbol] | 1317 | 64 | [symbol] |
| [symbol] | 140 | | | | | | | 1557 | 14400 | 2231 | 796 | 1827 | 7340 | 1575 | 7479 | 67511 |
| HCT-15 | 130 | | | | | | | 740 | 12275 | 771 | 687 | 996 | 4821 | 2007 | 7421 | 71185 |
| [symbol] | 134 | | | | | | | 1725 | 25043 | 2545 | 2391 | 2583 | 10072 | 2036 | 7221 | 42987 |
| COLO204 | 137 | | | | | | | 2434 | [symbol] | 2785 | 430 | 1949 | 13401 | 1001 | 7097 | [symbol] |
| LOX [symbol] | 134 | | | | | | | 203 | 37437 | 202 | 3161 | 4330 | 30430 | 402 | 1061 | [symbol] |
| [symbol] | 135 | | | | | | | [symbol] | 6511 | 0 | 1407 | [symbol] | [symbol] | [symbol] | [symbol] | [symbol] |
| TR [symbol] | 134 | | | | | | | 545 | 6427 | 1317 | 1524 | 1082 | [symbol] | 1711 | 11301 | 42230 |
| HCT115 | 133 | | | | | | | 1016 | 7308 | 0 | 140 | 413 | 11724 | 2913 | [symbol] | $4 |
| 794-4 | 132 | | | | | | | 636 | 8319 | 827 | 1162 | 747 | [symbol] | 1479 | 7153 | 41433 |
| HCC-2000 | 131 | | | | | | | 1700 | 2340 | 0 | [symbol] | 1115 | 3212 | 1584 | 3254 | 71444 |
| AC [symbol] | 130 | | | | | | | 600 | [symbol] | 1424 | 1233 | 405 | 21377 | 1408 | 7023 | 48774 |
| PC-3 | 129 | | | | | | | 577 | [symbol] | 0 | 33 | [symbol] | 14200 | 1430 | 3151 | 46670 |
| [symbol] | 128 | | | | | | | 1047 | 12023 | 1508 | 4327 | 377 | 29444 | [symbol] | 13036 | 43831 |
| [symbol] | 127 | | | | | | | 1200 | 1043 | 100 | 125 | 175 | [symbol] | 2170 | [symbol] | [symbol] |
| C [symbol] 1 | 126 | | | | | | | 201 | 13870 | [symbol] | [symbol] | 334 | [symbol] | 1833 | 15823 | 42579 |
| SA | 125 | | | | | | | 0 | [symbol] | 0 | [symbol] | 357 | 1134 | 2004 | 4030 | 44677 |
| [symbol] | 124 | | | | | | | 8313 | 19949 | 351 | 295 | 0 | 14125 | 2475 | 10 [symbol] | [symbol] |
| RPLA1225 | 123 | | | | | | | 0 | [symbol] | 0 | 1 | 112 | 590 | 2000 | 5704 | 63175 |
| 3N12C | 122 | | | | | | | 0 | 17323 | 0 | 2902 | 134 | 5734 | 2441 | 10677 | [symbol] |
| HL-90 | 121 | | | | | | | 303 | 4211 | 541 | 116 | 240 | 1354 | 2057 | 2399 | [symbol] |
| MCIT-4 | 120 | | | | | | | 226 | 4494 | 2178 | 551 | 111 | 5801 | 1313 | 7213 | 70180 |
| [symbol] | 119 | | | | | | | 854 | 10544 | 6273 | 2442 | 677 | 12121 | [symbol] | 23428 | 40594 |
| K-852 | 118 | | | | | | | 1044 | 48121 | 2214 | [symbol] | [symbol] | 5279 | 1771 | [symbol] | [symbol] |
| OVCAR-4 | 117 | | | | | | | 21340 | 13447 | 4368 | 240 | 3540 | 14315 | 1400 | 21304 | [symbol] |
| CCRF-CEM | 116 | | | | | | | [symbol] | 31473 | [symbol] | 4834 | [symbol] | 4841 | 1450 | 7736 | $4 |
| OVCAR-3 | 115 | | | | | | | 543 | [symbol] | 2364 | 1091 | 119 | 3448 | 1421 | 45004 | 42391 |
| SF-530 | 114 | | | | | | | 1113 | 7236 | 0 | [symbol] | 834 | 10017 | 1247 | 4954 | 41079 |
| HCR [symbol] | 113 | | | | | | | 0 | 6295 | 2042 | 6254 | 47 | 16794 | [symbol] | [symbol] | [symbol] |
| SF-296 | 112 | | | | | | | 209 | 7254 | 304 | 1051 | 2774 | 8751 | 675 | [symbol] | 47319 |
| AS [symbol] TCC | 111 | | | | | | | 1371 | 14734 | 0 | 3749 | 2418 | 18206 | 1203 | [symbol] | [symbol] |
| SF-282 | 110 | | | | | | | 1377 | 11909 | 0 | 2303 | 1054 | 18314 | 1000 | 8470 | $4 |
| NCH622 | 109 | | | | | | | 325 | 10129 | 1948 | 3305 | 1283 | 17942 | 3094 | 15205 | [symbol] |
| [symbol] | 108 | | | | | | | 1614 | 9800 | 2187 | 374 | 1011 | 2018 | 2229 | 1000 | [symbol] |
| [symbol] | 107 | | | | | | | [symbol] | 11864 | [symbol] | [symbol] | 830 | [symbol] | [symbol] | [symbol] | [symbol] |
| SNB-75 | 106 | | | | | | | 252 | 7303 | 127 | 623 | 25 | 20030 | 1048 | 1500 | 40300 |
| [symbol] | 105 | | | | | | | 2683 | 13987 | 3336 | 3617 | 473 | 11000 | [symbol] | [symbol] | [symbol] |
| [symbol] | 104 | | | | | | | 0 | 7540 | 1418 | [symbol] | 329 | [symbol] | [symbol] | 6129 | 52719 |
| HCHH226 | 103 | | | | | | | 823 | 19491 | 1502 | 2302 | 372 | 7883 | 4474 | 13435 | 103482 |
| [symbol] | 102 | | | | | | | 0 | [symbol] | 471 | 740 | 482 | 5154 | 2796 | 5302 | 55779 |
| HCH4427 | 101 | | | | | | | 872 | 12835 | 1794 | 6027 | 715 | 1791 | 3125 | 30347 | 54491 |
| [symbol] | 100 | | | | | | | 672 | 10001 | 3057 | 275 | 665 | 3254 | 3272 | 7799 | 54479 |
| EKVX | 99 | | | | | | | 0 | [symbol] | [symbol] | [symbol] | 217 | 4085 | 3078 | 31152 | 43479 |
| OVCAR-8 | 98 | | | | | | | 347 | 7231 | 2432 | 897 | 870 | 8534 | 1949 | 4820 | [symbol] |
| HOP-82 | 97 | | | | | | | 474 | 12057 | [symbol] | [symbol] | 782 | 12342 | 2345 | 31301 | [symbol] |
| [symbol] | 48 | | | | | | | 1806 | 4800 | 0 | 3634 | 315 | 34750 | 1914 | 5355 | [symbol] |
| [symbol] | 47 | | | | | | | 37 | 3434 | 0 | 477 | 80 | [symbol] | [symbol] | [symbol] | [symbol] |
| [symbol] | 46 | | | | | | | 4753 | 10427 | 0 | 2520 | 7034 | [symbol] | 1175 | 5191 | [symbol] |
| TOOP | 38 | | | | | | | 0 [symbol] | 13483 | 55 | 30915 | 701 | [symbol] | 2429 | 0414 | 11036 |
| ASAS-1 | | | | | | | [symbol] | 0 | 2004 | [symbol] | 13218 | 0 | 1876 | 331 | 14999 | 18283 |
| ASAS-3 | | | | | | | [symbol] | 0 | 2582 | 0 | 263 | 0 | 317 | 314 | 3467 | 10191 |
| ASAS-4 | | | | | | | [symbol] | 0 | 3700 | 0 | [symbol] | 0 | 573 | 117 | 11425 | 10082 |
| ASAS-5 | | | | | | | [symbol] | 0 | 4477 | 0 | 2173 | 0 | 400 | 242 | 15449 | 13282 |
| ASAS-7 | | | | | | | [symbol] | 0 | 3411 | 0 | 578 | 0 | 1883 | 291 | 12240 | 20201 |
| EKVX-1 | | | | | | | [symbol] | 0 | 7918 | 278 | 10025 | 0 | 2348 | 602 | 27454 | 14030 |
| EKVX-4 | | | | | | | [symbol] | 0 | 5808 | 0 | 7480 | 0 | 3883 | 1803 | 78164 | [symbol] |
| EKVX-3 | | | | | | | [symbol] | 0 | 4165 | 0 | 6533 | 0 | 1342 | 347 | 7116 | [symbol] |
| EKVX-8 | | | | | | | [symbol] | 0 | 2042 | 442 | 1804 | 0 | 2088 | 781 | 18042 | 27344 |
| EKVX-7 | | | | | | | [symbol] | 0 | [symbol] | 3404 | 40 | 0 | 1233 | 1394 | 1345 | [symbol] |
| MCF-7-1 | | | | | | | [symbol] | 0 | 2495 | 0 | 3485 | 0 | 386 | 267 | 11222 | 16811 |
| MCF-7-3 | | | | | | | [symbol] | 0 | 4324 | 0 | 7219 | 0 | 177 | 321 | 10076 | 14247 |
| MCF-7-4 | | | | | | | [symbol] | 0 | [symbol] | 862 | 237 | 0 | 707 | 191 | [symbol] | 19080 |
| MCF-7-5 | | | | | | | [symbol] | 0 | 4641 | 0 | 312 | 0 | 0 | 80 | 24357 | 14439 |
| MCF-7-7 | | | | | | | [symbol] | 0 | 2971 | 0 | 341 | 0 | 300 | 119 | 9134 | 14350 |
| ADA-RES-1 | | | | | | | [symbol] | 0 | 13125 | 0 | [symbol] | 0 | 1135 | [symbol] | 43482 | 43329 |
| ADA-RES-3 | | | | | | | [symbol] | 0 | [symbol] | 206 | [symbol] | 0 | 583 | 715 | 8379 | 15027 |
| ADA-RES-4 | | | | | | | [symbol] | 0 | [symbol] | 812 | 79 | 0 | 7847 | [symbol] | [symbol] | 22904 |
| ADA-RES-5 | | | | | | | [symbol] | 0 | [symbol] | 0 | [symbol] | 0 | 572 | 115 | [symbol] | [symbol] |
| ADA-RES-7 | | | | | | | [symbol] | 0 | [symbol] | 0 | [symbol] | 0 | 835 | 310 | [symbol] | 17945 |
| [symbol] -1 | | | | | | | [symbol] | 0 | [symbol] | 0 | 1743 | 0 | 490 | [symbol] | [symbol] | 25030 |
| [symbol] -3 | | | | | | | [symbol] | 0 | [symbol] | 317 | [symbol] | 0 | 2774 | 777 | 23794 | 13274 |
| [symbol] -4 | | | | | | | [symbol] | 0 | 8701 | 0 | [symbol] | 0 | [symbol] | 794 | 23304 | 14200 |
| [symbol] -5 | | | | | | | [symbol] | 0 | [symbol] | 0 | [symbol] | 0 | 1877 | 750 | [symbol] | 17103 |
| [symbol] -7 | | | | | | | [symbol] | 0 | 8101 | [symbol] | [symbol] | 0 | [symbol] | 1033 | [symbol] | 13302 |
| [symbol] -1 | | | | | | | [symbol] | 0 | 2972 | 1045 | 125 | 0 | 783 | 341 | [symbol] | 13205 |
| [symbol] -3 | | | | | | | [symbol] | 0 | 6135 | 209 | 2900 | 0 | [symbol] | 304 | 15037 | 12311 |
| [symbol] -4 | | | | | | | [symbol] | 0 | [symbol] | 787 | 1330 | 0 | [symbol] | 419 | [symbol] | 10755 |
| [symbol] -5 | | | | | | | [symbol] | 0 | 8774 | 343 | 2004 | 0 | 2129 | 834 | [symbol] | 18512 |
| [symbol] -7 | | | | | | | [symbol] | 0 | 8057 | 0 | 1004 | 0 | 1573 | 840 | [symbol] | 19167 |
| [symbol] -1 | | | | | | | [symbol] | 0 | 5732 | 0 | [symbol] | 0 | 400 | 783 | 13204 | 13627 |
| [symbol] -3 | | | | | | | [symbol] | 0 | [symbol] | 0 | [symbol] | 0 | [symbol] | 547 | [symbol] | 17407 |
| [symbol] -4 | | | | | | | [symbol] | 0 | 11579 | 0 | 2003 | 0 | [symbol] | 567 | [symbol] | 19523 |
| [symbol] -5 | | | | | | | [symbol] | 0 | [symbol] | 0 | 2000 | 0 | 1547 | 553 | [symbol] | 17747 |
| [symbol] -7 | | | | | | | [symbol] | 0 | [symbol] | 67 | 2047 | 0 | [symbol] | 534 | 13374 | 13714 |
| [symbol] -2 | | | | | | | [symbol] | 0 | 1774 | 0 | 7823 | 0 | [symbol] | 473 | 3747 | 14537 |
| [symbol] -2 | | | | | | | [symbol] | 0 | 4310 | 0 | [symbol] | 0 | [symbol] | 344 | 14174 | [symbol] |
| [symbol] -1 | | | | | | | [symbol] | 0 | [symbol] | 0 | 1131 | 0 | [symbol] | [symbol] | 13342 | 12401 |
| [symbol] -2 | | | | | | | [symbol] | 0 | 15450 | 0 | [symbol] | 0 | [symbol] | [symbol] | [symbol] | [symbol] |
| [symbol] -2 | | | | | | | [symbol] | 0 | 3414 | [symbol] | [symbol] | 0 | [symbol] | 541 | [symbol] | [symbol] |
| [symbol] -1 | | | | | | | [symbol] | 0 | [symbol] | 167 | 302 | 0 | 774 | 285 | [symbol] | 14833 |
| [symbol] -2 | | | | | | | [symbol] | 0 | [symbol] | 0 | 813 | 0 | 0 | 247 | [symbol] | 18521 |
| [symbol] - | | | | | | | [symbol] | 0 | [symbol] | 0 | 1104 | 0 | [symbol] | 1129 | [symbol] | 14395 |
| [symbol] -2 | | | | | | | [symbol] | 0 | 6717 | 0 | 10047 | 0 | 3007 | 1179 | [symbol] | 34251 |
| [symbol] 4-1 | | | | | | | [symbol] | 0 | [symbol] | 704 | 1307 | 0 | [symbol] | 878 | [symbol] | [symbol] |
| [symbol] 4-2 | | | | | | | [symbol] | 0 | [symbol] | [symbol] | 3777 | 0 | 1872 | 843 | [symbol] | 11007 |
| [symbol] 5-1 | | | | | | | [symbol] | 0 | 3130 | [symbol] | 334 | 0 | 851 | [symbol] | [symbol] | 11500 |
| [symbol] 5-2 | | | | | | | [symbol] | 0 | [symbol] | 0 | 829 | 0 | 344 | 270 | [symbol] | 11700 |
| [symbol] -2 | | | | | | | [symbol] | 0 | [symbol] | 281 | 4447 | 0 | [symbol] | 122 | [symbol] | [symbol] |
| [symbol] -2 | | | | | | | [symbol] | 0 | [symbol] | 0 | 2034 | 0 | 814 | 834 | [symbol] | [symbol] |
| [symbol] -2 | | | | | | | [symbol] | 0 | [symbol] | [symbol] | [symbol] | 0 | 4434 | 842 | 40773 | [symbol] |
| [symbol] -1 | | | | | | | [symbol] | 0 | [symbol] | 33 | 2311 | 0 | [symbol] | 1162 | [symbol] | [symbol] |
| [symbol] -2 | | | | | | | [symbol] | 0 | [symbol] | 0 | [symbol] | 0 | 915 | 534 | [symbol] | [symbol] |
| [symbol] -2 | | | | | | | [symbol] | 0 | [symbol] | 0 | [symbol] | 0 | 1503 | 844 | [symbol] | 34461 |
| [symbol] -1 | | | | | | | [symbol] | 0 | [symbol] | 0 | 0 | 0 | 570 | 230 | [symbol] | 12314 |
| [symbol] -2 | | | | | | | [symbol] | 0 | [symbol] | 404 | 429 | 0 | 970 | 200 | [symbol] | [symbol] |
| [symbol] -1 | | | | | | | [symbol] | 0 | [symbol] | 0 | [symbol] | 0 | 2310 | [symbol] | [symbol] | 31144 |
| [symbol] -2 | | | | | | | [symbol] | 0 | [symbol] | 743 | 8373 | 0 | [symbol] | 1031 | [symbol] | 27447 |
| [symbol] -1 | | | | | | | [symbol] | 0 | 7177 | 447 | [symbol] | 0 | 1155 | 415 | 11483 | [symbol] |
| [symbol] -2 | | | | | | | [symbol] | 0 | [symbol] | 403 | 2004 | 0 | 3334 | 745 | [symbol] | 17007 |
| [symbol] -2 | | | | | | | [symbol] | 0 | [symbol] | [symbol] | 1083 | 0 | [symbol] | 732 | [symbol] | 13250 |
| [symbol] -1 | | | | | | | | 0 | [symbol] | 0 | 2714 | 0 | 1113 | 300 | [symbol] | 14813 |
| [symbol] -2 | | | | | | | | 0 | 12729 | 0 | 126 | 0 | [symbol] | 300 | [symbol] | [symbol] |
| [symbol] -3 | | | | | | | | 0 | [symbol] | 0 | 354 | 0 | [symbol] | 400 | 44133 | [symbol] |
| [symbol] -4 | | | | | | | | 0 | [symbol] | 0 | 0 | 0 | [symbol] | 500 | 4312 | 22074 |
| [symbol] -5 | | | | | | | | 0 | 2003 | 0 | 0 | 0 | 0 | 404 | 0 | [symbol] |
| [symbol] | | | | | | | | 0 | 13424 | 0 | 0 | 0 | 3344 | 1234 | [symbol] | [symbol] |
| [symbol] | | | | | | | | 0 | 15247 | 0 | 1442 | 0 | 3704 | [symbol] | [symbol] | [symbol] |
| [symbol] | | | | | | | | 0 | 12139 | 0 | 309 | 0 | 2077 | 1999 | [symbol] | [symbol] |
| D [symbol] 7 | | | | | | | | 0 | [symbol] 7 [symbol] | 3124 | 2233 | 0 | 2724 | 1021 | 10438 | 11243 |
| D [symbol] -4 | | | | | | | | 0 | 33351 | 84 | 33 [symbol] | 0 | 3479 | [symbol] | 2370 | 14 [symbol] 61 |
| D [symbol] -5 | | | | | | | | 0 | 22317 | 0 | 4238 | 0 | 4247 | 704 | 52752 | 12234 |
| D [symbol] -11 | | | | | | | | 0 | 42 [symbol] | 0 | 4722 | 0 | 327 [symbol] | 1044 | 48334 | 73174 |
| D [symbol] -12 | | | | | | | | 0 | 14274 | 0 | 1457 | 0 | 2734 | [symbol] | 60043 | 11302 |
| D [symbol] -10 | | | | | | | | 0 | 1 [symbol] 15 | 0 | 13 [symbol] | 0 | 4270 | 841 | 34042 | 11401 |
| D [symbol] -1 | | | | | | | | 0 | 11734 | 0 | 21443 | 0 | 3074 | 1022 | 20301 | 22 [symbol] |
| D [symbol] -2 | | | | | | | | 0 | 1337 | 0 | 13705 | 0 | 1120 | 783 | 15242 | 14134 |
| D [symbol] -3 | | | | | | | | 0 | [symbol] 5 | 1171 | [symbol] 13 | 0 | 103 | 123 | 31807 | 2 [symbol] |
| D [symbol] -4 | | | | | | | | 0 | [symbol] | 0 | 550 | 0 | 1704 | 480 | 30143 | 23124 |
| D [symbol] -5 | | | | | | | | 0 | 3723 | 0 | 215 | 0 | [symbol] | 6 [symbol] | 1831 | 12344 |
| D [symbol] -6 | | | | | | | | 0 | [symbol] | 0 | 0 | 0 | 485 | 452 | 1823 | [symbol] |
| A [symbol] -4 | | | | | | | wt | 0 | 4318 | 537 | 1575 | 0 | 2240 | 304 | 18012 | 13423 |
| EXVX-4 | | | | | | | [symbol] | 0 | 4743 | 0 | 4381 | 0 | 4341 | 102 | 24283 | 14 [symbol] |
| HCT-114-7 | | | | | | | wt | 0 | 2845 | 507 | [symbol] | 0 | 512 | 478 | 8413 | 12443 |
| HCT-114-8 | | | | | | | wt | 0 | 2144 | 3418 | [symbol] | 0 | 382 | 381 | 11432 | 14 [symbol] 04 |
| HT29-1 | | | | | | | [symbol] | 0 | 2414 | 0 | 1343 | 0 | 1404 | 431 | 23418 | 21731 |
| HT29-7 | | | | | | | [symbol] | 0 | 1283 | 0 | 64 | 0 | 307 | 375 | 5185 | 13142 |
| HT29-8 | | | | | | | [symbol] | 0 | 4333 | 0 | 2552 | 0 | 702 | [symbol] | 14207 | [symbol] |
| 4F [symbol] -7 | | | | | | | wt | 0 | [symbol] 02 | 0 | [symbol] | 0 | 1074 | 1025 | 12404 | 17428 |
| 3F [symbol] -8 | | | | | | | wt | 0 | [symbol] 54 | 0 | 130 | 0 | 544 | 011 | 14374 | [symbol] |
| 3F [symbol] -7 | | | | | | | [symbol] | 0 | [symbol] 10 | 14 [symbol] | 708 | 0 | 1316 | 420 | 14244 | 15443 |
| 3F [symbol] -8 | | | | | | | [symbol] | 0 | 8205 | 0 | 709 | 0 | [symbol] | 811 | [symbol] | [symbol] |
| OVCAR-4-7 | | | | | | | wt | 0 | 1744 | 0 | [symbol] | 0 | [symbol] | 420 | [symbol] | [symbol] |
| OVCAR-4-8 | | | | | | | wt | 0 | 4200 | [symbol] | 307 | 0 | [symbol] | [symbol] | [symbol] | [symbol] |
| OVCAR-4-7 | | | | | | | [symbol] | 0 | 4231 | 0 | 12121 | 0 | [symbol] | 730 | [symbol] | [symbol] |
| OVCAR-5-8 | | | | | | | [symbol] | 0 | 3470 | 17044 | 1790 | 0 | [symbol] | 1330 | [symbol] | [symbol] |
| [symbol] | | | | | | | wt | 0 | 4347 | 348 | 48 | 0 | [symbol] | [symbol] | [symbol] | [symbol] |
| AOR- [symbol] -4 | | | | | | | [symbol] | 0 | 4033 | 485 | 2805 | 0 | [symbol] | 451 | [symbol] | [symbol] |
| [symbol] -8 | | | | | | | [symbol] | 0 | 4132 | 914 | 1079 | 0 | [symbol] | [symbol] | [symbol] | [symbol] |
| [symbol] -7 | | | | | | | [symbol] | 0 | 3841 | 850 | [symbol] | 0 | [symbol] | 704 | [symbol] | [symbol] |
| SW480-8 | | | | | | | [symbol] | 0 | 3005 | [symbol] | 1133 | 0 | [symbol] | 212 | [symbol] | [symbol] |
| SW480-9 | | | | | | | [symbol] | 0 | 5441 | 8374 | 1442 | 0 | [symbol] | 450 | [symbol] | [symbol] |
| C33A-7 | | | | | | | [symbol] | 0 | 4445 | 0 | 131 | 0 | [symbol] | [symbol] | [symbol] | [symbol] |
| C33A-8 | | | | | | | [symbol] | 0 | 4384 | 0 | 853 | 0 | [symbol] | 231 | [symbol] | [symbol] |
| C33A-7 | | | | | | | [symbol] | 0 | [symbol] 43 | [symbol] | 1821 | 0 | [symbol] | 1472 | [symbol] | [symbol] |
| U2O5-8 | | | | | | | [symbol] | 0 | 4223 | 187 | 482 | 0 | [symbol] | [symbol] | [symbol] | [symbol] |
| U2O5-7 | | | | | | | wt | 0 | 4235 | 17844 | 1101 | 0 | [symbol] | 614 | [symbol] | [symbol] |
| H [symbol] -8 | | | | | | | wt | 0 | 4438 | 0 | 0 | 0 | [symbol] | 614 | [symbol] | 18314 |
| H [symbol] -8 | | | | | | | wt | 0 | [symbol] | 0 | [symbol] | 322 | [symbol] | [symbol] | [symbol] | 17704 |
| 414 [symbol] | | | | | | | | 218 | 10478 | 78 | 471 | [symbol] | [symbol] | [symbol] | 4323 | [symbol] |
| CR 1572 317 | | | | | | | | 0 | 1424 | 0 | 182 | 238 | [symbol] | [symbol] | [symbol] | [symbol] |
| [symbol] | | | | | | 54 | | 1350 | 7174 | 308 | 48 | 43 | [symbol] | [symbol] | 13124 | [symbol] |
| HT348 | | | | | | | | 415 | 10973 | 0 | 187 | 825 | 0 | 2041 | [symbol] | [symbol] |
| HT370 | | | | | | | | 244 | 1570 | 1052 | 0 | 147 | 4171 | [symbol] | [symbol] | [symbol] |
| HT385 | | | | | | | | 2 [symbol] 4 | 23774 | 0 | 68 | 40 | 1211 | 1344 | 2271 | [symbol] |
| HT [symbol] | | | | | | | | 484 | 4382 | 0 | [symbol] | 0 | 870 | [symbol] | [symbol] | [symbol] |
| [symbol] -3 | | | | | | 173 | | 142 | 2823 | 0 | [symbol] | 0 | 1075 | [symbol] | 2813 | [symbol] |
| [symbol] -4 | | | | | | 175 | | 1224 | 107 | 815 | 0 | 878 | 1984 | [symbol] | [symbol] | [symbol] |
| [symbol] -5 | | | | | | 177 | | 0 | 1832 | 0 | 95 | 31 | 1453 | [symbol] | 783 | 37803 |
| [symbol] | | | | | | | | 4 | 8302 | 811 | 827 | 0 | 83 | [symbol] | 280 | 44307 |
| [symbol] | | | | | | 237 | | 1100 | 23331 | [symbol] 0 | 30 | 0 | 3 | [symbol] | [symbol] | [symbol] |
| MTB10 | | | | | | | | 0 | [symbol] 54 | [symbol] | 157 | 47 | 1031 | 8317 | 797 | 84103 |
| [symbol] 33142 [symbol] 2 | | | | | | | | 144 | 4732 | [symbol] | 300 | [symbol] | 1233 | [symbol] | [symbol] | [symbol] |
| [symbol] | | | | | | | | 87 | 100 | 0 | 191 | 1874 | 504 | [symbol] | 0 | [symbol] |
| [symbol] -OS [symbol] | | | | | | | | 0 | 4870 | 0 | [symbol] | 1131 | 1543 | [symbol] | [symbol] | [symbol] |
| 6A-OS [symbol] A | | | | | | | | [symbol] | 31232 | 830 | [symbol] | 0 | 840 | [symbol] | [symbol] | [symbol] |
| [symbol] A | | | | | | | | 272 | 20543 | 177 | [symbol] | 0 | 2913 | [symbol] | [symbol] | [symbol] |
| HCT-114-3 | | | | | | | wt | 0 | 2244 | [symbol] | 724 | 0 | 4200 | 247 | [symbol] | [symbol] |
| HCT-114-4 | | | | | | | wt | 0 | 347 | 1378 | 724 | 0 | [symbol] | [symbol] | [symbol] | [symbol] |
| HCT-114-5 | | | | | | | wt | 0 | 5154 | 0 | 2977 | 0 | [symbol] | [symbol] | [symbol] | [symbol] |
| HCT-114-6 | | | | | | | wt | 0 | 777 | 1117 | 42 | 0 | 477 | 315 | [symbol] | [symbol] |
| A34-8 | | | | | | | wt | 0 | 3371 | 80 | 7344 | 0 | [symbol] | 311 | [symbol] | [symbol] |
| HT29-3 | | | | | | | [symbol] | 0 | 879 | 1434 | 0 | 0 | [symbol] | [symbol] | [symbol] | [symbol] |
| EXVX-6 | | | | | | | [symbol] | 0 | 771 | 1300 | 875 | 0 | [symbol] | [symbol] | [symbol] | [symbol] |
| HT29-4 | | | | | | | [symbol] | 0 | 3428 | 811 | 4091 | 0 | [symbol] | 1083 | [symbol] | [symbol] |
| HT29-5 | | | | | | | [symbol] | 0 | 3134 | 1824 | 2404 | 0 | [symbol] | [symbol] | [symbol] | 27134 |
| HT29-6 | | | | | | | [symbol] | 0 | [symbol] | 0 | 4734 | 0 | 1303 | 230 | 28341 | [symbol] |
| OVCAR-3 | | | | | | | wt | 0 | [symbol] | 1901 | 1104 | 0 | 143 | [symbol] | [symbol] | [symbol] |
| OVCAR-4 | | | | | | | wt | 0 | [symbol] | [symbol] | 4205 | 0 | [symbol] | 373 | [symbol] | [symbol] |
| OVCAR-5 | | | | | | | wt | 0 | 3487 | 177 | 0 | 0 | [symbol] | 64 | [symbol] | 14135 |
| OVCAR-6 | | | | | | | wt | 0 | 12820 | 436 | 704 | 0 | 7830 | [symbol] | [symbol] | 15744 |
| [symbol] -3 | | | | | | | wt | 0 | [symbol] | [symbol] | 294 | 0 | 829 | [symbol] | [symbol] | [symbol] |
| [symbol] -4 | | | | | | | wt | 0 | [symbol] | 0 | 459 | 0 | 413 | 332 | [symbol] | 10033 |
| [symbol] -5 | | | | | | | wt | 0 | 11231 | [symbol] | 0 | 0 | 642 | [symbol] | [symbol] | [symbol] |
| [symbol] -6 | | | | | | | wt | 0 | 12348 | 0 | 1341 | 0 | 2321 | 27 | 14823 | 20785 |
| OVCAR-3 | | | | | | | [symbol] | 0 | 5756 | 0 | 1981 | 0 | 1507 | [symbol] | [symbol] | 14804 |
| OVCAR-4 | | | | | | | [symbol] | 0 | 4324 | 326 | [symbol] | 0 | [symbol] | [symbol] | 8731 | [symbol] |
| OVCAR-5 | | | | | | | [symbol] | 0 | [symbol] | 0 | 1852 | 0 | [symbol] | [symbol] | 310 | 11271 |
| OVCAR-6 | | | | | | | [symbol] | 0 | [symbol] | 0 | 322 | 0 | [symbol] | 141 | [symbol] | [symbol] |
| [symbol] | | | | | | | wt | 0 | 3371 | 70 | 2138 | 0 | 0 | [symbol] | [symbol] | 19834 |
| [symbol] | | | | | | | [symbol] | 0 | [symbol] | 0 | 10 | 0 | 1130 | [symbol] | [symbol] | [symbol] |
| [symbol] | | | | | | | [symbol] | 0 | [symbol] | 0 | [symbol] | 0 | 1108 | [symbol] | [symbol] | [symbol] |
| [symbol] -3 | | | | | | | [symbol] | 0 | 1457 | [symbol] | [symbol] | 0 | 393 | [symbol] | [symbol] | [symbol] |
| [symbol] -4 | | | | | | | [symbol] | 0 | 1242 | 0 | [symbol] | 0 | 481 | [symbol] | [symbol] | [symbol] |
| [symbol] -5 | | | | | | | [symbol] | 0 | 4786 | 184 | 1304 | 0 | 1516 | [symbol] | 37823 | 14105 |
| [symbol] -6 | | | | | | | [symbol] | 0 | 7887 | 0 | 484 | 0 | 0 | 173 | 30247 | 17087 |
| C33A-3 | | | | | | | [symbol] | 0 | 4159 | 0 | 831 | 0 | [symbol] | [symbol] | [symbol] | 7717 |
| C33A-4 | | | | | | | [symbol] | 0 | 4870 | [symbol] | [symbol] | 0 | [symbol] | [symbol] | 11378 | [symbol] |
| C33A-5 | | | | | | | [symbol] | 0 | 2983 | [symbol] | 0 | 0 | 1129 | [symbol] | [symbol] | [symbol] |
| C33A-6 | | | | | | | [symbol] | 0 | 4237 | 4219 | 344 | 0 | 457 | [symbol] | [symbol] | [symbol] |
| [symbol] | | | | | | | wt | 0 | 1801 | 0 | [symbol] | 0 | [symbol] | 473 | [symbol] | [symbol] |
| [symbol] -3 | | | | | | | [symbol] | 0 | [symbol] | 837 | [symbol] | 0 | 1449 | [symbol] | [symbol] | [symbol] |
| [symbol] -4 | | | | | | | [symbol] | 0 | 4249 | 10235 | $ | 0 | [symbol] | 475 | [symbol] | 23270 |
| [symbol] -5 | | | | | | | [symbol] | 0 | [symbol] | 1853 | 0 | 0 | [symbol] | [symbol] | [symbol] | 18157 |
| [symbol] -6 | | | | | | | [symbol] | 0 | 9431 | 0 | [symbol] | 0 | [symbol] | 540 | [symbol] | [symbol] |
| [symbol] -6 | | | | | | | wt | 0 | 10377 | 780 | 1233 | 0 | [symbol] | 340 | [symbol] | [symbol] |
| [symbol] -3 | | | | | | | wt | 0 | 17277 | 0 | 3974 | 0 | 3412 | 1982 | [symbol] | [symbol] |
| [symbol] -4 | | | | | | | wt | 0 | [symbol] | 0 | 23072 | 0 | 3413 | [symbol] | [symbol] | [symbol] |
| [symbol] -3 | | | | | | | [symbol] | 0 | [symbol] | 0 | 11184 | 0 | 1910 | [symbol] | [symbol] | 18347 |
| [symbol] -4 | | | | | | | [symbol] | 0 | 13125 | 0 | [symbol] | 0 | 1248 | 1137 | [symbol] | [symbol] |
| [symbol] -5 | | | | | | | [symbol] | 0 | 4084 | 0 | 0 | 0 | 814 | [symbol] | 2400 | [symbol] |
| [symbol] -6 | | | | | | | [symbol] | 0 | [symbol] | 0 | [symbol] | 0 | 318 | [symbol] | 32104 | [symbol] |
| [symbol] -13 | | | | | | | | 0 | [symbol] | 0 | [symbol] | 0 | [symbol] | 740 | [symbol] | 20303 |
| [symbol] -20 | | | | | | | | 0 | 14080 | 0 | [symbol] | 0 | 2419 [symbol] | 777 | 18057 | [symbol] |
| [symbol] 21 | | | | | | | | 0 | 13873 | 0 | 3178 | 0 | [symbol] | [symbol] | [symbol] | [symbol] |
| [symbol] 22 | | | | | | | | 0 | [symbol] | 0 | [symbol] | 0 | [symbol] | [symbol] | [symbol] | 18178 |
| OVCAR4-5 | | | | | | | [symbol] | 0 | 4712 | 0 | [symbol] | 0 | [symbol] | [symbol] | [symbol] | 7830 |
| [symbol] -10 | | | | | | | | 0 | 4712 | 0 | 3078 | | [symbol] 34 | [symbol] 7 | 1837 | [symbol] |
| [symbol] -11 | | | | | | | | 0 | 8270 | 0 | 4705 | 0 | [symbol] | [symbol] | [symbol] | 4271 |
| [symbol] -12 | | | | | | | | 0 | 10544 | 0 | [symbol] | 0 | 2390 | [symbol] | 42037 | [symbol] |
| [symbol] -13 | | | | | | | | 0 | [symbol] | 0 | [symbol] | 0 | 1883 | 810 | 43027 | [symbol] |
| [symbol] -14 | | | | | | | | 0 | 18318 | 0 | [symbol] | 0 | 432 | 1228 | [symbol] | 17823 |
| [symbol] -15 | | | | | | | | 0 | 70792 | 0 | [symbol] | 0 | 834 | [symbol] | [symbol] | 13732 |
| [symbol] -16 | | | | | | | | 0 | 7517 | 0 | 54 | 0 | 1777 | 710 | 18220 | 12001 |
| [symbol] -17 | | | | | | | | 0 | 9720 | 0 | [symbol] | 0 | 1273 | 874 | [symbol] | [symbol] |
| [symbol] -18 | | | | | | | | 0 | [symbol] | 0 | 734 | 0 | 2844 | 1438 | [symbol] | 21852 |
| [symbol] -19 | | | | | | | | 0 | [symbol] | 0 | 0 | 0 | 1173 | 649 | 22782 | 14100 |
| adrenal gland- [symbol] | | 1 | | | | | | 0 | 238 | 14723 | 24 [symbol] 7 | [symbol] 724 | 3477 | 13 [symbol] | 2 [symbol] | 13731 |
| lymph node- [symbol] | | 2 | | | | | | 0 | [symbol] | 22295 | 2 [symbol] 336 | [symbol] | [symbol] 22 | [symbol] 8 | 281 | 22 [symbol] |
| [symbol] - [symbol] | | 3 | | | | | | 0 | [symbol] 7 | [symbol] 34 | 2 [symbol] | [symbol] | [symbol] 724 | 447 | [symbol] | 12 [symbol] 3 |
| [symbol] gland- [symbol] | | 4 | | | | | | 0 | 0 | 4322 | 2741 | 3 [symbol] 3 | 30 | 24 | 100 | 2044 |
| [symbol] | | 5 | | | | | | 0 | 0 | 114 [symbol] | 2076 | 13 [symbol] | 8167 | 3530 | 51 | 1 [symbol] 108 |
| [symbol] - [symbol] | | 6 | | | | | | 0 | 530 | [symbol] | 17022 | [symbol] 24 | 2091 | 1052 | 241 | 115 [symbol] |
| [symbol] - [symbol] | | 7 | | | | | | 0 | 234 | 1244 [symbol] | [symbol] 1638 | [symbol] 237 | [symbol] 28 | 4341 | [symbol] | 211 [symbol] |
| [symbol] gland- [symbol] | | 8 | | | | | | 127 | 162 | [symbol] 23 | [symbol] 271 [symbol] | [symbol] 3 | [symbol] 067 | 4 [symbol] 1 | 23 | 11637 |
| [symbol] - [symbol] | | 9 | | | | | | 171 | 364 | 10 [symbol] 39 | 23 [symbol] 41 | [symbol] 24 [symbol] 5 | [symbol] 45 | 91 [symbol] | 55 | 23 [symbol] |
| [symbol] - [symbol] | | 10 | | | | | | 317 | 3 [symbol] | 10004 | 742 [symbol] 4 | [symbol] 7 [symbol] 7 | 63 [symbol] | 338 | 301 | [symbol] 01 |
| [symbol] - [symbol] | | 11 | | | | | | 254 | 634 | 2011 [symbol] | 241 [symbol] | [symbol] 75 [symbol] | [symbol] 311 | [symbol] 3 | 234 | [symbol] 38 |
| [symbol] - [symbol] | | 12 | | | | | | 51 | [symbol] | 11313 | [symbol] 47 | 232 [symbol] 4 | 21 [symbol] | 13 [symbol] | 74 | 11 [symbol] 7 |
| [symbol] - [symbol] | | 13 | | | | | | 434 | 207 | 16229 | [symbol] | 11634 | 10 [symbol] 41 | 435 | 2 [symbol] 4 | [symbol] |
| [symbol] - [symbol] | | 14 | | | | | | 0 | 523 | 13 [symbol] 40 | 77 [symbol] | 1022 [symbol] | 6 [symbol] 3 | 2034 | 7 [symbol] | [symbol] 23 |
| [symbol] - [symbol] | | 15 | | | | | | 0 | [symbol] | 277 [symbol] | 1 [symbol] 719 | 15 [symbol] | [symbol] | [symbol] 75 | 177 | 13041 |
| [symbol] - [symbol] | | 16 | | | | | | 791 | 1 [symbol] | 154 [symbol] | 110 [symbol] | 194712 | [symbol] 38 | 22 [symbol] 13 | [symbol] 4 | 3341 |
| [symbol] - [symbol] | | 17 | | | | | | 2 [symbol] | 1054 | 11 [symbol] 0 [symbol] | [symbol] | [symbol] 2 [symbol] 7 | 229 [symbol] | [symbol] 7 [symbol] 1 | 0 | 4 [symbol] 1 [symbol] |
| [symbol] - [symbol] | | 18 | | | | | | 230 | 2 [symbol] | 25312 | 141 [symbol] | [symbol] 40 | 23 [symbol] | 132 [symbol] | 4 [symbol] | 757 [symbol] |
| [symbol] - [symbol] | | 19 | | | | | | 218 | [symbol] | [symbol] 142 | 18 [symbol] | 91241 | 3 [symbol] | 3 [symbol] | 0 | [symbol] 4 [symbol] |
| [symbol] - [symbol] | | 20 | | | | | | [symbol] 5 | 340 | 779 [symbol] | 11044 | 84347 | 7 [symbol] | [symbol] 7 [symbol] | 41 | 3 [symbol] 3 |
| [symbol] - [symbol] | | 21 | | | | | | 372 | 254 | [symbol] 34 | 13 [symbol] 31 | 1 [symbol] 77 | 1734 | 354 | 100 | 3 [symbol] |
| [symbol] - [symbol] | | 22 | | | | | | 79 | 2 [symbol] | [symbol] 2 | 14 [symbol] | 95 [symbol] 0 | 22 [symbol] 4 | 345 | 233 | [symbol] 0 [symbol] 3 |
| [symbol] - [symbol] | | 23 | | | | | | 179 | 542 | 16 [symbol] | 13 [symbol] 22 | [symbol] 1 [symbol] | 2203 | 337 | 143 | 5791 |
| [symbol] - [symbol] | | 24 | | | | | | 0 | 2 [symbol] | 12342 | 12134 | 71122 | [symbol] 0 [symbol] | 1227 | [symbol] | 3742 |
| [symbol] - [symbol] | | 25 | | | | | | 0 | 373 | 21347 | 23 [symbol] 43 | 1 [symbol] 40 | [symbol] | [symbol] 35 | [symbol] 2 | 2 [symbol] 00 |
| [symbol] - [symbol] | | 27 | | | | | | 177 | 496 | 21278 | 27103 | 112750 | 3483 | 700 | 290 | 3 [symbol] 7 [symbol] |
| [symbol] | | 28 | | | | 28 | | 0 | 124 | 6795 | 13179 | [symbol] 764 | 209 | [symbol] 39 | 2 [symbol] | 131 [symbol] |
| [symbol] gland- [symbol] | | 29 | | | | | | 0 | 229 | 13 [symbol] 48 | [symbol] | 11034 [symbol] | 72 [symbol] 0 | 14 [symbol] | [symbol] 2 | [symbol] 34 |
| [symbol] C | | 30 | | | | 30 | | 122 | 0 | 0 | 4 [symbol] 1 | 1157 [symbol] 0 | 11 | 372 | 7 | [symbol] 48 |
| [symbol] - [symbol] | | 31 | | | | | | 0 | 438 | 19832 | 13704 | [symbol] 718 | 2751 | 147 [symbol] | 162 | 11043 |
| [symbol] C | | 32 | | | | | | 0 | 797 | 167 [symbol] 4 | [symbol] 112 | 15 [symbol] 44 | 1037 | 304 | 471 | 20117 |
| [symbol] - [symbol] | | 33 | | | | | | 105 | 1371 | 61570 | 11723 | 84250 | 3429 | 1 [symbol] 4 | 81 | 18387 |
| HCA [symbol] C | | 34 | | | | | | 30 | 0 | 11 [symbol] 02 | 3716 | 222 [symbol] 4 | 200 | 230 | 0 | [symbol] 100 |
| [symbol] - [symbol] | | 35 | | | | | | 0 | 130 | 20403 | 2248 | 127701 | 2304 | 1034 | 0 | 7344 |
| lymph node | | 36 | | | | | | 0 | 154 | 2145 | 4941 | 17824 | [symbol] 76 | 0 | 54 | 2943 |
| [symbol] - [symbol] | | 37 | | | | | | 0 | 0 | 23 [symbol] 6 | 1 [symbol] 79 | 157218 | 302 | 613 | 0 | 3016 |
| [symbol] - [symbol] | | 38 | | | | | | 436 | 37 | 37 | 63 | [symbol] | 411 | 8 | 0 | 05 |
| Heart | | 39 | | | | | | 124 | 232 | 20524 | 4904 | 128875 | [symbol] | 23 | 233 | 4434 |
| [symbol] , [symbol] | | 40 | | | | | | 362 | 0 | 3423 | 14731 | 15 [symbol] | 423 | 184 | 0 | 4404 |
| C [symbol] - [symbol] | | 41 | | | | | | 0 | 0 | 0 | 4472 | 83230 | 76 | [symbol] 7 | [symbol] 4 | 82 [symbol] 3 |
| [symbol] - [symbol] | | 42 | | | | | | 534 | 204 | 1307 | 19012 | 1837 | 1024 | 330 | 2 [symbol] 0 | 7734 |
| Sal [symbol] - [symbol] | | 43 | | | | | | 4 | 170 | 4203 | [symbol] 25 | 1 [symbol] 0729 | 1547 | [symbol] 4 | 167 | 33 [symbol] |
| lymph- [symbol] | | 44 | | | | | | 441 | 218 | [symbol] 77 | 17243 | 23307 | 40 [symbol] 4 | 37 [symbol] | 304 | [symbol] 257 |
| HT21- [symbol] | | | | | 3 [symbol] | | | 154 | 34 | 0 | 1327 | 1011 | 0 | 384 | 40 | 2473 |
| HT21- [symbol] | | | | | 3 [symbol] | | | 0 | 0 | 0 | 1256 | 0 | 0 | 308 | [symbol] 2 | 5 [symbol] 3 |
| HT157- [symbol] | | | | | 3 [symbol] 1 | | | 44 | 0 | 0 | 3044 | 0 | 234 | 15 | 67 | 4 [symbol] 04 |
| [symbol] -13 | | | | | 364 | 356 | | 2341 | 0 | 0 | 4870 | 3 [symbol] 18 | 47 | 1438 | 140 | [symbol] 5 |
| [symbol] -12 | | | | | 364 | 354 | | 4181 | 44 | 1 [symbol] | [symbol] 004 | 3 [symbol] 3 | 530 | 475 | 243 | 1 [symbol] 145 |
| [symbol] | | | | | 344 | | | 76 | 41 | 0 | 3534 | 0 | 418 | 80 | 0 | 2922 |
| [symbol] | | | | | 342 | | | 258 | 148 | 230 | 2481 | 0 | 228 | 97 | 0 | 2043 |
| RFTEC | | | | | 334 | 334 | | 290 | 0 | 0 | 8453 | 4946 | 0 | 423 | 0 | 2443 |
| lymph node- [symbol] | | | | | 332 | | | 15 | 0 | 0 | 19034 | 0 | 104 | 92 | 0 | 564 |
| [symbol] SACC [symbol] - [symbol] | | | | | 330 | | | 0 | 0 | 823 | 6104 | 0 | 129 | 0 | 0 | 1081 |
| [symbol] - [symbol] | | | | | 329 | | | [symbol] | 0 | [symbol] 031 | 2 [symbol] | 43113 | 442 | [symbol] | 147 | 7317 |
| HT [symbol] - [symbol] | | | | | 327 | | | [symbol] | 0 | 272 | 2277 | 0 | 0 | 114 | 148 | 2 [symbol] 2 |
| [symbol] , [symbol] | | | | | 326 | | | 2058 | 25 | 417 | 11256 | 201 [symbol] | 125 | 871 | 0 | [symbol] |
| HT [symbol] - [symbol] | | | | | 321 | | | 9 | 20 | 0 | 2 [symbol] 34 | 0 | 0 | 24 [symbol] | 0 | 2382 |
| [symbol] 3rd [symbol] | | | | | 320 | | | 253 | 0 | [symbol] | [symbol] 611 | 77802 | 1049 | 307 | 294 | 1 [symbol] 0 |
| [symbol] - [symbol] | | | | | 319 | | | 447 | 3490 | 5764 | 177 [symbol] | 110677 | 52 [symbol] | 9031 | 12 [symbol] | 132 [symbol] 1 |
| [symbol] - [symbol] | | | | | 315 | | | 227 | 290 | 29285 | [symbol] 9 | 19054 | 2424 | [symbol] 3 | 79 | 9214 |
| [symbol] - [symbol] | | | | | 314 | | | 230 | 470 | 20 [symbol] | 12 [symbol] | 4 [symbol] 2 | 1 [symbol] 3 | 1002 | 273 | 14207 |
| [symbol] - [symbol] | | | | | 311 | | | 90 | 52 | 42 | 73 [symbol] | 314 | 173 | 9 | 162 | 1713 |
| [symbol] - [symbol] | | | | | 309 | | | 0 | 21 | 308 | 2 [symbol] 37 | 0 | 145 | 49 | 0 | 1645 |
| [symbol] gland | | | | | 307 | | | 24 [symbol] | 0 | 225 | 54 [symbol] | 19 | 169 | 57 [symbol] | 190 | 144 [symbol] |
| [symbol] - [symbol] | | | | | 304 | | | 0 | 11 | 194 | 4771 | 847 | [symbol] 36 | 19 | 0 | 2 [symbol] 9 |
| [symbol] gland | | | | | 303 | | | 146 | 734 | 3409 | 7475 | 735 | 1902 | 150 | 34 | 9057 |
| [symbol] - [symbol] | | | | | [symbol] | | | 0 | [symbol] | 27084 | 14441 | 141733 | 106 | 400 | 10 | 3863 |
| [symbol] - [symbol] | | | | | 296 | | | [symbol] | 1134 | 7043 | [symbol] | 9706 | 3876 | 2877 | [symbol] | 19783 |
| [symbol] - [symbol] | | | | | [symbol] | | | [symbol] | 344 | 14347 | [symbol] | 12364 | 1179 | 1089 | 384 | [symbol] |
| [symbol] - [symbol] | | | | | 296 | | | 0 | 344 | 702 | [symbol] | [symbol] | 2347 | 132 | 153 | 7683 |
| [symbol] - [symbol] | | | | | 294 | | | 485 | [symbol] | 546 | 4105 | 13000 | 4373 | [symbol] | 311 | 11648 |
| [symbol] - [symbol] | | | | | 292 | | | 136 | 84 | 1830 | 4123 | 279 | 479 | 231 | 134 | 5612 |
| [symbol] - [symbol] | | | | | 230 | | | 833 | 1439 | 38493 | 18470 | 4733 | 3133 | 1374 | 124 | 15783 |
| homo [symbol] - [symbol] | | | | | 279 | | | 0 | 0 | 0 | 1113 | 0 | 15 | 0 | 143 | 1019 |
| [symbol] - [symbol] | | | | | 277 | | | 0 | 0 | 442 | [symbol] | 143 | 29 | 0 | 0 | 2444 |
| Hpaec | | | | | 278 | 273 | | 110 | 124 | 87 | 2787 | 172 | 0 | 0 | 73 | 1917 |
| Ht [symbol] | | | | | 294 | | | 0 | 0 | 0 | 1285 | 0 | 113 | 82 | 109 | [symbol] |
| Ht [symbol] | | | | | 266 | | | 0 | 0 | 0 | 0909 | 0 | 0 | 13 | 0 | 7422 |
| [symbol] -11 | | | | | 239 | 336 | | 0 | 1002 | 0 | 8324 | 587 | 10 | 11 | 31 | 1434 |
| [symbol] - [symbol] | | | | | 238 | [symbol] | | 527 | 98 | 213 | 4743 | 1703 | 313 | 11 | 130 | 8438 |
| HT372- [symbol] | | | | | 234 | | | 158 | 0 | 0 | 4079 | 2761 | 734 | 0 | 52 | 2234 |
| [symbol] -7 | | | | | 233 | 233 | | 192 | 112 | 0 | 2948 | 0 | 134 | 64 | 0 | 8712 |
| [symbol] -6 | | | | | 231 | 231 | | 0 | 0 | 184 | [symbol] | 0 | 50 | 82 | 0 | [symbol] |
| [symbol] -2 | | | | | 239 | 279 | | 0 | 0 | 347 | 3473 | 0 | 50 | 50 | 171 | [symbol] |
| [symbol] -1 | | | | | 227 | 227 | | 308 | 52 | 0 | 1773 | 205 | 10 | 0 | 32 | 9127 |
| [symbol] - [symbol] | | | | | 322 | | | 41 | 0 | 1051 | 3212 | 0 | 183 | 133 | 40 | 2036 |
| [symbol] - [symbol] | | | | | 216 | | | 200 | 0 | 0 | 7296 | 876 | 0 | 131 | 0 | 1579 |
| [symbol] - [symbol] | | | | | 214 | | | 0 | 204 | 0 | 8474 | 0 | 1257 | 315 | 108 | 2857 |
| [symbol] - [symbol] | | | | | 213 | | | 35 | 8 | 0 | [symbol] 1 | 0 | 123 | 0 | 0 | [symbol] |
| [symbol] - [symbol] | | | | | 212 | | | 0 | 93 | 243 | [symbol] | 196 | [symbol] | 113 | 75 | 3834 |
| HCAEC | | | | | 211 | 211 | | 0 | 0 | 0 | 3921 | 1 | 190 | 0 | 28 | 1167 |
| [symbol] - [symbol] | | | | | 210 | | | 186 | 143 | 0 | 4763 | 0 | 873 | 109 | 182 | 1473 |
| [symbol] | | | | | 209 | | | 115 | 0 | 54 | 2295 | 0 | 1 [symbol] | 0 | 70 | 2718 |
| [symbol] - [symbol] | | | | | 205 | | | 236 | 50 | 125 | 2870 | 0 | 1785 | 72 | 15 | 2 [symbol] |
| [symbol] - [symbol] | | | | | 203 | | | 124 | 15 | 329 | 3074 | 183 | 53 | 38 | 0 | 831 |
| [symbol] - [symbol] | | | | | 201 | | | 0 | 0 | 194 | 27 [symbol] | 0 | 83 | 101 | 7 | 2100 |
| [symbol] - [symbol] | | | | | 1 [symbol] | | | 1 [symbol] | 35 | 0 | 3811 | 0 | 154 | 63 | 0 | 1438 |
| [symbol] - [symbol] | | | | | 197 | | | 33 | 49 | 2102 | 2 [symbol] | 125 [symbol] | 183 | 113 | 0 | [symbol] |
| HEPM 34 TOPS1 [symbol] | | | | | [symbol] | | | 444 | 135 | 213 | 1914 | 0 | 364 | 0 | 70 | 3531 |
| [symbol] - [symbol] | | | | | 183 | | | 0 | 444 | 135 | 213 | 0 | 349 | 71 | 0 | 712 |
| [symbol] - [symbol] | | | | | 81 | | | 0 | 87 | 0 | 1834 | 0 | 0 | 172 | 0 | 1302 |
| [symbol] - [symbol] | | | | | 86 | | | 409 | 3 [symbol] | 447 | 1283 | 0 | 1374 | 867 | 31 | 13412 |
| [symbol] - [symbol] | | | | | 87 | | | 0 | 213 | 181 | 1330 | 0 | 977 | 476 | 112 | [symbol] |
| [symbol] - [symbol] | | | | | [symbol] | | | 155 | 184 | 3427 | 2343 | 0 | [symbol] | 281 | [symbol] | 4283 |
| [symbol] - [symbol] | | | | | [symbol] 3 | | | 149 | 113 | 105 | 34 | 0 | 0 | 0 | 21 | 834 |
| [symbol] - [symbol] | | | | | 81 | | | 0 | 83 | 204 | 1747 | 18 | 382 | [symbol] | 0 | 2902 |
| [symbol] - [symbol] | | | | | 49 | | | 0 | 78 | 290 | 5454 | 1233 | 275 | 218 | 0 | 1413 |
| HELA- [symbol] -031 [symbol] | | | | 79 | | | | 213 | 587 | 121 | 3227 | 805 | 81 | 190 | 0 | 8484 |
| HELA- [symbol] -031 [symbol] | | | | 81 | | | | 72 | 174 | 0 | 3329 | 0 | 0 | [symbol] | 0 | 5479 |
| HELA- [symbol] -031 [symbol] | | | | 83 | | | | 0 | 253 | 215 | 2 [symbol] | 204 | 0 | 315 | 0 | [symbol] |
| HELA- [symbol] -031 [symbol] | | | | 84 | | | | 83 | 0 | 257 | 2475 | 0 | 0 | 296 | 0 | [symbol] 47 |
| HELA- [symbol] -031 [symbol] | | | | [symbol] | | | | 140 | 340 | 0 | 9084 | 37 [symbol] | 0 | 247 | 0 | 4351 |
| HELA- [symbol] -031 [symbol] | | | | [symbol] | | | | 342 | 203 | 0 | 6775 | 0 | 94 | 0 | 183 | 7038 |
| HELA- [symbol] -031 [symbol] | | | | 82 | | | | 33 | 813 | 616 | 8034 | 0 | 184 | 0 | 0 | 7091 |
| HELA- [symbol] -031 [symbol] | | | | [symbol] | | | | 134 | 143 | 234 | 4109 | 0 | 0 | 0 | 0 | 3438 |
| HELA- [symbol] -031 [symbol] | | | | [symbol] | | | | 43 | 0 | 384 | 1916 | 0 | 103 | 47 | 0 | 3000 |
| H [symbol] | | | | 146 | | | | 0 | 18 | 8702 | 747 | 1508 | 1734 | 34 | 0 | 1485 |
| H [symbol] | | | | 148 | | | | 0 | 74 | 9 [symbol] | 1557 | 241 | 11 | 0 | 34 | 2372 |
| SA [symbol] 19 | | | | 150 | | | | 0 | 74 | 9783 | 1557 | 241 | 71 | 0 | 34 | 2372 |
| [symbol] | | | | 152 | | | | 0 | 138 | [symbol] | [symbol] 30 | 4181 | 818 | 0 | 0 | 3485 |
| [symbol] | | | | 154 | | | | 91 | 109 | 34338 | [symbol] | 300 | 1174 | [symbol] | 194 | 8211 |
| SF [symbol] | | | | 154 | | | | 242 | 0 | 3482 | 0 | [symbol] | 37 | 102 | 0 | 1189 |
| SF- [symbol] | | | | 154 | | | | 52 | 0 | 1743 | 201 | 0 | 87 | 3 | 27 | 937 |
| CCRF-CEM | | | | 150 | | | | 0 | 0 | [symbol] 83 | 443 | 3 | 247 | 0 | 121 | 1419 |
| DY- [symbol] | | | | 152 | | | | [symbol] | 0 | 1041 | 173 | 6033 | 184 | 302 | 87 | [symbol] |
| HCT 116 | | | | 184 | | | | 0 | 20 | 1437 | 817 | 372 | 0 | 0 | 1437 | 791 |
| [symbol] 1 | | | | [symbol] | | | | 279 | 236 | [symbol] 781 | 1322 | 0 | 782 | 0 | 0 | [symbol] |
| T [symbol] | | | | [symbol] | | | | [symbol] | [symbol] | [symbol] | 4542 | 3083 | 4163 | 138 | 0 | 3600 |
| T470 | | | | [symbol] | | | | 0 | 236 | 13838 | 3870 | [symbol] | 2368 | 454 | 0 | 1 [symbol] |
| [symbol] | | | | 171 | | | | 43 | 0 | 0 | 1622 | [symbol] | 0 | 61 | 0 | 2573 |
| [symbol] 1441 [symbol] | | | | 181 | | | | 0 | 264 | 0 | 0 | 1781 | 0 | 1 | 128 | 543 |
| [symbol] | | | | 183 | | | | 28 | 0 | 0 | 321 | 111 | 0 | 0 | 0 | 384 |
| [symbol] | | | | 184 | | | | 114 | 0 | 141 | 2325 | 0 | 0 | 136 | 0 | 3700 |
| [symbol] | | | | 194 | | | | 218 | 0 | 1383 | 3226 | 0 | 0 | 101 | 130 | 3434 |
| [symbol] | | | | 198 | | | | 0 | 172 | 10072 | 1171 | [symbol] | 147 | 130 | 83 | 1431 |
| [symbol] | | | | 200 | | | | [symbol] | 0 | 3877 | 0 | 2173 | 364 | 0 | 183 | 4827 |
| [symbol] | | | | 202 | | | | 100 | 0 | [symbol] | 402 | 215 | 140 | 0 | 0 | 3477 |
| [symbol] | | | | 204 | | | | 112 | 0 | 0 | 300 | 0 | 79 | 252 | 134 | 438 |
| [symbol] | | | | 20 [symbol] | | | | 0 | 300 | 813 | 237 | 216 | 0 | 0 | 0 | 1376 |
| [symbol] | | | | 20 [symbol] | | | | 1140 | 0 | 2134 | 2643 | 526 | 800 | 0 | 197 | 8071 |
| [symbol] | | | | 216 | | | | 0 | 0 | 382 | 1884 | 0 | 25 | 143 | 8 | 3126 |
| [symbol] | | | | 219 | | | | 0 | 0 | 901 | 3083 | 414 | 626 | 184 | 0 | 3255 |
| C [symbol] 1441- [symbol] | | | | 220 | | | | 0 | 154 | 0 | 177 | 0 | 0 | 8 | 71 | 1173 |
| [symbol] -1 | | | | 221 | | | | 18 | [symbol] | 0 | 2117 | 0 | 0 | [symbol] 1 | 84 | 2344 |
| [symbol] -2 | | | | 223 | | | | [symbol] | 0 | 45 | 1182 | 104 | 0 | 133 | 0 | 2280 |
| [symbol] -4 | | | | 225 | | | | 0 | 230 | 0 | 622 | 0 | 0 | 34 | 0 | 1273 |
| [symbol] | | | | 241 | | | | 0 | 0 | 1700 | 1800 | 183 | 136 | 216 | 0 | 2325 |
| HCL T-4 | | | | 242 | | | | [symbol] | 143 | [symbol] 63 | 1036 | 3006 | 183 | 160 | 10 | 1276 |
| [symbol] | | | | 243 | | | | 0 | 0 | 32076 | 1884 | [symbol] | 311 | 300 | 0 | [symbol] |
| [symbol] | | | | 244 | | | | 0 | 228 | 14080 | 2061 | 0 | 64 | 215 | 0 | 4264 |
| [symbol] | | | | 245 | | | | 0 | 1087 | 77132 | 1403 | 0 | 700 | 225 | 1 | 3648 |
| [symbol] 1226 | | | | 246 | | | | 0 | 0 | 0 | 011 | 838 | 57 | 0 | 7 | 34 |
| [symbol] TCC | | | | 247 | | | | 0 | 0 | 4447 | 255 | 0 | 168 | 98 | 0 | 2344 |
| SR | | | | 248 | | | | 0 | 183 | 18413 | 954 | 1882 | 638 | 17 | 0 | 2103 |
| DVC- [symbol] | | | | 249 | | | | 0 | 144 | 8806 | 1324 | 168 | 0 | 80 | 218 | 4234 |
| HCT-15 | | | | 250 | | | | 129 | 0 | 45225 | 1488 | 201 | 500 | 0 | 95 | 8063 |
| [symbol] -4 | | | | 251 | | | | 171 | 0 | 2381 | 54 | 0 | 180 | 0 | 187 | 98323 |
| UC-31 | | | | 252 | | | | 208 | 0 | 307 | 460 | 0 | 38 | 0 | 131 | 1431 |
| DVC-AR-6 | | | | 253 | | | | 0 | 10 | 577 | 3408 | 0 | 1477 | 0 | 0 | 4318 |
| [symbol] | | | | 254 | | | | 486 | 0 | 6634 | 173 | 0 | 0 | 322 | 1821 | 4185 |
| DVC-AR- [symbol] | | | | 255 | | | | 181 | 94 | 17088 | 486 | 0 | 54 | 128 | 0 | 1781 |
| [symbol] | | | | 256 | | | | 0 | 2036 | 82875 | 1863 | 1303 | 1383 | 0 | 134 | 5348 |
| [symbol] | | | | 257 | | | | 140 | 0 | 32874 | 1309 | 176 | 178 | 0 | 326 | 4434 |
| [symbol] -2 | | | | 258 | | | | 0 | 187 | 4482 | 224 | 0 | 0 | 48 | 547 | 1206 |
| SLOV-3 | | | | 259 | | | | 0 | 13 | 4761 | 313 | 681 | 681 | 0 | 71 | 1009 |
| SX-VCL-4 | | | | 260 | | | | 0 | 101 | 3676 | 172 | 38 | 29 | 0 | 41 | 1811 |
| [symbol] | | | | 261 | | | | [symbol] | 0 | 73832 | 1603 | 821 | 927 | 456 | 174 | 4154 |
| [symbol] | | | | 262 | | | | 187 | 0 | 14342 | 144 | 0 | 0 | 34 | 0 | 1278 |
| X-342 | | | | 263 | | | | 0 | 118 | 36631 | 926 | 1876 | 432 | 74 | 63 | 2880 |
| LACC-257 | | | | 264 | | | | 0 | 83 | 8654 | 1800 | 175 | 81 | 108 | 6 | 1257 |
| M14 | | | | 265 | | | | 0 | 21 | 1254 | 873 | 3737 | 0 | 121 | 0 | 2308 |
| MCF7 | | | | 267 | | | | 128 | 258 | 23780 | 6300 | 1000 | 0 | 215 | 0 | 14382 |
| MDA- [symbol] -425 | | | | 268 | | | | 0 | 0 | 8781 | 383 | 0 | 147 | 250 | 136 | 4085 |
| MT276 | | | | 270 | | | | 30 | 36 | 0 | 1064 | 0 | 67 | 170 | 25 | 3872 |
| MDA-N | | | | 271 | | | | 0 | 0 | 14736 | 415 | 0 | 441 | 336 | 0 | 4136 |
| [symbol] | | | | 273 | | | | 11 | 341 | 8084 | 4474 | 0 | 1320 | 0 | 345 | 12179 |
| [symbol] | | | | 280 | | | | 25 | 360 | 15640 | 6370 | 1854 | 2113 | 1061 | 0 | 11610 |
| WT838 [symbol] TPA [symbol] 823 | | | | 300 | | | | 71 | 0 | 0 | 8304 | 0 | 0 | 40 | 483 | 786 |
| HELA-EXP- [symbol] | | | | 313 | | | | 37 | 42 | 0 | 4774 | 0 | 0 | 185 | 97 | 3084 |
| [symbol] | | | | 322 | | | | 344 | 0 | 616 | 6852 | 16719 | 0 | 800 | 0 | 4427 |
| HT347 | | | | 323 | | | | [symbol] | 0 | 50 | 4108 | 170 | 377 | 142 | 83 | 8447 |
| [symbol] | | | | 324 | | | | 942 | 94 | 0 | 8137 | 8287 | 481 | 385 | 170 | 1663 |
| MCL-H [symbol] | | | | 336 | | | | [symbol] | 31 | 11483 | 1317 | 1815 | 8 | 171 | 0 | 0 |
| HOP-42 | | | | 337 | | | | 0 | 0 | 10827 | 3205 | 0 | 226 | 138 | 0 | 3071 |
| MOA-MB-231 | | | | 338 | | | | [symbol] | 1127 | 138008 | 18086 | 38722 | 8586 | 1087 | 45 | 13418 |
| U251 | | | | 339 | | | | [symbol] | 1786 | 720047 | 9036 | 80293 | 30873 | 1374 | 0 | 19432 |
| PT [symbol] | | | | 340 | | | | 0 | 112 | 8025 | 778 | 0 | 446 | 136 | 37 | 2086 |
| PC-3 | | | | 341 | | | | 673 | 215 | 29876 | 7064 | 78117 | 1417 | 1087 | 0 | 9562 |
| HCC-2000 | | | | 343 | | | | 0 | 3 | 7508 | 1213 | 708 | 179 | 829 | 0 | 3085 |
| [symbol] | | | | 345 | | | | 80 | 6 | 32834 | 1180 | 5491 | 27 | 343 | 0 | 5447 |
| HT182 | | | | 346 | | | | 48 | 0 | 1998 | 1007 | 0 | 13 | 381 | 0 | 3808 |
| COLO 20 [symbol] | | | | 347 | | | | 17 | 96 | 3856 | 946 | 834 | 0 | 481 | 0 | 940 |
| HT316 | | | | 348 | | | | 1 | 0 | 512 | 6443 | 0 | 72 | 0 | 0 | 2341 |
| MO4-12 | | | | 349 | | | | 0 | 81 | 9886 | 3008 | 913 | 0 | 188 | 0 | 1130 |
| HT151 | | | | 350 | | | | 32 | 302 | 252 | 7132 | 0 | 782 | 306 | 22 | 4497 |
| A438 | | | | 351 | | | | 137 | 11 | 8123 | 3278 | 2335 | 0 | 308 | 0 | 2252 |
| HT383 | | | | 352 | | | | 235 | 131 | 0 | 14919 | 186 | 0 | 183 | 0 | 1887 |
| PGG [symbol] | | | | 353 | | | | 350 | 0 | 8021 | 2130 | 3003 | 112 | 219 | 0 | 1887 |
| T [symbol] | | | | 355 | | | | 2370 | 1831 | 58083 | 4438 | 38183 | 4039 | 184 | 173 | 6282 |
| [symbol] | | | | 357 | | | | 1422 | 1373 | 942135 | 14400 | 51277 | 9086 | 947 | 84 | 6236 |
| MS 8787 | | | | 358 | | | | 184 | 48 | 21180 | 1286 | 1187 | 1033 | 180 | 12 | 628 |
| HT313 | | | 50 | | | | | 0 | 0 | 707 | 2163 | 0 | 600 | 338 | 0 | 14318 |
| HT344 | | | 52 | | | | | 144 | 0 | 947 | 3338 | 0 | 98 | 160 | 0 | 18288 |
| HT130 | | | 53 | | | | | 174 | 0 | 0 | 3229 | 0 | 84 | 307 | 27 | 9584 |
| HT156 | | | 54 | | | | | 0 | 0 | 13 | 2383 | 0 | 0 | 80 | 0 | 18006 |
| HT163 | | | 58 | | | | | 376 | 0 | 118 | 1859 | 0 | 412 | 181 | 117 | 9434 |
| HT170 | | | 60 | | | | | 713 | 0 | 2417 | 9484 | 1368 | 0 | 710 | 0 | 13123 |
| HT172 | | | 62 | | | | | 111 | 0 | 95 | 2328 | 0 | 0 | 29 | 0 | [symbol] |
| HT178 | | | 63 | | | | | 384 | 34 | 83 | 3079 | 0 | 0 | 0 | 0 | 8428 |
| HT179 | | | 64 | | | | | 0 | 78 | 147 | 3414 | 0 | 821 | 40 | 0 | 6827 |
| HT184 | | | 65 | | | | | [symbol] | 0 | 374 | 2820 | 0 | 0 | 907 | 0 | 12319 |
| HT180 | | | 66 | | | | | 43 | 122 | 4371 | 3000 | 0 | 0 | 0 | 48 | 3831 |
| HT188 | | | 67 | | | | | 0 | 118 | 0 | 1861 | 0 | 0 | 340 | 182 | 2359 |
| HT190 | | | 68 | | | | | 43 | 638 | 2823 | 3580 | 0 | 984 | 83 | 78 | 8023 |
| HT143 | | | 69 | | | | | 327 | 0 | 0 | 2961 | 0 | 0 | 182 | 0 | 432 |
| HT1 [symbol] 0 | | | 70 | | | | | 382 | 71 | 1386 | 8686 | 0 | 209 | 388 | 0 | 1551 |
| HT144 | | | 71 | | | | | 84 | 88 | 2044 | 4734 | 0 | 0 | 230 | 0 | 428 |
| HT227 | | | 72 | | | | | 12 | 0 | 215 | 3473 | 0 | 1015 | 484 | 0 | 2054 |
| HT302 | | | 73 | | | | | 143 | 0 | 2818 | 7388 | 1860 | 4134 | 721 | 174 | 25415 |
| HT314 | | | 74 | | | | | 230 | 0 | 674 | 6547 | 0 | 838 | 340 | 0 | 15481 |
| HT317 | | | 76 | | | | | 285 | 0 | 348 | 7398 | 705 | 978 | 585 | 235 | 15104 |
| [symbol] | | | 77 | | | | | 0 | 0 | 0 | [symbol] | 2 | 0 | 52 | 0 | 87 |
| HT323 | | | 79 | | | | | 0 | 0 | [symbol] | [symbol] | 0 | [symbol] | [symbol] | 0 | 9443 |
| HT327 | | | 80 | | | | | 0 | 83 | 230 | [symbol] | 1133 | 0 | [symbol] | [symbol] | [symbol] |
| HT336 | | | 82 | | | | | 0 | 0 | 0 | [symbol] | 0 | [symbol] | 0 | 0 | [symbol] |
| HT1 [symbol] | | | 83 | | | | | 4 [symbol] | 5 | 93 | 0 | 0 | [symbol] | 0 | 132 | 1344 |
| HT3 [symbol] | | | 87 | | | | | [symbol] | 15 [symbol] | 1 [symbol] 0 | 1429 | 0 | 1017 | 0 | 0 | 3434 |
| HT311 | | | 170 | | | | | 23 [symbol] | 0 | 0 | 317 [symbol] | 0 | 130 | [symbol] | 117 | [symbol] |
| HT [symbol] | | | 185 | | | | | [symbol] 73 | 0 | 0 | 11 [symbol] | 1414 | 0 | [symbol] | 0 | [symbol] |
| HT1 [symbol] | | | 187 | | | | | 230 | 117 | 0 | 0 | 0 | 0 | 1 [symbol] | 1 [symbol] | 144 |
| HT [symbol] | | | 189 | | | | | 303 | 0 | 0 | 1304 | [symbol] | 0 | 3 [symbol] 1 | 0 | 4 [symbol] |
| HT372 | | | 191 | | | | | 43 | 220 | 0 | 2713 | 0 | 2 [symbol] | 1 [symbol] | 144 | 43 [symbol] |
| TCOP | | | 207 | | | | | [symbol] 4 | 17 | 0 | 14 [symbol] 0 | 0 | 0 | 10 | 0 | 3112 |
| HT1 [symbol] 0 | | | 215 | | | | | 0 | 0 | [symbol] 0 | 13 [symbol] | 304 | 0 | 40 | 0 | 1331 |
| HT [symbol] 07 | | | 217 | | | | | 0 | [symbol] 0 | 0 | 3222 | 0 | 44 | 14 [symbol] | 0 | 41 [symbol] |
| HT3 [symbol] 4 | | | 224 | | | | | 343 | 0 | 0 | [symbol] | 13719 | 127 | 327 | 11 [symbol] | 4916 |
| HT370 | | | 225 | | | | | 114 | 0 | 0 | 1343 | 0 | 0 | 0 | 0 | [symbol] |
| HT371 | | | 228 | | | | | 170 | [symbol] | 32 | [symbol] 71 | 0 | 0 | 12 [symbol] | 0 | [symbol] 22 |
| HT377 | | | 230 | | | | | 30 | 181 | 122 | [symbol] | [symbol] 7 | 135 | [symbol] 3 | 0 | [symbol] |
| HT [symbol] | | | 234 | | | | | 1 [symbol] | [symbol] 1 | 0 | 4305 | 142 | 820 | [symbol] | 132 | [symbol] |
| [symbol] | | | 2 [symbol] 1 | | | | | 0 | 0 | 103 | [symbol] | 0 | 30 | 41 | 0 | 283 |
| HT334 | | | [symbol] | | | | | 0 | 127 | 0 | 8791 | 1302 | [symbol] | 123 | 0 | [symbol] |
| HT334 | | | 301 | | | | | [symbol] | 52 | 0 | 2712 | 0 | 185 | 341 | 279 | [symbol] |
| HT [symbol] | | | 315 | | | | | 1 [symbol] 4 | 0 | 22 [symbol] | 322 | [symbol] | 107 | 31 | 308 | 3177 |
| HT [symbol] | | | 317 | | | | | 83 | 2474 | 0 | [symbol] | 0 | [symbol] 01 | [symbol] | 0 | 77 [symbol] |
| HT312 | | | 318 | | | | | 309 | [symbol] | 1 [symbol] 4 | [symbol] | 0 | 17 | [symbol] | 180 | [symbol] 743 |
| HT182 | | | 325 | | | | | 415 | 0 | 22 [symbol] | 2191 | 0 | 0 | 0 | 0 | 20 |
| HT [symbol] | | | 354 | | | | | [symbol] | 100 | 0 | 3119 | 0 | 0 | 0 | 0 | 2471 |
| HT1 [symbol] 7 | | | 3 [symbol] 0 | | | | | 4 [symbol] | 0 | 0 | 4418 | [symbol] | [symbol] | [symbol] | 0 | 145 |
| T-470 | 143 | | | | | | | 437 | 128 | 35100 | [symbol] | 174 [symbol] 70 | 3414 | 112 | 0 | [symbol] |
| [symbol] | [symbol] | | | | | | | 485 | 135 | [symbol] 0419 | [symbol] 015 | 157 [symbol] | 1441 | 123 | 114 | 4 [symbol] 1 |
| MO [symbol] - [symbol] -435 | 154 | | | | | | | 79 | 0 | 142 [symbol] 4 | 404 [symbol] | [symbol] | 2831 | 197 | 0 | [symbol] |
| MO [symbol] - [symbol] -231 | 157 | | | | | | | [symbol] 15 | 282 | [symbol] | [symbol] | [symbol] | 7911 | 328 | 441 | 4471 |
| [symbol] | 153 | | | | | | | 235 | 125 | 122345 | 4402 | 291883 | 3072 | 334 | 43 | 9383 |
| MCF- [symbol] | 152 | | | | | | | 464 | 0 | 82988 | 3783 | 172347 | 408 | 234 | 71 | 4902 |
| MCF7 | 151 | | | | | | | 0 | 90 | 23491 | 3643 | 218251 | 402 | 407 | 54 | 6604 |
| M14 | 149 | | | | | | | 34 | 0 | [symbol] | 5725 | 99836 | 734 | 734 | 78 | [symbol] |
| UACC-2 [symbol] | 148 | | | | | | | 0 | 130 | [symbol] | 4088 | 49506 | 1177 | 1177 | 77 | 3942 |
| UACC- [symbol] | 147 | | | | | | | 9 | 33 | [symbol] | 8515 | 47918 | 123 | 1123 | 0 | [symbol] |
| [symbol] | 146 | | | | | | | 0 | 133 | [symbol] | 6315 | 14330 | 128 | 127 | 24 | [symbol] |
| VO-31 | 145 | | | | | | | 304 | 100 | 36141 | 6315 | 73374 | 120 | 120 | 27 | 4746 |
| [symbol] | 144 | | | | | | | 105 | 0 | 20849 | 9249 | 73371 | 1853 | 1055 | 11 | 2380 |
| [symbol] | 143 | | | | | | | 416 | 0 | 28587 | 2364 | 80993 | 198 | 190 | 182 | 2792 |
| [symbol] | 142 | | | | | | | 171 | 0 | 3464 | 2306 | 130029 | 27 | 27 | 132 | 7404 |
| HCT-13 | 141 | | | | | | | 0 | 47 | 22819 | 3813 | 194839 | 100 | 100 | 163 | 3750 |
| [symbol] | 140 | | | | | | | 140 | 130 | 12009 | 5785 | 137883 | 877 | 877 | 0 | 8273 |
| COLO [symbol] | 139 | | | | | | | 0 | 213 | 23499 | 5643 | 114681 | 942 | 942 | 74 | 4536 |
| Lax [symbol] | 138 | | | | | | | 158 | 1464 | 21811 | 3350 | 112345 | 1470 | 1476 | 0 | 7443 |
| [symbol] | 137 | | | | | | | 188 | 0 | 90087 | 3417 | 164917 | 796 | 796 | 210 | 9336 |
| TK- [symbol] | 136 | | | | | | | 0 | 137 | 11994 | 4148 | 19124 | 1900 | 1183 | 0 | [symbol] |
| HCT 11 [symbol] | 135 | | | | | | | 1080 | 90 | [symbol] | 2016 | 118724 | 162 | 182 | 283 | [symbol] |
| [symbol] | 134 | | | | | | | 225 | 0 | 7185 | 3038 | 103477 | 1314 | 3314 | 42 | 2382 |
| HCC- [symbol] | 132 | | | | | | | 0 | 180 | 28315 | 4509 | 127191 | 485 | 495 | 0 | 1400 |
| ACM | 131 | | | | | | | 0 | 130 | 17047 | 4256 | 97808 | 473 | 473 | 144 | 1600 |
| PC-3 | 130 | | | | | | | 100 | 104 | 28733 | 2713 | 90476 | 1017 | 1017 | 0 | 3677 |
| [symbol] | 129 | | | | | | | 134 | 204 | 8373 | 2502 | 98863 | 377 | 377 | 85 | 1233 |
| DU-145 | 128 | | | | | | | 0 | 70 | 34000 | 2431 | 17433 | 256 | 1258 | 0 | 10013 |
| C [symbol] | 127 | | | | | | | 0 | 141 | 8334 | 2645 | 64342 | 3136 | 3136 | 0 | 4275 |
| SR | 126 | | | | | | | 0 | 79 | 60187 | 2501 | 34429 | 1649 | 1619 | 47 | [symbol] |
| A [symbol] | 125 | | | | | | | 0 | 153 | 8483 | 2800 | 31346 | 19 | 15 | 9 | 10134 |
| RPM [symbol] | 124 | | | | | | | 34 | 0 | 28456 | 2614 | 12834 | 102 | 102 | 163 | 24438 |
| SN12C | 123 | | | | | | | 0 | 0 | 80 | 7382 | 38811 | 142 | 142306 | 43 | 3934 |
| [symbol] | 122 | | | | | | | 813 | 100 | 34753 | 8301 | 489 | 365 | 1346 | 173 | 9433 |
| MOLT-4 | 121 | | | | | | | 0 | 0 | 825 | 5372 | 83742 | 1349 | 4613 | 0 | 8579 |
| OVCAR-5 | 120 | | | | | | | 425 | 0 | 908 | 4872 | 41219 | 4613 | 2277 | 171 | 3791 |
| X- [symbol] | 119 | | | | | | | 254 | 0 | 820 | 4997 | 198477 | 2277 | 8057 | 70 | 6240 |
| OVCAR-4 | 118 | | | | | | | 854 | 246 | 10918 | 2514 | 133148 | 4057 | 9077 | 9 | 3661 |
| CCAF-CON | 117 | | | | | | | 0 | 0 | 21540 | 3482 | 113796 | 9077 | 1905 | 9 | 6605 |
| OVCAR-3 | 116 | | | | | | | 464 | 37 | 14296 | 2885 | 98833 | 1921 | 2383 | 0 | 5443 |
| [symbol] | 115 | | | | | | | 217 | 0 | 13067 | 1330 | 95279 | 2383 | 2098 | 0 | 13804 |
| MOP-42 | 114 | | | | | | | 404 | 153 | 14168 | 3873 | 72198 | 2088 | 3313 | 0 | 8445 |
| SF-285 | 113 | | | | | | | 201 | 0 | 34834 | 4034 | 89199 | 3313 | 3808 | 6 | 4124 |
| [symbol] TCC | 112 | | | | | | | 1006 | 321 | 21130 | 2917 | 128199 | 3808 | 990 | 0 | 7211 |
| SF-295 | 111 | | | | | | | 0 | 0 | 44884 | 7405 | 134794 | 990 | 3557 | 416 | 4202 |
| NCA- [symbol] | 110 | | | | | | | 264 | 213 | 23877 | 1108 | 81138 | 3557 | 1164 | 113 | 6291 |
| SNS-75 | 109 | | | | | | | 232 | 0 | 33099 | 2890 | 1448017 | 1164 | 3814 | 253 | 1418 |
| MC [symbol] | 108 | | | | | | | 178 | 216 | 24583 | 1042 | 40575 | 3414 | 1006 | 5 | 4176 |
| SNB-19 | 107 | | | | | | | 0 | 335 | 27031 | 4126 | 69012 | 1006 | 1194 | 0 | 10000 |
| MCA-224 | 106 | | | | | | | 212 | 0 | 8081 | 2982 | 72818 | 1194 | 1803 | 0 | 5661 |
| SK-OV-3 | 105 | | | | | | | 138 | 14 | 23147 | 5630 | 17279 | 1103 | 4093 | 87 | 9011 |
| NCH-33 | 104 | | | | | | | 211 | 119 | 8406 | 2381 | 11321 | 4083 | 1944 | 298 | 12724 |
| [symbol] | 103 | | | | | | | 0 | 0 | 2151 | 2994 | 18030 | 9544 | 1016 | 171 | 8783 |
| BCVX | 102 | | | | | | | 319 | 432 | 8434 | 2107 | 2371 | 1016 | 1772 | 54 | 9100 |
| OVCAR-4 | 101 | | | | | | | 0 | 242 | 61716 | 4544 | 18131 | 1772 | 613 | 2151 | 12000 |
| MOP-42 | 100 | | | | | | | 62 | 122 | 6156 | 8395 | 5728 | 913 | 7072 | 0 | 4886 |
| [symbol] | 48 | | | | | | | 109 | 23 | 26183 | 7181 | 2989 | 3072 | 19079 | 0 | 4283 |
| [symbol] | 47 | | | | | | | 0 | 234 | 3123 | 8754 | 380 | 19079 | 4488 | 9 | 4773 |
| [symbol] | 46 | | | | | | | 315 | 280 | 19007 | 7246 | 120641 | 4968 | 634 | 9 | 6700 |
| TCOP | 76 | | | | | | | 0 | 0 | 22446 | 1182 | 122383 | 634 | 119 | 294 | 14122 |
| ASeq-1 | | | | | | | wt | 0 | 0 | 1880 | 6235 | 188916 | 116 | 341 | 8 | 110 |
| ASeq-3 | | | | | | | wt | 0 | 16 | 13008 | 83185 | 81496 | 341 | 3042 | 9 | 966 |
| ASeq-4 | | | | | | | wt | 0 | 48 | 11008 | 48136 | 0 | 3072 | 0 | 40 | 734 |
| ASeq-5 | | | | | | | wt | 0 | 22 | 2553 | 48247 | 0 | 0 | 1843 | 276 | 1182 |
| ASeq-7 | | | | | | | wt | 0 | 82 | 0 | 72997 | 0 | 1843 | 2142 | 243 | 9377 |
| EXVX-1 | | | | | | | [symbol] | 0 | 308 | 118 | 38990 | 0 | 2142 | 889 | 9 | 300 |
| EXVX-4 | | | | | | | [symbol] | 0 | 66 | 2509 | 99934 | 0 | 800 | 4794 | 9 | 1000 |
| EXVX-3 | | | | | | | [symbol] | 0 | 291 | 831 | 190881 | 0 | 4794 | 18041 | 122 | 824 |
| EXVX-6 | | | | | | | [symbol] | 0 | 225 | 2409 | 17798 | 0 | 46061 | 6764 | 129 | 3203 |
| EXVX-7 | | | | | | | [symbol] | 0 | 360 | 714 | 37831 | 0 | 6781 | 898 | 0 | 4100 |
| MCF-7-1 | | | | | | | wt | 0 | 16 | 49 | 74388 | 0 | 808 | 703 | 157 | 1196 |
| MCF-7-3 | | | | | | | wt | 0 | 1153 | 1208 | 41681 | 0 | 703 | 232 | 0 | 574 |
| MCF-7-4 | | | | | | | wt | 0 | 530 | 80 | 37949 | 0 | 332 | 0 | 0 | 891 |
| MCF-7-6 | | | | | | | wt | 0 | 234 | 0 | 32082 | 0 | 0 | 649 | 124 | 990 |
| MCF-7-7 | | | | | | | wt | 0 | 182 | 0 | 25875 | 0 | 948 | 0 | 91 | 997 |
| ADR_RES-1 | | | | | | | [symbol] | 0 | 0 | 1621 | 27895 | 0 | 199 | 2747 | 429 | 1776 |
| ADR_RES-3 | | | | | | | [symbol] | 0 | 264 | 0 | 88425 | 0 | 0 | 880 | 0 | 1127 |
| ADR_RES-4 | | | | | | | [symbol] | 0 | 144 | 20 | 46817 | 0 | 2747 | 1142 | 912 | 1423 |
| ADR_RES-5 | | | | | | | [symbol] | 0 | 811 | 1810 | 30547 | 0 | 990 | 1779 | 175 | 742 |
| ADR_RES-7 | | | | | | | [symbol] | 0 | 449 | 2457 | 27945 | 0 | 1142 | 1095 | 9 | 924 |
| WI 39-1 | | | | | | | wt | 0 | 0 | 218 | 87821 | 0 | 1779 | 11090 | 24 | 3042 |
| WI 39-3 | | | | | | | wt | 0 | 473 | 0 | 49671 | 0 | 1095 | 10250 | 0 | 1300 |
| WI 39-4 | | | | | | | wt | 0 | 453 | 4304 | 44081 | 0 | 19098 | 8330 | 110 | 1232 |
| WI 39-5 | | | | | | | wt | 0 | 382 | 3384 | 48543 | 0 | 10250 | 1404 | 0 | 1057 |
| WI 39-7 | | | | | | | wt | 0 | 0 | 1018 | 22973 | 0 | 8330 | 10729 | 0 | 1000 |
| H [symbol] -1 | | | | | | | [symbol] | 0 | 14 | 1422 | 38078 | 0 | 1404 | 857 | 0 | 1118 |
| H [symbol] -3 | | | | | | | [symbol] | 0 | 27 | 1554 | 40780 | 0 | 10729 | 764 | 189 | 847 |
| H [symbol] -4 | | | | | | | [symbol] | 0 | 247 | 385 | 41446 | 0 | 857 | 803 | 140 | 173 |
| H [symbol] -5 | | | | | | | [symbol] | 0 | 143 | 0 | 73772 | 0 | 764 | 1303 | 0 | 673 |
| H [symbol] -7 | | | | | | | [symbol] | 0 | 80 | 1254 | 40004 | 0 | 803 | 800 | 0 | 1152 |
| H1 2 [symbol] -1 | | | | | | | [symbol] | 0 | 148 | 3030 | 72779 | 0 | 1303 | 1805 | 0 | 949 |
| H1 2 [symbol] -3 | | | | | | | [symbol] | 0 | 0 | 864 | 26134 | 0 | 989 | 4889 | 181 | 441 |
| H1 2 [symbol] -4 | | | | | | | [symbol] | 0 | 687 | 149 | 36830 | 0 | 1906 | 14941 | 0 | 1922 |
| H1 2 [symbol] -5 | | | | | | | [symbol] | 0 | 1745 | 230 | 13450 | 0 | 4889 | 6836 | 0 | 1628 |
| H1 2 [symbol] -7 | | | | | | | [symbol] | 0 | 3504 | 1070 | 23000 | 0 | 14941 | 1127 | 64 | 577 |
| A [symbol] -2 | | | | | | | wt | 0 | 0 | 1823 | 34870 | 0 | 68341 | 1186 | 0 | 911 |
| BXVX-2 | | | | | | | [symbol] | 0 | 613 | 80 | 13387 | 0 | 1127 | 4336 | 115 | 1408 |
| MCT-114-1 | | | | | | | wt | 0 | 0 | 4427 | 82248 | 0 | 1186 | 1864 | 0 | 1781 |
| MCT-116-2 | | | | | | | wt | 0 | 1047 | 2782 | 122573 | 0 | 4238 | 7668 | 80 | 1283 |
| MT [symbol] -2 | | | | | | | [symbol] | 0 | [symbol] | 86 | 34958 | 0 | 1864 | 121 | 0 | 877 |
| SF [symbol] -1 | | | | | | | [symbol] | 0 | 17 | 64 | 22947 | 0 | 7886 | 2052 | 0 | 488 |
| SF [symbol] -2 | | | | | | | wt | 0 | 148 | 586 | 28348 | 0 | 121 | 4828 | 179 | 731 |
| SF [symbol] -1 | | | | | | | wt | 0 | 0 | 830 | 43018 | 0 | 2657 | 2529 | 0 | 1704 |
| SF [symbol] -2 | | | | | | | [symbol] | 0 | 182 | 21 | 48004 | 0 | 4828 | 8089 | 0 | 303 |
| OVCAR-1-1 | | | | | | | [symbol] | 0 | 0 | 3886 | 42904 | 0 | 3529 | 71791149 | 436 | 1914 |
| OVCAR-1-2 | | | | | | | wt | 0 | 239 | 1236 | 40099 | 0 | 8089 | 913 | 179 | 3434 |
| OVCAR-5-1 | | | | | | | wt | 0 | 1449 | 230 | 38089 | 0 | 7179 | 823 | 230 | 473 |
| OVCAR-5-2 | | | | | | | [symbol] | 0 | 1263 | 790 | 25744 | 0 | 1149 | 93 | 348 | 1084 |
| MCF-7-2 | | | | | | | [symbol] | 0 | 233 | 0 | 29122 | 0 | 913 | 0 | 0 | 973 |
| ADR RES-2 | | | | | | | wt | 0 | 1238 | 0 | 19243 | 0 | 923 | 144 | 63 | 1212 |
| H [symbol] -2 | | | | | | | [symbol] | 0 | 0 | 0 | 18042 | 0 | 83 | 5194 | 0 | 418 |
| SW440-1 | | | | | | | mpv E1 | 0 | 582 | 62 | 11909 | 0 | 0 | 5981 | 297 | 1302 |
| SW440-2 | | | | | | | [symbol] | 0 | 0 | 809 | 34850 | 0 | 344 | 7734 | 197 | 180 |
| H1 290-2 | | | | | | | [symbol] | 0 | 2207 | 1291 | 28070 | 0 | 5194 | 205 | 0 | 891 |
| C13A-1 | | | | | | | [symbol] | 0 | 1156 | 433 | 34405 | 0 | 5481 | 434 | 19 | 956 |
| C13A-2 | | | | | | | [symbol] | 0 | 2428 | 0 | 28147 | 0 | 7134 | 4953 | 113 | 934 |
| UPOS-1 | | | | | | | [symbol] | 0 | 817 | 0 | 60346 | 0 | 295 | 1297 | 47 | 2314 |
| UPOS-2 | | | | | | | [symbol] | 0 | 671 | 0 | 68820 | 0 | 484 | 3856 | 34 | 773 |
| Happ-1 | | | | | | | wt | 0 | 1380 | 8519 | 60213 | 0 | 4085 | 19711 | 0 | 989 |
| Happ-2 | | | | | | | wt | 0 | 275 | 1545 | 88867 | 0 | 1294 | 137 | 0 | 23036 |
| WI 38-1 | | | | | | | wt | 0 | 105 | 81 | 65770 | 0 | 856 | 13181 | 71 | 2020 |
| [symbol] -1 | | | | | | | | 0 | 134 | 241 | 23871 | 0 | 19711 | 0 | 67 | 973 |
| [symbol] -2 | | | | | | | | 0 | 2183 | 0 | 49133 | 0 | 13191 | 75 | 334 | 164 |
| [symbol] -3 | | | | | | | | 0 | 0 | 0 | 149384 | 0 | 0 | 752 | 0 | 1022 |
| [symbol] -4 | | | | | | | | 0 | 0 | 0 | 80008 | 0 | 75 | 232 | 82 | 480 |
| [symbol] -5 | | | | | | | | 0 | 0 | 0 | 1300070 | 0 | 257 | 82 | 9 | 463 |
| [symbol] -6 | | | | | | | | 0 | 0 | 131 | 50757 | 0 | 0 | 0 | 72 | 1066 |
| [symbol] -7 | | | | | | | | 0 | 0 | 0 | 82830 | 0 | 14493 | 14493 | 3535 | 1266 |
| [symbol] -8 | | | | | | | | 0 | 250 | 0 | 77383 | 0 | 1094 | 1094 | 0 | 1990 |
| [symbol] | | | | | | | | 0 | [symbol] | [symbol] | [symbol] | 0 | 7 [symbol] | 0 | 234 | 1 [symbol] |
| [symbol] -11 | | | | | | | | 0 | [symbol] | [symbol] 3 | 7 [symbol] | 0 | [symbol] 13 | 0 | 4 [symbol] | [symbol] 3 |
| [symbol] -12 | | | | | | | | 0 | [symbol] | 1 [symbol] | [symbol] | 0 | 142 [symbol] 1 | 0 | [symbol] 3 | 7 [symbol] |
| [symbol] | | | | | | | | 0 | 11 | [symbol] | 7 [symbol] | 0 | [symbol] 4 | 0 | 0 | 12 [symbol] |
| [symbol] -1 | | | | | | | | 0 | [symbol] | [symbol] | 47 [symbol] | 0 | [symbol] | 0 | 0 | 177 [symbol] |
| [symbol] -2 | | | | | | | | 0 | 34 | 0 | [symbol] | 0 | [symbol] 777 | 0 | 0 | [symbol] |
| [symbol] -3 | | | | | | | | 0 | [symbol] | 7 [symbol] | [symbol] 21 | 0 | 23 [symbol] | 0 | [symbol] | [symbol] |
| [symbol] -4 | | | | | | | | 0 | [symbol] | [symbol] | [symbol] | 0 | 1 [symbol] 11 | 0 | [symbol] | [symbol] 3 |
| [symbol] -5 | | | | | | | | 0 | 213 | [symbol] | [symbol] | 0 | 3713 | 0 | 0 | [symbol] |
| [symbol] -6 | | | | | | | | 0 | 52 | 541 | [symbol] 183 | 0 | [symbol] | 0 | 364 | [symbol] 1 |
| [symbol] | | | | | | | | 0 | [symbol] | 0 | 487 [symbol] | 0 | 0 | 0 | [symbol] | [symbol] |
| [symbol] | | | | | | | | 0 | [symbol] | 0 | [symbol] | 0 | [symbol] | 0 | 19 | 4 [symbol] |
| MCT-114-7 | | | | | | | [symbol] | 0 | [symbol] | 4742 | [symbol] 1 [symbol] 1 | 0 | [symbol] | 0 | 0 | [symbol] |
| MCT-114-8 | | | | | | | [symbol] | 0 | [symbol] | [symbol] | [symbol] | 0 | [symbol] | 0 | 0 | [symbol] |
| [symbol] -1 | | | | | | | [symbol] | 0 | 173 | [symbol] | 222 [symbol] | 0 | 1 [symbol] | 0 | 72 | 1 [symbol] |
| [symbol] -7 | | | | | | | [symbol] | 0 | [symbol] | 77 [symbol] | [symbol] 45 | 0 | 2302 | 0 | 0 | 1781 |
| [symbol] -8 | | | | | | | [symbol] | 0 | 187 | 1201 | 47 [symbol] 37 | 0 | 27 [symbol] | 0 | 315 | [symbol] |
| [symbol] -7 | | | | | | | [symbol] | 0 | 0 | [symbol] 0 | [symbol] 74 | 0 | [symbol] 0 | 0 | [symbol] | 441 |
| [symbol] -8 | | | | | | | [symbol] | 0 | 45 | [symbol] 43 | [symbol] | 0 | [symbol] | 0 | 0 | 4 [symbol] |
| [symbol] | | | | | | | [symbol] | 0 | 254 | 114 | [symbol] | 0 | 1 [symbol] | 0 | 124 | [symbol] |
| [symbol] | | | | | | | [symbol] | 0 | [symbol] 7 | 178 | [symbol] | 0 | 411 [symbol] | 0 | 0 | 1 [symbol] 1 |
| [symbol] -7 | | | | | | | [symbol] | 0 | 0 | 2013 | [symbol] 11 | 0 | [symbol] 40 | 0 | 141 | [symbol] |
| [symbol] -8 | | | | | | | [symbol] | 0 | 1 [symbol] | 1425 | [symbol] | 0 | 413 [symbol] | 0 | 12 [symbol] | 1371 |
| [symbol] | | | | | | | [symbol] | 0 | [symbol] | 0 | [symbol] 1 | 0 | [symbol] 34 | 0 | 140 | [symbol] |
| [symbol] | | | | | | | [symbol] | 0 | 0 | [symbol] 00 | [symbol] | 0 | [symbol] 734 | 0 | 0 | [symbol] |
| [symbol] | | | | | | | [symbol] | 0 | 1101 | [symbol] | [symbol] | 0 | [symbol] | 0 | 0 | 27 [symbol] |
| [symbol] | | | | | | | [symbol] | 0 | [symbol] | 474 | 13421 | 0 | 1 | 0 | 0 | [symbol] 71 |
| [symbol] | | | | | | | [symbol] | 0 | 20 [symbol] | 0 | [symbol] 7 | 0 | 234 | 0 | [symbol] | 342 |
| [symbol] | | | | | | | [symbol] | 0 | 104 | 0 | 23 [symbol] 03 | 0 | [symbol] 1 | 0 | 273 | 1 [symbol] |
| [symbol] | | | | | | | [symbol] | 0 | 1303 | 1034 | 31 [symbol] 2 | 0 | [symbol] | 0 | 2 [symbol] | [symbol] |
| [symbol] | | | | | | | [symbol] | 0 | [symbol] | 4 [symbol] 3 | [symbol] 435 | 0 | 117 [symbol] | 0 | [symbol] | 143 [symbol] |
| [symbol] | | | | | | | [symbol] | 0 | 1123 | 0 | [symbol] | 0 | 10 [symbol] | 0 | 330 | 87 [symbol] |
| [symbol] | | | | | | | [symbol] | 0 | 21 [symbol] | 7 [symbol] | [symbol] 01 [symbol] | 0 | [symbol] 1 | 0 | [symbol] 3 | 7 [symbol] 4 |
| [symbol] | | | | | | | [symbol] | 0 | 1000 | [symbol] 04 | [symbol] 11 | 0 | 0 | 0 | 0 | [symbol] 0 |
| [symbol] | | | | | | | [symbol] | 0 | 7777 | 323 | [symbol] 34 | 0 | [symbol] | 0 | 213 | 1883 |
| [symbol] | | | | | | | [symbol] | 0 | 7 [symbol] 0 | 1 [symbol] 7 | [symbol] 71 | 0 | [symbol] | 0 | 113 | [symbol] 7 [symbol] |
| [symbol] | | | | | | | [symbol] | 0 | 1323 | [symbol] | 33241 | 0 | 112 | 0 | 34 | [symbol] 34 |
| [symbol] | | | | | | | [symbol] | 0 | 44 | [symbol] 22 | [symbol] | 0 | [symbol] | 0 | 0 | [symbol] |
| [symbol] | | | | | | | [symbol] | 0 | 0 | [symbol] 30 | [symbol] 10 | 0 | 10 [symbol] | 0 | 0 | 1105 |
| [symbol] | | | | | | | [symbol] | 0 | [symbol] 6 | 0 | [symbol] | 0 | 142 | 0 | 0 | [symbol] |
| [symbol] | | | | | | | | [symbol] | 310 | 10715 | 147 [symbol] | [symbol] 13 | [symbol] | [symbol] | 0 | 74 [symbol] |
| [symbol] | | | | | | | | 0 | 0 | 1 [symbol] 4 | 137 [symbol] | [symbol] | 83 | [symbol] 1 | 0 | 2241 |
| [symbol] | | | | | | [symbol] | | 110 | 70 | 107 | 37 [symbol] | [symbol] 1 [symbol] | [symbol] | [symbol] 4 | 0 | 1 [symbol] |
| [symbol] | | | | | | | | 0 | 33 | 0 | 4330 | 1 [symbol] 7 | 22 | [symbol] | [symbol] | 7852 |
| [symbol] | | | | | | | | 0 | 30 | 0 | 4042 | [symbol] | 0 | 44 | 0 | [symbol] |
| [symbol] | | | | | | | | [symbol] | 115 | 251 | [symbol] | 14 [symbol] | 1123 | 11 [symbol] | 133 | [symbol] |
| [symbol] | | | | | | | | [symbol] | 145 | [symbol] 74 | 531 [symbol] | 0 | [symbol] 7 | 334 | 0 | [symbol] 3 |
| [symbol] | | | | | | 173 | | 105 | 0 | 0 | 14 [symbol] | [symbol] | 0 | 0 | 0 | 4 [symbol] 7 |
| [symbol] | | | | | | 17 [symbol] | | 45 | 11 [symbol] | 220 | 1 [symbol] 4 | [symbol] 4 | 0 | 0 | 0 | 2710 |
| [symbol] | | | | | | 177 | | 0 | 0 | 0 | 17 [symbol] | 0 | [symbol] | 0 | 103 | 3483 |
| [symbol] | | | | | | | | [symbol] | 0 | 0 | 2152 | 2734 | [symbol] | [symbol] 1 | 80 | [symbol] |
| [symbol] | | | | | | 237 | | [symbol] | 50 | 0 | [symbol] 1 | [symbol] | 41 [symbol] | 1 [symbol] | 77 | [symbol] |
| [symbol] | | | | | | | | [symbol] | 73 | [symbol] 21 | [symbol] 12 | 740 | 1 [symbol] | 181 | [symbol] | [symbol] |
| [symbol] | | | | | | | | 0 | 0 | [symbol] 1 | 3370 | 0 | [symbol] | 0 | 111 | [symbol] |
| [symbol] | | | | | | | | 0 | 0 | 0 | 4530 | 0 | 0 | 0 | 0 | 474 |
| [symbol] | | | | | | | | [symbol] | [symbol] 7 | 43 [symbol] | [symbol] | [symbol] | [symbol] | 37 [symbol] | 11 | [symbol] |
| [symbol] | | | | | | | | [symbol] | 0 | [symbol] 11 | 12 [symbol] 1 | [symbol] 2 | 4 [symbol] | 1030 | 24 [symbol] | [symbol] |
| [symbol] | | | | | | | | [symbol] | 0 | 7025 | 1 [symbol] 3 | [symbol] 43 | [symbol] 71 | 0 | 147 | 4771 |
| [symbol] | | | | | | | [symbol] | 0 | 0 | [symbol] 17 | [symbol] | 0 | [symbol] | 0 | 0 | 0 |
| [symbol] | | | | | | | [symbol] | 0 | 211 | 234 | [symbol] | 0 | [symbol] | 0 | [symbol] | 307 |
| [symbol] | | | | | | | [symbol] | 0 | 7 | 37 | [symbol] | 0 | 2413 | 0 | [symbol] | [symbol] |
| [symbol] | | | | | | | [symbol] | 0 | 1 [symbol] | 3 [symbol] 3 | [symbol] 7 [symbol] 7 | 0 | 1 [symbol] | 0 | [symbol] | 130 [symbol] |
| [symbol] | | | | | | | [symbol] | 0 | [symbol] | 0 | [symbol] | 0 | 0 | 0 | 0 | [symbol] 7 |
| [symbol] | | | | | | | [symbol] | 0 | [symbol] | [symbol] 78 | [symbol] | 0 | 1 [symbol] | 0 | 1 [symbol] | [symbol] |
| [symbol] | | | | | | | [symbol] | 0 | 0 | 41 | [symbol] | 0 | 187 | 0 | [symbol] | 1103 |
| [symbol] | | | | | | | [symbol] | 0 | 132 | 0 | 7 [symbol] 72 | 0 | [symbol] | 0 | 0 | [symbol] |
| [symbol] | | | | | | | [symbol] | 0 | 31 | 1 [symbol] 3 | 42341 | 0 | [symbol] | 0 | 0 | [symbol] |
| [symbol] | | | | | | | [symbol] | 0 | 470 | 0 | [symbol] 21 | 0 | 1 [symbol] | 0 | 0 | 12 [symbol] |
| [symbol] | | | | | | | [symbol] | 0 | [symbol] | [symbol] | 30 [symbol] | 0 | [symbol] 17 | 0 | 0 | [symbol] |
| [symbol] | | | | | | | [symbol] | 0 | [symbol] 3 [symbol] | 3 [symbol] 7 | [symbol] 12 | 0 | [symbol] | 0 | 0 | 1004 |
| [symbol] | | | | | | | [symbol] | 0 | 100 | [symbol] 34 | 1 [symbol] | 0 | [symbol] 0 | 0 | [symbol] | [symbol] |
| [symbol] | | | | | | | [symbol] | 0 | [symbol] 0 [symbol] | 4 [symbol] | [symbol] | 0 | 312 | 0 | [symbol] 15 | 1083 |
| [symbol] | | | | | | | [symbol] | 0 | 112 | 4 [symbol] | 27211 | 0 | 1 [symbol] 1 | 0 | 0 | 1 [symbol] |
| [symbol] | | | | | | | [symbol] | 0 | $0 | [symbol] | [symbol] | 0 | [symbol] | 0 | 30 | 1082 |
| [symbol] | | | | | | | [symbol] | 0 | 34 | 17 [symbol] | [symbol] 0 | 0 | 1507 | 0 | [symbol] | 1 [symbol] 34 |
| [symbol] | | | | | | | [symbol] | 0 | 211 | 1013 | [symbol] | 0 | 0 | 0 | [symbol] | [symbol] 7 |
| [symbol] | | | | | | | [symbol] | 0 | 1 [symbol] 1 | [symbol] | [symbol] | 0 | 1041 | 0 | 0 | 434 |
| [symbol] | | | | | | | [symbol] | 0 | 402 | [symbol] 4 | [symbol] | 0 | 1 [symbol] | 0 | [symbol] | [symbol] 11 |
| [symbol] | | | | | | | [symbol] | 0 | 103 | 41 [symbol] | [symbol] | 0 | [symbol] 3 | 0 | 0 | 477 |
| [symbol] | | | | | | | [symbol] | 0 | 3 [symbol] | [symbol] 7 | [symbol] | 0 | [symbol] | 0 | 0 | [symbol] |
| [symbol] | | | | | | | [symbol] | 0 | 1 [symbol] 2 | 0 | [symbol] | 0 | 32 | 0 | [symbol] 4 | [symbol] 1 |
| [symbol] | | | | | | | [symbol] | 0 | 72 | [symbol] 3 | [symbol] | 0 | 11 [symbol] | 0 | 72 | [symbol] |
| [symbol] | | | | | | | [symbol] | 0 | 0 | 0 | [symbol] 441 | 0 | 40 [symbol] | 0 | 0 | [symbol] 01 |
| [symbol] | | | | | | | [symbol] | 0 | 17 [symbol] 7 | 444 | [symbol] 371 | 0 | [symbol] 777 | 0 | [symbol] | [symbol] |
| [symbol] | | | | | | | [symbol] | 0 | 7 [symbol] 7 | [symbol] 0 | [symbol] 0 | 0 | [symbol] | 0 | 0 | 1010 |
| [symbol] | | | | | | | [symbol] | 0 | 12 [symbol] | [symbol] 4 | [symbol] 7 | 0 | [symbol] | 0 | [symbol] | 1571 |
| [symbol] | | | | | | | [symbol] | 0 | [symbol] 4 | 1207 | 21434 | 0 | [symbol] | 0 | 79 | [symbol] 0 |
| [symbol] | | | | | | | [symbol] | 0 | 1 [symbol] 3 | 0 | 4417 [symbol] | 0 | [symbol] 73 | 0 | 32 | 185 |
| [symbol] | | | | | | | [symbol] | 0 | 112 [symbol] | 1 [symbol] | [symbol] | 0 | [symbol] | 0 | 18 | 13 [symbol] |
| [symbol] | | | | | | | [symbol] | 0 | 1 [symbol] | 711 | [symbol] | 0 | 0 | 0 | 83 | [symbol] |
| [symbol] | | | | | | | [symbol] | 0 | [symbol] 0 | 37 [symbol] | 21 [symbol] | 0 | [symbol] | 0 | 30 | 330 |
| [symbol] | | | | | | | [symbol] | 0 | 434 | 0 | 23074 | 0 | 0 | 0 | 123 | 412 |
| [symbol] | | | | | | | [symbol] | 0 | [symbol] | [symbol] 47 | [symbol] | 0 | [symbol] | 0 | 0 | 1217 |
| [symbol] | | | | | | | [symbol] | 0 | [symbol] | [symbol] 4 | [symbol] | 0 | [symbol] | 0 | 302 | [symbol] |
| [symbol] | | | | | | | [symbol] | 0 | 187 | 3251 | [symbol] | 0 | [symbol] | 0 | 120 | [symbol] |
| [symbol] | | | | | | | [symbol] | 0 | 44 | [symbol] | [symbol] 411 | 0 | [symbol] | 0 | 0 | 1700 |
| [symbol] | | | | | | | [symbol] | 0 | [symbol] | 0 | 1 [symbol] 1 | 0 | 112 | 0 | 0 | 57 [symbol] |
| [symbol] | | | | | | | [symbol] | 0 | 0 | [symbol] | 4 [symbol] | 0 | [symbol] 73 | 0 | 0 | [symbol] |
| [symbol] | | | | | | | [symbol] | 0 | 72 | 4 [symbol] | [symbol] | 0 | 101 [symbol] 1 | 0 | [symbol] 1 | [symbol] |
| [symbol] | | | | | | | [symbol] | 0 | 111 | 14 [symbol] | 47 [symbol] | 0 | [symbol] | 0 | 33 | 7 [symbol] |
| [symbol] | | | | | | | [symbol] | 0 | 1 | 22 [symbol] | [symbol] | 0 | 41 [symbol] | 0 | 0 | [symbol] 0 [symbol] |
| [symbol] | | | | | | | [symbol] | 0 | [symbol] 0 | [symbol] 1 | [symbol] 41 [symbol] | 0 | [symbol] | 0 | 0 | 1182 |
| [symbol] | | | | | | | [symbol] | 0 | 0 | [symbol] | [symbol] 7 [symbol] | 0 | [symbol] 7 [symbol] | 0 | 0 | [symbol] |
| [symbol] | | | | | | | | 0 | 0 | 11 [symbol] 3 | 134780 | 0 | 4570 | 0 | 0 | [symbol] 3 |
| [symbol] | | | | | | | | 0 | 0 | [symbol] | 221237 | 0 | 13 [symbol] | 0 | 213 | 7 [symbol] |
| [symbol] | | | | | | | | 0 | 0 | 0 | [symbol] | 0 | [symbol] 1 | 0 | 114 | 11 [symbol] |
| [symbol] | | | | | | | | 0 | 0 | 0 | 11 [symbol] | 0 | [symbol] 3 | 0 | 0 | 11 [symbol] |
| [symbol] | | | | | | | [symbol] | 0 | [symbol] 1 | 0 | [symbol] | 0 | [symbol] | 0 | 0 | [symbol] |
| [symbol] | | | | | | | | 0 | 53 | 0 | [symbol] | 0 | [symbol] | 0 | 0 | 415 |
| [symbol] | | | | | | | | 0 | 12 | 1 | 1 [symbol] 44 | 0 | 232 | 0 | 22 | [symbol] |
| [symbol] | | | | | | | | 0 | [symbol] | [symbol] 7 | [symbol] 1131 | 0 | [symbol] 10 | 0 | 0 | 1 [symbol] 7 |
| [symbol] | | | | | | | | 0 | 22 | 0 | 7 [symbol] | 0 | [symbol] 7 | 0 | 54 | [symbol] 4 |
| [symbol] | | | | | | | | 0 | [symbol] | 1 [symbol] | [symbol] | 0 | [symbol] 7 | 0 | 231 | 110 [symbol] |
| [symbol] | | | | | | | | 0 | [symbol] | 0 | [symbol] 153 | 0 | [symbol] 1 | 0 | 22 [symbol] | 11 [symbol] |
| [symbol] | | | | | | | | 0 | 0 | 0 | 4 [symbol] | 0 | 1 [symbol] | 0 | 72 | 10 [symbol] |
| [symbol] | | | | | | | | 0 | 170 | 0 | [symbol] | 0 | 17 [symbol] | 0 | 0 | 12 [symbol] |
| [symbol] | | | | | | | | 0 | 0 | [symbol] | [symbol] | 0 | [symbol] | 0 | 145 | 111 [symbol] |
| [symbol] | | | | | | | | 0 | 141 | [symbol] | 43120 | 0 | 7770 | 0 | 0 | [symbol] 22 |
| [symbol] -h | | 1 | | | | | | 0 | 230 | 2317 | [symbol] | 2311 | 2289 | 27300 | 540 | [symbol] 5271 |
| [symbol] -h | | 2 | | | | | | 143 | 1230 | 11829 | 2 [symbol] | [symbol] 77 | 8300 | 1 [symbol] | 962 | 13488 |
| [symbol] -h | | 3 | | | | | | 0 | 833 | 3254 | 4804 | 9945 | 4191 | [symbol] | 1113 | 4891 |
| [symbol] -h | | 4 | | | | | | 124 | 308 | 549 | 423 | 452 | 1541 | 1219 | 543 | 3061 |
| [symbol] -h | | 5 | | | | | | 4 [symbol] | 947 | 2451 | 1 [symbol] 4 | 2317 | 2005 | [symbol] | 745 | 12780 |
| [symbol] -h | | 6 | | | | | | 543 | 394 | 1775 | 11763 | 4300 | 4387 | 13 [symbol] 3 | 1111 | 111 [symbol] |
| [symbol] -h | | 7 | | | | | | 342 | 1137 | [symbol] | 66712 | 4422 | [symbol] 9 | 7 [symbol] | 1 [symbol] | 81304 |
| [symbol] -h | | 8 | | | | | | 815 | 531 | [symbol] 2 | [symbol] | 8917 | 4 [symbol] | 77711 | 1148 | 1 [symbol] |
| [symbol] -h | | 9 | | | | | | 0 | 5 [symbol] | [symbol] 71 | [symbol] | [symbol] | 7201 | 90138 | 1500 | 7000 |
| [symbol] -h | | 10 | | | | | | [symbol] | [symbol] | 7234 | 4384 | [symbol] | [symbol] 1 | [symbol] | 1725 | 24754 |
| [symbol] -h | | 11 | | | | | | 10 [symbol] | 1190 | [symbol] | [symbol] | [symbol] | 4324 | 34771 | 2075 | 4 [symbol] |
| [symbol] h | | 12 | | | | | | [symbol] | 1111 | 7 [symbol] | 7 [symbol] | 610 | [symbol] 72 | [symbol] | 1414 | [symbol] |
| [symbol] h | | 13 | | | | | | 246 | 1236 | [symbol] | 4142 | 1439 | 4384 | [symbol] | [symbol] | 11295 |
| [symbol] -h | | 14 | | | | | | [symbol] | [symbol] | 3624 | 77 [symbol] | [symbol] | [symbol] | [symbol] | 1120 | [symbol] |
| [symbol] -h | | 15 | | | | | | 1194 | [symbol] | 3315 | 13138 | [symbol] | [symbol] | [symbol] | 1630 | 87915 |
| [symbol] -h | | 16 | | | | | | 348 | [symbol] | [symbol] | [symbol] | [symbol] | [symbol] | 11314 | 1787 | 13457 |
| [symbol] -h | | 17 | | | | | | 32 | 708 | 3322 | 2344 | 11 [symbol] | 1451 | 41333 | 1114 | 37254 |
| [symbol] -h | | 18 | | | | | | 0 | 534 | 2431 | 8421 | [symbol] | [symbol] | 16871 | 1713 | 19614 |
| [symbol] -h | | 19 | | | | | | [symbol] | 344 | [symbol] | [symbol] | 4979 | 4912 | [symbol] | 1331 | 12378 |
| [symbol] -h | | 20 | | | | | | [symbol] | 264 | [symbol] | 4310 | 17 [symbol] | 544 | [symbol] | 1933 | [symbol] |
| [symbol] -h | | 21 | | | | | | 0 | 438 | 7 [symbol] 11 | [symbol] | [symbol] | 3116 | [symbol] | 1423 | 4737 |
| [symbol] -h | | 22 | | | | | | [symbol] | [symbol] | [symbol] | [symbol] | 3412 | 1391 | [symbol] | [symbol] | [symbol] |
| [symbol] -h | | 23 | | | | | | 0 | 182 | 5215 | [symbol] | 4125 | [symbol] | [symbol] | [symbol] | [symbol] |
| [symbol] -h | | 24 | | | | | | 0 | 443 | 2348 | 32224 | 1851 | 2148 | [symbol] | 1236 | [symbol] |
| [symbol] -h | | 25 | | | | | | [symbol] | [symbol] | [symbol] | 4231 | 29181 | [symbol] | [symbol] | [symbol] | [symbol] |
| [symbol] -h | | 27 | | | | | | 7 [symbol] | 414 | [symbol] | [symbol] | 11139 | 4787 | 11 [symbol] | 342 | 13111 |
| [symbol] | | 28 | | | | 28 | | 703 | 147 | 0 | [symbol] | 1182 | [symbol] | 4272 | 591 | 0 |
| [symbol] -h | | 28 | | | | | | 581 | [symbol] | 1320 | 47 [symbol] | 3198 | [symbol] | [symbol] | [symbol] | [symbol] |
| [symbol] | | 29 | | | | 30 | | 181 | 576 | 0 | 349 | 1033 | 3123 | [symbol] | 773 | 0 |
| [symbol] -h | | 31 | | | | | | 5 [symbol] | 740 | 43 [symbol] | [symbol] | 19717 | 20 [symbol] | 13172 | 1554 | [symbol] |
| [symbol] | | 32 | | | | | | 0 | 944 | 230 | 94 | 115 | 2044 | 1023 | 1192 | 2038 |
| [symbol] -h | | 33 | | | | | | 0 | [symbol] | 2916 | [symbol] | 3000 | 1347 | [symbol] | 1100 | [symbol] |
| [symbol] | | 34 | | | | | | 0 | 2 [symbol] | 0 | 454 | 0 | 2 [symbol] | 1110 | 805 | 0 |
| [symbol] -h | | 35 | | | | | | 0 | 451 | 0 | 312 | 222 | 1622 | [symbol] | 630 | 0 |
| [symbol] -h | | 36 | | | | | | 7 [symbol] | 473 | 443 | 381 | 11 | 1961 | [symbol] | [symbol] | 1375 |
| [symbol] -h | | 37 | | | | | | 0 | 375 | 1 [symbol] | 0 | 0 | 1441 | [symbol] | 720 | 0 |
| [symbol] -h | | 38 | | | | | | 0 | 842 | 0 | 363 | 0 | 3211 | [symbol] | 1054 | [symbol] |
| [symbol] -h | | 39 | | | | | | 0 | 338 | 1343 | 0 | 0 | 1194 | 3363 | [symbol] | 143 |
| [symbol] | | 40 | | | | | | 143 | 572 | 0 | 4 [symbol] | 342 | 2819 | 541 | 1218 | 0 |
| [symbol] -h | | 41 | | | | | | [symbol] | 294 | 518 | 138 | 11 | [symbol] | 0 | [symbol] | 0 |
| [symbol] -h | | 42 | | | | | | 629 | 429 | 0 | 573 | 464 | 1700 | 377 | 2223 | 0 |
| [symbol] -h | | 43 | | | | | | 0 | 421 | 154 | 0 | 14 [symbol] | [symbol] | 1341 | 1285 | 0 |
| [symbol] -h | | 44 | | | | | | [symbol] | 626 | 21 [symbol] | 425 | 3479 | 2371 | [symbol] | 1944 | 0 |
| [symbol] | | | | | [symbol] | | | 0 | 323 | 0 | 0 | 0 | 734 | 53 | 348 | 0 |
| [symbol] | | | | | 34 [symbol] | | | 0 | 0 | 2 [symbol] | 111 | 0 | 120 | 0 | 134 | 0 |
| [symbol] | | | | | 341 | | | 0 | 0 | 0 | [symbol] | 0 | 83 | [symbol] | 145 | 0 |
| [symbol] | | | | | 334 | 35 [symbol] | | 4165 | [symbol] | 34 [symbol] | 634 | 323 | 8377 | 294 | 3419 | [symbol] |
| [symbol] | | | | | 354 | 254 | | 77 [symbol] 5 | 2210 | 1298 | 300 | 50 | [symbol] | 182 | 1521 | [symbol] |
| [symbol] -h | | | | | 344 | | | 0 | 2 [symbol] | 82 | 225 | 35 | 297 | 64 | [symbol] | 0 |
| [symbol] | | | | | 342 | | | 0 | 0 | 32 | [symbol] | 0 | 245 | 0 | 452 | 254 |
| RPTBC | | | | | 234 | 234 | | 0 | 340 | 0 | 0 | 0 | 7 [symbol] | 0 | [symbol] | 0 |
| [symbol] -h | | | | | 332 | | | 0 | 0 | 11 | 19 | 24 | 91 | 45 | 836 | 0 |
| [symbol] | | | | | 330 | | | 80 | 91 | 0 | 47 | 0 | 71 | 0 | 637 | 0 |
| [symbol] -h | | | | | [symbol] | | | [symbol] | [symbol] | 0 | 1912 | 190 | [symbol] | 454 | 1432 | 7234 |
| [symbol] | | | | | 327 | | | 0 | [symbol] | 0 | 171 | 0 | 11$ | 411 | 429 | 0 |
| [symbol] | | | | | 328 | | | 309 | 4779 | 96 | 127 | 0 | [symbol] | 82 | 1824 | 9671 |
| [symbol] | | | | | 321 | | | 218 | 45 | 0 | 0 | 0 | 0 | 72 | 765 | 0 |
| [symbol] | | | | | 3 [symbol] | | | 0 | [symbol] | 0 | 283 | 238 | 2579 | 21 | 1311 | 0 |
| [symbol] -h | | | | | 318 | | | 2410 | 1322 | 724 | 2029 | 2188 | 4302 | 471 | 1067 | [symbol] |
| [symbol] -h | | | | | [symbol] | | | 812 | [symbol] | 0 | 1132 | [symbol] | 2543 | 0 | 701 | 1532 |
| [symbol] -h | | | | | 214 | | | 185 | 271 | 0 | 7 [symbol] | [symbol] | 2242 | 323 | 730 | 2522 |
| [symbol] -h | | | | | 211 | | | 0 | 0 | 0 | 183 | 47 | 53 | 251 | 1072 | 0 |
| [symbol] h | | | | | [symbol] | | | [symbol] | [symbol] | 47 | 136 | 242 | 100 | 147 | 462 | 0 |
| [symbol] -h | | | | | [symbol] | | | 306 | 610 | 154 | 494 | 89 | 1474 | 0 | 401 | 0 |
| [symbol] -h | | | | | 206 | | | 0 | 79 | 0 | 307 | 200 | 129 | [symbol] | 740 | 0 |
| [symbol] -h | | | | | [symbol] | | | 0 | 1575 | 0 | [symbol] | 675 | 1375 | 46 | 833 | 0 |
| [symbol] -h | | | | | 302 | | | 0 | 0 | 3944 | 123 | 72 | 208 | [symbol] | [symbol] | 228 |
| [symbol] -h | | | | | 2 [symbol] | | | 77 [symbol] | [symbol] | 21 [symbol] | [symbol] | 3723 | [symbol] | 1230 | 1487 | 4479 |
| [symbol] -h | | | | | [symbol] | | | 2006 | 4 [symbol] | 0 | [symbol] | [symbol] | 2775 | 0 | 2119 | 1673 |
| [symbol] -h | | | | | [symbol] | | | 0 | [symbol] | 0 | [symbol] | 815 | [symbol] | [symbol] 41 | 531 | 0 |
| [symbol] -h | | | | | [symbol] | | | 1237 | 1442 | 0 | 1966 | 799 | 2831 | 70 | 1713 | [symbol] |
| [symbol] -h | | | | | [symbol] | | | 0 | 0 | 0 | 1413 | 285 | 810 | 0 | [symbol] | [symbol] |
| [symbol] -h | | | | | 229 | | | 1424 | 482 | 0 | 3317 | [symbol] 5 | [symbol] 7 | 777 | [symbol] | 2243 |
| [symbol] -h | | | | | 279 | | | 22 [symbol] | 171 | 0 | 0 | 0 | 855 | 299 | 495 | 0 |
| [symbol] -h | | | | | 271 | | | 0 | 744 | 0 | 0 | 249 | 1362 | 374 | 944 | 0 |
| [symbol] | | | | | 276 | 275 | | 0 | 14 | 687 | 0 | 0 | 407 | 181 | 801 | 0 |
| [symbol] | | | | | [symbol] | | | 0 | 7 | 0 | [symbol] | 113 | 284 | 154 | 563 | 0 |
| [symbol] | | | | | [symbol] | | | 0 | 0 | 1147 | 161 | 0 | 0 | 245 | 738 | 752 |
| [symbol] 11 | | | | | 239 | 230 | | 0 | 300 | 222 | 357 | [symbol] | 531 | 2987 | 1400 | 0 |
| [symbol] 5 | | | | | 225 | 235 | | 0 | [symbol] | [symbol] | 127 | 40 | 0 | 481 | 751 | 0 |
| HT27 [symbol] | | | | | 234 | | | 79 | 1472 | 0 | 191 | 104 | 1276 | 0 | 1522 | 0 |
| [symbol] 7 | | | | | [symbol] | 223 | | 0 | 234 | 447 | 153 | 0 | 70 | 10 [symbol] | [symbol] 62 | 0 |
| [symbol] 8 | | | | | 231 | 231 | | 0 | 525 | 255 | 217 | 81 | 229 | 2538 | 817 | 0 |
| [symbol] 2 | | | | | 229 | 229 | | 314 | 815 | 0 | 12 | 25 | 994 | [symbol] | 719 | 0 |
| [symbol] 1 | | | | | 227 | 227 | | 194 | 287 | 0 | 0 | 2 | 82 | 1067 | [symbol] 47 | 0 |
| [symbol] -h | | | | | 222 | | | 0 | 57 | 0 | 0 | [symbol] | [symbol] | 48 | 453 | 0 |
| Heart-h | | | | | 215 | | | 0 | 406 | 0 | 0 | 0 | 3 [symbol] | 0 | 1007 | 0 |
| [symbol] -h | | | | | 214 | | | 0 | 299 | 291 | 183 | 230 | 141 | [symbol] | 976 | 0 |
| [symbol] -h | | | | | 213 | | | [symbol] | [symbol] | 147 | 0 | 57 | 0 | 152 | 1042 | 0 |
| [symbol] -h | | | | | 212 | | | 0 | 339 | 0 | 267 | 614 | 537 | 633 | [symbol] | 0 |
| HCAEC | | | | | 211 | 211 | | 0 | 67 | 0 | 0 | 99 | 0 | 254 | 793 | 0 |
| [symbol] -h | | | | | 219 | | | 0 | 179 | 0 | 2 [symbol] | 0 | 180 | [symbol] | 580 | 0 |
| [symbol] | | | | | 20 [symbol] | | | 0 | 317 | 0 | [symbol] | 0 | [symbol] | 0 | 731 | 0 |
| [symbol] -h | | | | | 20 [symbol] | | | 0 | 180 | 92 | 0 | 0 | 733 | 0 | 765 | 71 |
| [symbol] -h | | | | | 303 | | | 0 | 141 | 279 | [symbol] | 0 | 115 | 0 | 991 | 0 |
| [symbol] | | | | | 291 | | | 81 | 473 | 0 | 125 | 0 | [symbol] 44 | 0 | [symbol] | 0 |
| [symbol] -h | | | | | 189 | | | 95 | 104 | 0 | 111 | 140 | 122 | 0 | 72$ | 0 |
| [symbol] -h | | | | | 187 | | | 143 | 0 | 0 | 223 | 110 | 43 | 422 | 534 | 0 |
| [symbol] | | | | | 185 | | | 0 | 99 | 0 | 156 | 76 | [symbol] | 0 | 367 | 0 |
| [symbol] -h | | | | | 183 | | | 453 | 452 | 0 | 348 | 119 | 2341 | 109 | 806 | 0 |
| [symbol] | | | | | 179 | | | 0 | 178 | 0 | 0 | 78 | 286 | 0 | 371 | 0 |
| [symbol] -h | | | | | 81 | | | 0 | 573 | 0 | 0 | 189 | 1195 | 64 | [symbol] | 0 |
| [symbol] -h | | | | | [symbol] | | | 0 | 2301 | 0 | 778 | 700 | 2537 | [symbol] | [symbol] | 0 |
| [symbol] -h | | | | | [symbol] | | | 0 | [symbol] | 240 | 742 | 192 | [symbol] | 93 | [symbol] | 0 |
| [symbol] -h | | | | | [symbol] | | | 94 | 973 | 0 | 279 | 118 | [symbol] | 0 | 822 | 0 |
| [symbol] -h | | | | | 63 | | | 251 | 409 | [symbol] | [symbol] | 0 | 814 | 0 | 447 | 0 |
| [symbol] -h | | | | | 61 | | | 0 | 0 | 314 | 20 | 43 | 434 | 394 | 329 | 0 |
| [symbol] -h | | | | | [symbol] | | | 0 | 92 | 311 | 213 | 139 | 492 | 0 | 799 | 0 |
| [symbol] | | | | 78 | | | | 0 | [symbol] | 113 | 299 | 0 | 328 | 434 | 774 | 370 |
| [symbol] | | | | 81 | | | | 0 | 337 | 0 | 201 | 0 | [symbol] | 202 | 778 | 0 |
| [symbol] | | | | 83 | | | | 0 | 243 | 0 | 212 | 0 | 341 | 231 | 1374 | 0 |
| [symbol] | | | | [symbol] | | | | [symbol] | [symbol] | 0 | 256 | 51 | 473 | 0 | 649 | 0 |
| [symbol] | | | | [symbol] | | | | 294 | [symbol] | 0 | 449 | 75 | 5211 | 1347 | 2170 | 0 |
| [symbol] | | | | 92 | | | | 349 | 235 | 0 | 300 | 217 | 3141 | 170 | 1054 | [symbol] |
| [symbol] | | | | 92 | | | | 0 | 0 | 0 | 295 | 219 | [symbol] | 313 | 1114 | 23 |
| [symbol] | | | | 94 | | | | 0 | 125 | 0 | 123 | 11 | 0 | 199 | 1073 | 0 |
| [symbol] | | | | [symbol] | | | | [symbol] | 210 | [symbol] | 18 | 123 | 172 | 247 | 1399 | 0 |
| [symbol] | | | | 145 | | | | 540 | 11 | 240 | 330 | 1143 | 671 | 42 | 170 | 0 |
| [symbol] | | | | 145 | | | | 0 | 0 | 0 | 279 | 1314 | 165 | 54 | 431 | 0 |
| [symbol] | | | | [symbol] | | | | 372 | 194 | 104 | [symbol] | 1241 | 0 | 0 | 343 | 0 |
| [symbol] | | | | 132 | | | | 0 | 121 | 0 | 124 | 553 | 1020 | 91 | 230 | 243 |
| [symbol] | | | | 154 | | | | 0 | 342 | 0 | 200 | 327 | 791 | 327 | 340 | 0 |
| [symbol] | | | | [symbol] | | | | 0 | 443 | 299 | 173 | 619 | 830 | 0 | 182 | 0 |
| [symbol] | | | | [symbol] | | | | 0 | 47 | 0 | 371 | 342 | 0 | 0 | 742 | 4919 |
| [symbol] | | | | [symbol] | | | | 231 | 343 | 453 | 175 | 255 | 20 | 233 | [symbol] | 1977 |
| [symbol] | | | | 162 | | | | 349 | 0 | 0 | 0 | [symbol] | 332 | 0 | 304 | 0 |
| HCT [symbol] | | | | 1 [symbol] | | | | 337 | 203 | 344 | 83 | 102 | 0 | 0 | 199 | 0 |
| [symbol] | | | | 106 | | | | 0 | 377 | [symbol] | 131 | 1114 | 213 | 82 | 203 | 0 |
| T [symbol] | | | | [symbol] | | | | 230 | [symbol] | 21 | 45 | [symbol] | [symbol] | [symbol] | [symbol] | 0 |
| T [symbol] | | | | [symbol] | | | 0 | [symbol] | [symbol] | [symbol] | 104 | [symbol] | [symbol] | [symbol] | [symbol] | 0 |
| [symbol] | | | | 171 | | | | 0 | 3 | 120 | [symbol] | [symbol] | [symbol] | [symbol] | 530 | [symbol] |
| [symbol] | | | | 181 | | | | 31 | [symbol] | [symbol] | 1 | 81 | 247 | [symbol] | [symbol] | 0 |
| [symbol] | | | | 183 | | | | 0 | [symbol] | [symbol] | 0 | 123 | [symbol] | [symbol] | [symbol] | 0 |
| [symbol] | | | | 184 | | | | 0 | 0 | 203 | 270 | [symbol] | 237 | 0 | [symbol] | 0 |
| [symbol] | | | | 1 [symbol] | | | | 0 | 0 | 121 | 130 | 163 | [symbol] | 0 | [symbol] | 0 |
| [symbol] | | | | 1 [symbol] | | | | 118 | [symbol] | 0 | 87 | [symbol] | [symbol] | 0 | [symbol] | 0 |
| [symbol] | | | | 200 | | | | 227 | [symbol] | 0 | [symbol] | [symbol] | 1221 | 167 | [symbol] | 0 |
| [symbol] | | | | 202 | | | | 0 | 76 | 137 | 0 | 167 | 130 | 172 | [symbol] | 0 |
| [symbol] | | | | 204 | | | | [symbol] | [symbol] | [symbol] | 0 | 73 | [symbol] | [symbol] | 431 | 0 |
| [symbol] | | | | 205 | | | | 0 | 0 | [symbol] | 0 | [symbol] | [symbol] | 0 | [symbol] | 10 |
| [symbol] | | | | 206 | | | | 712 | 1711 | 0 | 351 | 360 | 1345 | 0 | 850 | 0 |
| [symbol] | | | | 216 | | | | 347 | 151 | [symbol] | 27 | [symbol] | 0 | 0 | [symbol] | 0 |
| [symbol] | | | | 218 | | | | 0 | [symbol] | 0 | 111 | 30 | 118 | 0 | [symbol] | 0 |
| [symbol] | | | | 220 | | | | 0 | 0 | 82 | 138 | 41 | 222 | 0 | [symbol] | 0 |
| [symbol] | | | | 221 | | | | 0 | 48 | 0 | 0 | 42 | 212 | [symbol] | [symbol] | 0 |
| [symbol] | | | | 223 | | | | 0 | 140 | 181 | 117 | 100 | 117 | 0 | 478 | [symbol] |
| [symbol] | | | | 225 | | | | 0 | 172 | 204 | 31 | 0 | 80 | 0 | 407 | 0 |
| [symbol] | | | | 241 | | | | 0 | 0 | 197 | 155 | [symbol] | [symbol] | [symbol] | [symbol] | 1088 |
| [symbol] | | | | 242 | | | | 10 | 0 | 173 | 304 | [symbol] | [symbol] | [symbol] | [symbol] | 0 |
| [symbol] | | | | 243 | | | | [symbol] | 202 | 10 | 370 | [symbol] | 720 | 370 | [symbol] | 0 |
| [symbol] | | | | 244 | | | | [symbol] | [symbol] | 0 | 372 | [symbol] | 330 | 84 | 10 | 817 |
| [symbol] | | | | 245 | | | | [symbol] | [symbol] | 834 | [symbol] | [symbol] | [symbol] | 0 | [symbol] | 0 |
| [symbol] | | | | 246 | | | | 0 | [symbol] | 847 | [symbol] | [symbol] | 0 | 0 | [symbol] | 0 |
| [symbol] | | | | 247 | | | | [symbol] | [symbol] | 0 | 312 | [symbol] | [symbol] | [symbol] | [symbol] | [symbol] |
| [symbol] | | | | 248 | | | | 0 | 122 | 0 | 229 | [symbol] | [symbol] | 0 | 425 | 0 |
| [symbol] | | | | 249 | | | | 0 | 0 | 1127 | 221 | [symbol] | [symbol] | 215 | [symbol] | [symbol] |
| [symbol] | | | | 250 | | | | 10 | 423 | 54 | 448 | [symbol] | [symbol] | [symbol] | [symbol] | [symbol] |
| [symbol] | | | | 251 | | | | 0 | [symbol] | 421 | 79 | [symbol] | [symbol] | 0 | [symbol] | 0 |
| [symbol] | | | | 252 | | | | [symbol] | 370 | 0 | 207 | 42 | 34 | 0 | 41 | 0 |
| [symbol] | | | | 253 | | | | 0 | 816 | 0 | 73 | [symbol] | [symbol] | 0 | [symbol] | [symbol] |
| [symbol] | | | | 254 | | | | [symbol] | [symbol] | [symbol] | 0 | [symbol] | [symbol] | [symbol] | [symbol] | 0 |
| [symbol] | | | | 255 | | | | [symbol] | 0 | 0 | 10 | [symbol] | [symbol] | 0 | [symbol] | 0 |
| [symbol] | | | | 256 | | | | [symbol] | [symbol] | 434 | 732 | [symbol] | [symbol] | [symbol] | [symbol] | 0 |
| [symbol] | | | | 257 | | | | 0 | [symbol] | [symbol] | 454 | [symbol] | [symbol] | [symbol] | [symbol] | 0 |
| [symbol] | | | | 258 | | | | 0 | [symbol] | 0 | 83 | 47 | [symbol] | 0 | [symbol] | 0 |
| [symbol] | | | | 259 | | | | 0 | [symbol] | 0 | 0 | [symbol] | [symbol] | [symbol] | [symbol] | 0 |
| [symbol] | | | | 260 | | | | 211 | 0 | 0 | 0 | [symbol] | [symbol] | 0 | [symbol] | 0 |
| [symbol] | | | | 261 | | | | 0 | [symbol] | 0 | 150 | [symbol] | [symbol] | 0 | [symbol] | 0 |
| [symbol] | | | | 262 | | | | 0 | [symbol] | 7839 | [symbol] | [symbol] | [symbol] | [symbol] | [symbol] | [symbol] |
| [symbol] | | | | 263 | | | | [symbol] | [symbol] | 780 | 287 | [symbol] | [symbol] | [symbol] | [symbol] | 0 |
| [symbol] | | | | 264 | | | | 0 | [symbol] | [symbol] | 70 | [symbol] | [symbol] | [symbol] | [symbol] | 0 |
| [symbol] | | | | 265 | | | | [symbol] | [symbol] | [symbol] | 70 | [symbol] | [symbol] | [symbol] | [symbol] | 0 |
| [symbol] | | | | 266 | | | | [symbol] | 0 | [symbol] | 2372 | [symbol] | [symbol] | [symbol] | [symbol] | 0 |
| [symbol] | | | | 267 | | | | 0 | 1211 | [symbol] | 128 | [symbol] | [symbol] | [symbol] | [symbol] | 400 |
| [symbol] | | | | 268 | | | | 0 | [symbol] | [symbol] | 41 | [symbol] | [symbol] | [symbol] | [symbol] | 0 |
| [symbol] | | | | 269 | | | | [symbol] | [symbol] | [symbol] | 0 | [symbol] | [symbol] | [symbol] | [symbol] | 1831 |
| [symbol] | | | | 270 | | | | 0 | 77 | 227 | 318 | [symbol] | [symbol] | [symbol] | [symbol] | 0 |
| [symbol] | | | | 271 | | | | 0 | 0 | 147 | 1711 | [symbol] | [symbol] | [symbol] | [symbol] | 0 |
| [symbol] | | | | 272 | | | | 0 | 0 | [symbol] | 219 | [symbol] | [symbol] | [symbol] | [symbol] | 0 |
| [symbol] | | | | [symbol] | | | | 0 | 1972 | 207 | 141 | [symbol] | [symbol] | [symbol] | [symbol] | 3088 |
| [symbol] | | | | [symbol] | | | | [symbol] | 0 | 0 | 123 | [symbol] | [symbol] | [symbol] | [symbol] | 0 |
| [symbol] | | | | [symbol] | | | | 0 | [symbol] | 0 | 222 | [symbol] | [symbol] | [symbol] | [symbol] | 4198 |
| [symbol] | | | | [symbol] | | | | [symbol] | [symbol] | 0 | 0 | [symbol] | [symbol] | [symbol] | [symbol] | 0 |
| [symbol] | | | | [symbol] | | | | 0 | [symbol] | 0 | 0 | [symbol] | [symbol] | [symbol] | [symbol] | 0 |
| [symbol] | | | | [symbol] | | | | 0 | [symbol] | 219 | [symbol] | [symbol] | [symbol] | [symbol] | [symbol] | 3114 |
| [symbol] | | | | [symbol] | | | | [symbol] | [symbol] | 0 | [symbol] | [symbol] | [symbol] | [symbol] | [symbol] | 2184 |
| [symbol] | | | | [symbol] | | | | [symbol] | [symbol] | 23 | 2318 | [symbol] | [symbol] | [symbol] | [symbol] | 0 |
| [symbol] | | | | [symbol] | | | | [symbol] | [symbol] | 0 | 232 | [symbol] | [symbol] | [symbol] | [symbol] | [symbol] |
| [symbol] | | | | [symbol] | | | | [symbol] | [symbol] | 0 | 842 | [symbol] | [symbol] | [symbol] | [symbol] | 0 |
| [symbol] | | | | [symbol] | | | | [symbol] | [symbol] | 280 | 44 | [symbol] | [symbol] | [symbol] | [symbol] | 0 |
| [symbol] | | | | [symbol] | | | | [symbol] | [symbol] | 0 | 20 | [symbol] | [symbol] | [symbol] | [symbol] | 0 |
| [symbol] | | | | [symbol] | | | | 0 | [symbol] | 100 | 0 | [symbol] | [symbol] | [symbol] | [symbol] | 0 |
| [symbol] | | | | [symbol] | | | | 0 | [symbol] | 0 | [symbol] | [symbol] | [symbol] | [symbol] | [symbol] | 0 |
| [symbol] | | | | [symbol] | | | | 0 | [symbol] | 41 | 0 | [symbol] | [symbol] | [symbol] | [symbol] | 0 |
| [symbol] | | | | [symbol] | | | | 0 | [symbol] | 0 | [symbol] | [symbol] | [symbol] | [symbol] | [symbol] | [symbol] |
| [symbol] | | | | [symbol] | | | | 0 | [symbol] | 0 | 0 | [symbol] | [symbol] | [symbol] | [symbol] | 0 |
| [symbol] | | | | [symbol] | | | | [symbol] | [symbol] | 628 | 277 | [symbol] | [symbol] | [symbol] | [symbol] | [symbol] |
| [symbol] | | | | [symbol] | | | | 0 | [symbol] | 183 | 434 | [symbol] | [symbol] | [symbol] | [symbol] | [symbol] |
| [symbol] | | | | [symbol] | | | | [symbol] | [symbol] | 0 | [symbol] | [symbol] | [symbol] | [symbol] | [symbol] | [symbol] |
| [symbol] | | | | [symbol] | | | | [symbol] | [symbol] | 0 | [symbol] | [symbol] | [symbol] | [symbol] | [symbol] | [symbol] |
| [symbol] | | | | [symbol] | | | | [symbol] | [symbol] | 0 | [symbol] | [symbol] | [symbol] | [symbol] | [symbol] | 0 |
| [symbol] | | | 50 | | | | | 0 | [symbol] | 0 | 0 | [symbol] | [symbol] | [symbol] | [symbol] | 0 |
| [symbol] | | | 52 | | | | | 0 | [symbol] | [symbol] | 237 | [symbol] | [symbol] | [symbol] | [symbol] | 0 |
| [symbol] | | | 54 | | | | | 0 | [symbol] | 0 | 0 | [symbol] | [symbol] | [symbol] | [symbol] | 0 |
| [symbol] | | | 55 | | | | | 0 | [symbol] | 130 | 37 | [symbol] | [symbol] | [symbol] | [symbol] | [symbol] |
| [symbol] | | | 56 | | | | | 0 | [symbol] | 0 | 0 | [symbol] | [symbol] | [symbol] | [symbol] | [symbol] |
| [symbol] | | | 60 | | | | | 0 | [symbol] | 0 | 47 | [symbol] | [symbol] | [symbol] | [symbol] | [symbol] |
| [symbol] | | | 62 | | | | | 0 | [symbol] | 0 | 412 | [symbol] | [symbol] | [symbol] | [symbol] | 364 |
| [symbol] | | | 63 | | | | | [symbol] | [symbol] | 0 | 0 | [symbol] | [symbol] | [symbol] | [symbol] | 0 |
| [symbol] | | | 64 | | | | | 0 | [symbol] | 18 | 234 | [symbol] | [symbol] | [symbol] | [symbol] | 0 |
| [symbol] | | | 65 | | | | | 0 | [symbol] | 0 | 183 | [symbol] | [symbol] | [symbol] | [symbol] | 0 |
| [symbol] | | | 66 | | | | | 0 | [symbol] | 83 | 34 | [symbol] | [symbol] | [symbol] | [symbol] | 0 |
| [symbol] | | | 67 | | | | | 0 | [symbol] | 0 | 294 | [symbol] | [symbol] | [symbol] | [symbol] | 0 |
| [symbol] | | | 68 | | | | | 0 | 0 | 0 | 178 | [symbol] | [symbol] | [symbol] | [symbol] | 0 |
| [symbol] | | | 69 | | | | | 0 | [symbol] | 0 | 77 | [symbol] | [symbol] | [symbol] | [symbol] | 0 |
| [symbol] | | | 70 | | | | | [symbol] | [symbol] | [symbol] | 20 | [symbol] | [symbol] | 0 | [symbol] | 37 |
| [symbol] | | | 71 | | | | | 0 | [symbol] | [symbol] | 474 | [symbol] | [symbol] | 0 | [symbol] | 0 |
| [symbol] | | | 72 | | | | | 0 | [symbol] | 0 | 230 | [symbol] | [symbol] | [symbol] | [symbol] | 0 |
| [symbol] | | | 73 | | | | | [symbol] | [symbol] | 234 | 187 | [symbol] | [symbol] | 0 | [symbol] | 381 |
| [symbol] | | | 74 | | | | | [symbol] | [symbol] | 0 | 234 | [symbol] | [symbol] | 0 | [symbol] | 0 |
| [symbol] | | | 76 | | | | | 0 | [symbol] | 0 | 222 | [symbol] | [symbol] | 0 | [symbol] | 0 |
| [symbol] | | | 77 | | | | | 0 | [symbol] | 0 | 730 | [symbol] | [symbol] | [symbol] | [symbol] | 0 |
| [symbol] | | | 79 | | | | | [symbol] | [symbol] | 0 | 308 | [symbol] | 470 | 0 | [symbol] | 277 |
| [symbol] | | | 80 | | | | | 0 | [symbol] | 0 | 483 | [symbol] | [symbol] | 0 | [symbol] | [symbol] |
| [symbol] | | | 82 | | | | | 0 | [symbol] | 200 | 112 | [symbol] | [symbol] | 0 | [symbol] | 0 |
| [symbol] | | | 85 | | | | | [symbol] | [symbol] | 0 | 128 | [symbol] | [symbol] | [symbol] | [symbol] | 0 |
| [symbol] | | | 87 | | | | | [symbol] | [symbol] | [symbol] | 20 | [symbol] | [symbol] | [symbol] | [symbol] | 0 |
| [symbol] | | | 170 | | | | | 0 | [symbol] | 0 | 158 | [symbol] | [symbol] | [symbol] | [symbol] | 0 |
| [symbol] | | | 189 | | | | | 0 | [symbol] | 0 | 0 | [symbol] | [symbol] | [symbol] | [symbol] | 0 |
| [symbol] | | | 187 | | | | | [symbol] | [symbol] | 0 | 12 | [symbol] | [symbol] | [symbol] | [symbol] | 0 |
| [symbol] | | | 188 | | | | | 0 | 1097 | 0 | 82 | [symbol] | [symbol] | [symbol] | [symbol] | 0 |
| [symbol] | | | 191 | | | | | 0 | 277 | 0 | 195 | [symbol] | [symbol] | [symbol] | [symbol] | 0 |
| [symbol] | | | 207 | | | | | 0 | 281 | 7 | 187 | [symbol] | [symbol] | [symbol] | [symbol] | 182 |
| [symbol] | | | 216 | | | | | 0 | 0 | 0 | 100 | [symbol] | [symbol] | [symbol] | [symbol] | 138 |
| [symbol] | | | 217 | | | | | [symbol] | 0 | 0 | 43 | [symbol] | [symbol] | [symbol] | [symbol] | 0 |
| [symbol] | | | 224 | | | | | 0 | 0 | 0 | 275 | [symbol] | [symbol] | [symbol] | [symbol] | 0 |
| [symbol] | | | 226 | | | | | 0 | 1088 | 173 | 294 | [symbol] | [symbol] | [symbol] | [symbol] | 0 |
| [symbol] | | | 228 | | | | | [symbol] | 218 | 0 | 0 | [symbol] | [symbol] | [symbol] | [symbol] | 0 |
| [symbol] | | | 230 | | | | | 0 | 247 | 0 | [symbol] | [symbol] | [symbol] | [symbol] | [symbol] | 0 |
| [symbol] | | | [symbol] | | | | | 0 | 172 | 0 | [symbol] | [symbol] | [symbol] | [symbol] | [symbol] | [symbol] |
| [symbol] | | | [symbol] | | | | | 0 | 71 | 283 | [symbol] | [symbol] | [symbol] | [symbol] | [symbol] | [symbol] |
| [symbol] | | | [symbol] | | | | | 0 | 38 | 0 | [symbol] | [symbol] | [symbol] | [symbol] | [symbol] | 0 |
| [symbol] | | | [symbol] | | | | | 0 | 1248 | 930 | [symbol] | [symbol] | [symbol] | [symbol] | [symbol] | 0 |
| [symbol] | | | [symbol] | | | | | 0 | 2033 | 0 | [symbol] | [symbol] | [symbol] | [symbol] | [symbol] | 0 |
| [symbol] | | | [symbol] | | | | | 0 | 44 | 0 | [symbol] | [symbol] | [symbol] | [symbol] | [symbol] | 0 |
| [symbol] | | | [symbol] | | | | | [symbol] | 0 | 0 | [symbol] | [symbol] | [symbol] | [symbol] | [symbol] | 0 |
| [symbol] | | | [symbol] | | | | | 0 | 98 | 0 | [symbol] | [symbol] | [symbol] | [symbol] | [symbol] | 0 |
| [symbol] | | | 318 | | | | | 0 | 0 | 0 | [symbol] | [symbol] | [symbol] | [symbol] | [symbol] | 0 |
| [symbol] | | | 328 | | | | | [symbol] | 0 | 0 | [symbol] | [symbol] | [symbol] | [symbol] | [symbol] | 0 |
| [symbol] | | | 283 | | | | | 0 | 0 | 0 | [symbol] | [symbol] | [symbol] | [symbol] | [symbol] | 0 |
| [symbol] | 183 | | | | | | | 0 | 177 | 245 | [symbol] | 1180 | 2222 | [symbol] | [symbol] | 1143 |
| [symbol] | 181 | | | | | | | [symbol] | 208 | 971 | 825 | 914 | 9749 | [symbol] | 728 | 8209 |
| [symbol] | 188 | | | | | | | [symbol] | 278 | 1184 | 1441 | 838 | [symbol] | 4437 | 878 | 2909 |
| [symbol] | 187 | | | | | | | 11137 | 2218 | 0 | 1828 | 1894 | 28341 | 2387 | 1838 | 384 |
| [symbol] | 139 | | | | | | | [symbol] | 943 | 9 | 1799 | 2088 | 1204 | [symbol] | 005 | 14293 |
| [symbol] | 149 | | | | | | | 491 | 517 | 1877 | 991 | [symbol] | 10627 | 8344 | [symbol] | 9 |
| [symbol] | 148 | | | | | | | 9433 | 1256 | 140 | [symbol] | [symbol] | 28534 | 2044 | 1309 | 2239 |
| M14 | 147 | | | | | | | 3031 | 9 | 489 | 2041 | 1883 | 3478 | 1300 | 1035 | 6 |
| [symbol] | 146 | | | | | | | 4423 | 304 | 1299 | 2779 | 2137 | 10063 | [symbol] | 639 | 607 |
| [symbol] | 145 | | | | | | | 2545 | 157 | 726 | 754 | 1215 | 4493 | 2428 | 594 | 430 |
| [symbol] | 144 | | | | | | | [symbol] | 83 | 3004 | 1173 | 721 | 3000 | 1531 | 805 | 2911 |
| [symbol] | 143 | | | | | | | 5224 | 487 | 3887 | 884 | 442 | 3000 | 1020 | 1532 | 11887 |
| [symbol] | 142 | | | | | | | 338 | 8 | 9004 | 2781 | 2618 | 18384 | 2004 | 891 | 3004 |
| [symbol] | 141 | | | | | | | 291 | 218 | 0 | 499 | 0 | 3003 | 379 | 442 | 8 |
| [symbol] | 140 | | | | | | | 2914 | 195 | 1344 | 1741 | 476 | 19872 | 1717 | 919 | 3811 |
| [symbol] | 139 | | | | | | | 4662 | [symbol] | 936 | [symbol] | 288 | [symbol] | 882 | 879 | 1620 |
| [symbol] | 138 | | | | | | | 1288 | 48 | 995 | 2291 | 1148 | 7979 | 2354 | 943 | 2000 |
| [symbol] | 137 | | | | | | | 3599 | 191 | 1883 | [symbol] | 1417 | 11189 | 231 | 1214 | 1103 |
| [symbol] | 136 | | | | | | | 2534 | 52 | 1973 | 1824 | 977 | 20902 | 2457 | 887 | 1006 |
| [symbol] | 135 | | | | | | | 4391 | 443 | 1305 | 1150 | 838 | 1214 | 571 | 830 | 1744 |
| [symbol] | 134 | | | | | | | 4889 | 318 | 1843 | [symbol] | 404 | [symbol] | 2003 | 821 | 1147 |
| [symbol] | 133 | | | | | | | 1724 | 294 | 979 | 1634 | 323 | [symbol] | 244 | 704 | 0 |
| [symbol] | 132 | | | | | | | 2284 | 187 | 1803 | 829 | 742 | 11009 | 2314 | 722 | 0 |
| [symbol] | 131 | | | | | | | 2664 | 29 | 0 | 1239 | 719 | 5239 | 858 | 904 | 0 |
| [symbol] | 130 | | | | | | | 2297 | 486 | 2066 | 1079 | 834 | [symbol] | [symbol] | 952 | 0 |
| [symbol] | 129 | | | | | | | 2213 | 370 | 0 | 719 | [symbol] | 13004 | 2500 | 843 | 0 |
| [symbol] | 128 | | | | | | | 914 | 317 | 8314 | 911 | 640 | 2071 | 7442 | 534 | [symbol] |
| [symbol] | 127 | | | | | | | 1412 | 63 | 621 | 903 | 461 | 3231 | 1331 | [symbol] | 312 |
| [symbol] | 126 | | | | | | | 2183 | 41 | 0 | 1330 | 634 | [symbol] | 3124 | 902 | [symbol] |
| [symbol] | 125 | | | | | | | 3471 | 320 | 0 | 841 | 330 | [symbol] | 1183 | 345 | [symbol] |
| [symbol] | 124 | | | | | | | 5491 | 0 | 1125 | 1997 | 200 | 2993 | [symbol] | 634 | [symbol] |
| [symbol] | 123 | | | | | | | 1951 | 45 | 44 | 234 | 244 | 1934 | 0 | 843 | [symbol] |
| [symbol] | 122 | | | | | | | 2006 | 184 | 809 | 178 | 1623 | 11709 | 11036 | 634 | 827 |
| [symbol] | 121 | | | | | | | 839 | 0 | 0 | 204 | 543 | [symbol] | 305 | 634 | 0 |
| [symbol] | 120 | | | | | | | 5443 | 0 | 1010 | 823 | 1107 | 4271 | 783 | [symbol] | 0 |
| [symbol] | 119 | | | | | | | 1503 | 119 | 1759 | [symbol] | 2414 | [symbol] | [symbol] | 934 | 9425 |
| [symbol] | 118 | | | | | | | 3767 | 0 | 0 | 830 | 252 | 6341 | 1000 | 100 | 200 |
| [symbol] | 117 | | | | | | | 1254 | 204 | 0 | 1027 | 2229 | 11490 | 3863 | 1723 | 857 |
| [symbol] | 116 | | | | | | | 8623 | 44 | 1399 | 2372 | 1624 | 24402 | 2314 | 99 | 1344 |
| [symbol] | 115 | | | | | | | 2000 | 150 | 8853 | 834 | 1592 | 12134 | 1242 | 942 | 0 |
| [symbol] | 114 | | | | | | | 2627 | 457 | 10 | 717 | 367 | [symbol] | 833 | 842 | 1466 |
| [symbol] | 113 | | | | | | | 456 | 0 | 1659 | 873 | 284 | [symbol] | [symbol] | 282 | 1027 |
| [symbol] | 112 | | | | | | | 2617 | 634 | 75 | 1118 | 401 | 1774 | 1809 | 941 | 0 |
| [symbol] | 111 | | | | | | | 1007 | 909 | 1723 | 2304 | 1761 | 20238 | 3231 | 1074 | 751 |
| [symbol] | 110 | | | | | | | 2254 | 204 | 9443 | 883 | 1814 | 11839 | 4509 | 843 | [symbol] |
| [symbol] | 109 | | | | | | | 1204 | 0 | 1175 | 1767 | 1721 | 7279 | [symbol] | [symbol] | 1034 |
| [symbol] | 108 | | | | | | | 2209 | 203 | 729 | 877 | 739 | 8851 | 2516 | 524 | 2200 |
| [symbol] | 107 | | | | | | | 2274 | 449 | 1277 | 934 | 934 | 17829 | 1884 | 476 | 0 |
| [symbol] | 106 | | | | | | | 1144 | 228 | 0 | 495 | 200 | 4322 | 1334 | 722 | 0 |
| [symbol] | 105 | | | | | | | [symbol] | [symbol] | 0 | 3523 | 1047 | 20447 | 209 | 549 | 321 |
| [symbol] | 104 | | | | | | | 2000 | 318 | 0 | [symbol] | [symbol] | [symbol] | 2106 | 412 | 1904 |
| [symbol] | 103 | | | | | | | [symbol] | 126 | 0 | [symbol] | [symbol] | [symbol] | 1852 | 1001 | 0 |
| [symbol] | 102 | | | | | | | [symbol] | 419 | 0 | 1091 | 816 | [symbol] | [symbol] | [symbol] | 3219 |
| [symbol] | 101 | | | | | | | [symbol] | 311 | 0 | 1162 | 911 | 7912 | 446 | 300 | 0 |
| [symbol] | 100 | | | | | | | 212 | 0 | 0 | [symbol] | [symbol] | [symbol] | 0 | [symbol] | 0 |
| [symbol] | 99 | | | | | | | [symbol] | 170 | 77 | 1733 | 915 | [symbol] | [symbol] | [symbol] | [symbol] |
| [symbol] | 98 | | | | | | | [symbol] | 0 | 477 | 422 | 160 | 7004 | 376 | [symbol] | 0 |
| [symbol] | 97 | | | | | | | 3307 | 194 | 0 | [symbol] | [symbol] | [symbol] | 1410 | [symbol] | 401 |
| [symbol] | 44 | | | | | | | 334 | 94 | 14 | [symbol] | 21 | 1718 | [symbol] | 1434 | 0 |
| [symbol] | 47 | | | | | | | [symbol] | 15 | [symbol] | 231 | 0 | [symbol] | [symbol] | 1342 | 0 |
| [symbol] | 44 | | | | | | | 834 | [symbol] | 0 | 183 | 82 | [symbol] | 9715 | 1438 | [symbol] |
| [symbol] | 24 | | | | | | | [symbol] | 737 | [symbol] | 402 | [symbol] | 2737 | [symbol] | 34 | 1742 |
| [symbol] -1 | | | | | | | wt | 0 | 0 | 0 | 8421 | [symbol] | 0 | [symbol] | [symbol] | 0 |
| [symbol] -3 | | | | | | | wt | 0 | 0 | 0 | 4212 | [symbol] | 0 | [symbol] | [symbol] | 0 |
| [symbol] -4 | | | | | | | wt | 0 | 0 | 0 | 8323 | [symbol] | 0 | [symbol] | [symbol] | 0 |
| [symbol] -5 | | | | | | | wt | 0 | 0 | 0 | [symbol] | 210 | 0 | [symbol] | [symbol] | 0 |
| [symbol] -7 | | | | | | | [symbol] | 0 | 0 | 0 | 192 | 118 | 0 | [symbol] | [symbol] | 0 |
| EXVX-1 | | | | | | | [symbol] | 0 | 0 | 0 | 1343 | 0 | 0 | [symbol] | [symbol] | 0 |
| EXVX-4 | | | | | | | [symbol] | 0 | 0 | 0 | [symbol] | 11 | 0 | [symbol] | [symbol] | 0 |
| EXVX-5 | | | | | | | [symbol] | 0 | 0 | 0 | [symbol] | 0 | 0 | [symbol] | [symbol] | 0 |
| EXVX-6 | | | | | | | [symbol] | 0 | 0 | 0 | [symbol] | [symbol] | 0 | [symbol] | [symbol] | 0 |
| EXVX-7 | | | | | | | wt | 0 | 0 | 0 | [symbol] | [symbol] | 0 | [symbol] | [symbol] | 0 |
| MCF-7-1 | | | | | | | wt | 0 | 0 | 0 | 718 | [symbol] | 0 | [symbol] | [symbol] | 0 |
| MCF-7-3 | | | | | | | wt | 0 | 0 | 0 | [symbol] | [symbol] | 0 | [symbol] | [symbol] | 0 |
| MCF-7-4 | | | | | | | wt | 0 | 0 | 0 | 1135 | [symbol] | 0 | [symbol] | [symbol] | 0 |
| MCF-7-6 | | | | | | | wt | 0 | 0 | 0 | [symbol] | [symbol] | 0 | [symbol] | [symbol] | 0 |
| MCF-7-7 | | | | | | | [symbol] | 0 | 0 | 0 | 1337 | [symbol] | 0 | [symbol] | [symbol] | 0 |
| ADR-RES-1 | | | | | | | [symbol] | 0 | 0 | 0 | [symbol] | 0 | 0 | [symbol] | [symbol] | 0 |
| ADR-RES-3 | | | | | | | [symbol] | 0 | 0 | 0 | 920 | [symbol] | 0 | [symbol] | [symbol] | 0 |
| ADR-RES-4 | | | | | | | [symbol] | 0 | 0 | 0 | [symbol] | 0 | 0 | [symbol] | [symbol] | 0 |
| ADR-RES-6 | | | | | | | [symbol] | 0 | 0 | 0 | 472 | 112 | 0 | [symbol] | [symbol] | 0 |
| ADR-RES-7 | | | | | | | wt | 0 | 0 | 0 | [symbol] | 0 | 0 | [symbol] | [symbol] | 0 |
| WI 30-1 | | | | | | | wt | 0 | 0 | 0 | 0 | 222 | 0 | [symbol] | [symbol] | 0 |
| WI 30-3 | | | | | | | wt | 0 | 0 | 0 | [symbol] | 172 | 0 | [symbol] | [symbol] | 0 |
| WI 30-4 | | | | | | | wt | 0 | 0 | 0 | 751 | [symbol] | 0 | [symbol] | [symbol] | 0 |
| WI 30-6 | | | | | | | wt | 0 | 0 | 0 | [symbol] | [symbol] | 0 | [symbol] | [symbol] | 0 |
| WI 30-7 | | | | | | | [symbol] | 0 | 0 | 0 | [symbol] | 0 | 0 | [symbol] | [symbol] | 0 |
| [symbol] | | | | | | | [symbol] | 0 | 0 | 0 | [symbol] | [symbol] | 0 | [symbol] | [symbol] | 0 |
| [symbol] | | | | | | | [symbol] | 0 | 0 | 0 | [symbol] | [symbol] | 0 | [symbol] | [symbol] | 0 |
| [symbol] | | | | | | | [symbol] | 0 | 0 | 0 | [symbol] | [symbol] | 0 | [symbol] | [symbol] | 0 |
| [symbol] | | | | | | | [symbol] | 0 | 0 | 0 | [symbol] | [symbol] | 0 | [symbol] | [symbol] | 0 |
| [symbol] | | | | | | | [symbol] | 0 | 0 | 0 | [symbol] | [symbol] | 0 | [symbol] | [symbol] | 0 |
| [symbol] -1 | | | | | | | [symbol] | 0 | 0 | 0 | 0 | 0 | 0 | [symbol] | [symbol] | 0 |
| [symbol] -3 | | | | | | | [symbol] | 0 | 0 | 0 | 0 | 0 | 0 | [symbol] | [symbol] | 0 |
| [symbol] -4 | | | | | | | [symbol] | 0 | 0 | 0 | 2070 | [symbol] | 0 | [symbol] | [symbol] | 0 |
| [symbol] -5 | | | | | | | [symbol] | 0 | 0 | 0 | 1400 | [symbol] | 0 | [symbol] | [symbol] | 0 |
| [symbol] -7 | | | | | | | wt | 0 | 0 | 0 | 0 | 0 | 0 | [symbol] | [symbol] | 0 |
| [symbol] -3 | | | | | | | [symbol] | 0 | 0 | 0 | 4710 | 141 | 0 | [symbol] | [symbol] | 0 |
| [symbol] -2 | | | | | | | wt | 0 | 0 | 0 | 2003 | [symbol] | 0 | [symbol] | [symbol] | 0 |
| HCT-116-1 | | | | | | | wt | 0 | 0 | 0 | [symbol] | 143 | 0 | [symbol] | [symbol] | 0 |
| HCT-116-2 | | | | | | | [symbol] | 0 | 0 | 0 | [symbol] | 44 | 0 | [symbol] | [symbol] | 0 |
| [symbol] -2 | | | | | | | wt | 0 | 0 | 0 | [symbol] | [symbol] | 0 | [symbol] | [symbol] | 0 |
| [symbol] -1 | | | | | | | wt | 0 | 0 | 0 | [symbol] | [symbol] | 0 | [symbol] | [symbol] | 0 |
| [symbol] -2 | | | | | | | [symbol] | 0 | 0 | 0 | [symbol] | 10 | 0 | [symbol] | [symbol] | 0 |
| [symbol] -1 | | | | | | | [symbol] | 0 | 0 | 0 | 317 | 2004 | 0 | [symbol] | [symbol] | 0 |
| [symbol] -2 | | | | | | | wt | 0 | 0 | 0 | 850 | [symbol] | 0 | [symbol] | [symbol] | 0 |
| OVCAR-4-1 | | | | | | | wt | 0 | 0 | 0 | 89 | [symbol] | 0 | [symbol] | [symbol] | 0 |
| OVCAR-4-2 | | | | | | | [symbol] | 0 | 0 | 0 | 1843 | [symbol] | 0 | [symbol] | [symbol] | 0 |
| OVCAR-4-1 | | | | | | | [symbol] | 0 | 0 | 0 | [symbol] | [symbol] | 0 | [symbol] | [symbol] | 0 |
| OVCAR-4-2 | | | | | | | wt | 0 | 0 | 0 | 10 | [symbol] | 0 | [symbol] | [symbol] | 0 |
| [symbol] -2 | | | | | | | [symbol] | 0 | 0 | 0 | 1433 | [symbol] | 0 | [symbol] | [symbol] | 0 |
| [symbol] -2 | | | | | | | [symbol] | 0 | 0 | 0 | 731 | [symbol] | 0 | [symbol] | [symbol] | 0 |
| [symbol] -2 | | | | | | | [symbol] | 0 | 0 | 0 | 0 | [symbol] | 0 | [symbol] | [symbol] | 0 |
| [symbol] -1 | | | | | | | [symbol] | 0 | 0 | 0 | 0 | [symbol] | 0 | [symbol] | [symbol] | 0 |
| [symbol] -2 | | | | | | | [symbol] | 0 | 0 | 0 | [symbol] | [symbol] | 0 | [symbol] | [symbol] | 0 |
| [symbol] -2 | | | | | | | [symbol] | 0 | 0 | 0 | [symbol] | [symbol] | 0 | [symbol] | [symbol] | 0 |
| [symbol] -1 | | | | | | | [symbol] | 0 | 0 | 0 | [symbol] | 0 | 0 | [symbol] | [symbol] | 0 |
| [symbol] -2 | | | | | | | [symbol] | 0 | 0 | 0 | 1155 | 1 | 0 | [symbol] | [symbol] | 0 |
| [symbol] -1 | | | | | | | [symbol] | 0 | 0 | 0 | [symbol] | [symbol] | 0 | [symbol] | [symbol] | 0 |
| [symbol] -2 | | | | | | | wt | 0 | 0 | 0 | 0 | [symbol] | 0 | [symbol] | [symbol] | 0 |
| [symbol] -1 | | | | | | | wt | 0 | 0 | 0 | [symbol] | [symbol] | 0 | [symbol] | [symbol] | 0 |
| [symbol] -2 | | | | | | | wt | 0 | 0 | 0 | [symbol] | 0 | 0 | [symbol] | [symbol] | 0 |
| WI 30-2 | | | | | | | | 0 | 0 | 0 | [symbol] | 0 | 0 | [symbol] | [symbol] | 0 |
| [symbol] -1 | | | | | | | | 0 | 0 | 0 | [symbol] | [symbol] | 0 | [symbol] | [symbol] | 0 |
| [symbol] -2 | | | | | | | | 0 | 0 | 0 | [symbol] | [symbol] | 0 | [symbol] | [symbol] | 0 |
| [symbol] -3 | | | | | | | | 0 | 0 | 0 | [symbol] | [symbol] | 0 | [symbol] | [symbol] | 0 |
| [symbol] -4 | | | | | | | | 0 | 0 | 0 | 0 | 143 | 0 | [symbol] | [symbol] | 0 |
| [symbol] -5 | | | | | | | | 0 | 0 | 0 | 0 | [symbol] | 0 | [symbol] | [symbol] | 0 |
| [symbol] -6 | | | | | | | | 0 | 0 | 0 | [symbol] | 1 | 0 | [symbol] | [symbol] | 0 |
| [symbol] -7 | | | | | | | | 0 | 0 | 0 | [symbol] | [symbol] | 0 | [symbol] | [symbol] | 0 |
| [symbol] -8 | | | | | | | | 0 | 0 | 0 | 0 | [symbol] | 0 | [symbol] | 1234 | 0 |
| [symbol] -7 | | | | | | | | 0 | 0 | 0 | 4153 | 258 | [symbol] | 11 [symbol] | [symbol] | 0 |
| [symbol] -8 | | | | | | | | 0 | 0 | 0 | 0 | 72 | 0 | 1134 | [symbol] | 0 |
| [symbol] | | | | | | | | 0 | 0 | 0 | 352 | 17 | 0 | [symbol] | [symbol] | 0 |
| [symbol] 11 | | | | | | | | 0 | 0 | 0 | 2910 | 477 | 0 | 1191 | 732 | 0 |
| [symbol] 12 | | | | | | | | 0 | 0 | 0 | 0 | [symbol] | 0 | [symbol] | [symbol] | 0 |
| [symbol] | | | | | | | | 0 | 0 | 0 | 11437 | 102 | 0 | 910 | 582 | 0 |
| [symbol] 1 | | | | | | | | 0 | 0 | 0 | 0 | [symbol] | 0 | [symbol] | 434 | 0 |
| [symbol] 2 | | | | | | | | 0 | 0 | 0 | 282 | 0 | 0 | [symbol] | [symbol] | 0 |
| [symbol] 3 | | | | | | | | 0 | 0 | 0 | 10 [symbol] | [symbol] | [symbol] | 337 | [symbol] | 0 |
| [symbol] 4 | | | | | | | | 0 | 0 | 0 | 3675 | 67 | 0 | [symbol] | [symbol] | 0 |
| [symbol] 5 | | | | | | | | 0 | 0 | 0 | 217 | 72 | 0 | [symbol] | [symbol] | 0 |
| [symbol] 6 | | | | | | | [symbol] | 0 | 0 | 0 | 0 | 40 | 0 | 62 | [symbol] | 0 |
| [symbol] -6 | | | | | | | [symbol] | 0 | 0 | 0 | [symbol] | 79 | 0 | [symbol] | [symbol] | 0 |
| EKYX-6 | | | | | | | [symbol] | 0 | 0 | 0 | [symbol] | [symbol] | 0 | [symbol] | [symbol] | 0 |
| HCT-114-7 | | | | | | | [symbol] | 0 | 0 | 0 | 0 | [symbol] | 0 | [symbol] | [symbol] | 0 |
| HCT-116-8 | | | | | | | [symbol] | 0 | 0 | 0 | [symbol] | 122 | 0 | 175 | 875 | 0 |
| HT29-1 | | | | | | | [symbol] | 0 | 0 | 0 | 3521 | 0 | 0 | [symbol] | [symbol] | 0 |
| HT29-7 | | | | | | | [symbol] | 0 | 0 | 0 | 3147 | 20 | 0 | [symbol] | [symbol] | 0 |
| HT29-8 | | | | | | | [symbol] | 0 | 0 | 0 | 467 | 0 | 0 | 200 | [symbol] | 0 |
| [symbol] -7 | | | | | | | [symbol] | 0 | 0 | 0 | 375 | [symbol] | 0 | 21 | [symbol] | 0 |
| [symbol] -8 | | | | | | | [symbol] | 0 | 0 | 0 | 0 | [symbol] | 0 | 21 | [symbol] | 0 |
| [symbol] -7 | | | | | | | [symbol] | 0 | 0 | 0 | 440 | [symbol] | 0 | [symbol] | [symbol] | 0 |
| [symbol] -8 | | | | | | | [symbol] | 0 | 0 | 0 | 0 | [symbol] | 0 | [symbol] | [symbol] | 0 |
| OVCAR4-7 | | | | | | | [symbol] | 0 | 0 | 0 | 1305 | 423 | 0 | 0 | 934 | 0 |
| OVCAR4-8 | | | | | | | [symbol] | 0 | 0 | 0 | 127 | [symbol] | 0 | 2777 | [symbol] | 0 |
| OVCAR4-7 | | | | | | | [symbol] | 0 | 0 | 0 | 325 | 0 | 0 | [symbol] | [symbol] | 0 |
| OVCAR4-8 | | | | | | | [symbol] | 0 | 0 | 0 | 0 | 315 | 0 | [symbol] | [symbol] | 0 |
| MCF-8-8 | | | | | | | [symbol] | 0 | 0 | 0 | 740 | [symbol] | 0 | [symbol] | [symbol] | 0 |
| [symbol] | | | | | | | [symbol] | 0 | 0 | 0 | 3013 | 0 | 0 | [symbol] | [symbol] | 0 |
| HeLA-8 | | | | | | | [symbol] | 0 | 0 | 0 | 2243 | 117 | 0 | [symbol] | [symbol] | 0 |
| SW480-7 | | | | | | | [symbol] | 0 | 0 | 0 | 1081 | 452 | 0 | 0 | 1024 | 0 |
| SW480-8 | | | | | | | [symbol] | 0 | 0 | 0 | 7328 | 417 | 0 | 302 | 712 | 0 |
| H1290-6 | | | | | | | [symbol] | 0 | 0 | 0 | 2343 | 0 | 0 | 0 | 737 | 0 |
| [symbol] -7 | | | | | | | [symbol] | 0 | 0 | 0 | 3128 | 0 | 0 | [symbol] | [symbol] | 0 |
| [symbol] -8 | | | | | | | [symbol] | 0 | 0 | 0 | 1013 | 0 | 0 | [symbol] | [symbol] | 0 |
| [symbol] -7 | | | | | | | [symbol] | 0 | 0 | 0 | 2134 | 0 | 0 | 13 | 951 | 0 |
| [symbol] -8 | | | | | | | [symbol] | 0 | 0 | 0 | 0 | [symbol] | 0 | [symbol] | [symbol] | 0 |
| [symbol] -7 | | | | | | | [symbol] | 0 | 0 | 0 | 4024 | [symbol] | 0 | 171 | 615 | 0 |
| [symbol] -8 | | | | | | | [symbol] | 0 | 0 | 0 | 4437 | 0 | 0 | 301 | [symbol] | 0 |
| [symbol] | | | | | | | | 0 | 2 [symbol] | 0 | 47 | [symbol] | [symbol] | [symbol] | [symbol] | 0 |
| [symbol] RNA | | | | | | | | 0 | [symbol] | 0 | 304 | 1454 | 1335 | 1421 | [symbol] | 0 |
| CRL 1372 3/17/ [symbol] | | | | | | | | 0 | 104 | 154 | 32 | 0 | 42 | 445 | 336 | 1013 |
| [symbol] | | | | | | 54 | | 274 | 434 | 0 | 25 | 153 | 572 | 2372 | 1228 | [symbol] |
| HT368 | | | | | | | | 1326 | 1327 | 0 | 217 | 70 | 5103 | 0 | [symbol] | 0 |
| HT378 | | | | | | | | 20 | 104 | 0 | 163 | 65 | 723 | 0 | [symbol] | 0 |
| HT386 | | | | | | | | [symbol] | [symbol] | 0 | [symbol] | [symbol] | [symbol] | 375 | 1937 | [symbol] |
| HT308 | | | | | | | | 0 | 234 | 0 | 449 | 0 | 494 | 95 | 2033 | [symbol] |
| [symbol] -3 | | | | | | 173 | | 0 | 0 | [symbol] | 34 | 115 | 0 | 0 | [symbol] | 0 |
| [symbol] -5 | | | | | | 176 | | 0 | 335 | 790 | 0 | 0 | 10 | 101 | 345 | 0 |
| [symbol] | | | | | | 177 | | 576 | 345 | 241 | 104 | 0 | [symbol] | 115 | [symbol] | 0 |
| [symbol] | | | | | | | | 0 | 1129 | 325 | 0 | 0 | [symbol] | [symbol] | [symbol] | 0 |
| [symbol] -10 | | | | | | 217 | | 0 | 0 | 14 | 0 | 16 | 0 | 1514 | [symbol] | 0 |
| HT [symbol] 10 | | | | | | | | 0 | [symbol] | 0 | 242 | 0 | 443 | [symbol] | 1304 | 0 |
| [symbol] | | | | | | | | 0 | 362 | [symbol] | 91 | [symbol] | 0 | [symbol] | [symbol] | 0 |
| [symbol] | | | | | | | | 0 | 182 | [symbol] | 187 | [symbol] | [symbol] | 0 | 9021 | 0 |
| [symbol] | | | | | | | | 52 | 0 | 0 | 492 | [symbol] | 1535 | 0 | [symbol] | 0 |
| [symbol] | | | | | | | | 1004 | 70 | 0 | 572 | [symbol] | [symbol] | 211 | [symbol] | [symbol] |
| [symbol] | | | | | | | | 1104 | 0 | 0 | 779 | 1305 | [symbol] | [symbol] | 1231 | [symbol] |
| HCT-116-3 | | | | | | | [symbol] | 0 | 0 | 0 | [symbol] | [symbol] | 0 | [symbol] | [symbol] | 0 |
| HCT-116-4 | | | | | | | [symbol] | 0 | 0 | 0 | 0 | [symbol] | 0 | [symbol] | [symbol] | 0 |
| HCT-116-5 | | | | | | | [symbol] | 0 | 0 | 0 | 277 | 14 | 0 | [symbol] | 414 | 0 |
| HCT-116-6 | | | | | | | [symbol] | 0 | 0 | 0 | [symbol] | 0 | 0 | [symbol] | [symbol] | 0 |
| [symbol] -6 | | | | | | | [symbol] | 0 | 0 | 0 | 4346 | 0 | 0 | [symbol] | [symbol] | 0 |
| HT29-3 | | | | | | | [symbol] | 0 | 0 | 0 | 3771 | 100 | 0 | [symbol] | [symbol] | 0 |
| [symbol] -4 | | | | | | | [symbol] | 0 | 0 | 0 | 0 | 0 | 0 | 175 | [symbol] | 0 |
| HT29-4 | | | | | | | [symbol] | 0 | 0 | 0 | 0 | 0 | 0 | [symbol] | [symbol] | 0 |
| HT29-5 | | | | | | | [symbol] | 0 | 0 | 0 | [symbol] | [symbol] | 0 | 234 | 211 | 0 |
| HT29-6 | | | | | | | [symbol] | 0 | 0 | 0 | 0 | [symbol] | 0 | 0 | 910 | 0 |
| OVCAR4-3 | | | | | | | [symbol] | 0 | 0 | 0 | [symbol] | 42 | 0 | 430 | [symbol] | 0 |
| OVCAR4-4 | | | | | | | [symbol] | 0 | 0 | 0 | [symbol] | [symbol] | 0 | 0 | [symbol] | 0 |
| OVCAR4-5 | | | | | | | [symbol] | 0 | 0 | 0 | [symbol] | 734 | 0 | 971 | [symbol] | 0 |
| OVCAR4-6 | | | | | | | [symbol] | 0 | 0 | 0 | [symbol] | [symbol] | 0 | [symbol] | 753 | 0 |
| [symbol] -3 | | | | | | | [symbol] | 0 | 0 | 0 | 0 | 131 | 0 | 315 | [symbol] | 0 |
| [symbol] -4 | | | | | | | [symbol] | 0 | 0 | 0 | 0 | [symbol] | 0 | [symbol] | 524 | 0 |
| [symbol] -5 | | | | | | | [symbol] | 0 | 0 | 0 | [symbol] | 75 | 0 | [symbol] | [symbol] | 0 |
| [symbol] -6 | | | | | | | [symbol] | 0 | 0 | 0 | 617 | 0 | 0 | 0 | 1047 | 0 |
| OVCAR5-3 | | | | | | | [symbol] | 0 | 0 | 0 | 2413 | 62 | 0 | [symbol] | [symbol] | 0 |
| OVCAR5-4 | | | | | | | [symbol] | 0 | 0 | 0 | [symbol] | [symbol] | 0 | 72 | [symbol] | 0 |
| OVCAR4-5 | | | | | | | [symbol] | 0 | 0 | 0 | 444 | 0 | 0 | 0 | 307 | 0 |
| [symbol] -6 | | | | | | | [symbol] | 0 | 0 | 0 | [symbol] | 175 | 0 | 0 | 723 | 0 |
| [symbol] | | | | | | | [symbol] | 0 | 0 | 0 | 2112 | 25 | 0 | 543 | 1131 | 0 |
| HeLa-6 | | | | | | | [symbol] | 0 | 0 | 0 | 0 | [symbol] | 0 | 0 | [symbol] | 0 |
| [symbol] | | | | | | | [symbol] | 0 | 0 | 0 | 358 | 1111 | 0 | [symbol] | 771 | 0 |
| SW480-3 | | | | | | | [symbol] | 0 | 0 | 0 | 226 | 200 | 0 | [symbol] | 641 | 0 |
| SW480-4 | | | | | | | [symbol] | 0 | 0 | 0 | 3044 | 90 | 0 | 0 | [symbol] | 0 |
| SW480-5 | | | | | | | [symbol] | 0 | 0 | 0 | 0 | 577 | 0 | 370 | 1148 | 0 |
| SW480-6 | | | | | | | [symbol] | 0 | 0 | 0 | 240 | 490 | 0 | 1271 | 1247 | 0 |
| C33A-3 | | | | | | | [symbol] | 0 | 0 | 0 | 0 | [symbol] | 0 | 195 | 1104 | 0 |
| C33A-4 | | | | | | | [symbol] | 0 | 0 | 0 | 0 | 0 | 0 | 0 | 521 | 0 |
| C33A-5 | | | | | | | [symbol] | 0 | 0 | 0 | 211 | 0 | 0 | [symbol] | 1143 | 0 |
| C334-6 | | | | | | | [symbol] | 0 | 0 | 0 | [symbol] | 313 | 0 | [symbol] | [symbol] | 0 |
| He [symbol] -6 | | | | | | | [symbol] | 0 | 0 | 0 | 0 | [symbol] | 0 | 0 | [symbol] | 0 |
| [symbol] -3 | | | | | | | [symbol] | 0 | 0 | 0 | [symbol] | [symbol] | 0 | 201 | 724 | 0 |
| [symbol] -4 | | | | | | | [symbol] | 0 | 0 | 0 | 2742 | 15 | 0 | [symbol] | [symbol] | 0 |
| [symbol] -5 | | | | | | | [symbol] | 0 | 0 | 0 | 1376 | 218 | 0 | [symbol] | 715 | 0 |
| [symbol] -6 | | | | | | | [symbol] | 0 | 0 | 0 | [symbol] | 0 | 0 | 1097 | [symbol] | 0 |
| [symbol] -6 | | | | | | | [symbol] | 0 | 0 | 0 | [symbol] | 0 | 0 | 0 | 734 | 0 |
| [symbol] -3 | | | | | | | [symbol] | 0 | 0 | 0 | 4744 | 204 | 0 | 715 | [symbol] | 0 |
| [symbol] -4 | | | | | | | [symbol] | 0 | 0 | 0 | 1334 | [symbol] | 0 | [symbol] | [symbol] | 0 |
| SF-286-3 | | | | | | | [symbol] | 0 | 0 | 0 | 740 | 1244 | 0 | 0 | [symbol] | 0 |
| SF-286-4 | | | | | | | [symbol] | 0 | 0 | 0 | 5402 | [symbol] | 0 | 751 | 721 | 0 |
| SF-286-6 | | | | | | | [symbol] | 0 | 0 | 0 | [symbol] | [symbol] | 0 | 0 | [symbol] | 0 |
| SF-286-5 | | | | | | | [symbol] | 0 | 0 | 0 | [symbol] | 0 | 0 | 10 | 790 | 0 |
| [symbol] -13 | | | | | | | | 0 | 0 | 0 | [symbol] | 47 | 0 | [symbol] | [symbol] | 0 |
| [symbol] -20 | | | | | | | | 0 | 0 | 0 | [symbol] | 0 | 0 | 0 | [symbol] | 0 |
| [symbol] -21 | | | | | | | | 0 | 0 | 0 | [symbol] | 70 | 0 | [symbol] | 5311 | 0 |
| [symbol] -22 | | | | | | | | 0 | 0 | 0 | [symbol] | 234 | 0 | 227 | [symbol] | 0 |
| [symbol] -5 | | | | | | | [symbol] | 0 | 0 | 0 | 0 | 95 | 0 | 312 | 741 | 0 |
| [symbol] -10 | | | | | | | | 0 | 0 | 0 | 15 | 3 | 0 | [symbol] | [symbol] | 0 |
| [symbol] -11 | | | | | | | | 0 | 0 | 0 | 0 | [symbol] | 0 | 1 | [symbol] | 0 |
| [symbol] -12 | | | | | | | | 0 | 0 | 0 | 0 | [symbol] | 0 | 2134 | 901 | 0 |
| [symbol] -13 | | | | | | | | 0 | 0 | 0 | 0 | 1044 | 0 | [symbol] | [symbol] | 0 |
| [symbol] -14 | | | | | | | | 0 | 0 | 0 | 0 | 207 | 0 | 921 | [symbol] | 0 |
| [symbol] -15 | | | | | | | | 0 | 0 | 0 | 0 | [symbol] | 0 | [symbol] | [symbol] | 0 |
| [symbol] -16 | | | | | | | | 0 | 0 | 0 | 0 | 2 | 0 | 0 | [symbol] | 0 |
| [symbol] -17 | | | | | | | | 0 | 0 | 0 | 0 | 0 | 0 | 0 | [symbol] | 0 |
| [symbol] -18 | | | | | | | | 0 | 0 | 0 | 567 | 33 | 0 | 1234 | [symbol] | 0 |
| [symbol] -19 | | | | | | | | 0 | 0 | 0 | 4 | 55 | 0 | [symbol] | [symbol] | 0 |

| T [symbol] | T [symbol] | [symbol] | T [symbol] | T [symbol] | [symbol] | B [symbol] | p53 | sEQ 110 [symbol] |
|---|---|---|---|---|---|---|---|---|
| [symbol] | | 1 | | | | | | [symbol] |
| [symbol] | | 2 | | | | | | 834 |
| [symbol] | | 3 | | | | | | 822 |
| [symbol] | | 4 | | | | | | 0 |
| [symbol] | | 5 | | | | | | [symbol] |
| [symbol] | | 6 | | | | | | [symbol] |
| [symbol] | | 7 | | | | | | [symbol] |
| [symbol] | | 8 | | | | | | [symbol] |
| [symbol] | | 9 | | | | | | 2327 |
| [symbol] | | 10 | | | | | | 2719 |
| [symbol] | | 11 | | | | | | [symbol] |
| [symbol] | | 12 | | | | | | [symbol] |
| [symbol] | | 13 | | | | | | [symbol] |
| [symbol] | | 14 | | | | | | [symbol] |
| [symbol] | | 15 | | | | | | 410 |
| [symbol] | | 16 | | | | | | 0 |
| [symbol] | | 17 | | | | | | [symbol] |
| [symbol] | | 18 | | | | | | [symbol] |
| [symbol] | | 19 | | | | | | 282 |
| [symbol] | | 20 | | | | | | 290 |
| [symbol] | | 21 | | | | | | 51 |
| [symbol] | | 22 | | | | | | [symbol] |
| [symbol] | | 23 | | | | | | [symbol] |
| [symbol] | | 24 | | | | | | [symbol] |
| [symbol] | | 25 | | | | | | [symbol] |
| [symbol] | | 27 | | | | | | 141 |
| [symbol] | | 28 | | | | 28 | | 332 |
| [symbol] | | 29 | | | | | | 0 |
| RPTEC | | 30 | | | | 30 | | [symbol] |
| [symbol] | | 31 | | | | | | [symbol] |
| MMEC | | 32 | | | | | | [symbol] |
| [symbol] | | 33 | | | | | | [symbol] |
| [symbol] | | 34 | | | | | | [symbol] |
| [symbol] | | 35 | | | | | | 0 |
| [symbol] | | 36 | | | | | | 17 [symbol] |
| [symbol] | | 37 | | | | | | 71 |
| [symbol] | | 38 | | | | | | 1004 |
| [symbol] | | 39 | | | | | | 102 |
| [symbol] | | 40 | | | | | | 14 [symbol] |
| [symbol] | | 41 | | | | | | 120 |
| [symbol] | | 42 | | | | | | [symbol] 37 |
| [symbol] | | 43 | | | | | | 140 |
| [symbol] | | 44 | | | | | | 0 |
| [symbol] | | | | | 305 | | | 0 |
| [symbol] | | | | | [symbol] | | | 0 |
| [symbol] | | | | | 341 | | | 0 |
| [symbol] | | | | | 358 | 358 | | [symbol] |
| [symbol] | | | | | 364 | 354 | | [symbol] |
| [symbol] | | | | | 344 | | | [symbol] |
| [symbol] | | | | | 342 | | | 0 |
| RPTEC | | | | | 334 | 334 | | 0 |
| [symbol] | | | | | 322 | | | [symbol] |
| [symbol] | | | | | 330 | | | 141 |
| [symbol] | | | | | 329 | | | 70 |
| [symbol] | | | | | 327 | | | [symbol] |
| [symbol] | | | | | [symbol] | | | [symbol] |
| [symbol] | | | | | 321 | | | 0 |
| [symbol] | | | | | 320 | | | 0 |
| [symbol] | | | | | [symbol] | | | 140 |
| [symbol] | | | | | [symbol] | | | 203 |
| [symbol] | | | | | 314 | | | 173 |
| [symbol] | | | | | 311 | | | 0 |
| [symbol] | | | | | [symbol] | | | [symbol] |
| [symbol] | | | | | 307 | | | [symbol] |
| [symbol] | | | | | 305 | | | 0 |
| [symbol] | | | | | 303 | | | 0 |
| [symbol] | | | | | 302 | | | 307 |
| [symbol] | | | | | 290 | | | 18 |
| [symbol] | | | | | 291 | | | [symbol] |
| [symbol] | | | | | [symbol] | | | 21 |
| [symbol] | | | | | [symbol] | | | 111 |
| [symbol] | | | | | [symbol] | | | 0 |
| [symbol] | | | | | [symbol] | | | 0 |
| [symbol] | | | | | 279 | | | [symbol] |
| [symbol] | | | | | 277 | | | 134 |
| HPAEC | | | | | 275 | 275 | | 0 |
| [symbol] | | | | | [symbol] | | | 0 |
| [symbol] | | | | | [symbol] | | | [symbol] |
| [symbol] | | | | | 230 | 230 | | 331 |
| [symbol] | | | | | 233 | 233 | | 222 |
| MT373 [symbol] | | | | | 234 | | | 0 |
| [symbol] | | | | | 233 | 233 | | 356 |
| [symbol] | | | | | 221 | [symbol] | | [symbol] |
| [symbol] | | | | | 229 | 229 | | [symbol] |
| [symbol] | | | | | 227 | 227 | | 442 |
| [symbol] | | | | | 222 | | | [symbol] |
| [symbol] | | | | | 218 | | | 0 |
| [symbol] | | | | | 214 | | | [symbol] |
| [symbol] | | | | | 213 | | | 0 |
| [symbol] | | | | | 212 | | | [symbol] |
| HCAEC | | | | | 211 | 211 | | 0 |
| [symbol] | | | | | 210 | | | 0 |
| MMEC | | | | | 209 | | | 0 |
| [symbol] | | | | | [symbol] | | | [symbol] |
| [symbol] | | | | | [symbol] | | | 0 |
| [symbol] | | | | | 201 | | | 0 |
| [symbol] | | | | | 190 | | | 0 |
| [symbol] | | | | | [symbol] | | | 0 |
| [symbol] | | | | | [symbol] | | | [symbol] |
| [symbol] | | | | | [symbol] | | | 0 |
| [symbol] | | | | | [symbol] | | | 0 |
| [symbol] | | | | | [symbol] | | | 0 |
| [symbol] | | | | | [symbol] | | | 0 |
| [symbol] | | | | | [symbol] | | | 0 |
| [symbol] | | | | | [symbol] | | | 0 |
| [symbol] | | | | | [symbol] | | | 0 |
| [symbol] | | | | | 51 | | | [symbol] |
| [symbol] | | | | | 49 | | | 0 |
| MELA [symbol] | | | | 79 | | | | 0 |
| MELA [symbol] | | | | 81 | | | | [symbol] |
| MELA [symbol] | | | | 83 | | | | [symbol] |
| MELA [symbol] | | | | [symbol] | | | | [symbol] |
| MELA [symbol] | | | | [symbol] | | | | 87 |
| MELA [symbol] | | | | [symbol] | | | | [symbol] |
| MELA [symbol] | | | | [symbol] | | | | 0 |
| [symbol] | | | | [symbol] | | | | 91 |
| [symbol] | | | | [symbol] | | | | 0 |
| [symbol] | | | | [symbol] | | | | 120 |
| [symbol] | | | | 140 | | | | [symbol] |
| [symbol] | | | | 150 | | | | [symbol] |
| [symbol] | | | | 152 | | | | [symbol] |
| [symbol] | | | | 154 | | | | 0 |
| SF [symbol] | | | | [symbol] | | | | 137 |
| SF [symbol] | | | | 15 [symbol] | | | | 0 |
| CC [symbol] | | | | 1 [symbol] | | | | 185 |
| OU-145 | | | | 1 [symbol] 2 | | | | 0 |
| MCT [symbol] | | | | 1 [symbol] 4 | | | | 0 |
| [symbol] | | | | [symbol] | | | | 4 |
| 766-4 | | | | [symbol] | | | | 0 |
| T-47D | | | | [symbol] | | | | 0 |
| [symbol] | | | | 177 | | | | [symbol] |
| CPI 1441 [symbol] | | | | 181 | | | | 0 |
| 79T [symbol] | | | | 183 | | | | 100 |
| [symbol] A* | | | | 184 | | | | 47 |
| [symbol] poly A* | | | | 114 | | | | 0 |
| AC [symbol] | | | | 114 | | | | 62 |
| [symbol] CC-42 | | | | 200 | | | | 0 |
| MCF-7 [symbol] | | | | 222 | | | | [symbol] |
| UTC ( [symbol] ) [symbol] | | | | 234 | | | | 0 |
| [symbol] poly | | | | [symbol] | | | | 34 |
| 444 [symbol] | | | | 200 | | | | 230 |
| CCI, T37 RMA 37 [symbol] | | | | 218 | | | | 0 |
| [symbol] 200 10% FBS | | | | 218 | | | | 4 |
| [symbol] | | | | 239 | | | | 242 |
| [symbol] -1 | | | | 221 | | | | [symbol] |
| [symbol] -2 | | | | 223 | | | | 79 |
| [symbol] -4 | | | | 224 | | | | 0 |
| [symbol] | | | | 241 | | | | 0 |
| [symbol] T-4 | | | | 242 | | | | 0 |
| [symbol] | | | | 243 | | | | 0 |
| [symbol] | | | | 244 | | | | 0 |
| [symbol] | | | | 245 | | | | 0 |
| [symbol] | | | | 246 | | | | [symbol] |
| [symbol] TCC | | | | 247 | | | | 241 |
| SR | | | | 248 | | | | 0 |
| OVCAR-3 | | | | 249 | | | | 85 |
| HCT-15 | | | | 250 | | | | 0 |
| OVCAR-4 | | | | 251 | | | | 0 |
| UO-31 | | | | 252 | | | | 0 |
| OVCAR-4 | | | | 253 | | | | 248 |
| [symbol] 12C | | | | 254 | | | | 0 |
| OVCAR-5 | | | | 255 | | | | 0 |
| LCD [symbol] | | | | 256 | | | | 0 |
| [symbol] | | | | 257 | | | | 0 |
| [symbol] -2 | | | | 258 | | | | 153 |
| [symbol] -3 | | | | 259 | | | | 0 |
| [symbol] -4 | | | | 260 | | | | 0 |
| SF- [symbol] | | | | 261 | | | | 0 |
| [symbol] | | | | 262 | | | | 0 |
| [symbol] | | | | 263 | | | | 22 |
| UACC-257 | | | | 264 | | | | 33 |
| [symbol] 14 | | | | 265 | | | | 0 |
| MCF7 | | | | 267 | | | | 43 |
| MCA-A [symbol] -435 | | | | [symbol] | | | | 100 |
| [symbol] T270 | | | | 270 | | | | 0 |
| MCA-4 | | | | 271 | | | | 0 |
| Y79 poly A* | | | | 273 | | | | 0 |
| IO [symbol] poly A* | | | | [symbol] | | | | 34 |
| HT836 [symbol] TPA [symbol] | | | | 300 | | | | 147 |
| [symbol] | | | | 313 | | | | 83 |
| [symbol] | | | | 322 | | | | 0 |
| 44T347 | | | | 333 | | | | 0 |
| [symbol] | | | | 334 | | | | 127 |
| [symbol] | | | | 234 | | | | 0 |
| [symbol] | | | | 337 | | | | 0 |
| [symbol] | | | | [symbol] | | | | 61 |
| U29 | | | | 330 | | | | 8 |
| PT [symbol] A* | | | | 300 | | | | 0 |
| PC-3 | | | | 341 | | | | 0 |
| MCC-7000 | | | | 343 | | | | 0 |
| SW-420 | | | | 345 | | | | 0 |
| [symbol] TT82 | | | | 346 | | | | 148 |
| CCLO 293 | | | | 347 | | | | 0 |
| HT294 | | | | 348 | | | | 0 |
| [symbol] -12 | | | | 34349 | | | | 0 |
| XT151 | | | | 350 | | | | 20 |
| AA95 | | | | 351 | | | | 0 |
| HT [symbol] | | | | 352 | | | | 140 |
| [symbol] | | | | 353 | | | | 0 |
| TK-19 | | | | 355 | | | | 363 |
| [symbol] | | | | 357 | | | | 241 |
| [symbol] 578T | | | | 350 | | | | 0 |
| HT13 | | | 50 | | | | | 312 |
| HT [symbol] | | | 52 | | | | | 0 |
| HT130 | | | 54 | | | | | 0 |
| HT166 | | | 56 | | | | | 0 |
| HT183 | | | 58 | | | | | 165 |
| HT170 | | | 60 | | | | | 61 |
| HT172 | | | 62 | | | | | 133 |
| HT130 | | | 63 | | | | | 0 |
| HT176 | | | 64 | | | | | 32 |
| HT154 | | | 65 | | | | | 19 |
| HT183 | | | 66 | | | | | 40 |
| HT100 | | | 67 | | | | | 140 |
| HT100 | | | 68 | | | | | 0 |
| HT143 | | | 69 | | | | | 0 |
| HT180 | | | 70 | | | | | [symbol] |
| HT [symbol] | | | 71 | | | | | 130 |
| HT227 | | | 72 | | | | | 111 |
| HT322 | | | 73 | | | | | 0 |
| HT314 | | | 74 | | | | | 0 |
| HT317 | | | 76 | | | | | 0 |
| [symbol] | | | 77 | | | | | 0 |
| HT323 | | | 78 | | | | | 21 |
| HT327 | | | 80 | | | | | 137 |
| HT335 | | | 82 | | | | | 0 |
| HT144 | | | 85 | | | | | [symbol] |
| HT346 | | | 87 | | | | | 29 |
| HT311 | | | 170 | | | | | 18 |
| HT [symbol] | | | 185 | | | | | 0 |
| HT140 | | | 187 | | | | | 0 |
| HT201 | | | 190 | | | | | 236 |
| HT372 | | | 191 | | | | | 75 |
| TCCP | | | 207 | | | | | 40 |
| HT [symbol] | | | 214 | | | | | 44 |
| HT307 | | | 217 | | | | | 82 |
| HT [symbol] | | | 224 | | | | | 258 |
| HT370 | | | 226 | | | | | 0 |
| HT371 | | | 228 | | | | | 114 |
| HT377 | | | 230 | | | | | 125 |
| HT382 | | | [symbol] | | | | | 112 |
| [symbol] | | | [symbol] | | | | | 445 |
| HT334 | | | 290 | | | | | 231 |
| HT328 | | | 301 | | | | | 0 |
| HT302 | | | 315 | | | | | [symbol] 3 |
| HT304 | | | 317 | | | | | 0 |
| HT312 | | | 318 | | | | | 0 |
| HT162 | | | 325 | | | | | 0 |
| HT304 | | | 354 | | | | | 45 |
| HT187 | | | 300 | | | | | 13 |
| T-47D | 103 | | | | | | | 83 |
| [symbol] | 101 | | | | | | | 0 |
| [symbol] -423 | 154 | | | | | | | 0 |
| [symbol] | 157 | | | | | | | [symbol] |
| [symbol] | [symbol] | | | | | | | [symbol] |
| [symbol] | [symbol] | | | | | | | [symbol] |
| [symbol] | 151 | | | | | | | [symbol] |
| [symbol] | [symbol] | | | | | | | 7 |
| [symbol] | [symbol] | | | | | | | [symbol] |
| [symbol] | [symbol] | | | | | | | 0 |
| [symbol] | 144 | | | | | | | [symbol] |
| [symbol] | 143 | | | | | | | 0 |
| [symbol] | 142 | | | | | | | [symbol] |
| [symbol] | 141 | | | | | | | 4 |
| [symbol] | 140 | | | | | | | [symbol] |
| [symbol] | 139 | | | | | | | [symbol] |
| [symbol] | 138 | | | | | | | [symbol] |
| [symbol] | 137 | | | | | | | [symbol] |
| [symbol] | [symbol] | | | | | | | 0 |
| [symbol] | [symbol] | | | | | | | 0 |
| [symbol] | 134 | | | | | | | 0 |
| [symbol] | 133 | | | | | | | 0 |
| [symbol] | 132 | | | | | | | [symbol] |
| [symbol] | 131 | | | | | | | [symbol] |
| [symbol] | 130 | | | | | | | [symbol] |
| PC-3 | 129 | | | | | | | 0 |
| [symbol] | 128 | | | | | | | [symbol] |
| [symbol] | 127 | | | | | | | [symbol] |
| [symbol] | 126 | | | | | | | [symbol] |
| [symbol] | 125 | | | | | | | 0 |
| [symbol] | 124 | | | | | | | 137 |
| [symbol] | 123 | | | | | | | 0 |
| [symbol] | 122 | | | | | | | 138 |
| [symbol] | 121 | | | | | | | 0 |
| [symbol] | 120 | | | | | | | 118 |
| OVCAR-4 | 119 | | | | | | | 0 |
| [symbol] | 118 | | | | | | | 34 |
| OVCAR-4 | 117 | | | | | | | [symbol] |
| [symbol] | 116 | | | | | | | [symbol] |
| OVCAR-3 | 115 | | | | | | | [symbol] |
| [symbol] | 114 | | | | | | | 47 |
| [symbol] | 113 | | | | | | | 0 |
| [symbol] | 112 | | | | | | | 141 |
| [symbol] | 111 | | | | | | | 110 |
| [symbol] | 110 | | | | | | | [symbol] |
| [symbol] | 109 | | | | | | | 0 |
| [symbol] | 108 | | | | | | | [symbol] |
| [symbol] | 107 | | | | | | | 0 |
| [symbol] | 106 | | | | | | | 20 |
| [symbol] | 105 | | | | | | | 430 |
| [symbol] | 104 | | | | | | | 78 |
| [symbol] | 103 | | | | | | | 92 |
| [symbol] | 102 | | | | | | | 0 |
| [symbol] | 101 | | | | | | | 777 |
| [symbol] | 100 | | | | | | | 0 |
| [symbol] | 99 | | | | | | | 54 |
| OVCAR-4 | 98 | | | | | | | 0 |
| [symbol] | 97 | | | | | | | 43 |
| [symbol] | 48 | | | | | | | 148 |
| [symbol] | 47 | | | | | | | 210 |
| [symbol] | [symbol] | | | | | | | [symbol] |
| [symbol] | [symbol] | | | | | | | 0 |
| [symbol] -1 | | | | | | | [symbol] | 5 |
| [symbol] -3 | | | | | | | [symbol] | 379 |
| [symbol] -4 | | | | | | | [symbol] | [symbol] |
| [symbol] -5 | | | | | | | [symbol] | [symbol] |
| [symbol] -7 | | | | | | | [symbol] | 73 |
| EXVX-1 | | | | | | | [symbol] | 94 |
| EXVX-4 | | | | | | | [symbol] | [symbol] |
| EXVX-5 | | | | | | | [symbol] | 0 |
| EXVX-6 | | | | | | | [symbol] | [symbol] |
| EXVX-7 | | | | | | | [symbol] | 677 |
| MCF-7-1 | | | | | | | [symbol] | 0 |
| MCF-7-3 | | | | | | | [symbol] | [symbol] |
| MCF-7-4 | | | | | | | [symbol] | 318 |
| MCF-7-6 | | | | | | | [symbol] | 0 |
| MCF-7-7 | | | | | | | [symbol] | 0 |
| [symbol] -1 | | | | | | | [symbol] | [symbol] |
| [symbol] -3 | | | | | | | [symbol] | 198 |
| [symbol] -4 | | | | | | | [symbol] | 73 |
| [symbol] -6 | | | | | | | [symbol] | 19 |
| [symbol] -7 | | | | | | | [symbol] | [symbol] |
| WI 34-1 | | | | | | | [symbol] | 0 |
| WI 34-3 | | | | | | | [symbol] | 283 |
| WI 34-4 | | | | | | | [symbol] | [symbol] |
| WI 34-6 | | | | | | | [symbol] | [symbol] |
| WI 34-7 | | | | | | | [symbol] | 0 |
| [symbol] -1 | | | | | | | [symbol] | 0 |
| [symbol] -3 | | | | | | | [symbol] | [symbol] |
| [symbol] -4 | | | | | | | [symbol] | [symbol] |
| [symbol] -5 | | | | | | | [symbol] | [symbol] |
| [symbol] -7 | | | | | | | [symbol] | 105 |
| [symbol] -1 | | | | | | | [symbol] | 0 |
| [symbol] -3 | | | | | | | [symbol] | 0 |
| [symbol] -4 | | | | | | | [symbol] | 0 |
| [symbol] -5 | | | | | | | [symbol] | 0 |
| [symbol] -7 | | | | | | | [symbol] | 1 |
| [symbol] -2 | | | | | | | [symbol] | [symbol] |
| [symbol] -2 | | | | | | | [symbol] | 0 |
| [symbol] -1 | | | | | | | [symbol] | 0 |
| [symbol] -2 | | | | | | | [symbol] | [symbol] |
| [symbol] -2 | | | | | | | [symbol] | [symbol] |
| [symbol] -1 | | | | | | | [symbol] | 0 |
| [symbol] -2 | | | | | | | [symbol] | 3 |
| [symbol] -1 | | | | | | | [symbol] | 204 |
| [symbol] -2 | | | | | | | [symbol] | 831 |
| OVCAR-4-1 | | | | | | | [symbol] | 0 |
| OVCAR-4-2 | | | | | | | [symbol] | 0 |
| OVCAR-4-1 | | | | | | | [symbol] | 123 |
| OVCAR-4-2 | | | | | | | [symbol] | 234 |
| [symbol] -2 | | | | | | | [symbol] | [symbol] |
| [symbol] -2 | | | | | | | [symbol] | 271 |
| [symbol] -2 | | | | | | | [symbol] | 173 |
| [symbol] -1 | | | | | | | [symbol] | 0 |
| [symbol] -2 | | | | | | | [symbol] | [symbol] |
| [symbol] -2 | | | | | | | [symbol] | [symbol] |
| [symbol] -1 | | | | | | | [symbol] | 0 |
| [symbol] -2 | | | | | | | [symbol] | 0 |
| [symbol] -1 | | | | | | | [symbol] | 271 |
| [symbol] -2 | | | | | | | [symbol] | [symbol] |
| [symbol] -1 | | | | | | | [symbol] | 0 |
| [symbol] -2 | | | | | | | [symbol] | 24 |
| [symbol] -2 | | | | | | | | [symbol] |
| [symbol] -1 | | | | | | | | 0 |
| [symbol] -2 | | | | | | | | 0 |
| [symbol] | | | | | | | | 0 |
| [symbol] | | | | | | | | [symbol] |
| [symbol] | | | | | | | | [symbol] |
| [symbol] | | | | | | | | 0 |
| [symbol] | | | | | | | | 0 |
| [symbol] | | | | | | | | 0 |
| [symbol] | | | | | | | | [symbol] |
| [symbol] -4 | | | | | | | | 2301 |
| [symbol] -9 | | | | | | | | [symbol] |
| [symbol] -11 | | | | | | | | 2522 |
| [symbol] -12 | | | | | | | | [symbol] |
| [symbol] -10 | | | | | | | | 300 |
| [symbol] -1 | | | | | | | | 474 |
| [symbol] -2 | | | | | | | | 0 |
| [symbol] -3 | | | | | | | | 222 |
| [symbol] -4 | | | | | | | | 0 |
| [symbol] -5 | | | | | | | | 0 |
| [symbol] -6 | | | | | | | | 0 |
| [symbol] -8 | | | | | | | wt | 0 |
| EXVX-8 [symbol] | | | | | | | [symbol] | 50 |
| HCT-1 16-1 | | | | | | | wt | 0 |
| HCT-1 14-6 | | | | | | | wt | 0 |
| WT29-1 | | | | | | | [symbol] | 310 |
| WT29-7 | | | | | | | [symbol] | [symbol] |
| WT29-8 | | | | | | | [symbol] | 0 |
| SF434-7 | | | | | | | wt | 155 |
| SF434-8 | | | | | | | wt | 228 |
| SF200-7 | | | | | | | [symbol] | 218 |
| SF300-8 | | | | | | | [symbol] | [symbol] |
| OVCAR-4-7 | | | | | | | wt | 493 |
| OVCAR-4-8 | | | | | | | wt | 0 |
| OVCAR-5-7 | | | | | | | [symbol] | 531 |
| OVCAR-5-8 | | | | | | | [symbol] | [symbol] |
| [symbol] -7- [symbol] | | | | | | | wt | 33 |
| [symbol] -8 | | | | | | | [symbol] | 837 |
| [symbol] -8 | | | | | | | HPV E8 | 0 |
| SW420-7 | | | | | | | [symbol] | 0 |
| SW430-8 | | | | | | | [symbol] | 0 |
| Wt200-5 | | | | | | | [symbol] | 0 |
| C33A-7 | | | | | | | [symbol] | 0 |
| C33A-8 | | | | | | | [symbol] | 0 |
| U2OA-7 | | | | | | | [symbol] | 720 |
| U2OA-8 | | | | | | | [symbol] | 37 |
| [symbol] -7 | | | | | | | wt | 0 |
| [symbol] -8 | | | | | | | wt | 0 |
| WI30-8 | | | | | | | wt | 0 |
| [symbol] | | | | | | | | 0 |
| [symbol] , 1572, [symbol] | | | | | | | | 71 |
| [symbol] 4 | | | | | | 64 | | 221 |
| WT268 | | | | | | | | 80 |
| WT376 | | | | | | | | 183 |
| WT395 | | | | | | | | 88 |
| WT320 | | | | | | | | 0 |
| [symbol] -3 | | | | | | 173 | | 487 |
| [symbol] -6 | | | | | | 176 | | 117 |
| [symbol] -9 | | | | | | 177 | | 481 |
| [symbol] | | | | | | | | 0 |
| [symbol] | | | | | | 233 | | 0 |
| [symbol] | | | | | | | | 0 |
| [symbol] Mar. 31, 2002 [symbol] | | | | | | | | 0 |
| [symbol] | | | | | | | | 0 |
| [symbol] | | | | | | | | 22 |
| [symbol] | | | | | | | | 0 |
| [symbol] | | | | | | | | 0 |
| HCT-114-3 | | | | | | | wt | [symbol] |
| HCT-114-4 | | | | | | | wt | 0 |
| HCT-114-5 | | | | | | | wt | 0 |
| HCT-114-6 | | | | | | | wt | 0 |
| [symbol] -6 | | | | | | | wt | 0 |
| WT28-3 | | | | | | | [symbol] | 874 |
| EXVX-6 | | | | | | | [symbol] | 129 |
| WT29-4 | | | | | | | [symbol] | 640 |
| WT29-5 | | | | | | | [symbol] | 0 |
| WT29-6 | | | | | | | [symbol] | 177 |
| OVCAR-4-3 | | | | | | | wt | 220 |
| OVCAR-4-4 | | | | | | | wt | 0 |
| OVCAR-4-5 | | | | | | | wt | [symbol] |
| OVCAR-4-6 | | | | | | | wt | 0 |
| SF [symbol] -3 | | | | | | | wt | 728 |
| SF [symbol] -4 | | | | | | | wt | 83 |
| SF [symbol] -5 | | | | | | | wt | 0 |
| SF [symbol] -6 | | | | | | | wt | 0 |
| OVCAR-5-3 | | | | | | | [symbol] | 2718 |
| OVCAR-5-4 | | | | | | | [symbol] | 296 |
| OVCAR-5-5 | | | | | | | [symbol] | 0 |
| OVCAR-5-6 | | | | | | | [symbol] | 0 |
| MCF-7-6 | | | | | | | wt | 270 |
| [symbol] | | | | | | | HPV E5 | 371 |
| [symbol] | | | | | | | [symbol] | 0 |
| SW400-3 | | | | | | | [symbol] | 61 |
| SW400-4 | | | | | | | [symbol] | 0 |
| SW400-5 | | | | | | | [symbol] | 293 |
| SW400-6 | | | | | | | [symbol] | 225 |
| C33A-3 | | | | | | | [symbol] | 173 |
| C33A-4 | | | | | | | [symbol] | [symbol] |
| C33A-5 | | | | | | | [symbol] | 0 |
| C33A-6 | | | | | | | [symbol] | 56 |
| [symbol] -6 | | | | | | | wt | 0 [symbol] |
| U2O3-3 | | | | | | | [symbol] | 630 |
| U2O3-4 | | | | | | | [symbol] | [symbol] |
| U2O3-5 | | | | | | | [symbol] | 544 |
| U2O3-6 | | | | | | | [symbol] | 0 |
| Wt 30- [symbol] | | | | | | | wt | 0 |
| [symbol] -3 | | | | | | | wt | [symbol] |
| [symbol] -4 | | | | | | | wt | [symbol] |
| SF-200-3 | | | | | | | [symbol] | 277 |
| SF-200-4 | | | | | | | [symbol] | 0 |
| SF-200-5 | | | | | | | [symbol] | 123 |
| SF-200-6 | | | | | | | [symbol] | 33 |
| [symbol] -13 | | | | | | | | 21 |
| [symbol] -20 | | | | | | | | [symbol] |
| [symbol] -21 | | | | | | | | 0 |
| [symbol] -22 | | | | | | | | 0 |
| OVCAR-5-6 | | | | | | | [symbol] | [symbol] |
| [symbol] -10 | | | | | | | | [symbol] |
| [symbol] -11 | | | | | | | | [symbol] |
| [symbol] -12 | | | | | | | | [symbol] |
| [symbol] -13 | | | | | | | | 320 |
| [symbol] -14 | | | | | | | | 150 |
| [symbol] -15 | | | | | | | | 332 |
| [symbol] -16 | | | | | | | | 0 |
| [symbol] -17 | | | | | | | | 123 |
| [symbol] -18 | | | | | | | | 0 |
| [symbol] -19 | | | | | | | | 172 |

TABLE 4

| Gene Name | SP | ID# na | ID# aa | Family | Group | Length_AA | Extra-Catalytic Domains (Amino acid positions) |
|---|---|---|---|---|---|---|---|
| X69117_h beta_adrene | H | 1 | 122 | AGC | GRK | 688 | Regulator of G protein signating domain 54-175; |
| | | | | | | | PH domain 559-652 |
| AA144574_m | M | 2 | 123 | AGC | GRK | 378 | PH domain 249-337 |
| AA210825_h | H | 9 | 130 | AGC | PKC | 978 | Phorbol esters/diacylglycerol binding domain |
| | | | | | | | (C1 domain) 238-287; PH domain 497-577 |
| AA316804_h | H | 11 | 132 | AGC | PKC | 890 | Phorbol esters/diacylglycerol binding domain |
| | | | | | | | (C1 domain) 155-204 and 272-321; PH domain 417-532 |
| AA887763_h | H | 21 | 142 | AGC | SGK | 446 | PX domain 13-120 |
| AA021445_h 3 | H | 32 | 152 | CAMK | EMK | 1311 | Vitamin K-dependent carboxylation/gamma-carboxyglutamic |
| | | | | | | | (GLA) domain 1072-1113 |
| R31237_1_h, AAC3348 | H | 34 | 154 | CAMK | EMK | 729 | UBA domain 327-365 |
| 408786.5_h | H | 36 | 156 | CAMK | EMK | 1330 | PAS domain 133-186, 247-280, 354-386 |
| Z36720_h | H | 41 | 161 | CAMK | MLCK | 874 | WD domain, G-beta repeat 674-711 |
| SGK088_h | H | 42 | 162 | CAMK | Trio | 2287 | Immunoglobulin domain 1-62, 97-153, 221-277, |
| | | | | | | | 518-578, 1617-1678; Fibronectin type III domain 301-390, |
| | | | | | | | 1697-1779 |
| R19772_h | H | 44 | 164 | CAMK | Trio | 1287 | RhoGEF domain 235-405; Fibronectin type III domain |
| | | | | | | | 870-955; Immunoglobulin domain 786-851; PH |
| | | | | | | | domain 419-528 |
| 17000139801197_h, IRA | H | 76 | 195 | Other | IRAK | 596 | Death domain 26-106 |
| AA088547_h | H | 78 | 197 | Other | IRE | 922 | PQQ enzyme repeat 39-76 |
| AA232253_h | H | 82 | 201 | Other | MLK | 800 | SAM domain (Sterlie alpha motif) 337-408 |
| AA599286_h | H | 89 | 208 | Other | SLOB | 649 | PX domain 16-122 |
| AA838348_h | H | 113 | 232 | STE | NEK | 836 | Regulator of chromosome condensation (RCC1) 387-427, |
| | | | | | | | 427-480, 483-532, 598-650 |
| PAK6_h | H | 115 | 234 | STE | STE20-02 | 719 | P21-Rho-binding domain 11-69 |

1-25. (canceled)

26. An antibody or antibody fragment having specific binding affinity to a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242.

27. The antibody or antibody fragment of claim 26, wherein said polypeptide comprises:
(a) an amino acid sequence selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242;
(b) an amino acid sequence selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242, except that it lacks one or more, but not all, of the domains selected from the group consisting of a C-terminal domain, a catalytic domain, an N-terminal domain, a spacer region, a praline-rich region, a coiled-coil structure region, and a C-terminal tail.
(c) a domain of an amino acid sequence selected from the group set forth in SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242 wherein said domain is selected from the group consisting of a C-terminal domain, a catalytic domain, an N-terminal domain, a spacer region, a praline-rich region, a coiled-coil structure region, and a C-terminal tail.

28. A hybridoma which produces an antibody having specific binding affinity to a kinase polypeptide selected from the group consisting of SEQ ID NO:122, SEQ ID NO:123, SEQ ID NO:124, SEQ ID NO:125, SEQ ID NO:126, SEQ ID NO:127, SEQ ID NO:128, SEQ ID NO:129, SEQ ID NO:130, SEQ ID NO:131, SEQ ID NO:132, SEQ ID NO:133, SEQ ID NO:134, SEQ ID NO:135, SEQ ID NO:136, SEQ ID NO:137, SEQ ID NO:138, SEQ ID NO:139, SEQ ID NO:140, SEQ ID NO:141, SEQ ID NO:142, SEQ ID NO:143, SEQ ID NO:144, SEQ ID NO:145, SEQ ID NO:146, SEQ ID NO:147, SEQ ID NO:148, SEQ ID NO:149, SEQ ID NO:150, SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160, SEQ ID NO:161, SEQ ID NO:162, SEQ ID NO:163, SEQ ID NO:164, SEQ ID NO:165, SEQ ID NO:166, SEQ ID NO:167, SEQ ID NO:168, SEQ ID NO:169, SEQ ID NO:170, SEQ ID NO:171, SEQ ID NO:172, SEQ ID NO:173, SEQ ID NO:174, SEQ ID NO:175, SEQ ID NO:176, SEQ ID NO:177, SEQ ID NO:178, SEQ ID NO:179, SEQ ID NO:180, SEQ ID NO:181, SEQ ID NO:182, SEQ ID NO:183, SEQ ID NO:184, SEQ ID NO:185, SEQ ID NO:186, SEQ ID NO:187, SEQ ID NO:189, SEQ ID NO:190, SEQ ID NO:191, SEQ ID NO:192, SEQ ID NO:193, SEQ ID NO:194, SEQ ID NO:195, SEQ ID NO:196, SEQ ID NO:197, SEQ ID NO:198, SEQ ID NO:199, SEQ ID NO:200, SEQ ID NO:201, SEQ ID NO:202, SEQ ID NO:203, SEQ ID NO:204, SEQ ID NO:205, SEQ ID NO:206, SEQ ID NO:207, SEQ ID NO:208, SEQ ID NO:209, SEQ ID NO:210, SEQ ID NO:211, SEQ ID NO:212, SEQ ID NO:213, SEQ ID NO:214, SEQ ID NO:215, SEQ ID NO:216, SEQ ID NO:217, SEQ ID NO:218, SEQ ID NO:219, SEQ ID NO:220, SEQ ID NO:221, SEQ ID NO:222, SEQ ID NO:223, SEQ ID NO:224, SEQ ID NO:225, SEQ ID NO:226, SEQ ID NO:227, SEQ ID NO:228, SEQ ID NO:229, SEQ ID NO:230, SEQ ID NO:231, SEQ ID NO:232, SEQ ID NO:233, SEQ ID NO:234, SEQ ID NO:235, SEQ ID NO:236, SEQ ID NO:237, SEQ ID NO:238, SEQ ID NO:239, SEQ ID NO:240, SEQ ID NO:241, and SEQ ID NO:242.

29-38. (canceled)
